SEQUENCE LISTING (439 sequences)

SEQ ID NO: 1 - protein, 115 aa, synthetic construct
  source 1..115: note = 20-H19-AB Heavy Chain
EVQLVESGGGLVQPKGSLKLSCAASGFTFNIYAMHWVRQAPGKGLEWIARIGSKSSYYATYYADSVKDRFTISRDDSQSMLYLQTNNLKTEDTAMYYCVSGKGDWGQGTTLTVSS

SEQ ID NO: 2 - protein, 112 aa, synthetic construct
  source 1..112: note = 20-H19-AB Light Chain
DVVMTQTPLTLSVIIGQPASISCKSSQSLLAGDGKTYLNWLLQRPGQSPKRLIYLVSKLDSGVPDRFTGSGSGTDFTLKISRVEAEDLGVYYCWQGTHFPWTFGAGTRLEIK

SEQ ID NO: 3 - protein, 115 aa, synthetic construct
  source 1..115: note = TP-43327F Heavy Chain
QVQLVESGGGVVQPGGSLRLSCAASGFTFNIYAMHWVRQAPGKGLEWVARIGSKSSYYATYYADSVKGRFTISRDNSKNTLYLQMNSLRTEDTAVYYCVSGKGDWGQGTLVTVSS

SEQ ID NO: 4 - protein, 112 aa, synthetic construct
  source 1..112: note = TP-43327F Light Chain
DVVMTQSPLSLPVTLGQPASISCRSSQSLLAGDGKTYLNWFLQRPGQSPRRLIYLVSKLDSGVPDRFSGSGSGTDFTLKISRVEAEDVGVYYCWQGTHFPWTFGAGTRLEIK

SEQ ID NO: 5 - protein, 115 aa, synthetic construct; identical to SEQ ID NO: 3
  source 1..115: note = TP-43329F Heavy Chain

SEQ ID NO: 6 - protein, 112 aa, synthetic construct
  source 1..112: note = TP-43329F Light Chain
DVVMTQSPLSLPVTLGQPASISCRSSQSLLAGDGKTYLNWFQQRPGQSPRRLIYLVSKLDSGVPDRFSGSGSGTDFTLKISRVEAEDVGVYYCWQGTHFPWTFGAGTRLEIK

SEQ ID NO: 7 - protein, 123 aa, synthetic construct
  source 1..123: note = 8-G17-A Heavy Chain
EVQLVESGGGLVKPGGSLKLSCEASGITFSNYVMSWVRQTPEKRLEWVATISDGGSYTYYPDNLKGRFTISRDNAKNNLYLQMSHLKSEDTAMYYCVRDRYDYDGRVYAMDYWGQGTSVTVSS

SEQ ID NO: 8 - protein, 112 aa, synthetic construct
  source 1..112: note = 8-G17-A Light Chain
DVVMTQTPLSLPVSLGDQASISCRSSQSLVHSNGNTYLHWYLQKPGQSPKLLIYKVSNRFSGVPDRFSGSGSGTDFTLKITRVEAEDLGVYFCSQSTHVPWTFGGGTKLEIK

SEQ ID NO: 9 - protein, 123 aa, synthetic construct
  source 1..123: note = 6-N03-A Heavy Chain
EVQLVESGGGLVKPGGSLKLSCEASGITFSNYVMSWVRQTPEKRLEWVATISDGGSYTYYPDNVKGRFTISRDNAKNNLYLQMSHLKSEDTAMYYCVRDRYDYDGRVYAMDYWGQGTSVTVSS

SEQ ID NO: 10 - protein, 112 aa, synthetic construct
  source 1..112: note = 6-N03-A Light Chain
DVVMTQTPLSLPVSLGDQASISCRSSQSLVHSNGDTYLHWYLQKPGQSPKLLIYKVSNRFSGVPDRFSGSGSGTDFTLKITRVEAEDLGVYFCSQSTHVPWTFGGGTKLEIK

SEQ ID NO: 11 - protein, 120 aa, synthetic construct
  source 1..120: note = R3-P3-C11 Heavy Chain
EVQLVESGGGLVQPGRSLRLACEASGFTFNNYAVHWVRQAPGKGLEWVAVISYDGTNKYYSDSMKGRFTISRDNSKNTLYLQINSLRVEDTAVYFCARGNYVRRDSFDIWGQGTMVTVSS

SEQ ID NO: 12 - protein, 107 aa, synthetic construct
  source 1..107: note = R3-P3-C11 Light Chain
DIQMTQSPPSLSASVGDRVTITCRASQSISSYLNWYQQKPGKAPKLLIYGASSLQSGVPLRFSGSGSGADFTLTISSLQPEDFATYYCQQSYISPITFGQGTRLEIK

SEQ ID NO: 13 - protein, 123 aa, synthetic construct
  source 1..123: note = R4M-P3-E06 Heavy Chain
EVQLVESGGGVVQPGGSLRLSCAASGFTFSNYGMHWVRQAPGKGLEWVATISYDGTKKYYPDSVRGRFTISRDNSENMLYLQMDSLRLEDTAVIYCAKDLVSGSRYGFAFDSWGPGTLVTVSS

SEQ ID NO: 14 - protein, 112 aa, synthetic construct
  source 1..112: note = R4M-P3-E06 Light Chain
DIVMTQSPLSLPVTLGQPASISCRSSQSLVHSDGNTYLNWFQQRPGQSPRRLIYKVSNRDSGVPDRFSGTGSGTDFTLKISRVEADDVGVYYCMQGTHWPPIFGQGTRLEIK

SEQ ID NO: 15 - protein, 123 aa, synthetic construct
  source 1..123: note = R3-P3-E09 Heavy Chain
EVQLVETGGGVVQPGRSLTLSCGASEFTFSNYAMHWVRQAPGKGLEWVAVISYDGTKKYYGDSVKGRFTISRENIKNTLYLQMNSLRSEDTAVYYCARDVGKGVTATGTFDIWGQGTMVTVSS

SEQ ID NO: 16 - protein, 107 aa, synthetic construct
  source 1..107: note = R3-P3-E09 Light Chain
AIRMTQSPSSLSASVGDRVTITCRASQGIGSYLAWFQQKPGEAPKSLIIDATRLKSGVPSRFSGSGSGTEFALTISSLRPEDFATYYCQQYNVSPLTFGGGTKVEIK

SEQ ID NO: 17 - protein, 113 aa, synthetic construct
  source 1..113: note = R3-P1-C02 Heavy Chain
EVQLVESGGGVVQPGGSLRISCVASEFPFSDFGMHWVRQAPGKGLEWVAFVRSHGSGKYYADSVKGRFTISGDKSKSTLYLQMESLRPDDAAVYYCTTLADVWGQGTTVTVSS

SEQ ID NO: 18 - protein, 112 aa, synthetic construct
  source 1..112: note = R3-P1-C02 Light Chain
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPWTFGQGTKVEIK

SEQ ID NO: 19 - protein, 122 aa, synthetic construct
  source 1..122: note = R3-P3-A12 Heavy Chain
EVQLVESGGGLVQPGRSLRLSCGVSGFTFSSFGMHWVRQAPGKGLEWVAVISNDALHKNYGDSVKGRFTVTRDNSRNTLFLQMTSLRPEDTAVYYCAKDVVRGHTSGSLGSWGQGTLVTVSS

SEQ ID NO: 20 - protein, 112 aa, synthetic construct
  source 1..112: note = R3-P3-A12 Light Chain
DVVMTQSPLSLPVTPGEPASISCRSSQSLLYSNGYNYLDWYLQKPGQSPQLLIYLGSNRASGVPDRFSGSGSGTDFTLKISRVEAEDVGVYYCMQALQTPLTFGGGTKLEIK

SEQ ID NO: 21 - protein, 120 aa, synthetic construct
  source 1..120: note = R4M-P3-A12 Heavy Chain
EVQLVESGGGVVQPGGSLRLSCAASGFTFSDHYMSWIRQAPGKGLEWVSYLSGSGTSIYYADSVKGRFTISRDNAKKSLYLQMNSLRAEDTAVYYCARVGPAAGNAFDIWGQGTMVTVSS

SEQ ID NO: 22 - protein, 110 aa, synthetic construct
  source 1..110: note = R4M-P3-A12 Light Chain
QSVLTQPPSASGTPGQRVTISCSGSSSNIGSNYVYWYQQLPGTAPKLLIYRNNQRPSGVPDRFSGSKSGTSASLAITGLQAEDEADYYCQSYDSSLSGVVFGGGTKLTVL

SEQ ID NO: 23 - protein, 119 aa, synthetic construct
  source 1..119: note = R4M-P1-A10 Heavy Chain
EVQLVETGGGVVQPGRSLRLSCAASGFTFSSYAMHWVRQAPGKGLEWVAVISYDGSNKYYADSVKGRFTISRDNSKNTLYLQMNSLRAEDTAVYYCARDRGWADAFDIWGQGTMVTVSS

SEQ ID NO: 24 - protein, 110 aa, synthetic construct
  source 1..110: note = R4M-P1-A10 Light Chain
QAGLTQPPSVSKGLRQTATLTCTGNSNNVGYQGAVWLLQYQGHPPKVLSNRNNDRPSGISERLSASRSGNTASLTITGLQPEDEGDYFCAAWDGSLRGWVFGGGTKLTVL

SEQ ID NO: 25 - protein, 201 aa, synthetic construct
  source 1..201: note = Human Sdc2 protein
MRRAWILLTLGLVACVSAESRAELTSDKDMYLDNSSIEEASGVYPIDDDDYASASGSGADEDVESPELTTSRPLPKILLTSAAPKVETTTLNIQNKIPAQTKSPEETDKEKVHLSDSERKMDPAEEDTNVYTEKHSDSLFKRTEVLAAVIAGGVIGFLFAIFLILLLVYRMRKKDEGSYDLGERKPSSAAYQKAPTKEFYA

SEQ ID NO: 26 - protein, 202 aa, synthetic construct
  source 1..202: note = Mouse Sdc2 protein
MQRAWILLTLGLMACVSAETRTELTSDKDMYLDNSSIEEASGVYPIDDDDYSSASGSGADEDIESPVLTTSQLIPRIPLTSAASPKVETMTLKTQSITPAQTESPEETDKEEVDISEAEEKLGPAIKSTDVYTEKHSDNLFKRTEVLAAVIAGGVIGFLFAIFLILLLVYRMRKKDEGSYDLGERKPSSAAYQKAPTKEFYA

SEQ ID NO: 27 - protein, 137 aa, synthetic construct
  source 1..137: note = Extracellular domain of human syndecan-2
MYLDNSSIEEASGVYPIDDDDYASASGSGADEDVESPELTTSRPLPKILLTSAAPKVETTTLNIQNKIPAQTKSPEETDKEKVHLSDSERKMDPAEEDTNVYTEKHSDSLFKRTEVLAAVIAGGVIGFLFAIFLILL

SEQ ID NO: 28 - protein, 18 aa, synthetic construct
  source 1..18: note = huSdc2 peptide
PAEEDTNVYTEKHSDSLF

SEQ ID NO: 29 - protein, 18 aa, synthetic construct
  source 1..18: note = mSdc2 peptide
PAIKSTDVYTEKHSDNLF

SEQ ID NO: 30 - protein, 18 aa, synthetic construct
  source 1..18: note = pSdc2 peptide
PAEKDTNVYTEKHSDNLF

SEQ ID NO: 31 - protein, 5 aa, synthetic construct
  source 1..5: note = 20-H19-AB HC Kabat CDR1
IYAMH

SEQ ID NO: 32 - protein, 19 aa, synthetic construct
  source 1..19: note = 20-H19-AB HC Kabat CDR2
RIGSKSSYYATYYADSVKD

SEQ ID NO: 33 - protein, 4 aa, synthetic construct
  source 1..4: note = 20-H19-AB HC Kabat CDR3
GKGD

SEQ ID NO: 34 - protein, 16 aa, synthetic construct
  source 1..16: note = 20-H19-AB LC Kabat CDR1
KSSQSLLAGDGKTYLN

SEQ ID NO: 35 - protein, 7 aa, synthetic construct
  source 1..7: note = 20-H19-AB LC Kabat CDR2
LVSKLDS

SEQ ID NO: 36 - protein, 9 aa, synthetic construct
  source 1..9: note = 20-H19-AB LC Kabat CDR3
WQGTHFPWT

SEQ ID NO: 37 - protein, 7 aa, synthetic construct
  source 1..7: note = 20-H19-AB HC Chothia CDR1
GFTFNIY

SEQ ID NO: 38 - protein, 8 aa, synthetic construct
  source 1..8: note = 20-H19-AB HC Chothia CDR2
GSKSSYYA

SEQ ID NO: 39 - protein, 4 aa, synthetic construct
  source 1..4: note = 20-H19-AB HC Chothia CDR3
GKGD

SEQ ID NO: 40 - protein, 16 aa, synthetic construct
  source 1..16: note = 20-H19-AB LC Chothia CDR1
KSSQSLLAGDGKTYLN

SEQ ID NO: 41 - protein, 7 aa, synthetic construct
  source 1..7: note = 20-H19-AB LC Chothia CDR2
LVSKLDS

SEQ ID NO: 42 - protein, 9 aa, synthetic construct
  source 1..9: note = 20-H19-AB LC Chothia CDR3
WQGTHFPWT

SEQ ID NO: 43 - protein, 10 aa, synthetic construct
  source 1..10: note = 20-H19-AB HC AbM CDR1
GFTFNIYAMH

SEQ ID NO: 44 - protein, 12 aa, synthetic construct
  source 1..12: note = 20-H19-AB HC AbM CDR2
RIGSKSSYYATY

SEQ ID NO: 45 - protein, 4 aa, synthetic construct
  source 1..4: note = 20-H19-AB HC AbM CDR3
GKGD

SEQ ID NO: 46 - protein, 16 aa, synthetic construct
  source 1..16: note = 20-H19-AB LC AbM CDR1
KSSQSLLAGDGKTYLN

SEQ ID NO: 47 - protein, 7 aa, synthetic construct
  source 1..7: note = 20-H19-AB LC AbM CDR2
LVSKLDS

SEQ ID NO: 48 - protein, 9 aa, synthetic construct
  source 1..9: note = 20-H19-AB LC AbM CDR3
WQGTHFPWT

SEQ ID NO: 49 - protein, 6 aa, synthetic construct
  source 1..6: note = 20-H19-AB HC Contact CDR1
NIYAMH

SEQ ID NO: 50 - protein, 15 aa, synthetic construct
  source 1..15: note = 20-H19-AB HC Contact CDR2
WIARIGSKSSYYATY

SEQ ID NO: 51 - protein, 5 aa, synthetic construct
  source 1..5: note = 20-H19-AB HC Contact CDR3
VSGKG

SEQ ID NO: 52 - protein, 12 aa, synthetic construct
  source 1..12: note = 20-H19-AB LC Contact CDR1
LAGDGKTYLNWL

SEQ ID NO: 53 - protein, 10 aa, synthetic construct
  source 1..10: note = 20-H19-AB LC Contact CDR2
RLIYLVSKLD

SEQ ID NO: 54 - protein, 8 aa, synthetic construct
  source 1..8: note = 20-H19-AB LC Contact CDR3
WQGTHFPW

SEQ ID NO: 55 - protein, 8 aa, synthetic construct
  source 1..8: note = 20-H19-AB HC IMGT CDR1
GFTFNIYA

SEQ ID NO: 56 - protein, 10 aa, synthetic construct
  source 1..10: note = 20-H19-AB HC IMGT CDR2
IGSKSSYYAT

SEQ ID NO: 57 - protein, 6 aa, synthetic construct
  source 1..6: note = 20-H19-AB HC IMGT CDR3
VSGKGD

SEQ ID NO: 58 - protein, 11 aa, synthetic construct
  source 1..11: note = 20-H19-AB LC IMGT CDR1
QSLLAGDGKTY

SEQ ID NO: 59 - skipped

SEQ ID NO: 60 - protein, 9 aa, synthetic construct
  source 1..9: note = 20-H19-AB LC IMGT CDR3
WQGTHFPWT

SEQ ID NO: 61 - protein, 115 aa, synthetic construct; identical to SEQ ID NO: 1
  source 1..115: note = 20-H19-AB IgG1 Kappa VH AA

SEQ ID NO: 62 - protein, 112 aa, synthetic construct; identical to SEQ ID NO: 2
  source 1..112: note = 20-H19-AB IgG1 Kappa VL AA

SEQ ID NO: 63 - protein, 462 aa, synthetic construct
  source 1..462: note = 20-H19-AB IgG1 Kappa Heavy Chain AA
MDPKGSLSWRILLFLSLAFELSYGEVQLVESGGGLVQPKGSLKLSCAASGFTFNIYAMHWVRQAPGKGLEWIARIGSKSSYYATYYADSVKDRFTISRDDSQSMLYLQTNNLKTEDTAMYYCVSGKGDWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 64 - protein, 239 aa, synthetic construct
  source 1..239: note = 20-H19-AB IgG1 Kappa Light Chain AA
METDTLLLWVLLLWVPGSTGDVVMTQTPLTLSVIIGQPASISCKSSQSLLAGDGKTYLNWLLQRPGQSPKRLIYLVSKLDSGVPDRFTGSGSGTDFTLKISRVEAEDLGVYYCWQGTHFPWTFGAGTRLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 65 - protein, 5 aa, synthetic construct
  source 1..5: note = TP-43327F HC Kabat CDR1
IYAMH

SEQ ID NO: 66 - protein, 19 aa, synthetic construct
  source 1..19: note = TP-43327F HC Kabat CDR2
RIGSKSSYYATYYADSVKG

SEQ ID NO: 67 - protein, 4 aa, synthetic construct
  source 1..4: note = TP-43327F HC Kabat CDR3
GKGD

SEQ ID NO: 68 - protein, 16 aa, synthetic construct
  source 1..16: note = TP-43327F LC Kabat CDR1
RSSQSLLAGDGKTYLN

SEQ ID NO: 69 - protein, 7 aa, synthetic construct
  source 1..7: note = TP-43327F LC Kabat CDR2
LVSKLDS

SEQ ID NO: 70 - protein, 9 aa, synthetic construct
  source 1..9: note = TP-43327F LC Kabat CDR3
WQGTHFPWT

SEQ ID NO: 71 - protein, 7 aa, synthetic construct
  source 1..7: note = TP-43327F HC Chothia CDR1
GFTFNIY

SEQ ID NO: 72 - protein, 8 aa, synthetic construct
  source 1..8: note = TP-43327F HC Chothia CDR2
GSKSSYYA

SEQ ID NO: 73 - protein, 4 aa, synthetic construct
  source 1..4: note = TP-43327F HC Chothia CDR3
GKGD

SEQ ID NO: 74 - protein, 16 aa, synthetic construct
  source 1..16: note = TP-43327F LC Chothia CDR1
RSSQSLLAGDGKTYLN

SEQ ID NO: 75 - protein, 7 aa, synthetic construct
  source 1..7: note = TP-43327F LC Chothia CDR2
LVSKLDS

SEQ ID NO: 76 - protein, 9 aa, synthetic construct
  source 1..9: note = TP-43327F LC Chothia CDR3
WQGTHFPWT

SEQ ID NO: 77 - protein, 10 aa, synthetic construct
  source 1..10: note = TP-43327F HC AbM CDR1
GFTFNIYAMH

SEQ ID NO: 78 - protein, 12 aa, synthetic construct
  source 1..12: note = TP-43327F HC AbM CDR2
RIGSKSSYYATY

SEQ ID NO: 79 - protein, 4 aa, synthetic construct
  source 1..4: note = TP-43327F HC AbM CDR3
GKGD

SEQ ID NO: 80 - protein, 16 aa, synthetic construct
  source 1..16: note = TP-43327F LC AbM CDR1
RSSQSLLAGDGKTYLN

SEQ ID NO: 81 - protein, 7 aa, synthetic construct
  source 1..7: note = TP-43327F LC AbM CDR2
LVSKLDS

SEQ ID NO: 82 - protein, 9 aa, synthetic construct
  source 1..9: note = TP-43327F LC AbM CDR3
WQGTHFPWT

SEQ ID NO: 83 - protein, 6 aa, synthetic construct
  source 1..6: note = TP-43327F HC Contact CDR1
NIYAMH

SEQ ID NO: 84 - protein, 15 aa, synthetic construct
  source 1..15: note = TP-43327F HC Contact CDR2
WVARIGSKSSYYATY

SEQ ID NO: 85 - protein, 5 aa, synthetic construct
  source 1..5: note = TP-43327F HC Contact CDR3
VSGKG

SEQ ID NO: 86 - protein, 12 aa, synthetic construct
  source 1..12: note = TP-43327F LC Contact CDR1
LAGDGKTYLNWF

SEQ ID NO: 87 - protein, 10 aa, synthetic construct
  source 1..10: note = TP-43327F LC Contact CDR2
RLIYLVSKLD

SEQ ID NO: 88 - protein, 8 aa, synthetic construct
  source 1..8: note = TP-43327F LC Contact CDR3
WQGTHFPW

SEQ ID NO: 89 - protein, 8 aa, synthetic construct
  source 1..8: note = TP-43327F HC IMGT CDR1
GFTFNIYA

SEQ ID NO: 90 - protein, 10 aa, synthetic construct
  source 1..10: note = TP-43327F HC IMGT CDR2
IGSKSSYYAT

SEQ ID NO: 91 - protein, 6 aa, synthetic construct
  source 1..6: note = TP-43327F HC IMGT CDR3
VSGKGD

SEQ ID NO: 92 - protein, 11 aa, synthetic construct
  source 1..11: note = TP-43327F LC IMGT CDR1
QSLLAGDGKTY

SEQ ID NO: 93 - skipped

SEQ ID NO: 94 - protein, 9 aa, synthetic construct
  source 1..9: note = TP-43327F LC IMGT CDR3
WQGTHFPWT

SEQ ID NO: 95 - protein, 115 aa, synthetic construct; identical to SEQ ID NO: 3
  source 1..115: note = TP-43327F IgG1 Kappa VH AA

SEQ ID NO: 96 - protein, 112 aa, synthetic construct; identical to SEQ ID NO: 4
  source 1..112: note = TP-43327F IgG1 Kappa VL AA

SEQ ID NO: 97 - protein, 468 aa, synthetic construct
  source 1..468: note = TP-43327F IgG1 Kappa Heavy Chain AA
MDPKGSLSWRILLFLSLAFELSYGQVQLVESGGGVVQPGGSLRLSCAASGFTFNIYAMHWVRQAPGKGLEWVARIGSKSSYYATYYADSVKGRFTISRDNSKNTLYLQMNSLRTEDTAVYYCVSGKGDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 98 - protein, 239 aa, synthetic construct
  source 1..239: note = TP-43327F IgG1 Kappa Light Chain AA
METDTLLLWVLLLWVPGSTGDVVMTQSPLSLPVTLGQPASISCRSSQSLLAGDGKTYLNWFLQRPGQSPRRLIYLVSKLDSGVPDRFSGSGSGTDFTLKISRVEAEDVGVYYCWQGTHFPWTFGAGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 5 aa, synthetic construct
  source 1..5: note = TP-43329F HC Kabat CDR1
IYAMH

SEQ ID NO: 100 - protein, 19 aa, synthetic construct
  source 1..19: note = TP-43329F HC Kabat CDR2
RIGSKSSYYATYYADSVKG

SEQ ID NO: 101 - protein, 4 aa, synthetic construct
  source 1..4: note = TP-43329F HC Kabat CDR3
GKGD

SEQ ID NO: 102 - protein, 16 aa, synthetic construct
  source 1..16: note = TP-43329F LC Kabat CDR1
RSSQSLLAGDGKTYLN

SEQ ID NO: 103 - protein, 16 aa, synthetic construct
  source 1..16: note = TP-43329F LC Kabat CDR2
RSSQSLLAGDGKTYLN

SEQ ID NO: 104 - protein, 9 aa, synthetic construct
  source 1..9: note = TP-43329F LC Kabat CDR3
WQGTHFPWT

SEQ ID NO: 105 - protein, 7 aa, synthetic construct
  source 1..7: note = TP-43329F HC Chothia CDR1
GFTFNIY

SEQ ID NO: 106 - protein, 8 aa, synthetic construct
  source 1..8: note = TP-43329F HC Chothia CDR2
GSKSSYYA

SEQ ID NO: 107 - protein, 4 aa, synthetic construct
  source 1..4: note = TP-43329F HC Chothia CDR3
GKGD

SEQ ID NO: 108 - protein, 16 aa, synthetic construct
  source 1..16: note = TP-43329F LC Chothia CDR1
RSSQSLLAGDGKTYLN

SEQ ID NO: 109 - protein, 7 aa, synthetic construct
  source 1..7: note = TP-43329F LC Chothia CDR2
LVSKLDS

SEQ ID NO: 110 - protein, 9 aa, synthetic construct
  source 1..9: note = TP-43329F LC Chothia CDR3
WQGTHFPWT

SEQ ID NO: 111 - protein, 10 aa, synthetic construct
  source 1..10: note = TP-43329F HC AbM CDR1
GFTFNIYAMH

SEQ ID NO: 112 - protein, 12 aa, synthetic construct
  source 1..12: note = TP-43329F HC AbM CDR2
RIGSKSSYYATY

SEQ ID NO: 113 - protein, 4 aa, synthetic construct
  source 1..4: note = TP-43329F HC AbM CDR3
GKGD

SEQ ID NO: 114 - protein, 16 aa, synthetic construct
  source 1..16: note = TP-43329F LC AbM CDR1
RSSQSLLAGDGKTYLN

SEQ ID NO: 115 - protein, 7 aa, synthetic construct
  source 1..7: note = TP-43329F LC AbM CDR2
LVSKLDS

SEQ ID NO: 116 - protein, 9 aa, synthetic construct
  source 1..9: note = TP-43329F LC AbM CDR3
WQGTHFPWT

SEQ ID NO: 117 - protein, 6 aa, synthetic construct
  source 1..6: note = TP-43329F HC Contact CDR1
NIYAMH

SEQ ID NO: 118 - protein, 15 aa, synthetic construct
  source 1..15: note = TP-43329F HC Contact CDR2
WVARIGSKSSYYATY

SEQ ID NO: 119 - protein, 5 aa, synthetic construct
  source 1..5: note = TP-43329F HC Contact CDR3
VSGKG

SEQ ID NO: 120 - protein, 12 aa, synthetic construct
  source 1..12: note = TP-43329F LC Contact CDR1
LAGDGKTYLNWF

SEQ ID NO: 121 - protein, 10 aa, synthetic construct
  source 1..10: note = TP-43329F LC Contact CDR2
RLIYLVSKLD

SEQ ID NO: 122 - protein, 8 aa, synthetic construct
  source 1..8: note = TP-43329F LC Contact CDR3
WQGTHFPW

SEQ ID NO: 123 - protein, 8 aa, synthetic construct
  source 1..8: note = TP-43329F HC IMGT CDR1
GFTFNIYA

SEQ ID NO: 124 - protein, 10 aa, synthetic construct
  source 1..10: note = TP-43329F HC IMGT CDR2
IGSKSSYYAT

SEQ ID NO: 125 - protein, 6 aa, synthetic construct
  source 1..6: note = TP-43329F HC IMGT CDR3
VSGKGD

SEQ ID NO: 126 - protein, 11 aa, synthetic construct
  source 1..11: note = TP-43329F LC IMGT CDR1
QSLLAGDGKTY

SEQ ID NO: 127 - skipped

SEQ ID NO: 128 - protein, 9 aa, synthetic construct
  source 1..9: note = TP-43329F LC IMGT CDR3
WQGTHFPWT

SEQ ID NO: 129 - protein, 115 aa, synthetic construct; identical to SEQ ID NO: 3
  source 1..115: note = TP-43329F IgG1 Kappa VH AA

SEQ ID NO: 130 - protein, 112 aa, synthetic construct; identical to SEQ ID NO: 6
  source 1..112: note = TP-43329F IgG1 Kappa VL AA

SEQ ID NO: 131 - protein, 468 aa, synthetic construct; identical to SEQ ID NO: 97
  source 1..468: note = TP-43329F IgG1 Kappa Heavy Chain AA

SEQ ID NO: 132 - protein, 239 aa, synthetic construct
  source 1..239: note = TP-43329F IgG1 Kappa Light Chain AA
METDTLLLWVLLLWVPGSTGDVVMTQSPLSLPVTLGQPASISCRSSQSLLAGDGKTYLNWFQQRPGQSPRRLIYLVSKLDSGVPDRFSGSGSGTDFTLKISRVEAEDVGVYYCWQGTHFPWTFGAGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 5 aa, synthetic construct
  source 1..5: note = 8-G17-A HC Kabat CDR1
NYVMS

SEQ ID NO: 134 - protein, 17 aa, synthetic construct
  source 1..17: note = 8-G17-A HC Kabat CDR2
TISDGGSYTYYPDNLKG

SEQ ID NO: 135 - protein, 14 aa, synthetic construct
  source 1..14: note = 8-G17-A HC Kabat CDR3
DRYDYDGRVYAMDY

SEQ ID NO: 136 - protein, 16 aa, synthetic construct
  source 1..16: note = 8-G17-A LC Kabat CDR1
RSSQSLVHSNGNTYLH

SEQ ID NO: 137 - protein, 7 aa, synthetic construct
  source 1..7: note = 8-G17-A LC Kabat CDR2
KVSNRFS

SEQ ID NO: 138 - protein, 9 aa, synthetic construct
  source 1..9: note = 8-G17-A LC Kabat CDR3
SQSTHVPWT

SEQ ID NO: 139 - protein, 7 aa, synthetic construct
  source 1..7: note = 8-G17-A HC Chothia CDR1
GITFSNY

SEQ ID NO: 140 - protein, 6 aa, synthetic construct
  source 1..6: note = 8-G17-A HC Chothia CDR2
SDGGSY

SEQ ID NO: 141 - protein, 14 aa, synthetic construct
  source 1..14: note = 8-G17-A HC Chothia CDR3
DRYDYDGRVYAMDY

SEQ ID NO: 142 - protein, 16 aa, synthetic construct
  source 1..16: note = 8-G17-A LC Chothia CDR1
RSSQSLVHSNGNTYLH

SEQ ID NO: 143 - protein, 7 aa, synthetic construct
  source 1..7: note = 8-G17-A LC Chothia CDR2
KVSNRFS

SEQ ID NO: 144 - protein, 9 aa, synthetic construct
  source 1..9: note = 8-G17-A LC Chothia CDR3
SQSTHVPWT

SEQ ID NO: 145 - protein, 10 aa, synthetic construct
  source 1..10: note = 8-G17-A HC AbM CDR1
GITFSNYVMS

SEQ ID NO: 146 - protein, 10 aa, synthetic construct
  source 1..10: note = 8-G17-A HC AbM CDR2
TISDGGSYTY

SEQ ID NO: 147 - protein, 14 aa, synthetic construct
  source 1..14: note = 8-G17-A HC AbM CDR3
DRYDYDGRVYAMDY

SEQ ID NO: 148 - protein, 16 aa, synthetic construct
  source 1..16: note = 8-G17-A LC AbM CDR1
RSSQSLVHSNGNTYLH

SEQ ID NO: 149 - protein, 7 aa, synthetic construct
  source 1..7: note = 8-G17-A LC AbM CDR2
KVSNRFS

SEQ ID NO: 150 - protein, 9 aa, synthetic construct
  source 1..9: note = 8-G17-A LC AbM CDR3
SQSTHVPWT

SEQ ID NO: 151 - protein, 6 aa, synthetic construct
  source 1..6: note = 8-G17-A HC Contact CDR1
SNYVMS

SEQ ID NO: 152 - protein, 13 aa, synthetic construct
  source 1..13: note = 8-G17-A HC Contact CDR2
WVATISDGGSYTY

SEQ ID NO: 153 - protein, 15 aa, synthetic construct
  source 1..15: note = 8-G17-A HC Contact CDR3
VRDRYDYDGRVYAMD

SEQ ID NO: 154 - protein, 12 aa, synthetic construct
  source 1..12: note = 8-G17-A LC Contact CDR1
VHSNGNTYLHWY

SEQ ID NO: 155 - protein, 10 aa, synthetic construct
  source 1..10: note = 8-G17-A LC Contact CDR2
LLIYKVSNRF

SEQ ID NO: 156 - protein, 8 aa, synthetic construct
  source 1..8: note = 8-G17-A LC Contact CDR3
SQSTHVPW

SEQ ID NO: 157 - protein, 8 aa, synthetic construct
  source 1..8: note = 8-G17-A HC IMGT CDR1
GITFSNYV

SEQ ID NO: 158 - protein, 8 aa, synthetic construct
  source 1..8: note = 8-G17-A HC IMGT CDR2
ISDGGSYT

SEQ ID NO: 159 - protein, 16 aa, synthetic construct
  source 1..16: note = 8-G17-A HC IMGT CDR3
VRDRYDYDGRVYAMDY

SEQ ID NO: 160 - protein, 11 aa, synthetic construct
  source 1..11: note = 8-G17-A LC IMGT CDR1
QSLVHSNGNTY

SEQ ID NO: 161 - skipped

SEQ ID NO: 162 - protein, 9 aa, synthetic construct
  source 1..9: note = 8-G17-A LC IMGT CDR3
SQSTHVPWT

SEQ ID NO: 163 - protein, 123 aa, synthetic construct; identical to SEQ ID NO: 7
  source 1..123: note = 8-G17-A IgG3 Kappa VH AA

SEQ ID NO: 164 - protein, 112 aa, synthetic construct; identical to SEQ ID NO: 8
  source 1..112: note = 8-G17-A IgG3 Kappa VL AA

SEQ ID NO: 165 - protein, 476 aa, synthetic construct
  source 1..476: note = 8-G17-A IgG3 Kappa Heavy Chain AA
MDPKGSLSWRILLFLSLAFELSYGEVQLVESGGGLVKPGGSLKLSCEASGITFSNYVMSWVRQTPEKRLEWVATISDGGSYTYYPDNLKGRFTISRDNAKNNLYLQMSHLKSEDTAMYYCVRDRYDYDGRVYAMDYWGQGTSVTVSSATTTAPSVYPLVPGCSDTSGSSVTLGCLVKGYFPEPVTVKWNYGALSSGVRTVSSVLQSGFYSLSSLVTVPSSTWPSQTVICNVAHPASKTELIKRIEPRIPKPSTPPGSSCPPGNILGGPSVFIFPPKPKDALMISLTPKVTCVVVDVSEDDPDVHVSWFVDNKEVHTAWTQPREAQYNSTFRVVSALPIQHQDWMRGKEFKCKVNNKALPAPIERTISKPKGRAQTPQVYTIPPPREQMSKKKVSLTCLVTNFFSEAISVEWERNGELEQDYKNTPPILDSDGTYFLYSKLTVDTDSWLQGEIFTCSVVHEALHNHHTQKNLSRSPG

SEQ ID NO: 166 - protein, 239 aa, synthetic construct
  source 1..239: note = 8-G17-A IgG3 Kappa Light Chain AA
METDTLLLWVLLLWVPGSTGDVVMTQTPLSLPVSLGDQASISCRSSQSLVHSNGNTYLHWYLQKPGQSPKLLIYKVSNRFSGVPDRFSGSGSGTDFTLKITRVEAEDLGVYFCSQSTHVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 167 - protein, 5 aa, synthetic construct
  source 1..5: note = 6-N03-A HC Kabat CDR1
NYVMS

SEQ ID NO: 168 - protein, 17 aa, synthetic construct
  source 1..17: note = 6-N03-A HC Kabat CDR2
TISDGGSYTYYPDNVKG

SEQ ID NO: 169 - protein, 14 aa, synthetic construct
  source 1..14: note = 6-N03-A HC Kabat CDR3
DRYDYDGRVYAMDY

SEQ ID NO: 170 - protein, 16 aa, synthetic construct
  source 1..16: note = 6-N03-A LC Kabat CDR1
RSSQSLVHSNGDTYLH

SEQ ID NO: 171 - protein, 7 aa, synthetic construct
  source 1..7: note = 6-N03-A LC Kabat CDR2
KVSNRFS

SEQ ID NO: 172 - protein, 9 aa, synthetic construct
  source 1..9: note = 6-N03-A LC Kabat CDR3
SQSTHVPWT

SEQ ID NO: 173 - protein, 7 aa, synthetic construct
  source 1..7: note = 6-N03-A HC Chothia CDR1
GITFSNY

SEQ ID NO: 174 - protein, 6 aa, synthetic construct
  source 1..6: note = 6-N03-A HC Chothia CDR2
SDGGSY

SEQ ID NO: 175 - protein, 14 aa, synthetic construct
  source 1..14: note = 6-N03-A HC Chothia CDR3
DRYDYDGRVYAMDY

SEQ ID NO: 176 - protein, 16 aa, synthetic construct
  source 1..16: note = 6-N03-A LC Chothia CDR1
RSSQSLVHSNGDTYLH

SEQ ID NO: 177 - protein, 7 aa, synthetic construct
  source 1..7: note = 6-N03-A LC Chothia CDR2
KVSNRFS

SEQ ID NO: 178 - protein, 9 aa, synthetic construct
  source 1..9: note = 6-N03-A LC Chothia CDR3
SQSTHVPWT

SEQ ID NO: 179 - protein, 10 aa, synthetic construct
  source 1..10: note = 6-N03-A HC AbM CDR1
GITFSNYVMS

SEQ ID NO: 180 - protein, 10 aa, synthetic construct
  source 1..10: note = 6-N03-A HC AbM CDR2
TISDGGSYTY

SEQ ID NO: 181 - protein, 14 aa, synthetic construct
  source 1..14: note = 6-N03-A HC AbM CDR3
DRYDYDGRVYAMDY

SEQ ID NO: 182 - protein, 16 aa, synthetic construct
  source 1..16: note = 6-N03-A LC AbM CDR1
RSSQSLVHSNGDTYLH

SEQ ID NO: 183 - protein, 7 aa, synthetic construct
  source 1..7: note = 6-N03-A LC AbM CDR2
KVSNRFS

SEQ ID NO: 184 - protein, 9 aa, synthetic construct
  source 1..9: note = 6-N03-A LC AbM CDR3
SQSTHVPWT

SEQ ID NO: 185 - protein, 6 aa, synthetic construct
  source 1..6: note = 6-N03-A HC Contact CDR1
SNYVMS

SEQ ID NO: 186 - protein, 13 aa, synthetic construct
  source 1..13: note = 6-N03-A HC Contact CDR2
WVATISDGGSYTY

SEQ ID NO: 187 - protein, 15 aa, synthetic construct
  source 1..15: note = 6-N03-A HC Contact CDR3
VRDRYDYDGRVYAMD

SEQ ID NO: 188 - protein, 12 aa, synthetic construct
  source 1..12: note = 6-N03-A LC Contact CDR1
VHSNGDTYLHWY

SEQ ID NO: 189 - protein, 10 aa, synthetic construct
  source 1..10: note = 6-N03-A LC Contact CDR2
LLIYKVSNRF

SEQ ID NO: 190 - protein, 8 aa, synthetic construct
  source 1..8: note = 6-N03-A LC Contact CDR3
SQSTHVPW

SEQ ID NO: 191 - protein, 8 aa, synthetic construct
  source 1..8: note = 6-N03-A HC IMGT CDR1
GITFSNYV

SEQ ID NO: 192 - protein, 8 aa, synthetic construct
  source 1..8: note = 6-N03-A HC IMGT CDR2
ISDGGSYT

SEQ ID NO: 193 - protein, 16 aa, synthetic construct
  source 1..16: note = 6-N03-A HC IMGT CDR3
VRDRYDYDGRVYAMDY

SEQ ID NO: 194 - protein, 11 aa, synthetic construct
  source 1..11: note = 6-N03-A LC IMGT CDR1
QSLVHSNGDTY

SEQ ID NO: 195 - skipped

SEQ ID NO: 196 - protein, 9 aa, synthetic construct
  source 1..9: note = 6-N03-A LC IMGT CDR3
SQSTHVPWT

SEQ ID NO: 197 - protein, 123 aa, synthetic construct; identical to SEQ ID NO: 9
  source 1..123: note = 6-N03-A IgG1 Kappa VH AA

SEQ ID NO: 198 - protein, 112 aa, synthetic construct; identical to SEQ ID NO: 10
  source 1..112: note = 6-N03-A IgG1 Kappa VL AA

SEQ ID NO: 199 - protein, 470 aa, synthetic construct
  source 1..470: note = 6-N03-A IgG1 Kappa Heavy Chain AA
MDPKGSLSWRILLFLSLAFELSYGEVQLVESGGGLVKPGGSLKLSCEASGITFSNYVMSWVRQTPEKRLEWVATISDGGSYTYYPDNVKGRFTISRDNAKNNLYLQMSHLKSEDTAMYYCVRDRYDYDGRVYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 200 - protein, 239 aa, synthetic construct
  source 1..239: note = 6-N03-A IgG1 Kappa Light Chain AA
METDTLLLWVLLLWVPGSTGDVVMTQTPLSLPVSLGDQASISCRSSQSLVHSNGDTYLHWYLQKPGQSPKLLIYKVSNRFSGVPDRFSGSGSGTDFTLKITRVEAEDLGVYFCSQSTHVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 201 - protein, 5 aa, synthetic construct
  source 1..5: note = R3-P3-C11 HC Kabat CDR1
NYAVH

SEQ ID NO: 202 - protein, 17 aa, synthetic construct
  source 1..17: note = R3-P3-C11 HC Kabat CDR2
VISYDGTNKYYSDSMKG

SEQ ID NO: 203 - protein, 11 aa, synthetic construct
  source 1..11: note = R3-P3-C11 HC Kabat CDR3
GNYVRRDSFDI

SEQ ID NO: 204 - protein, 11 aa, synthetic construct
  source 1..11: note = R3-P3-C11 LC Kabat CDR1
RASQSISSYLN

SEQ ID NO: 205 - protein, 7 aa, synthetic construct
  source 1..7: note = R3-P3-C11 LC Kabat CDR2
GASSLQS

SEQ ID NO: 206 - protein, 9 aa, synthetic construct
  source 1..9: note = R3-P3-C11 LC Kabat CDR3
QQSYISPIT

SEQ ID NO: 207 - protein, 7 aa, synthetic construct
  source 1..7: note = R3-P3-C11 HC Chothia CDR1
GFTFNNY

SEQ ID NO: 208 - protein, 6 aa, synthetic construct
  source 1..6: note = R3-P3-C11 HC Chothia CDR2
SYDGTN

SEQ ID NO: 209 - protein, 11 aa, synthetic construct
  source 1..11: note = R3-P3-C11 HC Chothia CDR3
GNYVRRDSFDI

SEQ ID NO: 210 - protein, 11 aa, synthetic construct
  source 1..11: note = R3-P3-C11 LC Chothia CDR1
RASQSISSYLN

SEQ ID NO: 211 - protein, 7 aa, synthetic construct
  source 1..7: note = R3-P3-C11 LC Chothia CDR2
GASSLQS

SEQ ID NO: 212 - protein, 9 aa, synthetic construct
  source 1..9: note = R3-P3-C11 LC Chothia CDR3
QQSYISPIT

SEQ ID NO: 213 - protein, 10 aa, synthetic construct
  source 1..10: note = R3-P3-C11 HC AbM CDR1
GFTFNNYAVH

SEQ ID NO: 214 - protein, 10 aa, synthetic construct
  source 1..10: note = R3-P3-C11 HC AbM CDR2
VISYDGTNKY

SEQ ID NO: 215 - protein, 11 aa, synthetic construct
  source 1..11: note = R3-P3-C11 HC AbM CDR3
GNYVRRDSFDI

SEQ ID NO: 216 - protein, 11 aa, synthetic construct
  source 1..11: note = R3-P3-C11 LC AbM CDR1
RASQSISSYLN

SEQ ID NO: 217 - protein, 7 aa, synthetic construct
  source 1..7: note = R3-P3-C11 LC AbM CDR2
GASSLQS

SEQ ID NO: 218 - protein, 9 aa, synthetic construct
  source 1..9: note = R3-P3-C11 LC AbM CDR3
QQSYISPIT

SEQ ID NO: 219 - protein, 6 aa, synthetic construct
  source 1..6: note = R3-P3-C11 HC Contact CDR1
NNYAVH

SEQ ID NO: 220 - protein, 13 aa, synthetic construct
  source 1..13: note = R3-P3-C11 HC Contact CDR2
WVAVISYDGTNKY

SEQ ID NO: 221 - protein, 12 aa, synthetic construct
  source 1..12: note = R3-P3-C11 HC Contact CDR3
ARGNYVRRDSFD

SEQ ID NO: 222 - protein, 7 aa, synthetic construct
  source 1..7: note = R3-P3-C11 LC Contact CDR1
SSYLNWY

SEQ ID NO: 223 - protein, 10 aa, synthetic construct
  source 1..10: note = R3-P3-C11 LC Contact CDR2
LLIYGASSLQ

SEQ ID NO: 224 - protein, 8 aa, synthetic construct
  source 1..8: note = R3-P3-C11 LC Contact CDR3
QQSYISPI

SEQ ID NO: 225 - protein, 8 aa, synthetic construct
  source 1..8: note = R3-P3-C11 HC IMGT CDR1
GFTFNNYA

SEQ ID NO: 226 - protein, 8 aa, synthetic construct
  source 1..8: note = R3-P3-C11 HC IMGT CDR2
ISYDGTNK

SEQ ID NO: 227 - protein, 13 aa, synthetic construct
  source 1..13: note = R3-P3-C11 HC IMGT CDR3
ARGNYVRRDSFDI

SEQ ID NO: 228 - protein, 6 aa, synthetic construct
  source 1..6: note = R3-P3-C11 LC IMGT CDR1
QSISSY

SEQ ID NO: 229 - skipped

SEQ ID NO: 230 - protein, 9 aa, synthetic construct
  source 1..9: note = R3-P3-C11 LC IMGT CDR3
QQSYISPIT

SEQ ID NO: 231 - protein, 120 aa, synthetic construct; identical to SEQ ID NO: 11
  source 1..120: note = R3-P3-C11 IgG1 Kappa VH AA

SEQ ID NO: 232 - protein, 107 aa, synthetic construct; identical to SEQ ID NO: 12
  source 1..107: note = R3-P3-C11 IgG1 Kappa VL AA

SEQ ID NO: 233 - protein, 473 aa, synthetic construct
  source 1..473: note = R3-P3-C11 IgG1 Kappa Heavy Chain AA
MDPKGSLSWRILLFLSLAFELSYGEVQLVESGGGLVQPGRSLRLACEASGFTFNNYAVHWVRQAPGKGLEWVAVISYDGTNKYYSDSMKGRFTISRDNSKNTLYLQINSLRVEDTAVYFCARGNYVRRDSFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 234 - protein, 234 aa, synthetic construct
  source 1..234: note = R3-P3-C11 IgG1 Kappa Light Chain AA
METDTLLLWVLLLWVPGSTGDIQMTQSPPSLSASVGDRVTITCRASQSISSYLNWYQQKPGKAPKLLIYGASSLQSGVPLRFSGSGSGADFTLTISSLQPEDFATYYCQQSYISPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 235 - protein, 5 aa, synthetic construct
  source 1..5: note = R4M-P3-E06 HC Kabat CDR1
NYGMH

SEQ ID NO: 236 - protein, 17 aa, synthetic construct
  source 1..17: note = R4M-P3-E06 HC Kabat CDR2
TISYDGTKKYYPDSVRG

SEQ ID NO: 237 - protein, 14 aa, synthetic construct
  source 1..14: note = R4M-P3-E06 HC Kabat CDR3
DLVSGSRYGFAFDS

SEQ ID NO: 238 - protein, 16 aa, synthetic construct
  source 1..16: note = R4M-P3-E06 LC Kabat CDR1
RSSQSLVHSDGNTYLN

SEQ ID NO: 239 - protein, 7 aa, synthetic construct
  source 1..7: note = R4M-P3-E06 LC Kabat CDR2
KVSNRDS

SEQ ID NO: 240 - protein, 9 aa, synthetic construct
  source 1..9: note = R4M-P3-E06 LC Kabat CDR3
MQGTHWPPI

SEQ ID NO: 241 - protein, 7 aa, synthetic construct
  source 1..7: note = R4M-P3-E06 HC Chothia CDR1
GFTFSNY

SEQ ID NO: 242 - protein, 6 aa, synthetic construct
  source 1..6: note = R4M-P3-E06 HC Chothia CDR2
SYDGTK

SEQ ID NO: 243 - protein, 14 aa, synthetic construct
  source 1..14: note = R4M-P3-E06 HC Chothia CDR3
DLVSGSRYGFAFDS

SEQ ID NO: 244 - protein, 16 aa, synthetic construct
  source 1..16: note = R4M-P3-E06 LC Chothia CDR1
RSSQSLVHSDGNTYLN

SEQ ID NO: 245 - protein, 7 aa, synthetic construct
  source 1..7: note = R4M-P3-E06 LC Chothia CDR2
KVSNRDS

SEQ ID NO: 246 - protein, 9 aa, synthetic construct
  source 1..9: note = R4M-P3-E06 LC Chothia CDR3
MQGTHWPPI

SEQ ID NO: 247 - protein, 10 aa, synthetic construct
  source 1..10: note = R4M-P3-E06 HC AbM CDR1
GFTFSNYGMH

SEQ ID NO: 248 - protein, 10 aa, synthetic construct
  source 1..10: note = R4M-P3-E06 HC AbM CDR2
TISYDGTKKY

SEQ ID NO: 249 - protein, 14 aa, synthetic construct
  source 1..14: note = R4M-P3-E06 HC AbM CDR3
DLVSGSRYGFAFDS

SEQ ID NO: 250 - protein, 16 aa, synthetic construct
  source 1..16: note = R4M-P3-E06 LC AbM CDR1
RSSQSLVHSDGNTYLN

SEQ ID NO: 251 - protein, 7 aa, synthetic construct
  source 1..7: note = R4M-P3-E06 LC AbM CDR2
KVSNRDS

SEQ ID NO: 252 - protein, 9 aa, synthetic construct
  source 1..9: note = R4M-P3-E06 LC AbM CDR3
MQGTHWPPI

SEQ ID NO: 253 - protein, 6 aa, synthetic construct
  source 1..6: note = R4M-P3-E06 HC Contact CDR1
SNYGMH

SEQ ID NO: 254 - protein, 13 aa, synthetic construct
  source 1..13: note = R4M-P3-E06 HC Contact CDR2
WVATISYDGTKKY

SEQ ID NO: 255 - protein, 15 aa, synthetic construct
  source 1..15: note = R4M-P3-E06 HC Contact CDR3
AKDLVSGSRYGFAFD

SEQ ID NO: 256 - protein, 12 aa, synthetic construct
  source 1..12: note = R4M-P3-E06 LC Contact CDR1
VHSDGNTYLNWF

SEQ ID NO: 257 - protein, 10 aa, synthetic construct
  source 1..10: note = R4M-P3-E06 LC Contact CDR2
RLIYKVSNRD

SEQ ID NO: 258 - protein, 8 aa, synthetic construct
  source 1..8: note = R4M-P3-E06 LC Contact CDR3
MQGTHWPP

SEQ ID NO: 259 - protein, 8 aa, synthetic construct
  source 1..8: note = R4M-P3-E06 HC IMGT CDR1
GFTFSNYG

SEQ ID NO: 260 - protein, 8 aa, synthetic construct
  source 1..8: note = R4M-P3-E06 HC IMGT CDR2
ISYDGTKK

SEQ ID NO: 261 - protein, 16 aa, synthetic construct
  source 1..16: note = R4M-P3-E06 HC IMGT CDR3
AKDLVSGSRYGFAFDS

SEQ ID NO: 262 - protein, 11 aa, synthetic construct
  source 1..11: note = R4M-P3-E06 LC IMGT CDR1
QSLVHSDGNTY

SEQ ID NO: 263 - skipped

SEQ ID NO: 264 - protein, 9 aa, synthetic construct
  source 1..9: note = R4M-P3-E06 LC IMGT CDR3
MQGTHWPPI

SEQ ID NO: 265 - protein, 123 aa, synthetic construct; identical to SEQ ID NO: 13
  source 1..123: note = R4M-P3-E06 IgG1 Kappa VH AA

SEQ ID NO: 266 - protein, 112 aa, synthetic construct; identical to SEQ ID NO: 14
  source 1..112: note = R4M-P3-E06 IgG1 Kappa VL AA

SEQ ID NO: 267 - protein, 476 aa, synthetic construct
  source 1..476: note = R4M-P3-E06 IgG1 Kappa Heavy Chain AA
MDPKGSLSWRILLFLSLAFELSYGEVQLVESGGGVVQPGGSLRLSCAASGFTFSNYGMHWVRQAPGKGLEWVATISYDGTKKYYPDSVRGRFTISRDNSENMLYLQMDSLRLEDTAVIYCAKDLVSGSRYGFAFDSWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 268 - protein, 239 aa, synthetic construct
  source 1..239: note = R4M-P3-E06 IgG1 Kappa Light Chain AA
METDTLLLWVLLLWVPGSTGDIVMTQSPLSLPVTLGQPASISCRSSQSLVHSDGNTYLNWFQQRPGQSPRRLIYKVSNRDSGVPDRFSGTGSGTDFTLKISRVEADDVGVYYCMQGTHWPPIFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 269 - protein, 5 aa, synthetic construct
  source 1..5: note = R3-P3-E09 HC Kabat CDR1
NYAMH

SEQ ID NO: 270 - protein, 17 aa, synthetic construct
  source 1..17: note = R3-P3-E09 HC Kabat CDR2
VISYDGTKKYYGDSVKG

SEQ ID NO: 271 - protein, 14 aa, synthetic construct
  source 1..14: note = R3-P3-E09 HC Kabat CDR3
DVGKGVTATGTFDI

SEQ ID NO: 272 - protein, 11 aa, synthetic construct
  source 1..11: note = R3-P3-E09 LC Kabat CDR1
RASQGIGSYLA

SEQ ID NO: 273 - protein, 7 aa, synthetic construct
  source 1..7: note = R3-P3-E09 LC Kabat CDR2
DATRLKS

SEQ ID NO: 274 - protein, 9 aa, synthetic construct
  source 1..9: note = R3-P3-E09 LC Kabat CDR3
QQYNVSPLT

SEQ ID NO: 275 - protein, 7 aa, synthetic construct
  source 1..7: note = R3-P3-E09 HC Chothia CDR1
EFTFSNY

SEQ ID NO: 276 - protein, 6 aa, synthetic construct
  source 1..6: note = R3-P3-E09 HC Chothia CDR2
SYDGTK

SEQ ID NO: 277 - protein, 14 aa, synthetic construct
  source 1..14: note = R3-P3-E09 HC Chothia CDR3
DVGKGVTATGTFDI

SEQ ID NO: 278 - protein, 11 aa, synthetic construct
  source 1..11: note = R3-P3-E09 LC Chothia CDR1
RASQGIGSYLA

SEQ ID NO: 279 - protein, 7 aa, synthetic construct
  source 1..7: note = R3-P3-E09 LC Chothia CDR2
DATRLKS

SEQ ID NO: 280 - protein, 9 aa, synthetic construct
  source 1..9: note = R3-P3-E09 LC Chothia CDR3
QQYNVSPLT

SEQ ID NO: 281 - protein, 10 aa, synthetic construct
  source 1..10: note = R3-P3-E09 HC AbM CDR1
EFTFSNYAMH

SEQ ID NO: 282 - protein, 10 aa, synthetic construct
  source 1..10: note = R3-P3-E09 HC AbM CDR2
VISYDGTKKY

SEQ ID NO: 283 - protein, 14 aa, synthetic construct
  source 1..14: note = R3-P3-E09 HC AbM CDR3
DVGKGVTATGTFDI

SEQ ID NO: 284 - protein, 11 aa, synthetic construct
  source 1..11: note = R3-P3-E09 LC AbM CDR1
RASQGIGSYLA

SEQ ID NO: 285 - protein, 7 aa, synthetic construct
  source 1..7: note = R3-P3-E09 LC AbM CDR2
DATRLKS

SEQ ID NO: 286 - protein, 9 aa, synthetic construct
  source 1..9: note = R3-P3-E09 LC AbM CDR3
QQYNVSPLT

SEQ ID NO: 287 - protein, 6 aa, synthetic construct
  source 1..6: note = R3-P3-E09 HC Contact CDR1
SNYAMH

SEQ ID NO: 288 - protein, 13 aa, synthetic construct
  source 1..13: note = R3-P3-E09 HC Contact CDR2
WVAVISYDGTKKY

SEQ ID NO: 289 - protein, 15 aa, synthetic construct
  source 1..15: note = R3-P3-E09 HC Contact CDR3
ARDVGKGVTATGTFD

SEQ ID NO: 290 - protein, 7 aa, synthetic construct
  source 1..7: note = R3-P3-E09 LC Contact CDR1
GSYLAWF

SEQ ID NO: 291 - protein, 10 aa, synthetic construct
  source 1..10: note = R3-P3-E09 LC Contact CDR2
SLIIDATRLK

SEQ ID NO: 292 - protein, 8 aa, synthetic construct
  source 1..8: note = R3-P3-E09 LC Contact CDR3
QQYNVSPL

SEQ ID NO: 293 - protein, 8 aa, synthetic construct
  source 1..8: note = R3-P3-E09 HC IMGT CDR1
EFTFSNYA

SEQ ID NO: 294 - protein, 8 aa, synthetic construct
  source 1..8: note = R3-P3-E09 HC IMGT CDR2
ISYDGTKK

SEQ ID NO: 295 - protein, 16 aa, synthetic construct
  source 1..16: note = R3-P3-E09 HC IMGT CDR3
ARDVGKGVTATGTFDI

SEQ ID NO: 296 - protein, 6 aa, synthetic construct
  source 1..6: note = R3-P3-E09 LC IMGT CDR1
QGIGSY

SEQ ID NO: 297 - skipped

SEQ ID NO: 298 - protein, 9 aa, synthetic construct
  source 1..9: note = R3-P3-E09 LC IMGT CDR3
QQYNVSPLT

SEQ ID NO: 299 - protein, 123 aa, synthetic construct; identical to SEQ ID NO: 15
  source 1..123: note = R3-P3-E09 IgG1 Kappa VH AA

SEQ ID NO: 300 - protein, 107 aa, synthetic construct; identical to SEQ ID NO: 16
  source 1..107: note = R3-P3-E09 IgG1 Kappa VL AA

SEQ ID NO: 301 - protein, 476 aa, synthetic construct
  source 1..476: note = R3-P3-E09 IgG1 Kappa Heavy Chain AA
MDPKGSLSWRILLFLSLAFELSYGEVQLVETGGGVVQPGRSLTLSCGASEFTFSNYAMHWVRQAPGKGLEWVAVISYDGTKKYYGDSVKGRFTISRENIKNTLYLQMNSLRSEDTAVYYCARDVGKGVTATGTFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 302 - protein, 234 aa, synthetic construct
  source 1..234: note = R3-P3-E09 IgG1 Kappa Light Chain AA
METDTLLLWVLLLWVPGSTGAIRMTQSPSSLSASVGDRVTITCRASQGIGSYLAWFQQKPGEAPKSLIIDATRLKSGVPSRFSGSGSGTEFALTISSLRPEDFATYYCQQYNVSPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 303 - protein, 5 aa, synthetic construct
  source 1..5: note = R3-P1-C02 HC Kabat CDR1
DFGMH

SEQ ID NO: 304 - protein, 17 aa, synthetic construct
  source 1..17: note = R3-P1-C02 HC Kabat CDR2
FVRSHGSGKYYADSVKG

SEQ ID NO: 305 - protein, 4 aa, synthetic construct
  source 1..4: note = R3-P1-C02 HC Kabat CDR3
LADV

SEQ ID NO: 306 - protein, 16 aa, synthetic construct
  source 1..16: note = R3-P1-C02 LC Kabat CDR1
RSSQSLVYSDGNTYLN

SEQ ID NO: 307 - protein, 7 aa, synthetic construct
  source 1..7: note = R3-P1-C02 LC Kabat CDR2
KVSNRDS

SEQ ID NO: 308 - protein, 9 aa, synthetic construct
  source 1..9: note = R3-P1-C02 LC Kabat CDR3
MQGTHWPWT

SEQ ID NO: 309 - protein, 7 aa, synthetic construct
  source 1..7: note = R3-P1-C02 HC Chothia CDR1
EFPFSDF

SEQ ID NO: 310 - protein, 6 aa, synthetic construct
  source 1..6: note = R3-P1-C02 HC Chothia CDR2
RSHGSG

SEQ ID NO: 311 - protein, 4 aa, synthetic construct
  source 1..4: note = R3-P1-C02 HC Chothia CDR3
LADV

SEQ ID NO: 312 - protein, 16 aa, synthetic construct
  source 1..16: note = R3-P1-C02 LC Chothia CDR1
RSSQSLVYSDGNTYLN

SEQ ID NO: 313 - protein, 7 aa, synthetic construct
  source 1..7: note = R3-P1-C02 LC Chothia CDR2
KVSNRDS

SEQ ID NO: 314 - protein, 9 aa, synthetic construct
  source 1..9: note = R3-P1-C02 LC Chothia CDR3
MQGTHWPWT

SEQ ID NO: 315 - protein, 10 aa, synthetic construct
  source 1..10: note = R3-P1-C02 HC AbM CDR1
EFPFSDFGMH

SEQ ID NO: 316 - protein, 10 aa, synthetic construct
  source 1..10: note = R3-P1-C02 HC AbM CDR2
FVRSHGSGKY

SEQ ID NO: 317 - protein, 4 aa, synthetic construct
  source 1..4: note = R3-P1-C02 HC AbM CDR3
LADV

SEQ ID NO: 318 - protein, 16 aa, synthetic construct
  source 1..16: note = R3-P1-C02 LC AbM CDR1
RSSQSLVYSDGNTYLN

SEQ ID NO: 319 - protein, 9 aa, synthetic construct
  source 1..9: note = R3-P1-C02 LC AbM CDR2
MQGTHWPWT

SEQ ID NO: 320 - skipped

SEQ ID NO: 321 - protein, 6 aa, synthetic construct
  source 1..6: note = R3-P1-C02 HC Contact CDR1
SDFGMH

SEQ ID NO: 322 - protein, 13 aa, synthetic construct
  source 1..13: note = R3-P1-C02 HC Contact CDR2
WVAFVRSHGSGKY

SEQ ID NO: 323 - protein, 5 aa, synthetic construct
  source 1..5: note = R3-P1-C02 HC Contact CDR3
TTLAD

SEQ ID NO: 324 - protein, 12 aa, synthetic construct
  source 1..12: note = R3-P1-C02 LC Contact CDR1
VYSDGNTYLNWF

SEQ ID NO: 325 - protein, 10 aa, synthetic construct
  source 1..10: note = R3-P1-C02 LC Contact CDR2
RLIYKVSNRD

SEQ ID NO: 326 - protein, 8 aa, synthetic construct
  source 1..8: note = R3-P1-C02 LC Contact CDR3
MQGTHWPW

SEQ ID NO: 327 - protein, 8 aa, synthetic construct
  source 1..8: note = R3-P1-C02 HC IMGT CDR1
EFPFSDFG

SEQ ID NO: 328 - protein, 8 aa, synthetic construct
  source 1..8: note = R3-P1-C02 HC IMGT CDR2
VRSHGSGK

SEQ ID NO: 329 - protein, 6 aa, synthetic construct
  source 1..6: note = R3-P1-C02 HC IMGT CDR3
TTLADV

SEQ ID NO: 330 - protein, 11 aa, synthetic construct
  source 1..11: note = R3-P1-C02 LC IMGT CDR1
QSLVYSDGNTY

SEQ ID NO: 331 - skipped

SEQ ID NO: 332 - protein, 9 aa, synthetic construct
  source 1..9: note = R3-P1-C02 LC IMGT CDR3
MQGTHWPWT

SEQ ID NO: 333 - protein, 113 aa, synthetic construct; identical to SEQ ID NO: 17
  source 1..113: note = R3-P1-C02 IgG1 Kappa VH AA

SEQ ID NO: 334 - protein, 112 aa, synthetic construct; identical to SEQ ID NO: 18
  source 1..112: note = R3-P1-C02 IgG1 Kappa VL AA

SEQ ID NO: 335 - protein, 466 aa, synthetic construct
  source 1..466: note = R3-P1-C02 IgG1 Kappa Heavy Chain AA
MDPKGSLSWRILLFLSLAFELSYGEVQLVESGGGVVQPGGSLRISCVASEFPFSDFGMHWVRQAPGKGLEWVAFVRSHGSGKYYADSVKGRFTISGDKSKSTLYLQMESLRPDDAAVYYCTTLADVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 336 - protein, 239 aa, synthetic construct
  source 1..239: note = R3-P1-C02 IgG1 Kappa Light Chain AA
METDTLLLWVLLLWVPGSTGDVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 337 - protein, 5 aa, synthetic construct
  source 1..5: note = R3-P3-A12 HC Kabat CDR1
SFGMH

SEQ ID NO: 338 - protein, 17 aa, synthetic construct
  source 1..17: note = R3-P3-A12 HC Kabat CDR2
VISNDALHKNYGDSVKG

SEQ ID NO: 339 - protein, 13 aa, synthetic construct
  source 1..13: note = R3-P3-A12 HC Kabat CDR3
DVVRGHTSGSLGS

SEQ ID NO: 340 - protein, 16 aa, synthetic construct
  source 1..16: note = R3-P3-A12 LC Kabat CDR1
RSSQSLLYSNGYNYLD

SEQ ID NO: 341 - protein, 7 aa, synthetic construct
  source 1..7: note = R3-P3-A12 LC Kabat CDR2
LGSNRAS

SEQ ID NO: 342 - protein, 9 aa, synthetic construct
  source 1..9: note = R3-P3-A12 LC Kabat CDR3
MQALQTPLT

SEQ ID NO: 343 - protein, 7 aa, synthetic construct
  source 1..7: note = R3-P3-A12 HC Chothia CDR1
GFTFSSF

SEQ ID NO: 344 - protein, 6 aa, synthetic construct
  source 1..6: note = R3-P3-A12 HC Chothia CDR2
SNDALH

SEQ ID NO: 345 - protein, 13 aa, synthetic construct
  source 1..13: note = R3-P3-A12 HC Chothia CDR3
DVVRGHTSGSLGS

SEQ ID NO: 346 - protein, 16 aa, synthetic construct
  source 1..16: note = R3-P3-A12 LC Chothia CDR1
RSSQSLLYSNGYNYLD

SEQ ID NO: 347 - protein, 7 aa, synthetic construct
  source 1..7: note = R3-P3-A12 LC Chothia CDR2
LGSNRAS

SEQ ID NO: 348 - protein, 9 aa, synthetic construct
  source 1..9: note = R3-P3-A12 LC Chothia CDR3
MQALQTPLT

SEQ ID NO: 349 - protein, 10 aa, synthetic construct
  source 1..10: note = R3-P3-A12 HC AbM CDR1
GFTFSSFGMH

SEQ ID NO: 350 - protein, 10 aa, synthetic construct
  source 1..10: note = R3-P3-A12 HC AbM CDR2
VISNDALHKN

SEQ ID NO: 351 - protein, 13 aa, synthetic construct
  source 1..13: note = R3-P3-A12 HC AbM CDR3
DVVRGHTSGSLGS

SEQ ID NO: 352 - protein, 16 aa, synthetic construct
  source 1..16: note = R3-P3-A12 LC AbM CDR1
RSSQSLLYSNGYNYLD

SEQ ID NO: 353 - protein, 7 aa, synthetic construct
  source 1..7: note = R3-P3-A12 LC AbM CDR2
LGSNRAS

SEQ ID NO: 354 - protein, 9 aa, synthetic construct
  source 1..9: note = R3-P3-A12 LC AbM CDR3
MQALQTPLT

SEQ ID NO: 355 - protein, 6 aa, synthetic construct
  source 1..6: note = R3-P3-A12 HC Contact CDR1
SSFGMH

SEQ ID NO: 356 - protein, 13 aa, synthetic construct
  source 1..13: note = R3-P3-A12 HC Contact CDR2
WVAVISNDALHKN

SEQ ID NO: 357 - protein, 14 aa, synthetic construct
  source 1..14: note = R3-P3-A12 HC Contact CDR3
AKDVVRGHTSGSLG

SEQ ID NO: 358 - protein, 12 aa, synthetic construct
  source 1..12: note = R3-P3-A12 LC Contact CDR1
LYSNGYNYLDWY

SEQ ID NO: 359 - protein, 10 aa, synthetic construct
  source 1..10: note = R3-P3-A12 LC Contact CDR2
LLIYLGSNRA

SEQ ID NO: 360 - protein, 8 aa, synthetic construct
  source 1..8: note = R3-P3-A12 LC Contact CDR3
MQALQTPL

SEQ ID NO: 361 - protein, 8 aa, synthetic construct
  source 1..8: note = R3-P3-A12 HC IMGT CDR1
GFTFSSFG

SEQ ID NO: 362 - protein, 8 aa, synthetic construct
  source 1..8: note = R3-P3-A12 HC IMGT CDR2
ISNDALHK

SEQ ID NO: 363 - protein, 15 aa, synthetic construct
  source 1..15: note = R3-P3-A12 HC IMGT CDR3
AKDVVRGHTSGSLGS

SEQ ID NO: 364 - protein, 11 aa, synthetic construct
  source 1..11: note = R3-P3-A12 LC IMGT CDR1
QSLLYSNGYNY

SEQ ID NO: 365 - skipped

SEQ ID NO: 366 - protein, 9 aa, synthetic construct
  source 1..9: note = R3-P3-A12 LC IMGT CDR3
MQALQTPLT

SEQ ID NO: 367 - protein, 122 aa, synthetic construct; identical to SEQ ID NO: 19
  source 1..122: note = R3-P3-A12 IgG1 Kappa VH AA

SEQ ID NO: 368 - protein, 112 aa, synthetic construct; identical to SEQ ID NO: 20
  source 1..112: note = R3-P3-A12 IgG1 Kappa VL AA

SEQ ID NO: 369 - protein, 475 aa, synthetic construct
  source 1..475: note = R3-P3-A12 IgG1 Kappa Heavy Chain AA
MDPKGSLSWRILLFLSLAFELSYGEVQLVESGGGLVQPGRSLRLSCGVSGFTFSSFGMHWVRQAPGKGLEWVAVISNDALHKNYGDSVKGRFTVTRDNSRNTLFLQMTSLRPEDTAVYYCAKDVVRGHTSGSLGSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 370 - protein, 239 aa, synthetic construct
  source 1..239: note = R3-P3-A12 IgG1 Kappa Light Chain AA
METDTLLLWVLLLWVPGSTGDVVMTQSPLSLPVTPGEPASISCRSSQSLLYSNGYNYLDWYLQKPGQSPQLLIYLGSNRASGVPDRFSGSGSGTDFTLKISRVEAEDVGVYYCMQALQTPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 371 - protein, 5 aa, synthetic construct
  source 1..5: note = R4M-P3-A12 HC Kabat CDR1
DHYMS

SEQ ID NO: 372 - protein, 17 aa, synthetic construct
  source 1..17: note = R4M-P3-A12 HC Kabat CDR2
YLSGSGTSIYYADSVKG

SEQ ID NO: 373 - protein, 11 aa, synthetic construct
  source 1..11: note = R4M-P3-A12 HC Kabat CDR3
VGPAAGNAFDI

SEQ ID NO: 374 - protein, 13 aa, synthetic construct
  source 1..13: note = R4M-P3-A12 LC Kabat CDR1
SGSSSNIGSNYVY

SEQ ID NO: 375 - protein, 7 aa, synthetic construct
  source 1..7: note = R4M-P3-A12 LC Kabat CDR2
RNNQRPS

SEQ ID NO: 376 - protein, 11 aa, synthetic construct
  source 1..11: note = R4M-P3-A12 LC Kabat CDR3
QSYDSSLSGVV

SEQ ID NO: 377 - protein, 7 aa, synthetic construct
  source 1..7: note = R4M-P3-A12 HC Chothia CDR1
GFTFSDH

SEQ ID NO: 378 - protein, 6 aa, synthetic construct
  source 1..6: note = R4M-P3-A12 HC Chothia CDR2
SGSGTS

SEQ ID NO: 379 - protein, 11 aa, synthetic construct
  source 1..11: note = R4M-P3-A12 HC Chothia CDR3
VGPAAGNAFDI

SEQ ID NO: 380 - protein, 13 aa, synthetic construct
  source 1..13: note = R4M-P3-A12 LC Chothia CDR1
SGSSSNIGSNYVY

SEQ ID NO: 381 - protein, 7 aa, synthetic construct
  source 1..7: note = R4M-P3-A12 LC Chothia CDR2
RNNQRPS

SEQ ID NO: 382 - protein, 11 aa, synthetic construct
  source 1..11: note = R4M-P3-A12 LC Chothia CDR3
QSYDSSLSGVV

SEQ ID NO: 383 - protein, 10 aa, synthetic construct
  source 1..10: note = R4M-P3-A12 HC AbM CDR1
GFTFSDHYMS

SEQ ID NO: 384 - protein, 10 aa, synthetic construct
  source 1..10: note = R4M-P3-A12 HC AbM CDR2
YLSGSGTSIY

SEQ ID NO: 385 - protein, 11 aa, synthetic construct
  source 1..11: note = R4M-P3-A12 HC AbM CDR3
VGPAAGNAFDI

SEQ ID NO: 386 - protein, 13 aa, synthetic construct
  source 1..13: note = R4M-P3-A12 LC AbM CDR1
SGSSSNIGSNYVY

SEQ ID NO: 387 - protein, 7 aa, synthetic construct
  source 1..7: note = R4M-P3-A12 LC AbM CDR2
RNNQRPS

SEQ ID NO: 388 - protein, 11 aa, synthetic construct
  source 1..11: note = R4M-P3-A12 LC AbM CDR3
QSYDSSLSGVV

SEQ ID NO: 389 - protein, 6 aa, synthetic construct
  source 1..6: note = R4M-P3-A12 HC Contact CDR1
SDHYMS

SEQ ID NO: 390 - protein, 13 aa, synthetic construct
  source 1..13: note = R4M-P3-A12 HC Contact CDR2
WVSYLSGSGTSIY

SEQ ID NO: 391 - protein, 12 aa, synthetic construct
  source 1..12: note = R4M-P3-A12 HC Contact CDR3
ARVGPAAGNAFD

SEQ ID NO: 392 - protein, 9 aa, synthetic construct
  source 1..9: note = R4M-P3-A12 LC Contact CDR1
IGSNYVYWY

SEQ ID NO: 393 - protein, 10 aa, synthetic construct
  source 1..10: note = R4M-P3-A12 LC Contact CDR2
LLIYRNNQRP

SEQ ID NO: 394 - protein, 10 aa, synthetic construct
  source 1..10: note = R4M-P3-A12 LC Contact CDR3
QSYDSSLSGV

SEQ ID NO: 395 - protein, 8 aa, synthetic construct
  source 1..8: note = R4M-P3-A12 HC IMGT CDR1
GFTFSDHY

SEQ ID NO: 396 - protein, 8 aa, synthetic construct
  source 1..8: note = R4M-P3-A12 HC IMGT CDR2
LSGSGTSI

SEQ ID NO: 397 - protein, 13 aa, synthetic construct
  source 1..13: note = R4M-P3-A12 HC IMGT CDR3
ARVGPAAGNAFDI

SEQ ID NO: 398 - protein, 8 aa, synthetic construct
  source 1..8: note = R4M-P3-A12 LC IMGT CDR1
SSNIGSNY

SEQ ID NO: 399 - skipped

SEQ ID NO: 400 - protein, 11 aa, synthetic construct
  source 1..11: note = R4M-P3-A12 LC IMGT CDR3
QSYDSSLSGVV

SEQ ID NO: 401 - protein, 120 aa, synthetic construct; identical to SEQ ID NO: 21
  source 1..120: note = R4M-P3-A12 IgG1 Lamba3 VH AA

SEQ ID NO: 402 - protein, 110 aa, synthetic construct; identical to SEQ ID NO: 22
  source 1..110: note = R4M-P3-A12 IgG1 Lamba3 VL AA

SEQ ID NO: 403 - protein, 473 aa, synthetic construct
  source 1..473: note = R4M-P3-A12 IgG1 Lamba3 Heavy Chain AA
MDPKGSLSWRILLFLSLAFELSYGEVQLVESGGGVVQPGGSLRLSCAASGFTFSDHYMSWIRQAPGKGLEWVSYLSGSGTSIYYADSVKGRFTISRDNAKKSLYLQMNSLRAEDTAVYYCARVGPAAGNAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 404 - protein, 237 aa, synthetic construct
  source 1..237: note = R4M-P3-A12 IgG1 Lamba3 Light Chain AA
METDTLLLWVLLLWVPGSTGQSVLTQPPSASGTPGQRVTISCSGSSSNIGSNYVYWYQQLPGTAPKLLIYRNNQRPSGVPDRFSGSKSGTSASLAITGLQAEDEADYYCQSYDSSLSGVVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 405 - protein, 5 aa, synthetic construct
  source 1..5: note = R4M-P1-A10 HC Kabat CDR1
SYAMH

SEQ ID NO: 406 - protein, 17 aa, synthetic construct
  source 1..17: note = R4M-P1-A10 HC Kabat CDR2
VISYDGSNKYYADSVKG

SEQ ID NO: 407 - protein, 10 aa, synthetic construct
  source 1..10: note = R4M-P1-A10 HC Kabat CDR3
DRGWADAFDI

SEQ ID NO: 408-410 - skipped (3 entries)

SEQ ID NO: 411 - protein, 7 aa, synthetic construct
  source 1..7: note = R4M-P1-A10 HC Chothia CDR1
GFTFSSY

SEQ ID NO: 412 - protein, 6 aa, synthetic construct
  source 1..6: note = R4M-P1-A10 HC Chothia CDR2
SYDGSN

SEQ ID NO: 413 - protein, 10 aa, synthetic construct
  source 1..10: note = R4M-P1-A10 HC Chothia CDR3
DRGWADAFDI

SEQ ID NO: 414 - protein, 8 aa, synthetic construct
  source 1..8: note = R4M-P1-A10 LC Chothia CDR1
SNNVGYQG

SEQ ID NO: 415 - skipped

SEQ ID NO: 416 - protein, 11 aa, synthetic construct
  source 1..11: note = R4M-P1-A10 LC Chothia CDR3
AAWDGSLRGWV

SEQ ID NO: 417 - protein, 10 aa, synthetic construct
  source 1..10: note = R4M-P1-A10 HC AbM CDR1
GFTFSSYAMH

SEQ ID NO: 418 - protein, 10 aa, synthetic construct
  source 1..10: note = R4M-P1-A10 HC AbM CDR2
VISYDGSNKY

SEQ ID NO: 419 - protein, 10 aa, synthetic construct
  source 1..10: note = R4M-P1-A10 HC AbM CDR3
DRGWADAFDI

SEQ ID NO: 420 - protein, 8 aa, synthetic construct
  source 1..8: note = R4M-P1-A10 LC AbM CDR1
SNNVGYQG

SEQ ID NO: 421 - skipped

SEQ ID NO: 422 - protein, 11 aa, synthetic construct
  source 1..11: note = R4M-P1-A10 LC AbM CDR3
AAWDGSLRGWV

SEQ ID NO: 423 - protein, 6 aa, synthetic construct
  source 1..6: note = R4M-P1-A10 HC Contact CDR1
SSYAMH

SEQ ID NO: 424 - protein, 13 aa, synthetic construct
  source 1..13: note = R4M-P1-A10 HC Contact CDR2
WVAVISYDGSNKY

SEQ ID NO: 425 - protein, 11 aa, synthetic construct
  source 1..11: note = R4M-P1-A10 HC Contact CDR3
ARDRGWADAFD

SEQ ID NO: 426 - protein, 8 aa, synthetic construct
  source 1..8: note = R4M-P1-A10 LC Contact CDR1
SNNVGYQG

SEQ ID NO: 427 - skipped

SEQ ID NO: 428 - protein, 11 aa, synthetic construct
  source 1..11: note = R4M-P1-A10 LC Contact CDR3
AAWDGSLRGWV

SEQ ID NO: 429 - protein, 8 aa, synthetic construct
  source 1..8: note = R4M-P1-A10 HC IMGT CDR1
GFTFSSYA

SEQ ID NO: 430 - protein, 8 aa, synthetic construct
  source 1..8: note = R4M-P1-A10 HC IMGT CDR2
ISYDGSNK

SEQ ID NO: 431 - protein, 12 aa, synthetic construct
  source 1..12: note = R4M-P1-A10 HC IMGT CDR3
ARDRGWADAFDI

SEQ ID NO: 432 - protein, 8 aa, synthetic construct
  source 1..8: note = R4M-P1-A10 LC IMGT CDR1
SNNVGYQG

SEQ ID NO: 433 - skipped

SEQ ID NO: 434 - protein, 11 aa, synthetic construct
  source 1..11: note = R4M-P1-A10 LC IMGT CDR3
AAWDGSLRGWV

SEQ ID NO: 435 - protein, 119 aa, synthetic construct; identical to SEQ ID NO: 23
  source 1..119: note = R4M-P1-A10 IgG1 Lamba3 VH AA

SEQ ID NO: 436 - protein, 110 aa, synthetic construct; identical to SEQ ID NO: 24
  source 1..110: note = R4M-P1-A10 IgG1 Lamba3 VL AA

SEQ ID NO: 437 - protein, 472 aa, synthetic construct
  source 1..472: note = R4M-P1-A10 IgG1 Lamba3 Heavy Chain AA
MDPKGSLSWRILLFLSLAFELSYGEVQLVETGGGVVQPGRSLRLSCAASGFTFSSYAMHWVRQAPGKGLEWVAVISYDGSNKYYADSVKGRFTISRDNSKNTLYLQMNSLRAEDTAVYYCARDRGWADAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 438 - protein, 237 aa, synthetic construct
  source 1..237: note = R4M-P1-A10 IgG1 Lamba3 Light Chain AA
METDTLLLWVLLLWVPGSTGQAGLTQPPSVSKGLRQTATLTCTGNSNNVGYQGAVWLLQYQGHPPKVLSNRNNDRPSGISERLSASRSGNTASLTITGLQPEDEGDYFCAAWDGSLRGWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 439 - protein, 1337 aa, Homo sapiens
  source 1..1337: note = exemplary human Dep-1
MKPAAREARLPPRSPGLRWALPLLLLLLRLGQILCAGGTPSPIPDPSVATVATGENGITQISSTAESFHKQNGTGTPQVETNTSEDGESSGANDSLRTPEQGSNGTDGASQKTPSSTGPSPVFDIKAVSISPTNVILTWKSNDTAASEYKYVVKHKMENEKTITVVHQPWCNITGLRPATSYVFSITPGIGNETWGDPRVIKVITEPIPVSDLRVALTGVRKAALSWSNGNGTASCRVLLESIGSHEELTQDSRLQVNISGLKPGVQYNINPYLLQSNKTKGDPLGTEGGLDASNTERSRAGSPTAPVHDESLVGPVDPSSGQQSRDTEVLLVGLEPGTRYNATVYSQAANGTEGQPQAIEFRTNAIQVFDVTAVNISATSLTLIWKVSDNESSSNYTYKIHVAGETDSSNLNVSEPRAVIPGLRSSTFYNITVCPVLGDIEGTPGFLQVHTPPVPVSDFRVTVVSTTEIGLAWSSHDAESFQMHITQEGAGNSRVEITTNQSIIIGGLFPGTKYCFEIVPKGPNGTEGASRTVCNRTVPSAVFDIHVVYVTTTEMWLDWKSPDGASEYVYHLVIESKHGSNHTSTYDKAITLQGLIPGTLYNITISPEVDHVWGDPNSTAQYTRPSNVSNIDVSTNTTAATLSWQNFDDASPTYSYCLLIEKAGNSSNATQVVTDIGITDATVTELIPGSSYTVEIFAQVGDGIKSLEPGRKSFCTDPASMASFDCEVVPKEPALVLKWTCPPGANAGFELEVSSGAWNNATHLESCSSENGTEYRTEVTYLNFSTSYNISITTVSCGKMAAPTRNTCTTGITDPPPPDGSPNITSVSHNSVKVKFSGFEASHGPIKAYAVILTTGEAGHPSADVLKYTYEDFKKGASDTYVTYLIRTEEKGRSQSLSEVLKYEIDVGNESTTLGYYNGKLEPLGSYRACVAGFTNITFHPQNKGLIDGAESYVSFSRYSDAVSLPQDPGVICGAVFGCIFGALVIVTVGGFIFWRKKRKDAKNNEVSFSQIKPKKSKLIRVENFEAYFKKQQADSNCGFAEEYEDLKLVGISQPKYAAELAENRGKNRYNNVLPYDISRVKLSVQTHSTDDYINANYMPGYHSKKDFIATQGPLPNTLKDFWRMVWEKNVYAIIMLTKCVEQGRTKCEEYWPSKQAQDYGDITVAMTSEIVLPEWTIRDFTVKNIQTSESHPLRQFHFTSWPDHGVPDTTDLLINFRYLVRDYMKQSPPESPILVHCSAGVGRTGTFIAIDRLIYQIENENTVDVYGIVYDLRMHRPLMVQTEDQYVFLNQCVLDIVRSQKDSKVDLIYQNTTAMTIYENLAPVTTFGKTNGYIA

(19) United States
(12) Patent Application Publication
(10) Pub. No.: US 2025/0282885 A1
(43) Pub. Date: Sep. 11, 2025

(54) COMPOSITIONS AND METHODS FOR SELECTIVE REGULATION OF VASCULAR PERMEABILITY

(71) Applicant: VST BIO CORPORATION, San Diego, CA (US)

(72) Inventors: Krisztina ZSEBO, Santa Barbara, CA (US); Henricus J. DUCKERS, New York, NY (US)

(21) Appl. No.: 18/862,505

(22) PCT Filed: May 3, 2023
(86) PCT No.: PCT/US2023/066549

§ 371 Date: Nov. 1, 2024

Related U.S. Application Data
(60) Provisional application No. 63/338,359, filed on May 4, 2022.
(60) Provisional application No. 63/444,521, filed on Feb. 9, 2023.
(60) Provisional application No. 63/444,520, filed on Feb. 9, 2023.
(60) Provisional application No. 63/495,765, filed on Apr. 12, 2023.
(60) Provisional application No. 63/495,763, filed on Apr. 12, 2023.

(51) Int. Cl. C07K 16/28; A61K 39/00; A61P 9/10; A61P 27/02; A61P 29/00

(52) U.S. Cl. CPC C07K 16/2896; A61P 9/10; A61P 27/02; A61P 29/00; A61K 2039/505; C07K 2317/24; C07K 2317/31; C07K 2317/35; C07K 2317/565

(57) ABSTRACT

Provided are anti-syndecan-2 antibodies or antigen binding fragments thereof, as well as compositions comprising said antibody or antigen binding fragment thereof and methods useful for treating diseases associated with syndecan-2 mediated vascular permeability.

### CROSS-REFERENCE TO RELATED APPLICATIONS

This application claims the benefit of priority to U.S. Ser. No. 63/338,359 filed May 4, 2022, U.S. Ser. No. 63/444,520 filed Feb. 9, 2023, U.S. Ser. No. 63/444,521 filed Feb. 9, 2023, U.S. Ser. No. 63/495,763 filed Apr. 12, 2023, and U.S. Ser. No. 63/495,765 filed Apr. 12, 2023, each of which is incorporated herein by reference in its entirety.

### 1. SEQUENCE LISTING

This application contains a computer readable Sequence Listing which has been submitted in XML file format with this application, the entire content of which is incorporated by reference herein in its entirety. The Sequence Listing XML file submitted with this application entitled “14765-010-228_SEQLISTING.xml” was created on May 3, 2023 and is 463,326 bytes in size.

### 2. FIELD

Provided herein, in certain aspects, are antibodies that bind to Sdc2, nucleic acids encoding the antibodies, vectors comprising the nucleic acids, as well as recombinant cells containing the vectors, and compositions comprising the antibodies. Methods of making and using the antibodies are also provided.

### 3. BACKGROUND

Vascular leakage associated with inflammation and tissue injury is an important factor in a wide variety of pathologies. Syndecan-2 (also known as Sdc2, SDC2, Sdc-2, and CD362) is a plasma membrane proteoglycan expressed by endothelial cells and neurons and has a protein tyrosine phosphatase (density-enhanced phosphatase) DEP1-binding site on its extracellular domain. Sdc2 plays a significant role in regulating vascular permeability. Previous studies have demonstrated that Sdc2 knockout mice exhibit reduced vascular leakage after stimulation of vascular endothelial growth factor (VEGF) signaling and polyclonal antibodies against mouse Sdc2 likewise reduce vascular leakage in vivo. There is a need in the art for methods and compositions that inhibit Sdc2 signaling, thereby treating vascular leakage, including acute respiratory distress syndrome (ARDS), stroke (e.g., ischemic stroke), inflammatory eye disorders (e.g., age-related macular degeneration (AMD)), cardiovascular disorders (e.g., acute myocardial infarction (AMI)), and other disorders. The present disclosure addresses this need.

### 4. SUMMARY

Provided herein, in certain aspects, are antibodies that bind to Sdc2, as well as methods of use thereof.

In some embodiments, the antibody that binds to Sdc2 is antibody clone 20-H19-AB. In certain embodiments, the antibody comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:61; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:62. In some embodiments, the antibody comprises (i) a VH comprising a VH CDR1, a VH CDR2, and a VH CDR3 having an amino acid sequence of SEQ ID NOs: 31, 32 and 33, respectively; and (ii) a VL comprising a VL CDR1, a VL CDR2, and a VL CDR3 having an amino acid sequence of SEQ ID NOs: 34, 35 and 36, respectively. In some embodiments, the antibody comprises (i) a VH comprising a VH CDR1, a VH CDR2, and a VH CDR3 having an amino acid sequence of SEQ ID NOs: 37, 38 and 39, respectively; and (ii) a VL comprising a VL CDR1, a VL CDR2, and a VL CDR3 having an amino acid sequence of SEQ ID NOs: 40, 41 and 42, respectively. In some embodiments, the antibody comprises (i) a VH comprising a VH CDR1, a VH CDR2, and a VH CDR3 having an amino acid sequence of SEQ ID NOs: 43, 44 and 45, respectively; and (ii) a VL comprising a VL CDR1, a VL CDR2, and a VL CDR3 having an amino acid sequence of SEQ ID NOs: 46, 47 and 48, respectively. In some embodiments, the antibody comprises (i) a VH comprising a VH CDR1, a VH CDR2, and a VH CDR3 having an amino acid sequence of SEQ ID NOs: 49, 50 and 51, respectively; and (ii) a VL comprising a VL CDR1, a VL CDR2, and a VL CDR3 having an amino acid sequence of SEQ ID NOs: 52, 53 and 54, respectively. In some embodiments, the antibody comprises (i) a VH comprising a VH CDR1, a VH CDR2, and a VH CDR3 having an amino acid sequence of SEQ ID NOs: 55, 56 and 57, respectively; and (ii) a VL comprising a VL CDR1, a VL CDR2, and a VL CDR3 having an amino acid sequence of SEQ ID NOs: 58, 59 and 60, respectively. In one embodiment, the antibody comprises a VH having an amino acid sequence having at least 95% identity to an amino acid sequence of SEQ ID NO:61. In another embodiment, the antibody comprises a VL having an amino acid sequence having at least 95% identity to an amino acid sequence of SEQ ID NO: 62. In one embodiment, the antibody comprises (i) a VH having an amino acid sequence having at least 95% identity to an amino acid sequence of SEQ ID NO:61; and (ii) a VL having an amino acid sequence having at least 95% identity to an amino acid sequence of SEQ ID NO:62. In one embodiment, the antibody comprises a VH having an amino acid sequence SEQ ID NO:61. In another embodiment, the antibody comprises a VL having an amino acid sequence SEQ ID NO:62. In one embodiment, the antibody comprises (i) a VH having an amino acid sequence SEQ ID NO:61; and (ii) a VL having an amino acid sequence SEQ ID NO:62. In one embodiment, the antibody comprises a heavy chain having an amino acid sequence having at least 95% identity to an amino acid sequence of SEQ ID NO:63. In another embodiment, the antibody comprises a light chain having an amino acid sequence having at least 95% identity to an amino acid sequence of SEQ ID NO:64. In one embodiment, the antibody comprises (i) a heavy chain having an amino acid sequence having at least 95% identity to an amino acid sequence of SEQ ID NO:63; and (ii) a light chain having an amino acid sequence having at least 95% identity to an amino acid sequence of SEQ ID NO:64. In one embodiment, the antibody comprises a heavy chain having an amino acid sequence SEQ ID NO:63. In another embodiment, the antibody comprises a light chain having an amino acid sequence SEQ ID NO:64. In one embodiment, the antibody comprises (i) a heavy chain having an amino acid sequence SEQ ID NO:63; and (ii) a light chain having an amino acid sequence SEQ ID NO:64.

In some embodiments, the antibody that binds to Sdc2 is antibody clone TP-43327F. In certain embodiments, the antibody comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:95; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:96. In some embodiments, the antibody comprises (i) a VH comprising a VH CDR1, a VH CDR2, and a VH CDR3 having an amino acid sequence of SEQ ID NOs: 65, 66 and 67, respectively; and (ii) a VL comprising a VL CDR1, a VL CDR2, and a VL CDR3 having an amino acid sequence of SEQ ID NOs: 68, 89 and 70, respectively. In other embodiments, the antibody comprises (i) a VH comprising a VH CDR1, a VH CDR2, and a VH CDR3 having an amino acid sequence of SEQ ID NOs: 71, 72 and 73, respectively; and (ii) a VL comprising a VL CDR1, a VL CDR2, and a VL CDR3 having an amino acid sequence of SEQ ID NOs: 74, 75 and 76, respectively. In some embodiments, the antibody comprises (i) a VH comprising a VH CDR1, a VH CDR2, and a VH CDR3 having an amino acid sequence of SEQ ID NOs: 77, 78 and 79, respectively; and (ii) a VL comprising a VL CDR1, a VL CDR2, and a VL CDR3 having an amino acid sequence of SEQ ID NOs: 80, 81 and 82, respectively. In other embodiments, the antibody comprises (i) a VH comprising a VH CDR1, a VH CDR2, and a VH CDR3 having an amino acid sequence of SEQ ID NOs: 83, 84 and 85, respectively; and (ii) a VL comprising a VL CDR1, a VL CDR2, and a VL CDR3 having an amino acid sequence of SEQ ID NOs: 86, 87 and 88, respectively. In some embodiments, the antibody comprises (i) a VH comprising a VH CDR1, a VH CDR2, and a VH CDR3 having an amino acid sequence of SEQ ID NOs: 89, 90 and 91, respectively; and (ii) a VL comprising a VL CDR1, a VL CDR2, and a VL CDR3 having an amino acid sequence of SEQ ID NOs: 92, 93 and 94, respectively. In one embodiment, the antibody comprises a VH having an amino acid sequence having at least 95% identity to an amino acid sequence of SEQ ID NO:95. In one embodiment, the antibody comprises a VL having an amino acid sequence having at least 95% identity to an amino acid sequence of SEQ ID NO:96. In another embodiment, the antibody comprises (i) a VH having an amino acid sequence having at least 95% identity to an amino acid sequence of SEQ ID NO:95; and (ii) a VL having an amino acid sequence having at least 95% identity to an amino acid sequence of SEQ ID NO:96. In one embodiment, the antibody comprises a VH having an amino acid sequence SEQ ID NO:95. In another embodiment, the antibody comprises a VL having an amino acid sequence SEQ ID NO:96. In one embodiment, the antibody comprises (i) a VH having an amino acid sequence SEQ ID NO:95; and (ii) a VL having an amino acid sequence SEQ ID NO:96. In one embodiment, the antibody comprises a heavy chain having an amino acid sequence having at least 95% identity to an amino acid sequence of SEQ ID NO:97. In another embodiment, the antibody comprises a light chain having an amino acid sequence having at least 95% identity to an amino acid sequence of SEQ ID NO:98. In one embodiment, the antibody comprises (i) a heavy chain having an amino acid sequence having at least 95% identity to an amino acid sequence of SEQ ID NO:97; and (ii) a light chain having an amino acid sequence having at least 95% identity to an amino acid sequence of SEQ ID NO:98. In one embodiment, the antibody comprises a heavy chain having an amino acid sequence SEQ ID NO:97. In another embodiment, the antibody comprises a light chain having an amino acid sequence SEQ ID NO:98. In one embodiment, the antibody comprises (i) a heavy chain having an amino acid sequence SEQ ID NO:97; and (ii) a light chain having an amino acid sequence SEQ ID NO:98.

In some embodiments, the antibody that binds to Sdc2 is antibody clone TP-43329F. In certain embodiments, the antibody comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 129; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 130. In some embodiments, the antibody comprises (i) a VH comprising a VH CDR1, a VH CDR2, and a VH CDR3 having an amino acid sequence of SEQ ID NOs: 99, 100 and 101, respectively; and (ii) a VL comprising a VL CDR1, a VL CDR2, and a VL CDR3 having an amino acid sequence of SEQ ID NOs: 102,103 and 104, respectively. In other embodiments, the antibody comprises (i) a VH comprising a VH CDR1, a VH CDR2, and a VH CDR3 having an amino acid sequence of SEQ ID NOs: 105, 106 and 107, respectively; and (ii) a VL comprising a VL CDR1, a VL CDR2, and a VL CDR3 having an amino acid sequence of SEQ ID NOs: 108, 109 and 110, respectively. In some embodiments, the antibody comprises (i) a VH comprising a VH CDR1, a VH CDR2, and a VH CDR3 having an amino acid sequence of SEQ ID NOs: 111, 112 and 113, respectively; and (ii) a VL comprising a VL CDR1, a VL CDR2, and a VL CDR3 having an amino acid sequence of SEQ ID NOs: 114, 115 and 116, respectively. In other embodiments, the antibody comprises (i) a VH comprising a VH CDR1, a VH CDR2, and a VH CDR3 having an amino acid sequence of SEQ ID NOs: 117, 118 and 119, respectively; and (ii) a VL comprising a VL CDR1, a VL CDR2, and a VL CDR3 having an amino acid sequence of SEQ ID NOs: 120, 121 and 122, respectively. In some embodiments, the antibody comprises (i) a VH comprising a VH CDR1, a VH CDR2, and a VH CDR3 having an amino acid sequence of SEQ ID NOs: 123, 124 and 125, respectively; and (ii) a VL comprising a VL CDR1, a VL CDR2, and a VL CDR3 having an amino acid sequence of SEQ ID NOs: 126, 127 and 128, respectively. In one embodiment, the antibody comprises a VH having an amino acid sequence having at least 95% identity to an amino acid sequence of SEQ ID NO:129. In one embodiment, the antibody comprises a VL having an amino acid sequence having at least 95% identity to an amino acid sequence of SEQ ID NO: 130. In another embodiment, the antibody comprises (i) a VH having an amino acid sequence having at least 95% identity to an amino acid sequence of SEQ ID NO: 129; and (ii) a VL having an amino acid sequence having at least 95% identity to an amino acid sequence of SEQ ID NO: 130. In one embodiment, the antibody comprises a VH having an amino acid sequence SEQ ID NO:129. In another embodiment, the antibody comprises a VL having an amino acid sequence SEQ ID NO:130. In one embodiment, the antibody comprises (i) a VH having an amino acid sequence SEQ ID NO:129; and (ii) a VL having an amino acid sequence SEQ ID NO:130. In one embodiment, the antibody comprises a heavy chain having an amino acid sequence having at least 95% identity to an amino acid sequence of SEQ ID NO: 131. In another embodiment, the antibody comprises a light chain having an amino acid sequence having at least 95% identity to an amino acid sequence of SEQ ID NO: 132. In one embodiment, the antibody comprises (i) a heavy chain having an amino acid sequence having at least 95% identity to an amino acid sequence of SEQ ID NO: 131; and (ii) a light chain having an amino acid sequence having at least 95% identity to an amino acid sequence of SEQ ID NO: 132. In one embodiment, the antibody comprises a heavy chain having an amino acid sequence SEQ ID NO: 131. In another embodiment, the antibody comprises a light chain having an amino acid sequence SEQ ID NO: 132. In one embodiment, the antibody comprises (i) a heavy chain having an amino acid sequence SEQ ID NO: 131; and (ii) a light chain having an amino acid sequence SEQ ID NO: 132.

In some embodiments, the antibody that binds to Sdc2 is antibody clone 8-G17A. In certain embodiments, the antibody comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 163; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 164. In some embodiments, the antibody comprises (i) a VH comprising a VH CDR1, a VH CDR2, and a VH CDR3 having an amino acid sequence of SEQ ID NOs: 133, 134 and 135, respectively; and (ii) a VL comprising a VL CDR1, a VL CDR2, and a VL CDR3 having an amino acid sequence of SEQ ID NOs: 136, 137 and 138, respectively. In other embodiments, the antibody comprises (i) a VH comprising a VH CDR1, a VH CDR2, and a VH CDR3 having an amino acid sequence of SEQ ID NOs: 139, 140 and 141, respectively; and (ii) a VL comprising a VL CDR1, a VL CDR2, and a VL CDR3 having an amino acid sequence of SEQ ID NOs: 142, 143 and 144, respectively. In some embodiments, the antibody comprises (i) a VH comprising a VH CDR1, a VH CDR2, and a VH CDR3 having an amino acid sequence of SEQ ID NOs: 145, 146 and 147, respectively; and (ii) a VL comprising a VL CDR1, a VL CDR2, and a VL CDR3 having an amino acid sequence of SEQ ID NOs: 148, 149 and 150, respectively. In other embodiments, the antibody comprises (i) a VH comprising a VH CDR1, a VH CDR2, and a VH CDR3 having an amino acid sequence of SEQ ID NOs: 151, 152 and 153, respectively; and (ii) a VL comprising a VL CDR1, a VL CDR2, and a VL CDR3 having an amino acid sequence of SEQ ID NOs: 154, 155 and 156, respectively. In some embodiments, the antibody comprises (i) a VH comprising a VH CDR1, a VH CDR2, and a VH CDR3 having an amino acid sequence of SEQ ID NOs: 157, 158 and 159, respectively; and (ii) a VL comprising a VL CDR1, a VL CDR2, and a VL CDR3 having an amino acid sequence of SEQ ID NOs: 160, 161 and 162, respectively. In one embodiment, the antibody comprises a VH having an amino acid sequence having at least 95% identity to an amino acid sequence of SEQ ID NO: 163. In another embodiment, the antibody comprises a VL having an amino acid sequence having at least 95% identity to an amino acid sequence of SEQ ID NO: 164. In one embodiment, the antibody comprises (i) a VH having an amino acid sequence having at least 95% identity to an amino acid sequence of SEQ ID NO: 163; and (ii) a VL having an amino acid sequence having at least 95% identity to an amino acid sequence of SEQ ID NO: 164. In one embodiment, the antibody comprises a VH having an amino acid sequence SEQ ID NO:163. In another embodiment, the antibody comprises a VL having an amino acid sequence SEQ ID NO:164. In one embodiment, the antibody comprises (i) a VH having an amino acid sequence SEQ ID NO: 163; and (ii) a VL having an amino acid sequence SEQ ID NO:164. In one embodiment, the antibody comprises a heavy chain having an amino acid sequence having at least 95% identity to an amino acid sequence of SEQ ID NO: 165. In another embodiment, the antibody comprises a light chain having an amino acid sequence having at least 95% identity to an amino acid sequence of SEQ ID NO: 166. In one embodiment, the antibody comprises (i) a heavy chain having an amino acid sequence having at least 95% identity to an amino acid sequence of SEQ ID NO: 165; and (ii) a light chain having an amino acid sequence having at least 95% identity to an amino acid sequence of SEQ ID NO: 166. In one embodiment, the antibody comprises a heavy chain having an amino acid sequence SEQ ID NO: 165. In another embodiment, the antibody comprises a light chain having an amino acid sequence SEQ ID NO: 166. In one embodiment, the antibody comprises (i) a heavy chain having an amino acid sequence SEQ ID NO:165; and (ii) a light chain having an amino acid sequence SEQ ID NO:166.

In some embodiments, the antibody that binds to Sdc2 is antibody clone 6-N03-A. In certain embodiments, the antibody comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 197; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 198. In some embodiments, the antibody comprises (i) a VH comprising a VH CDR1, a VH CDR2, and a VH CDR3 having an amino acid sequence of SEQ ID NOs: 167, 168 and 169, respectively; and (ii) a VL comprising a VL CDR1, a VL CDR2, and a VL CDR3 having an amino acid sequence of SEQ ID NOs: 170, 171 and 172, respectively. In other embodiments, the antibody comprises (i) a VH comprising a VH CDR1, a VH CDR2, and a VH CDR3 having an amino acid sequence of SEQ ID NOs: 173, 174 and 175, respectively; and (ii) a VL comprising a VL CDR1, a VL CDR2, and a VL CDR3 having an amino acid sequence of SEQ ID NOs: 176, 177 and 178, respectively. In some embodiments, the antibody comprises (i) a VH comprising a VH CDR1, a VH CDR2, and a VH CDR3 having an amino acid sequence of SEQ ID NOs: 179, 180 and 181, respectively; and (ii) a VL comprising a VL CDR1, a VL CDR2, and a VL CDR3 having an amino acid sequence of SEQ ID NOs: 182, 183 and 184, respectively. In other embodiments, the antibody comprises (i) a VH comprising a VH CDR1, a VH CDR2, and a VH CDR3 having an amino acid sequence of SEQ ID NOs: 185, 186 and 187, respectively; and (ii) a VL comprising a VL CDR1, a VL CDR2, and a VL CDR3 having an amino acid sequence of SEQ ID NOs: 188, 189 and 190, respectively. In some embodiments, the antibody comprises (i) a VH comprising a VH CDR1, a VH CDR2, and a VH CDR3 having an amino acid sequence of SEQ ID NOs: 191, 192 and 193, respectively; and (ii) a VL comprising a VL CDR1, a VL CDR2, and a VL CDR3 having an amino acid sequence of SEQ ID NOs: 194, 195 and 196, respectively. In one embodiment, the antibody comprises a VH having an amino acid sequence having at least 95% identity to an amino acid sequence of SEQ ID NO: 197. In another embodiment, the antibody comprises a VL having an amino acid sequence having at least 95% identity to an amino acid sequence of SEQ ID NO: 198. In one embodiment, the antibody comprises (i) a VH having an amino acid sequence having at least 95% identity to an amino acid sequence of SEQ ID NO: 197; and (ii) a VL having an amino acid sequence having at least 95% identity to an amino acid sequence of SEQ ID NO:198. In one embodiment, the antibody comprises a VH having an amino acid sequence SEQ ID NO:197. In another embodiment, the antibody comprises a VL having an amino acid sequence SEQ ID NO:198. In one embodiment, the antibody comprises (i) a VH having an amino acid sequence SEQ ID NO: 197; and (ii) a VL having an amino acid sequence SEQ ID NO: 198. In one embodiment, the antibody comprises a heavy chain having an amino acid sequence having at least 95% identity to an amino acid sequence of SEQ ID NO: 199. In another embodiment, the antibody comprises a light chain having an amino acid sequence having at least 95% identity to an amino acid sequence of SEQ ID NO: 200. In one embodiment, the antibody comprises (i) a heavy chain having an amino acid sequence having at least 95% identity to an amino acid sequence of SEQ ID NO: 199; and (ii) a light chain having an amino acid sequence having at least 95% identity to an amino acid sequence of SEQ ID NO:200. In one embodiment, the antibody comprises a heavy chain having an amino acid sequence SEQ ID NO:199. In another embodiment, the antibody comprises a light chain having an amino acid sequence SEQ ID NO: 200. In one embodiment, the antibody comprises (i) a heavy chain having an amino acid sequence SEQ ID NO: 199; and (ii) a light chain having an amino acid sequence SEQ ID NO:200.

In some embodiments, the antibody that binds to Sdc2 is antibody clone R3-P3-C11. In certain embodiments, the antibody comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:231; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:232. In some embodiments, the antibody comprises (i) a VH comprising a VH CDR1, a VH CDR2, and a VH CDR3 having an amino acid sequence of SEQ ID NOs: 201, 202 and 203, respectively; and (ii) a VL comprising a VL CDR1, a VL CDR2, and a VL CDR3 having an amino acid sequence of SEQ ID NOs: 204, 205 and 206, respectively. In other embodiments, the antibody comprises (i) a VH comprising a VH CDR1, a VH CDR2, and a VH CDR3 having an amino acid sequence of SEQ ID NOs: 207, 208 and 209, respectively; and (ii) a VL comprising a VL CDR1, a VL CDR2, and a VL CDR3 having an amino acid sequence of SEQ ID NOs: 210, 211 and 212, respectively. In some embodiments, the antibody comprises (i) a VH comprising a VH CDR1, a VH CDR2, and a VH CDR3 having an amino acid sequence of SEQ ID NOs: 213, 214 and 215, respectively; and (ii) a VL comprising a VL CDR1, a VL CDR2, and a VL CDR3 having an amino acid sequence of SEQ ID NOs: 216, 217 and 218, respectively. In other embodiments, the antibody comprises (i) a VH comprising a VH CDR1, a VH CDR2, and a VH CDR3 having an amino acid sequence of SEQ ID NOs: 219, 220 and 221, respectively; and (ii) a VL comprising a VL CDR1, a VL CDR2, and a VL CDR3 having an amino acid sequence of SEQ ID NOs: 222, 223 and 224, respectively. In some embodiments, the antibody comprises (i) a VH comprising a VH CDR1, a VH CDR2, and a VH CDR3 having an amino acid sequence of SEQ ID NOs: 225, 226 and 227, respectively; and (ii) a VL comprising a VL CDR1, a VL CDR2, and a VL CDR3 having an amino acid sequence of SEQ ID NOs: 228, 229 and 230, respectively. In one embodiment, the antibody comprises a VH having an amino acid sequence having at least 95% identity to an amino acid sequence of SEQ ID NO:231. In another embodiment, the antibody comprises a VL having an amino acid sequence having at least 95% identity to an amino acid sequence of SEQ ID NO:232. In one embodiment, the antibody comprises (i) a VH having an amino acid sequence having at least 95% identity to an amino acid sequence of SEQ ID NO: 231; and (ii) a VL having an amino acid sequence having at least 95% identity to an amino acid sequence of SEQ ID NO:232. In one embodiment, the antibody comprises a VH having an amino acid sequence SEQ ID NO:231. In another embodiment, the antibody comprises a VL having an amino acid sequence SEQ ID NO:232. In one embodiment, the antibody comprises (i) a VH having an amino acid sequence SEQ ID NO:231; and (ii) a VL having an amino acid sequence SEQ ID NO:232. In one embodiment, the antibody comprises a heavy chain having an amino acid sequence having at least 95% identity to an amino acid sequence of SEQ ID NO:233. In another embodiment, the antibody comprises a light chain having an amino acid sequence having at least 95% identity to an amino acid sequence of SEQ ID NO: 234. In one embodiment, the antibody comprises (i) a heavy chain having an amino acid sequence having at least 95% identity to an amino acid sequence of SEQ ID NO:233; and (ii) a light chain having an amino acid sequence having at least 95% identity to an amino acid sequence of SEQ ID NO:234. In one embodiment, the antibody comprises a heavy chain having an amino acid sequence SEQ ID NO:233. In another embodiment, the antibody comprises a light chain having an amino acid sequence SEQ ID NO: 234. In one embodiment, the antibody comprises (i) a heavy chain having an amino acid sequence SEQ ID NO:233; and (ii) a light chain having an amino acid sequence SEQ ID NO:234.

In some embodiments, the antibody that binds to Sdc2 is antibody clone R4M-P3-E06. In certain embodiments, the antibody comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:265; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:266. In some embodiments, the antibody comprises (i) a VH comprising a VH CDR1, a VH CDR2, and a VH CDR3 having an amino acid sequence of SEQ ID NOs: 235, 236 and 237, respectively; and (ii) a VL comprising a VL CDR1, a VL CDR2, and a VL CDR3 having an amino acid sequence of SEQ ID NOs: 238, 239 and 240, respectively. In other embodiments, the antibody comprises (i) a VH comprising a VH CDR1, a VH CDR2, and a VH CDR3 having an amino acid sequence of SEQ ID NOs: 241, 242 and 243, respectively; and (ii) a VL comprising a VL CDR1, a VL CDR2, and a VL CDR3 having an amino acid sequence of SEQ ID NOs: 244, 245 and 246, respectively. In some embodiments, the antibody comprises (i) a VH comprising a VH CDR1, a VH CDR2, and a VH CDR3 having an amino acid sequence of SEQ ID NOs: 247, 248 and 249, respectively; and (ii) a VL comprising a VL CDR1, a VL CDR2, and a VL CDR3 having an amino acid sequence of SEQ ID NOs: 250, 251 and 252, respectively. In other embodiments, the antibody comprises (i) a VH comprising a VH CDR1, a VH CDR2, and a VH CDR3 having an amino acid sequence of SEQ ID NOs: 253, 254 and 255, respectively; and (ii) a VL comprising a VL CDR1, a VL CDR2, and a VL CDR3 having an amino acid sequence of SEQ ID NOs: 256, 257 and 258, respectively. In some embodiments, the antibody comprises (i) a VH comprising a VH CDR1, a VH CDR2, and a VH CDR3 having an amino acid sequence of SEQ ID NOs: 259, 260 and 261, respectively; and (ii) a VL comprising a VL CDR1, a VL CDR2, and a VL CDR3 having an amino acid sequence of SEQ ID NOs: 262, 263 and 264, respectively. In one embodiment, the antibody comprises a VH having an amino acid sequence having at least 95% identity to an amino acid sequence of SEQ ID NO:265. In another embodiment, the antibody comprises a VL having an amino acid sequence having at least 95% identity to an amino acid sequence of SEQ ID NO 266. In one embodiment, the antibody comprises (i) a VH having an amino acid sequence having at least 95% identity to an amino acid sequence of SEQ ID NO: 265; and (ii) a VL having an amino acid sequence having at least 95% identity to an amino acid sequence of SEQ ID NO 266. In one embodiment, the antibody comprises a VH having an amino acid sequence SEQ ID NO:265. In another embodiment, the antibody comprises a VL having an amino acid sequence SEQ ID NO:266. In one embodiment, the antibody comprises (i) a VH having an amino acid sequence SEQ ID NO:265; and (ii) a VL having an amino acid sequence SEQ ID NO:266. In one embodiment, the antibody comprises a heavy chain having an amino acid sequence having at least 95% identity to an amino acid sequence of SEQ ID NO:267. In another embodiment, the antibody comprises a light chain having an amino acid sequence having at least 95% identity to an amino acid sequence of SEQ ID NO: 268. In one embodiment, the antibody comprises (i) a heavy chain having an amino acid sequence having at least 95% identity to an amino acid sequence of SEQ ID NO:267; and (ii) a light chain having an amino acid sequence having at least 95% identity to an amino acid sequence of SEQ ID NO:268. In one embodiment, the antibody comprises a heavy chain having an amino acid sequence SEQ ID NO:267. In another embodiment, the antibody comprises a light chain having an amino acid sequence SEQ ID NO: 268. In one embodiment, the antibody comprises (i) a heavy chain having an amino acid sequence SEQ ID NO:267; and (ii) a light chain having an amino acid sequence SEQ ID NO:268.

In some embodiments, the antibody that binds to Sdc2 is antibody clone R3-P3-E09. In certain embodiments, the antibody comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:299; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:300. In some embodiments, the antibody comprises (i) a VH comprising a VH CDR1, a VH CDR2, and a VH CDR3 having an amino acid sequence of SEQ ID NOs: 269, 270 and 271, respectively; and (ii) a VL comprising a VL CDR1, a VL CDR2, and a VL CDR3 having an amino acid sequence of SEQ ID NOs: 272, 273 and 274, respectively. In other embodiments, the antibody comprises (i) a VH comprising a VH CDR1, a VH CDR2, and a VH CDR3 having an amino acid sequence of SEQ ID NOs: 275, 276 and 277, respectively; and (ii) a VL comprising a VL CDR1, a VL CDR2, and a VL CDR3 having an amino acid sequence of SEQ ID NOs: 278, 279 and 280, respectively. In some embodiments, the antibody comprises (i) a VH comprising a VH CDR1, a VH CDR2, and a VH CDR3 having an amino acid sequence of SEQ ID NOs: 281, 282 and 283, respectively; and (ii) a VL comprising a VL CDR1, a VL CDR2, and a VL CDR3 having an amino acid sequence of SEQ ID NOs: 284, 285 and 286, respectively. In other embodiments, the antibody comprises (i) a VH comprising a VH CDR1, a VH CDR2, and a VH CDR3 having an amino acid sequence of SEQ ID NOs: 287, 288 and 289, respectively; and (ii) a VL comprising a VL CDR1, a VL CDR2, and a VL CDR3 having an amino acid sequence of SEQ ID NOs: 290, 292 and 292, respectively. In some embodiments, the antibody comprises (i) a VH comprising a VH CDR1, a VH CDR2, and a VH CDR3 having an amino acid sequence of SEQ ID NOs: 293, 294 and 295, respectively; and (ii) a VL comprising a VL CDR1, a VL CDR2, and a VL CDR3 having an amino acid sequence of SEQ ID NOs: 296, 297 and 298, respectively. In one embodiment, the antibody comprises a VH having an amino acid sequence having at least 95% identity to an amino acid sequence of SEQ ID NO:299. In another embodiment, the antibody comprises a VL having an amino acid sequence having at least 95% identity to an amino acid sequence of SEQ ID NO:300. In one embodiment, the antibody comprises (i) a VH having an amino acid sequence having at least 95% identity to an amino acid sequence of SEQ ID NO: 299; and (ii) a VL having an amino acid sequence having at least 95% identity to an amino acid sequence of SEQ ID NO:300. In one embodiment, the antibody comprises a VH having an amino acid sequence SEQ ID NO:299. In another embodiment, the antibody comprises a VL having an amino acid sequence SEQ ID NO:300. In one embodiment, the antibody comprises (i) a VH having an amino acid sequence SEQ ID NO:299; and (ii) a VL having an amino acid sequence SEQ ID NO:300. In one embodiment, the antibody comprises a heavy chain having an amino acid sequence having at least 95% identity to an amino acid sequence of SEQ ID NO:301. In another embodiment, the antibody comprises a light chain having an amino acid sequence having at least 95% identity to an amino acid sequence of SEQ ID NO: 302. In one embodiment, the antibody comprises (i) a heavy chain having an amino acid sequence having at least 95% identity to an amino acid sequence of SEQ ID NO:301; and (ii) a light chain having an amino acid sequence having at least 95% identity to an amino acid sequence of SEQ ID NO:302. In one embodiment, the antibody comprises a heavy chain having an amino acid sequence SEQ ID NO:301. In another embodiment, the antibody comprises a light chain having an amino acid sequence SEQ ID NO: 302. In one embodiment, the antibody comprises (i) a heavy chain having an amino acid sequence SEQ ID NO:301; and (ii) a light chain having an amino acid sequence SEQ ID NO:302.

In some embodiments, the antibody that binds to Sdc2 is antibody clone R3-P1-C02. In certain embodiments, the antibody comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:333; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:334. In some embodiments, the antibody comprises (i) a VH comprising a VH CDR1, a VH CDR2, and a VH CDR3 having an amino acid sequence of SEQ ID NOs: 303, 304 and 305, respectively; and (ii) a VL comprising a VL CDR1, a VL CDR2, and a VL CDR3 having an amino acid sequence of SEQ ID NOs: 306, 307 and 308, respectively. In other embodiments, the antibody comprises (i) a VH comprising a VH CDR1, a VH CDR2, and a VH CDR3 having an amino acid sequence of SEQ ID NOs: 309, 310 and 311, respectively; and (ii) a VL comprising a VL CDR1, a VL CDR2, and a VL CDR3 having an amino acid sequence of SEQ ID NOs: 312, 313 and 314, respectively. In some embodiments, the antibody comprises (i) a VH comprising a VH CDR1, a VH CDR2, and a VH CDR3 having an amino acid sequence of SEQ ID NOs: 315, 316 and 317, respectively; and (ii) a VL comprising a VL CDR1, a VL CDR2, and a VL CDR3 having an amino acid sequence of SEQ ID NOs: 318, 319 and 320, respectively. In other embodiments, the antibody comprises (i) a VH comprising a VH CDR1, a VH CDR2, and a VH CDR3 having an amino acid sequence of SEQ ID NOs: 321, 322 and 323, respectively; and (ii) a VL comprising a VL CDR1, a VL CDR2, and a VL CDR3 having an amino acid sequence of SEQ ID NOs: 324, 325 and 326, respectively. In some embodiments, the antibody comprises (i) a VH comprising a VH CDR1, a VH CDR2, and a VH CDR3 having an amino acid sequence of SEQ ID NOs: 327, 328 and 329, respectively; and (ii) a VL comprising a VL CDR1, a VL CDR2, and a VL CDR3 having an amino acid sequence of SEQ ID NOs: 330, 331 and 332, respectively. In one embodiment, the antibody comprises a VH having an amino acid sequence having at least 95% identity to an amino acid sequence of SEQ ID NO:333. In another embodiment, the antibody comprises a VL having an amino acid sequence having at least 95% identity to an amino acid sequence of SEQ ID NO:334. In one embodiment, the antibody comprises (i) a VH having an amino acid sequence having at least 95% identity to an amino acid sequence of SEQ ID NO: 333; and (ii) a VL having an amino acid sequence having at least 95% identity to an amino acid sequence of SEQ ID NO:334. In one embodiment, the antibody comprises a VH having an amino acid sequence SEQ ID NO:333. In another embodiment, the antibody comprises a VL having an amino acid sequence SEQ ID NO:334. In one embodiment, the antibody comprises (i) a VH having an amino acid sequence SEQ ID NO:333; and (ii) a VL having an amino acid sequence SEQ ID NO:334. In one embodiment, the antibody comprises a heavy chain having an amino acid sequence having at least 95% identity to an amino acid sequence of SEQ ID NO:335. In another embodiment, the antibody comprises a light chain having an amino acid sequence having at least 95% identity to an amino acid sequence of SEQ ID NO: 336. In one embodiment, the antibody comprises (i) a heavy chain having an amino acid sequence having at least 95% identity to an amino acid sequence of SEQ ID NO:335; and (ii) a light chain having an amino acid sequence having at least 95% identity to an amino acid sequence of SEQ ID NO:336. In one embodiment, the antibody comprises a heavy chain having an amino acid sequence SEQ ID NO:335. In another embodiment, the antibody comprises a light chain having an amino acid sequence SEQ ID NO: 336. In one embodiment, the antibody comprises (i) a heavy chain having an amino acid sequence SEQ ID NO:335; and (ii) a light chain having an amino acid sequence SEQ ID NO:336.

In some embodiments, the antibody that binds to Sdc2 is antibody clone R3-P3-A12. In certain embodiments, the antibody comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:367; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:368. In some embodiments, the antibody comprises (i) a VH comprising a VH CDR1, a VH CDR2, and a VH CDR3 having an amino acid sequence of SEQ ID NOs: 337, 338 and 339, respectively; and (ii) a VL comprising a VL CDR1, a VL CDR2, and a VL CDR3 having an amino acid sequence of SEQ ID NOs: 340, 341 and 342, respectively. In other embodiments, the antibody comprises (i) a VH comprising a VH CDR1, a VH CDR2, and a VH CDR3 having an amino acid sequence of SEQ ID NOs: 343, 344 and 345, respectively; and (ii) a VL comprising a VL CDR1, a VL CDR2, and a VL CDR3 having an amino acid sequence of SEQ ID NOs: 346, 347 and 348, respectively. In some embodiments, the antibody comprises (i) a VH comprising a VH CDR1, a VH CDR2, and a VH CDR3 having an amino acid sequence of SEQ ID NOs: 349, 350 and 351, respectively; and (ii) a VL comprising a VL CDR1, a VL CDR2, and a VL CDR3 having an amino acid sequence of SEQ ID NOs: 352, 353 and 354, respectively. In other embodiments, the antibody comprises (i) a VH comprising a VH CDR1, a VH CDR2, and a VH CDR3 having an amino acid sequence of SEQ ID NOs: 355, 356 and 357, respectively; and (ii) a VL comprising a VL CDR1, a VL CDR2, and a VL CDR3 having an amino acid sequence of SEQ ID NOs: 358, 359 and 360, respectively. In some embodiments, the antibody comprises (i) a VH comprising a VH CDR1, a VH CDR2, and a VH CDR3 having an amino acid sequence of SEQ ID NOs: 361, 362 and 363, respectively; and (ii) a VL comprising a VL CDR1, a VL CDR2, and a VL CDR3 having an amino acid sequence of SEQ ID NOs: 364, 365 and 366, respectively. In one embodiment, the antibody comprises a VH having an amino acid sequence having at least 95% identity to an amino acid sequence of SEQ ID NO:367. In another embodiment, the antibody comprises a VL having an amino acid sequence having at least 95% identity to an amino acid sequence of SEQ ID NO:368. In one embodiment, the antibody comprises (i) a VH having an amino acid sequence having at least 95% identity to an amino acid sequence of SEQ ID NO: 367; and (ii) a VL having an amino acid sequence having at least 95% identity to an amino acid sequence of SEQ ID NO:368. In one embodiment, the antibody comprises a VH having an amino acid sequence SEQ ID NO:367. In another embodiment, the antibody comprises a VL having an amino acid sequence SEQ ID NO:368. In one embodiment, the antibody comprises (i) a VH having an amino acid sequence SEQ ID NO:367; and (ii) a VL having an amino acid sequence SEQ ID NO:368. In one embodiment, the antibody comprises a heavy chain having an amino acid sequence having at least 95% identity to an amino acid sequence of SEQ ID NO:369. In another embodiment, the antibody comprises a light chain having an amino acid sequence having at least 95% identity to an amino acid sequence of SEQ ID NO: 370. In one embodiment, the antibody comprises (i) a heavy chain having an amino acid sequence having at least 95% identity to an amino acid sequence of SEQ ID NO:369; and (ii) a light chain having an amino acid sequence having at least 95% identity to an amino acid sequence of SEQ ID NO:370. In one embodiment, the antibody comprises a heavy chain having an amino acid sequence SEQ ID NO:369. In another embodiment, the antibody comprises a light chain having an amino acid sequence SEQ ID NO: 370. In one embodiment, the antibody comprises (i) a heavy chain having an amino acid sequence SEQ ID NO:369; and (ii) a light chain having an amino acid sequence SEQ ID NO:370.

In some embodiments, the antibody that binds to Sdc2 is antibody clone R4M-P3-A12. In certain embodiments, the antibody comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:401; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:402. In some embodiments, the antibody comprises (i) a VH comprising a VH CDR1, a VH CDR2, and a VH CDR3 having an amino acid sequence of SEQ ID NOs: 371, 372 and 373, respectively; and (ii) a VL comprising a VL CDR1, a VL CDR2, and a VL CDR3 having an amino acid sequence of SEQ ID NOs: 374, 375 and 376, respectively. In other embodiments, the antibody comprises (i) a VH comprising a VH CDR1, a VH CDR2, and a VH CDR3 having an amino acid sequence of SEQ ID NOs: 377, 378 and 379, respectively; and (ii) a VL comprising a VL CDR1, a VL CDR2, and a VL CDR3 having an amino acid sequence of SEQ ID NOs: 380, 381 and 382, respectively. In some embodiments, the antibody comprises (i) a VH comprising a VH CDR1, a VH CDR2, and a VH CDR3 having an amino acid sequence of SEQ ID NOs: 383, 384 and 385, respectively; and (ii) a VL comprising a VL CDR1, a VL CDR2, and a VL CDR3 having an amino acid sequence of SEQ ID NOs: 386, 387 and 388, respectively. In other embodiments, the antibody comprises (i) a VH comprising a VH CDR1, a VH CDR2, and a VH CDR3 having an amino acid sequence of SEQ ID NOs: 389, 390 and 391, respectively; and (ii) a VL comprising a VL CDR1, a VL CDR2, and a VL CDR3 having an amino acid sequence of SEQ ID NOs: 392, 393 and 394, respectively. In some embodiments, the antibody comprises (i) a VH comprising a VH CDR1, a VH CDR2, and a VH CDR3 having an amino acid sequence of SEQ ID NOs: 395, 396 and 397, respectively; and (ii) a VL comprising a VL CDR1, a VL CDR2, and a VL CDR3 having an amino acid sequence of SEQ ID NOs: 398, 399 and 400, respectively. In one embodiment, the antibody comprises a VH having an amino acid sequence having at least 95% identity to an amino acid sequence of SEQ ID NO:401. In another embodiment, the antibody comprises a VL having an amino acid sequence having at least 95% identity to an amino acid sequence of SEQ ID NO:402. In one embodiment, the antibody comprises (i) a VH having an amino acid sequence having at least 95% identity to an amino acid sequence of SEQ ID NO: 401; and (ii) a VL having an amino acid sequence having at least 95% identity to an amino acid sequence of SEQ ID NO:402. In one embodiment, the antibody comprises a VH having an amino acid sequence SEQ ID NO:401. In another embodiment, the antibody comprises a VL having an amino acid sequence SEQ ID NO:402. In one embodiment, the antibody comprises (i) a VH having an amino acid sequence SEQ ID NO:401; and (ii) a VL having an amino acid sequence SEQ ID NO:402. In one embodiment, the antibody comprises a heavy chain having an amino acid sequence having at least 95% identity to an amino acid sequence of SEQ ID NO:403. In another embodiment, the antibody comprises a light chain having an amino acid sequence having at least 95% identity to an amino acid sequence of SEQ ID NO: 404. In one embodiment, the antibody comprises (i) a heavy chain having an amino acid sequence having at least 95% identity to an amino acid sequence of SEQ ID NO:403; and (ii) a light chain having an amino acid sequence having at least 95% identity to an amino acid sequence of SEQ ID NO:404. In one embodiment, the antibody comprises a heavy chain having an amino acid sequence SEQ ID NO:403. In another embodiment, the antibody comprises a light chain having an amino acid sequence SEQ ID NO: 404. In one embodiment, the antibody comprises (i) a heavy chain having an amino acid sequence SEQ ID NO:403; and (ii) a light chain having an amino acid sequence SEQ ID NO:404.

In some embodiments, the antibody that binds to Sdc2 is antibody clone R4M-P1-A10. In certain embodiments, the antibody comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:435; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:436. In some embodiments, the antibody comprises (i) a VH comprising a VH CDR1, a VH CDR2, and a VH CDR3 having an amino acid sequence of SEQ ID NOs: 405, 406 and 407, respectively; and (ii) a VL comprising a VL CDR1, a VL CDR2, and a VL CDR3 having an amino acid sequence of SEQ ID NOs: 408, 409 and 410, respectively. In other embodiments, the antibody comprises (i) a VH comprising a VH CDR1, a VH CDR2, and a VH CDR3 having an amino acid sequence of SEQ ID NOs: 411, 412 and 413, respectively; and (ii) a VL comprising a VL CDR1, a VL CDR2, and a VL CDR3 having an amino acid sequence of SEQ ID NOs: 414, 415 and 416, respectively. In some embodiments, the antibody comprises (i) a VH comprising a VH CDR1, a VH CDR2, and a VH CDR3 having an amino acid sequence of SEQ ID NOs: 417, 418 and 419, respectively; and (ii) a VL comprising a VL CDR1, a VL CDR2, and a VL CDR3 having an amino acid sequence of SEQ ID NOs: 420, 421 and 422, respectively. In other embodiments, the antibody comprises (i) a VH comprising a VH CDR1, a VH CDR2, and a VH CDR3 having an amino acid sequence of SEQ ID NOs: 423, 424 and 425, respectively; and (ii) a VL comprising a VL CDR1, a VL CDR2, and a VL CDR3 having an amino acid sequence of SEQ ID NOs: 426, 427 and 428, respectively. In some embodiments, the antibody comprises (i) a VH comprising a VH CDR1, a VH CDR2, and a VH CDR3 having an amino acid sequence of SEQ ID NOs: 429, 430 and 431, respectively; and (ii) a VL comprising a VL CDR1, a VL CDR2, and a VL CDR3 having an amino acid sequence of SEQ ID NOs: 432, 433 and 434, respectively. In one embodiment, the antibody comprises a VH having an amino acid sequence having at least 95% identity to an amino acid sequence of SEQ ID NO:435. In another embodiment, the antibody comprises a VL having an amino acid sequence having at least 95% identity to an amino acid sequence of SEQ ID NO:436. In one embodiment, the antibody comprises (i) a VH having an amino acid sequence having at least 95% identity to an amino acid sequence of SEQ ID NO: 435; and (ii) a VL having an amino acid sequence having at least 95% identity to an amino acid sequence of SEQ ID NO:436. In one embodiment, the antibody comprises a VH having an amino acid sequence SEQ ID NO:435. In another embodiment, the antibody comprises a VL having an amino acid sequence SEQ ID NO:436. In one embodiment, the antibody comprises (i) a VH having an amino acid sequence SEQ ID NO:435; and (ii) a VL having an amino acid sequence SEQ ID NO:436. In one embodiment, the antibody comprises a heavy chain having an amino acid sequence having at least 95% identity to an amino acid sequence of SEQ ID NO:437. In another embodiment, the antibody comprises a light chain having an amino acid sequence having at least 95% identity to an amino acid sequence of SEQ ID NO: 438. In one embodiment, the antibody comprises (i) a heavy chain having an amino acid sequence having at least 95% identity to an amino acid sequence of SEQ ID NO:437; and (ii) a light chain having an amino acid sequence having at least 95% identity to an amino acid sequence of SEQ ID NO:438. In one embodiment, the antibody comprises a heavy chain having an amino acid sequence SEQ ID NO:437. In another embodiment, the antibody comprises a light chain having an amino acid sequence SEQ ID NO: 438. In one embodiment, the antibody comprises (i) a heavy chain having an amino acid sequence SEQ ID NO:437; and (ii) a light chain having an amino acid sequence SEQ ID NO:438.

In some embodiments, the VH CDR1, VH CDR2, VH CDR3, VL CDR1, VL CDR2, and VL CDR3 amino acid sequences are according to the Kabat numbering system. In other embodiments, the VH CDR1, VH CDR2, VH CDR3, VL CDR1, VL CDR2, and VL CDR3 amino acid sequences are according to the Chothia numbering system. In some embodiments, the VH CDR1, VH CDR2, VH CDR3, VL CDR1, VL CDR2, and VL CDR3 amino acid sequences are according to the AbM numbering system; In other embodiments, the VH CDR1, VH CDR2, VH CDR3, VL CDR1, VL CDR2, and VL CDR3 amino acid sequences are according to the Contact numbering system. In some embodiments, the VH CDR1, VH CDR2, VH CDR3, VL CDR1, VL CDR2, and VL CDR3 amino acid sequences are according to the IMGT numbering system.

In one embodiment, the antibody is a humanized antibody. In another embodiment, the antibody is a fully human antibody. In one embodiment, the antibody is an IgG antibody. In one embodiment, the IgG antibody is an IgG1, IgG2, IgG3, or IgG4 antibody. In one embodiment, the antibody comprises a kappa light chain. In one embodiment, the antibody comprises a lambda light chain. In one embodiment, the antibody is a monoclonal antibody. In one embodiment, the antibody is multivalent. In one embodiment, the antibody is a multispecific antibody.

In one embodiment, the antibody specifically binds to Sdc2. In one embodiment, the Sdc2 is present on the surface of an endothelial cell. In one embodiment, the Sdc2 is present on the surface of a neural cell.

In one aspect, provided is a nucleic acid encoding a Sdc2 antibody provided herein. In another aspect, provided is a vector comprising a nucleic acid encoding a Sdc2 antibody provided herein. In one aspect, provided is a host cell comprising a vector comprising a nucleic acid encoding a Sdc2 antibody provided herein. In another aspect, provided is a kit comprising a vector comprising a nucleic acid encoding a Sdc2 antibody provided herein. In yet another aspect, provided is a kit comprising an antibody provided herein. In certain embodiments, the kit further comprises a container. In certain embodiments, the kit further comprises packaging. In certain embodiments, the kit further comprises instructions for use.

In another aspect, provided is a pharmaceutical composition comprising an Sdc2 antibody provided herein, and a pharmaceutically acceptable carrier. In one aspect, provided is a method of producing the pharmaceutical composition, comprising combining the Sdc2 antibody with a pharmaceutically acceptable carrier to obtain the pharmaceutical composition.

In one aspect provided is a method of reducing vascular cell permeability (also referred to as vascular permeability herein), comprising contacting the vascular cells with an Sdc2 antibody provided herein. In one aspect, provided is a method of reducing endothelial cell permeability (also referred to as endothelial permeability herein), comprising contacting the endothelial cells with an Sdc2 antibody provided herein.

In one aspect, provided is a method of reducing VEGFA-induced endothelial cell permeability, comprising contacting the endothelial cells with an Sdc2 antibody provided herein, either before, during or after the endothelial cells are contacted with the VEGFA. In one embodiment, the endothelial cells are contacted with the Sdc2 antibody before contact with the VEGFA. In one embodiment, the endothelial cells are contacted with the Sdc2 antibody during contact with the VEGFA. In one embodiment, the endothelial cells are contacted with the Sdc2 antibody after contact with the VEGFA.

In one aspect, provided is a method reducing vascular permeability in a subject, comprising administering to the subject an effective amount of an Sdc2 antibody provided herein. In one aspect, provided is a method reducing vascular leakage in a subject, comprising administering to the subject an effective amount of an Sdc2 antibody provided herein. In one aspect, provided is a method reducing endothelial permeability in a subject, comprising administering to the subject an effective amount of an Sdc2 antibody provided herein. In certain embodiments of the methods provided herein, the subject has a disease caused all or in part by cells expressing Sdc2.

In a specific embodiment, the subject is a human. In certain embodiments, the subject is a subject in need thereof. In a specific embodiment, the subject has or is at risk of having an Sdc2-mediated disease or disorder. In certain embodiments, the subject has or is at risk of having a disease or disorder associated with vascular permeability. In certain embodiments, the subject has or is at risk of having a disease or disorder associated with vascular leakage. In certain embodiments, the subject has or is at risk of having a disease or disorder associated with endothelial permeability. Exemplary diseases and disorders are provided elsewhere herein and are contemplated in the provided methods.

In another aspect, provided herein is a method of preventing, treating, or modulating a disease caused all or in part by cells expressing Sdc2, comprising administering to the subject an effective amount of an Sdc2 antibody provided herein. In some embodiments, the cells are endothelial cells. In some embodiments, the cells are neural cells.

In certain embodiments of the various methods provided herein, the disease is associated with vascular permeability or vascular leakage. In one embodiment, the disease is associated with vascular permeability. In one embodiment, the disease is associated with vascular permeability. In one embodiment, the disease is an acute respiratory distress syndrome (ARDS). In one embodiment, the disease is a COVID-19-induced ARDS. In one embodiment, the disease is a hemorrhagic stroke. In one embodiment, the disease is an ischemic stroke. In one embodiment, the disease is a neurological disease in which the BBB is altered or disrupted. In one embodiment, the disease is Parkinson's Disease. In one embodiment, the disease is Alzheimer's disease. In one embodiment, the disease is Huntington's Disease. In one embodiment, the disease is a peripheral neuropathy. In one embodiment, the disease is a traumatic brain injury. In one embodiment, the disease is epilepsy. In one embodiment, the disease is multiple sclerosis. In one embodiment, the disease is a neovascular eye disease. In one embodiment, the disease is a cardiovascular disease. In one embodiment, the disease is a myocardial infarction (also referred to herein as an acute myocardial infarction (AMI)). In one embodiment, the disease is congestive heart failure. In one embodiment, the disease is a blunt trauma injury. In one embodiment, the disease is a peripheral vascular disease. In one embodiment, the disease is a lymphedema. In one embodiment, the disease is POEMS (Polyneuropathy, Organomegaly, Endocrinopathy, Monoclonal plasma cell disorder, Skin changes) Syndrome. In one embodiment, the disease is a pediatric capillary leak syndrome. In one embodiment, the disease is an adult capillary leak syndrome. In one embodiment, the disease is a hydrocephalus. In one embodiment, the disease is a lymphedema. In one embodiment, the disease is an inflammation-associated edema. In one embodiment, the disease is an inflammatory disease. In one embodiment, the disease is systemic lupus erythematosus. In one embodiment, the disease is a rheumatoid arthritis cardiovascular disease. In one embodiment, the disease is a neovascular eye disease. In one embodiment, the disease is AMD. In one embodiment, the disease is diabetic retinopathy. In one embodiment, the disease is a stroke. In one embodiment, the disease is an ischemic stroke. In one embodiment, the disease is a hemorrhagic stroke. In one embodiment, the disease is a cancer.

In another aspect, provided is a method of treating a stroke in a subject, comprising administering to the subject an effective amount of a Sdc2 antibody provided herein. In another aspect, provided is a method of treating an ischemic stroke in a subject, comprising administering to the subject an effective amount of a Sdc2 antibody provided herein. In some embodiments, the subject a brain lesion area associated with the ischemic stroke. In one embodiment, the administration of the antibody results in a reduction of endothelial cell permeability in or surrounding the brain lesion area. In one embodiment, the endothelial cell permeability is VEGFA-induced endothelial cell permeability. In one embodiment, the administration of the antibody results in a reduction of vascular permeability in or surrounding the brain lesion area. In one embodiment, the administration of the antibody results in a reduction in the size of a penumbra of the brain lesion area. In one embodiment, the administration of the antibody results in a reduction in the size of an edema of the brain lesion area. In one embodiment, the administration of the antibody results in a reduction in the size of an infarct of the brain lesion area. In one embodiment, the reduction in size is detected by MRI and/or measured in area or volume. In another aspect, provided is a method of treating a hemorrhagic stroke in a subject, comprising administering to the subject an effective amount of a Sdc2 antibody provided herein.

In another aspect, provided is a method of reducing eye inflammation is a subject, comprising administering to the subject an effective amount of an Sdc2 antibody provided herein. In some embodiments, the administration of the antibody results in a reduction of one or more inflammation marker in the choroid of an eye of the subject. In one embodiment, the inflammation marker is CD31 or F4/80. In one embodiment, the administration of the antibody results in substantially the same expression of one or more endothelial marker in the choroid of an eye of the subject. In one embodiment, the endothelial marker is ERG. In another aspect, provided is a method of treating a neovascular eye disease in a subject, comprising administering to the subject an effective amount of a Sdc2 antibody provided herein. In another aspect, provided is a method of treating diabetic retinopathy in a subject, comprising administering to the subject an effective amount of a Sdc2 antibody provided herein. In another aspect, provided is a method of treating AMD in a subject, comprising administering to the subject an effective amount of the antibody of a Sdc2 antibody provided herein. In one embodiment, the administration of the antibody results in a reduction of the central retinal thickness of an eye of the subject. In one embodiment, the reduction in central retinal thickness is measured by optical coherence tomography (OCT). In one embodiment, the administration of the antibody results in a reduction of endothelial permeability in an eye fundus tissue of the subject. In one embodiment, the endothelial permeability is VEGFA-induced endothelial cell permeability. In one embodiment, the endothelial permeability is measured by fundus fluorescein angiography (FFA). In one embodiment, the administration of the antibody results in a reduction of vascular permeability in an eye fundus tissue of the subject. In one embodiment, the vascular permeability is measured by FFA. In one embodiment, the administration of the antibody results in an upregulation of Dep-1 surface expression on cells in an eye fundus tissue of the subject. In one embodiment, the administration of the antibody results in an enhancement of dephosphorylation of vascular endothelial growth factor receptor 2 (VEGFR2) protein at residue Y951 in cells in an eye fundus tissue of the subject. In one embodiment, the administration of the antibody results in a reduction of inflammation in an eye fundus tissue of the subject. In one embodiment, the administration of the antibody results in a reduction in the expression of one or more inflammatory marker in an eye fundus tissue of the subject. In one embodiment, the inflammatory marker is selected from pro-inflammatory cytokines and immune cell surface proteins. In one embodiment, the inflammatory marker is F4/80. In one embodiment, the administration of the antibody results in no change in angiogenesis in an eye fundus tissue of the subject. In one embodiment, the angiogenesis is choroidal neovascularization (CNV). In one embodiment, the administration of the antibody results in substantially no change in expression of one or more endothelial marker in an eye fundus tissue of the subject. In one embodiment, the endothelial marker is ERG. In one embodiment, the endothelial marker is CD31. In one embodiment, the eye fundus tissue is the retina of the eye. In one embodiment, the eye fundus tissue is the macula of the yet. In one embodiment, the eye fundus tissue is the choroid of the eye. In one embodiment, upon administering of the antibody, the vision acuity of the subject is enhanced. In one embodiment, the subject is a human suffering from or at risk of developing AMD.

In another aspect, provided is a method of treating a cardiovascular disease in a subject, comprising administering to the subject an effective amount of a Sdc2 antibody provided herein. In another aspect, provided is a method of treating congestive heart failure in a subject, comprising administering to the subject an effective amount of a Sdc2 antibody provided herein. In another aspect, provided is a method of treating a myocardial infarction in a subject, comprising administering to the subject an effective amount of the antibody of a Sdc2 antibody provided herein. In one embodiment, the administration of the antibody results in a reduction of endothelial permeability in a heart tissue of the subject. In one embodiment, the endothelial permeability is VEGFA-induced endothelial cell permeability. In one embodiment, the endothelial permeability is measured by an Evans Blue assay or a Dextran perfusion assay. In one embodiment, the administration of the antibody results in a reduction of vascular permeability in a heard tissue of the subject. In one embodiment, the vascular permeability is measured by an Evans Blue assay or a Dextran perfusion assay. In one embodiment, the administration of the antibody results in an upregulation of Dep-1 surface expression on cells in a heart tissue of the subject. In one embodiment, the administration of the antibody results in an enhancement of dephosphorylation of vascular endothelial growth factor receptor 2 (VEGFR2) protein at residue Y951 in cells in a heart tissue of the subject. In one embodiment, the administration of the antibody results in a reduction of inflammation in a heart tissue of the subject. In one embodiment, the administration of the antibody results in a reduction in the expression of one or more inflammatory marker in a heart tissue of the subject. In one embodiment, the inflammatory marker is selected from pro-inflammatory cytokines and immune cell surface proteins. In one embodiment, the inflammatory marker is CD11b, GM-CSF, MIG, CCL11, IL-3, IL-6, or TNF-α. In one embodiment, reduction of expression of the one or more inflammatory marker occurred within about 24 hours, within about 36 hours, or within about 72 hours after administration of the antibody. In one embodiment, the administration of the antibody results in an enhancement of left ventricular (LV) ejection fraction (LVEF) of the heart of the subject. In one embodiment, the administration of the antibody results in an enhancement of cardiac output of the heart of the subject. In one embodiment, the administration of the antibody results in a reduction of LV end diastolic diameter (LVEDD) of the heart of the subject. In one embodiment, the administration of the antibody results in a reduction of LV end systolic diameter (LVESD) of the heart of the subject. In one embodiment, the administration of the antibody results in a reduction of LV end diastolic volume of the heart of the subject. In one embodiment, the administration of the antibody results in a reduction of LV end systolic volume of the heart of the subject. In one embodiment, the administration of the antibody results in a reduction of LV mass of the heart of the subject. In one embodiment, the administration of the antibody results in an enhancement of fractional shortening of the heart of the subject. In one embodiment, the administration of the antibody results in an enhancement of ejection fraction of the heart of the subject. In one embodiment, the administration of the antibody results in a reduction of risk or duration of post-infarct ventricular tachycardia (VT) of the subject. In one embodiment, the post-infarct VT of the subject is measured by an electrocardiogram with programmed stimulation of the heart of the subject. In one embodiment, an increase in the number of stimuli for inducing the VT indicates a reduced risk of post-infarct VT in the subject. In one embodiment, a reduced duration of induced VT under a hypokalemic condition indicates a reduced risk of post-infarct VT in the subject; optionally wherein a longer cycle length of induced VT indicates a reduced risk of post-infarct VT in the subject. In one embodiment, the administration of the antibody results in a reduction of risk for the subject to have a heart failure. In one embodiment, the risk is having the heart failure within 1-3 months following the myocardial infarction. In one embodiment, the subject is a human suffering from or at risk of developing AMI

In some embodiments of the methods provided herein, the subject is a human. In certain embodiments of the methods provided herein, the subject is a human subject in need thereof.

### 5. BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is a table illustrating a list of mAbs of the invention with summary of affinity against human Sdc2 (Kd) and functional activity as inhibition of VEGFA-induced vascular permeability in vitro.

FIGS. 2A-2B illustrate clinical chemistry results from a toxicology study of clone 20-H19-AB in non-human primates. Graphs represent the counts of red and white blood cell populations (FIG. 2A), as well as platelets and hemoglobin (FIG. 2B).

FIGS. 3A-3B illustrate clinical chemistry results from a toxicology study of clone 20-H19-AB in non-human primates. Graphs represent the levels of glucose and total triglycerides (FIG. 3A), and total cholesterol and total CO2 (FIG. 3B).

FIG. 4 illustrates clinical chemistry results from a toxicology study of clone 20-H19-AB in non-human primates. Graphs represent the levels of fibrinogen, c-reactive protein (CRP), and amylase.

FIG. 5 illustrates clinical chemistry results from a toxicology study of clone 20-H19-AB in non-human primates. Graphs represent the levels of markers of liver function including ALT, AST, and bilirubin.

FIG. 6 illustrates clinical chemistry results from a toxicology study of clone 20-H19-AB in non-human primates. Graphs represent markers of kidney function including creatinine (CRE) and blood urea nitrogen (BUN).

FIG. 7 illustrates the clinical chemistry results of a Miles toxicology assay in non-human primates using clone 20-H19-AB 24 hrs. after mAb injection.

FIG. 8 illustrates functional assays of the 8-G17 antibody clone.

FIG. 9 illustrates assays determining the binding affinity of the 8-G17 antibody clone.

FIG. 10 illustrates functional assays of the 20-H19-AB antibody clone.

FIG. 11 illustrates assays determining the binding affinity of the 20-H19-AB antibody clone.

FIG. 12 illustrates functional assays of the 20-H19-AB antibody clone using non-human primate cells.

FIG. 13 illustrates functional assays of the R3-P3-C11 antibody clone.

FIG. 14 illustrates assays determining the binding affinity of the R3-P3-C11 antibody clone.

FIG. 15 illustrates a functional assay of the R4M-P3-E06 antibody clone.

FIG. 16 illustrates an assay determining the binding affinity of the R4M-P3-E06 antibody clone.

FIG. 17 illustrates a functional assay of the R3-P3-E09 antibody clone.

FIG. 18 illustrates an assay determining the binding affinity of the R3-P3-E09 antibody clone.

FIG. 19 illustrates functional assays of the R3-P1-C02 antibody clone.

FIG. 20 illustrates assays determining the binding affinity of the R3-P1-C02 antibody clone.

FIG. 21 illustrates functional assays of the R3-P3-A12 antibody clone.

FIG. 22 illustrates assays determining the binding affinity of the R3-P3-A12 antibody clone.

FIG. 23 illustrates functional assays of the R4M-P3-A12 antibody clone.

FIG. 24 shows assays determining the binding affinity of the R4M-P3-A12 antibody clone.

FIG. 25 illustrates functional assays of the R4M-P1-A10 antibody clone.

FIG. 26 shows assays determining the binding affinity of the R4M-P1-A10 antibody clone.

FIG. 27 illustrates a table listing the antibody clones used in epitope binning studies.

FIG. 28 illustrates a raw sensorgram of the epitope binning studies. The saturating antibodies were 19838-10-I12-A 1983-20-H19-A.

FIG. 29 illustrates a matrix of raw blocking data from the epitope binning studies. Clones that did not show binding to the antigen in the saturation step are highlighted in grey (19838-20H19-AA, 19844-R3-P1-E07, 19844-R4M-P1-B01). Since these clones did not bind to antigen significantly in this assay, they were removed in both orientations. Clones with extremely fast off-rates (19844-R3-P1-C02, 19844-R4M-P1-A10, 19844-R4M-P3-A12) are highlighted in darker grey. Since saturation is not possible to achieve saturation with these clones, they are removed as ligands, but retained in the analyte orientation in the analysis.

FIG. 30 illustrates the epitope binning of 18 antibodies against huSdc2.

FIG. 31 illustrates an epitope binning clustergram. Antibody clusters share similar but not necessarily identical competition profiles.

FIGS. 32A-32B illustrate the target regions of Sdc2. FIG. 32A is a diagram of the human Sdc2 protein with the region of the extracellular domain representing the DEP1-binding region highlighted. Below is the amino acid sequence of a portion of the Sdc2 protein with the “AG3 peptide” corresponding to the DEP1-binding region highlighted. FIG. 32B is an alignment of the AG3 peptide regions from human (top), mouse (middle), and porcine (bottom).

FIG. 33 shows the VE-cadherin staining pattern in cell cultures treated with VEGFA in the absence (left panel) or presence (right panel) of the anti-Sdc2 antibody.

FIG. 34 is a schematic illustration of the pathologic mechanism of vascular leakage and edema.

FIG. 35 shows the MCA occultation procedure used to establish the disease model in non-human primates.

FIG. 36 shows the quantification of infarct size using 2D FLAIR sequences in each plane by AMIRA software.

FIG. 37 shows the quantification of change in stroke volume relative to sham in mm3 1 day and 3 days post treatment.

FIG. 38 shows exemplary MRI image construction of testing subjects receiving the vehicle and the anti-Sdc2 antibody.

FIGS. 39A-39D illustrate intravitreal injection or systemic injection of a polyclonal rabbit anti-mouse Sdc2 antibody (Ab3) were effective in reducing vascular leakage and lesion volume in an AMD mouse model. FIG. 39A shows Fundus fluorescein angiography (FFA) images taken 7 days after laser photocoagulation from mice that received intravitreal or systemic injection of Ab3, an anti-VEGF antibody or vehicle control on day 3 and day 6 after photocoagulation. FIG. 39B shows quantification of vascular leakage in lesion sites at day 7 (end of the experiment). FIG. 39C shows optical coherence tomography (OCT) images taken 7 days after laser photocoagulation from mice received intravitreal or systemic injection of anti-Sdc2 antibody, anti-VEGF antibody or vehicle control on day 3 and day 6 after photocoagulation. FIG. 39D shows quantification of the lesion volume at day 7 (end of the experiment).

FIG. 40 shows electroretinography (ERG) results from mice that received intravitreal or systemic injection of anti-Sdc2 antibody Ab3, anti-VEGF antibody or vehicle control on day 3 and day 6 after photocoagulation. As shown, the A-wave and B-wave amplitudes of control mice and mice injected with the anti-Sdc2 antibody at different light intensities had no significant difference, indicating that injection of anti-Sdc2 antibody did not result in retinal disfunction.

FIGS. 41A-41D shows illustrate both intravitreal and systemic injections of an anti-Sdc2 antibody Ab3 inhibited inflammatory infiltration. FIG. 41A shows immunofluorescence microscopy images of dissected flat mount of the mouse choroid/retinal pigment epithelium (RPE) stained to visualize CD31, ERG and F4/80 expression in mice received intravitreal or systemic injections of anti-Sdc2 antibody or anti-VEGF antibody. FIGS. 41B-41D show quantifications of the positive areas of CD31, ERG and F4/80, respectively.

FIG. 42 illustrates that an anti-Sdc2 antibody inhibited VEGFA-induced permeability in endothelial cells (ECs). Y-axis shows the strength of cell adhesion which is represented as the Delta Cell Index (unit-less measurement). X-axis shows the time (in hour) after VEGFA or anti-Sdc2 antibodies (Ab3) were added to the endothelial cell culture.

FIGS. 43A-43J illustrate treatment with a polyclonal anti-Sdc2 antibody (Ab3) that binds to the Dep-1 binding region in Sdc2 prevented the progression of post-myocardial infarction heart failure in a mouse myocardial infarction (MI) model. The MI model was created by surgically occluding the left anterior descending coronary artery (LAD) with a suture for 1 hour to induce ischemia, followed by removing the suture to restore blood flow and tissue reperfusion, sometimes referred to as the ischemia-reperfusion (IR) surgery. The MI model mice were treated with a single dose of 4 mg/kg body weight anti-Sdc2 antibody Ab3 administered intravenously through the tail veins immediately following the reperfusion. A control group of mice received equivalent amount of IgG instead of the anti-Sdc2 antibody. Cardiac functions of mice thus treated were measured 1 day, 7 days, 14 days, and 1 month after the IR surgery using echocardiography (cardiac ultrasound). FIG. 43A: post-infarct left ventricular (LV) function (LVEF) (%); FIG. 43B: cardiac output (ml/min); FIG. 43C: end diastolic LV internal diameter (LVIDd) (mm); FIG. 43D: end systolic LV internal diameter (LVIDs) (mm); FIG. 43E: ejection fraction (%); FIG. 43F: fractional shortening (%); FIG. 43G: LV mass (mg); FIG. 43H: stroke volume (μL); FIG. 43I: LV end diastolic volume (μL); FIG. 43J: LV end systolic volume (μL). Dotted lines represent values for normal mice before surgery (from literature).

FIGS. 44A-44D illustrate treatment with a polyclonal anti-Sdc2 antibody (Ab3) decreased the infarct size and infection in the heart of MI model mice. FIG. 44A shows Masson's trichrome staining images of hearts of MI model mice treated with Ab3 or IgG control, which images were taken one month after the IR surgery. Each row shows a serial of cross-section images of a mouse's heart at different sectional depths. As shown, the heart infarct sizes were visually smaller in the group received Ab3 treatment as compared to the group received the IgG control. FIG. 44B shows the immunohistochemistry staining visualizing CD11b, a macrophage marker, in the heart tissues of MI mice treated with Ab3 or IgG, which images were taken one month after the IR surgery. As shown, macrophage infiltration in the heart tissues was reduced in the group received Ab3 treatment as compared to the group received the IgG control, indicating the levels of tissue damage and inflammation in the heart tissues were reduced following the anti-Sdc2 treatment. FIG. 44C shows 2,3,5-Triphenyltetrazolium chloride (TTC) staining images of hearts of MI model mice treated with Ab3 or IgG control, which images were taken 24 hours after the IR surgery and FIG. 44D shows the quantification of the infarct size from this study. As shown, the heart infarct size was significantly smaller in the group received Ab3 treatment as compared to the group received the IgG control.

FIGS. 45A-45B illustrate treatment with a polyclonal anti-Sdc2 antibody (Ab3) reduced endothelial permeability in heart tissues of MI model mice 24 and 72 hours after MI by the IR surgery. Particularly, FIG. 45A shows quantitation of endothelial permeability measured using the FITC-dextran permeability assay 24 hours after the PI surgery. Mice were given an IV injection of FITC-dextran (70 Kda molecular weight) at 24 hrs post-surgery and then euthanized after 1 hour from dextran injection. Mice were then perfused with 30 ml PBS and heart samples were collected and lysed in order to quantify FITC-dextran extravasation (with fluorescence plate reader). As shown, endothelial permeability in mice heart tissues was significantly reduced in the group received Ab3 treatment as compared to the group received the IgG control. FIG. 45B shows images and quantitation of vascular leakage measured using Evans blue. Mice were given intravenous injections through tail veins Evans Blue that binds albumin after 72 from surgery and allowed to circulate for 1 hr before to allow extravasation. The upper panel shows actual photos of hearts of MI mice taken at 72 hours post-surgery and after Evans blue injection. Vascular leakage in the heart was observed when blood vessels started to leak protein and thus, also the Evans blue that is bound to albumin, resulting in a bluish coloration of the heart tissues. The lower panel shows quantitation of vascular leakage measured from areas with infarct risk and rest myocardium in this study (Y-axis indicates the amount of leaked Evans blue).

FIGS. 46A-46C illustrate treatment with a polyclonal anti-Sdc2 antibody (Ab3) significantly reduced the induction of post-infarct ventricular tachycardia (VT) in MI mice. FIG. 46A shows VT durations of mice treated with Ab3 or IgG under normokalemia and hypokalemia conditions. FIG. 46B shows mice treated with Ab3 did not have prolonged action potential duration (APD) under hypokalemia condition in contrast to mice treated with IgG. FIG. 46C shows more benign arrhythmias were induced in mice treated with Ab3 as compared to mice treated with IgG. These data demonstrate that anti-Sdc2 antibody treatment significantly increased the threshold for VT induction. Particularly, increased number of stimuli was needed to induce VT or fibrillation (VF) in the anti-Sdc2 antibody treatment group, and the VT cycle length of the induced VT was longer after programmed stimulation was observed for the anti-Sdc2 antibody treatment group. These animals were less prone to post-infarct ventricular arrhythmias and demonstrated more benign arrhythmias.

FIG. 47 shows plasma levels (pg/ml) of various biomarkers measured with ELISA 24 to 72 hours after the episode of induced MI in groups of mice that received the anti-Sdc2 antibody (Ab3) treatment or IgG control. As show, GM-CSF, MIG, Eotaxin (CCL11), IL-3, IL-6, TNF-α, and MCP1 (CCL2) were identified as systemic biomarkers for AMI treatment.

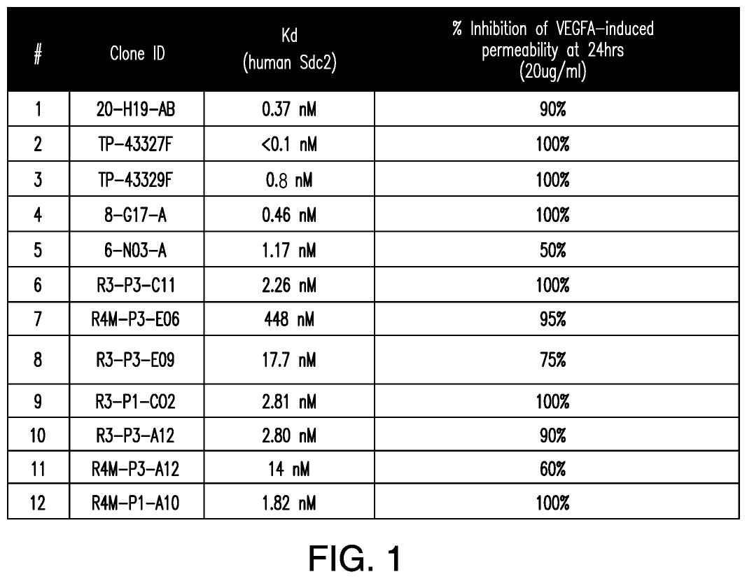

### 6. DETAILED DESCRIPTION

### 6.1 Definitions

Various publications, articles and patents are cited or described in the background and throughout the specification. All applications, publications, patents and other references, GenBank citations and ATCC citations cited herein are incorporated by reference in their entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only and is not intended to be limiting. In case of conflict, the specification, including definitions, will control.

The articles “a,” “an,” and “the” are used herein to refer to one or to more than one (i.e., to at least one) of the grammatical object of the article. That is, these terms include plural referents unless the context clearly indicates otherwise. By way of example, “an element” means one element or more than one element.

The terms “about” and “approximately” as used herein when referring to a measurable value such as an amount, a temporal duration, and the like, mean within 20%, within 15%, within 10%, within 9%, within 8%, within 7%, within 6%, within 5%, within 4%, within 3%, within 2%, within 1%, within 0.1% or less of a given value or range.

A disease or disorder is “alleviated” if the severity of a symptom of the disease or disorder, the frequency with which such a symptom is experienced by a patient, or both, is reduced.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.” In addition, unless expressly stated to the contrary, “or” refers to an inclusive or and not to an exclusive or. For example, a condition A or B is satisfied by any one of the following: A is true (or present), and B is false (or not present), A is false (or not present), and B is true (or present), and both A and B are true (or present).

The term “antibody,” as used herein, refers to an immunoglobulin molecule which specifically binds with an antigen. Antibodies can be intact immunoglobulins derived from natural sources or from recombinant sources and can also be immunoreactive portions or immunoreactive fragments of intact immunoglobulins. The terms “antibody,” “immunoglobulin,” or “Ig” are used interchangeably herein, and is used in the broadest sense and specifically covers, for example, individual anti-Sdc2 monoclonal antibodies (including agonist, antagonist, neutralizing antibodies, full length or intact monoclonal antibodies), anti-Sdc2 antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain anti-Sdc2 antibodies, and fragments of anti-Sdc2 antibodies, as described below. An antibody can be human, humanized, chimeric and/or affinity matured, as well as an antibody from other species, for example, mouse and rabbit, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). In specific embodiments, the specific molecular antigen can be bound by an antibody provided herein, including a Sdc2 polypeptide, a Sdc2 fragment, or a Sdc2 epitope. Antibodies also include, but are not limited to, synthetic antibodies, recombinantly produced antibodies, camelized antibodies, intrabodies, anti-idiotypic (anti-Id) antibodies, and functional fragments (e.g., antigen-binding fragments such as Sdc2-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments such as Sdc2-binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabody, tetrabody, and minibody. In particular, antibodies provided herein include immunoglobulin molecules and immunologically active portions of immunoglobulin molecules, for example, antigen-binding domains or molecules that contain an antigen-binding site that binds to a Sdc2 antigen (e.g., one or more CDRs of an anti-Sdc2 antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Pluckthun and Skerra, 1989, Meth. Enzymol. 178:497-515; and Day, Advanced Immunochemistry (2d ed. 1990). Anti-Sdc2 antibodies may be agonistic antibodies or antagonistic antibodies. Described herein are antagonistic antibodies to Sdc2, including antibodies that inhibits Sdc2 activity.

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies provided herein can be of any of the five major classes or corresponding sub-classes. In specific embodiments, the antibodies provided herein are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies provided herein can, in certain embodiments, contain a kappa light chain constant domain. The antibodies provided herein can, in certain embodiments, also contain a lambda light chain constant domain. According to particular embodiments, the antibodies provided herein include heavy and/or light chain constant regions from rat or human antibodies. In specific embodiments, the constant region is a human constant region.

In addition to the heavy and light constant domains, antibodies contain an antigen-binding region that is made up of a light chain variable region (VL) and a heavy chain variable region (VH), each of which contains three domains (i.e., complementarity determining regions 1 (CDR1), CDR2 and CDR3. A “CDR” refers to one of three hypervariable regions (HCDR1, HCDR2 or HCDR3) within the non-framework region of the immunoglobulin (Ig or antibody) VH B-sheet framework, or one of three hypervariable regions (LCDR1, LCDR2 or LCDR3) within the non-framework region of the antibody VL β-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains (Kabat et al., J. Biol. Chem. 252:6609-6616 (1977); Kabat, Adv. Prot. Chem. 32:1-75 (1978)). CDR region sequences also have been defined structurally by Chothia as those residues that are not part of the conserved β-sheet framework, and thus are able to adapt different conformations (Chothia and Lesk, J. Mol. Biol. 196:901-917 (1987)). Both terminologies are well recognized in the art. CDR region sequences have also been defined by AbM, Contact and IMGT. Exemplary CDR region sequences are illustrated herein, for example, in the tables provided in the Examples below. The positions of CDRs within a canonical antibody variable region have been determined by comparison of numerous structures (Al-Lazikani et al., J. Mol. Biol. 273:927-948 (1997); Morea et al., Methods 20:267-279 (2000)). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra (1997)). Such nomenclature is similarly well known to those skilled in the art.

The light chain variable region CDR1 domain is interchangeably referred to herein as LCDR1 or VL CDR1. The light chain variable region CDR2 domain is interchangeably referred to herein as LCDR2 or VL CDR2. The light chain variable region CDR3 domain is interchangeably referred to herein as LCDR3 or VL CDR3. The heavy chain variable region CDR1 domain is interchangeably referred to herein as HCDR1 or VH CDR1. The heavy chain variable region CDR2 domain is interchangeably referred to herein as HCDR2 or VH CDR2. The heavy chain variable region CDR1 domain is interchangeably referred to herein as HCDR3 or VH CDR3.

The terms “hypervariable region,” “HVR,” or “HV,” such as a VH or VL, when used herein refers to the regions of an antibody variable region that are hypervariable in sequence and/or form structurally defined loops. Generally, antibodies comprise six hypervariable regions: three in the VH (HCDR1, HCDR2, HCDR3), and three in the VL (LCDR1, LCDR2, LCDR3). A number of hypervariable region delineations are in use and are encompassed herein. The “Kabat” CDRs are based on sequence variability and are the most commonly used (see, e.g., Kabat et al., Sequences of Proteins of Immunological Interest, 5th Ed. Public Health Service, National Institutes of Health, Bethesda, MD. (1991)). “Chothia” refers instead to the location of the structural loops (see, e.g., Chothia and Lesk, J. Mol. Biol. 196:901-917 (1987)). The end of the Chothia CDR-HCDR1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The “AbM” hypervariable regions represent a compromise between the Kabat CDRs and Chothia structural loops and are used by Oxford Molecular's AbM antibody modeling software (see, e.g., Martin, in Antibody Engineering, Vol. 2, Chapter 3, Springer Verlag). “Contact” hypervariable regions are based on an analysis of the available complex crystal structures.

Recently, a universal numbering system has been developed and widely adopted, ImMunoGeneTics (IMGT) Information System® (Lafranc et al., Dev. Comp. Immunol. 27(1):55-77 (2003)). IMGT is an integrated information system specializing in immunoglobulins (IG), T cell receptors (TR) and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues and are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309:657-670 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). An Exemplary system, shown herein, combines Kabat and Chothia.

| | Exemplary | IMGT | Kabat | AbM | Chothia | Contact |
|---|---|---|---|---|---|---|
| V_{H} CDR1 | 26-35 | 27-38 | 31-35 | 26-35 | 26-32 | 30-35 |
| V_{H} CDR2 | 50-65 | 56-65 | 50-65 | 50-58 | 53-55 | 47-58 |
| V_{H} CDR3 | 95-102 | 105-117 | 95-102 | 95-102 | 96-101 | 93-101 |
| V_{L} CDR1 | 24-34 | 27-38 | 24-34 | 24-34 | 26-32 | 30-36 |
| V_{L} CDR2 | 50-56 | 56-65 | 50-56 | 50-56 | 50-52 | 46-55 |
| V_{L} CDR3 | 89-97 | 105-117 | 89-97 | 89-97 | 91-96 | 89-96 |

Hypervariable regions may comprise “extended hypervariable regions” as follows: 24-36 or 24-34 (LCDR1), 46-56 or 50-56 (LCDR2) and 89-97 or 89-96 (LCDR3) in the VL and 26-35 or 26-35A (HCDR1), 50-65 or 49-65 (HCDR2) and 93-102, 94-102, or 95-102 (HCDR3) in the VH. CDR sequences, reflecting each of the above numbering schemes, are provided herein, including in the tables in the Examples section below.

The term “constant region” or “constant domain” refers to a carboxy terminal portion of the light and heavy chain which is not directly involved in binding of the antibody to antigen but exhibits various effector function, such as interaction with the Fc receptor. The terms refer to the portion of an immunoglobulin molecule having a more conserved amino acid sequence relative to the other portion of the immunoglobulin, the variable region, which contains the antigen binding site. The constant region may contain the CH1, CH2 and CH3 regions of the heavy chain and the CL region of the light chain.

The term “framework” or “FR” residues are those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a β sheet configuration, connected by three hypervariable regions, which form loops connecting, and in some cases form part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering as in Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon, as described above.

As used herein, the term “monoclonal antibody” refers to an antibody obtained from a population of substantially homogeneous antibodies, i.e., the individual antibodies comprising the population are identical except for possible naturally occurring mutations that can be present in minor amounts. Monoclonal antibodies provided herein can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, the term “antigen-binding fragment” refers to an antibody fragment such as, for example, a diabody, a Fab, a Fab′, a F(ab′)2, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), a single domain antibody (sdAb) an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment bind. According to particular embodiments, the antigen-binding fragment comprises a light chain variable region, a light chain constant region, and an Fd segment of the heavy chain. According to other particular embodiments, the antigen-binding fragment comprises Fab and F (ab′). In specific embodiments, the antigen binding fragment will exhibit at least one if not some or all of the biological functions attributed to the intact antibody, the function comprising at least binding to the target antigen (e.g., a Sdc2 binding fragment or fragment that binds to Sdc2).

As used herein, the term “single-chain antibody” refers to a conventional single-chain antibody in the field, which comprises a heavy chain variable region and a light chain variable region connected by a short peptide of about 15 to about 20 amino acids. As used herein, the term “single domain antibody” refers to a conventional single domain antibody in the field, which comprises a heavy chain variable region and a heavy chain constant region or which comprises only a heavy chain variable region.

As used herein, the term “multispecific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes do not overlap or do not substantially overlap. In an embodiment, the first and second epitopes are on different antigens, e.g., the different proteins (or different subunits of a multimeric protein). In an embodiment, a multispecific antibody comprises a third, fourth, or fifth immunoglobulin variable domain. In an embodiment, a multispecific antibody is a bispecific antibody molecule, a trispecific antibody molecule, or a tetraspecific antibody molecule.

As used herein, the term “bispecific antibody” refers to a multispecific antibody that binds no more than two epitopes or two antigens. A bispecific antibody is characterized by a first immunoglobulin variable domain sequence which has binding specificity for a first epitope (e.g., an epitope on a Sdc2 antigen) and a second immunoglobulin variable domain sequence that has binding specificity for a second epitope. In an embodiment, the first and second epitopes are on different antigens, e.g., the different proteins (or different subunits of a multimeric protein). In an embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody comprises a half antibody, or fragment thereof, having binding specificity for a first epitope and a half antibody, or fragment thereof, having binding specificity for a second epitope. In an embodiment, a bispecific antibody comprises a scFv, or fragment thereof, having binding specificity for a first epitope, and a scFv, or fragment thereof, having binding specificity for a second epitope.

The terms “antibodies that specifically bind to Sdc2,” “antibodies that specifically bind to a Sdc2 epitope,” and analogous terms are also used interchangeably herein and refer to antibodies that specifically bind to a Sdc2 polypeptide, such as a Sdc2 antigen, or fragment, or epitope (e.g., human Sdc2 such as a human Sdc2 polypeptide, antigen, or epitope). An antibody that specifically binds to Sdc2 (e.g., human Sdc2) may bind to the extracellular domain or peptide derived from the extracellular domain of Sdc2. An antibody that specifically binds to Sdc2 (e.g., human Sdc2) may bind to the Dep-1 binding region in Sdc2. An antibody that specifically binds to a Sdc2 antigen (e.g., human Sdc2) may be cross-reactive with related antigens (e.g., cyno Sdc2). In certain embodiments, an antibody that specifically binds to a Sdc2 antigen does not cross-react with other antigens. An antibody that specifically binds to a Sdc2 antigen can be identified, for example, by immunoassays, Biacore®, or other techniques known to those of skill in the art. An antibody binds specifically to a Sdc2 antigen when it binds to a Sdc2 antigen with higher affinity than to any cross-reactive antigen as determined using experimental techniques, such as radioimmunoassays (RIA) and enzyme linked immunosorbent assays (ELISAs). Typically, a specific or selective reaction will be at least twice background signal or noise and may be more than 10 times background. See, e.g., Fundamental Immunology 332-36 (Paul ed., 2d ed. 1989) for a discussion regarding antibody specificity. An antibody which “binds an antigen of interest” (e.g., a target antigen such as Sdc2) is one that binds the antigen with sufficient affinity such that the antibody is useful as a therapeutic agent in targeting a cell or tissue expressing the antigen and does not significantly cross-react with other proteins. In such embodiments, the extent of binding of the antibody to a “non-target” protein will be less than about 10% of the binding of the antibody to its particular target protein, for example, as determined by fluorescence activated cell sorting (FACS) analysis or RIA. With regard to the binding of an antibody to a target molecule, the term “specific binding,” “specifically binds to,” or “is specific for” a particular polypeptide or an epitope on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “anti-Sdc2 antibody” or “an antibody that binds to Sdc2” includes an antibody that is capable of binding Sdc2 with sufficient affinity such that the antibody is useful, for example, as a diagnostic agent in targeting Sdc2. The term “specific binding,” “specifically binds to,” or “is specific for” a particular polypeptide or an epitope on a particular polypeptide target as used herein refers to binding where a molecule binds to a particular polypeptide or epitope on a particular polypeptide without substantially binding to any other polypeptide or polypeptide epitope. In certain embodiments, an antibody that binds to Sdc2 has a dissociation constant (K_{D}) of less than or equal to 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In certain embodiments, anti-Sdc2 antibody binds to an epitope of Sdc2 that is conserved among Sdc2 from different species (e.g., between human and cyno Sdc2). In certain embodiments, an antibody that “specifically binds to Sdc2” refers to an antibody that binds to a Sdc2, preferably a human Sdc2, with a KD of 1×10⁻⁷ M or less, such as 1×10⁻⁸ M or less, 5×10⁻⁹ M or less, 1×10⁻⁹ M or less, 5×10⁻¹⁰ M or less, or 1×10⁻¹⁰ M or less.

The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antibody can be determined by using surface plasmon resonance, such as by using a biosensor system, e.g., a Biacore® system, or by using bio-layer interferometry technology, such as an Octet RED96 system. The smaller the value of the KD of an antibody, the higher affinity that the antibody binds to a target antigen.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions. In this regard, whether the antibody is an antibody fragment or an intact immunoglobulin, an antibody comprises variable regions, including a heavy chain variable region and a light chain variable region, which determine antigenicity. Antibodies are typically tetramers of immunoglobulin molecules. Antibodies may exist in a variety of forms including, for example, polyclonal antibodies, monoclonal antibodies, Fv, Fab, and F (ab)₂, as well as single chain antibodies (scFv) and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426). Examples of antibody fragments include, but are not limited to, Fab, Fab′, F(ab′)2, and Fv fragments, linear antibodies, scFv antibodies, and multispecific antibodies formed from antibody fragments.

The term “anti-syndecan-2 antibody,” “Sdc2 antibody” and related terms refer to an antibody that specifically binds to syndecan-2 under physiological conditions.

An “antibody heavy chain,” as used herein, refers to the larger of the two types of polypeptide chains present in all antibody molecules in their naturally occurring conformations. The antibody heavy chain comprises the heavy chain variable region and the heavy chain constant region.

An “antibody light chain,” as used herein, refers to the smaller of the two types of polypeptide chains present in all antibody molecules in their naturally occurring conformations. a and B light chains refer to the two major antibody light chain isotypes. The antibody light chain comprises the light chain variable region and the light chain constant region. Together, the light chain variable region(s) and the heavy chain variable region(s) of an antibody determine the antigenicity of the antibody.

By the term “synthetic antibody” as used herein, is meant an antibody which is generated using recombinant DNA technology, such as, for example, an antibody expressed by a CHO cells as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “coronavirus disease 2019” (COVID-19), as used herein, refers to the disease caused initially by infection of a subject with the novel 2019 coronavirus. The novel 2019 coronavirus is also known as severe acute respiratory syndrome coronavirus 2 (SARS-CoV-2). COVID-19, while caused initially by the infection with the SARS-CoV-2, is characterized in that it triggers a severe immune response in a subpopulation of individuals. The immune response to the SARS-CoV-2 virus and to the cells infected therefrom, in combination with the damage to the cells of the lung caused by the SARS-CoV-2 virus itself, can lead to acute respiratory distress syndrome in a subset of patients. COVID-19 can thereby require intubation, mechanical ventilation, and/or the use of a heart and lung bypass machine in a further subset of patients.

As used herein, the term “heterologous peptide” refers to any peptide, polypeptide or protein whose sequence is selected in such a way that the product of the fusion of this sequence has a sequence different from the wild-type sequence flanking the peptide to which it is fused.

As used herein, the term “composition” or “pharmaceutical composition” refers to a mixture of at least one compound useful with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration. The term “composition” is intended to encompass a product containing the specified ingredients (e.g., an antibody provided herein) in, optionally, the specified amounts.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans. The term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound provided herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound provided herein and are physiologically acceptable to the patient. Supplementary active compounds may also be incorporated into the compositions. The “pharmaceutically acceptable carrier” may further include a pharmaceutically acceptable salt of the compound provided herein. Other additional ingredients that may be included in the pharmaceutical compositions used, for example, in the practice methods provided herein are known in the art and described, for example in Remington's Pharmaceutical Sciences (Genaro, Ed., Mack Publishing Co., 1985, Easton, PA), which is incorporated herein by reference.

As used herein, “syndecan-2,” “Sdc2,” “Sdc-2,” “SDC-2” and like terms are interchangeable and refer to the protein encoded by the syndecan-2 gene (Sdc2). In some embodiments, the human Sdc2 and mouse Sdc2 proteins comprise the following amino acid sequences, respectively:

Human
(SEQ ID NO: 25)
MRRAWILLTLGLVACVSAESRAELTSDKDMYLDNSSIEEASGVYP
IDDDDYASASGSGADEDVESPELTTSRPLPKILLTSAAPKVETTT
LNIQNKIPAQTKSPEETDKEKVHLSDSERKMDPAEEDTNVYTEKH
SDSLFKRTEVLAAVIAGGVIGFLFAIFLILLLVYRMRKKDEGSYD
LGERKPSSAAYQKAPTKEFYA
Mouse
(SEQ ID NO: 26)
MQRAWILLTLGLMACVSAETRTELTSDKDMYLDNSSIEEASGVYP
IDDDDYSSASGSGADEDIESPVLTTSQLIPRIPLTSAASPKVETM
TLKTQSITPAQTESPEETDKEEVDISEAEEKLGPAIKSTDVYTEK
HSDNLFKRTEVLAAVIAGGVIGFLFAIFLILLLVYRMRKKDEGSY
DLGERKPSSAAYQKAPTKEFYA

As used herein, the terms “syndecan-2 extracellular domain” or “Sdc-2 ECD” refers to a peptide having the sequence of the extracellular domain of syndecan-2 and including its associated heparan sulfate chains, either isolated or linked to a heterologous peptide. In certain embodiments, the extracellular domain of syndecan-2 can be from human, mouse, or porcine syndecan-2 protein. As a non-limiting example, the amino acid sequence of the extracellular domain of human syndecan-2 is:

(SEQ ID NO: 27)
MYLDNSSIEE ASGVYPIDDD DYASASGSGA DEDVESPELT
TSRPLPKILL TSAAPKVETT TLNIQNKIPA QTKSPEETDK
EKVHLSDSER KMDPAEEDTN VYTEKHSDSL FKRTEVLAAV
IAGGVIGFLF AIFLILL

As used herein, “Density-Enhanced Phosphatase-1” or “Dep-1” refers to any native Dep-1 from any vertebrate source, including mammals such as primates (e.g., humans) and rodents (e.g., mice and rats), unless otherwise indicated. The term encompasses unprocessed Dep-1 as well as any form of Dep-1 that results from processing in the cell. The term also encompasses naturally occurring variants of Dep-1, e.g., splice variants or allelic variants. The amino acid sequence of an exemplary human Dep-1 is:

(SEQ ID NO: 439)
MKPAAREARLPPRSPGLRWALPLLLLLLRLGQILCAGGTPSPIPD
PSVATVATGENGITQISSTAESFHKQNGTGTPQVETNTSEDGESS
GANDSLRTPEQGSNGTDGASQKTPSSTGPSPVFDIKAVSISPTNV
ILTWKSNDTAASEYKYVVKHKMENEKTITVVHQPWCNITGLRPAT
SYVFSITPGIGNETWGDPRVIKVITEPIPVSDLRVALTGVRKAAL
SWSNGNGTASCRVLLESIGSHEELTQDSRLQVNISGLKPGVQYNI
NPYLLQSNKTKGDPLGTEGGLDASNTERSRAGSPTAPVHDESLVG
PVDPSSGQQSRDTEVLLVGLEPGTRYNATVYSQAANGTEGQPQAI
EFRTNAIQVFDVTAVNISATSLTLIWKVSDNESSSNYTYKIHVAG
ETDSSNLNVSEPRAVIPGLRSSTFYNITVCPVLGDIEGTPGFLQV
HTPPVPVSDFRVTVVSTTEIGLAWSSHDAESFQMHITQEGAGNSR
VEITTNQSIIIGGLFPGTKYCFEIVPKGPNGTEGASRTVCNRTVP
SAVFDIHVVYVTTTEMWLDWKSPDGASEYVYHLVIESKHGSNHTS
TYDKAITLOGLIPGTLYNITISPEVDHVWGDPNSTAQYTRPSNVS
NIDVSTNTTAATLSWQNFDDASPTYSYCLLIEKAGNSSNATQVVT
DIGITDATVTELIPGSSYTVEIFAQVGDGIKSLEPGRKSFCTDPA
SMASFDCEVVPKEPALVLKWTCPPGANAGFELEVSSGAWNNATHL
ESCSSENGTEYRTEVTYLNFSTSYNISITTVSCGKMAAPTRNTCT
TGITDPPPPDGSPNITSVSHNSVKVKFSGFEASHGPIKAYAVILT
TGEAGHPSADVLKYTYEDFKKGASDTYVTYLIRTEEKGRSQSLSE
VLKYEIDVGNESTTLGYYNGKLEPLGSYRACVAGFTNITFHPQNK
GLIDGAESYVSFSRYSDAVSLPQDPGVICGAVFGCIFGALVIVTV
GGFIFWRKKRKDAKNNEVSFSQIKPKKSKLIRVENFEAYFKKQQA
DSNCGFAEEYEDLKLVGISQPKYAAELAENRGKNRYNNVLPYDIS
RVKLSVQTHSTDDYINANYMPGYHSKKDFIATQGPLPNTLKDFWR
MVWEKNVYAIIMLTKCVEQGRTKCEEYWPSKQAQDYGDITVAMTS
EIVLPEWTIRDFTVKNIQTSESHPLRQFHFTSWPDHGVPDTTDLL
INFRYLVRDYMKQSPPESPILVHCSAGVGRTGTFIAIDRLIYQIE
NENTVDVYGIVYDLRMHRPLMVQTEDQYVFLNQCVLDIVRSQKDS
KVDLIYQNTTAMTIYENLAPVTTFGKINGYIA

The term “binding protein” refers to a protein comprising a portion (e.g., one or more binding regions such as CDRs) that binds to Sdc2, including human and/or cyno Sdc2 and, optionally, a scaffold or framework portion (e.g., one or more scaffold or framework regions) that allows the binding portion to adopt a conformation that promotes binding of the binding protein to a Sdc2 polypeptide, fragment, or epitope. Examples of such binding proteins include antibodies, such as a human antibody, a humanized antibody, a chimeric antibody, a recombinant antibody, a single chain antibody, a diabody, a triabody, a tetrabody, a Fab fragment, a F(ab′)₂ fragment, an IgD antibody, an IgE antibody, an IgM antibody, an IgG1 antibody, an IgG2 antibody, an IgG3 antibody, or an IgG4 antibody, and fragments thereof. The binding protein can comprise, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the binding protein as well as wholly synthetic scaffolds comprising, for example, a biocompatible polymer. See, e.g., Korndorfer et al., 2003, Proteins: Structure, Function, and Bioinformatics 53(1):121-29; and Roque et al., 2004, Biotechnol. Prog. 20:639-54. In addition, peptide antibody mimetics (“PAMs”) can be used, as well as scaffolds based on antibody mimetics utilizing fibronectin components as a scaffold. In the context of the present disclosure, a binding protein is said to specifically bind or selectively bind to Sdc2, for example, when the dissociation constant (K_{D}) is ≤10⁻⁷ M. In some embodiments, the binding proteins (e.g., antibodies) may specifically bind to Sdc2 with a K_{D} of from about 10⁻⁷ M to about 10⁻¹² M. In certain embodiments, the binding protein (e.g., antibody) may specifically bind to Sdc2 with high affinity when the K_{D} is ≤10⁻⁸ M or K_{D} is ≤10⁻⁹ M. In one embodiment, the binding proteins (e.g., antibodies) may specifically bind to purified human Sdc2 with a K_{D} of from 1×10⁻⁹ M to 10×10⁻⁹ M as measured by Biacore®. In another embodiment, the binding proteins (e.g., antibodies) may specifically bind to purified human Sdc2 with a K_{D} of from 0.1×10⁻⁹ M to 1×10⁻⁹ M as measured by KinExA™ (Sapidyne, Boise, ID). In yet another embodiment, the binding proteins (e.g., antibodies) specifically bind to human Sdc2 expressed on cells with a K_{D} of from 0.1×10⁻⁹ M to 10×10⁻⁹ M. In certain embodiments, the binding proteins (e.g., antibodies) specifically bind to human Sdc2 expressed on cells with a K_{D} of from 0.1×10⁻⁹ M to 1×10⁻⁹ M. In some embodiments, the binding proteins (e.g., antibodies) specifically bind to human Sdc2 expressed on cells with a K_{D} of 1×10⁻⁹ M to 10×10⁻⁹ M. In certain embodiments, the binding proteins (e.g., antibodies) specifically bind to human Sdc2 expressed on cells with a K_{D} of about 0.1×10⁻⁹ M, about 0.5×10⁻⁹ M, about 1×10⁻⁹ M, about 5×10⁻⁹ M, about 10×10⁻⁹ M, or any range or interval thereof. In still another embodiment, the binding proteins (e.g., antibodies) may specifically bind to cyno Sdc2 expressed on cells with a K_{D} of 0.1×10⁻⁹ M to 10×10⁻⁹ M. In certain embodiments, the binding proteins (e.g., antibodies) specifically bind to cyno Sdc2 expressed on cells with a K_{D} of from 0.1×10⁻⁹ M to 1×10⁻⁹ M. In some embodiments, the binding proteins (e.g., antibodies) specifically bind to cyno Sdc2 expressed on cells with a K_{D} of 1×10⁻⁹ M to 10×10⁻⁹ M. In certain embodiments, the binding proteins (e.g., antibodies) specifically bind to cyno Sdc2 expressed on cells with a K_{D} of about 0.1×10⁻⁹ M, about 0.5×10⁻⁹ M, about 1×10⁻⁹ M, about 5×10⁻⁹ M, about 10×10⁻⁹ M, or any range or interval thereof.

An “antigen” is a predetermined antigen to which an antibody can selectively bind. A target antigen may be a polypeptide, carbohydrate, nucleic acid, lipid, hapten, or other naturally occurring or synthetic compound. In some embodiments, the target antigen is a polypeptide.

The terms “antigen-binding fragment,” “antigen-binding domain,” “antigen-binding region,” and similar terms refer to that portion of an antibody, which comprises the amino acid residues that interact with an antigen and confer on the binding agent its specificity and affinity for the antigen (e.g., the CDRs).

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as Sdc2, is the affinity of the antibody or functional fragment for that epitope. The ratio of dissociation rate (k_{off}) to association rate (kₒₙ) of an antibody to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent Sdc2, come in contact with antibodies containing multiple binding sites, the interaction of antibody with antigen at one site will increase the probability of a reaction at a second site. The strength of such multiple interactions between a multivalent antibody and antigen is called the avidity. The avidity of an antibody can be a better measure of its binding capacity than is the affinity of its individual binding sites. For example, high avidity can compensate for low affinity as is sometimes found for pentameric IgM antibodies, which can have a lower affinity than IgG, but the high avidity of IgM, resulting from its multivalence, enables it to bind antigen effectively.

The term “compete” when used in the context of anti-Sdc2 antibodies (e.g., antibodies and binding proteins that bind to Sdc2 and compete for the same epitope or binding site on a target) means competition as determined by an assay in which the antibody (or binding fragment) thereof under study prevents or inhibits the specific binding of a reference molecule (e.g., a reference ligand or reference antigen-binding protein, such as a reference antibody) to a common antigen (e.g., Sdc2 or a fragment thereof). Numerous types of competitive binding assays can be used to determine if a test antibody competes with a reference antibody for binding to Sdc2 (e.g., human Sdc2). Examples of assays that can be employed include solid phase direct or indirect RIA, solid phase direct or indirect enzyme immunoassay (EIA), sandwich competition assay (see, e.g., Stahli et al., 1983, Methods in Enzymology 9:242-53), solid phase direct biotin-avidin EIA (see, e.g., Kirkland et al., 1986, J. Immunol. 137:3614-19), solid phase direct labeled assay, solid phase direct labeled sandwich assay (see, e.g., Harlow and Lane, Antibodies, A Laboratory Manual (1988)), solid phase direct label RIA using I-125 label (see, e.g., Morel et al., 1988, Mol. Immunol. 25:7-15), and direct labeled RIA (Moldenhauer et al., 1990, Scand. J. Immunol. 32:77-82). Typically, such an assay involves the use of a purified antigen (e.g., Sdc2 such as human Sdc2) bound to a solid surface, or cells bearing either of an unlabeled test antigen-binding protein (e.g., test anti-Sdc2 antibody) or a labeled reference antigen-binding protein (e.g., reference anti-Sdc2 antibody). Competitive inhibition may be measured by determining the amount of label bound to the solid surface or cells in the presence of the test antigen-binding protein. Usually the test antigen-binding protein is present in excess. Antibodies identified by competition assay (competing antibodies) include antibodies binding to the same epitope as the reference antibody and/or antibodies binding to an adjacent epitope sufficiently proximal to the epitope bound by the reference for antibodies steric hindrance to occur. Additional details regarding methods for determining competitive binding are described herein. Usually, when a competing antibody protein is present in excess, it will inhibit specific binding of a reference antibody to a common antigen by at least 30%, for example 40%, 45%, 50%, 55%, 60%, 65%, 70%, or 75%. In some instance, binding is inhibited by at least 80%, 85%, 90%, 95%, 96%, 97%, 98%, 99%, or more.

As used herein, the term an “isolated antibody” refers to an antibody which is substantially free of other antibodies having different antigenic specificities (e.g., an isolated antibody that specifically binds to Sdc2 is substantially free of antibodies that do not bind to Sdc2). In addition, an isolated antibody is substantially free of cellular material or other contaminating proteins from the cell or tissue source and/or other contaminant components from which the antibody is derived, or substantially free of chemical precursors or other chemicals when chemically synthesized. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 25%, 20%, 15%, 10%, 5%, or 1% (by dry weight) of heterologous protein (also referred to herein as a “contaminating protein”). In certain embodiments, when the antibody is recombinantly produced, it is substantially free of culture medium, e.g., culture medium represents less than about 20%, 15%, 10%, 5%, or 1% of the volume of the protein preparation. In certain embodiments, when the antibody is produced by chemical synthesis, it is substantially free of chemical precursors or other chemicals, for example, it is separated from chemical precursors or other chemicals that are involved in the synthesis of the protein. Accordingly, such preparations of the antibody have less than about 30%, 25%, 20%, 15%, 10%, 5%, or 1% (by dry weight) of chemical precursors or compounds other than the antibody of interest. Contaminant components can also include, but are not limited to, materials that would interfere with therapeutic uses for the antibody, and may include enzymes, hormones, and other proteinaceous or nonproteinaceous solutes. In certain embodiments, the antibody will be purified (1) to greater than 95% by weight of antibody as determined by the Lowry method (Lowry et al., 1951, J. Bio. Chem. 193:265-75), such as 96%, 97%, 98%, or 99%, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or silver stain. Isolated antibody includes the antibody in situ within recombinant cells since at least one component of the antibody's natural environment will not be present. Ordinarily, however, isolated antibody will be prepared by at least one purification step. In specific embodiments, antibodies provided herein are isolated.

The term “fusion protein” as used herein refers to a polypeptide that comprises an amino acid sequence of an antibody and an amino acid sequence of a heterologous polypeptide or protein (e.g., a polypeptide or protein not normally a part of the antibody (e.g., a non-anti-Sdc2 antigen-binding antibody)). The term “fusion” when used in relation to Sdc2 or to an anti-Sdc2 antibody refers to the joining of a peptide or polypeptide, or fragment, variant, and/or derivative thereof, with a heterologous peptide or polypeptide. In certain embodiments, the fusion protein retains the biological activity of the Sdc2 or anti-Sdc2 antibody. In certain embodiments, the fusion protein comprises a Sdc2 antibody VH region, VL region, VH CDR (one, two, or three VH CDRs), and/or VL CDR (one, two, or three VL CDRs), wherein the fusion protein binds to a Sdc2 epitope, a Sdc2 fragment, and/or a Sdc2 polypeptide.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (α), delta (δ), epsilon (ε), gamma (γ), and mu (μ), based on the amino acid sequence of the heavy chain constant region. The distinct heavy chains differ in size: α, δ, and γ contain approximately 450 amino acids, while u and & contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4. A heavy chain can be a human heavy chain.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (κ) or lambda (λ) based on the amino acid sequence of the constant domains. Light chain amino acid sequences are well known in the art. A light chain can be a human light chain.

The term “host” as used herein refers to an animal, such as a mammal (e.g., a human).

As used herein, the term “host cell” refers to a cell comprising a nucleic acid molecule provided herein. The “host cell” can be any type of cell, e.g., a primary cell, a cell in culture, or a cell from a cell line. In one embodiment, a “host cell” is a cell transfected with a nucleic acid molecule provided herein. In another embodiment, a “host cell” is a progeny or potential progeny of such a transfected cell. A progeny of a cell may or may not be identical to the parent cell, e.g., due to mutations or environmental influences that can occur in succeeding generations or integration of the nucleic acid molecule into the host cell genome. The term “host cell” as used herein refers to a particular subject cell that may be transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed antibody can be within the cytoplasm of a host cell, into the extracellular milieu such as the growth medium of a cell culture or anchored to the cell membrane.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The term “immune cell” is recognized in the art, as used herein refers to any cell involved in a host defense mechanism, such as cells that produces pro-inflammatory cytokines, and cells that participate in tissue damage and/or disease pathogenesis. Examples of immune cells include, but are not limited to, T cells, B cells, natural killer cells, neutrophils, mast cells, macrophages, antigen-presenting cells (APC), basophils, and eosinophils.

The term “monoclonal antibody” as used herein refers to an antibody obtained from a population of substantially homogeneous antibodies, e.g., the individual antibodies comprising the population are identical except for possible naturally occurring mutations that may be present in minor amounts, and each monoclonal antibody will typically recognize a single epitope on the antigen. In specific embodiments, a “monoclonal antibody,” as used herein, is an antibody produced by a single hybridoma or other cell, wherein the antibody binds to only a Sdc2 epitope as determined, for example, by ELISA or other antigen-binding or competitive binding assay known in the art. The term “monoclonal” is not limited to any particular method for making the antibody. For example, the monoclonal antibodies useful in the present disclosure may be prepared by the hybridoma methodology first described by Kohler et al., 1975, Nature 256:495, or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., 1991, Nature 352:624-28 and Marks et al., 1991, J. Mol. Biol. 222:581-97, for example. Other methods for the preparation of clonal cell lines and of monoclonal antibodies expressed thereby are well known in the art. See, e.g., Short Protocols in Molecular Biology (Ausubel et al. eds., 5th ed. 2002). Exemplary methods of producing monoclonal antibodies are provided in the Examples herein.

The term “native” when used in connection with biological materials such as nucleic acid molecules, polypeptides, host cells, and the like, refers to those which are found in nature and not manipulated, modified, and/or changed (e.g., isolated, purified, selected) by a human being.

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide. This definition of a human antibody specifically excludes a humanized antibody comprising non-human antigen-binding residues. Human antibodies can be produced using various techniques known in the art, including phage-display libraries (Hoogenboom and Winter, 1991, J. Mol. Biol. 227:381; Marks et al., 1991, J. Mol. Biol. 222:581) and yeast display libraries (Chao et al., 2006, Nature Protocols 1:755-68). Also available for the preparation of human monoclonal antibodies are methods described in Cole et al., Monoclonal Antibodies and Cancer Therapy 77 (1985); Boerner et al., 1991, J. Immunol. 147(1):86-95; and van Dijk and van de Winkel, 2001, Curr. Opin. Pharmacol. 5:368-74. Human antibodies can be prepared by administering the antigen to a transgenic animal that has been modified to produce such antibodies in response to antigenic challenge, but whose endogenous loci have been disabled, e.g., mice (see, e.g., Jakobovits, 1995, Curr. Opin. Biotechnol. 6(5):561-66; Brüggemann and Taussing, 1997, Curr. Opin. Biotechnol. 8(4):455-58; and U.S. Pat. Nos. 6,075,181 and 6,150,584 regarding XENOMOUSE™ technology). See also, for example, Li et al., 2006, Proc. Natl. Acad. Sci. USA 103:3557-62 regarding human antibodies generated via a human B-cell hybridoma technology.

“Fully human” refers to an immunoglobulin, such as an antibody or antigen-binding fragment thereof (such as Fv, Fab, Fab′, F(ab′)₂ or other antigen-binding subsequences of antibodies), where the whole molecule is of human origin or consists of an amino acid sequence identical to a human form of the antibody.

As used herein, the terms “humanized” and “chimeric” antibodies or antigen-binding fragments thereof (such as Fv, Fab, Fab′, F(ab′)₂ or other antigen-binding subsequences of antibodies) refer to immunoglobulins which contain minimal sequences derived from non-human sources. For the most part, humanized and chimeric immunoglobulins are human-origin immunoglobulins in which complementary-determining region (CDR) residues are replaced by those from a CDR of a non-human species such as mouse, rat or rabbit having the desired specificity, affinity, and capacity. In some instances, Fv framework region (FR) residues of the human immunoglobulin are replaced by corresponding non-human residues. Furthermore, humanized and chimeric antibodies or antigen-binding fragments thereof can comprise residues which are found neither in the recipient human antibody nor in the imported CDR or framework sequences. These modifications are made to further refine and optimize antibody performance. In general, the humanized and chimeric antibody or antigen-binding fragment thereof will comprise substantially all of at least one, and typically two, variable domains, in which all or substantially all of the CDR regions correspond to those of a non-human immunoglobulin and all or substantially all of the FR regions are those of a human immunoglobulin sequence. The World Health Organization (WHO) International Nonproprietary Name (INN) Expert Group has defined requirements for non-human derived antibodies to be considered “humanized”. According to guidelines, comparison of a candidate antibody to human sequences should be done through the International Immunogenetics Information System® (IMGT®) DomainGapAlign tool (imgt.org). This tool interrogates the IMGT® database of antibody germline variable region genes where the alignment score is made only against germline sequence variable region exons, thus omitting part of CDR3 and the J region from the analysis. For an antibody to be “humanized”, in addition to being “closer to human than to other species”, the top “hit” should be human and the identity to human sequences must be at least 85%, otherwise the antibody would be designated as “chimeric”. For further details, see Jones et al., Nature, 321: 522-525, 1986; Reichmann et al., Nature, 332: 323-329, 1988; Presta, Curr. Op. Struct. Biol., 2: 593-596, 1992.

As used herein, the term “chimeric antibody” refers to an antibody wherein the amino acid sequence of the immunoglobulin molecule is derived from two or more species. The variable region of both the light and heavy chains often correspond to the variable region of an antibody derived from one species of mammal (e.g., mouse, rat, rabbit, etc.) having the desired specificity, affinity, and capability, while the constant regions correspond to the sequences of an antibody derived from another species of mammal (e.g., human) to avoid eliciting an immune response in that species. In some embodiments, the antibodies comprise a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., 1984, Proc. Natl. Acad. Sci. USA 81:6851-55).

As used herein, the term “humanized antibody” refers to a non-human antibody that is modified to increase the sequence homology to that of a human antibody, such that the antigen-binding properties of the antibody are retained, but its antigenicity in the human body is reduced. “Humanized” forms of nonhuman (e.g., murine) antibodies are chimeric antibodies that include human immunoglobulins (e.g., recipient antibody) in which the native CDR residues are replaced by residues from the corresponding CDR of a nonhuman species (e.g., donor antibody) such as mouse, rat, rabbit, or nonhuman primate having the desired specificity, affinity, and capacity. In some instances, one or more FR region residues of the human immunoglobulin are replaced by corresponding nonhuman residues. Furthermore, humanized antibodies can comprise residues that are not found in the recipient antibody or in the donor antibody. These modifications are made to further refine antibody performance. A humanized antibody heavy or light chain can comprise substantially all of at least one or more variable regions, in which all or substantially all of the CDRs correspond to those of a nonhuman immunoglobulin and all or substantially all of the FRs are those of a human immunoglobulin sequence. In certain embodiments, the humanized antibody will comprise at least a portion of an immunoglobulin constant region (Fc), typically that of a human immunoglobulin. For further details, see, Jones et al., 1986, Nature 321:522-25; Riechmann et al., 1988, Nature 332:323-29; Presta, 1992, Curr. Op. Struct. Biol. 2:593-96; Carter et al., 1992, Proc. Natl. Acad. Sci. USA 89:4285-89; U.S. Pat. Nos. 6,800,738; 6,719,971; 6,639,055; 6,407,213; and 6,054,297.

An “affinity matured” antibody is one with one or more alterations (e.g., amino acid sequence variations, including changes, additions, and/or deletions) in one or more HVRs thereof which result in an improvement in the affinity of the antibody for antigen, compared to a parent antibody which does not possess those alteration(s). Affinity matured antibodies can have nanomolar or even picomolar affinities for the target antigen. Affinity matured antibodies are produced by procedures known in the art. For review, see Hudson and Souriau, 2003, Nature Medicine 9:129-34; Hoogenboom, 2005, Nature Biotechnol. 23:1105-16; Quiroz and Sinclair, 2010, Revista Ingeneria Biomedia 4:39-51.

A “blocking” antibody or an “antagonist” antibody is one which inhibits or reduces biological activity of the antigen it binds. For example, blocking antibodies or antagonist antibodies may substantially or completely inhibit the biological activity of the antigen.

An “agonist” antibody is an antibody that triggers a response, e.g., one that mimics at least one of the functional activities of a polypeptide of interest. An agonist antibody includes an antibody that is a ligand mimetic, for example, wherein a ligand binds to a cell surface receptor and the binding induces cell signaling or activities via an intercellular cell signaling pathway and wherein the antibody induces a similar cell signaling or activation.

“Binding affinity” generally refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. Specific illustrative embodiments include the following. In one embodiment, the “K_{D}” or “K_{D} value” may be measured by assays known in the art, for example by a binding assay. The K_{D} may be measured in a RIA, for example, performed with the Fab version of an antibody of interest and its antigen (Chen et al., 1999, J. Mol Biol 293:865-81). The K_{D} or K_{D} value may also be measured by using surface plasmon resonance assays by Biacore®, using, for example, a Biacore® TM-2000 or a Biacore® TM-3000, or by biolayer interferometry using, for example, the Octet® QK384 system. An “on-rate” or “rate of association” or “association rate” or “kₒₙ” may also be determined with the same surface plasmon resonance or biolayer interferometry techniques described above using, for example, a Biacore® TM-2000 or a Biacore® TM-3000, or the Octet® QK384 system.

The phrase “substantially similar” or “substantially the same” denotes a sufficiently high degree of similarity between two numeric values (e.g., one associated with an antibody of the present disclosure and the other associated with a reference antibody) such that one of skill in the art would consider the difference between the two values to be of little or no biological and/or statistical significance within the context of the biological characteristic measured by the values (e.g., K_{D} values). For example, the difference between the two values may be less than about 50%, less than about 40%, less than about 30%, less than about 20%, less than about 10%, or less than about 5%, as a function of the value for the reference antibody.

The phrase “substantially increased,” “substantially reduced,” or “substantially different,” as used herein, denotes a sufficiently high degree of difference between two numeric values (e.g., one associated with an antibody of the present disclosure and the other associated with a reference antibody) such that one of skill in the art would consider the difference between the two values to be of statistical significance within the context of the biological characteristic measured by the values. For example, the difference between said two values can be greater than about 10%, greater than about 20%, greater than about 30%, greater than about 40%, or greater than about 50%, as a function of the value for the reference antibody.

The term “inhibition” or “inhibit,” when used herein, refers to partial (such as, 1%, 2%, 5%, 10%, 20%, 25%, 50%, 75%, 90%, 95%, 99%) or complete (i.e., 100%) inhibition.

“Antibody effector functions” refer to the biological activities attributable to the Fc region (e.g., a native sequence Fc region or amino acid sequence variant Fc region) of an antibody and vary with the antibody isotype. Examples of antibody effector functions include but are not limited to: C1q binding; CDC; Fc receptor binding; ADCC; phagocytosis; downregulation of cell surface receptors (e.g., B cell receptor); and B cell activation.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays as disclosed.

A “native sequence Fc region” comprises an amino acid sequence identical to the amino acid sequence of an Fc region found in nature, and not manipulated, modified, and/or changed (e.g., isolated, purified, selected, including or combining with other sequences such as variable region sequences) by a human. Native sequence human IgG1 Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

The term “variant” when used in relation to Sdc2 or to an anti-Sdc2 antibody may refer to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence. For example, a Sdc2 variant may result from one or more (such as, for example, about 1 to about 25, about 1 to about 20, about 1 to about 15, about 1 to about 10, or about 1 to about 5) changes to an amino acid sequence of a native Sdc2. Also, by way of example, a variant of an anti-Sdc2 antibody may result from one or more (such as, for example, about 1 to about 25, about 1 to about 20, about 1 to about 15, about 1 to about 10, or about 1 to about 5) changes to an amino acid sequence of a native or previously unmodified anti-Sdc2 antibody. Variants may be naturally occurring, such as allelic or splice variants, or may be artificially constructed. Polypeptide variants may be prepared from the corresponding nucleic acid molecules encoding the variants. In specific embodiments, the Sdc2 variant or anti-Sdc2 antibody variant at least retains Sdc2 or anti-Sdc2 antibody functional activity, respectively. In specific embodiments, an anti-Sdc2 antibody variant binds Sdc2 and/or is antagonistic to Sdc2 activity. In specific embodiments, an anti-Sdc2 antibody variant binds Sdc2 and/or is agonistic to Sdc2 activity. In certain embodiments, the variant is encoded by a single nucleotide polymorphism (SNP) variant of a nucleic acid molecule that encodes Sdc2 or anti-Sdc2 antibody VH or VL regions or subregions, such as one or more CDRs.

As used herein, the term “vector” is a replicon in which another nucleic acid segment can be operably inserted so as to bring about the replication or expression of the segment.

The term “vector” refers to a substance that is used to carry or include a nucleic acid sequence, including for example, a nucleic acid sequence encoding an anti-Sdc2 antibody as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product (e.g., an anti-Sdc2 antibody as described herein), and it is further understood that expression levels can be optimized to obtain sufficient expression using methods well known in the art.

“Antibody-dependent cell-mediated cytotoxicity” or “ADCC” refers to a form of cytotoxicity in which secreted immunoglobulin bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. NK cells, the primary cells for mediating ADCC, express FcγRIII only, whereas monocytes express FcγRI, FcγRII, and FcγRIII. FcR expression on hematopoietic cells is known (see, e.g., Ravetch and Kinet, 1991, Annu. Rev. Immunol. 9:457-92). To assess ADCC activity of a molecule of interest, an in vitro ADCC assay (see, e.g., U.S. Pat. Nos. 5,500,362 and 5,821,337) can be performed. Useful effector cells for such assays include peripheral blood mononuclear cells (PBMC) and Natural Killer (NK) cells. Alternatively or additionally, ADCC activity of the molecule of interest may be assessed in vivo, for example, in an animal model (see, e.g., Clynes et al., 1998, Proc. Natl. Acad. Sci. USA 95:652-56). Antibodies with little or no ADCC activity may be selected for use.

“Antibody-dependent cellular phagocytosis” or “ADCP” refers to the destruction of target cells via monocyte or macrophage-mediated phagocytosis when immunoglobulin bound onto Fc receptors (FcRs) present on certain phagocytotic cells (e.g., neutrophils, monocytes, and macrophages) enable these phagocytotic cells to bind specifically to an antigen-bearing target cell and subsequently kill the target cell. To assess ADCP activity of a molecule of interest, an in vitro ADCP assay (see, e.g., Bracher et al., 2007, J. Immunol. Methods 323:160-71) can be performed. Useful phagocytotic cells for such assays include peripheral blood mononuclear cells (PBMC), purified monocytes from PBMC, or U937 cells differentiated to the mononuclear type. Alternatively or additionally, ADCP activity of the molecule of interest may be assessed in vivo, for example, in an animal model (see, e.g., Wallace et al., 2001, J. Immunol. Methods 248:167-82). Antibodies with little or no ADCP activity may be selected for use.

“Fc receptor” or “FcR” describes a receptor that binds to the Fc region of an antibody. An exemplary FcR is a native sequence human FcR. Moreover, an exemplary FcR is one that binds an IgG antibody (e.g., a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof (see, e.g., Daëron, 1997, Annu. Rev. Immunol. 15:203-34). Various FcRs are known (see, e.g., Ravetch and Kinet, 1991, Annu. Rev. Immunol. 9:457-92; Capel et al., 1994, Immunomethods 4:25-34; and de Haas et al., 1995, J. Lab. Clin. Med. 126:330-41). Other FcRs, including those to be identified in the future, are encompassed by the term “FcR” herein. The term also includes the neonatal receptor, FcRn, which is responsible for the transfer of maternal IgGs to the fetus (see, e.g., Guyer et al., 1976, J. Immunol. 117:587-93; and Kim et al., 1994, Eu. J. Immunol. 24:2429-34). Antibody variants with improved or diminished binding to FcRs have been described (see, e.g., WO 2000/42072; U.S. Pat. Nos. 7,183,387; 7,332,581; and 7,335,742; Shields et al. 2001, J. Biol. Chem. 9(2):6591-604).

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay (see, e.g., Gazzano-Santoro et al., 1996, J. Immunol. Methods 202:163) may be performed. Polypeptide variants with altered Fc region amino acid sequences (polypeptides with a variant Fc region) and increased or decreased C1q binding capability have been described (see, e.g., U.S. Pat. No. 6,194,551; WO 1999/51642; Idusogie et al., 2000, J. Immunol. 164:4178-84). Antibodies with little or no CDC activity may be selected for use.

A Sdc2 polypeptide “extracellular domain” or “ECD” refers to a form of the Sdc2 polypeptide that is essentially free of the transmembrane and cytoplasmic domains. For example, a Sdc2 polypeptide ECD may have less than 1% of such transmembrane and/or cytoplasmic domains and can have less than 0.5% of such domains.

The term “identity” refers to a relationship between the sequences of two or more polypeptide molecules or two or more nucleic acid molecules, as determined by aligning and comparing the sequences. “Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALIGN (DNAStar, Inc.) software. Those skilled in the art can determine appropriate parameters for aligning sequences, including any algorithms needed to achieve maximal alignment over the full length of the sequences being compared.

The terms “identical” or percent “identity,” in the context of two or more polypeptide sequences (e.g., a Sdc2 antibody) or nucleic acid sequences thereof, refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215:403-410 and Altschul et al. (1997) Nucleic Acids Res. 25:3389-3402, respectively. Software for performing BLAST analyses is publicly available through the National Center for Biotechnology Information. This algorithm involves first identifying high scoring sequence pairs (HSPs) by identifying short words of length W in the query sequence, which cither match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

A “modification” of an amino acid residue/position refers to a change of a primary amino acid sequence as compared to a starting amino acid sequence, wherein the change results from a sequence alteration involving said amino acid residue/position. For example, typical modifications include substitution of the residue with another amino acid (e.g., a conservative or non-conservative substitution), insertion of one or more (e.g., generally fewer than 5, 4, or 3) amino acids adjacent to said residue/position, and/or deletion of said residue/position.

An “epitope” is the site on the surface of an antigen molecule to which a single antibody molecule binds, such as a localized region on the surface of an antigen, such as a Sdc2 polypeptide, a Sdc2 polypeptide fragment, that is capable of being bound to one or more antigen binding regions of an antibody, and that has antigenic or immunogenic activity in an animal, such as a mammal (e.g., a human), that is capable of eliciting an immune response. An epitope having immunogenic activity is a portion of a polypeptide that elicits an antibody response in an animal. An epitope having antigenic activity is a portion of a polypeptide to which an antibody binds as determined by any method well known in the art, including, for example, by an immunoassay. Antigenic epitopes need not necessarily be immunogenic. Epitopes often consist of chemically active surface groupings of molecules such as amino acids or sugar side chains and have specific three-dimensional structural characteristics as well as specific charge characteristics. Antibody epitopes may be linear epitopes or conformational epitopes. Linear epitopes are formed by a continuous sequence of amino acids in a protein. Conformational epitopes are formed of amino acids that are discontinuous in the protein sequence, but which are brought together upon folding of the protein into its three-dimensional structure. Induced epitopes are formed when the three-dimensional structure of the protein is in an altered conformation, such as following activation or binding of another protein or ligand. In certain embodiments, a Sdc2 epitope is a three-dimensional surface feature of a Sdc2 polypeptide. In other embodiments, a Sdc2 epitope is linear feature of a Sdc2 polypeptide. Generally, an antigen has several or many different epitopes and may react with many different antibodies.

An antibody binds “an epitope,” “essentially the same epitope,” or “the same epitope” as a reference antibody, when the two antibodies recognize identical, overlapping, or adjacent epitopes in a three-dimensional space. The most widely used and rapid methods for determining whether two antibodies bind to identical, overlapping, or adjacent epitopes in a three-dimensional space are competition assays, which can be configured in a number of different formats, for example, using either labeled antigen or labeled antibody. In some assays, the antigen is immobilized on a 96-well plate, or expressed on a cell surface, and the ability of unlabeled antibodies to block the binding of labeled antibodies is measured using radioactive, fluorescent, or enzyme labels.

“Epitope mapping” is the process of identifying the binding sites, or epitopes, of antibodies on their target antigens. “Epitope binning” is the process of grouping antibodies based on the epitopes they recognize. More particularly, epitope binning comprises methods and systems for discriminating the epitope recognition properties of different antibodies, using competition assays combined with computational processes for clustering antibodies based on their epitope recognition properties and identifying antibodies having distinct binding specificities.

An “effective amount” of a delivery vehicle is that amount sufficient to effectively bind or deliver a compound. An “effective amount” is generally an amount sufficient to reduce the severity and/or frequency of symptoms, eliminate the symptoms and/or underlying cause, prevent the occurrence of symptoms and/or their underlying cause, and/or improve or remediate the damage that results from or is associated with a disease, disorder, or condition, including, for example, Sdc2-associated diseases and disorders (e.g., stroke (e.g., ischemic stroke), inflammatory eye disorders (e.g., AMD), and cardiovascular disorders (e.g., myocardial infarction)). In some embodiments, the effective amount is a therapeutically effective amount or a prophylactically effective amount.

The term “therapeutically effective amount” as used herein refers to the amount of an agent (e.g., an antibody provided herein or any other agent described herein) that is sufficient to reduce and/or ameliorate the severity and/or duration of a given disease, disorder, or condition, and/or a symptom related thereto, for example, Sdc2-associated diseases and disorders (e.g., stroke (e.g., ischemic stroke), inflammatory eye disorders (e.g., AMD), and cardiovascular disorders (e.g., myocardial infarction)). A “therapeutically effective amount” of a substance/molecule/agent of the present disclosure (e.g., an anti-Sdc2 antibody) may vary according to factors such as the disease state, age, sex, and weight of the individual, and the ability of the substance/molecule/agent to elicit a desired response in the individual. A therapeutically effective amount encompasses an amount in which any toxic or detrimental effects of the substance/molecule/agent are outweighed by the therapeutically beneficial effects. In certain embodiments, the term “therapeutically effective amount” refers to an amount of an antibody or other agent (e.g., drug) effective to “treat” a disease, disorder, or condition, in a subject or mammal.

A “prophylactically effective amount” is an amount of a pharmaceutical composition that, when administered to a subject, will have the intended prophylactic effect, e.g., preventing, delaying, or reducing the likelihood of the onset (or reoccurrence) of a disease, disorder, condition, or associated symptom(s), for example, Sdc2-associated diseases and disorders (e.g., stroke (e.g., ischemic stroke), inflammatory eye disorders (e.g., AMD), and cardiovascular disorders (e.g., myocardial infarction)). Typically, but not necessarily, since a prophylactic dose is used in subjects prior to or at an earlier stage of a disease, disorder, or condition, a prophylactically effective amount may be less than a therapeutically effective amount. The full therapeutic or prophylactic effect does not necessarily occur by administration of one dose and may occur only after administration of a series of doses. Thus, a therapeutically or prophylactically effective amount may be administered in one or more administrations.

“Chronic” administration refers to administration of the agent(s) in a continuous mode (e.g., for a period of time such as days, weeks, months, or years) as opposed to an acute mode, so as to maintain the initial therapeutic effect (activity) for an extended period of time. “Intermittent” administration is treatment that is not consecutively done without interruption, but rather is cyclic in nature.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. The term “carrier” can also refer to a diluent, adjuvant (e.g., Freund's adjuvant (complete or incomplete)), excipient, or vehicle. Such carriers, including pharmaceutical carriers, can be sterile liquids, such as water and oils, including those of petroleum, animal, vegetable, or synthetic origin, such as peanut oil, soybean oil, mineral oil, sesame oil, and the like. Water is an exemplary carrier when a composition (e.g., a pharmaceutical composition) is administered intravenously. Saline solutions and aqueous dextrose and glycerol solutions can also be employed as liquid carriers, particularly for injectable solutions. Suitable excipients (e.g., pharmaceutical excipients) include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard carriers such as pharmaceutical grades of mannitol, lactose, starch, magnesium stearate, sodium saccharine, cellulose, magnesium carbonate, etc. Examples of suitable pharmaceutical carriers are described in Remington and Gennaro, Remington's Pharmaceutical Sciences (18th ed. 1990). Compositions, including pharmaceutical compounds, may contain an anti-Sdc2 antibody, for example, in isolated or purified form, together with a suitable amount of carriers.

“Polyclonal antibodies” as used herein refer to an antibody population generated in an immunogenic response to a protein having many epitopes and thus includes a variety of different antibodies directed to the same or different epitopes within the protein. Methods for producing polyclonal antibodies are known in the art (See, e.g., Short Protocols in Molecular Biology (Ausubel et al. eds., 5th ed. 2002)).

An “isolated nucleic acid” is a nucleic acid, for example, an RNA, DNA, or a mixed nucleic acid, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding an antibody as described herein are isolated or purified. The term embraces nucleic acid sequences that have been removed from their naturally occurring environment and includes recombinant or cloned DNA isolates and chemically synthesized analogues or analogues biologically synthesized by heterologous systems. A substantially pure molecule may include isolated forms of the molecule.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides. “Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces an anti-Sdc2 antibody of the present disclosure may include a parent hybridoma cell, as well as bacterial and eukaryotic host cells into which nucleic acids encoding the antibodies have been introduced. Suitable host cells are disclosed below.

Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

The terms “prevent,” “preventing,” and “prevention” refer to reducing the likelihood of the onset (or recurrence) of a disease, disorder, condition, or associated symptom(s) (e.g., AMD).

The term “prophylactic agent” refers to any agent that can totally or partially inhibit the development, recurrence, onset, or spread of a disease, for example, Sdc2-associated diseases and disorders (e.g., stroke (e.g., ischemic stroke), inflammatory eye disorders (e.g., AMD), and cardiovascular disorders (e.g., myocardial infarction)) and/or symptom related thereto in a subject. In certain embodiments, the term “prophylactic agent” refers to an anti-Sdc2 antibody as described herein.

As used herein, a “prophylactically effective serum titer” is the serum titer of a Sdc2 antibody, e.g., a Sdc2 antibody as described herein, in a subject (e.g., a human), that totally or partially inhibits the development, recurrence, onset, or spread of a disease, disorder, or condition, for example, Sdc2-associated diseases and disorders (e.g., stroke (e.g., ischemic stroke), inflammatory eye disorders (e.g., AMD), and cardiovascular disorders (e.g., myocardial infarction)) and/or symptom related thereto in the subject.

In certain embodiments, a “therapeutically effective serum titer” is the serum titer of a Sdc2 antibody, e.g., a Sdc2 antibody as described herein, in a subject (e.g., a human), that reduces the severity, the duration, and/or the symptoms associated with a Sdc2-mediated disease, disorder, or condition, in the subject.

The term “recombinant antibody” refers to an antibody that is prepared, expressed, created, or isolated by recombinant means. Recombinant antibodies can be antibodies expressed using a recombinant expression vector transfected into a host cell, antibodies isolated from a recombinant, combinatorial antibody library, antibodies isolated from an animal (e.g., a mouse or cow) that is transgenic and/or transchromosomal for human immunoglobulin genes (see, e.g., Taylor et al., 1992, Nucl. Acids Res. 20:6287-95), or antibodies prepared, expressed, created, or isolated by any other means that involves splicing of immunoglobulin gene sequences to other DNA sequences. Such recombinant antibodies can have variable and constant regions, including those derived from human germline immunoglobulin sequences (See Kabat et al., supra). In certain embodiments, however, such recombinant antibodies may be subjected to in vitro mutagenesis (or, when an animal transgenic for human Ig sequences is used, in vivo somatic mutagenesis), thus the amino acid sequences of the VH and VL regions of the recombinant antibodies are sequences that, while derived from and related to human germline VH and VL sequences, may not naturally exist within the human antibody germline repertoire in vivo.

The term “serum titer” refers to an average serum titer in a subject from multiple samples (e.g., at multiple time points) or in a population of at least 10, at least 20, at least 40 subjects, up to about 100, 1000, or more.

The term “side effects” encompasses unwanted and/or adverse effects of a therapy (e.g., a prophylactic or therapeutic agent). Unwanted effects are not necessarily adverse. An adverse effect from a therapy (e.g., a prophylactic or therapeutic agent) might be harmful, uncomfortable, or risky. Examples of side effects include, diarrhea, cough, gastroenteritis, wheezing, nausea, vomiting, anorexia, abdominal cramping, fever, pain, loss of body weight, dehydration, alopecia, dyspnea, insomnia, dizziness, mucositis, nerve and muscle effects, fatigue, dry mouth, loss of appetite, rashes or swellings at the site of administration, flu-like symptoms such as fever, chills, and fatigue, digestive tract problems, and allergic reactions. Additional undesired effects experienced by patients are numerous and known in the art. Many are described in Physician's Desk Reference (68th ed. 2014).

The terms “subject,” “patient,” “individual and the like are used interchangeably. As used herein, in certain embodiments, a subject is a mammal, such as a non-primate (e.g., cow, pig, horse, cat, dog, rat, etc.) or a primate (e.g., monkey and human). In specific embodiments, the subject is a human. In one embodiment, the subject is a mammal (e.g., a human) having a disease, disorder, or condition. In another embodiment, the subject is a mammal (e.g., a human) at risk of developing a disease, disorder, or condition, for example, Sdc2-associated diseases and disorders (e.g., stroke (e.g., ischemic stroke), inflammatory eye disorders (e.g., AMD), and cardiovascular disorders (e.g., myocardial infarction)).

“Substantially all” refers to at least about 60%, at least about 65%, at least about 70%, at least about 75%, at least about 80%, at least about 85%, at least about 90%, at least about 95%, at least about 98%, at least about 99%, or about 100%.

The term “therapeutic agent” refers to any agent that can be used in treating, preventing, or alleviating a disease, disorder, or condition, including in the treatment, prevention, or alleviation of one or more symptoms of a disease, disorder, or condition, for example, Sdc2-associated diseases and disorders (e.g., stroke (e.g., ischemic stroke), inflammatory eye disorders (e.g., AMD), and cardiovascular disorders (e.g., myocardial infarction)) and/or a symptom related thereto. In certain embodiments, a therapeutic agent refers to an anti-Sdc2 antibody as described herein.

The term “therapy” refers to any protocol, method, and/or agent that can be used in the prevention, management, treatment, and/or amelioration of a disease, disorder, or, for example, Sdc2-associated diseases and disorders (e.g., stroke (e.g., ischemic stroke), inflammatory eye disorders (e.g., AMD), and cardiovascular disorders (e.g., myocardial infarction)). In certain embodiments, the terms “therapies” and “therapy” refer to a biological therapy, supportive therapy, and/or other therapies useful in the prevention, management, treatment, and/or amelioration of a disease, disorder, or condition, for example, Sdc2-associated diseases and disorders (e.g., stroke (e.g., ischemic stroke), inflammatory eye disorders (e.g., AMD), and cardiovascular disorders (e.g., myocardial infarction)), known to one of skill in the art such as medical personnel.

The term “detectable probe” refers to a composition that provides a detectable signal. The term includes, without limitation, any fluorophore, chromophore, radiolabel, enzyme, antibody or antibody fragment, and the like, that provide a detectable signal via its activity.

The term “detectable agent” refers to a substance that can be used to ascertain the existence or presence of a desired molecule, such as an anti-Sdc2 antibody as described herein, in a sample or subject. A detectable agent can be a substance that is capable of being visualized or a substance that is otherwise able to be determined and/or measured (e.g., by quantitation).

The term “diagnostic agent” refers to a substance administered to a subject that aids in the diagnosis of a disease, disorder, or condition. Such substances can be used to reveal, pinpoint, and/or define the localization of a disease-causing process. In certain embodiments, a diagnostic agent includes a substance that is conjugated to an anti-Sdc2 antibody as described herein, that when administered to a subject or contacted with a sample from a subject, aids in the diagnosis of a disease, for example, Sdc2-associated diseases and disorders (e.g., stroke (e.g., ischemic stroke), inflammatory eye disorders (e.g., AMD), and cardiovascular disorders (e.g., myocardial infarction)).

The term “encoding nucleic acid” or grammatical equivalents thereof as it is used in reference to nucleic acid molecule refers to a nucleic acid molecule in its native state or when manipulated by methods well known to those skilled in the art that can be transcribed to produce mRNA, which is then translated into a polypeptide and/or a fragment thereof. The antisense strand is the complement of such a nucleic acid molecule, and the encoding sequence can be deduced therefrom.

The term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.), and polyols (e.g., mannitol, sorbitol, etc.). See, also, Remington and Gennaro, Remington's Pharmaceutical Sciences (18th ed. 1990), which is hereby incorporated by reference in its entirety.

In the context of a peptide or polypeptide, the term “fragment” as used herein refers to a peptide or polypeptide that comprises less than the full-length amino acid sequence. Such a fragment may arise, for example, from a truncation at the amino terminus, a truncation at the carboxy terminus, and/or an internal deletion of a residue(s) from the amino acid sequence. Fragments may, for example, result from alternative RNA splicing or from in vivo protease activity. In certain embodiments, Sdc2 fragments or anti-Sdc2 antibody fragments include polypeptides comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 30 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least contiguous 100 amino acid residues, at least 125 contiguous amino acid residues, at least 150 contiguous amino acid residues, at least 175 contiguous amino acid residues, at least 200 contiguous amino acid residues, at least 250, at least 300, at least 350, at least 400, at least 450, at least 500, at least 550, at least 600, at least 650, at least 700, at least 750, at least 800, at least 850, at least 900, or at least 950 contiguous amino acid residues of the amino acid sequence of a Sdc2 polypeptide or an anti-Sdc2 antibody. In a specific embodiment, a fragment of a Sdc2 polypeptide or an anti-Sdc2 antibody retains at least 1, at least 2, at least 3, or more functions of the polypeptide or antibody.

As used herein, the terms “treat,” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and curing the disease. As used herein, “treating a disease or disorder” means reducing the frequency with which a symptom of the disease or disorder is experienced by a patient. Disease and disorder are used interchangeably herein. As used herein, the term “treatment” or “treating” encompasses prophylaxis and/or therapy. Accordingly, the compositions and methods provided herein are not limited to therapeutic applications and can be used in prophylactic ones. Therefore “treating” or “treatment” of a state, disorder or condition includes: (i) preventing or delaying the appearance of clinical symptoms of the state, disorder or condition developing in a subject that may be afflicted with or predisposed to the state, disorder or condition but docs not yet experience or display clinical or subclinical symptoms of the state, disorder or condition, (ii) inhibiting the state, disorder or condition, i.e., arresting or reducing the development of the disease or at least one clinical or subclinical symptom thereof, or (iii) relieving the disease, i.e. causing regression of the state, disorder or condition or at least one of its clinical or subclinical symptoms. As used herein, the terms “treat,” “treating,” and “treatment” are all intended to refer to an amelioration or reversal of at least one measurable physical parameter related to a disease or disorder, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or more preferably a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

The terms “manage,” “managing,” and “management” refer to the beneficial effects that a subject derives from a therapy (e.g., a prophylactic or therapeutic agent), which does not result in a cure of the disease. In certain embodiments, a subject is administered one or more therapies (e.g., prophylactic or therapeutic agents, such as an antibody provided herein) to “manage” a disease, for example, Sdc2-associated diseases and disorders (e.g., stroke (e.g., ischemic stroke), inflammatory eye disorders (e.g., AMD), and cardiovascular disorders (e.g., myocardial infarction)), one or more symptoms thereof, so as to prevent the progression or worsening of the disease.

“Administer” or “administration” refers to the act of injecting or otherwise physically delivering a substance as it exists outside the body (e.g., an anti-Sdc2 antibody as described herein) into a patient, such as by mucosal, intradermal, intravenous, intramuscular delivery, and/or any other method of physical delivery described herein or known in the art. When a disease, disorder, condition, or a symptom thereof, is being treated, administration of the substance typically occurs after the onset of the disease, disorder, condition, or symptoms thereof. When a disease, disorder, condition, or symptoms thereof, are being prevented, administration of the substance typically occurs before the onset of the disease, disorder, condition, or symptoms thereof.

In the context of a polypeptide, the term “analog” as used herein refers to a polypeptide that possesses a similar or identical function as a Sdc2 polypeptide, a fragment of a Sdc2 polypeptide, or an anti-Sdc2 antibody but does not necessarily comprise a similar or identical amino acid sequence of a Sdc2 polypeptide, a fragment of a Sdc2 polypeptide, or an anti-Sdc2 antibody, or possess a similar or identical structure of a Sdc2 polypeptide, a fragment of a Sdc2 polypeptide, or an anti-Sdc2 antibody. A polypeptide that has a similar amino acid sequence refers to a polypeptide that satisfies at least one of the followings: (a) a polypeptide having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of a Sdc2 polypeptide, a fragment of a Sdc2 polypeptide, or an anti-Sdc2 antibody provided herein; (b) a polypeptide encoded by a nucleotide sequence that hybridizes under stringent conditions to a nucleotide sequence encoding a Sdc2 polypeptide, a fragment of a Sdc2 polypeptide, or an anti-Sdc2 antibody (or VH or VL region thereof) described herein at least 5 amino acid residues, at least 10 amino acid residues, at least 15 amino acid residues, at least 20 amino acid residues, at least 25 amino acid residues, at least 30 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues (see, e.g., Sambrook et al., Molecular Cloning: A Laboratory Manual (2001); and Maniatis et al., Molecular Cloning: A Laboratory Manual (1982)); or (c) a polypeptide encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to the nucleotide sequence encoding a Sdc2 polypeptide, a fragment of a Sdc2 polypeptide, or an anti-Sdc2 antibody (or VH or VL region thereof) described herein. A polypeptide with similar structure to a Sdc2 polypeptide, a fragment of a Sdc2 polypeptide, or an anti-Sdc2 antibody provided herein refers to a polypeptide that has a similar secondary, tertiary, or quaternary structure of a Sdc2 polypeptide, a fragment of a Sdc2 polypeptide, or an anti-Sdc2 antibody provided herein. The structure of a polypeptide can be determined by methods known to those skilled in the art, including but not limited to, X-ray crystallography, nuclear magnetic resonance, and crystallographic electron microscopy.

In the context of a polypeptide, the term “derivative” as used herein refers to a polypeptide that comprises an amino acid sequence of a Sdc2 polypeptide, a fragment of a Sdc2 polypeptide, or an antibody that binds to a Sdc2 polypeptide which has been altered by the introduction of amino acid residue substitutions, deletions, or additions. The term “derivative” as used herein also refers to a Sdc2 polypeptide, a fragment of a Sdc2 polypeptide, or an antibody that binds to a Sdc2 polypeptide which has been chemically modified, e.g., by the covalent attachment of any type of molecule to the polypeptide. For example, but not by way of limitation, a Sdc2 polypeptide, a fragment of a Sdc2 polypeptide, or an anti-Sdc2 antibody may be chemically modified, e.g., by glycosylation, acetylation, pegylation, phosphorylation, amidation, derivatization by known protecting/blocking groups, proteolytic cleavage, chemical cleavage, formulation, metabolic synthesis of tunicamycin, linkage to a cellular ligand or other protein, etc. The derivatives are modified in a manner that is different from naturally occurring or starting peptide or polypeptides, either in the type or location of the molecules attached. Derivatives further include deletion of one or more chemical groups which are naturally present on the peptide or polypeptide. Further, a derivative of a Sdc2 polypeptide, a fragment of a Sdc2 polypeptide, or an anti-Sdc2 antibody may contain one or more non-classical amino acids. A polypeptide derivative possesses a similar or identical function as a Sdc2 polypeptide, a fragment of a Sdc2 polypeptide, or an anti-Sdc2 antibody provided herein.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

### 6.2 Generation of Human Therapeutic Antibodies

Therapeutic use of humanized and fully human antibodies is preferable to the use of antibodies that possess murine or rat sequence regions (e.g., variable and/or constant regions) in that immune recognition of such murine or rat derived proteins as foreign can lead to the rapid clearance of the antibodies or can lead to the generation of an immune response against the antibody by a recipient subject. While isolating individual, antigen-specific plasma B cells from human subject is possible and increasing feasible with recent advances in high-throughput cell sorting and sequencing technologies, this method remains expensive and time consuming. Several other strategies have been developed to rapidly produce fully human or largely human antibodies against defined antigens for clinical development, including humanization and synthetic phage display.

“Humanization” is the process by which antibodies generated in non-human animal models (for instance mice) and screened for appropriate function and affinity are converted to mostly human sequences. Typically, this process involves the cloning of the antigen-specificity conferring amino acid sequences (e.g., the complementarity determining regions (CDRs)) into a fully human heavy and light chain backbone. In this way, the resulting mostly human hybrid antibody retains the antigen binding function conferred by the original mouse sequences. Discovery studies involving humanized antibodies benefit from the ease of working with small animal models in handling and immunization with the desired antigen. In certain embodiments, a mouse antibody generation platform utilizing a number of outbred strains (e.g., the PentaMice™ platform) was used to generate a wide variety of antibody clones against human, pig, and mouse Sdc2 protein. Those clones which passed screening, were humanized for further development.

Another strategy for rapidly producing antigen-specific fully human antibodies is the use of filamentous phage display libraries. Here, large libraries of scFv or Fab antibodies are produced by high throughput cloning methods and engineered onto the coat proteins of filamentous phage particles such that each viral particle displays a single unique antibody clone. The library of phage particles is then screen for high-affinity binding to an antigen of interest via panning. In certain embodiments, provided is a fully human anti-Sdc2 antibody and scFv clones which were screened from phage display libraries comprising naïve scFv clones (e.g., a XOMA phage display platform).

### 6.3 Anti-Syndecan-2 Antibodies

The invention is based, in part, on the discovery of one or more anti-syndecan-2 antibodies that modulate the signaling of syndecan-2, including by inhibiting syndecan-2 signaling, activating syndecan-2 signaling (i.e., in the absence of an agonist), or by potentiating syndecan-2 signaling in the presence of an agonist. In some embodiments, the antibody or antigen-binding fragment thereof may bind to the extracellular region of syndecan-2, and more specifically, to the region of the extracellular domain of syndecan-2 to which Dep-1 binds. The anti-syndecan-2 antibody may thereby inhibit or activate syndecan-2 signaling. In some embodiments, the antibody or antigen-binding fragment thereof may result in blocking or enhancing Dep-1 internalization, without blinding directly to the Dep-1 binding site of Sdc2. In some embodiments, the antibody or antigen-binding fragment thereof may bind to the Sdc2 extracellular domain and promote stabilization of the endothelial cell junctions, thereby resulting in inhibition of vascular permeability. The invention is also based in part on the discovery that modulation of syndecan-2 can be used to treat diseases whose pathogenesis is determined by vessel leakage or edema formation, including acute respiratory distress syndrome (ARDS) among others.

In one aspect, provided herein is an antibody that binds Sdc2. In some embodiments, the syndecan-2 protein to which the antibody or antigen-binding fragment thereof binds is of human, mouse, or porcine origin. In some embodiments, the antibody or antigen-binding fragment binds specifically to an epitope which is shared by human, mouse, and porcine syndecan-2 proteins or any combination thereof (i.e., human and mouse, mouse and porcine, or porcine and human).

Provided herein, in certain aspects is an anti-syndecan-2 antibody or antigen-binding fragment thereof, compositions comprising an anti-syndecan-2 antibody or antigen-binding fragment thereof, and methods for using the same. In some embodiments, the antigen-binding fragment is selected from the group consisting of a Fab, a single-chain variable fragment (scFv), or a single-domain antibody. In further embodiments, the antibody is a full-length antibody. In yet further embodiments, the antibody or antigen-binding fragment is a humanized antibody or an antigen-binding fragment thereof. In some embodiments, the antibody or antigen-binding fragment thereof is a fully human antibody or antigen-binding fragment thereof.

Regarding the anti-syndecan-2 antibody or antigen-binding fragment thereof and compositions comprising said antibody or antigen-binding fragment thereof, in various embodiments, an anti-syndecan-2 antibody or antigen-binding fragment thereof is provided; the anti-syndecan-2 antibody or antigen-binding fragment thereof comprising a heavy chain variable region and a light chain variable region, wherein the heavy chain variable region comprises an amino acid sequence having at least 80% sequence identity to at least one amino acid sequence selected from the group consisting of SEQ ID NOs: 1, 3, 5, 7, 9, 11, 13, 15, 17, 19, 21, and 23 and a light chain variable region comprising an amino acid sequence having at least 80% sequence identity to at least one amino acid selected from the group consisting of SEQ ID NOs: 2, 4, 6, 8, 10, 12, 14, 16, 18, 20, 22, and 24.

In one embodiment, the amino acid sequence comprised within the heavy chain variable region has at least 80%, 81%, 82%, 83%, 84%, 85%, 86%, 87%, 88%, 89%, 90%, 91%, 92% 93%, 94%, 95%, 96%, 97%, 98%, 99%, 99.5%, 99.6%, 99.7%, 99.8%, or 99.9% sequence identity, or has 100% sequence identity, to at least one selected from the group consisting of SEQ ID NOs: 1, 3, 5, 7, 9, 11, 13, 15, 17, 19, 21, and 23. In one embodiment, the heavy chain variable region consists of an amino acid sequence having at least 95% sequence identity to at least one selected from the group consisting of SEQ ID NOs: 1, 3, 5, 7, 9, 11, 13, 15, 17, 19, 21, and 23. In various embodiments herein, the amino acid sequence of the heavy chain variable region has at least 80%, 81%, 82%, 83%, 84%, 85%, 86%, 87%, 88%, 89%, 90%, 91%, 92% 93%, 94%, 95%, 96%, 97%, 98%, 99%, 99.5%, 99.6%, 99.7%, 99.8%, or 99.9% sequence identity, or has 100% sequence identity, to at least one selected from the group consisting of SEQ ID NOs: 1, 3, 5, 7, 9, 11, 13, 15, 17, 19, 21, and 23.

In one embodiment, the amino acid sequence comprised within the light chain variable region has at least 80%, 81% 82%, 83%, 84%, 85%, 86%, 87%, 88%, 89%, 90%, 91%, 92% 93%, 94%, 95%, 96%, 97%, 98%, 99%, 99.5%, 99.6%, 99.7%, 99.8%, or 99.9% sequence identity, or has 100% sequence identity, to at least one selected from the group consisting of SEQ ID NOs: 2, 4, 6, 8, 10, 12, 14, 16, 18, 20, 22, and 24. In one embodiment, the light chain variable region consists of an amino acid sequence having at least 95% sequence identity to at least one selected from the group consisting of SEQ ID NOs: 2, 4, 6, 8, 10, 12, 14, 16, 18, 20, 22, and 24. In various embodiments therein, the amino acid sequence of the light chain variable region has at least 80%, 81%, 82%, 83%, 84%, 85%, 86%, 87%, 88%, 89%, 90%, 91%, 92% 93%, 94%, 95%, 96%, 97%, 98%, 99%, 99.5%, 99.6%, 99.7%, 99.8%, or 99.9% sequence identity, or has 100% sequence identity, to at least one selected from the group consisting of SEQ ID NOs: 2, 4, 6, 8, 10, 12, 14, 16, 18, 20, 22, and 24.

In another embodiment, the amino acid sequence of the heavy chain variable region is selected from the group consisting of SEQ ID NOs: 1, 3, 5, 7, 9, 11, 13, 15, 17, 19, 21, and 23 and the amino acid sequence of the light chain variable region is selected from the group consisting of SEQ ID NOs: 2, 4, 6, 8, 10, 12, 14, 16, 18, 20, 22, and 24. In an additional embodiment, the heavy chain variable region consists of an amino acid sequence selected from the group consisting of SEQ ID NOs: 1, 3, 5, 7, 9, 11, 13, 15, 17, 19, 21, and 23 and the light chain variable region consists of an amino acid sequence selected from the group consisting of SEQ ID NOs: 2, 4, 6, 8, 10, 12, 14, 16, 18, 20, 22, and 24. In another embodiment, the amino acid sequence comprised within the heavy chain variable region and the amino acid sequence comprised within the light chain variable region are respectively selected from the group consisting of SEQ ID NOs: 1 and 2, 3 and 4, 5 and 6, 7 and 8, and 9 and 10. In an additional embodiment, the heavy chain variable region and the light chain variable region respectively consist of SEQ ID NOs: 1 and 2, 3 and 4, 5 and 6, 7 and 8, or 9 and 10.

In one embodiment, the anti-syndecan-2 antibody or antigen-binding fragment thereof is an IgA, an IgD, an IgE, an IgG, or an IgM. In one embodiment, the anti-syndecan-2 antibody or antigen-binding fragment thereof is an IgG1 or IgG4. In one embodiment, the anti-syndecan-2 antibody or antigen-binding fragment thereof is an IgG4. In one embodiment, the anti-syndecan-2 antibody or antigen binding fragment thereof is an IgE subclass, which includes a point mutation. In one embodiment, the point mutation in IgG4 is serine 228 to proline (S228P), which enhances the disulfide linkage in the core-hinge region.

In one embodiment, the anti-syndecan-2 antibody or antigen-binding fragment thereof is a selected from the group consisting of a full-length antibody, a Fab, and a single-chain variable fragment (scFv). In some embodiments, the anti-syndecan-2 antibody or antigen-binding fragment thereof is a humanized antibody or antigen-binding fragment thereof. In some embodiments, the anti-syndecan-2 antibody or antigen-binding fragment thereof is fully-human.

In another embodiment, the anti-syndecan-2 antibody or antigen-binding fragment thereof further comprises a heavy chain constant region and a light chain constant region, wherein the heavy chain constant region comprises an amino acid sequence having at least 80% sequence identity to SEQ ID NO: 1 and the light chain constant region comprises an amino acid sequence having at least 80% sequence identity to SEQ ID NO: 2. In another embodiment, the anti-syndecan-2 antibody or antigen-binding fragment thereof further comprises a heavy chain constant region and a light chain constant region, wherein the heavy chain constant region comprises an amino acid sequence having at least 80% sequence identity to SEQ ID NO: 7 and the light chain constant region comprises an amino acid sequence having at least 80% sequence identity to SEQ ID NO: 8. In another embodiment, the anti-syndecan-2 antibody or antigen-binding-fragment thereof further comprises a heavy chain constant region and a light chain constant region, wherein the heavy chain constant region comprises an amino acid sequence having at least 80% sequence identity to SEQ ID NO: 11 and the light chain constant region comprises an amino acid sequence having at least 80% sequence identity to SEQ ID NO: 12. In another embodiment, the anti-syndecan-2 antibody or antigen-binding-fragment thereof further comprises a heavy chain constant region and a light chain constant region, wherein the heavy chain constant region comprises an amino acid sequence having at least 80% sequence identity to SEQ ID NO: 13 and the light chain constant region comprises an amino acid sequence having at least 80% sequence identity to SEQ ID NO: 14. In another embodiment, the anti-syndecan-2 antibody or antigen-binding-fragment thereof further comprises a heavy chain constant region and a light chain constant region, wherein the heavy chain constant region comprises an amino acid sequence having at least 80% sequence identity to SEQ ID NO: 15 and the light chain constant region comprises an amino acid sequence having at least 80% sequence identity to SEQ ID NO: 16. In another embodiment, the anti-syndecan-2 antibody or antigen-binding-fragment thereof further comprises a heavy chain constant region and a light chain constant region, wherein the heavy chain constant region comprises an amino acid sequence having at least 80% sequence identity to SEQ ID NO: 17 and the light chain constant region comprises an amino acid sequence having at least 80% sequence identity to SEQ ID NO: 18.

In another aspect, provide is a single-chain variable fragment (scFv) comprising an antigen-binding domain that specifically binds to an epitope of syndecan-2 (Sdc2) protein, wherein the antigen-binding domain comprises a heavy chain variable region comprising an acid sequence having at least 80% sequence identity to at least one amino acid sequence selected from the group consisting of SEQ ID NOs: 1, 3, 5, 7, 9, 11, 13, 15, 17, 19, 21, and 23; and a light chain variable region comprising an amino acid sequence having at least 80% sequence identity to at least one amino acid selected from the group consisting of SEQ ID NOs: 2, 4, 6, 8, 10, 12, 14, 16, 18, 20, 22, and 24.

In various embodiments, the anti-syndecan-2 antibody or antigen-binding-fragment thereof further comprises a post-translational modification. Non-limiting examples of post-translational modifications include myristoylation, palmitoylation, stearoylation, glycosylation, the addition of heparan sulfate chains, and combinations thereof. One non-limiting example of glycosylation is the addition of heparan sulfate chains to the anti-syndecan-2 antibody.

Provided herein are anti-Sdc2 antibodies or antigen-binding fragments thereof, nucleic acids and expression vectors encoding the antibodies, recombinant cells containing the vectors, and compositions comprising the antibodies. Methods of making the antibodies, and methods of using the antibodies to treat diseases are also provided. The antibodies disclosed herein possess one or more desirable functional properties, including but not limited to high-affinity binding to Sdc2 or high specificity to Sdc2. In certain embodiments, the antibodies disclosed herein possess the ability to treat or prevent a disease or disorder when administered to a subject alone or in combination with other therapies.

In one aspect, provided herein is an antibody that binds to Sdc2. In some embodiments, the antibody comprises a heavy chain variable region and a light chain variable region. In some embodiments, the Sdc2 antibody is a single domain antibody or nanobody. In some embodiments, the Sdc2 antibody is not a single domain antibody or nanobody. In some embodiments, the Sdc2 antibody is a humanized antibody. In certain embodiments, the Sdc2 antibody is a fully human antibody.

In one embodiment, the present disclosure provides anti-Sdc2 antibodies that may find use herein as therapeutic agents. In another embodiment, the present disclosure provides anti-Sdc2 antibodies that may find use herein as diagnostic agents. Exemplary antibodies include polyclonal, monoclonal, humanized, human, bispecific, and heteroconjugate antibodies, as well as variants thereof having improved affinity or other properties.

In certain embodiments, provided herein is a Sdc2 antibody comprising a VH region, VL region, VH CDR1, VH CDR2, VH CDR3, VL CDR1, VL CDR2, and/or VL CDR3 of any one of the antibodies described herein. In some embodiments, provided herein is a Sdc2 antibody comprising a VH region of any one of the antibodies described herein. In some embodiments, provided herein is a Sdc2 antibody comprising a VL region of any one of the antibodies described herein. In some embodiments, provided herein is a Sdc2 antibody comprising a VH region of any one of the antibodies described herein, and a VL region of any one of the antibodies described herein. In some embodiments, provided herein is a Sdc2 antibody comprising a VH CDR1, VH CDR2, and VH CDR3 of any one of the antibodies described herein. In some embodiments, provided herein is a Sdc2 antibody comprising a VL CDR1, VL CDR2, and VL CDR3 of any one of the antibodies described herein. In some embodiments, provided herein is a Sdc2 antibody comprising a VH CDR1, VH CDR2, and VH CDR3 of any one of the antibodies described herein; and a VL CDR1, VL CDR2, and VL CDR3 of any one of the antibodies described herein. Representative VH and VL amino acid sequences, including VH CDR1, VH CDR2, VH CDR3, VL CDR1, VL CDR2 and VL CDR3 amino acid sequences, of anti-Sdc2 antibodies provided herein are provided in Tables 1 and 3-8 below.

Accordingly, in some embodiments, the isolated antibody or functional fragment thereof provided herein comprises one, two, and/or three heavy chain CDRs and/or one, two, and/or three light chain CDRs from: (a) the antibody 20-H19-AB, (b) the antibody TP-43327F, (c) the antibody TP-43329F, (d) the antibody 8-G17-A, (e) the antibody 6-N03-A, (f) the antibody R3-P3-C11, (g) the antibody R4M-P3-E06, (h) the antibody R3-P3-E09, (i) the antibody R3-P1-C02, (j) the antibody R3-P3-A12, (k) the antibody R4M-P3-A12, or (l) the antibody R4M-P1-A10, as shown in Tables 1 and 3-8 below.

In some embodiments, the antibody specifically binds Sdc2. In some embodiments, the Sdc2 is present on the surface of an endothelial cell. In some embodiments, the Sdc2 is present on the surface of a neural cell.

In some embodiments, the antibody is a humanized antibody. In certain embodiments, the antibody is an IgG antibody. In other embodiments, the IgG antibody is an IgG1, IgG2, IgG3, or IgG4 antibody. In one embodiment, the IgG antibody is an IgG1 antibody. In one embodiment, the IgG antibody is an IgG2 antibody. In one embodiment, the IgG antibody is an IgG3 antibody. In one embodiment, the IgG antibody is an IgG4 antibody. In some embodiments, the antibody is a bispecific antibody. In certain embodiments, the antibody is multivalent. In other embodiments, the antibody is capable of binding at least three antigens. In some embodiments, the antibody is capable of binding at least five antigens.

In certain embodiments, provided is a Sdc2 antibody that is an intact antibody. In other embodiments, provided is a Sdc2 antibody is an antigen binding fragment of the Sdc2 antibody. In some embodiments, the antigen binding fragment of the Sdc2 antibody is a functional fragment.

In some embodiments, the antigen binding fragment is a diabody. In some embodiments, the antigen binding fragment is a Fab. In some embodiments, the antigen binding fragment is a Fab′. In some embodiments, the antigen binding fragment is a F(ab′)2. In some embodiments, the antigen binding fragment is a Fv fragment. In some embodiments, the antigen binding fragment is a disulfide stabilized Fv fragment (dsFv). In some embodiments, the antigen binding fragment is a (dsFv)₂. In some embodiments, the antigen binding fragment is a bispecific dsFv (dsFv-dsFv′). In some embodiments, the antigen binding fragment is a disulfide stabilized diabody (ds diabody). In some embodiments, the antigen binding fragment is a single-chain antibody molecule (scFv). In some embodiments, the antigen binding fragment is a single domain antibody (sdAb). In some embodiments, the antigen binding fragment is an scFv dimer (bivalent diabody). In some embodiments, the antigen binding fragment is a multispecific antibody formed from a portion of an antibody comprising one or more CDRs. In some embodiments, the antigen binding fragment is a camelized single domain antibody. In some embodiments, the antigen binding fragment is a nanobody. In some embodiments, the antigen binding fragment is a domain antibody. In some embodiments, the antigen binding fragment is a bivalent domain antibody. In some embodiments, the antigen binding fragment is an antibody fragment that binds to an antigen but does not comprise a complete antibody structure.

In specific embodiments, the Sdc2 antibody comprises a VH region and a VL region. In some embodiments, the Sdc2 antibody is a single chain antibody. In some embodiments, the Sdc2 antibody is a single domain antibody. In some embodiments, the Sdc2 antibody is a nanobody. In certain embodiments, the Sdc2 antibody is a VHH antibody. In certain embodiments, the Sdc2 antibody is a llama antibody. In some embodiments, the Sdc2 antibody is not a single chain antibody. In some embodiments, the Sdc2 antibody is not a single domain antibody. In some embodiments, the Sdc2 antibody is not a nanobody. In certain embodiments, the Sdc2 antibody is not a VHH antibody. In certain embodiments, the Sdc2 antibody is not a llama antibody. In some embodiments, the Sdc2 antibody is a multispecific antibody. In other embodiments, the Sdc2 is a bispecific antibody. In certain embodiments, the multispecific antibody comprises an antigen binding fragment of a Sdc2 antibody provided herein. In other embodiments, the bispecific antibody comprises an antigen binding fragment of a Sdc2 antibody provided herein. In some embodiments, the Sdc2 antibody is an agonistic antibody. In certain embodiments, the Sdc2 antibody activates T cells. In other embodiments, the Sdc2 antibody is an antagonistic antibody. In certain embodiments, the Sdc2 antibody inactivates T cells. In some embodiments, the Sdc2 antibody blocks activation of T cells. In some embodiments, the Sdc2 antibody modulates the activity of T cells. In some embodiments, the Sdc2 antibody neither activates nor inactivates the activity of T cells. In specific embodiments, the T cells are human T cells.

In some embodiments, the VH CDR1, VH CDR2, VH CDR3, VL CDR1, VL CDR2, and VL CDR3 sequences are according to the Kabat numbering system. In some embodiments, the VH CDR1, VH CDR2, VH CDR3, VL CDR1, VL CDR2, and VL CDR3 sequences are according to the Chothia numbering system. In some embodiments, the VH CDR1, VH CDR2, VH CDR3, VL CDR1, VL CDR2, and VL CDR3 sequences are according to the Exemplary numbering system. In some embodiments, the VH CDR1, VH CDR2, VH CDR3, VL CDR1, VL CDR2, and VL CDR3 sequences are according to the Contact numbering system. In some embodiments, the VH CDR1, VH CDR2, VH CDR3, VL CDR1, VL CDR2, and VL CDR3 sequences are according to the IMGT numbering system. In some embodiments, the VH CDR1, VH CDR2, VH CDR3, VL CDR1, VL CDR2, and VL CDR3 sequences are according to the AbM numbering system. Exemplary sets of 6 CDRs (VH CDR1-3 and VL CDR1-3) of certain antibody embodiments are provided herein. Other sets of CDRs are contemplated and within the scope of the antibody embodiments provided herein.

In one aspect, provided herein is an antibody that binds Sdc2, wherein the antibody that binds to Sdc2 is antibody clone 20-H19-AB. In one aspect, provided herein is an antibody that binds Sdc2, wherein the antibody comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:61; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:62. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 31, 32 and 33, respectively; and (ii) a VL comprising a VL CDR1, a VL CDR2, and a VL CDR3 having an amino acid sequence of SEQ ID NOs: 34, 35 and 36, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 37, 38 and 39, respectively; and (ii) a VL comprising a VL CDR1, a VL CDR2, and a VL CDR3 having an amino acid sequence of SEQ ID NOs: 40, 41 and 42, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 43, 44 and 45, respectively; and (ii) a VL comprising a VL CDR1, a VL CDR2, and a VL CDR3 having an amino acid sequence of SEQ ID NOs: 46, 47 and 48, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 49, 50 and 51, respectively; and (ii) a VL comprising a VL CDR1, a VL CDR2, and a VL CDR3 having an amino acid sequence of SEQ ID NOs: 52, 53 and 54, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 55, 56 and 57, respectively; and (ii) a VL comprising a VL CDR1, a VL CDR2, and a VL CDR3 having an amino acid sequence of SEQ ID NOs: 58, 59 and 60, respectively. In one aspect, provided herein is an antibody that binds Sdc2, wherein the antibody comprises a VH having an amino acid sequence having at least 95% identity to an amino acid sequence of SEQ ID NO:61. In one aspect, provided herein is an antibody that binds Sdc2, wherein the antibody comprises a VL having an amino acid sequence having at least 95% identity to an amino acid sequence of SEQ ID NO:62. In one aspect, provided herein is an antibody that binds Sdc2, wherein the antibody comprises (i) a VH having an amino acid sequence having at least 95% identity to an amino acid sequence of SEQ ID NO:61; and (ii) a VL having an amino acid sequence having at least 95% identity to an amino acid sequence of SEQ ID NO:62. In one aspect, provided herein is an antibody that binds Sdc2, wherein the antibody comprises a VH having an amino acid sequence SEQ ID NO:61. In one aspect, provided herein is an antibody that binds Sdc2, wherein the antibody comprises a VL having an amino acid sequence SEQ ID NO:62. In one aspect, provided herein is an antibody that binds Sdc2, wherein the antibody comprises (i) a VH having an amino acid sequence SEQ ID NO:61; and (ii) a VL having an amino acid sequence SEQ ID NO:62. In one aspect, provided herein is an antibody that binds Sdc2, wherein the antibody comprises a heavy chain having an amino acid sequence having at least 95% identity to an amino acid sequence of SEQ ID NO:63. In one aspect, provided herein is an antibody that binds Sdc2, wherein the antibody comprises a light chain having an amino acid sequence having at least 95% identity to an amino acid sequence of SEQ ID NO:64. In one aspect, provided herein is an antibody that binds Sdc2, wherein the antibody comprises (i) a heavy chain having an amino acid sequence having at least 95% identity to an amino acid sequence of SEQ ID NO:63; and (ii) a light chain having an amino acid sequence having at least 95% identity to an amino acid sequence of SEQ ID NO:64. In one aspect, provided herein is an antibody that binds Sdc2, wherein the antibody comprises a heavy chain having an amino acid sequence SEQ ID NO:63. In one aspect, provided herein is an antibody that binds Sdc2, wherein the antibody comprises a light chain having an amino acid sequence SEQ ID NO:64. In one aspect, provided herein is an antibody that binds Sdc2, wherein the antibody comprises (i) a heavy chain having an amino acid sequence SEQ ID NO:63; and (ii) a light chain having an amino acid sequence SEQ ID NO: 64.

In one aspect, provided herein is an antibody that binds Sdc2, wherein the antibody that binds to Sdc2 is antibody clone TP-43327F. In one aspect, provided herein is an antibody that binds Sdc2, wherein the antibody comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:95; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:96. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 65, 66 and 67, respectively; and (ii) a VL comprising a VL CDR1, a VL CDR2, and a VL CDR3 having an amino acid sequence of SEQ ID NOs: 68, 89 and 70, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 71, 72 and 73, respectively; and (ii) a VL comprising a VL CDR1, a VL CDR2, and a VL CDR3 having an amino acid sequence of SEQ ID NOs: 74, 75 and 76, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 77, 78 and 79, respectively; and (ii) a VL comprising a VL CDR1, a VL CDR2, and a VL CDR3 having an amino acid sequence of SEQ ID NOs: 80, 81 and 82, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 83, 84 and 85, respectively; and (ii) a VL comprising a VL CDR1, a VL CDR2, and a VL CDR3 having an amino acid sequence of SEQ ID NOs: 86, 87 and 88, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 89, 90 and 91, respectively; and (ii) a VL comprising a VL CDR1, a VL CDR2, and a VL CDR3 having an amino acid sequence of SEQ ID NOs: 92, 93 and 94, respectively. In one aspect, provided herein is an antibody that binds Sdc2, wherein the antibody comprises a VH having an amino acid sequence having at least 95% identity to an amino acid sequence of SEQ ID NO:95. In one aspect, provided herein is an antibody that binds Sdc2, wherein the antibody comprises a VL having an amino acid sequence having at least 95% identity to an amino acid sequence of SEQ ID NO:96. In one aspect, provided herein is an antibody that binds Sdc2, wherein the antibody comprises (i) a VH having an amino acid sequence having at least 95% identity to an amino acid sequence of SEQ ID NO:95; and (ii) a VL having an amino acid sequence having at least 95% identity to an amino acid sequence of SEQ ID NO:96. In one aspect, provided herein is an antibody that binds Sdc2, wherein the antibody comprises a VH having an amino acid sequence SEQ ID NO:95. In one aspect, provided herein is an antibody that binds Sdc2, wherein the antibody comprises a VL having an amino acid sequence SEQ ID NO:96. In one aspect, provided herein is an antibody that binds Sdc2, wherein the antibody comprises (i) a VH having an amino acid sequence SEQ ID NO:95; and (ii) a VL having an amino acid sequence SEQ ID NO:96. In one aspect, provided herein is an antibody that binds Sdc2, wherein the antibody comprises a heavy chain having an amino acid sequence having at least 95% identity to an amino acid sequence of SEQ ID NO:97. In one aspect, provided herein is an antibody that binds Sdc2, wherein the antibody comprises a light chain having an amino acid sequence having at least 95% identity to an amino acid sequence of SEQ ID NO:98. In one aspect, provided herein is an antibody that binds Sdc2, wherein the antibody comprises (i) a heavy chain having an amino acid sequence having at least 95% identity to an amino acid sequence of SEQ ID NO:97; and (ii) a light chain having an amino acid sequence having at least 95% identity to an amino acid sequence of SEQ ID NO:98. In one aspect, provided herein is an antibody that binds Sdc2, wherein the antibody comprises a heavy chain having an amino acid sequence SEQ ID NO:97. In one aspect, provided herein is an antibody that binds Sdc2, wherein the antibody comprises a light chain having an amino acid sequence SEQ ID NO:98. In one aspect, provided herein is an antibody that binds Sdc2, wherein the antibody comprises (i) a heavy chain having an amino acid sequence SEQ ID NO:97; and (ii) a light chain having an amino acid sequence SEQ ID NO: 98.

In one aspect, provided herein is an antibody that binds Sdc2, wherein the antibody that binds to Sdc2 is antibody clone TP-43329F. In one aspect, provided herein is an antibody that binds Sdc2, wherein the antibody comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 129; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 130. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 99, 100 and 101, respectively; and (ii) a VL comprising a VL CDR1, a VL CDR2, and a VL CDR3 having an amino acid sequence of SEQ ID NOs: 102,103 and 104, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 105, 106 and 107, respectively; and (ii) a VL comprising a VL CDR1, a VL CDR2, and a VL CDR3 having an amino acid sequence of SEQ ID NOs: 108, 109 and 110, comprises (i) a VH comprising a VH CDR1, a VH CDR2, and a VH CDR3 having an amino acid sequence of SEQ ID NOs: 111, 112 and 113, respectively; and (ii) a VL comprising a VL CDR1, a VL CDR2, and a VL CDR3 having an amino acid sequence of SEQ ID NOs: 114, 115 and 116, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 117, 118 and 119, respectively; and (ii) a VL comprising a VL CDR1, a VL CDR2, and a VL CDR3 having an amino acid sequence of SEQ ID NOs: 120, 121 and 122, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 123, 124 and 125, respectively; and (ii) a VL comprising a VL CDR1, a VL CDR2, and a VL CDR3 having an amino acid sequence of SEQ ID NOs: 126, 127 and 128, respectively. In one aspect, provided herein is an antibody that binds Sdc2, wherein the antibody comprises a VH having an amino acid sequence having at least 95% identity to an amino acid sequence of SEQ ID NO:129. In one aspect, provided herein is an antibody that binds Sdc2, wherein the antibody comprises a VL having an amino acid sequence having at least 95% identity to an amino acid sequence of SEQ ID NO: 130. In one aspect, provided herein is an antibody that binds Sdc2, wherein the antibody comprises (i) a VH having an amino acid sequence having at least 95% identity to an amino acid sequence of SEQ ID NO: 129; and (ii) a VL having an amino acid sequence having at least 95% identity to an amino acid sequence of SEQ ID NO: 130. In one aspect, provided herein is an antibody that binds Sdc2, wherein the antibody comprises a VH having an amino acid sequence SEQ ID NO:129. In one aspect, provided herein is an antibody that binds Sdc2, wherein the antibody comprises a VL having an amino acid sequence SEQ ID NO:130. In one aspect, provided herein is an antibody that binds Sdc2, wherein the antibody comprises a VH having an amino acid sequence SEQ ID NO: 129; and (ii) a VL having an amino acid sequence SEQ ID NO:130. In one aspect, provided herein is an antibody that binds Sdc2, wherein the antibody comprises a heavy chain having an amino acid sequence having at least 95% identity to an amino acid sequence of SEQ ID NO: 131. In one aspect, provided herein is an antibody that binds Sdc2, wherein the antibody comprises a light chain having an amino acid sequence having at least 95% identity to an amino acid sequence of SEQ ID NO: 132. In one aspect, provided herein is an antibody that binds Sdc2, wherein the antibody comprises a heavy chain having an amino acid sequence having at least 95% identity to an amino acid sequence of SEQ ID NO: 131; and (ii) a light chain having an amino acid sequence having at least 95% identity to an amino acid sequence of SEQ ID NO:132. In one aspect, provided herein is an antibody that binds Sdc2, wherein the antibody comprises a heavy chain having an amino acid sequence SEQ ID NO: 131. In one aspect, amino acid sequence SEQ ID NO: 132. In one aspect, provided herein is an antibody that binds Sdc2, wherein the antibody comprises a heavy chain having an amino acid sequence SEQ ID NO:131; and (ii) a light chain having an amino acid sequence SEQ ID NO:132.

In one aspect, provided herein is an antibody that binds Sdc2, wherein the antibody that binds to Sdc2 is antibody clone 8-G17A. In one aspect, provided herein is an antibody that binds Sdc2, wherein the antibody comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 163; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 164. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 133, 134 and 135, respectively; and (ii) a VL comprising a VL CDR1, a VL CDR2, and a VL CDR3 having an amino acid sequence of SEQ ID NOs: 136, 137 and 138, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 139, 140 and 141, respectively; and (ii) a VL comprising a VL CDR1, a VL CDR2, and a VL CDR3 having an amino acid sequence of SEQ ID NOs: 142, 143 and 144, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 145, 146 and 147, respectively; and (ii) a VL comprising a VL CDR1, a VL CDR2, and a VL CDR3 having an amino acid sequence of SEQ ID NOs: 148, 149 and 150, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 151, 152 and 153, respectively; and (ii) a VL comprising a VL CDR1, a VL CDR2, and a VL CDR3 having an amino acid sequence of SEQ ID NOs: 154, 155 and 156, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 157, 158 and 159, respectively; and (ii) a VL comprising a VL CDR1, a VL CDR2, and a VL CDR3 having an amino acid sequence of SEQ ID NOs: 160, 161 and 162, respectively. In one aspect, provided herein is an antibody that binds Sdc2, wherein the antibody comprises a VH having an amino acid sequence having at least 95% identity to an amino acid sequence of SEQ ID NO:163. In one aspect, provided herein is an antibody that binds Sdc2, wherein the antibody comprises a VL having an amino acid sequence having at least 95% identity to an amino acid sequence of SEQ ID NO: 164. In one aspect, provided herein is an antibody that identity to an amino acid sequence of SEQ ID NO: 163; and (ii) a VL having an amino acid sequence having at least 95% identity to an amino acid sequence of SEQ ID NO: 164. In one aspect, provided herein is an antibody that binds Sdc2, wherein the antibody comprises a VH having an amino acid sequence SEQ ID NO:163. In one aspect, provided herein is an antibody that binds Sdc2, wherein the antibody comprises a VL having an amino acid sequence SEQ ID NO:164. In one aspect, provided herein is an antibody that binds Sdc2, wherein the antibody comprises (i) a VH having an amino acid sequence SEQ ID NO: 163; and (ii) a VL having an amino acid sequence SEQ ID NO:164. In one aspect, provided herein is an antibody that binds Sdc2, wherein the antibody comprises a heavy chain having an amino acid sequence having at least 95% identity to an amino acid sequence of SEQ ID NO: 165. In one aspect, provided herein is an antibody that binds Sdc2, wherein the antibody comprises a light chain having an amino acid sequence having at least 95% identity to an amino acid sequence of SEQ ID NO: 166. In one aspect, provided herein is an antibody that binds Sdc2, wherein the antibody comprises (i) a heavy chain having an amino acid sequence having at least 95% identity to an amino acid sequence of SEQ ID NO: 165; and (ii) a light chain having an amino acid sequence having at least 95% identity to an amino acid sequence of SEQ ID NO:166. In one aspect, provided herein is an antibody that binds Sdc2, wherein the antibody comprises a heavy chain having an amino acid sequence SEQ ID NO: 165. In one aspect, provided herein is an antibody that binds Sdc2, wherein the antibody comprises a light chain having an amino acid sequence SEQ ID NO:166. In one aspect, provided herein is an antibody that binds Sdc2, wherein the antibody comprises (i) a heavy chain having an amino acid sequence SEQ ID NO: 165; and (ii) a light chain having an amino acid sequence SEQ ID NO:166.

In one aspect, provided herein is an antibody that binds Sdc2, wherein the antibody that binds to Sdc2 is antibody clone 6-N03-A. In one aspect, provided herein is an antibody that binds Sdc2, wherein the antibody comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 197; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 198. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 167, 168 and 169, respectively; and (ii) a VL comprising a VL CDR1, a VL CDR2, and a VL CDR3 having an amino acid sequence of SEQ ID NOs: 170, 171 and 172, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 173, 174 and 175, respectively; and (ii) a VL comprising a VL CDR1, a VL CDR2, and a VL CDR3 having an amino acid sequence of SEQ ID NOs: 176, 177 and 178, comprises (i) a VH comprising a VH CDR1, a VH CDR2, and a VH CDR3 having an amino acid sequence of SEQ ID NOs: 179, 180 and 181, respectively; and (ii) a VL comprising a VL CDR1, a VL CDR2, and a VL CDR3 having an amino acid sequence of SEQ ID NOs: 182, 183 and 184, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 185, 186 and 187, respectively; and (ii) a VL comprising a VL CDR1, a VL CDR2, and a VL CDR3 having an amino acid sequence of SEQ ID NOs: 188, 189 and 190, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 191, 192 and 193, respectively; and (ii) a VL comprising a VL CDR1, a VL CDR2, and a VL CDR3 having an amino acid sequence of SEQ ID NOs: 194, 195 and 196, respectively. In one aspect, provided herein is an antibody that binds Sdc2, wherein the antibody comprises a VH having an amino acid sequence having at least 95% identity to an amino acid sequence of SEQ ID NO:197. In one aspect, provided herein is an antibody that binds Sdc2, wherein the antibody comprises a VL having an amino acid sequence having at least 95% identity to an amino acid sequence of SEQ ID NO: 198. In one aspect, provided herein is an antibody that binds Sdc2, wherein the antibody comprises (i) a VH having an amino acid sequence having at least 95% identity to an amino acid sequence of SEQ ID NO: 197; and (ii) a VL having an amino acid sequence having at least 95% identity to an amino acid sequence of SEQ ID NO: 198. In one aspect, provided herein is an antibody that binds Sdc2, wherein the antibody comprises a VH having an amino acid sequence SEQ ID NO:197. In one aspect, provided herein is an antibody that binds Sdc2, wherein the antibody comprises a VL having an amino acid sequence SEQ ID NO:198. In one aspect, provided herein is an antibody that binds Sdc2, wherein the antibody comprises (i) a VH having an amino acid sequence SEQ ID NO: 197; and (ii) a VL having an amino acid sequence SEQ ID NO:198. In one aspect, provided herein is an antibody that binds Sdc2, wherein the antibody comprises a heavy chain having an amino acid sequence having at least 95% identity to an amino acid sequence of SEQ ID NO: 199. In one aspect, provided herein is an antibody that binds Sdc2, wherein the antibody comprises a light chain having an amino acid sequence having at least 95% identity to an amino acid sequence of SEQ ID NO:200. In one aspect, provided herein is an antibody that binds Sdc2, wherein the antibody comprises (i) a heavy chain having an amino acid sequence having at least 95% identity to an amino acid sequence of SEQ ID NO: 199; and (ii) a light chain having an amino acid sequence having at least 95% identity to an amino acid sequence of SEQ ID NO:200. In one aspect, provided herein is an antibody that binds Sdc2, wherein the antibody comprises a heavy chain having an amino acid sequence SEQ ID NO: 199. In one aspect, amino acid sequence SEQ ID NO:200. In one aspect, provided herein is an antibody that binds Sdc2, wherein the antibody comprises (i) a heavy chain having an amino acid sequence SEQ ID NO: 199; and (ii) a light chain having an amino acid sequence SEQ ID NO:200.

In one aspect, provided herein is an antibody that binds Sdc2, wherein the antibody that binds to Sdc2 is antibody clone R3-P3-C11. In one aspect, provided herein is an antibody that binds Sdc2, wherein the antibody comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:231; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:232. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 201, 202 and 203, respectively; and (ii) a VL comprising a VL CDR1, a VL CDR2, and a VL CDR3 having an amino acid sequence of SEQ ID NOs: 204, 205 and 206, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 207, 208 and 209, respectively; and (ii) a VL comprising a VL CDR1, a VL CDR2, and a VL CDR3 having an amino acid sequence of SEQ ID NOs: 210, 211 and 212, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 213, 214 and 215, respectively; and (ii) a VL comprising a VL CDR1, a VL CDR2, and a VL CDR3 having an amino acid sequence of SEQ ID NOs: 216, 217 and 218, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 219, 220 and 221, respectively; and (ii) a VL comprising a VL CDR1, a VL CDR2, and a VL CDR3 having an amino acid sequence of SEQ ID NOs: 222, 223 and 224, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 225, 226 and 227, respectively; and (ii) a VL comprising a VL CDR1, a VL CDR2, and a VL CDR3 having an amino acid sequence of SEQ ID NOs: 228, 229 and 230, respectively. In one aspect, provided herein is an antibody that binds Sdc2, wherein the antibody comprises a VH having an amino acid sequence having at least 95% identity to an amino acid sequence of SEQ ID NO:231. In one aspect, provided herein is an antibody that binds Sdc2, wherein the antibody comprises a VL having an amino acid sequence having at least 95% identity to an amino acid sequence of SEQ ID NO:232. In one aspect, provided herein is an antibody that identity to an amino acid sequence of SEQ ID NO:231; and (ii) a VL having an amino acid sequence having at least 95% identity to an amino acid sequence of SEQ ID NO:232. In one aspect, provided herein is an antibody that binds Sdc2, wherein the antibody comprises a VH having an amino acid sequence SEQ ID NO:231. In one aspect, provided herein is an antibody that binds Sdc2, wherein the antibody comprises a VL having an amino acid sequence SEQ ID NO:232. In one aspect, provided herein is an antibody that binds Sdc2, wherein the antibody comprises (i) a VH having an amino acid sequence SEQ ID NO: 231; and (ii) a VL having an amino acid sequence SEQ ID NO:232. In one aspect, provided herein is an antibody that binds Sdc2, wherein the antibody comprises a heavy chain having an amino acid sequence having at least 95% identity to an amino acid sequence of SEQ ID NO:233. In one aspect, provided herein is an antibody that binds Sdc2, wherein the antibody comprises a light chain having an amino acid sequence having at least 95% identity to an amino acid sequence of SEQ ID NO:234. In one aspect, provided herein is an antibody that binds Sdc2, wherein the antibody comprises (i) a heavy chain having an amino acid sequence having at least 95% identity to an amino acid sequence of SEQ ID NO: 233; and (ii) a light chain having an amino acid sequence having at least 95% identity to an amino acid sequence of SEQ ID NO:234. In one aspect, provided herein is an antibody that binds Sdc2, wherein the antibody comprises a heavy chain having an amino acid sequence SEQ ID NO:233. In one aspect, provided herein is an antibody that binds Sdc2, wherein the antibody comprises a light chain having an amino acid sequence SEQ ID NO:234. In one aspect, provided herein is an antibody that binds Sdc2, wherein the antibody comprises (i) a heavy chain having an amino acid sequence SEQ ID NO:233; and (ii) a light chain having an amino acid sequence SEQ ID NO:234.

In one aspect, provided herein is an antibody that binds Sdc2, wherein the antibody that binds to Sdc2 is antibody clone R4M-P3-E06. In one aspect, provided herein is an antibody that binds Sdc2, wherein the antibody comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:265; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:266. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 235, 236 and 237, respectively; and (ii) a VL comprising a VL CDR1, a VL CDR2, and a VL CDR3 having an amino acid sequence of SEQ ID NOs: 238, 239 and 240, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 241, 242 and 243, respectively; and (ii) a VL comprising a VL CDR1, a VL CDR2, and a VL CDR3 having an amino acid sequence of SEQ ID NOs: 244, 245 and 246, comprises (i) a VH comprising a VH CDR1, a VH CDR2, and a VH CDR3 having an amino acid sequence of SEQ ID NOs: 247, 248 and 249, respectively; and (ii) a VL comprising a VL CDR1, a VL CDR2, and a VL CDR3 having an amino acid sequence of SEQ ID NOs: 250, 251 and 252, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 253, 254 and 255, respectively; and (ii) a VL comprising a VL CDR1, a VL CDR2, and a VL CDR3 having an amino acid sequence of SEQ ID NOs: 256, 257 and 258, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 259, 260 and 261, respectively; and (ii) a VL comprising a VL CDR1, a VL CDR2, and a VL CDR3 having an amino acid sequence of SEQ ID NOs: 262, 263 and 264, respectively. In one aspect, provided herein is an antibody that binds Sdc2, wherein the antibody comprises a VH having an amino acid sequence having at least 95% identity to an amino acid sequence of SEQ ID NO:265. In one aspect, provided herein is an antibody that binds Sdc2, wherein the antibody comprises a VL having an amino acid sequence having at least 95% identity to an amino acid sequence of SEQ ID NO 266. In one aspect, provided herein is an antibody that binds Sdc2, wherein the antibody comprises (i) a VH having an amino acid sequence having at least 95% identity to an amino acid sequence of SEQ ID NO:265; and (ii) a VL having an amino acid sequence having at least 95% identity to an amino acid sequence of SEQ ID NO 266. In one aspect, provided herein is an antibody that binds Sdc2, wherein the antibody comprises a VH having an amino acid sequence SEQ ID NO:265. In one aspect, provided herein is an antibody that binds Sdc2, wherein the antibody comprises a VL having an amino acid sequence SEQ ID NO:266. In one aspect, provided herein is an antibody that binds Sdc2, wherein the antibody comprises (i) a VH having an amino acid sequence SEQ ID NO: 265; and (ii) a VL having an amino acid sequence SEQ ID NO:266. In one aspect, provided herein is an antibody that binds Sdc2, wherein the antibody comprises a heavy chain having an amino acid sequence having at least 95% identity to an amino acid sequence of SEQ ID NO:267. In one aspect, provided herein is an antibody that binds Sdc2, wherein the antibody comprises a light chain having an amino acid sequence having at least 95% identity to an amino acid sequence of SEQ ID NO:268. In one aspect, provided herein is an antibody that binds Sdc2, wherein the antibody comprises (i) a heavy chain having an amino acid sequence having at least 95% identity to an amino acid sequence of SEQ ID NO: 267; and (ii) a light chain having an amino acid sequence having at least 95% identity to an amino acid sequence of SEQ ID NO:268. In one aspect, provided herein is an antibody that binds Sdc2, wherein the antibody comprises a heavy chain having an amino acid sequence SEQ ID NO:267. In one aspect, amino acid sequence SEQ ID NO:268. In one aspect, provided herein is an antibody that binds Sdc2, wherein the antibody comprises (i) a heavy chain having an amino acid sequence SEQ ID NO:267; and (ii) a light chain having an amino acid sequence SEQ ID NO:268.

In one aspect, provided herein is an antibody that binds Sdc2, wherein the antibody that binds to Sdc2 is antibody clone R3-P3-E09. In one aspect, provided herein is an antibody that binds Sdc2, wherein the antibody comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:299; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:300. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 269, 270 and 271, respectively; and (ii) a VL comprising a VL CDR1, a VL CDR2, and a VL CDR3 having an amino acid sequence of SEQ ID NOs: 272, 273 and 274, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 275, 276 and 277, respectively; and (ii) a VL comprising a VL CDR1, a VL CDR2, and a VL CDR3 having an amino acid sequence of SEQ ID NOs: 278, 279 and 280, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 281, 282 and 283, respectively; and (ii) a VL comprising a VL CDR1, a VL CDR2, and a VL CDR3 having an amino acid sequence of SEQ ID NOs: 284, 285 and 286, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 287, 288 and 289, respectively; and (ii) a VL comprising a VL CDR1, a VL CDR2, and a VL CDR3 having an amino acid sequence of SEQ ID NOs: 290, 292 and 292, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 293, 294 and 295, respectively; and (ii) a VL comprising a VL CDR1, a VL CDR2, and a VL CDR3 having an amino acid sequence of SEQ ID NOs: 296, 297 and 298, respectively. In one aspect, provided herein is an antibody that binds Sdc2, wherein the antibody comprises a VH having an amino acid sequence having at least 95% identity to an amino acid sequence of SEQ ID NO:299. In one aspect, provided herein is an antibody that binds Sdc2, wherein the antibody comprises a VL having an amino acid sequence having at least 95% identity to an amino acid sequence of SEQ ID NO:300. In one aspect, provided herein is an antibody that identity to an amino acid sequence of SEQ ID NO:299; and (ii) a VL having an amino acid sequence having at least 95% identity to an amino acid sequence of SEQ ID NO:300. In one aspect, provided herein is an antibody that binds Sdc2, wherein the antibody comprises a VH having an amino acid sequence SEQ ID NO:299. In one aspect, provided herein is an antibody that binds Sdc2, wherein the antibody comprises a VL having an amino acid sequence SEQ ID NO:300. In one aspect, provided herein is an antibody that binds Sdc2, wherein the antibody comprises (i) a VH having an amino acid sequence SEQ ID NO: 299; and (ii) a VL having an amino acid sequence SEQ ID NO:300. In one aspect, provided herein is an antibody that binds Sdc2, wherein the antibody comprises a heavy chain having an amino acid sequence having at least 95% identity to an amino acid sequence of SEQ ID NO:301. In one aspect, provided herein is an antibody that binds Sdc2, wherein the antibody comprises a light chain having an amino acid sequence having at least 95% identity to an amino acid sequence of SEQ ID NO:302. In one aspect, provided herein is an antibody that binds Sdc2, wherein the antibody comprises (i) a heavy chain having an amino acid sequence having at least 95% identity to an amino acid sequence of SEQ ID NO: 301; and (ii) a light chain having an amino acid sequence having at least 95% identity to an amino acid sequence of SEQ ID NO:302. In one aspect, provided herein is an antibody that binds Sdc2, wherein the antibody comprises a heavy chain having an amino acid sequence SEQ ID NO:301. In one aspect, provided herein is an antibody that binds Sdc2, wherein the antibody comprises a light chain having an amino acid sequence SEQ ID NO:302. In one aspect, provided herein is an antibody that binds Sdc2, wherein the antibody comprises (i) a heavy chain having an amino acid sequence SEQ ID NO:301; and (ii) a light chain having an amino acid sequence SEQ ID NO:302.

In one aspect, provided herein is an antibody that binds Sdc2, wherein the antibody that binds to Sdc2 is antibody clone R3-P1-C02. In one aspect, provided herein is an antibody that binds Sdc2, wherein the antibody comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:333; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:334. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 303, 304 and 305, respectively; and (ii) a VL comprising a VL CDR1, a VL CDR2, and a VL CDR3 having an amino acid sequence of SEQ ID NOs: 306, 307 and 308, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 309, 310 and 311, respectively; and (ii) a VL comprising a VL CDR1, a VL CDR2, and a VL CDR3 having an amino acid sequence of SEQ ID NOs: 312, 313 and 314, comprises (i) a VH comprising a VH CDR1, a VH CDR2, and a VH CDR3 having an amino acid sequence of SEQ ID NOs: 315, 316 and 317, respectively; and (ii) a VL comprising a VL CDR1, a VL CDR2, and a VL CDR3 having an amino acid sequence of SEQ ID NOs: 318, 319 and 320, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 321, 322 and 323, respectively; and (ii) a VL comprising a VL CDR1, a VL CDR2, and a VL CDR3 having an amino acid sequence of SEQ ID NOs: 324, 325 and 326, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 327, 328 and 329, respectively; and (ii) a VL comprising a VL CDR1, a VL CDR2, and a VL CDR3 having an amino acid sequence of SEQ ID NOs: 330, 331 and 332, respectively. In one aspect, provided herein is an antibody that binds Sdc2, wherein the antibody comprises a VH having an amino acid sequence having at least 95% identity to an amino acid sequence of SEQ ID NO:333. In one aspect, provided herein is an antibody that binds Sdc2, wherein the antibody comprises a VL having an amino acid sequence having at least 95% identity to an amino acid sequence of SEQ ID NO:334. In one aspect, provided herein is an antibody that binds Sdc2, wherein the antibody comprises (i) a VH having an amino acid sequence having at least 95% identity to an amino acid sequence of SEQ ID NO:333; and (ii) a VL having an amino acid sequence having at least 95% identity to an amino acid sequence of SEQ ID NO:334. In one aspect, provided herein is an antibody that binds Sdc2, wherein the antibody comprises a VH having an amino acid sequence SEQ ID NO:333. In one aspect, provided herein is an antibody that binds Sdc2, wherein the antibody comprises a VL having an amino acid sequence SEQ ID NO:334. In one aspect, provided herein is an antibody that binds Sdc2, wherein the antibody comprises (i) a VH having an amino acid sequence SEQ ID NO: 333; and (ii) a VL having an amino acid sequence SEQ ID NO:334. In one aspect, provided herein is an antibody that binds Sdc2, wherein the antibody comprises a heavy chain having an amino acid sequence having at least 95% identity to an amino acid sequence of SEQ ID NO:335. In one aspect, provided herein is an antibody that binds Sdc2, wherein the antibody comprises a light chain having an amino acid sequence having at least 95% identity to an amino acid sequence of SEQ ID NO:336. In one aspect, provided herein is an antibody that binds Sdc2, wherein the antibody comprises (i) a heavy chain having an amino acid sequence having at least 95% identity to an amino acid sequence of SEQ ID NO: 335; and (ii) a light chain having an amino acid sequence having at least 95% identity to an amino acid sequence of SEQ ID NO:336. In one aspect, provided herein is an antibody that binds Sdc2, wherein the antibody comprises a heavy chain having an amino acid sequence SEQ ID NO:335. In one aspect, amino acid sequence SEQ ID NO:336. In one aspect, provided herein is an antibody that binds Sdc2, wherein the antibody comprises (i) a heavy chain having an amino acid sequence SEQ ID NO:335; and (ii) a light chain having an amino acid sequence SEQ ID NO:336.

In one aspect, provided herein is an antibody that binds Sdc2, wherein the antibody that binds to Sdc2 is antibody clone R3-P3-A12. In one aspect, provided herein is an antibody that binds Sdc2, wherein the antibody comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:367; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:368. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 337, 338 and 339, respectively; and (ii) a VL comprising a VL CDR1, a VL CDR2, and a VL CDR3 having an amino acid sequence of SEQ ID NOs: 340, 341 and 342, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 343, 344 and 345, respectively; and (ii) a VL comprising a VL CDR1, a VL CDR2, and a VL CDR3 having an amino acid sequence of SEQ ID NOs: 346, 347 and 348, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 349, 350 and 351, respectively; and (ii) a VL comprising a VL CDR1, a VL CDR2, and a VL CDR3 having an amino acid sequence of SEQ ID NOs: 352, 353 and 354, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 355, 356 and 357, respectively; and (ii) a VL comprising a VL CDR1, a VL CDR2, and a VL CDR3 having an amino acid sequence of SEQ ID NOs: 358, 359 and 360, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 361, 362 and 363, respectively; and (ii) a VL comprising a VL CDR1, a VL CDR2, and a VL CDR3 having an amino acid sequence of SEQ ID NOs: 364, 365 and 366, respectively. In one aspect, provided herein is an antibody that binds Sdc2, wherein the antibody comprises a VH having an amino acid sequence having at least 95% identity to an amino acid sequence of SEQ ID NO:367. In one aspect, provided herein is an antibody that binds Sdc2, wherein the antibody comprises a VL having an amino acid sequence having at least 95% identity to an amino acid sequence of SEQ ID NO:368. In one aspect, provided herein is an antibody that identity to an amino acid sequence of SEQ ID NO:367; and (ii) a VL having an amino acid sequence having at least 95% identity to an amino acid sequence of SEQ ID NO:368. In one aspect, provided herein is an antibody that binds Sdc2, wherein the antibody comprises a VH having an amino acid sequence SEQ ID NO:367. In one aspect, provided herein is an antibody that binds Sdc2, wherein the antibody comprises a VL having an amino acid sequence SEQ ID NO:368. In one aspect, provided herein is an antibody that binds Sdc2, wherein the antibody comprises (i) a VH having an amino acid sequence SEQ ID NO: 367; and (ii) a VL having an amino acid sequence SEQ ID NO:368. In one aspect, provided herein is an antibody that binds Sdc2, wherein the antibody comprises a heavy chain having an amino acid sequence having at least 95% identity to an amino acid sequence of SEQ ID NO:369. In one aspect, provided herein is an antibody that binds Sdc2, wherein the antibody comprises a light chain having an amino acid sequence having at least 95% identity to an amino acid sequence of SEQ ID NO:370. In one aspect, provided herein is an antibody that binds Sdc2, wherein the antibody comprises (i) a heavy chain having an amino acid sequence having at least 95% identity to an amino acid sequence of SEQ ID NO: 369; and (ii) a light chain having an amino acid sequence having at least 95% identity to an amino acid sequence of SEQ ID NO:370. In one aspect, provided herein is an antibody that binds Sdc2, wherein the antibody comprises a heavy chain having an amino acid sequence SEQ ID NO:369. In one aspect, provided herein is an antibody that binds Sdc2, wherein the antibody comprises a light chain having an amino acid sequence SEQ ID NO:370. In one aspect, provided herein is an antibody that binds Sdc2, wherein the antibody comprises (i) a heavy chain having an amino acid sequence SEQ ID NO:369; and (ii) a light chain having an amino acid sequence SEQ ID NO:370.

In one aspect, provided herein is an antibody that binds Sdc2, wherein the antibody that binds to Sdc2 is antibody clone R4M-P3-A12. In one aspect, provided herein is an antibody that binds Sdc2, wherein the antibody comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:401; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:402. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 371, 372 and 373, respectively; and (ii) a VL comprising a VL CDR1, a VL CDR2, and a VL CDR3 having an amino acid sequence of SEQ ID NOs: 374, 375 and 376, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 377, 378 and 379, respectively; and (ii) a VL comprising a VL CDR1, a VL CDR2, and a VL CDR3 having an amino acid sequence of SEQ ID NOs: 380, 381 and 382, comprises (i) a VH comprising a VH CDR1, a VH CDR2, and a VH CDR3 having an amino acid sequence of SEQ ID NOs: 383, 384 and 385, respectively; and (ii) a VL comprising a VL CDR1, a VL CDR2, and a VL CDR3 having an amino acid sequence of SEQ ID NOs: 386, 387 and 388, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 389, 390 and 391, respectively; and (ii) a VL comprising a VL CDR1, a VL CDR2, and a VL CDR3 having an amino acid sequence of SEQ ID NOs: 392, 393 and 394, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 395, 396 and 397, respectively; and (ii) a VL comprising a VL CDR1, a VL CDR2, and a VL CDR3 having an amino acid sequence of SEQ ID NOs: 398, 399 and 400, respectively. In one aspect, provided herein is an antibody that binds Sdc2, wherein the antibody comprises a VH having an amino acid sequence having at least 95% identity to an amino acid sequence of SEQ ID NO:401. In one aspect, provided herein is an antibody that binds Sdc2, wherein the antibody comprises a VL having an amino acid sequence having at least 95% identity to an amino acid sequence of SEQ ID NO:402. In one aspect, provided herein is an antibody that binds Sdc2, wherein the antibody comprises (i) a VH having an amino acid sequence having at least 95% identity to an amino acid sequence of SEQ ID NO:401; and (ii) a VL having an amino acid sequence having at least 95% identity to an amino acid sequence of SEQ ID NO:402. In one aspect, provided herein is an antibody that binds Sdc2, wherein the antibody comprises a VH having an amino acid sequence SEQ ID NO:401. In one aspect, provided herein is an antibody that binds Sdc2, wherein the antibody comprises a VL having an amino acid sequence SEQ ID NO:402. In one aspect, provided herein is an antibody that binds Sdc2, wherein the antibody comprises (i) a VH having an amino acid sequence SEQ ID NO: 401; and (ii) a VL having an amino acid sequence SEQ ID NO:402. In one aspect, provided herein is an antibody that binds Sdc2, wherein the antibody comprises a heavy chain having an amino acid sequence having at least 95% identity to an amino acid sequence of SEQ ID NO:403. In one aspect, provided herein is an antibody that binds Sdc2, wherein the antibody comprises a light chain having an amino acid sequence having at least 95% identity to an amino acid sequence of SEQ ID NO:404. In one aspect, provided herein is an antibody that binds Sdc2, wherein the antibody comprises (i) a heavy chain having an amino acid sequence having at least 95% identity to an amino acid sequence of SEQ ID NO: 403; and (ii) a light chain having an amino acid sequence having at least 95% identity to an amino acid sequence of SEQ ID NO:404. In one aspect, provided herein is an antibody that binds Sdc2, wherein the antibody comprises a heavy chain having an amino acid sequence SEQ ID NO:403. In one aspect, provided herein is an antibody that binds Sdc2, wherein the antibody comprises a light chain having an amino acid sequence SEQ ID NO:404. In one aspect, provided herein is an antibody that binds Sdc2, wherein the antibody comprises (i) a heavy chain having an amino acid sequence SEQ ID NO:403; and (ii) a light chain having an amino acid sequence SEQ ID NO:404.

In one aspect, provided herein is an antibody that binds Sdc2, wherein the antibody that binds to Sdc2 is antibody clone R4M-P1-A10. In one aspect, provided herein is an antibody that binds Sdc2, wherein the antibody comprises (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:435; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:436. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 405, 406 and 407, respectively; and (ii) a VL comprising a VL CDR1, a VL CDR2, and a VL CDR3 having an amino acid sequence of SEQ ID NOs: 408, 409 and 410, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 411, 412 and 413, respectively; and (ii) a VL comprising a VL CDR1, a VL CDR2, and a VL CDR3 having an amino acid sequence of SEQ ID NOs: 414, 415 and 416, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 417, 418 and 419, respectively; and (ii) a VL comprising a VL CDR1, a VL CDR2, and a VL CDR3 having an amino acid sequence of SEQ ID NOs: 420, 421 and 422, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 423, 424 and 425, respectively; and (ii) a VL comprising a VL CDR1, a VL CDR2, and a VL CDR3 having an amino acid sequence of SEQ ID NOs: 426, 427 and 428, respectively. In one aspect, provided herein is an antibody that binds Sdc2, wherein the antibody comprises (i) a VH comprising a VH CDR1, a VH CDR2, and a VH CDR3 having an amino acid sequence of SEQ ID NOs: 429, 430 and 431, respectively; and (ii) a VL comprising a VL CDR1, a VL CDR2, and a VL CDR3 having an amino acid sequence of SEQ ID NOs: 432, 433 and 434, respectively. In one aspect, provided herein is an antibody that binds Sdc2, wherein the antibody comprises a VH having an amino acid sequence having at least 95% identity to an amino acid sequence of SEQ ID NO:435. In one aspect, provided herein is an antibody that binds Sdc2, wherein the antibody comprises a VL having an amino acid sequence having at least 95% identity to an amino acid sequence of SEQ ID NO:436. In one aspect, provided herein is an antibody that identity to an amino acid sequence of SEQ ID NO:435; and (ii) a VL having an amino acid sequence having at least 95% identity to an amino acid sequence of SEQ ID NO:436. In one aspect, provided herein is an antibody that binds Sdc2, wherein the antibody comprises a VH having an amino acid sequence SEQ ID NO:435. In one aspect, provided herein is an antibody that binds Sdc2, wherein the antibody comprises a VL having an amino acid sequence SEQ ID NO:436. In one aspect, provided herein is an antibody that binds Sdc2, wherein the antibody comprises (i) a VH having an amino acid sequence SEQ ID NO: 435; and (ii) a VL having an amino acid sequence SEQ ID NO:436. In one aspect, provided herein is an antibody that binds Sdc2, wherein the antibody comprises a heavy chain having an amino acid sequence having at least 95% identity to an amino acid sequence of SEQ ID NO:437. In one aspect, provided herein is an antibody that binds Sdc2, wherein the antibody comprises a light chain having an amino acid sequence having at least 95% identity to an amino acid sequence of SEQ ID NO:438. In one aspect, provided herein is an antibody that binds Sdc2, wherein the antibody comprises (i) a heavy chain having an amino acid sequence having at least 95% identity to an amino acid sequence of SEQ ID NO: 437; and (ii) a light chain having an amino acid sequence having at least 95% identity to an amino acid sequence of SEQ ID NO:438. In one aspect, provided herein is an antibody that binds Sdc2, wherein the antibody comprises a heavy chain having an amino acid sequence SEQ ID NO:437. In one aspect, provided herein is an antibody that binds Sdc2, wherein the antibody comprises a light chain having an amino acid sequence SEQ ID NO:438. In one aspect, provided herein is an antibody that binds Sdc2, wherein the antibody comprises (i) a heavy chain having an amino acid sequence SEQ ID NO:437; and (ii) a light chain having an amino acid sequence SEQ ID NO:438.

In some embodiments, the VH CDR1, VH CDR2, VH CDR3, VL CDR1, VL CDR2, and VL CDR3 amino acid sequences are according to the Kabat numbering system. In other embodiments, the VH CDR1, VH CDR2, VH CDR3, VL CDR1, VL CDR2, and VL CDR3 amino acid sequences are according to the Chothia numbering system. In some embodiments, the VH CDR1, VH CDR2, VH CDR3, VL CDR1, VL CDR2, and VL CDR3 amino acid sequences are according to the AbM numbering system; In other embodiments, the VH CDR1, VH CDR2, VH CDR3, VL CDR1, VL CDR2, and VL CDR3 amino acid sequences are according to the Contact numbering system. In some embodiments, the VH CDR1, VH CDR2, VH CDR3, VL CDR1, VL CDR2, and VL CDR3 amino acid sequences are according to the IMGT numbering system.

In one embodiment, the antibody is a humanized antibody. In another embodiment, the antibody is a fully human antibody. In one embodiment, the antibody is an IgG antibody. In one embodiment, the IgG antibody is an IgG1, IgG2, IgG3, or IgG4 antibody. In one embodiment, the antibody comprises a kappa light chain. In one embodiment, the antibody comprises a lambda light chain. In one embodiment, the antibody is a monoclonal antibody. In one embodiment, the antibody is multivalent. In one embodiment, the antibody is a multispecific antibody. In one embodiment, the antibody specifically binds to Sdc2. In one embodiment, the Sdc2 is present on the surface of an endothelial cell. In one embodiment, the Sdc2 is present on the surface of a neural cell. In one embodiment, the Sdc2 is present on the surface of a neuronal cell.

In another aspect, provided herein is an antibody that competes for binding to Sdc2 with any of the anti-Sdc2 antibodies described herein. In another aspect, provided herein is an antibody that binds to the same epitope as any of the anti-Sdc2 antibodies described herein. In another aspect, provided is an anti-Sdc2 antibody that binds an epitope on anti-Sdc2 that overlaps with the epitope on anti-Sdc2 bound by an anti-Sdc2 antibody described herein.

In one aspect, provided is an antibody that competes for binding to anti-Sdc2 with an anti-Sdc2 reference antibody. In another aspect, provided is an anti-Sdc2 antibody that binds to the same Sdc2 epitope as an anti-Sdc2 reference antibody. In another aspect, provided is an anti-Sdc2 antibody that binds an epitope on Sdc2 that overlaps with the epitope on Sdc2 bound by an anti-Sdc2 reference antibody described herein. In another aspect, provided herein is an antibody that competes for binding to Sdc2 with any of the anti-Sdc2 antibodies described herein. In another aspect, provided herein is an antibody that binds to the same epitope as any of the anti-Sdc2 antibodies described herein. In another aspect, provided is an anti-Sdc2 antibody that binds an epitope on Sdc2 that overlaps with the epitope on Sdc2 bound by an anti-Sdc2 antibody described herein. In some embodiments, the anti-Sdc2 antibody comprises a VH CDR1, VH CDR2, and VH CDR3 of an anti-Sdc2 antibody provided herein. In some embodiments, the anti-Sdc2 antibody comprises a VL CDR1, VL CDR2, and VL CDR3 of an anti-Sdc2 antibody provided herein. In some embodiments, the anti-Sdc2 antibody comprises a VH CDR1, VH CDR2, VH CDR3, a VL CDR1, VL CDR2, and VL CDR3 of an anti-Sdc2 antibody provided herein. In some embodiments, the anti-Sdc2 antibody comprises a VH of an anti-Sdc2 antibody provided herein. In some embodiments, the anti-Sdc2 antibody comprises a VL of an anti-Sdc2 antibody provided herein. In some embodiments, the anti-Sdc2 antibody comprises a VH and a VL of an anti-Sdc2 antibody provided herein. In some embodiments, the anti-Sdc2 antibody comprises a VH CDR1, VH CDR2, VH CDR3, a VL CDR1, VL CDR2, and VL CDR3 of an anti-Sdc2 antibody provided herein. In some embodiments, the VH CDR1, VH CDR2, VH CDR3, VL CDR1, VL CDR2, and VL CDR3 amino acid sequences of the anti-Sdc2 antibody are according to the Kabat numbering system. In some embodiments, the VH CDR1, VH CDR2, VH CDR3, VL CDR1, VL CDR2, and VL CDR3 amino acid sequences of the anti-Sdc2 antibody are according to the Chatchai numbering system. In some embodiments, the VH CDR1, VH CDR2, VH CDR3, VL CDR1, VL CDR2, and VL CDR3 amino acid sequences of the anti-Sdc2 antibody are according to the AbM numbering system. In some embodiments, the VH CDR1, VH CDR2, VH CDR3, VL CDR1, VL CDR2, and VL CDR3 amino acid sequences of the anti-Sdc2 antibody are according to the Contact numbering system. In some embodiments, the VH CDR1, VH CDR2, VH CDR3, VL CDR1, VL CDR2, and VL CDR3 amino acid sequences of the anti-Sdc2 antibody are according to the IMGT numbering system.

In another aspect, provided is an antibody that competes for binding to Sdc2 with an anti-Sdc2 reference antibody. In another aspect, provided is an anti-Sdc2 antibody that binds to the same Sdc2 epitope as an anti-Sdc2 reference antibody. In another aspect, provided is an anti-Sdc2 antibody that binds an epitope on Sdc2 that overlaps with the epitope on Sdc2 bound by an anti-Sdc2 reference antibody. In some embodiments, the anti-Sdc2 reference antibody comprises a VH CDR1, VH CDR2, and VH CDR3 of an anti-Sdc2 reference antibody provided herein. In some embodiments, the anti-Sdc2 reference antibody comprises a VL CDR1, VL CDR2, and VL CDR3 of an anti-Sdc2 reference antibody provided herein. In some embodiments, the anti-Sdc2 reference antibody comprises a VH CDR1, VH CDR2, VH CDR3, a VL CDR1, VL CDR2, and VL CDR3 of an anti-Sdc2 reference antibody provided herein. In some embodiments, the anti-Sdc2 reference antibody comprises a VH of an anti-Sdc2 reference antibody provided herein. In some embodiments, the anti-Sdc2 reference antibody comprises a VL of an anti-Sdc2 reference antibody provided herein. In some embodiments, the anti-Sdc2 reference antibody comprises a VH and a VL of an anti-Sdc2 reference antibody provided herein. In some embodiments, the anti-Sdc2 reference antibody comprises a VH CDR1, VH CDR2, VH CDR3, a VL CDR1, VL CDR2, and VL CDR3 of an anti-Sdc2 reference antibody provided herein. In some embodiments, the VH CDR1, VH CDR2, VH CDR3, VL CDR1, VL CDR2, and VL CDR3 amino acid sequences of the anti-Sdc2 reference antibody are according to the Kabat numbering system. In some embodiments, the VH CDR1, VH CDR2, VH CDR3, VL CDR1, VL CDR2, and VL CDR3 amino acid sequences of the anti-Sdc2 reference antibody are according to the Chothia numbering system. In some embodiments, the VH CDR1, VH CDR2, VH CDR3, VL CDR1, VL CDR2, and VL CDR3 amino acid sequences of the anti-Sdc2 reference antibody are according to the AbM numbering system. In some embodiments, the VH CDR1, VH CDR2, VH CDR3, VL CDR1, VL CDR2, and VL CDR3 amino acid sequences of the anti-Sdc2 reference antibody are according to the Contact numbering system. In some embodiments, the VH CDR1, VH CDR2, VH CDR3, VL CDR1, VL CDR2, and VL CDR3 amino acid sequences of the anti-Sdc2 reference antibody are according to the IMGT numbering system.

In some embodiments, an anti-Sdc2 antibody provided herein is chimeric. In some embodiments, an anti-Sdc2 antibody provided herein is human. In some embodiments, an anti-Sdc2 antibody provided herein is humanized. In certain embodiments, an anti-Sdc2 antibody provided herein is an isolated anti-Sdc2 antibody. In some embodiments, a Sdc2 antigen binding fragment provided herein is chimeric. In some embodiments, a Sdc2 antigen binding fragment provided herein is human. In some embodiments, a Sdc2 antigen binding fragment provided herein is humanized. In certain embodiments, a Sdc2 antigen binding fragment provided herein is an isolated Sdc2 antigen binding fragment. In some embodiments, an anti-Sdc2 antibody provided herein is an IgG antibody. In some embodiments, the IgG antibody is an IgG1 antibody. In some embodiments, the IgG antibody is an IgG2 antibody. In some embodiments, the IgG antibody is an IgG3 antibody. In some embodiments, the IgG antibody is an IgG4 antibody. In some embodiments, an anti-Sdc2 antibody provided herein is multivalent. In some embodiments, the anti-Sdc2 antibody is capable of binding at least three antigens. In some embodiments, the anti-Sdc2 antibody is capable of binding at least four antigens. In some embodiments, the anti-Sdc2 antibody is capable of binding at least five antigens.

In another aspect, antibodies or antigen-binding fragments thereof provided herein bind to a region, including an epitope, of Sdc2. For example, in some embodiments, an antibody provided herein binds to a region of human Sdc2 (SEQ ID NO:25) comprising amino acid residues 123 to 140 of human Sdc2. In still another aspect, antibodies provided herein bind to a specific epitope of human Sdc2. For example, in some embodiments, an antibody provided herein binds to a region of mouse Sdc2 (SEQ ID NO: 26) comprising amino acid residues 123 to 140 of mouse Sdc2. In still another aspect, antibodies provided herein bind to a specific epitope of mouse Sdc2.

As used herein, a residue in a first Sdc2 polypeptide (e.g., a human Sdc2 polypeptide or a fragment thereof, or a non-human Sdc2 polypeptide or a fragment thereof) that “corresponds” to a reference residue in a second Sdc2 polypeptide (e.g., a human Sdc2 polypeptide or SEQ ID NO:25) means after aligning the amino acid sequences of the first and second Sdc2 polypeptides, the residue of the first Sdc2 polypeptide that aligns at the same position as the reference residue of the second Sdc2 polypeptide. In some embodiments, the first Sdc2 sequence and the second Sdc2 sequence share at least 50%, at least 60%, at least 70%, at least 80%, at least 90%, at least 95%, or at least 97% sequence identity as determined by the alignment. In some embodiments, the first Sdc2 sequence aligns with a fragment of the second Sdc2 sequence, and the sequence identity between the first Sdc2 sequence and the aligned portion of the second Sdc2 sequence is at least 50%, at least 60%, at least 70%, at least 80%, at least 90%, at least 95%, or at least 97%. In some embodiments, the first Sdc2 sequence is human Sdc2, or a fragment or variant thereof, and the second Sdc2 sequence is human Sdc2 (e.g., SEQ ID NO:25). In alternative embodiments, the first Sdc2 sequence is a non-human vertebrate Sdc2 or a fragment or variant thereof, and the second Sdc2 sequence is human Sdc2 (e.g., SEQ ID NO:25).

In certain embodiments, the antibody or antigen-binding fragment thereof, when binds to a Sdc2 polypeptide, binds to at least one residue in the Sdc2 polypeptide that corresponds to at least one residue in human Sdc2 polypeptide having an amino acid sequence of SEQ ID NO:25. In certain embodiments, the antibody or antigen-binding fragment thereof, when bound to Sdc2, binds to at least one of the residues that correspond to residues 123 to 140 (SEQ ID NO:28) of human Sdc2 (SEQ ID NO:25). In some embodiments, the antibody or antigen-binding fragment thereof, when bound to Sdc2, binds to at least one of the residues that correspond to residues 130 to 137 of human Sdc2 (SEQ ID NO:25). In particular embodiments, the antibody or antigen-binding fragment thereof, when bound to Sdc2, binds to at least one residue that corresponds to the residue selected from the group consisting of P123, A124, E125, E126, D127, T128, N129, V130, Y131, T132, E133, K134, H135, S136, D137, S138, L139, and F140 of human Sdc2 (SEQ ID NO:25).

In particular embodiments, the antibody or antigen-binding fragment thereof, when bound to Sdc2, binds to at least two residues selected from the group of residues that correspond to P123, A124, E125, E126, D127, T128, N129, V130, Y131, T132, E133, K134, H135, S136, D137, S138, L139, and F140 of human Sdc2 (SEQ ID NO:25), respectively. In particular embodiments, the antibody or antigen-binding fragment thereof, when bound to Sdc2, binds to three or more residues selected from the group of residues that correspond to P123, A124, E125, E126, D127, T128, N129, V130, Y131, T132, E133, K134, H135, S136, D137, S138, L139, and F140 of human Sdc2 (SEQ ID NO:25), respectively. In particular embodiments, the antibody or antigen-binding fragment thereof, when bound to Sdc2, binds to four or more residues selected from the group of residues that correspond to P123, A124, E125, E126, D127, T128, N129, V130, Y131, T132, E133, K134, H135, S136, D137, S138, L139, and F140 of human Sdc2 (SEQ ID NO:25), respectively. In particular embodiments, the antibody or antigen-binding fragment thereof, when bound to Sdc2, binds to five or more residues selected from the group of residues that correspond to P123, A124, E125, E126, D127, T128, N129, V130, Y131, T132, E133, K134, H135, S136, D137, S138, L139, and F140 of human Sdc2 (SEQ ID NO:25), respectively. In particular embodiments, the antibody or antigen-binding fragment thereof, when bound to Sdc2, binds to six or more residues selected from the group of residues that correspond to P123, A124, E125, E126, D127, T128, N129, V130, Y131, T132, E133, K134, H135, S136, D137, S138, L139, and F140 of human Sdc2 (SEQ ID NO:25), respectively. In particular embodiments, the antibody or antigen-binding fragment thereof, when bound to Sdc2, binds to seven or more residues selected from the group of residues that correspond to P123, A124, E125, E126, D127, T128, N129, V130, Y131, T132, E133, K134, H135, S136, D137, S138, L139, and F140 of human Sdc2 (SEQ ID NO:25), respectively. In particular embodiments, the antibody or antigen-binding fragment thereof, when bound to Sdc2, binds to eight or more residues selected from the group of residues that correspond to P123, A124, E125, E126, D127, T128, N129, V130, Y131, T132, E133, K134, H135, S136, D137, S138, L139, and F140 of human Sdc2 (SEQ ID NO:25), respectively. In particular embodiments, the antibody or antigen-binding fragment thereof, when bound to Sdc2, binds to nine or more residues selected from the group of residues that correspond to P123, A124, E125, E126, D127, T128, N129, V130, Y131, T132, E133, K134, H135, S136, D137, S138, L139, and F140 of human Sdc2 (SEQ ID NO:25), respectively. In particular embodiments, the antibody or antigen-binding fragment thereof, when bound to Sdc2, binds to ten or more residues selected from the group of residues that correspond to P123, A124, E125, E126, D127, T128, N129, V130, Y131, T132, E133, K134, H135, S136, D137, S138, L139, and F140 of human Sdc2 (SEQ ID NO:25), respectively. In particular embodiments, the antibody or antigen-binding fragment thereof, when bound to Sdc2, binds to eleven or more residues selected from the group of residues that correspond to P123, A124, E125, E126, D127, T128, N129, V130, Y131, T132, E133, K134, H135, S136, D137, S138, L139, and F140 of human Sdc2 (SEQ ID NO:25), respectively. In particular embodiments, the antibody or antigen-binding fragment thereof, when bound to Sdc2, binds to twelve or more residues selected from the group of residues that correspond to P123, A124, E125, E126, D127, T128, N129, V130, Y131, T132, E133, K134, H135, S136, D137, S138, L139, and F140 of human Sdc2 (SEQ ID NO:25), respectively. In particular embodiments, the antibody or antigen-binding fragment thereof, when bound to Sdc2, binds to thirteen or more residues selected from the group of residues that correspond to P123, A124, E125, E126, D127, T128, N129, V130, Y131, T132, E133, K134, H135, S136, D137, S138, L139, and F140 of human Sdc2 (SEQ ID NO:25), respectively. In particular embodiments, the antibody or antigen-binding fragment thereof, when bound to Sdc2, binds to fourteen or more residues selected from the group of residues that correspond to P123, A124, E125, E126, D127, T128, N129, V130, Y131, T132, E133, K134, H135, S136, D137, S138, L139, and F140 of human Sdc2 (SEQ ID NO:25), respectively. In particular embodiments, the antibody or antigen-binding fragment thereof, when bound to Sdc2, binds to fifteen or more residues selected from the group of residues that correspond to P123, A124, E125, E126, D127, T128, N129, V130, Y131, T132, E133, K134, H135, S136, D137, S138, L139, and F140 of human Sdc2 (SEQ ID NO:25), respectively. In particular embodiments, the antibody or antigen-binding fragment thereof, when bound to Sdc2, binds to sixteen or more residues selected from the group of residues that correspond to P123, A124, E125, E126, D127, T128, N129, V130, Y131, T132, E133, K134, H135, S136, D137, S138, L139, and F140 of human Sdc2 (SEQ ID NO:25), respectively. In particular embodiments, the antibody or antigen-binding fragment thereof, when bound to Sdc2, binds to seventeen or more residues selected from the group of residues that correspond to P123, A124, E125, E126, D127, T128, N129, V130, Y131, T132, E133, K134, H135, S136, D137, S138, L139, and F140 of human Sdc2 (SEQ ID NO:25), respectively. In particular embodiments, the antibody or antigen-binding fragment thereof, when bound to Sdc2, binds to all eighteen residues in the group of residues that correspond to P123, A124, E125, E126, D127, T128, N129, V130, Y131, T132, E133, K134, H135, S136, D137, S138, L139, and F140 of human Sdc2 (SEQ ID NO: 25), respectively.

In another embodiment, the antibody or antigen-binding fragment thereof, when bound to Sdc2, binds to a residue that corresponds to P123 within an amino acid sequence of SEQ ID NO:25. In another embodiment, the antibody or antigen-binding fragment thereof, when bound to Sdc2, binds to a residue that corresponds to A124 within an amino acid sequence of SEQ ID NO:25. In another embodiment, the antibody or antigen-binding fragment thereof, when bound to Sdc2, binds to a residue that corresponds to E125 within an amino acid sequence of SEQ ID NO:25. In another embodiment, the antibody or antigen-binding fragment thereof, when bound to Sdc2, binds to a residue that corresponds to E126 within an amino acid sequence of SEQ ID NO:25. In another embodiment, the antibody or antigen-binding fragment thereof, when bound to Sdc2, binds to a residue that corresponds to D127 within an amino acid sequence of SEQ ID NO:25. In another embodiment, the antibody or antigen-binding fragment thereof, when bound to Sdc2, binds to a residue that corresponds to T128 within an amino acid sequence of SEQ ID NO: 25. In another embodiment, the antibody or antigen-binding fragment thereof, when bound to Sdc2, binds to a residue that corresponds to N129 within an amino acid sequence of SEQ ID NO:25. In another embodiment, the antibody or antigen-binding fragment thereof, when bound to Sdc2, binds to a residue that corresponds to V130 within an amino acid sequence of SEQ ID NO:25. In another embodiment, the antibody or antigen-binding fragment thereof, when bound to Sdc2, binds to a residue that corresponds to Y131 within an amino acid sequence of SEQ ID NO:25. In another embodiment, the antibody or antigen-binding fragment thereof, when bound to Sdc2, binds to a residue that corresponds to T132 within an amino acid sequence of SEQ ID NO:25. In another embodiment, the antibody or antigen-binding fragment thereof, when bound to Sdc2, binds to a residue that corresponds to E133 within an amino acid sequence of SEQ ID NO:25. In another embodiment, the antibody or antigen-binding fragment thereof, when bound to Sdc2, binds to a residue that corresponds to K134 within an amino acid sequence of SEQ ID NO:25. In another embodiment, the antibody or antigen-binding fragment thereof, when bound to Sdc2, binds to a residue that corresponds to H135 within an amino acid sequence of SEQ ID NO: 25. In another embodiment, the antibody or antigen-binding fragment thereof, when bound to Sdc2, binds to a residue that corresponds to S136 within an amino acid sequence of SEQ ID NO:25. In another embodiment, the antibody or antigen-binding fragment thereof, when bound to Sdc2, binds to a residue that corresponds to D137 within an amino acid sequence of SEQ ID NO:25. In another embodiment, the antibody or antigen-binding fragment thereof, when bound to Sdc2, binds to a residue that corresponds to S138 within an amino acid sequence of SEQ ID NO:25. In another embodiment, the antibody or antigen-binding fragment thereof, when bound to Sdc2, binds to a residue that corresponds to L139 within an amino acid sequence of SEQ ID NO:25. In another embodiment, the antibody or antigen-binding fragment thereof, when bound to Sdc2, binds to a residue that corresponds to F140 within an amino acid sequence of SEQ ID NO:25. Any combination of two, three, four, five, six, seven, eight, nine, ten or more of the above-referenced amino acid Sdc2 binding sites is also contemplated.

In one aspect, described herein are antibodies that specifically bind to Sdc2 and can modulate Sdc2 activity and/or expression (e.g., activate Sdc2 activity and/or inhibit Sdc2 expression). In certain embodiments, a Sdc2 antagonist is provided herein that is an antibody provided herein that specifically binds to an ECD of Sdc2, and inhibits (e.g., partially inhibits) at least one Sdc2 activity (e.g., decreasing Dep-1 surface expression). In some embodiments, the antibody or antigen-binding fragment thereof, when bound to Sdc2, inhibits binding of Sdc2 to Dep-1. In some embodiments, the antibody or antigen-binding fragment thereof, when bound to Sdc2, blocks binding of Sdc2 to Dep-1. In specific embodiments, antibodies provided herein (e.g., any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, or R4M-P1-A10 or an antigen-binding fragment thereof, or an antibody comprising CDRs of any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, and R4M-P1-A10) specifically bind to Sdc2 and inhibits binding of Sdc2 to Dep-1.

In some embodiments, an anti-Sdc2 antibody or antigen-binding fragment thereof provided herein inhibits binding of Sdc2 to Dep-1 by at least about 10%. In some embodiments, an anti-Sdc2 antibody or antigen-binding fragment thereof provided herein inhibits binding of Sdc2 to Dep-1 by at least about 15%. In some embodiments, an anti-Sdc2 antibody or antigen-binding fragment thereof provided herein inhibits binding of Sdc2 to Dep-1 by at least about 20%. In some embodiments, an anti-Sdc2 antibody or antigen-binding fragment thereof provided herein inhibits binding of Sdc2 to Dep-1 by at least about 25%. In some embodiments, an anti-Sdc2 antibody or antigen-binding fragment thereof provided herein inhibits binding of Sdc2 to Dep-1 by at least about 30%. In some embodiments, an anti-Sdc2 antibody or antigen-binding fragment thereof provided herein inhibits binding of Sdc2 to Dep-1 by at least about 35%. In some embodiments, an anti-Sdc2 antibody or antigen-binding fragment thereof provided herein inhibits binding of Sdc2 to Dep-1 by at least about 40%. In some embodiments, an anti-Sdc2 antibody or antigen-binding fragment thereof provided herein inhibits binding of Sdc2 to Dep-1 by at least about 45%. In some embodiments, an anti-Sdc2 antibody or antigen-binding fragment thereof provided herein inhibits binding of Sdc2 to Dep-1 by at least about 50%. In some embodiments, an anti-Sdc2 antibody or antigen-binding fragment thereof provided herein inhibits binding of Sdc2 to Dep-1 by at least about 55%. In some embodiments, an anti-Sdc2 antibody or antigen-binding fragment thereof provided herein inhibits binding of Sdc2 to Dep-1 by at least about 60%. In some embodiments, an anti-Sdc2 antibody or antigen-binding fragment thereof provided herein inhibits binding of Sdc2 to Dep-1 by at least about 65%. In some embodiments, an anti-Sdc2 antibody or antigen-binding fragment thereof provided herein inhibits binding of Sdc2 to Dep-1 by at least about 70%. In some embodiments, an anti-Sdc2 antibody or antigen-binding fragment thereof provided herein inhibits binding of Sdc2 to Dep-1 by at least about 75%. In some embodiments, an anti-Sdc2 antibody or antigen-binding fragment thereof provided herein inhibits binding of Sdc2 to Dep-1 by at least about 80%. In some embodiments, an anti-Sdc2 antibody or antigen-binding fragment thereof provided herein inhibits binding of Sdc2 to Dep-1 by at least about 85%. In some embodiments, an anti-Sdc2 antibody or antigen-binding fragment thereof provided herein inhibits binding of Sdc2 to Dep-1 by at least about 90%. In some embodiments, an anti-Sdc2 antibody or antigen-binding fragment thereof provided herein inhibits binding of Sdc2 to Dep-1 by at least about 95%. In some embodiments, an anti-Sdc2 antibody or antigen-binding fragment thereof provided herein inhibits binding of Sdc2 to Dep-1 by at least about 98%. In some embodiments, an anti-Sdc2 antibody or antigen-binding fragment thereof provided herein inhibits binding of Sdc2 to Dep-1 by at least about 99%. In some embodiments, an anti-Sdc2 antibody or antigen-binding fragment thereof provided herein inhibits binding of Sdc2 to Dep-1 by at least about 100%.

In some embodiments, the antibody or antigen-binding fragment thereof, when bound to Sdc2, upregulates Dep-1 expression on cell surface. In specific embodiments, antibodies provided herein (e.g., any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, or R4M-P1-A10 or an antigen-binding fragment thereof, or an antibody comprising CDRs of any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, and R4M-P1-A10) specifically bind to Sdc2 and upregulates Dep-1 expression on cell surface. In some embodiments, the cell also expresses Sdc2. In some embodiments, the cell is an endothelial cell.

In some embodiments, an anti-Sdc2 antibody or antigen-binding fragment thereof provided herein increases Dep-1 expression on cell surface by at least about 10%. In some embodiments, an anti-Sdc2 antibody or antigen-binding fragment thereof provided herein increases Dep-1 expression on cell surface by at least about 20%. In some embodiments, an anti-Sdc2 antibody or antigen-binding fragment thereof provided herein increases Dep-1 expression on cell surface by at least about 30%. In some embodiments, an anti-Sdc2 antibody or antigen-binding fragment thereof provided herein increases Dep-1 expression on cell surface by at least about 40%. In some embodiments, an anti-Sdc2 antibody or antigen-binding fragment thereof provided herein increases Dep-1 expression on cell surface by at least about 50%. In some embodiments, an anti-Sdc2 antibody or antigen-binding fragment thereof provided herein increases Dep-1 expression on cell surface by at least about 60%. In some embodiments, an anti-Sdc2 antibody or antigen-binding fragment thereof provided herein increases Dep-1 expression on cell surface by at least about 70%. In some embodiments, an anti-Sdc2 antibody or antigen-binding fragment thereof provided herein increases Dep-1 expression on cell surface by at least about 10%. In some embodiments, an anti-Sdc2 antibody or antigen-binding fragment thereof provided herein increases Dep-1 expression on cell surface by at least about 80%. In some embodiments, an anti-Sdc2 antibody or antigen-binding fragment thereof provided herein increases Dep-1 expression on cell surface by at least about 90%. In some embodiments, an anti-Sdc2 antibody or antigen-binding fragment thereof provided herein increases Dep-1 expression on cell surface by at least about 100%. In some embodiments, an anti-Sdc2 antibody or antigen-binding fragment thereof provided herein increases Dep-1 expression on cell surface by at least about 150%. In some embodiments, an anti-Sdc2 antibody or antigen-binding fragment thereof provided herein increases Dep-1 expression on cell surface by at least about 200%. In some embodiments, an anti-Sdc2 antibody or antigen-binding fragment thereof provided herein increases Dep-1 expression on cell surface by at least about 250%. In some embodiments, an anti-Sdc2 antibody or antigen-binding fragment thereof provided herein increases Dep-1 expression on cell surface by at least about 300%. In some embodiments, an anti-Sdc2 antibody or antigen-binding fragment thereof provided herein increases Dep-1 expression on cell surface by at least about 350%. In some embodiments, an anti-Sdc2 antibody or antigen-binding fragment thereof provided herein increases Dep-1 expression on cell surface by at least about 400%. In some embodiments, an anti-Sdc2 antibody or antigen-binding fragment thereof provided herein increases Dep-1 expression on cell surface by at least about 450%. In some embodiments, an anti-Sdc2 antibody or antigen-binding fragment thereof provided herein increases Dep-1 expression on cell surface by at least about 500%. In some embodiments, an anti-Sdc2 antibody or antigen-binding fragment thereof provided herein increases Dep-1 expression on cell surface by at least about 550%. In some embodiments, an anti-Sdc2 antibody or antigen-binding fragment thereof provided herein increases Dep-1 expression on cell surface by at least about 600%. In some embodiments, an anti-Sdc2 antibody or antigen-binding fragment thereof provided herein increases Dep-1 expression on cell surface by at least about 650%. In some embodiments, an anti-Sdc2 antibody or antigen-binding fragment thereof provided herein increases Dep-1 expression on cell surface by at least about 700%. In some embodiments, an anti-Sdc2 antibody or antigen-binding fragment thereof provided herein increases Dep-1 expression on cell surface by at least about 750%. In some embodiments, an anti-Sdc2 antibody or antigen-binding fragment thereof provided herein increases Dep-1 expression on cell surface by at least about 800%. In some embodiments, an anti-Sdc2 antibody or antigen-binding fragment thereof provided herein increases Dep-1 expression on cell surface by at least about 850%. In some embodiments, an anti-Sdc2 antibody or antigen-binding fragment thereof provided herein increases Dep-1 expression on cell surface by at least about 900%. In some embodiments, an anti-Sdc2 antibody or antigen-binding fragment thereof provided herein increases Dep-1 expression on cell surface by at least about 950%. In some embodiments, an anti-Sdc2 antibody or antigen-binding fragment thereof provided herein increases Dep-1 expression on cell surface by at least about 1000%. Expression levels of Dep-1 can be assessed by methods described herein or known to one of skill in the art (e.g., Western blotting, ELISA, immunohistochemistry, or flow cytometry).

In some embodiments, the antibody or antigen-binding fragment thereof, when bound to Sdc2, reduces endothelial permeability. In specific embodiments, antibodies provided herein (e.g., any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, or R4M-P1-A10 or an antigen-binding fragment thereof, or an antibody comprising CDRs of any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, and R4M-P1-A10) specifically bind to Sdc2 and reduces endothelial permeability.

In some embodiments, an anti-Sdc2 antibody or antigen-binding fragment thereof provided herein reduces endothelial permeability by at least about 10%. In some embodiments, an anti-Sdc2 antibody or antigen-binding fragment thereof provided herein reduces endothelial permeability by at least about 15%. In some embodiments, an anti-Sdc2 antibody or antigen-binding fragment thereof provided herein reduces endothelial permeability by at least about 20%. In some embodiments, an anti-Sdc2 antibody or antigen-binding fragment thereof provided herein reduces endothelial permeability by at least about 25%. In some embodiments, an anti-Sdc2 antibody or antigen-binding fragment thereof provided herein reduces endothelial permeability by at least about 30%. In some embodiments, an anti-Sdc2 antibody or antigen-binding fragment thereof provided herein reduces endothelial permeability by at least about 35%. In some embodiments, an anti-Sdc2 antibody or antigen-binding fragment thereof provided herein reduces endothelial permeability by at least about 40%. In some embodiments, an anti-Sdc2 antibody or antigen-binding fragment thereof provided herein reduces endothelial permeability by at least about 45%. In some embodiments, an anti-Sdc2 antibody or antigen-binding fragment thereof provided herein reduces endothelial permeability by at least about 50%. In some embodiments, an anti-Sdc2 antibody or antigen-binding fragment thereof provided herein reduces endothelial permeability by at least about 55%. In some embodiments, an anti-Sdc2 antibody or antigen-binding fragment thereof provided herein reduces endothelial permeability by at least about 60%. In some embodiments, an anti-Sdc2 antibody or antigen-binding fragment thereof provided herein reduces endothelial permeability by at least about 65%. In some embodiments, an anti-Sdc2 antibody or antigen-binding fragment thereof provided herein reduces endothelial permeability by at least about 70%. In some embodiments, an anti-Sdc2 antibody or antigen-binding fragment thereof provided herein reduces endothelial permeability by at least about 75%. In some embodiments, an anti-Sdc2 antibody or antigen-binding fragment thereof provided herein reduces endothelial permeability by at least about 80%. In some embodiments, an anti-Sdc2 antibody or antigen-binding fragment thereof provided herein reduces endothelial permeability by at least about 85%. In some embodiments, an anti-Sdc2 antibody or antigen-binding fragment thereof provided herein reduces endothelial permeability by at least about 90%. In some embodiments, an anti-Sdc2 antibody or antigen-binding fragment thereof provided herein reduces endothelial permeability by at least about 95%. In some embodiments, an anti-Sdc2 antibody or antigen-binding fragment thereof provided herein reduces endothelial permeability by at least about 98%. In some embodiments, an anti-Sdc2 antibody or antigen-binding fragment thereof provided herein reduces endothelial permeability by at least about 99%. In some embodiments, an anti-Sdc2 antibody or antigen-binding fragment thereof provided herein reduces endothelial permeability by at least about 100%. In some embodiments, the endothelial cell is on a blood vessel. In some embodiments, the blood vessel is a blood vessel in the brain. In some embodiments, the blood vessel is a blood vessel in the eye. In some embodiments, the blood vessel is a blood vessel in the heart. In some embodiments, the endothelial cell is on the fundus of an eye. In some embodiments, the endothelial cell is on a blood vessel in fundus of an eye. In some embodiments, the endothelial cell is on a blood vessel in the retina of an eye. In some embodiments, the endothelial cell is on a blood vessel in the macula of an eye. In some embodiments, the endothelial cell is on a blood vessel in the choroid of an eye. In some embodiments, the endothelial cell is surrounding a nerve cell. In some embodiments, the endothelial cell is in a heart tissue. In some embodiments, the endothelial cell is on a blood vessel in the heart. In some embodiments, the blood vessel is in a heart. In some embodiments, the blood vessel is a heart vein. In some embodiments, the blood vessel is a heart artery. In some embodiments, the blood vessel is part of the left anterior descending coronary artery (LAD). In some embodiments, the endothelial cell expresses Sdc2. In some embodiments, the endothelial permeability is stimulated by contacting the endothelial cells with a VEGF polypeptide. In specific embodiments, the VEGF polypeptide is VEGFA. In specific embodiments, the endothelial permeability is assessed by methods described herein. In some embodiments, the endothelial permeability is assessed by methods known to one of skill in the art. In certain embodiments, the endothelial permeability is relative to endothelial permeability in the presence of stimulation without any anti-Sdc2 antibody. In some embodiments, the endothelial permeability is assessed using a Miles assay. In some embodiments, the endothelial permeability is assessed using a dextran perfusion assay. In specific embodiments, the endothelial permeability is assessed using a FITC-dextran permeability assay.

In some embodiments, the antibody or antigen-binding fragment thereof, when bound to Sdc2, reduces vascular permeability. In specific embodiments, antibodies provided herein (e.g., any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, or R4M-P1-A10 or an antigen-binding fragment thereof, or an antibody comprising CDRs of any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, and R4M-P1-A10) specifically bind to Sdc2 and reduces vascular permeability.

In some embodiments, an anti-Sdc2 antibody or antigen-binding fragment thereof provided herein reduces vascular permeability by at least about 10%. In some embodiments, an anti-Sdc2 antibody or antigen-binding fragment thereof provided herein reduces vascular permeability by at least about 15%. In some embodiments, an anti-Sdc2 antibody or antigen-binding fragment thereof provided herein reduces vascular permeability by at least about 20%. In some embodiments, an anti-Sdc2 antibody or antigen-binding fragment thereof provided herein reduces vascular permeability by at least about 25%. In some embodiments, an anti-Sdc2 antibody or antigen-binding fragment thereof provided herein reduces vascular permeability by at least about 30%. In some embodiments, an anti-Sdc2 antibody or antigen-binding fragment thereof provided herein reduces vascular permeability by at least about 35%. In some embodiments, an anti-Sdc2 antibody or antigen-binding fragment thereof provided herein reduces vascular permeability by at least about 40%. In some embodiments, an anti-Sdc2 antibody or antigen-binding fragment thereof provided herein reduces vascular permeability by at least about 45%. In some embodiments, an anti-Sdc2 antibody or antigen-binding fragment thereof provided herein reduces vascular permeability by at least about 50%. In some embodiments, an anti-Sdc2 antibody or antigen-binding fragment thereof provided herein reduces vascular permeability by at least about 55%. In some embodiments, an anti-Sdc2 antibody or antigen-binding fragment thereof provided herein reduces vascular permeability by at least about 60%. In some embodiments, an anti-Sdc2 antibody or antigen-binding fragment thereof provided herein reduces vascular permeability by at least about 65%. In some embodiments, an anti-Sdc2 antibody or antigen-binding fragment thereof provided herein reduces vascular permeability by at least about 70%. In some embodiments, an anti-Sdc2 antibody or antigen-binding fragment thereof provided herein reduces vascular permeability by at least about 75%. In some embodiments, an anti-Sdc2 antibody or antigen-binding fragment thereof provided herein reduces vascular permeability by at least about 80%. In some embodiments, an anti-Sdc2 antibody or antigen-binding fragment thereof provided herein reduces vascular permeability by at least about 85%. In some embodiments, an anti-Sdc2 antibody or antigen-binding fragment thereof provided herein reduces vascular permeability by at least about 90%. In some embodiments, an anti-Sdc2 antibody or antigen-binding fragment thereof provided herein reduces vascular permeability by at least about 95%. In some embodiments, an anti-Sdc2 antibody or antigen-binding fragment thereof provided herein reduces vascular permeability by at least about 98%. In some embodiments, an anti-Sdc2 antibody or antigen-binding fragment thereof provided herein reduces vascular permeability by at least about 99%. In some embodiments, an anti-Sdc2 antibody or antigen-binding fragment thereof provided herein reduces vascular permeability by at least about 100%. In some embodiments, the vascular permeability is the vascular permeability of a blood vessel. In some embodiments, the blood vessel is a blood vessel in the brain. In some embodiments, the blood vessel is a blood vessel in the eye. In some embodiments, the blood vessel is a blood vessel in the heart. In some embodiments, the blood vessel is on the fundus of an eye. In some embodiments, the blood vessel in the retina of an eye. In some embodiments, the blood vessel in macula of an eye. In some embodiments, the blood vessel is in the choroid of an eye. In some embodiments, the blood vessel is in a heart. In some embodiments, the blood vessel is a heart vein. In some embodiments, the blood vessel is a heart artery. In some embodiments, the blood vessel is part of the left anterior descending coronary artery (LAD). In some embodiments, the vascular permeability is stimulated by a VEGF polypeptide. In specific embodiments, the VEGF polypeptide is VEGFA. In specific embodiments, the vascular permeability is assessed by methods described herein. In some embodiments, the vascular permeability is assessed by methods known to one of skill in the art. In certain embodiments, the vascular permeability is relative to vascular permeability in the presence of stimulation without any anti-Sdc2 antibody. In some embodiments, the vascular permeability is assessed using a Miles assay. In some embodiments, the vascular permeability is assessed using a dextran perfusion assay. In specific embodiments, the vascular permeability is assessed using a FITC-dextran permeability assay.

In some embodiments, the antibody or antigen-binding fragment thereof, when bound to Sdc2, does not impact angiogenesis in a tissue. In specific embodiments, antibodies provided herein (e.g., any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, or R4M-P1-A10 or an antigen-binding fragment thereof, or an antibody comprising CDRs of any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, and R4M-P1-A10) specifically bind to Sdc2 and do not impact angiogenesis.

In some embodiments, the angiogenesis is assessed by measuring expression of one or more endothelial marker proteins in the tissue. In some embodiments, the one or more endothelial marker proteins are selected from CD31 (PECAM1), VWF, CD93, EGFL7, ID3, FLT1, GNG11, MCAM, FLT4, PLVAP, ADGRF5, ABCG2, ACVRL1, NRP2, FHL2, ARHGEF15, EMCN, ADGRL4, PTPRB, CLDN5, ELK3, CDH5, ENG, KDR, EPAS1, ETS1, CD34, CLIC4, LY6A, IGFBP7, ID1, ICAM1, ITGB3, SELE, VCAM1, PROCR, TEK, APLN, NOS3, THBD, ACKR1, SLCO1C1, TMEM100, ABCB1A, PODXL, NOSTRIN, MFSD2A, AQP1, MYLK, RASIP1, FLI1, TIE1, APLNR, ADAMTS1, RPRM, FABP4, LOX, CARD10, CLEC14A, DLL4, ESM1, EXOC3L, GIMAP5, GJA4, MMRN2, NOTCH4, NPR1, PRKCH, RASGRP3, ROBO4, SCARF1, SOX18, SOX7, SPNS2, THSD1, APOLD1, EMP1, CD36, RNASE1, CTGF, HYAL2, CLEC4G, GPR182, F8, RBP7, CALCRL, FOXF1, CASZ1, AQP7, TCF15, CD300LG, BTNL9, MEOX2, ERG, HEXIM1, GLYCAM1, CD55, MMRN1, C7, RAMP3, VEGFC, GJA5, HEY1, RND1, BDP1, CD46, MEOX1, CCL19, CLCA3A1, MADCAM1, CYP1B1, IRX3, BIRC2, LYVE1, SEMA3D, SLCO1A4, WFDC1, VWA1, ECE1, SDPR, CAR4, TBX1, SEMA7A, FOXF2, PDGFB, ECSCR, PLEC, STAB1, TGFBR2, CXCL1, RGS5, SLC7A5, SLC2A1, EDNRB, KCNJ8, CD82, CHST1, PLAC8, TSPAN8, PDPN, PROX1, EHD3, SRGN, S100A10, USHBP1, MYF6, OIT3, IL1A, BMP2, C1QTNF1, PCDH12, DPP4, PALMD, POSTN, BMX, SLC38A5, XDH, SPARC, MGLL, SLC9A3R2, RGCC, ICAM2, MGP, SPARCL1, TM4SF1, ADIRF, CD9, SRPX, CAV1, HSPG2, CCL14, CLEC1B, FCN2, S100A13, FCN3, CRHBP, IFI27, CCL23, SGK1, DNASE1L3, LIFR, PCAT19, CDKN1C, INMT, PTGDS, TIMP3, GPM6A, FAM167B, LTC4S, STAB2, GPIHBP1, KLK1, ADORA2A, ARAP3, and LY6C1. In some embodiments, the tissue expresses Sdc2. In some embodiments, the tissue is in the eye of a subject. In some embodiments, the tissue is in the fundus of the eye. In some embodiments, the tissue is in the retina of the eye. In some embodiments, the tissue is in the macula of the eye. In some embodiments, the tissue is in the choroid of the eye. In some embodiments, the one or more endothelial marker protein comprises ERG. In some embodiments, the antibody or antigen-binding fragment thereof, when bound to Sdc2, expression of ERG remains substantially the same in the tissue. Expression levels of ERG can be assessed by methods described herein or known to one of skill in the art (e.g., Western blotting, ELISA, immunohistochemistry, or flow cytometry). In some embodiments, the tissue is the brain. In other embodiments, the tissue is the heart.

In some embodiments, the antibody or antigen-binding fragment thereof, when bound to Sdc2, reduces edema in a tissue. In specific embodiments, antibodies provided herein (e.g., any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, or R4M-P1-A10 or an antigen-binding fragment thereof, or an antibody comprising CDRs of any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, and R4M-P1-A10) specifically bind to Sdc2 and reduce edema.

In some embodiments, an anti-Sdc2 antibody or antigen-binding fragment thereof provided herein reduces the edema volume by at least about 10%. In some embodiments, an anti-Sdc2 antibody or antigen-binding fragment thereof provided herein reduces the edema volume by at least about 15%. In some embodiments, an anti-Sdc2 antibody or antigen-binding fragment thereof provided herein reduces the edema volume by at least about 20%. In some embodiments, an anti-Sdc2 antibody or antigen-binding fragment thereof provided herein reduces the edema volume by at least about 25%. In some embodiments, an anti-Sdc2 antibody or antigen-binding fragment thereof provided herein reduces the edema volume by at least about 30%. In some embodiments, an anti-Sdc2 antibody or antigen-binding fragment thereof provided herein reduces the edema volume by at least about 35%. In some embodiments, an anti-Sdc2 antibody or antigen-binding fragment thereof provided herein reduces the edema volume by at least about 40%. In some embodiments, an anti-Sdc2 antibody or antigen-binding fragment thereof provided herein reduces the edema volume by at least about 45%. In some embodiments, an anti-Sdc2 antibody or antigen-binding fragment thereof provided herein reduces the edema volume by at least about 50%. In some embodiments, an anti-Sdc2 antibody or antigen-binding fragment thereof provided herein reduces the edema volume by at least about 55%. In some embodiments, an anti-Sdc2 antibody or antigen-binding fragment thereof provided herein reduces the edema volume by at least about 60%. In some embodiments, an anti-Sdc2 antibody or antigen-binding fragment thereof provided herein reduces the edema volume by at least about 65%. In some embodiments, an anti-Sdc2 antibody or antigen-binding fragment thereof provided herein reduces the edema volume by at least about 70%. In some embodiments, an anti-Sdc2 antibody or antigen-binding fragment thereof provided herein reduces the edema volume by at least about 75%. In some embodiments, an anti-Sdc2 antibody or antigen-binding fragment thereof provided herein reduces the edema volume by at least about 80%. In some embodiments, an anti-Sdc2 antibody or antigen-binding fragment thereof provided herein reduces the edema volume by at least about 85%. In some embodiments, an anti-Sdc2 antibody or antigen-binding fragment thereof provided herein reduces the edema volume by at least about 90%. In some embodiments, an anti-Sdc2 antibody or antigen-binding fragment thereof provided herein reduces the edema volume by at least about 95%. In some embodiments, an anti-Sdc2 antibody or antigen-binding fragment thereof provided herein reduces the edema volume by at least about 98%. In some embodiments, an anti-Sdc2 antibody or antigen-binding fragment thereof provided herein reduces the edema volume by at least about 99%. In some embodiments, an anti-Sdc2 antibody or antigen-binding fragment thereof provided herein reduces the edema volume by at least about 100%. In some embodiments, the edema is formed in the brain. In some embodiments, the edema is formed in a heart tissue. In specific embodiments, the edema volume is assessed by methods described herein. In some embodiments, the edema volume is assessed by methods known to one of skill in the art. In some embodiments, the edema is measured by measuring the heart mass. In some embodiments, the edema volume is measured by measuring the left ventricular mass of a heart.

In some embodiments, the antibody or antigen-binding fragment thereof, when bound to Sdc2, reduces inflammation in a tissue. In specific embodiments, antibodies provided herein (e.g., any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, or R4M-P1-A10 or an antigen-binding fragment thereof, or an antibody comprising CDRs of any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, and R4M-P1-A10) specifically bind to Sdc2 and reduce inflammation in the tissue. In some embodiments, the inflammation is acute inflammation. In some embodiments, the inflammation is chronic inflammation.

In some embodiments, the anti-Sdc2 antibody or antigen binding fragment thereof reduces expression of a pro-inflammatory cytokine in the tissue. In some embodiments, the anti-SDC-2 antibody or antigen binding fragment reduces expression of a pro-inflammatory cytokine by at least 10%. In some embodiments, the anti-SDC-2 antibody or antigen binding fragment reduces expression of a pro-inflammatory cytokine by at least 15%. In some embodiments, the anti-SDC-2 antibody or antigen binding fragment reduces expression of a pro-inflammatory cytokine by at least 20%. In some embodiments, the anti-SDC-2 antibody or antigen binding fragment reduces expression of a pro-inflammatory cytokine by at least 25%. In some embodiments, the anti-SDC-2 antibody or antigen binding fragment reduces expression of a pro-inflammatory cytokine by at least 30%. In some embodiments, the anti-SDC-2 antibody or antigen binding fragment reduces expression of a pro-inflammatory cytokine by at least 35%. In some embodiments, the anti-SDC-2 antibody or antigen binding fragment reduces expression of a pro-inflammatory cytokine by at least 40%. In some embodiments, the anti-SDC-2 antibody or antigen binding fragment reduces expression of a pro-inflammatory cytokine by at least 45%. In some embodiments, the anti-SDC-2 antibody or antigen binding fragment reduces expression of a pro-inflammatory cytokine by at least 50%. In some embodiments, the anti-SDC-2 antibody or antigen binding fragment reduces expression of a pro-inflammatory cytokine by at least 55%. In some embodiments, the anti-SDC-2 antibody or antigen binding fragment reduces expression of a pro-inflammatory cytokine by at least 60%. In some embodiments, the anti-SDC-2 antibody or antigen binding fragment reduces expression of a pro-inflammatory cytokine by at least 65%. In some embodiments, the anti-SDC-2 antibody or antigen binding fragment reduces expression of a pro-inflammatory cytokine by at least 70%. In some embodiments, the anti-SDC-2 antibody or antigen binding fragment reduces expression of a pro-inflammatory cytokine by at least 75%. In some embodiments, the anti-SDC-2 antibody or antigen binding fragment reduces expression of a pro-inflammatory cytokine by at least 80%. In some embodiments, the anti-SDC-2 antibody or antigen binding fragment reduces expression of a pro-inflammatory cytokine by at least 85%. In some embodiments, the anti-SDC-2 antibody or antigen binding fragment reduces expression of a pro-inflammatory cytokine by at least 90%. In some embodiments, the anti-SDC-2 antibody or antigen binding fragment reduces expression of a pro-inflammatory cytokine by at least 95%. In some embodiments, the anti-SDC-2 antibody or antigen binding fragment reduces expression of a pro-inflammatory cytokine by at least 98%. In some embodiments, the anti-SDC-2 antibody or antigen binding fragment reduces expression of a pro-inflammatory cytokine by at least 99%. In some embodiments, the anti-SDC-2 antibody or antigen binding fragment reduces expression of a pro-inflammatory cytokine by at least 100%. In some embodiments, the cytokine is selected from the group consisting of IL-1, IL-2, IL-3, IL-6, IL-12, IL-17, IL-22, IL-23, GM-CSF, MIG, CCL11, IFN-γ, TNF-α, and MCP1 (CCL2). In some embodiments, the anti-Sdc2 antibody or antigen binding fragment thereof reduces expression of IL-1 in the tissue. In some embodiments, the anti-Sdc2 antibody or antigen binding fragment thereof reduces expression of IL-2 in the tissue. In some embodiments, the anti-Sdc2 antibody or antigen binding fragment thereof reduces expression of IL-3 in the tissue. In some embodiments, the anti-Sdc2 antibody or antigen binding fragment thereof reduces expression of IL-6 in the tissue. In some embodiments, the anti-Sdc2 antibody or antigen binding fragment thereof reduces expression of IL-12 in the tissue. In some embodiments, the anti-Sdc2 antibody or antigen binding fragment thereof reduces expression of IL-17 in the tissue. In some embodiments, the anti-Sdc2 antibody or antigen binding fragment thereof reduces expression of IL-1 in the tissue. In some embodiments, the anti-Sdc2 antibody or antigen binding fragment thereof reduces expression of IL-22 in the tissue. In some embodiments, the anti-Sdc2 antibody or antigen binding fragment thereof reduces expression of IL-23 in the tissue. In some embodiments, the anti-Sdc2 antibody or antigen binding fragment thereof reduces expression of GM-CSF in the tissue. In some embodiments, the anti-Sdc2 antibody or antigen binding fragment thereof reduces expression of MIG in the tissue. In some embodiments, the anti-Sdc2 antibody or antigen binding fragment thereof reduces expression of CCL11 in the tissue. In some embodiments, the anti-Sdc2 antibody or antigen binding fragment thereof reduces expression of IFN-γ in the tissue. In some embodiments, the anti-Sdc2 antibody or antigen binding fragment thereof reduces expression of TNF-α in the tissue. In some embodiments, the anti-Sdc2 antibody or antigen binding fragment thereof reduces expression of MCP1 (CCL2) in the tissue. In some embodiments, the tissue expresses Sdc2. In some embodiments, the tissue is in the eye of a subject. In some embodiments, the tissue is in the fundus of the eye. In some embodiments, the tissue is in the retina of the eye. In some embodiments, the tissue is in the macula of the eye. In some embodiments, the tissue is in the choroid of the eye. In some embodiments, the tissue expresses Sdc2. In some embodiments, the tissue is in the heart of a subject. In some embodiments, the tissue is in left ventricular of the heart. In other embodiments, the tissue is in the brain of a subject.

In some embodiments, the antibody or antigen-binding fragment thereof, when bound to Sdc2, reduces recruitment of immune cells to the tissue. In specific embodiments, antibodies provided herein (e.g., any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, or R4M-P1-A10 or an antigen-binding fragment thereof, or an antibody comprising CDRs of any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, and R4M-P1-A10) specifically bind to Sdc2 and reduce recruitment of immune cells to the tissue.

In some embodiments, the anti-Sdc2 antibody or antigen-binding fragment thereof reduces recruit of immune cells to the tissue by at least about 10%. In some embodiments, the anti-Sdc2 antibody or antigen-binding fragment thereof reduces recruit of immune cells to the tissue by at least about 15%. In some embodiments, the anti-Sdc2 antibody or antigen-binding fragment thereof reduces recruit of immune cells to the tissue by at least about 20%. In some embodiments, the anti-Sdc2 antibody or antigen-binding fragment thereof reduces recruit of immune cells to the tissue by at least about 25%. In some embodiments, the anti-Sdc2 antibody or antigen-binding fragment thereof reduces recruit of immune cells to the tissue by at least about 30%. In some embodiments, the anti-Sdc2 antibody or antigen-binding fragment thereof reduces recruit of immune cells to the tissue by at least about 35%. In some embodiments, the anti-Sdc2 antibody or antigen-binding fragment thereof reduces recruit of immune cells to the tissue by at least about 40%. In some embodiments, the anti-Sdc2 antibody or antigen-binding fragment thereof reduces recruit of immune cells to the tissue by at least about 45%. In some embodiments, the anti-Sdc2 antibody or antigen-binding fragment thereof reduces recruit of immune cells to the tissue by at least about 50%. In some embodiments, the anti-Sdc2 antibody or antigen-binding fragment thereof reduces recruit of immune cells to the tissue by at least about 55%. In some embodiments, the anti-Sdc2 antibody or antigen-binding fragment thereof reduces recruit of immune cells to the tissue by at least about 60%. In some embodiments, the anti-Sdc2 antibody or antigen-binding fragment thereof reduces recruit of immune cells to the tissue by at least about 65%. In some embodiments, the anti-Sdc2 antibody or antigen-binding fragment thereof reduces recruit of immune cells to the tissue by at least about 70%. In some embodiments, the anti-Sdc2 antibody or antigen-binding fragment thereof reduces recruit of immune cells to the tissue by at least about 75%. In some embodiments, the anti-Sdc2 antibody or antigen-binding fragment thereof reduces recruit of immune cells to the tissue by at least about 80%. In some embodiments, the anti-Sdc2 antibody or antigen-binding fragment thereof reduces recruit of immune cells to the tissue by at least about 85%. In some embodiments, the anti-Sdc2 antibody or antigen-binding fragment thereof reduces recruit of immune cells to the tissue by at least about 90%. In some embodiments, the anti-Sdc2 antibody or antigen-binding fragment thereof reduces recruit of immune cells to the tissue by at least about 95%. In some embodiments, the anti-Sdc2 antibody or antigen-binding fragment thereof reduces recruit of immune cells to the tissue by at least about 98%. In some embodiments, the anti-Sdc2 antibody or antigen-binding fragment thereof reduces recruit of immune cells to the tissue by at least about 99%. In some embodiments, the anti-Sdc2 antibody or antigen-binding fragment thereof reduces recruit of immune cells to the tissue by at least about 100%. In some embodiments, the tissue expresses Sdc2. In some embodiments, the tissue is in the eye of a subject. In some embodiments, the tissue is in the fundus of the eye. In some embodiments, the tissue is in the retina of the eye. In some embodiments, the tissue is in the macula of the eye. In some embodiments, the tissue is in the choroid of the eye. In some embodiments, the tissue expresses Sdc2. In some embodiments, the tissue is in the heart of a subject. In some embodiments, the tissue is in left ventricular of the heart. In other embodiments, the tissue is in the brain of a subject.

In some embodiments, the immune cells are selected from T cells, B cells, natural killer cells, neutrophils, mast cells, macrophages, antigen-presenting cells (APC), basophils, and eosinophils. In some embodiments, reduction of recruitment of immune cells to the tissue is assessed by methods described herein. In some embodiments, reduction of recruitment of immune cells to the tissue is assessed by methods known to one skilled in the art. In some embodiments, reduction of recruitment of immune cells to the tissue is assessed by measuring the amount of a biomarker for a type of immune cells. In specific embodiments, reduction of recruitment of immune cells to the tissue is assessed by the reduction of a T cell marker in the tissue. In some embodiments, the T cell marker is selected from TRBC2, CD3D, CD3G, CD3E, LTB, IL7R, LEF1, GZMK, TRAC, HOPX, IFIT3, CERK, GIMAP3, CXCR6, SATB1, PTPRCAP, CD69, TRBC1, CD2, GDPD3, IL2RA, CCL5, MYB, GZMA, CD52, CCL4, GIMAP2, SYT3, NOTCH3, SEMA6D, DKK3, PMCH, ITK, GEM, MAFF, TGIF1, RORA, TNFAIP3, CREM, PXDC1, NABP1, FAM110A, EEF1B2P3, PFN1P1, IL32, CXCR4, SEPT1, BCL2, CYTL1, CTSW, PTPN22, TXK, TRAF1, CD8B, BATF3, GZMH, LAG3, CD8B1, GZMB, SH2D1A, MYO1G, FMNL1, S1PR4, CD247, MS4A4C, GIMAP5, CD28, CD160, TCRG-C2, TCRG-C1, TCRG-C4, TRDC, H2-T3, H2-T10, RHOH, KLRB1, CCR2, IL2RB, CD163L1, TRDV4, MBD2, ICOS, IL18R1, TNFRSF4, CCL20, CLEC2D, CD8A, CD6, S100A4, LCK, CD81, THY1, LAT, SKAP1, TCF7, CCL4L2, PYHIN1, JUNB, DUSP2, IFNG, BRAF, CCL3, CCL6, CD4, CD7, H2-Q7, and CCR7. In specific embodiments, reduction of recruitment of immune cells to the tissue is assessed by the reduction of a B cell marker in the tissue. In some embodiments, the B cell marker is selected from PXK, CD19, MS4A1, CD74, CD79A, PTPRC, IGHD, IGHM, HLA-DRA, PAX5, BANK1, CR2, CD22, FCER2, CD79B, IGLL1, SPN, B3GAT1, CD72, LY6D, IGLC1, CD5, CD40, CD69, CD70, CD86, TNFRSF9, SDC1, TNFSF4, TNFRSF13B, TNFRSF13C, PDCD1, RASGRP3, HLA-DQA1, FLI1, CD14, SEMA6D, LAIR1, IFIT3, DNTT, CD24, CD27, MUM1, JCHAIN, MZB1, H2-DMB2, FCMR, EDEM1, VPREB3, H2-OB, POU2AF1, CRELD2, DERL3, SIGLECG, RALGPS2, FCHSD2, POLD4, CMAH, TNFRSF17, HVCN1, TRP53INP1, FCRLA, EDEM2, BLNK, H2-OA, TXNDC11, BTLA, SMAP2, SCD1, FAM46C, FKBP11, SEC61A1, SPCS3, SPIB, EAF2, CXCR4, BIRC3, IGLC2, IGLC3, IL21R, IGKC, IGLV1, VPREB1, VPREB2, LRMP, KLHL6, SLAMF6, FAM129C, BST1, MSH5, DOK3, BACH2, FCER2A, IGHG3, IGHG4, IGHA, CD38, EBF1, BCL11A, CCR7, CD55, CD52, TLR9, SWAP70, HMGA1, CD2, CD80, LTB, MME, and IGHG1. In specific embodiments, reduction of recruitment of immune cells to the tissue is assessed by the reduction of a macrophage marker in the tissue. In some embodiments, the macrophage marker is selected from CD11b, CD68, FCGR1, NAAA, JAML, TYROBP, TREM2, H2-DMA, CXCL16, MGL2, CLEC4A2, CCL12, PARP14, SEPP1, MARCH1, FGL2, LYZ2, WFDC17, CPM, CHIT1, TGFBR1, SLAMF9, SCIMP, LILRA5, C3AR1, FGD2, RAB7B, RBPJ, SLCO2B1, EGLN3, CLEC4D, ADAM8, ARL11, MMP12, VSIG4, RETNLA, RAB20, SNX20, FMNL1, GPR132, NCEH1, CCL24, CD300A, CCL7, IRF5, MYO1G, DUSP5, GPR171, SAMSN1, NR4A3, HILPDA, SLC37A2, LPCAT2, IFNAR2, MS4A7, HPGD, CD5L, LILR4B, CYTH4, CLEC4N, CD209F, CSF1R, FGR, CYBB, CD200, CD200R1, GATA6, H2-AB1, H2-EB1, MAF, AIF1, ADGRE5, CLEC4A3, CLEC4A1, SLC15A3, AKR1B3, CYP27A1, ITGAL, ITGAM, CD14, FUT4, FCGR3A, CD33, FCGR1A, CD80, LILRB4, CD86, CD163, CCR5, TLR2, TLR4, ADGRE1 (f4/80), GPR34, CCL9, FABP4, S100A8, F13A1, CD83, STAB1, MRC1, CCL6, GPNMB, LYVE1, PLTP, MS4A4A, MS4A6A, FPR1, CD180, GDF15, HFE, TNF, CCR2, CD209A, C5AR1, CXCL2, CCL2, IL1B, AHR, CCR7, DNASE1L3, CXCL1, CCL22, S100A4, MMP9, NRP2, CTSK, CD36, HPGDS, SLC11A1, CCL3, CLEC7A, CCL5, CD3E, CD19, CD74, LYZ1, LGALS3, UCP2, TREML4, IL4RA, ITGAX, LY6C1, PPARG, SIGLECF, RGS1, DAB2, P2RY6, CLEC10A, ADGRE4, RUNX3, SYK, CX3CR1, SLAMF7, and CD31 (PECAM1).

In specific embodiments, upon administration of the anti-Sdc2 antibody or antigen binding fragment thereof, ADGRE1 expression level in the eye tissue is reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100%. In specific embodiments, upon administration of the anti-Sdc2 antibody or antigen binding fragment thereof, CD31 expression level in the eye tissue is reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100%. In specific embodiments, reduction of recruitment of immune cells to the tissue is assessed by the reduction of a natural killer cell marker in the tissue. In some embodiments, the natural killer cell marker is selected from NCAM1, IL2RB, CD44, IL12RB2, CXCR4, ZFP683, SLAMF7, TCF7, STYK1, S1PR1, GATA3, TBX21, ZBTB16, PRF1, TNFRSF8, GZMB, NR4A1, MAP3K8, EGR1, ITGB2, RORA, KLRB1, IL12RB1, and IL17RA.

In specific embodiments, reduction of recruitment of immune cells to the tissue is assessed by the reduction of a natural killer cell marker in the tissue. In some embodiments, the natural killer cell marker is selected from NCAM1, IL2RB, CD44, IL12RB2, CXCR4, ZFP683, SLAMF7, TCF7, STYK1, S1PR1, GATA3, TBX21, ZBTB16, PRF1, TNFRSF8, GZMB, NR4A1, MAP3K8, EGR1, ITGB2, RORA, KLRB1, IL12RB1, and IL17RA.

In some embodiments, the antibody or antigen-binding fragment thereof, when bound to Sdc2, reduces the infarct size in the heart of a subject suffered from AMI. In specific embodiments, antibodies provided herein (e.g., any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, or R4M-P1-A10 or an antigen-binding fragment thereof, or an antibody comprising CDRs of any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, and R4M-P1-A10) specifically bind to Sdc2 and reduce infarct size.

In some embodiments, an anti-Sdc2 antibody or antigen-binding fragment thereof provided herein reduces the infarct size by at least about 10%. In some embodiments, an anti-Sdc2 antibody or antigen-binding fragment thereof provided herein reduces the infarct size by at least about 15%. In some embodiments, an anti-Sdc2 antibody or antigen-binding fragment thereof provided herein reduces the infarct size by at least about 20%. In some embodiments, an anti-Sdc2 antibody or antigen-binding fragment thereof provided herein reduces the infarct size by at least about 25%. In some embodiments, an anti-Sdc2 antibody or antigen-binding fragment thereof provided herein reduces the infarct size by at least about 30%. In some embodiments, an anti-Sdc2 antibody or antigen-binding fragment thereof provided herein reduces the infarct size by at least about 35%. In some embodiments, an anti-Sdc2 antibody or antigen-binding fragment thereof provided herein reduces the infarct size by at least about 40%. In some embodiments, an anti-Sdc2 antibody or antigen-binding fragment thereof provided herein reduces the infarct size by at least about 45%. In some embodiments, an anti-Sdc2 antibody or antigen-binding fragment thereof provided herein reduces the infarct size by at least about 50%. In some embodiments, an anti-Sdc2 antibody or antigen-binding fragment thereof provided herein reduces the infarct size by at least about 55%. In some embodiments, an anti-Sdc2 antibody or antigen-binding fragment thereof provided herein reduces the infarct size by at least about 60%. In some embodiments, an anti-Sdc2 antibody or antigen-binding fragment thereof provided herein reduces the infarct size by at least about 65%. In some embodiments, an anti-Sdc2 antibody or antigen-binding fragment thereof provided herein reduces the infarct size by at least about 70%. In some embodiments, an anti-Sdc2 antibody or antigen-binding fragment thereof provided herein reduces the infarct size by at least about 75%. In some embodiments, an anti-Sdc2 antibody or antigen-binding fragment thereof provided herein reduces the infarct size by at least about 80%. In some embodiments, an anti-Sdc2 antibody or antigen-binding fragment thereof provided herein reduces the infarct size by at least about 85%. In some embodiments, an anti-Sdc2 antibody or antigen-binding fragment thereof provided herein reduces the infarct size by at least about 90%. In some embodiments, an anti-Sdc2 antibody or antigen-binding fragment thereof provided herein reduces the infarct size by at least about 95%. In some embodiments, an anti-Sdc2 antibody or antigen-binding fragment thereof provided herein reduces the infarct size by at least about 98%. In some embodiments, an anti-Sdc2 antibody or antigen-binding fragment thereof provided herein reduces the infarct size by at least about 99%. In some embodiments, an anti-Sdc2 antibody or antigen-binding fragment thereof provided herein reduces the infarct size by at least about 100%. In some embodiments, the infarct is in the left ventricular of the heart. In some embodiments, the infarct is in the right atrium of the heart. In specific embodiments, the infarct size is assessed by methods described herein. In some embodiments, the infarct size is assessed by methods known to one of skill in the art. In some embodiments, the infarct size is assessed using a 2,3,5-Triphenyltetrazolium chloride (TTC) staining. In some embodiments, the infarct size is assessed using a Masson's trichrome staining. In some embodiments, the infarct size is measured by a heart imaging technique known in the art. In some embodiments, the infarct size is assessed using echocardiography following standards of the art.

In some embodiments, the antibody or antigen-binding fragment thereof, when bound to Sdc2, increases the left ventricular (LV) function (LVEF) of a subject. In specific embodiments, antibodies provided herein (e.g., any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, or R4M-P1-A10 or an antigen-binding fragment thereof, or an antibody comprising CDRs of any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, and R4M-P1-A10) specifically bind to Sdc2 and enhance the subject's LVEF.

In some embodiments, an anti-Sdc2 antibody or antigen-binding fragment thereof provided herein enhances the subject's LVEF by at least about 10%. In some embodiments, an anti-Sdc2 antibody or antigen-binding fragment thereof provided herein enhances the subject's LVEF by at least about 15%. In some embodiments, an anti-Sdc2 antibody or antigen-binding fragment thereof provided herein enhances the subject's LVEF by at least about 20%. In some embodiments, an anti-Sdc2 antibody or antigen-binding fragment thereof provided herein enhances the subject's LVEF by at least about 25%. In some embodiments, an anti-Sdc2 antibody or antigen-binding fragment thereof provided herein enhances the subject's LVEF by at least about 30%. In some embodiments, an anti-Sdc2 antibody or antigen-binding fragment thereof provided herein enhances the subject's LVEF by at least about 35%. In some embodiments, an anti-Sdc2 antibody or antigen-binding fragment thereof provided herein enhances the subject's LVEF by at least about 40%. In some embodiments, an anti-Sdc2 antibody or antigen-binding fragment thereof provided herein enhances the subject's LVEF by at least about 45%. In some embodiments, an anti-Sdc2 antibody or antigen-binding fragment thereof provided herein enhances the subject's LVEF by at least about 50%. In some embodiments, an anti-Sdc2 antibody or antigen-binding fragment thereof provided herein enhances the subject's LVEF by at least about 55%. In some embodiments, an anti-Sdc2 antibody or antigen-binding fragment thereof provided herein enhances the subject's LVEF by at least about 60%. In some embodiments, an anti-Sdc2 antibody or antigen-binding fragment thereof provided herein enhances the subject's LVEF by at least about 65%. In some embodiments, an anti-Sdc2 antibody or antigen-binding fragment thereof provided herein enhances the subject's LVEF by at least about 70%. In some embodiments, an anti-Sdc2 antibody or antigen-binding fragment thereof provided herein enhances the subject's LVEF by at least about 75%. In some embodiments, an anti-Sdc2 antibody or antigen-binding fragment thereof provided herein enhances the subject's LVEF by at least about 80%. In some embodiments, an anti-Sdc2 antibody or antigen-binding fragment thereof provided herein enhances the subject's LVEF by at least about 85%. In some embodiments, an anti-Sdc2 antibody or antigen-binding fragment thereof provided herein enhances the subject's LVEF by at least about 90%. In some embodiments, an anti-Sdc2 antibody or antigen-binding fragment thereof provided herein enhances the subject's LVEF by at least about 95%. In some embodiments, an anti-Sdc2 antibody or antigen-binding fragment thereof provided herein enhances the subject's LVEF by at least about 98%. In some embodiments, an anti-Sdc2 antibody or antigen-binding fragment thereof provided herein enhances the subject's LVEF by at least about 99%. In some embodiments, an anti-Sdc2 antibody or antigen-binding fragment thereof provided herein enhances the subject's LVEF by at least about 100%. In specific embodiments, the subject's LVEF is assessed by methods described herein. In some embodiments, the subject's LVEF is assessed by methods known to one of skill in the art. In some embodiments, the subject's LVEF is assessed using echocardiography following standards of the art. In some embodiments, LVEF is measured based on (LVEDV−LVESF)*HF.

In some embodiments, the antibody or antigen-binding fragment thereof, when bound to Sdc2, increases cardiac output of a subject suffered from AMI. In specific embodiments, antibodies provided herein (e.g., any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, or R4M-P1-A10 or an antigen-binding fragment thereof, or an antibody comprising CDRs of any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, and R4M-P1-A10) specifically bind to Sdc2 and enhance the subject's cardiac output.

In some embodiments, an anti-Sdc2 antibody or antigen-binding fragment thereof provided herein enhances the subject's cardiac output by at least about 10%. In some embodiments, an anti-Sdc2 antibody or antigen-binding fragment thereof provided herein enhances the subject's cardiac output by at least about 15%. In some embodiments, an anti-Sdc2 antibody or antigen-binding fragment thereof provided herein enhances the subject's cardiac output by at least about 20%. In some embodiments, an anti-Sdc2 antibody or antigen-binding fragment thereof provided herein enhances the subject's cardiac output by at least about 25%. In some embodiments, an anti-Sdc2 antibody or antigen-binding fragment thereof provided herein enhances the subject's cardiac output by at least about 30%. In some embodiments, an anti-Sdc2 antibody or antigen-binding fragment thereof provided herein enhances the subject's cardiac output by at least about 35%. In some embodiments, an anti-Sdc2 antibody or antigen-binding fragment thereof provided herein enhances the subject's cardiac output by at least about 40%. In some embodiments, an anti-Sdc2 antibody or antigen-binding fragment thereof provided herein enhances the subject's cardiac output by at least about 45%. In some embodiments, an anti-Sdc2 antibody or antigen-binding fragment thereof provided herein enhances the subject's cardiac output by at least about 50%. In some embodiments, an anti-Sdc2 antibody or antigen-binding fragment thereof provided herein enhances the subject's cardiac output by at least about 55%. In some embodiments, an anti-Sdc2 antibody or antigen-binding fragment thereof provided herein enhances the subject's cardiac output by at least about 60%. In some embodiments, an anti-Sdc2 antibody or antigen-binding fragment thereof provided herein enhances the subject's cardiac output by at least about 65%. In some embodiments, an anti-Sdc2 antibody or antigen-binding fragment thereof provided herein enhances the subject's cardiac output by at least about 70%. In some embodiments, an anti-Sdc2 antibody or antigen-binding fragment thereof provided herein enhances the subject's cardiac output by at least about 75%. In some embodiments, an anti-Sdc2 antibody or antigen-binding fragment thereof provided herein enhances the subject's cardiac output by at least about 80%. In some embodiments, an anti-Sdc2 antibody or antigen-binding fragment thereof provided herein enhances the subject's cardiac output by at least about 85%. In some embodiments, an anti-Sdc2 antibody or antigen-binding fragment thereof provided herein enhances the subject's cardiac output by at least about 90%. In some embodiments, an anti-Sdc2 antibody or antigen-binding fragment thereof provided herein enhances the subject's cardiac output by at least about 95%. In some embodiments, an anti-Sdc2 antibody or antigen-binding fragment thereof provided herein enhances the subject's cardiac output by at least about 98%. In some embodiments, an anti-Sdc2 antibody or antigen-binding fragment thereof provided herein enhances the subject's cardiac output by at least about 99%. In some embodiments, an anti-Sdc2 antibody or antigen-binding fragment thereof provided herein enhances the subject's cardiac output by at least about 100%. In specific embodiments, the subject's cardiac output is assessed by methods described herein. In some embodiments, the subject's cardiac output is assessed by methods known to one of skill in the art. In some embodiments, the subject's cardiac output is assessed using echocardiography following standards of the art.

In some embodiments, the antibody or antigen-binding fragment thereof, when bound to Sdc2, reduces the end diastolic LV internal diameter (LVIDd) or the LV end diastolic diameter (LVEDD) of a subject suffered from AMI. In specific embodiments, antibodies provided herein (e.g., any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, or R4M-P1-A10 or an antigen-binding fragment thereof, or an antibody comprising CDRs of any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, and R4M-P1-A10) specifically bind to Sdc2 and reduces the subject's LVIDd or LVEDD.

In some embodiments, an anti-Sdc2 antibody or antigen-binding fragment thereof provided herein reduces the subject's LVIDd or LVEDD by at least about 10%. In some embodiments, an anti-Sdc2 antibody or antigen-binding fragment thereof provided herein reduces the subject's LVIDd or LVEDD by at least about 15%. In some embodiments, an anti-Sdc2 antibody or antigen-binding fragment thereof provided herein reduces the subject's LVIDd or LVEDD by at least about 20%. In some embodiments, an anti-Sdc2 antibody or antigen-binding fragment thereof provided herein reduces the subject's LVIDd or LVEDD by at least about 25%. In some embodiments, an anti-Sdc2 antibody or antigen-binding fragment thereof provided herein reduces the subject's LVIDd or LVEDD by at least about 30%. In some embodiments, an anti-Sdc2 antibody or antigen-binding fragment thereof provided herein reduces the subject's LVIDd or LVEDD by at least about 35%. In some embodiments, an anti-Sdc2 antibody or antigen-binding fragment thereof provided herein reduces the subject's LVIDd or LVEDD by at least about 40%. In some embodiments, an anti-Sdc2 antibody or antigen-binding fragment thereof provided herein reduces the subject's LVIDd or LVEDD by at least about 45%. In some embodiments, an anti-Sdc2 antibody or antigen-binding fragment thereof provided herein reduces the subject's LVIDd or LVEDD by at least about 50%. In some embodiments, an anti-Sdc2 antibody or antigen-binding fragment thereof provided herein reduces the subject's LVIDd or LVEDD by at least about 55%. In some embodiments, an anti-Sdc2 antibody or antigen-binding fragment thereof provided herein reduces the subject's LVIDd or LVEDD by at least about 60%. In some embodiments, an anti-Sdc2 antibody or antigen-binding fragment thereof provided herein reduces the subject's LVIDd or LVEDD by at least about 65%. In some embodiments, an anti-Sdc2 antibody or antigen-binding fragment thereof provided herein reduces the subject's LVIDd or LVEDD by at least about 70%. In some embodiments, an anti-Sdc2 antibody or antigen-binding fragment thereof provided herein reduces the subject's LVIDd or LVEDD by at least about 75%. In some embodiments, an anti-Sdc2 antibody or antigen-binding fragment thereof provided herein reduces the subject's LVIDd or LVEDD by at least about 80%. In some embodiments, an anti-Sdc2 antibody or antigen-binding fragment thereof provided herein reduces the subject's LVIDd or LVEDD by at least about 85%. In some embodiments, an anti-Sdc2 antibody or antigen-binding fragment thereof provided herein reduces the subject's LVIDd or LVEDD at least about 90%. In some embodiments, an anti-Sdc2 antibody or antigen-binding fragment thereof provided herein reduces the subject's LVIDd or LVEDD by at least about 95%. In some embodiments, an anti-Sdc2 antibody or antigen-binding fragment thereof provided herein reduces the subject's LVIDd or LVEDD by at least about 98%. In some embodiments, an anti-Sdc2 antibody or antigen-binding fragment thereof provided herein reduces the subject's LVIDd or LVEDD by at least about 99%. In some embodiments, an anti-Sdc2 antibody or antigen-binding fragment thereof provided herein reduces the subject's LVIDd or LVEDD by at least about 100%. In specific embodiments, the subject's LVIDd or LVEDD is assessed by methods described herein. In some embodiments, the subject's LVIDd or LVEDD is assessed by methods known to one of skill in the art. In some embodiments, the subject's LVIDd or LVEDD is assessed using echocardiography following standards of the art. In some embodiments, the subject's LVIDd or LVEDD is measured as the distance of end diastolic diameter and volume in real time grey scale echo.

In some embodiments, the antibody or antigen-binding fragment thereof, when bound to Sdc2, reduces the end systolic LV internal diameter (LVIDs) or LV end systolic diameter (LVESD) of a subject suffered from AMI. In specific embodiments, antibodies provided herein (e.g., any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, or R4M-P1-A10 or an antigen-binding fragment thereof, or an antibody comprising CDRs of any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, and R4M-P1-A10) specifically bind to Sdc2 and reduces the subject's LVIDs or LVESD.

In some embodiments, an anti-Sdc2 antibody or antigen-binding fragment thereof provided herein reduces the subject's LVIDs or LVESD by at least about 10%. In some embodiments, an anti-Sdc2 antibody or antigen-binding fragment thereof provided herein reduces the subject's LVIDs or LVESD by at least about 15%. In some embodiments, an anti-Sdc2 antibody or antigen-binding fragment thereof provided herein reduces the subject's LVIDs or LVESD by at least about 20%. In some embodiments, an anti-Sdc2 antibody or antigen-binding fragment thereof provided herein reduces the subject's LVIDs or LVESD by at least about 25%. In some embodiments, an anti-Sdc2 antibody or antigen-binding fragment thereof provided herein reduces the subject's LVIDs or LVESD by at least about 30%. In some embodiments, an anti-Sdc2 antibody or antigen-binding fragment thereof provided herein reduces the subject's LVIDs or LVESD by at least about 35%. In some embodiments, an anti-Sdc2 antibody or antigen-binding fragment thereof provided herein reduces the subject's LVIDs or LVESD by at least about 40%. In some embodiments, an anti-Sdc2 antibody or antigen-binding fragment thereof provided herein reduces the subject's LVIDs or LVESD by at least about 45%. In some embodiments, an anti-Sdc2 antibody or antigen-binding fragment thereof provided herein reduces the subject's LVIDs or LVESD by at least about 50%. In some embodiments, an anti-Sdc2 antibody or antigen-binding fragment thereof provided herein reduces the subject's LVIDs or LVESD by at least about 55%. In some embodiments, an anti-Sdc2 antibody or antigen-binding fragment thereof provided herein reduces the subject's LVIDs or LVESD by at least about 60%. In some embodiments, an anti-Sdc2 antibody or antigen-binding fragment thereof provided herein reduces the subject's LVIDs or LVESD by at least about 65%. In some embodiments, an anti-Sdc2 antibody or antigen-binding fragment thereof provided herein reduces the subject's LVIDs or LVESD by at least about 70%. In some embodiments, an anti-Sdc2 antibody or antigen-binding fragment thereof provided herein reduces the subject's LVIDs or LVESD by at least about 75%. In some embodiments, an anti-Sdc2 antibody or antigen-binding fragment thereof provided herein reduces the subject's LVIDs or LVESD by at least about 80%. In some embodiments, an anti-Sdc2 antibody or antigen-binding fragment thereof provided herein reduces the subject's LVIDs or LVESD by at least about 85%. In some embodiments, an anti-Sdc2 antibody or antigen-binding fragment thereof provided herein reduces the subject's LVIDs or LVESD by at least about 90%. In some embodiments, an anti-Sdc2 antibody or antigen-binding fragment thereof provided herein reduces the subject's LVIDs or LVESD by at least about 95%. In some embodiments, an anti-Sdc2 antibody or antigen-binding fragment thereof provided herein reduces the subject's LVIDs or LVESD by at least about 98%. In some embodiments, an anti-Sdc2 antibody or antigen-binding fragment thereof provided herein reduces the subject's LVIDs or LVESD by at least about 99%. In some embodiments, an anti-Sdc2 antibody or antigen-binding fragment thereof provided herein reduces the subject's LVIDs or LVESD by at least about 100%. In specific embodiments, the subject's LVIDs or LVESD is assessed by methods described herein. In some embodiments, the subject's LVIDs or LVESD is assessed by methods known to one of skill in the art. In some embodiments, the subject's LVIDs or LVESD is assessed using echocardiography following standards of the art.

In some embodiments, the antibody or antigen-binding fragment thereof, when bound to Sdc2, increases the ejection fraction of the heart of a subject suffered from AMI. In specific embodiments, antibodies provided herein (e.g., any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, or R4M-P1-A10 or an antigen-binding fragment thereof, or an antibody comprising CDRs of any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, and R4M-P1-A10) specifically bind to Sdc2 and enhance the ejection fraction of the subject's heart.

In some embodiments, an anti-Sdc2 antibody or antigen-binding fragment thereof provided herein enhances the ejection fraction of the subject's heart by at least about 10%. In some embodiments, an anti-Sdc2 antibody or antigen-binding fragment thereof provided herein enhances the ejection fraction of the subject's heart by at least about 15%. In some embodiments, an anti-Sdc2 antibody or antigen-binding fragment thereof provided herein enhances the ejection fraction of the subject's heart by at least about 20%. In some embodiments, an anti-Sdc2 antibody or antigen-binding fragment thereof provided herein enhances the ejection fraction of the subject's heart by at least about 25%. In some embodiments, an anti-Sdc2 antibody or antigen-binding fragment thereof provided herein enhances the ejection fraction of the subject's heart by at least about 30%. In some embodiments, an anti-Sdc2 antibody or antigen-binding fragment thereof provided herein enhances the ejection fraction of the subject's heart by at least about 35%. In some embodiments, an anti-Sdc2 antibody or antigen-binding fragment thereof provided herein enhances the ejection fraction of the subject's heart by at least about 40%. In some embodiments, an anti-Sdc2 antibody or antigen-binding fragment thereof provided herein enhances the ejection fraction of the subject's heart by at least about 45%. In some embodiments, an anti-Sdc2 antibody or antigen-binding fragment thereof provided herein enhances the ejection fraction of the subject's heart by at least about 50%. In some embodiments, an anti-Sdc2 antibody or antigen-binding fragment thereof provided herein enhances the ejection fraction of the subject's heart by at least about 55%. In some embodiments, an anti-Sdc2 antibody or antigen-binding fragment thereof provided herein enhances the ejection fraction of the subject's heart by at least about 60%. In some embodiments, an anti-Sdc2 antibody or antigen-binding fragment thereof provided herein enhances the ejection fraction of the subject's heart by at least about 65%. In some embodiments, an anti-Sdc2 antibody or antigen-binding fragment thereof provided herein enhances the ejection fraction of the subject's heart by at least about 70%. In some embodiments, an anti-Sdc2 antibody or antigen-binding fragment thereof provided herein enhances the ejection fraction of the subject's heart by at least about 75%. In some embodiments, an anti-Sdc2 antibody or antigen-binding fragment thereof provided herein enhances the ejection fraction of the subject's heart by at least about 80%. In some embodiments, an anti-Sdc2 antibody or antigen-binding fragment thereof provided herein enhances the ejection fraction of the subject's heart by at least about 85%. In some embodiments, an anti-Sdc2 antibody or antigen-binding fragment thereof provided herein enhances the ejection fraction of the subject's heart by at least about 90%. In some embodiments, an anti-Sdc2 antibody or antigen-binding fragment thereof provided herein enhances the ejection fraction of the subject's heart by at least about 95%. In some embodiments, an anti-Sdc2 antibody or antigen-binding fragment thereof provided herein enhances the ejection fraction of the subject's heart by at least about 98%. In some embodiments, an anti-Sdc2 antibody or antigen-binding fragment thereof provided herein enhances the ejection fraction of the subject's heart by at least about 99%. In some embodiments, an anti-Sdc2 antibody or antigen-binding fragment thereof provided herein enhances the ejection fraction of the subject's heart by at least about 100%. In specific embodiments, the ejection fraction of the subject's heart is assessed by methods described herein. In some embodiments, the ejection fraction of the subject's heart is assessed by methods known to one of skill in the art. In some embodiments, the ejection fraction of the subject's heart is assessed using echocardiography following standards of the art. In some embodiments, the ejection fraction is measured as ((LVEDV−LVESV)/LVEDV)*100%.

In some embodiments, the antibody or antigen-binding fragment thereof, when bound to Sdc2, enhances the fractional shortening (FS) of the heart of a subject suffered from AMI. In specific embodiments, antibodies provided herein (e.g., any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, or R4M-P1-A10 or an antigen-binding fragment thereof, or an antibody comprising CDRs of any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, and R4M-P1-A10) specifically bind to Sdc2 and enhance the FS of the subject's heart.

In some embodiments, an anti-Sdc2 antibody or antigen-binding fragment thereof provided herein enhances the FS of the subject's heart by at least about 10%. In some embodiments, an anti-Sdc2 antibody or antigen-binding fragment thereof provided herein enhances the FS of the subject's heart by at least about 15%. In some embodiments, an anti-Sdc2 antibody or antigen-binding fragment thereof provided herein enhances the FS of the subject's heart by at least about 20%. In some embodiments, an anti-Sdc2 antibody or antigen-binding fragment thereof provided herein enhances the FS of the subject's heart by at least about 25%. In some embodiments, an anti-Sdc2 antibody or antigen-binding fragment thereof provided herein enhances the FS of the subject's heart by at least about 30%. In some embodiments, an anti-Sdc2 antibody or antigen-binding fragment thereof provided herein enhances the FS of the subject's heart by at least about 35%. In some embodiments, an anti-Sdc2 antibody or antigen-binding fragment thereof provided herein enhances the FS of the subject's heart by at least about 40%. In some embodiments, an anti-Sdc2 antibody or antigen-binding fragment thereof provided herein enhances the FS of the subject's heart by at least about 45%. In some embodiments, an anti-Sdc2 antibody or antigen-binding fragment thereof provided herein enhances the FS of the subject's heart by at least about 50%. In some embodiments, an anti-Sdc2 antibody or antigen-binding fragment thereof provided herein enhances the FS of the subject's heart by at least about 55%. In some embodiments, an anti-Sdc2 antibody or antigen-binding fragment thereof provided herein enhances the FS of the subject's heart by at least about 60%. In some embodiments, an anti-Sdc2 antibody or antigen-binding fragment thereof provided herein enhances the FS of the subject's heart by at least about 65%. In some embodiments, an anti-Sdc2 antibody or antigen-binding fragment thereof provided herein enhances the FS of the subject's heart by at least about 70%. In some embodiments, an anti-Sdc2 antibody or antigen-binding fragment thereof provided herein enhances the FS of the subject's heart by at least about 75%. In some embodiments, an anti-Sdc2 antibody or antigen-binding fragment thereof provided herein enhances the FS of the subject's heart by at least about 80%. In some embodiments, an anti-Sdc2 antibody or antigen-binding fragment thereof provided herein enhances the FS of the subject's heart by at least about 85%. In some embodiments, an anti-Sdc2 antibody or antigen-binding fragment thereof provided herein enhances the FS of the subject's heart by at least about 90%. In some embodiments, an anti-Sdc2 antibody or antigen-binding fragment thereof provided herein enhances the FS of the subject's heart by at least about 95%. In some embodiments, an anti-Sdc2 antibody or antigen-binding fragment thereof provided herein enhances the FS of the subject's heart by at least about 98%. In some embodiments, an anti-Sdc2 antibody or antigen-binding fragment thereof provided herein enhances the FS of the subject's heart by at least about 99%. In some embodiments, an anti-Sdc2 antibody or antigen-binding fragment thereof provided herein enhances the FS of the subject's heart by at least about 100%. In specific embodiments, the FS of the subject's heart is assessed by methods described herein. In some embodiments, the FS of the subject's heart is assessed by methods known to one of skill in the art. In some embodiments, the FS of the subject's heart is assessed using echocardiography following standards of the art. In some embodiments, the FS of the subject's heart is measured as ((LVEDD-LVESV)/LVEDD).

In some embodiments, the antibody or antigen-binding fragment thereof, when bound to Sdc2, reduces the LV mass of the heart of a subject suffered from AMI. In specific embodiments, antibodies provided herein (e.g., any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, or R4M-P1-A10 or an antigen-binding fragment thereof, or an antibody comprising CDRs of any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, and R4M-P1-A10) specifically bind to Sdc2 and reduce the LV mass of the subject's heart.

In some embodiments, an anti-Sdc2 antibody or antigen-binding fragment thereof provided herein reduces the LV mass of the subject's heart by at least about 10%. In some embodiments, an anti-Sdc2 antibody or antigen-binding fragment thereof provided herein reduces the LV mass of the subject's heart by at least about 15%. In some embodiments, an anti-Sdc2 antibody or antigen-binding fragment thereof provided herein reduces the LV mass of the subject's heart by at least about 20%. In some embodiments, an anti-Sdc2 antibody or antigen-binding fragment thereof provided herein reduces the LV mass of the subject's heart by at least about 25%. In some embodiments, an anti-Sdc2 antibody or antigen-binding fragment thereof provided herein reduces the LV mass of the subject's heart by at least about 30%. In some embodiments, an anti-Sdc2 antibody or antigen-binding fragment thereof provided herein reduces the LV mass of the subject's heart by at least about 35%. In some embodiments, an anti-Sdc2 antibody or antigen-binding fragment thereof provided herein reduces the LV mass of the subject's heart by at least about 40%. In some embodiments, an anti-Sdc2 antibody or antigen-binding fragment thereof provided herein reduces the LV mass of the subject's heart by at least about 45%. In some embodiments, an anti-Sdc2 antibody or antigen-binding fragment thereof provided herein reduces the LV mass of the subject's heart by at least about 50%. In some embodiments, an anti-Sdc2 antibody or antigen-binding fragment thereof provided herein reduces the LV mass of the subject's heart by at least about 55%. In some embodiments, an anti-Sdc2 antibody or antigen-binding fragment thereof provided herein reduces the LV mass of the subject's heart by at least about 60%. In some embodiments, an anti-Sdc2 antibody or antigen-binding fragment thereof provided herein reduces the LV mass of the subject's heart by at least about 65%. In some embodiments, an anti-Sdc2 antibody or antigen-binding fragment thereof provided herein reduces the LV mass of the subject's heart by at least about 70%. In some embodiments, an anti-Sdc2 antibody or antigen-binding fragment thereof provided herein reduces the LV mass of the subject's heart by at least about 75%. In some embodiments, an anti-Sdc2 antibody or antigen-binding fragment thereof provided herein reduces the LV mass of the subject's heart by at least about 80%. In some embodiments, an anti-Sdc2 antibody or antigen-binding fragment thereof provided herein reduces the LV mass of the subject's heart by at least about 85%. In some embodiments, an anti-Sdc2 antibody or antigen-binding fragment thereof provided herein reduces the LV mass of the subject's heart by at least about 90%. In some embodiments, an anti-Sdc2 antibody or antigen-binding fragment thereof provided herein reduces the LV mass of the subject's heart by at least about 95%. In some embodiments, an anti-Sdc2 antibody or antigen-binding fragment thereof provided herein reduces the LV mass of the subject's heart by at least about 98%. In some embodiments, an anti-Sdc2 antibody or antigen-binding fragment thereof provided herein reduces the LV mass of the subject's heart by at least about 99%. In some embodiments, an anti-Sdc2 antibody or antigen-binding fragment thereof provided herein reduces the LV mass of the subject's heart by at least about 100%. In specific embodiments, the LV mass of the subject's heart is assessed by methods described herein. In some embodiments, the LV mass of the subject's heart is assessed by methods known to one of skill in the art. In some embodiments, the LV mass of the subject's heart is assessed using echocardiography following standards of the art. In some embodiments, the LV mass is measured via volumetric grey scale analysis.

In some embodiments, the antibody or antigen-binding fragment thereof, when bound to Sdc2, reduces the LV end diastolic volume of the heart of a subject suffered from AMI. In specific embodiments, antibodies provided herein (e.g., any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, or R4M-P1-A10 or an antigen-binding fragment thereof, or an antibody comprising CDRs of any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, and R4M-P1-A10) specifically bind to Sdc2 and reduce the LV end diastolic volume of the subject's heart.

In some embodiments, an anti-Sdc2 antibody or antigen-binding fragment thereof provided herein reduces the LV end diastolic volume of the subject's heart by at least about 10%. In some embodiments, an anti-Sdc2 antibody or antigen-binding fragment thereof provided herein reduces the LV end diastolic volume of the subject's heart by at least about 15%. In some embodiments, an anti-Sdc2 antibody or antigen-binding fragment thereof provided herein reduces the LV end diastolic volume of the subject's heart by at least about 20%. In some embodiments, an anti-Sdc2 antibody or antigen-binding fragment thereof provided herein reduces the LV end diastolic volume of the subject's heart by at least about 25%. In some embodiments, an anti-Sdc2 antibody or antigen-binding fragment thereof provided herein reduces the LV end diastolic volume of the subject's heart by at least about 30%. In some embodiments, an anti-Sdc2 antibody or antigen-binding fragment thereof provided herein reduces the LV end diastolic volume of the subject's heart by at least about 35%. In some embodiments, an anti-Sdc2 antibody or antigen-binding fragment thereof provided herein reduces the LV end diastolic volume of the subject's heart by at least about 40%. In some embodiments, an anti-Sdc2 antibody or antigen-binding fragment thereof provided herein reduces the LV end diastolic volume of the subject's heart by at least about 45%. In some embodiments, an anti-Sdc2 antibody or antigen-binding fragment thereof provided herein reduces the LV end diastolic volume of the subject's heart by at least about 50%. In some embodiments, an anti-Sdc2 antibody or antigen-binding fragment thereof provided herein reduces the LV end diastolic volume of the subject's heart by at least about 55%. In some embodiments, an anti-Sdc2 antibody or antigen-binding fragment thereof provided herein reduces the LV end diastolic volume of the subject's heart by at least about 60%. In some embodiments, an anti-Sdc2 antibody or antigen-binding fragment thereof provided herein reduces the LV end diastolic volume of the subject's heart by at least about 65%. In some embodiments, an anti-Sdc2 antibody or antigen-binding fragment thereof provided herein reduces the LV end diastolic volume of the subject's heart by at least about 70%. In some embodiments, an anti-Sdc2 antibody or antigen-binding fragment thereof provided herein reduces the LV end diastolic volume of the subject's heart by at least about 75%. In some embodiments, an anti-Sdc2 antibody or antigen-binding fragment thereof provided herein reduces the LV end diastolic volume of the subject's heart by at least about 80%. In some embodiments, an anti-Sdc2 antibody or antigen-binding fragment thereof provided herein reduces the LV end diastolic volume of the subject's heart by at least about 85%. In some embodiments, an anti-Sdc2 antibody or antigen-binding fragment thereof provided herein reduces the LV end diastolic volume of the subject's heart by at least about 90%. In some embodiments, an anti-Sdc2 antibody or antigen-binding fragment thereof provided herein reduces the LV end diastolic volume of the subject's heart by at least about 95%. In some embodiments, an anti-Sdc2 antibody or antigen-binding fragment thereof provided herein reduces the LV end diastolic volume of the subject's heart by at least about 98%. In some embodiments, an anti-Sdc2 antibody or antigen-binding fragment thereof provided herein reduces the LV end diastolic volume of the subject's heart by at least about 99%. In some embodiments, an anti-Sdc2 antibody or antigen-binding fragment thereof provided herein reduces the LV end diastolic volume of the subject's heart by at least about 100%. In specific embodiments, the LV end diastolic volume of the subject's heart is assessed by methods described herein. In some embodiments, the LV end diastolic volume of the subject's heart is assessed by methods known to one of skill in the art. In some embodiments, the LV end diastolic volume of the subject's heart is assessed using echocardiography following standards of the art. In some embodiments, the LV end diastolic volume of the subject's heart is assessed by tracing or auto contour detection in conventional grey scale echocardiography in end diastole.

In some embodiments, the antibody or antigen-binding fragment thereof, when bound to Sdc2, reduces the LV end systolic volume of the heart of a subject suffered from AMI. In specific embodiments, antibodies provided herein (e.g., any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, or R4M-P1-A10 or an antigen-binding fragment thereof, or an antibody comprising CDRs of any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, and R4M-P1-A10) specifically bind to Sdc2 and reduce the LV end systolic volume of the subject's heart.

In some embodiments, an anti-Sdc2 antibody or antigen-binding fragment thereof provided herein reduces the LV end systolic volume of the subject's heart by at least about 10%. In some embodiments, an anti-Sdc2 antibody or antigen-binding fragment thereof provided herein reduces the LV end systolic volume of the subject's heart by at least about 15%. In some embodiments, an anti-Sdc2 antibody or antigen-binding fragment thereof provided herein reduces the LV end systolic volume of the subject's heart by at least about 20%. In some embodiments, an anti-Sdc2 antibody or antigen-binding fragment thereof provided herein reduces the LV end systolic volume of the subject's heart by at least about 25%. In some embodiments, an anti-Sdc2 antibody or antigen-binding fragment thereof provided herein reduces the LV end systolic volume of the subject's heart by at least about 30%. In some embodiments, an anti-Sdc2 antibody or antigen-binding fragment thereof provided herein reduces the LV end systolic volume of the subject's heart by at least about 35%. In some embodiments, an anti-Sdc2 antibody or antigen-binding fragment thereof provided herein reduces the LV end systolic volume of the subject's heart by at least about 40%. In some embodiments, an anti-Sdc2 antibody or antigen-binding fragment thereof provided herein reduces the LV end systolic volume of the subject's heart by at least about 45%. In some embodiments, an anti-Sdc2 antibody or antigen-binding fragment thereof provided herein reduces the LV end systolic volume of the subject's heart by at least about 50%. In some embodiments, an anti-Sdc2 antibody or antigen-binding fragment thereof provided herein reduces the LV end systolic volume of the subject's heart by at least about 55%. In some embodiments, an anti-Sdc2 antibody or antigen-binding fragment thereof provided herein reduces the LV end systolic volume of the subject's heart by at least about 60%. In some embodiments, an anti-Sdc2 antibody or antigen-binding fragment thereof provided herein reduces the LV end systolic volume of the subject's heart by at least about 65%. In some embodiments, an anti-Sdc2 antibody or antigen-binding fragment thereof provided herein reduces the LV end systolic volume of the subject's heart by at least about 70%. In some embodiments, an anti-Sdc2 antibody or antigen-binding fragment thereof provided herein reduces the LV end systolic volume of the subject's heart by at least about 75%. In some embodiments, an anti-Sdc2 antibody or antigen-binding fragment thereof provided herein reduces the LV end systolic volume of the subject's heart by at least about 80%. In some embodiments, an anti-Sdc2 antibody or antigen-binding fragment thereof provided herein reduces the LV end systolic volume of the subject's heart by at least about 85%. In some embodiments, an anti-Sdc2 antibody or antigen-binding fragment thereof provided herein reduces the LV end systolic volume of the subject's heart by at least about 90%. In some embodiments, an anti-Sdc2 antibody or antigen-binding fragment thereof provided herein reduces the LV end systolic volume of the subject's heart by at least about 95%. In some embodiments, an anti-Sdc2 antibody or antigen-binding fragment thereof provided herein reduces the LV end systolic volume of the subject's heart by at least about 98%. In some embodiments, an anti-Sdc2 antibody or antigen-binding fragment thereof provided herein reduces the LV end systolic volume of the subject's heart by at least about 99%. In some embodiments, an anti-Sdc2 antibody or antigen-binding fragment thereof provided herein reduces the LV end systolic volume of the subject's heart by at least about 100%. In specific embodiments, the LV end systolic volume of the subject's heart is assessed by methods described herein. In some embodiments, the LV end systolic volume of the subject's heart is assessed by methods known to one of skill in the art. In some embodiments, the LV end systolic volume of the subject's heart is assessed using echocardiography following standards of the art. In some embodiments, the LV end systolic volume of the subject's heart is assessed by tracing or auto contour detection in conventional grey scale echocardiography in end systole.

In some embodiments, the antibody or antigen-binding fragment thereof, when bound to Sdc2, reduces the risk of post-infarction ventricular tachycardia (VT) in a subject suffered from AMI. In specific embodiments, antibodies provided herein (e.g., any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, or R4M-P1-A10 or an antigen-binding fragment thereof, or an antibody comprising CDRs of any one of antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, and R4M-P1-A10) specifically bind to Sdc2 and reduce the risk of post-infarction VT in the subject.

In some embodiments, an anti-Sdc2 antibody or antigen-binding fragment thereof provided herein reduces the risk of post-infarction VT by at least about 10%. In some embodiments, an anti-Sdc2 antibody or antigen-binding fragment thereof provided herein reduces the risk of post-infarction VT by at least about 15%. In some embodiments, an anti-Sdc2 antibody or antigen-binding fragment thereof provided herein reduces the risk of post-infarction VT by at least about 20%. In some embodiments, an anti-Sdc2 antibody or antigen-binding fragment thereof provided herein reduces the risk of post-infarction VT by at least about 25%. In some embodiments, an anti-Sdc2 antibody or antigen-binding fragment thereof provided herein reduces the risk of post-infarction VT by at least about 30%. In some embodiments, an anti-Sdc2 antibody or antigen-binding fragment thereof provided herein reduces the risk of post-infarction VT by at least about 35%. In some embodiments, an anti-Sdc2 antibody or antigen-binding fragment thereof provided herein reduces the risk of post-infarction VT by at least about 40%. In some embodiments, an anti-Sdc2 antibody or antigen-binding fragment thereof provided herein reduces the risk of post-infarct VT by at least about 45%. In some embodiments, an anti-Sdc2 antibody or antigen-binding fragment thereof provided herein reduces the risk of post-infarction VT by at least about 50%. In some embodiments, an anti-Sdc2 antibody or antigen-binding fragment thereof provided herein reduces the risk of post-infarction VT by at least about 55%. In some embodiments, an anti-Sdc2 antibody or antigen-binding fragment thereof provided herein reduces the risk of post-infarction VT by at least about 60%. In some embodiments, an anti-Sdc2 antibody or antigen-binding fragment thereof provided herein reduces the risk of post-infarction VT by at least about 65%. In some embodiments, an anti-Sdc2 antibody or antigen-binding fragment thereof provided herein reduces the risk of post-infarction VT by at least about 70%. In some embodiments, an anti-Sdc2 antibody or antigen-binding fragment thereof provided herein reduces the risk of post-infarction VT by at least about 75%. In some embodiments, an anti-Sdc2 antibody or antigen-binding fragment thereof provided herein reduces the risk of post-infarction VT by at least about 80%. In some embodiments, an anti-Sdc2 antibody or antigen-binding fragment thereof provided herein reduces the risk of post-infarction VT by at least about 85%. In some embodiments, an anti-Sdc2 antibody or antigen-binding fragment thereof provided herein reduces the risk of post-infarction VT by at least about 90%. In some embodiments, an anti-Sdc2 antibody or antigen-binding fragment thereof provided herein reduces the risk of post-infarction VT by at least about 95%. In some embodiments, an anti-Sdc2 antibody or antigen-binding fragment thereof provided herein reduces the risk of post-infarction VT by at least about 98%. In some embodiments, an anti-Sdc2 antibody or antigen-binding fragment thereof provided herein reduces the risk of post-infarction VT by at least about 99%. In some embodiments, an anti-Sdc2 antibody or antigen-binding fragment thereof provided herein reduces the risk of post-infarction VT by at least about 100%. In specific embodiments, the risk of post-infarction VT is assessed by methods described herein. In some embodiments, the risk of post-infarction VT is assessed by methods known to one of skill in the art. In some embodiment, the risk of post-infarction VT is assessed using electrocardiogram with programed stimulation. In some embodiments, an enhanced threshold of voltage needed for inducing VT in the subject indicates a reduced risk of post-infarction VT in the subject. In some embodiments, an enhanced number of stimuli needed to induce VT in the subject indicates a reduced risk of post-infarction VT in the subject. In some embodiments, a reduced duration for VT induced by programed stimulation in the subject indicates a reduced risk of post-infarction VT in the subject.

### 6.3.1 Polyclonal Antibodies

The antibodies of the present disclosure may comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include a Sdc2 polypeptide or a fusion protein thereof. It may be useful to conjugate the immunizing agent to a protein known to be immunogenic in the mammal being immunized or to immunize the mammal with the protein and one or more adjuvants. Examples of such immunogenic proteins include, but are not limited to, keyhole limpet hemocyanin, serum albumin, bovine thyroglobulin, and soybean trypsin inhibitor. Examples of adjuvants which may be employed include Ribi, CpG, Poly 1C, Freund's complete adjuvant, and MPL-TDM adjuvant (monophosphoryl Lipid A, synthetic trehalose dicorynomycolate). The immunization protocol may be selected by one skilled in the art without undue experimentation. The mammal can then be bled, and the serum assayed for Sdc2 antibody titer. If desired, the mammal can be boosted until the antibody titer increases or plateaus. Additionally or alternatively, lymphocytes may be obtained from the immunized animal for fusion and preparation of monoclonal antibodies from hybridoma as described below.

### 6.3.2 Monoclonal Antibodies

The antibodies of the present disclosure may alternatively be monoclonal antibodies. Monoclonal antibodies may be made using the hybridoma method first described by Kohler et al., 1975, Nature 256:495-97, or may be made by recombinant DNA methods (see, e.g., U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice 59-103 (1986)).

The hybridoma cells thus prepared are seeded and grown in a suitable culture medium which, in certain embodiments, contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which prevent the growth of HGPRT-deficient cells.

Exemplary fusion partner myeloma cells are those that fuse efficiently, support stable high-level production of antibody by the selected antibody-producing cells, and are sensitive to a selective medium that selects against the unfused parental cells. Exemplary myeloma cell lines are murine myeloma lines, such as SP-2 and derivatives, for example, X63-Ag8-653 cells available from the American Type Culture Collection (Manassas, VA), and those derived from MOPC-21 and MPC-11 mouse tumors available from the Salk Institute Cell Distribution Center (San Diego, CA). Human myeloma and mouse-human heteromyeloma cell lines also have been described for the production of human monoclonal antibodies (Kozbor, 1984, Immunol. 133:3001-05; and Brodeur et al., Monoclonal Antibody Production Techniques and Applications 51-63 (1987)).

Culture medium in which hybridoma cells are growing is assayed for production of monoclonal antibodies directed against the antigen. The binding specificity of monoclonal antibodies produced by hybridoma cells is determined by immunoprecipitation or by an in vitro binding assay, such as RIA or ELISA. The binding affinity of the monoclonal antibody can, for example, be determined by the Scatchard analysis described in Munson et al., 1980, Anal. Biochem. 107:220-39.

Once hybridoma cells that produce antibodies of the desired specificity, affinity, and/or activity are identified, the clones may be subcloned by limiting dilution procedures and grown by standard methods (Goding, supra). Suitable culture media for this purpose include, for example, DMEM or RPMI-1640 medium. In addition, the hybridoma cells may be grown in vivo as ascites tumors in an animal, for example, by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells can serve as a source of such DNA. Once isolated, the DNA may be placed into expression vectors, which are then transfected into host cells, such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., 1993, Curr. Opinion in Immunol. 5:256-62 and Pluckthun, 1992, Immunol. Revs. 130:151-88.

In some embodiments, an antibody that binds a Sdc2 epitope comprises an amino acid sequence of a VH domain and/or an amino acid sequence of a VL domain encoded by a nucleotide sequence that hybridizes to (1) the complement of a nucleotide sequence encoding any one of the VH and/or VL domain described herein under stringent conditions (e.g., hybridization to filter-bound DNA in 6× sodium chloride/sodium citrate (SSC) at about 45° C. followed by one or more washes in 0.2×SSC/0.1% SDS at about 50-65° C.), under highly stringent conditions (e.g., hybridization to filter-bound nucleic acid in 6×SSC at about 45° C. followed by one or more washes in 0.1×SSC/0.2% SDS at about 68° C.), or under other stringent hybridization conditions which are known to those of skill in the art. See, e.g., Current Protocols in Molecular Biology Vol. I, 6.3.1-6.3.6 and 2.10.3 (Ausubel et al. eds., 1989).

In some embodiments, an antibody that binds a Sdc2 epitope comprises an amino acid sequence of a VH CDR or an amino acid sequence of a VL CDR encoded by a nucleotide sequence that hybridizes to the complement of a nucleotide sequence encoding any one of the VH CDRs and/or VL CDRs depicted in Tables 4-8 under stringent conditions (e.g., hybridization to filter-bound DNA in 6×SSC at about 45° C. followed by one or more washes in 0.2×SSC/0.1% SDS at about 50-65° C.), under highly stringent conditions (e.g., hybridization to filter-bound nucleic acid in 6×SSC at about 45° C. followed by one or more washes in 0.1×SSC/0.2% SDS at about 68° C.), or under other stringent hybridization conditions which are known to those of skill in the art (see, e.g., Ausubel et al., supra).

In a further embodiment, monoclonal antibodies or antibody fragments can be isolated from antibody phage libraries generated using the techniques described in, for example, Antibody Phage Display: Methods and Protocols (O'Brien and Aitken eds., 2002). In principle, synthetic antibody clones are selected by screening phage libraries containing phages that display various fragments of antibody variable region (Fv) fused to phage coat protein. Such phage libraries are screened against the desired antigen. Clones expressing Fv fragments capable of binding to the desired antigen are adsorbed to the antigen and thus separated from the non-binding clones in the library. The binding clones are then eluted from the antigen and can be further enriched by additional cycles of antigen adsorption/elution.

Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described, for example, in Winter et al., 1994, Ann. Rev. Immunol. 12:433-55.

Repertoires of VH and VL genes can be separately cloned by PCR and recombined randomly in phage libraries, which can then be searched for antigen-binding clones as described in Winter et al., supra. Libraries from immunized sources provide high-affinity antibodies to the immunogen without the requirement of constructing hybridomas. Alternatively, the naive repertoire can be cloned to provide a single source of human antibodies to a wide range of non-self and also self-antigens without any immunization as described by Griffiths et al., 1993, EMBO J 12:725-34. Finally, naive libraries can also be made synthetically by cloning the unrearranged V-gene segments from stem cells and using PCR primers containing random sequence to encode the highly variable CDR3 regions and to accomplish rearrangement in vitro as described, for example, by Hoogenboom and Winter, 1992, J. Mol. Biol. 227:381-88.

Screening of the libraries can be accomplished by various techniques known in the art. For example, Sdc2 (e.g., a Sdc2 polypeptide, fragment, or epitope) can be used to coat the wells of adsorption plates, expressed on host cells affixed to adsorption plates or used in cell sorting, conjugated to biotin for capture with streptavidin-coated beads, or used in any other method for panning display libraries. The selection of antibodies with slow dissociation kinetics (e.g., good binding affinities) can be promoted by use of long washes and monovalent phage display as described in Bass et al., 1990, Proteins 8:309-14 and WO 92/09690, and by use of a low coating density of antigen as described in Marks et al., 1992, Biotechnol. 10:779-83.

Anti-Sdc2 antibodies can be obtained by designing a suitable antigen screening procedure to select for the phage clone of interest followed by construction of a full length anti-Sdc2 antibody clone using VH and/or VL sequences (e.g., the Fv sequences), or various CDR sequences from VH and VL sequences, from the phage clone of interest and suitable constant region (e.g., Fc) sequences described in Kabat et al., supra.

In another embodiment, anti-Sdc2 antibody is generated by using methods as described in Bowers et al., 2011, Proc Natl Acad Sci USA. 108:20455-60, e.g., the SHM-XHL™ platform (AnaptysBio, San Diego, CA). Briefly, in this approach, a fully human library of IgGs is constructed in a mammalian cell line (e.g., HEK293) as a starting library. Mammalian cells displaying immunoglobulin that binds to a target peptide or epitope are selected (e.g., by FACS sorting), then activation-induced cytidine deaminase (AID)-triggered somatic hypermutation is reproduced in vitro to expand diversity of the initially selected pool of antibodies. After several rounds of affinity maturation by coupling mammalian cell surface display with in vitro somatic hypermutation, high affinity, high specificity anti-Sdc2 antibodies are generated. Further methods that can be used to generate antibody libraries and/or antibody affinity maturation are disclosed, e.g., in U.S. Pat. Nos. 8,685,897 and 8,603,930, and U.S. Publ. Nos. 2014/0170705, 2014/0094392, 2012/0028301, 2011/0183855, and 2009/0075378, each of which are incorporated herein by reference.

### 6.3.3 Antibody Fragments

The present disclosure provides antibodies and antibody fragments that bind to Sdc2. In certain circumstances there are advantages of using antibody fragments, rather than whole antibodies. The smaller size of the fragments allows for rapid clearance, and may lead to improved access to cells, tissues, or organs. For a review of certain antibody fragments, see Hudson et al., 2003, Nature Med. 9:129-34.

Various techniques have been developed for the production of antibody fragments. Traditionally, these fragments were derived via proteolytic digestion of intact antibodies (see, e.g., Morimoto et al., 1992, J. Biochem. Biophys. Methods 24:107-17; and Brennan et al., 1985, Science 229:81-83). However, these fragments can now be produced directly by recombinant host cells. Fab, Fv, and scFv antibody fragments can all be expressed in and secreted from E. coli or yeast cells, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., 1992, Bio/Technology 10:163-67). According to another approach, F(ab′)₂ fragments can be isolated directly from recombinant host cell culture. Fab and F(ab′)₂ fragment with increased in vivo half-life comprising salvage receptor binding epitope residues are described in, for example, U.S. Pat. No. 5,869,046. Other techniques for the production of antibody fragments will be apparent to the skilled practitioner. In certain embodiments, an antibody is a single chain Fv fragment (scFv) (see, e.g., WO 93/16185; U.S. Pat. Nos. 5,571,894 and 5,587,458). Fv and scFv have intact combining sites that are devoid of constant regions; thus, they may be suitable for reduced nonspecific binding during in vivo use. scFv fusion proteins may be constructed to yield fusion of an effector protein at either the amino or the carboxy terminus of an scFv (See, e.g., Borrebaeck ed., supra). The antibody fragment may also be a “linear antibody,” for example, as described in the references cited above. Such linear antibodies may be monospecific or multi-specific, such as bispecific.

Smaller antibody-derived binding structures are the separate variable domains (V domains) also termed single variable domain antibodies (sdAbs). Certain types of organisms, the camelids and cartilaginous fish, possess high affinity single V-like domains mounted on an Fc equivalent domain structure as part of their immune system. (Woolven et al., 1999, Immunogenetics 50:98-101; and Streltsov et al., 2004, Proc Natl Acad Sci USA. 101:12444-49). The V-like domains (called VhH in camelids and V-NAR in sharks) typically display long surface loops, which allow penetration of cavities of target antigens. They also stabilize isolated VH domains by masking hydrophobic surface patches.

These VhH and V-NAR domains have been used to engineer sdAbs. Human V domain variants have been designed using selection from phage libraries and other approaches that have resulted in stable, high binding VL- and VH-derived domains.

Antibodies provided herein include, but are not limited to, immunoglobulin molecules and immunologically active portions of immunoglobulin molecules, for example, molecules that contain an antigen binding site that bind to a Sdc2 epitope. The immunoglobulin molecules provided herein can be of any class (e.g., IgG, IgE, IgM, IgD, and IgA) or any subclass (e.g., IgG1, IgG2, IgG3, IgG4, IgA1, and IgA2) of immunoglobulin molecule.

Variants and derivatives of antibodies include antibody functional fragments that retain the ability to bind to a Sdc2 epitope. Exemplary functional fragments include Fab fragments (e.g., an antibody fragment that contains the antigen-binding domain and comprises a light chain and part of a heavy chain bridged by a disulfide bond); Fab′ (e.g., an antibody fragment containing a single antigen-binding domain comprising an Fab and an additional portion of the heavy chain through the hinge region); F(ab′)₂ (e.g., two Fab′ molecules joined by interchain disulfide bonds in the hinge regions of the heavy chains; the Fab′ molecules may be directed toward the same or different epitopes); a bispecific Fab (e.g., a Fab molecule having two antigen binding domains, each of which may be directed to a different epitope); a single chain comprising a variable region, also known as, scFv (e.g., the variable, antigen-binding determinative region of a single light and heavy chain of an antibody linked together by a chain of 10-25 amino acids); a disulfide-linked Fv, or dsFv (e.g., the variable, antigen-binding determinative region of a single light and heavy chain of an antibody linked together by a disulfide bond); a camelized VH (e.g., the variable, antigen-binding determinative region of a single heavy chain of an antibody in which some amino acids at the VH interface are those found in the heavy chain of naturally occurring camel antibodies); a bispecific scFv (e.g., an scFv or a dsFv molecule having two antigen-binding domains, each of which may be directed to a different epitope); a diabody (e.g., a dimerized scFv formed when the VH domain of a first scFv assembles with the VL domain of a second scFv and the VL domain of the first scFv assembles with the VH domain of the second scFv; the two antigen-binding regions of the diabody may be directed towards the same or different epitopes); and a triabody (e.g., a trimerized scFv, formed in a manner similar to a diabody, but in which three antigen-binding domains are created in a single complex; the three antigen-binding domains may be directed towards the same or different epitopes).

### 6.3.4 Humanized Antibodies

In some embodiments, antibodies provided herein can be humanized antibodies that bind Sdc2, including human and/or cyno Sdc2. For example, humanized antibodies of the present disclosure may comprise one or more CDRs as shown in Tables 4-8. Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanization may be performed, for example, following the method of Jones et al., 1986, Nature 321:522-25; Riechmann et al., 1988, Nature 332:323-27; and Verhoeyen et al., 1988, Science 239:1534-36), by substituting hypervariable region sequences for the corresponding sequences of a human antibody.

In some cases, the humanized antibodies are constructed by CDR grafting, in which the amino acid sequences of the six CDRs of the parent non-human antibody (e.g., rodent) are grafted onto a human antibody framework. For example, Padlan et al. determined that only about one third of the residues in the CDRs actually contact the antigen, and termed these the “specificity determining residues,” or SDRs (Padlan et al., 1995, FASEB J. 9:133-39). In the technique of SDR grafting, only the SDR residues are grafted onto the human antibody framework (see, e.g., Kashmiri et al., 2005, Methods 36:25-34).

The choice of human variable domains, both light and heavy, to be used in making the humanized antibodies can be important to reduce antigenicity. For example, according to the so-called “best-fit” method, the sequence of the variable domain of a non-human (e.g., rodent) antibody is screened against the entire library of known human variable-domain sequences. The human sequence that is closest to that of the rodent may be selected as the human framework for the humanized antibody (Sims et al., 1993, J. Immunol. 151:2296-308; and Chothia et al., 1987, J. Mol. Biol. 196:901-17). Another method uses a particular framework derived from the consensus sequence of all human antibodies of a particular subgroup of light or heavy chains. The same framework may be used for several different humanized antibodies (Carter et al., 1992, Proc. Natl. Acad. Sci. USA 89:4285-89; and Presta et al., 1993, J. Immunol. 151:2623-32). In some cases, the framework is derived from the consensus sequences of the most abundant human subclasses, V_{L}6 subgroup I (V_{L}6I) and V_{H} subgroup III (V_{H}III). In another method, human germline genes are used as the source of the framework regions.

In an alternative paradigm based on comparison of CDRs, called superhumanization, FR homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs (see, e.g., Tan et al., 2002, J. Immunol. 169:1119-25).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, 2000, Protein Eng. 13:819-24), Modeller (Sali and Blundell, 1993, J. Mol. Biol. 234:779-815), and Swiss PDB Viewer (Guex and Peitsch, 1997, Electrophoresis 18:2714-23). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes, and the differences are scored as HSC. The target sequence is then humanized by maximizing its HSC rather than using a global identity measure to generate multiple diverse humanized variants (Lazar et al., 2007, Mol. Immunol. 44:1986-98).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome, and yeast display libraries as well as by bacterial colony screening (see, e.g., Hoogenboom, 2005, Nat. Biotechnol. 23:1105-16; Dufner et al., 2006, Trends Biotechnol. 24:523-29; Feldhaus et al., 2003, Nat. Biotechnol. 21:163-70; and Schlapschy et al., 2004, Protein Eng. Des. Sel. 17:847-60).

In the FR library approach, a collection of residue variants is introduced at specific positions in the FR followed by screening of the library to select the FR that best supports the grafted CDR. The residues to be substituted may include some or all of the “Vernier” residues identified as potentially contributing to CDR structure (see, e.g., Foote and Winter, 1992, J. Mol. Biol. 224:487-99), or from the more limited set of target residues identified by Baca et al. (1997, J. Biol. Chem. 272:10678-84).

In FR shuffling, whole FRs are combined with the non-human CDRs instead of creating combinatorial libraries of selected residue variants (see, e.g., Dall'Acqua et al., 2005, Methods 36:43-60). The libraries may be screened for binding in a two-step process, first humanizing VL, followed by VH. Alternatively, a one-step FR shuffling process may be used. Such a process has been shown to be more efficient than the two-step screening, as the resulting antibodies exhibited improved biochemical and physicochemical properties including enhanced expression, increased affinity, and thermal stability (see, e.g., Damschroder et al., 2007, Mol. Immunol. 44:3049-60).

The “humaneering” method is based on experimental identification of essential minimum specificity determinants (MSDs) and is based on sequential replacement of non-human fragments into libraries of human FRs and assessment of binding. It begins with regions of the CDR3 of non-human VH and VL chains and progressively replaces other regions of the non-human antibody into the human FRs, including the CDR1 and CDR2 of both VH and VL. This methodology typically results in epitope retention and identification of antibodies from multiple subclasses with distinct human V-segment CDRs. Humaneering allows for isolation of antibodies that are 91-96% homologous to human germline gene antibodies (see, e.g., Alfenito, Cambridge Healthtech Institute's Third Annual PEGS, The Protein Engineering Summit, 2007).

The “human engineering” method involves altering a non-human antibody or antibody fragment, such as a mouse or chimeric antibody or antibody fragment, by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human (e.g., mouse) antibody as “low risk,” “moderate risk,” or “high risk” residues. The classification is performed using a global risk/reward calculation that evaluates the predicted benefits of making particular substitution (e.g., for immunogenicity in humans) against the risk that the substitution will affect the resulting antibody's folding. The particular human amino acid residue to be substituted at a given position (e.g., low or moderate risk) of a non-human (e.g., mouse) antibody sequence can be selected by aligning an amino acid sequence from the non-human antibody's variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., 1994, Protein Engineering 7:805-14; U.S. Pat. Nos. 5,766,886; 5,770,196; 5,821,123; and 5,869,619; and PCT Publication WO 93/11794.

### 6.3.5 Human Antibodies

Human anti-Sdc2 antibodies can be constructed by combining Fv clone variable domain sequence(s) selected from human-derived phage display libraries with known human constant domain sequences(s). Alternatively, human monoclonal anti-Sdc2 antibodies of the present disclosure can be made by the hybridoma method. Human myeloma and mouse-human heteromyeloma cell lines for the production of human monoclonal antibodies have been described, for example, by Kozbor, 1984, J. Immunol. 133:3001-05; Brodeur et al., Monoclonal Antibody Production Techniques and Applications 51-63 (1987); and Boerner et al., 1991, J. Immunol. 147:86-95.

It is also possible to produce transgenic animals (e.g., mice) that are capable, upon immunization, of producing a full repertoire of human antibodies in the absence of endogenous immunoglobulin production. Transgenic mice that express human antibody repertoires have been used to generate high-affinity human sequence monoclonal antibodies against a wide variety of potential drug targets (see, e.g., Jakobovits, A., 1995, Curr. Opin. Biotechnol. 6(5):561-66; Brüggemann and Taussing, 1997, Curr. Opin. Biotechnol. 8(4):455-58; U.S. Pat. Nos. 6,075,181 and 6,150,584; and Lonberg et al., 2005, Nature Biotechnol. 23:1117-25).

Alternatively, the human antibody may be prepared via immortalization of human B lymphocytes producing an antibody directed against a target antigen (e.g., such B lymphocytes may be recovered from an individual or may have been immunized in vitro) (see, e.g., Cole et al., Monoclonal Antibodies and Cancer Therapy (1985); Boerner et al., 1991, J. Immunol. 147(1):86-95; and U.S. Pat. No. 5,750,373).

Gene shuffling can also be used to derive human antibodies from non-human, for example, rodent, antibodies, where the human antibody has similar affinities and specificities to the starting non-human antibody. According to this method, which is also called “epitope imprinting” or “guided selection,” either the heavy or light chain variable region of a non-human antibody fragment obtained by phage display techniques as described herein is replaced with a repertoire of human V domain genes, creating a population of non-human chain/human chain scFv or Fab chimeras. Selection with antigen results in isolation of a non-human chain/human chain chimeric scFv or Fab wherein the human chain restores the antigen binding site destroyed upon removal of the corresponding non-human chain in the primary phage display clone (e.g., the epitope guides (imprints) the choice of the human chain partner). When the process is repeated in order to replace the remaining non-human chain, a human antibody is obtained (see, e.g., PCT WO 93/06213; and Osbourn et al., 2005, Methods 36:61-68). Unlike traditional humanization of non-human antibodies by CDR grafting, this technique provides completely human antibodies, which have no FR or CDR residues of non-human origin. Examples of guided selection to humanize mouse antibodies towards cell surface antigens include the folate-binding protein present on ovarian cancer cells (see, e.g., Figini et al., 1998, Cancer Res. 58:991-96) and CD147, which is highly expressed on hepatocellular carcinoma (see, e.g., Bao et al., 2005, Cancer Biol. Ther. 4:1374-80).

A potential disadvantage of the guided selection approach is that shuffling of one antibody chain while keeping the other constant could result in epitope drift. In order to maintain the epitope recognized by the non-human antibody, CDR retention can be applied (see, e.g., Klimka et al., 2000, Br. J. Cancer. 83:252-60; and Beiboer et al., 2000, J. Mol. Biol. 296:833-49). In this method, the non-human VH CDR3 is commonly retained, as this CDR may be at the center of the antigen-binding site and may be the most important region of the antibody for antigen recognition. In some instances, however, VH CDR3 and VL CDR3, as well as VH CDR2, VL CDR2, and VL CDR1 of the non-human antibody may be retained.

### 6.3.6 Bispecific Antibodies

Bispecific antibodies are monoclonal antibodies that have binding specificities for at least two different antigens. In certain embodiments, bispecific antibodies are human or humanized antibodies. In certain embodiments, one of the binding specificities is for Sdc2 and the other is for any other antigen. In some embodiments, one of the binding specificities is for Sdc2, and the other is for another surface antigen expressed on cells expressing Sdc2. In certain embodiments, bispecific antibodies may bind to two different epitopes of Sdc2. Bispecific antibodies can be prepared as full-length antibodies or antibody fragments (e.g., F(ab′)₂ bispecific antibodies).

Methods for making bispecific antibodies are known in the art, such as, by co-expression of two immunoglobulin heavy chain-light chain pairs, where the two heavy chains have different specificities (see, e.g., Milstein and Cuello, 1983, Nature 305:537-40). For further details of generating bispecific antibodies, see, for example, Bispecific Antibodies (Kontermann ed., 2011).

### Multivalent Antibodies

A multivalent antibody may be internalized (and/or catabolized) faster than a bivalent antibody by a cell expressing an antigen to which the antibodies bind. The antibodies of the present disclosure can be multivalent antibodies (which are other than of the IgM class) with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. In certain embodiments, the dimerization domain comprises (or consists of) an Fc region or a hinge region. In this scenario, the antibody will comprise an Fc region and three or more antigen binding sites amino terminal to the Fc region. In certain embodiments, a multivalent antibody comprises (or consists of) three to about eight antigen binding sites. In one such embodiment, a multivalent antibody comprises (or consists of) four antigen binding sites. The multivalent antibody comprises at least one polypeptide chain (e.g., two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein may further comprise at least two (e.g., four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

### 6.3.7 Fc Engineering

It may be desirable to modify an anti-Sdc2 antibody provided herein by Fc engineering. In certain embodiments, the modification to the Fc region of the antibody results in the decrease or elimination of an effector function of the antibody. In certain embodiments, the effector function is ADCC, ADCP, and/or CDC. In some embodiments, the effector function is ADCC. In other embodiments, the effector function is ADCP. In other embodiments, the effector function is CDC. In one embodiment, the effector function is ADCC and ADCP. In one embodiment, the effector function is ADCC and CDC. In one embodiment, the effector function is ADCP and CDC. In one embodiment, the effector function is ADCC, ADCP and CDC. This may be achieved by introducing one or more amino acid substitutions in an Fc region of the antibody. For example, substitutions into human IgG1 using IgG2 residues at positions 233-236 and IgG4 residues at positions 327, 330, and 331 were shown to greatly reduce ADCC and CDC (see, e.g., Armour et al., 1999, Eur. J. Immunol. 29(8):2613-24; and Shields et al., 2001, J. Biol. Chem. 276(9): 6591-604). Other Fc variants are provided elsewhere herein.

To increase the serum half-life of the antibody, one may incorporate a salvage receptor binding epitope into the antibody (especially an antibody fragment), for example, as described in U.S. Pat. No. 5,739,277. Term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

### 6.3.8 Alternative Binding Agents

The present disclosure encompasses non-immunoglobulin binding agents that specifically bind to the same epitope as an anti-Sdc2 antibody disclosed herein. In some embodiments, a non-immunoglobulin binding agent is identified as an agent that displaces or is displaced by an anti-Sdc2 antibody of the present disclosure in a competitive binding assay. These alternative binding agents may include, for example, any of the engineered protein scaffolds known in the art. Such scaffolds may comprise one or more CDRs as shown in Tables 4-8. Such scaffolds include, for example, anticalins, which are based upon the lipocalin scaffold, a protein structure characterized by a rigid beta-barrel that supports four hypervariable loops which form the ligand binding site. Novel binding specificities may be engineered by targeted random mutagenesis in the loop regions, in combination with functional display and guided selection (see, e.g., Skerra, 2008, FEBS J. 275:2677-83). Other suitable scaffolds may include, for example, adnectins, or monobodies, based on the tenth extracellular domain of human fibronectin III (see, e.g., Koide and Koide, 2007, Methods Mol. Biol. 352:95-109); affibodies, based on the Z domain of staphylococcal protein A (see, e.g., Nygren et al., 2008, FEBS J. 275:2668-76); DARPins, based on ankyrin repeat proteins (see, e.g., Stumpp et al., 2008, Drug. Discov. Today 13:695-701); fynomers, based on the SH3 domain of the human Fyn protein kinase (see, e.g., Grabulovski et al., 2007, J. Biol. Chem. 282:3196-204); affitins, based on Sac7d from Sulfolobus acidolarius (see, e.g., Krehenbrink et al., 2008, J. Mol. Biol. 383:1058-68); affilins, based on human y-B-crystallin (see, e.g., Ebersbach et al., 2007, J. Mol. Biol. 372:172-85); avimers, based on the A domain of membrane receptor proteins (see, e.g., Silverman et al., 2005, Biotechnol. 23:1556-61); cysteine-rich knottin peptides (see, e.g., Kolmar, 2008, FEBS J. 275:2684-90); and engineered Kunitz-type inhibitors (see, e.g., Nixon and Wood, 2006, Curr. Opin. Drug. Discov. Dev. 9:261-68). For a review, see, for example, Gebauer and Skerra, 2009, Curr. Opin. Chem. Biol. 13:245-55.

### Antibody Variants

In some embodiments, amino acid sequence modification(s) of the antibodies that bind to Sdc2 or described herein are contemplated. For example, it may be desirable to improve the binding affinity and/or other biological properties of the antibody, including but not limited to specificity, thermostability, expression level, effector functions, glycosylation, reduced immunogenicity, or solubility. Thus, in addition to the anti-Sdc2 antibodies provided herein, it is contemplated that anti-Sdc2 antibody variants can be prepared. For example, anti-Sdc2 antibody variants can be prepared by introducing appropriate nucleotide changes into the encoding DNA, and/or by synthesis of the desired antibody or polypeptide. Those skilled in the art who appreciate that amino acid changes may alter post-translational processes of the anti-Sdc2 antibody, such as changing the number or position of glycosylation sites or altering the membrane anchoring characteristics.

In some embodiments, antibodies provided herein are chemically modified, for example, by the covalent attachment of any type of molecule to the antibody. The antibody derivatives may include antibodies that have been chemically modified, for example, by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formulation, metabolic synthesis of tunicamycin, etc. Additionally, the antibody may contain one or more non-classical amino acids.

Variations may be a substitution, deletion, or insertion of one or more codons encoding the antibody or polypeptide that results in a change in the amino acid sequence as compared with the native sequence antibody or polypeptide. Amino acid substitutions can be the result of replacing one amino acid with another amino acid having similar structural and/or chemical properties, such as the replacement of a leucine with a serine, e.g., conservative amino acid replacements. Insertions or deletions may optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the full-length or mature native sequence.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for antibody-directed enzyme prodrug therapy) or a polypeptide which increases the serum half-life of the antibody.

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Alternatively, conservative (e.g., within an amino acid group with similar properties and/or side chains) substitutions may be made, so as to maintain or not significantly change the properties. Amino acids may be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser(S), Thr (T), Cys (C), Tyr (Y), Asn (N), Gln (Q); (3) acidic: Asp (D), Glu (E); and (4) basic: Lys (K), Arg (R), His (H).

Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

Non-conservative substitutions entail exchanging a member of one of these classes for another class. Such substituted residues also may be introduced into the conservative substitution sites or, into the remaining (non-conserved) sites. Accordingly, in one embodiment, an antibody or fragment thereof that binds to a Sdc2 epitope comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of a murine monoclonal antibody provided herein. In one embodiment, an antibody or fragment thereof that binds to a Sdc2 epitope comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to an amino acid sequence depicted in Tables 1 and 3-8. In yet another embodiment, an antibody or fragment thereof that binds to a Sdc2 epitope comprises a VH CDR and/or a VL CDR amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to a VH CDR amino acid sequence depicted in Table 4-8 and/or a VL CDR amino acid sequence depicted in Table 4-8. The variations can be made using methods known in the art such as oligonucleotide-mediated (site-directed) mutagenesis, alanine scanning, and PCR mutagenesis. Site-directed mutagenesis (see, e.g., Carter, 1986, Biochem J. 237:1-7; and Zoller et al., 1982, Nucl. Acids Res. 10:6487-500), cassette mutagenesis (see, e.g., Wells et al., 1985, Gene 34:315-23), or other known techniques can be performed on the cloned DNA to produce the anti-Sdc2 antibody variant DNA.

Any cysteine residue not involved in maintaining the proper conformation of the anti-Sdc2 antibody also may be substituted, for example, with another amino acid, such as alanine or serine, to improve the oxidative stability of the molecule and to prevent aberrant crosslinking. Conversely, cysteine bond(s) may be added to the anti-Sdc2 antibody to improve its stability (e.g., where the antibody is an antibody fragment such as an Fv fragment).

In some embodiments, an anti-Sdc2 antibody molecule of the present disclosure is a “de-immunized” antibody. A “de-immunized” anti-Sdc2 antibody is an antibody derived from a humanized or chimeric anti-Sdc2 antibody, which has one or more alterations in its amino acid sequence resulting in a reduction of immunogenicity of the antibody, compared to the respective original non-de-immunized antibody. One of the procedures for generating such antibody mutants involves the identification and removal of T cell epitopes of the antibody molecule. In a first step, the immunogenicity of the antibody molecule can be determined by several methods, for example, by in vitro determination of T cell epitopes or in silico prediction of such epitopes, as known in the art. Once the critical residues for T cell epitope function have been identified, mutations can be made to remove immunogenicity and retain antibody activity. For review, see, for example, Jones et al., 2009, Methods in Molecular Biology 525:405-23.

### 6.3.9 In Vitro Affinity Maturation

In some embodiments, antibody variants having an improved property such as affinity, stability, or expression level as compared to a parent antibody may be prepared by in vitro affinity maturation. Like the natural prototype, in vitro affinity maturation is based on the principles of mutation and selection. Libraries of antibodies are displayed as Fab, scFv, or V domain fragments either on the surface of an organism (e.g., phage, bacteria, yeast, or mammalian cell) or in association (e.g., covalently or non-covalently) with their encoding mRNA or DNA. Affinity selection of the displayed antibodies allows isolation of organisms or complexes carrying the genetic information encoding the antibodies. Two or three rounds of mutation and selection using display methods such as phage display usually results in antibody fragments with affinities in the low nanomolar range. Affinity matured antibodies can have nanomolar or even picomolar affinities for the target antigen.

Phage display is a widespread method for display and selection of antibodies. The antibodies are displayed on the surface of Fd or M13 bacteriophages as fusions to the bacteriophage coat protein. Selection involves exposure to antigen to allow phage-displayed antibodies to bind their targets, a process referred to as “panning.” Phage bound to antigen are recovered and used to infect bacteria to produce phage for further rounds of selection. For review, see, for example, Hoogenboom, 2002, Methods. Mol. Biol. 178:1-37; and Bradbury and Marks, 2004, J. Immunol. Methods 290:29-49.

In a yeast display system (see, e.g., Boder et al., 1997, Nat. Biotech. 15:553-57; and Chao et al., 2006, Nat. Protocols 1:755-68), the antibody may be displayed as single-chain variable fusions (scFv) in which the heavy and light chains are connected by a flexible linker. The scFv is fused to the adhesion subunit of the yeast agglutinin protein Aga2p, which attaches to the yeast cell wall through disulfide bonds to Aga1p. Display of a protein via Aga2p projects the protein away from the cell surface, minimizing potential interactions with other molecules on the yeast cell wall. Magnetic separation and flow cytometry are used to screen the library to select for antibodies with improved affinity or stability. Binding to a soluble antigen of interest is determined by labeling of yeast with biotinylated antigen and a secondary reagent such as streptavidin conjugated to a fluorophore. Variations in surface expression of the antibody can be measured through immunofluorescence labeling of either the hemagglutinin or c-Myc epitope tag flanking the scFv. Expression has been shown to correlate with the stability of the displayed protein, and thus antibodies can be selected for improved stability as well as affinity (see, e.g., Shusta et al., 1999, J. Mol. Biol. 292:949-56). An additional advantage of yeast display is that displayed proteins are folded in the endoplasmic reticulum of the eukaryotic yeast cells, taking advantage of endoplasmic reticulum chaperones and quality-control machinery. Once maturation is complete, antibody affinity can be conveniently “titrated” while displayed on the surface of the yeast, eliminating the need for expression and purification of each clone. A theoretical limitation of yeast surface display is the potentially smaller functional library size than that of other display methods; however, a recent approach uses the yeast cells' mating system to create combinatorial diversity estimated to be 10¹⁴ in size (see, e.g., U.S. Pat. Publication 2003/0186374; and Blaise et al., 2004, Gene 342:211-18).

In ribosome display, antibody-ribosome-mRNA (ARM) complexes are generated for selection in a cell-free system. The DNA library coding for a particular library of antibodies is genetically fused to a spacer sequence lacking a stop codon. This spacer sequence, when translated, is still attached to the peptidyl tRNA and occupies the ribosomal tunnel, and thus allows the protein of interest to protrude out of the ribosome and fold. The resulting complex of mRNA, ribosome, and protein can bind to surface-bound ligand, allowing simultaneous isolation of the antibody and its encoding mRNA through affinity capture with the ligand. The ribosome-bound mRNA is then reverse transcribed back into cDNA, which can then undergo mutagenesis and be used in the next round of selection (see, e.g., Fukuda et al., 2006, Nucleic Acids Res. 34:c127). In mRNA display, a covalent bond between antibody and mRNA is established using puromycin as an adaptor molecule (Wilson et al., 2001, Proc. Natl. Acad. Sci. USA 98:3750-55).

As these methods are performed entirely in vitro, they provide two main advantages over other selection technologies. First, the diversity of the library is not limited by the transformation efficiency of bacterial cells, but only by the number of ribosomes and different mRNA molecules present in the test tube. Second, random mutations can be introduced easily after each selection round, for example, by non-proofreading polymerases, as no library must be transformed after any diversification step.

In a mammalian cell display system (see, e.g., Bowers et al., 2011, Proc Natl Acad Sci USA. 108:20455-60), a fully human library of IgGs is constructed based on germline sequence V-gene segments joined to prerecombined D (J) regions. Full-length V regions for heavy chain and light chain are assembled with human heavy chain and light chain constant regions and transfected into a mammalian cell line (e.g., HEK293). The transfected library is expanded and subjected to several rounds of negative selection against streptavidin (SA)-coupled magnetic beads, followed by a round of positive selection against SA-coupled magnetic beads coated with biotinylated target protein, peptide fragment, or epitope. Positively selected cells are expanded, and then sorted by rounds of FACS to isolate single cell clones displaying antibodies that specifically bind to the target protein, peptide fragment, or epitope. Heavy and light chain pairs from these single cell clones are retransfected with AID for further maturation. Several rounds of mammalian cell display, coupled with AID-triggered somatic hypermutation, generate high specificity, high affinity antibodies.

Diversity may also be introduced into the CDRs or the whole V genes of the antibody libraries in a targeted manner or via random introduction. The former approach includes sequentially targeting all the CDRs of an antibody via a high or low level of mutagenesis or targeting isolated hot spots of somatic hypermutations (see, e.g., Ho et al., 2005, J. Biol. Chem. 280:607-17) or residues suspected of affecting affinity on experimental basis or structural reasons. In a specific embodiment, somatic hypermutation is performed by AID-triggered somatic hypermutation, e.g., using the SHM-XEL™ platform (AnaptysBio, San Diego, CA). Random mutations can be introduced throughout the whole V gene using E. coli mutator strains, error-prone replication with DNA polymerases (see, e.g., Hawkins et al., 1992, J. Mol. Biol. 226:889-96), or RNA replicases. Diversity may also be introduced by replacement of regions that are naturally diverse via DNA shuffling or similar techniques (see, e.g., Lu et al., 2003, J. Biol. Chem. 278:43496-507; U.S. Pat. Nos. 5,565,332 and 6,989,250). Alternative techniques target hypervariable loops extending into framework-region residues (see, e.g., Bond et al., 2005, J. Mol. Biol. 348:699-709) employ loop deletions and insertions in CDRs or use hybridization-based diversification (see, e.g., U.S. Pat. Publication No. 2004/0005709). Additional methods of generating diversity in CDRs are disclosed, for example, in U.S. Pat. No. 7,985,840. Further methods that can be used to generate antibody libraries and/or antibody affinity maturation are disclosed, e.g., in U.S. Pat. Nos. 8,685,897 and 8,603,930, and U.S. Publ. Nos. 2014/0170705, 2014/0094392, 2012/0028301, 2011/0183855, and 2009/0075378, each of which are incorporated herein by reference.

Screening of the libraries can be accomplished by various techniques known in the art. For example, Sdc2 can be immobilized onto solid supports, columns, pins, or cellulose/poly(vinylidene fluoride) membranes/other filters, expressed on host cells affixed to adsorption plates or used in cell sorting, or conjugated to biotin for capture with streptavidin-coated beads or used in any other method for panning display libraries.

For review of in vitro affinity maturation methods, see, e.g., Hoogenboom, 2005, Nature Biotechnology 23:1105-16; Quiroz and Sinclair, 2010, Revista Ingeneria Biomedia 4:39-51; and references therein.

### 6.3.10 Modifications of Anti-Sdc2 Antibodies

Covalent modifications of anti-Sdc2 antibodies are included within the scope of the present disclosure. Covalent modifications include reacting targeted amino acid residues of an anti-Sdc2 antibody with an organic derivatizing agent that is capable of reacting with selected side chains or the N- or C-terminal residues of the anti-Sdc2 antibody. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (see, e.g., Creighton, Proteins: Structure and Molecular Properties 79-86 (1983)), acetylation of the N-terminal amine, and amidation of any C-terminal carboxyl group.

Other types of covalent modification of the anti-Sdc2 antibody included within the scope of this present disclosure include altering the native glycosylation pattern of the antibody or polypeptide (see, e.g., Beck et al., 2008, Curr. Pharm. Biotechnol. 9:482-501; and Walsh, 2010, Drug Discov. Today 15:773-80) and linking the antibody to one of a variety of nonproteinaceous polymers, e.g., polyethylene glycol (PEG), polypropylene glycol, or polyoxyalkylenes, in the manner set forth, for example, in U.S. Pat. Nos. 4,640,835; 4,496,689; 4,301,144; 4,670,417; 4,791,192; or 4,179,337.

An anti-Sdc2 antibody of the present disclosure may also be modified to form chimeric molecules comprising an anti-Sdc2 antibody fused to another, heterologous polypeptide or amino acid sequence, for example, an epitope tag (see, e.g., Terpe, 2003, Appl. Microbiol. Biotechnol. 60:523-33) or the Fc region of an IgG molecule (see, e.g., Aruffo, Antibody Fusion Proteins 221-42 (Chamow and Ashkenazi eds., 1999)).

Also provided herein are fusion proteins comprising an antibody provided herein that binds to a Sdc2 antigen and a heterologous polypeptide. In some embodiments, the heterologous polypeptide to which the antibody is fused is useful for targeting the antibody to cells having cell surface expressed Sdc2.

Also provided herein are panels of antibodies that bind to a Sdc2 antigen. In specific embodiments, the panels of antibodies have different association rates, different dissociation rates, different affinities for a Sdc2 antigen, and/or different specificities for a Sdc2 antigen. In some embodiments, the panels comprise or consist of about 10, about 25, about 50, about 75, about 100, about 125, about 150, about 175, about 200, about 250, about 300, about 350, about 400, about 450, about 500, about 550, about 600, about 650, about 700, about 750, about 800, about 850, about 900, about 950, or about 1000 antibodies or more. Panels of antibodies can be used, for example, in 96-well or 384-well plates, for assays such as ELISAs.

### 6.4 Nucleic Acids and Vectors Encoding Anti-Syndecan-2 Antibodies

In an embodiment, an isolated nucleic acid is provided, the isolated nucleic acid encoding a heavy chain of an anti-syndecan-2 antibody or antigen-binding fragment thereof which comprising at least 80% sequence identity to at least one amino acid of the group consisting of SEQ ID NOs: 1, 3, 5, 7, 9, 11, 13, 15, 17, 19, 21, and 23. In an embodiment, the nucleic acid encoding the heavy chain comprises at least 80%, 81%, 82%, 83%, 84%, 85%, 86%, 87%, 88%, 89%, 90%, 91%, 92% 93%, 94%, 95%, 96%, 97%, 98%, 99%, 99.5%, 99.6%, 99.7%, 99.8%, or 99.9% sequence identity, or comprises 100% sequence identity, to at least one amino acid of the group consisting of SEQ ID NOs: 1, 3, 5, 7, 9, 11, 13, 15, 17, 19, 21, and 23. In another embodiment, a nucleic acid is provided, the nucleic acid encoding a light chain of an anti-syndecan-2 antibody and comprising at least 95% sequence identity to at least one amino acid of the group consisting of SEQ ID NOs: 2, 4, 6, 8, 10, 12, 14, 16, 18, 20, 22, and 24. In another embodiment, the nucleic acid encoding the light chain comprises at least 80%, 81%, 82%, 83%, 84%, 85%, 86%, 87%, 88%, 89%, 90%, 91%, 92% 93%, 94%, 95%, 96%, 97%, 98%, 99%, 99.5%, 99.6%, 99.7%, 99.8%, or 99.9% sequence identity, or comprises 100% sequence identity, to at least one amino acid of the group consisting of SEQ ID NOs: 2, 4, 6, 8, 10, 12, 14, 16, 18, 20, 22, and 24. In an embodiment, the nucleic acids encoding the heavy chain and light chain are comprised within a single nucleic acid.

In one embodiment, at least one of the nucleic acids encoding the heavy chain and the light chain of the anti-syndecan-2 antibody is codon-optimized. In an embodiment, the codon-optimized nucleic acid is optimized for a mammal, including a human, a rabbit, a rat, a mouse, a moose, a horse, a donkey, a guinea pig, a hamster, a monkey, a great ape, a chimpanzee, a gorilla, a bonobo, a cow, a cat, a dog, a non-human primate; a bird; a reptile; a fish; an insect, including a fruit fly; a Mollusca, and other forms of vertebrates and invertebrates including Protostomia, Deuterostomia, Chordata, Ambulacraria, Lophotrochazoa, Spiralia, Ecdysozoa, Arthropoda, Tactopoda, Panarthropoda, Gnathifera, Platytrochozoa, Rouphozoa, Gastrotricha, Platyhelminthes, Mesozoa, Annelida, Krytotrochozoa, etc, and cells thereof. In an embodiment, the codon-optimized nucleic acid can be codon optimized for a single-celled organism including a protozoon, a bacterium, and an archea. Codon optimization for humans, veterinary animals (i.e., domesticated animals), and animals used in bench-side and pre-clinical models are preferred. In a preferred embodiment, the codon-optimized nucleic acid sequence is codon-optimized for a cell line or primary cells. In a further preferred embodiment, the codon-optimization is for a CHO cell.

In a preferred embodiment, the nucleic acid encoding the heavy chain and the nucleic acid encoding the light chain are introduced into the same cell, wherein the cell expresses the anti-syndecan-2 antibody or antigen-binding fragment thereof which comprises the heavy chain variable region and the light chain variable region.

Another aspect provides a vector comprising any one of the isolated nucleic acids disclosed herein. In certain embodiments, the vector is selected from the group consisting of a DNA vector, an RNA vector, a plasmid, a lentiviral vector, an adenoviral vector, an adeno-associated viral vector, and a retroviral vector. In certain embodiments, the vector is an expression vector.

Also provided is a host cell comprising any of the vectors or nucleic acids disclosed herein. The host cell may be of eukaryotic, prokaryotic, mammalian, or bacterial origin. A method of producing a binding polypeptide or scFv that binds to FAP is also provided herein, wherein the method comprises culturing the host cell.

In some embodiments, a nucleic acid of the present disclosure may be operably linked to a transcriptional control element, e.g., a promoter, and enhancer, etc. Suitable promoter and enhancer elements are known to those of skill in the art.

In certain embodiments, the nucleic acid is in operable linkage with a promoter. In certain embodiments, the promoter is a phosphoglycerate kinase-1 (PGK) promoter.

For expression in a bacterial cell, suitable promoters include, but are not limited to, lacI, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-1 promoter; and various art-known tissue specific promoters. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

For expression in a yeast cell, a suitable promoter is a constitutive promoter such as an ADH1 promoter, a PGK1 promoter, an ENO promoter, a PYK1 promoter and the like; or a regulatable promoter such as a GAL1 promoter, a GAL10 promoter, an ADH2 promoter, a PHOS promoter, a CUP1 promoter, a GALT promoter, a MET25 promoter, a MET3 promoter, a CYC1 promoter, a HIS3 promoter, an ADH1 promoter, a PGK promoter, a GAPDH promoter, an ADC1 promoter, a TRP1 promoter, a URA3 promoter, a LEU2 promoter, an ENO promoter, a TP1 promoter, and AOX1 (e.g., for use in Pichia). Selection of the appropriate vector and promoter is well within the level of ordinary skill in the art. Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a tre promoter; a tac promoter, and the like; an araBAD promoter; in vivo regulated promoters, such as an ssaG promoter or a related promoter (see, e.g., U.S. Patent Publication No. 20040131637), a pagC promoter (Pulkkinen and Miller, J. Bacteriol. (1991) 173(1): 86-93; Alpuche-Aranda et al., Proc. Natl. Acad. Sci. USA (1992) 89(21): 10079-83), a nirB promoter (Harborne et al. Mol. Micro. (1992) 6:2805-2813), and the like (see, e.g., Dunstan et al., Infect. Immun. (1999) 67:5133-5141; McKelvie et al., Vaccine (2004) 22:3243-3255; and Chatfield et al., Biotechnol. (1992) 10:888-892); a sigma70 promoter, e.g., a consensus sigma70 promoter (see, e.g., GenBank Accession Nos. AX798980, AX798961, and AX798183); a stationary phase promoter, e.g., a dps promoter, a spv promoter, and the like; a promoter derived from the pathogenicity island SPI-2 (see, e.g., WO96/17951); an actA promoter (see, e.g., Shetron-Rama et al., Infect. Immun. (2002) 70:1087-1096); a rpsM promoter (see, e.g., Valdivia and Falkow Mol. Microbiol. (1996). 22:367); a tet promoter (see, e.g., Hillen, W. and Wissmann, A. (1989) In Saenger, W. and Heinemann, U. (eds), Topics in Molecular and Structural Biology, Protein—Nucleic Acid Interaction. Macmillan, London, UK, Vol. 10, pp. 143-162); an SP6 promoter (see, e.g., Melton et al., Nucl. Acids Res. (1984) 12:7035); and the like. Suitable strong promoters for use in prokaryotes such as Escherichia coli include, but are not limited to Trc, Tac, T5, T7, and PLambda. Non-limiting examples of operators for use in bacterial host cells include a lactose promoter operator (LacI repressor protein changes conformation when contacted with lactose, thereby preventing the Lad repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator (see, e.g., deBoer et al., Proc. Natl. Acad. Sci. U.S.A. (1983) 80:21-25).

Other examples of suitable promoters include the immediate early cytomegalovirus (CMV) promoter sequence. This promoter sequence is a strong constitutive promoter sequence capable of driving high levels of expression of any polynucleotide sequence operatively linked thereto. Other constitutive promoter sequences may also be used, including, but not limited to a simian virus 40 (SV40) early promoter, a mouse mammary tumor virus (MMTV) or human immunodeficiency virus (HIV) long terminal repeat (LTR) promoter, a MoMuLV promoter, an avian leukemia virus promoter, an Epstein-Barr virus immediate early promoter, a Rous sarcoma virus promoter, the EF-1 alpha promoter, as well as human gene promoters such as, but not limited to, an actin promoter, a myosin promoter, a hemoglobin promoter, and a creatine kinase promoter. Further, the promoters provided herein should not be limited to the use of constitutive promoters. Inducible promoters are also contemplated. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

In some embodiments, the locus or construct or transgene containing the suitable promoter is irreversibly switched through the induction of an inducible system. Suitable systems for induction of an irreversible switch are well known in the art, e.g., induction of an irreversible switch may make use of a Cre-lox-mediated recombination (see, e.g., Fuhrmann-Benzakein, et al., Proc. Natl. Acad. Sci. USA (2000) 28:e99, the disclosure of which is incorporated herein by reference). Any suitable combination of recombinase, endonuclease, ligase, recombination sites, etc. known to the art may be used in generating an irreversibly switchable promoter. Methods, mechanisms, and requirements for performing site-specific recombination, described elsewhere herein, find use in generating irreversibly switched promoters and are well known in the art, see, e.g., Grindley et al. Annual Review of Biochemistry (2006) 567-605; and Tropp, Molecular Biology (2012) (Jones & Bartlett Publishers, Sudbury, Mass.), the disclosures of which are incorporated herein by reference.

A nucleic acid of the present disclosure may be present within an expression vector and/or a cloning vector. An expression vector can include a selectable marker, an origin of replication, and other features that provide for replication and/or maintenance of the vector. Suitable expression vectors include, e.g., plasmids, viral vectors, and the like. Large numbers of suitable vectors and promoters are known to those of skill in the art; many are commercially available for generating a subject recombinant construct. The following vectors are provided by way of example and should not be construed in anyway as limiting: Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g., viral vectors based on vaccinia virus; poliovirus; adenovirus (see, e.g., Li et al., Invest. Opthalmol. Vis. Sci. (1994) 35:2543-2549; Borras et al., Gene Ther. (1999) 6:515-524; Li and Davidson, Proc. Natl. Acad. Sci. USA (1995) 92:7700-7704; Sakamoto et al., H. Gene Ther. (1999) 5:1088-1097; WO 94/12649, WO 93/03769; WO 93/19191; WO 94/28938; WO 95/11984 and WO 95/00655); adeno-associated virus (see, e.g., Ali et al., Hum. Gene Ther. (1998) 9:81-86, Flannery et al., Proc. Natl. Acad. Sci. USA (1997) 94:6916-6921; Bennett et al., Invest. Opthalmol. Vis. Sci. (1997) 38:2857-2863; Jomary et al., Gene Ther. (1997) 4:683 690, Rolling et al., Hum. Gene Ther. (1999) 10:641-648; Ali et al., Hum. Mol. Genet. (1996) 5:591-594; Srivastava in WO 93/09239, Samulski et al., J. Vir. (1989) 63:3822-3828; Mendelson et al., Virol. (1988) 166:154-165; and Flotte et al., Proc. Natl. Acad. Sci. USA (1993) 90:10613-10617); SV40; herpes simplex virus; human immunodeficiency virus (see, e.g., Miyoshi et al., Proc. Natl. Acad. Sci. USA (1997) 94:10319-23; Takahashi et al., J. Virol. (1999) 73:7812-7816); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

Additional expression vectors suitable for use are, e.g., without limitation, a lentivirus vector, a gamma retrovirus vector, a foamy virus vector, an adeno-associated virus vector, an adenovirus vector, a pox virus vector, a herpes virus vector, an engineered hybrid virus vector, a transposon mediated vector, and the like. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses.

In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

In some embodiments, an expression vector may be used to introduce the nucleic acid into a host cell. Accordingly, an expression vector provided herein may comprise a nucleic acid encoding a polypeptide (e.g., an antibody or antigen-binding fragment thereof). In some embodiments, the expression vector will comprise additional elements that will aid in the functional expression of the polypeptide encoded therein. In some embodiments, an expression vector comprising a nucleic acid encoding for a polypeptide further comprises a mammalian promoter. In one embodiment, the vector further comprises an elongation-factor-1-alpha promoter (EF-1α promoter). Use of an EF-1α promoter may increase the efficiency in expression of downstream transgenes. Physiologic promoters (e.g., an EF-1α promoter) may be less likely to induce integration mediated genotoxicity and may abrogate the ability of the retroviral vector to transform stem cells. Other physiological promoters suitable for use in a vector (e.g., lentiviral vector) are known to those of skill in the art and may be incorporated into a vector provided herein. In some embodiments, the vector (e.g., lentiviral vector) further comprises a non-requisite cis acting sequence that may improve titers and gene expression. One non-limiting example of a non-requisite cis acting sequence is the central polypurine tract and central termination sequence (cPPT/CTS) which is important for efficient reverse transcription and nuclear import. Other non-requisite cis acting sequences are known to those of skill in the art and may be incorporated into a vector (e.g., lentiviral vector) provided herein. In some embodiments, the vector further comprises a posttranscriptional regulatory element. Posttranscriptional regulatory elements may improve RNA translation, improve transgene expression and stabilize RNA transcripts. One example of a posttranscriptional regulatory element is the woodchuck hepatitis virus posttranscriptional regulatory element (WPRE). Accordingly, in some embodiments a vector for the present invention further comprises a WPRE sequence. Various posttranscriptional regulator elements are known to those of skill in the art and may be incorporated into a vector (e.g., lentiviral vector) provided herein. A vector provided herein may further comprise additional elements such as a rev response element (RRE) for RNA transport, packaging sequences, and 5′ and 3′ long terminal repeats (LTRs). The term “long terminal repeat” or “LTR” refers to domains of base pairs located at the ends of retroviral DNAs which comprise U3, R and U5 regions. LTRs generally provide functions required for the expression of retroviral genes (e.g., promotion, initiation and polyadenylation of gene transcripts) and to viral replication. In one embodiment, a vector (e.g., lentiviral vector) provided herein includes a 3′ U3 deleted LTR. Accordingly, a vector (e.g., lentiviral vector) provided herein may comprise any combination of the elements described herein to enhance the efficiency of functional expression of transgenes. For example, a vector (e.g., lentiviral vector) provided herein may comprise a WPRE sequence, cPPT sequence, RRE sequence, 5′LTR, 3′ U3 deleted LTR′ in addition to a nucleic acid encoding for a CAR.

Vectors provided herein may be self-inactivating vectors. As used herein, the term “self-inactivating vector” refers to vectors in which the 3′ LTR enhancer promoter region (U3 region) has been modified (e.g., by deletion or substitution). A self-inactivating vector may prevent viral transcription beyond the first round of viral replication. Consequently, a self-inactivating vector may be capable of infecting and then integrating into a host genome (e.g., a mammalian genome) only once, and cannot be passed further. Accordingly, self-inactivating vectors may greatly reduce the risk of creating a replication-competent virus.

In some embodiments, a nucleic acid provided herein may be RNA, e.g., in vitro synthesized RNA. Methods for in vitro synthesis of RNA are known to those of skill in the art; any known method can be used to synthesize RNA comprising a sequence encoding a polypeptide of the present disclosure. Methods for introducing RNA into a host cell are known in the art. See, e.g., Zhao et al. Cancer Res. (2010) 15:9053. Introducing RNA comprising a nucleotide sequence encoding a polypeptide of the present disclosure into a host cell can be carried out in vitro, ex vivo or in vivo. For example, a host cell (e.g., an NK cell, a cytotoxic T lymphocyte, etc.) can be electroporated in vitro or ex vivo with RNA comprising a nucleotide sequence encoding a polypeptide of the present disclosure.

In order to assess the expression of a polypeptide or portions thereof, the expression vector to be introduced into a cell may also contain either a selectable marker gene or a reporter gene, or both, to facilitate identification and selection of expressing cells from the population of cells sought to be transfected or infected through viral vectors. In some embodiments, the selectable marker may be carried on a separate piece of DNA and used in a co-transfection procedure. Both selectable markers and reporter genes may be flanked with appropriate regulatory sequences to enable expression in the host cells. Useful selectable markers include, without limitation, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA has been introduced into the recipient cells. Suitable reporter genes may include, without limitation, genes encoding luciferase, beta-galactosidase, chloramphenicol acetyl transferase, secreted alkaline phosphatase, or the green fluorescent protein gene (e.g., Ui-Tci et al., 2000 FEBS Letters 479:79-82).

In some embodiments, an isolated nucleic acid of the present disclosure is provided, for example, for the production of an antibody or antigen-binding fragment as described herein, e.g., in a host cell. In some embodiments, a nucleic acid of the present disclosure provides for amplification of the polypeptide-encoding nucleic acid.

Also provided is a nucleic acid encoding an antibody provided herein. In another general aspect, provide is a vector comprising an isolated nucleic acid encoding an antibody provided herein. In another general aspect, provided is a vector comprising an isolated nucleic acid encoding an antibody provided herein. Also provided is a vector comprising a nucleic acid encoding an antibody provided herein. Also provided is a host cell comprising a vector comprising a nucleic acid encoding an antibody provided herein. Also provided is a kit comprising the vector comprising a nucleic acid encoding an antibody provided herein, and packaging for the same. In another general aspect, provided herein is an isolated nucleic acid encoding a monoclonal antibody or antigen-binding fragment thereof provided herein. In certain embodiments, the antibody is an anti-Sdc2 antibody.

In one aspect, provided is a nucleic acid encoding an anti-Sdc2 antibody provided herein. In another aspect, provided is a vector comprising a nucleic acid encoding an anti-Sdc2 antibody provided herein. In one aspect, provided is a host cell comprising a vector comprising a nucleic acid encoding an anti-Sdc2 antibody provided herein. In another aspect, provided is a kit comprising a vector comprising a nucleic acid encoding an anti-Sdc2 antibody provided herein. In yet another aspect, provided is a kit comprising an antibody provided herein. In certain embodiments, the kit further comprises a container. In certain embodiments, the kit further comprises packaging. In certain embodiments, the kit further comprises instructions for use.

In certain embodiments, a nucleic acid encoding a Sdc2 antibody provided herein can be administered to a subject in a method of treatment. For example, in certain embodiments, a nucleic acid encoding a Sdc2 antibody provided herein can be used for gene therapy of a subject, for example, a human subject having a Sdc2 associated disease or disorder as provided herein.

It will be appreciated by those skilled in the art that the coding sequence of a protein can be changed (e.g., replaced, deleted, inserted, etc.) without changing the amino acid sequence of the protein. Accordingly, it will be understood by those skilled in the art that nucleic acid sequences encoding antibodies provided herein can be altered without changing the amino acid sequences of the proteins.

Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible or repressible promoter. A number of expression vectors capable of delivering nucleic acids to a cell are known in the art and can be used herein for production of an antibody or antigen-binding fragment thereof in the cell. Conventional cloning techniques or artificial gene synthesis can be used to generate a recombinant expression vector according to certain embodiments. Such techniques are well known to those skilled in the art in view of the present disclosure.

Also provided is a host cell comprising an isolated nucleic acid encoding an antibody provided herein. Also provided is a host cell comprising an isolated nucleic acid encoding an antigen binding fragment provided herein. Any host cell known to those skilled in the art in view of the present disclosure can be used for recombinant expression of antibodies or antigen-binding fragments thereof provided herein. In some embodiments, the host cells are E. coli TG1 or BL21 cells (for expression of, e.g., an scFv or Fab antibody), CHO-DG44 or CHO-K1 cells or HEK293 cells (for expression of, e.g., a full-length IgG antibody). According to particular embodiments, the recombinant expression vector is transformed into host cells by conventional methods such as chemical transfection, heat shock, or electroporation, where it is stably integrated into the host cell genome such that the recombinant nucleic acid is effectively expressed.

Also provided are methods of producing an antibody disclosed herein. The methods comprise culturing a cell comprising a nucleic acid encoding the antibody under conditions to produce an antibody and recovering the antibody from the cell or cell culture (e.g., from the supernatant). Expressed antibodies can be harvested from the cells and purified according to conventional techniques known in the art and as described herein.

### 6.5 Preparation of Anti-Sdc2 Antibodies

Anti-Sdc2 antibodies may be produced by culturing cells transformed or transfected with a vector containing anti-Sdc2 antibody-encoding nucleic acids. Polynucleotide sequences encoding polypeptide components of the antibody of the present disclosure can be obtained using standard recombinant techniques. Desired polynucleotide sequences may be isolated and sequenced from antibody producing cells such as hybridomas cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in host cells. Many vectors that are available and known in the art can be used for the purpose of the present disclosure. Selection of an appropriate vector will depend mainly on the size of the nucleic acids to be inserted into the vector and the particular host cell to be transformed with the vector. Host cells suitable for expressing antibodies of the present disclosure include prokaryotes such as Archaebacteria and Eubacteria, including Gram-negative or Gram-positive organisms, eukaryotic microbes such as filamentous fungi or yeast, invertebrate cells such as insect or plant cells, and vertebrate cells such as mammalian host cell lines. Host cells are transformed with the above-described expression vectors and cultured in conventional nutrient media modified as appropriate for inducing promoters, selecting transformants, or amplifying the genes encoding the desired sequences. Antibodies produced by the host cells are purified using standard protein purification methods as known in the art.

Methods for antibody production including vector construction, expression, and purification are further described in Plückthun et al., Antibody Engineering: Producing antibodies in Escherichia coli: From PCR to fermentation 203-52 (McCafferty et al. eds., 1996); Kwong and Rader, E. coli Expression and Purification of Fab Antibody Fragments, in Current Protocols in Protein Science (2009); Tachibana and Takekoshi, Production of Antibody Fab Fragments in Escherichia coli, in Antibody Expression and Production (Al-Rubeai ed., 2011); and Therapeutic Monoclonal Antibodies: From Bench to Clinic (An ed., 2009).

It is, of course, contemplated that alternative methods, which are well known in the art, may be employed to prepare anti-Sdc2 antibodies. For instance, the appropriate amino acid sequence, or portions thereof, may be produced by direct peptide synthesis using solid-phase techniques (see, e.g., Stewart et al., Solid-Phase Peptide Synthesis (1969); and Merrifield, 1963, J. Am. Chem. Soc. 85:2149-54). In vitro protein synthesis may be performed using manual techniques or by automation. Various portions of the anti-Sdc2 antibody may be chemically synthesized separately and combined using chemical or enzymatic methods to produce the desired anti-Sdc2 antibody. Alternatively, antibodies may be purified from cells or bodily fluids, such as milk, of a transgenic animal engineered to express the antibody, as disclosed, for example, in U.S. Pat. Nos. 5,545,807 and 5,827,690.

### 6.6 Immunoconjugates

The present disclosure also provides conjugates comprising any one of the anti-Sdc2 antibodies of the present disclosure covalently bound by a synthetic linker to one or more non-antibody agents.

In some embodiments, antibodies provided herein are conjugated or recombinantly fused, e.g., to a diagnostic or detectable molecule. The conjugated or recombinantly fused antibodies can be useful, for example, for monitoring or prognosing the onset, development, progression, and/or severity of a Sdc2-mediated disease.

Such diagnosis and detection can be accomplished, for example, by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin or avidin/biotin; fluorescent materials, such as, but not limited to, umbelliferone, fluorescein, fluorescein isothiocynate, rhodamine, dichlorotriazinylamine fluorescein, dansyl chloride, or phycoerythrin; luminescent materials, such as, but not limited to, luminol; bioluminescent materials, such as, but not limited to, luciferase, luciferin, or aequorin; chemiluminescent material, such as, but not limited to, an acridinium based compound or a HALOTAG; radioactive materials, such as, but not limited to, iodine (¹³¹I, ¹²⁵I, ¹²³I, and ¹²¹I,), carbon (¹⁴C), sulfur (³⁵S), tritium (³H), indium (¹¹⁵In, ¹¹³In, ¹¹²In, and ¹¹¹In), technetium (⁹⁹Tc), thallium (²⁰¹Ti), gallium (⁶⁸Ga and ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, or ¹¹⁷Sn; positron emitting metals using various positron emission tomographies; and non-radioactive paramagnetic metal ions.

Also described herein are antibodies that are recombinantly fused or chemically conjugated (covalent or non-covalent conjugations) to a heterologous protein or polypeptide (or fragment thereof, for example, to a polypeptide of about 10, about 20, about 30, about 40, about 50, about 60, about 70, about 80, about 90, or about 100 amino acids) to generate fusion proteins, as well as uses thereof. In particular, provided herein are fusion proteins comprising an antigen-binding fragment of an antibody provided herein (e.g., a Fab fragment, Fc fragment, Fv fragment, F (ab) 2 fragment, a VH domain, a VH CDR, a VL domain, or a VL CDR) and a heterologous protein, polypeptide, or peptide. In one embodiment, the heterologous protein, polypeptide, or peptide that the antibody is fused to is useful for targeting the antibody to a particular cell type, such as a cell that expresses Sdc2. For example, an antibody that binds to a cell surface receptor expressed by a particular cell type may be fused or conjugated to a modified antibody provided herein.

Moreover, antibodies provided herein can be fused to marker or “tag” sequences, such as a peptide, to facilitate purification. In specific embodiments, the marker or tag amino acid sequence is a hexa-histidine peptide, such as the tag provided in a pQE vector (see, e.g., QIAGEN, Inc.), among others, many of which are commercially available. For example, as described in Gentz et al., 1989, Proc. Natl. Acad. Sci. USA 86:821-24, hexa-histidine provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767-78), and the “FLAG” tag.

Methods for fusing or conjugating moieties (including polypeptides) to antibodies are known (see, e.g., Arnon et al., Monoclonal Antibodies for Immunotargeting of Drugs in Cancer Therapy, in Monoclonal Antibodies and Cancer Therapy 243-56 (Reisfeld et al. eds., 1985); Hellstrom et al., Antibodies for Drug Delivery, in Controlled Drug Delivery 623-53 (Robinson et al. eds., 2d ed. 1987); Thorpe, Antibody Carriers of Cytotoxic Agents in Cancer Therapy: A Review, in Monoclonal Antibodies: Biological and Clinical Applications 475-506 (Pinchera et al. eds., 1985); Analysis, Results, and Future Prospective of the Therapeutic Use of Radiolabeled Antibody in Cancer Therapy, in Monoclonal Antibodies for Cancer Detection and Therapy 303-16 (Baldwin et al. eds., 1985); Thorpe et al., 1982, Immunol. Rev. 62:119-58; U.S. Pat. Nos. 5,336,603; 5,622,929; 5,359,046; 5,349,053; 5,447,851; 5,723,125; 5,783,181; 5,908,626; 5,844,095; and 5,112,946; EP 307,434; EP 367,166; EP 394,827; PCT publications WO 91/06570, WO 96/04388, WO 96/22024, WO 97/34631, and WO 99/04813; Ashkenazi et al., 1991, Proc. Natl. Acad. Sci. USA, 88:10535-39; Traunecker et al., 1988, Nature, 331:84-86; Zheng et al., 1995, J. Immunol. 154:5590-600; and Vil et al., 1992, Proc. Natl. Acad. Sci. USA 89:11337-41).

Fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of anti-Sdc2 antibodies as provided herein, including, for example, antibodies with higher affinities and lower dissociation rates (see, e.g., U.S. Pat. Nos. 5,605,793; 5,811,238; 5,830,721; 5,834,252; and U.S. Pat. No. 5,837,458; Patten et al., 1997, Curr. Opinion Biotechnol. 8:724-33; Harayama, 1998, Trends Biotechnol. 16(2):76-82; Hansson et al., 1999, J. Mol. Biol. 287:265-76; and Lorenzo and Blasco, 1998, Biotechniques 24(2):308-13). Antibodies, or the encoded antibodies, may be altered by being subjected to random mutagenesis by error-prone PCR, random nucleotide insertion, or other methods prior to recombination. A polynucleotide encoding an antibody provided herein may be recombined with one or more components, motifs, sections, parts, domains, fragments, etc. of one or more heterologous molecules.

An antibody provided herein can also be conjugated to a second antibody to form an antibody heteroconjugate as described, for example, in U.S. Pat. No. 4,676,980.

Antibodies that bind to Sdc2 as provided herein may also be attached to solid supports, which are particularly useful for immunoassays or purification of the target antigen. Such solid supports include, but are not limited to, glass, cellulose, polyacrylamide, nylon, polystyrene, polyvinyl chloride, or polypropylene.

The linker may be a “cleavable linker” facilitating release of the conjugated agent in the cell, but non-cleavable linkers are also contemplated herein. Linkers for use in the conjugates of the present disclosure include, without limitation, acid labile linkers (e.g., hydrazone linkers), disulfide-containing linkers, peptidase-sensitive linkers (e.g., peptide linkers comprising amino acids, for example, valine and/or citrulline such as citrulline-valine or phenylalanine-lysine), photolabile linkers, dimethyl linkers (see, e.g., Chari et al., 1992, Cancer Res. 52:127-31; and U.S. Pat. No. 5,208,020), thioether linkers, or hydrophilic linkers designed to evade multidrug transporter-mediated resistance (see, e.g., Kovtun et al., 2010, Cancer Res. 70:2528-37).

Conjugates of the antibody and agent may be made using a variety of bifunctional protein coupling agents such as BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, sulfo-SMPB, and SVSB (succinimidyl-(4-vinylsulfone)benzoate). The present disclosure further contemplates that conjugates of antibodies and agents may be prepared using any suitable methods as disclosed in the art (see, e.g., Bioconjugate Techniques (Hermanson ed., 2d ed. 2008)).

Conventional conjugation strategies for antibodies and agents have been based on random conjugation chemistries involving the &-amino group of Lys residues or the thiol group of Cys residues, which results in heterogenous conjugates. Recently developed techniques allow site-specific conjugation to antibodies, resulting in homogeneous loading and avoiding conjugate subpopulations with altered antigen-binding or pharmacokinetics. These include engineering of “thiomabs” comprising cysteine substitutions at positions on the heavy and light chains that provide reactive thiol groups and do not disrupt immunoglobulin folding and assembly or alter antigen binding (see, e.g., Junutula et al., 2008, J. Immunol. Meth. 332:41-52; and Junutula et al., 2008, Nature Biotechnol. 26:925-32). In another method, selenocysteine is cotranslationally inserted into an antibody sequence by recoding the stop codon UGA from termination to selenocysteine insertion, allowing site specific covalent conjugation at the nucleophilic selenol group of selenocysteine in the presence of the other natural amino acids (see, e.g., Hofer et al., 2008, Proc. Natl. Acad. Sci. USA 105:12451-56; and Hofer et al., 2009, Biochemistry 48(50):12047-57).

### 6.7 Methods of Using the Antibodies and Compositions Thereof

As noted above, also provided herein are methods of using a syndecan-2 disrupting agent. In one aspect, a method of treating acute respiratory distress syndrome (ARDS) in a subject in need thereof is provided; the method comprising administering an effective amount of the anti-syndecan-2 antibody or antigen binding fragment thereof to treat the ARDS. In various embodiments the ARDS is coronavirus disease 2019-induced ARDS (COVID-19-induced ARDS). In various embodiments, the ARDS is caused by the severe acute respiratory syndrome coronavirus 2 (SARS-CoV-2), which is the coronavirus responsible for the COVID-19 disease. In various embodiments, the ARDS is caused by the subject's immune response to the infection of the SARS-CoV-2. In still further various embodiments, the ARDS is caused by the combination of the SARS-CoV-2 and the subject's immune response to the infection of the SARS-CoV-2. In various embodiments, the compositions comprising the anti-syndecan-2 antibody and/or the syndecan-2 disrupting peptide further comprises a pharmaceutically acceptable carrier.

In another aspect, provided is a method of treating a syndecan-2 associated disease in a subject in need thereof, the method comprising administering to the subject an effective amount of an anti-syndecan-2 antibody or antigen binding fragment thereof to treat the ARDS, the anti-syndecan-2 antibody or antigen-binding fragment thereof comprising a heavy chain variable region and a light chain variable region, wherein the heavy chain variable region comprises a first amino acid sequence having at least 80% sequence identity to at least one selected from the group consisting of SEQ ID NOs: 1, 3, 5, 7, 9, 11, 13, 15, 17, 19, 21, and 23 and the light chain variable region comprises a second amino acid sequence having at least 80% sequence identity to at least one selected from the group consisting of SEQ ID NOs: 2, 4, 6, 8, 10, 12, 14, 16, 18, 20, 22, and 24.

In various embodiments, the term “syndecan-2 associated disease” as used herein refers to a disease whose pathogenesis is associated with vascular leakage and/or edema formation which is mediated or regulated by the function of syndecan-2. Non-limiting examples of diseases with significant vascular leakage aspects include acute respiratory distress syndrome (ARDS), stroke (including but not limited to hemorrhagic stroke and ischemic stroke) (Fagan, et al. (2004) Stroke. 35:2220-2225), neurological diseases in which the blood brain barrier (BBB) is altered or disrupted, including but not limited to Parkinson's Diseases, Alzheimer's disease, Huntington's Disease, peripheral neuropathies, traumatic brain injury, epilepsy and multiple sclerosis, neovascular eye diseases including but not limited to wet AMD, Proliferative Diabetic Retinopathy, cardiovascular disease with a component of vascular hyperpermeability including but not limited to myocardial infarction and congestive heart failure (García-Román, et al. (2013) Cancer Letters July 28; 335(2):259-69). In various embodiments, the syndecan-2 associated disease is selected from the group consisting of blunt trauma injuries including traumatic brain injuries and battlefield injuries, peripheral vascular disease, lymphedema and inflammation-associated edema and syndromes specifically associated with edema formation including but not limited to POEMS syndrome, capillary leak syndrome (pediatric and adult) and hydrocephalus. In various embodiments, the syndecan-2 associated disease is any condition or situation that requires acceleration of healing when speed is essential (by way of non-limiting example, recovery from sport injuries in professional sports). In various embodiments, the syndecan-2 associated disease is coronavirus disease 2019-induced ARDS (COVID-19-induced ARDS). In various embodiments, the syndecan-2 associated disease is an oncological disease such as cancer. In cancer, vascular permeability can affect therapy responsiveness and disease prognosis. For example, vascularity and permeability of blood vessels feeding tumor tissue, can affect metastatic potential and tumor growth. Likewise, access of cytotoxic therapies to tumor cells can be regulated by the permeability of vessels in the tumor tissue. In certain embodiments, the anti-syndecan 2 antibodies or antigen fragments thereof can be administered in order to prevent primary tumor growth, or preventing lymphogenic or hematological metastasis, or be used alternatively in association with other therapeutic agents in order to increase the efficacy cytotoxic therapy by enhancing their ability to exit the tumor vasculature and contact tumor cells. Non-limiting examples of such therapeutic agents include, cytotoxic drugs, small molecule inhibitors, and biologic agents such as antibodies, antibody-drug conjugates, antigen-binding fragments, and the like.

In one embodiment, the subject is a mammal. In a preferred embodiment, the subject is a human.

The functional activity of antibodies provided herein can be characterized by methods known in the art and as described herein. Methods for characterizing antibodies and antigen-binding fragments thereof include, but are not limited to, affinity and specificity assays including Biacore, ELISA, and OctetRed analysis; binding assays to detect the binding of antibodies to target cells by FACS; binding assays to detect the binding of antibodies to the target antigen on cells. According to particular embodiments, the methods for characterizing antibodies and antigen-binding fragments thereof include those described below. In certain embodiments, the antibody is an anti-Sdc2 antibody.

In one aspect provided is a method of reducing vascular cell permeability, comprising contacting the vascular cells with an anti-Sdc2 antibody provided herein. In one aspect, provided is a method of reducing endothelial cell permeability, comprising contacting the endothelial cells with an anti-Sdc2 antibody provided herein.

In one aspect, provided is a method of reducing VEGFA-induced endothelial cell permeability, comprising contacting the endothelial cells with an anti-Sdc2 antibody provided herein, either before, during or after the endothelial cells are contacted with the VEGFA. In one embodiment, the endothelial cells are contacted with the anti-Sdc2 antibody before contact with the VEGFA. In one embodiment, the endothelial cells are contacted with the anti-Sdc2 antibody during contact with the VEGFA. In one embodiment, the endothelial cells are contacted with the anti-Sdc2 antibody after contact with the VEGFA.

In one aspect, provided is a method reducing vascular permeability in a subject, comprising administering to the subject an effective amount of an anti-Sdc2 antibody provided herein. In one aspect, provided is a method reducing vascular leakage in a subject, comprising administering to the subject an effective amount of an anti-Sdc2 antibody provided herein. In one aspect, provided is a method reducing endothelial permeability in a subject, comprising administering to the subject an effective amount of an anti-Sdc2 antibody provided herein. In certain embodiments of the methods provided herein, the subject has a disease caused all or in part by cells expressing Sdc2.

In another aspect, provided herein is a method of preventing, treating, or modulating a disease caused all or in part by cells expressing Sdc2, comprising administering to the subject an effective amount of an anti-Sdc2 antibody provided herein. In some embodiments, the cells are endothelial cells. In some embodiments, the cells are neural cells.

In certain embodiments of the various methods provided herein, the disease is associated with vascular permeability or vascular leakage. In one embodiment, the disease is associated with vascular permeability. In one embodiment, the disease is associated with vascular permeability. In one embodiment, the disease is an acute respiratory distress syndrome (ARDS). In one embodiment, the disease is a COVID-19-induced ARDS. In one embodiment, the disease is a hemorrhagic stroke. In one embodiment, the disease is an ischemic stroke. In one embodiment, the disease is a neurological disease in which the BBB is altered or disrupted. In one embodiment, the disease is Parkinson's Disease. In one embodiment, the disease is Alzheimer's disease. In one embodiment, the disease is Huntington's Disease. In one embodiment, the disease is a peripheral neuropathy. In one embodiment, the disease is a traumatic brain injury. In one embodiment, the disease is epilepsy. In one embodiment, the disease is multiple sclerosis. In one embodiment, the disease is a neovascular eye disease. In one embodiment, the disease is a cardiovascular disease. In one embodiment, the disease is a myocardial infarction. In one embodiment, the disease is congestive heart failure. In one embodiment, the disease is a blunt trauma injury. In one embodiment, the disease is a peripheral vascular disease. In one embodiment, the disease is a lymphedema. In one embodiment, the disease is POEMS Syndrome. In one embodiment, the disease is a pediatric capillary leak syndrome. In one embodiment, the disease is an adult capillary leak syndrome. In one embodiment, the disease is a hydrocephalus. In one embodiment, the disease is a lymphedema. In one embodiment, the disease is an inflammation-associated edema. In one embodiment, the disease is an inflammatory disease. In one embodiment, the disease is systemic lupus erythematosus. In one embodiment, the disease is a rheumatoid arthritis cardiovascular disease. In one embodiment, the disease is a neovascular eye disease. In one embodiment, the disease is AMD. In one embodiment, the disease is diabetic retinopathy. In one embodiment, the disease is a stroke. In one embodiment, the disease is an ischemic stroke. In one embodiment, the disease is a hemorrhagic stroke. In one embodiment, the disease is or a cancer.

In certain embodiments, the subject is a human. In some embodiments, the subject is a human subject in need thereof.

Also provided herein is a method of treating a disease or disorder in a subject, comprising administering to the subject an anti-Sdc2 antibody provided herein. Also provided herein is a method of treating a disease or disorder in a subject, comprising administering to the subject a Sdc2 antigen binding fragment provided herein. Also provided herein is a method of treating a disease or disorder in a subject, comprising administering to the subject a pharmaceutical composition comprising an anti-Sdc2 antibody provided herein. Also provided herein is a method of treating a disease or disorder in a subject, comprising administering to the subject a pharmaceutical composition comprising a Sdc2 antigen binding fragment provided herein.

In some embodiments, the subject is a subject in need thereof. In some embodiments, the subject is a human. In specific embodiments, the subject is administered an effective amount.

As used herein, the term “effective amount” refers to an amount of an active ingredient or component that elicits the desired biological or medicinal response in a subject.

According to particular embodiments, an effective amount refers to the amount of therapy which is sufficient to achieve one, two, three, four, or more of the following effects: reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

The effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

In some embodiments, an anti-Sdc2 antibody provided herein is used in combination with a supplemental therapy.

As used herein, the term “in combination,” in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

In one aspect provided is a method of reducing vascular cell permeability, comprising contacting the vascular cells with an Sdc2 antibody provided herein. In one aspect, provided is a method of reducing endothelial cell permeability, comprising contacting the endothelial cells with an Sdc2 antibody provided herein.

In one aspect, provided is a method of reducing VEGFA-induced endothelial cell permeability, comprising contacting the endothelial cells with an Sdc2 antibody provided herein, either before, during or after the endothelial cells are contacted with the VEGFA. In one embodiment, the endothelial cells are contacted with the Sdc2 antibody before contact with the VEGFA. In one embodiment, the endothelial cells are contacted with the Sdc2 antibody during contact with the VEGFA. In one embodiment, the endothelial cells are contacted with the Sdc2 antibody after contact with the VEGFA.

In one aspect, provided is a method reducing vascular permeability in a subject, comprising administering to the subject an effective amount of an Sdc2 antibody provided herein. In one aspect, provided is a method reducing vascular leakage in a subject, comprising administering to the subject an effective amount of an Sdc2 antibody provided herein. In one aspect, provided is a method reducing endothelial permeability in a subject, comprising administering to the subject an effective amount of an Sdc2 antibody provided herein. In certain embodiments of the methods provided herein, the subject has a disease caused all or in part by cells expressing Sdc2.

In a specific embodiment, the subject is a human. In certain embodiments, the subject is a subject in need thereof. In a specific embodiment, the subject is a human subject in need thereof.

In another aspect, provided herein is a method of preventing, treating, or modulating a disease caused all or in part by cells expressing Sdc2, comprising administering to the subject an effective amount of an Sdc2 antibody provided herein. In some embodiments, the cells are endothelial cells. In some embodiments, the cells are neural cells.

In certain embodiments of the various methods provided herein, the disease is associated with vascular permeability or vascular leakage. In one embodiment, the disease is associated with vascular permeability. In one embodiment, the disease is associated with vascular permeability. In one embodiment, the disease is an acute respiratory distress syndrome (ARDS). In one embodiment, the disease is a COVID-19-induced ARDS. In one embodiment, the disease is a hemorrhagic stroke. In one embodiment, the disease is an ischemic stroke. In one embodiment, the disease is a neurological disease in which the BBB is altered or disrupted. In one embodiment, the disease is Parkinson's Disease. In one embodiment, the disease is Alzheimer's disease. In one embodiment, the disease is Huntington's Disease. In one embodiment, the disease is a peripheral neuropathy. In one embodiment, the disease is a traumatic brain injury. In one embodiment, the disease is epilepsy. In one embodiment, the disease is multiple sclerosis. In one embodiment, the disease is a neovascular eye disease. In one embodiment, the disease is a cardiovascular disease. In one embodiment, the disease is a myocardial infarction. In one embodiment, the disease is congestive heart failure. In one embodiment, the disease is a blunt trauma injury. In one embodiment, the disease is a peripheral vascular disease. In one embodiment, the disease is a lymphedema. In one embodiment, the disease is POEMS Syndrome. In one embodiment, the disease is a pediatric capillary leak syndrome. In one embodiment, the disease is an adult capillary leak syndrome. In one embodiment, the disease is a hydrocephalus. In one embodiment, the disease is a lymphedema. In one embodiment, the disease is an inflammation-associated edema. In one embodiment, the disease is an inflammatory disease. In one embodiment, the disease is systemic lupus erythematosus. In one embodiment, the disease is a rheumatoid arthritis cardiovascular disease. In one embodiment, the disease is a neovascular eye disease. In one embodiment, the disease is AMD. In one embodiment, the disease is diabetic retinopathy. In one embodiment, the disease is a stroke. In one embodiment, the disease is an ischemic stroke. In one embodiment, the disease is a hemorrhagic stroke. In one embodiment, the disease is a cancer.

In one aspect, provided herein is a method for treating ischemic stroke comprising administering to a subject an effective amount of a Sdc2 antibody provided herein.

In one embodiment, the antibody specifically binds to Sdc2. In one embodiment, the Sdc2 is present on the surface of an endothelial cell. In one embodiment, the Sdc2 is present on the surface of a neural cell.

### 6.7.1 Methods of Managing, Preventing or Treating Stroke

In one embodiment, provided is a method of treating a stroke in a subject, comprising administering to the subject an effective amount of a Sdc2 antibody provided herein. In one embodiment, provided is a method of treating a hemorrhagic stroke in a subject, comprising administering to the subject an effective amount of a Sdc2 antibody provided herein. In one embodiment, provided is a method of treating an ischemic stroke in a subject, comprising administering to the subject an effective amount of a Sdc2 antibody provided herein. In some embodiments of the methods for treating ischemic stroke in a subject as described herein, the subject has a brain lesion area associated with the ischemic stroke.

In one aspect, provided are methods of managing, preventing, or treating ischemic stroke in a subject, comprising administering to a subject a therapeutically effective amount of an antibody that binds to a Sdc2 polypeptide, a Sdc2 polypeptide fragment, a Sdc2 peptide, or a Sdc2 epitope.

In certain embodiments of the methods, the antibodies provided herein bind to human and/or cyno Sdc2. In one embodiment, the Sdc2 antibodies bind to human Sdc2. In one embodiment, the Sdc2 antibodies bind to cyno Sdc2. In other embodiments, the antibodies bind to rodent Sdc2 (e.g., a mouse Sdc2). In other embodiments, the antibodies bind to porcine Sdc2 (e.g., a pig Sdc2). In another embodiment, the antibody binds to human Sdc2 and cyno Sdc2. In some embodiments, the antibody binds to a rodent Sdc2 (e.g., a mouse Sdc2). In some embodiments, the antibody binds to a porcine Sdc2 (e.g., a pig Sdc2). In certain embodiments, the antibodies bind to human and/or cyno Sdc2 but does not bind to rodent Sdc2. In some embodiments, the antibody or antigen-binding fragment binds specifically to an epitope which is shared by human, mouse, and porcine Sdc2 proteins or any combination thereof (i.e., human and mouse, mouse and porcine, or porcine and human).

In some embodiments, the anti-Sdc2 antibodies inhibits the binding of Dep-1 to a Sdc2 polypeptide. In some embodiments, the anti-Sdc2 antibodies competes with Dep-1 for binding to a Sdc2 polypeptide. In some embodiments, the anti-Sdc2 antibody or antigen binding fragment thereof binds to a region in the extracellular domain of Sdc2 that correspond to amino acids 123 to 140 of human Sdc2 (SEQ ID NO:25).

In some embodiments of the methods, the anti-Sdc2 antibodies bind to the extracellular domain (ECD) of Sdc2. In certain embodiments, the anti-Sdc2 antibodies bind to an epitope in the ECD of Sdc2. In certain embodiments, the anti-Sdc2 antibodies bind to an epitope in the ECD of Sdc2 to which Dep-1 binds. In some embodiments, the anti-Sdc2 antibodies compete with Dep-1 for binding to a Sdc2 polypeptide. In some embodiments, the anti-Sdc2 antibodies inhibits binding of Dep-1 to a Sdc2 polypeptide. In some embodiments, the anti-Sdc2 antibody blocks binding of Dep-1 to a Sdc2 polypeptide. In certain embodiments, the anti-Sdc2 antibody inhibit Sdc2 activity. In some embodiments, the anti-Sdc2 antibody or antigen binding fragment thereof inhibits binding of Dep-1 to Sdc2.

In some embodiments, the antibody or antigen-binding fragment thereof result in reduced Dep-1 internalization. In some embodiments, the antibody or antigen-binding fragment thereof result in enhanced cell membrane level of Dep-1. In some embodiments, the antibody or antigen-binding fragment thereof result in enhance cell membrane level of Dep-1, without binding directly to the Dep-1 binding site of Sdc2. In some embodiments, the antibody or antigen-binding fragment thereof result in enhanced dephosphorylation of vascular endothelial growth factor receptor 2 (VEGFR2) protein by Dep-1. In specific embodiments, the antibody or antigen-binding fragment thereof result in enhanced dephosphorylation of VEGFR2 at residue Y951. In specific embodiments, the antibody or antigen-binding fragment thereof inhibits VEGFR2 signaling. In some embodiments, the anti-Sdc2 antibody binds Sdc2, leading to modulation of VEGFR2 or DEP1 signaling and/or molecular activities that result in inhibition of VEGFA-induced vascular permeability.

In some embodiments, the antibody or antigen-binding fragment thereof bind to the Sdc2 extracellular domain and promote stabilization of the endothelial cell junctions, thereby resulting in inhibition of vascular permeability. The invention is also based in part on the discovery that modulation of Sdc2 can be used to treat diseases whose pathogenesis is determined by vessel leakage or edema formation, including ischemic stroke among others.

In still other embodiments of the methods, the antibodies competitively block an anti-Sdc2 antibody provided herein from binding to a Sdc2 polypeptide.

In another embodiment of the methods, the antibodies compete for binding to a Sdc2 polypeptide with an anti-Sdc2 antibody provided herein. In some embodiments, the antibodies compete for binding to a Sdc2 polypeptide with a polyclonal anti-Sdc2 antibody Ab3 that binds to Sdc2 at the Dep-1 binding region in Sdc2.

The anti-Sdc2 antibodies provided herein can also be conjugated or recombinantly fused, e.g., to a diagnostic agent or detectable agent. Further provided are compositions comprising an anti-Sdc2 antibody.

In certain aspects, provided herein are methods of managing, preventing, or treating ischemic stroke in a subject, comprising administering to a subject an effective amount of binding proteins, such as antibodies that bind to Sdc2. Exemplary Sdc2 antibodies are provided herein. In one embodiment, provided herein is a method of managing ischemic stroke in a subject, comprising administering to a subject an effective amount of a Sdc2 binding protein. In one embodiment, provided herein is a method of preventing ischemic stroke in a subject, comprising administering to a subject an effective amount of a Sdc2 binding protein. In one embodiment, provided herein is a method of treating ischemic stroke in a subject, comprising administering to a subject an effective amount of a Sdc2 binding protein. In a specific embodiment, the Sdc2 binding protein is an antibody that binds to Sdc2. Exemplary Sdc2 antibodies useful in these methods are provided herein.

In certain embodiments, the Sdc2 binding proteins, including antibodies disclosed herein, are antagonist (e.g., inhibiting Sdc2 activity or signaling). In other embodiments, the Sdc2 antibodies inhibit Sdc2 activity. In some embodiments, the binding, competition, and/or signaling is assayed in vitro, e.g., in a cell-based assay. In other embodiments, the binding, competition, and/or signaling is assayed ex vivo, e.g., in an endothelial permeability assay. In other embodiments, the binding and/or signaling is assayed using a sample from a subject (e.g., a human subject). In other embodiments, the binding and/or signaling is assayed in vivo, e.g., in a mouse model or non-human primate of ischemic stroke (see, e.g., Example 9). In some embodiments, the activities are exhibited in vitro. In other embodiments, the activities are exhibited in vivo.

The discovery that such binding proteins, including anti-Sdc2 antibodies, inhibiting Sdc2 activity make them viable therapeutics for the treatment of ischemic stroke.

In specific embodiments of the various methods provided herein, the binding proteins, such as antibodies that bind to Sdc2, described herein share the common feature of competing with each other for the binding of Sdc2. This competitive inhibition can indicate that each antibody binds to the same region of Sdc2 (e.g., the same epitope), thereby asserting similar effects. In certain embodiments, anti-Sdc2 antibodies provided herein include humanized or fully human anti-Sdc2 antibodies, such as those derived from or based on antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, and/or R4M-P1-A10. In other embodiments, anti-Sdc2 antibodies provided herein compete for binding with an antibody derived from or based on 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, and/or R4M-P1-A10. In some embodiments, the anti-Sdc2 antibodies have CDR sequences as described in Tables 4-8. In certain embodiments, the anti-Sdc2 antibodies bind to the Dep-1 binding region of Sdc2 (e.g., residues 123-140 in the extracellular domain of human Sdc2; see Example 1). Taken together, the results described herein demonstrate that the effects observed for an anti-Sdc2 antibody that is derived from or based on 20-H19-AB, including an antibody having one or more CDRs described in Tables 4-8, can be extrapolated to other anti-Sdc2 antibodies provided herein having the same or similar epitope specificity (e.g., the same or similar CDRs). For example, the activities of antibodies as shown in Examples 4 and 5, for an exemplary humanized anti-Sdc2 antibody, are representative of the activities and effects of the anti-Sdc2 antibodies provided herein.

In some embodiments of the various methods provided herein, the binding proteins such as anti-Sdc2 antibodies may comprise immunoglobulin variable regions which comprise one or more CDRs as described in Tables 4-8. In such binding proteins (e.g., anti-Sdc2 antibodies), the CDRs may be joined with one or more scaffold regions or framework regions (FRs), which orient(s) the CDR(s) such that the proper antigen binding properties of the CDR(s) is achieved.

In certain embodiments, the method comprises administering a Sdc2 binding protein provided herein to the subject in an amount effective for treating the ischemic stroke. Also provided herein are methods of managing ischemic stroke in a subject. In certain embodiments, the method comprises administering a Sdc2 binding protein provided herein to the subject in an amount effective for managing the ischemic stroke. Also provided herein are methods of preventing ischemic stroke in a subject. In certain embodiments, the method comprises administering a Sdc2 binding protein provided herein to the subject in an amount effective for preventing the ischemic stroke. In some embodiments, the subject has ischemic stroke. In some embodiments, the subject has dry ischemic stroke. In some embodiments, the subject has wet ischemic stroke. In other embodiments, the subject is at risk of having ischemic stroke. In other embodiments, the subject is at risk of having dry ischemic stroke. In other embodiments, the subject is at risk of having wet ischemic stroke. In one embodiment, the subject is a subject in need thereof. In a specific embodiment, the Sdc2 binding protein is a Sdc2 antibody provided herein. In certain embodiments, the Sdc2 binding protein is an antigen binding fragment of a Sdc2 antibody provided herein.

In other embodiments, the anti-Sdc2 antibodies are fully human or humanized antibodies (e.g., comprising human constant regions) that bind Sdc2, including a Sdc2 polypeptide, a Sdc2 polypeptide fragment, a Sdc2 peptide, or a Sdc2 epitope. In certain embodiments, the anti-Sdc2 antibody comprises a VH region, VL region, VH CDR1, VH CDR2, VH CDR3, VL CDR1, VL CDR2, and/or VL CDR3 of any one of the monoclonal antibodies provided herein, such as an amino acid sequence depicted in Tables 1 and 3-8. Accordingly, in some embodiments, the isolated antibody or functional fragment thereof provided herein comprises one, two, and/or three heavy chain CDRs and/or one, two, and/or three light chain CDRs from: (a) the antibody 20-H19-AB, (b) the antibody TP-43327F, (c) the antibody TP-43329F, (d) the antibody 8-G17-A, (e) the antibody 6-N03-A, (f) the antibody R3-P3-C11, (g) the antibody R4M-P3-E06, (h) the antibody R3-P3-E09, (i) the antibody R3-P1-C02, (j) the antibody R3-P3-A12, (k) the antibody R4M-P3-A12, and (l) the antibody R4M-P1-A10, as shown in Tables 1 and 3-8.

In some embodiments, provided herein are methods for preventing, managing or treating ischemic stroke is a subject in need thereof, the method comprising administering to the subject a therapeutic effective amount of an antibody that binds to Sdc2 or an antigen binding fragment thereof. In some embodiments, the anti-Sdc2 antibody or an antigen binding fragment thereof is administered systemically to the subject. In some embodiments, the anti-Sdc2 antibody or an antigen binding fragment is administered intravenously to the subject. In some embodiments, the anti-Sdc2 antibody or an antigen binding fragment is administered intraocularly into the eye of the subject. In some embodiments, the anti-Sdc2 antibody or an antigen binding fragment is administered intravitreally into the eye of the subject. In some embodiments, the Sdc2 antibody is administered to the subject via gene therapy.

Any of the Sdc2 antibodies provided herein are contemplated for use in the methods provided herein. In one embodiment of the methods provided herein, the Sdc2 antibody is 20-H19-AB. In one embodiment, the Sdc2 antibody is derived from 20-H19-AB. In one embodiment, the Sdc2 antibody is an antibody fragment of 20-H19-AB. In one embodiment, the Sdc2 antibody comprises a VH amino acid sequence of 20-H19-AB. In one embodiment, the Sdc2 antibody comprises a VL amino acid sequence of 20-H19-AB. In one embodiment, the Sdc2 antibody comprises a VH amino acid sequence and VL amino acid sequence of 20-H19-AB. In one embodiment, the Sdc2 antibody comprises a VH CDR1, VH CDR2 and VH CDR3 amino acid sequence of 20-H19-AB. In one embodiment, the Sdc2 antibody comprises a VL CDR1, VL CDR2 and VL CDR3 amino acid sequence of 20-H19-AB. In one embodiment, the Sdc2 antibody comprises a VH CDR1, VH CDR2, VH CDR3, VL CDR1, VLCDR2 and VL CDR3 amino acid sequence of 20-H19-AB. In some embodiments, the antibody is an IgG antibody. In some embodiments, the antibody is an IgG1 antibody. In a specific embodiment, the antibody is an IgG4 antibody. Other suitable Sdc2 antibodies are provided in Section 6.7.4 below and elsewhere herein.

In certain embodiments of the methods provided herein, upon administering the antibody that binds to Sdc2, the endothelial cell permeability in or surrounding the brain lesion area in the subject is reduced. In specific embodiments, upon administering the antibody that binds to Sdc2, the endothelial cell permeability is reduced at least about 10%, at least about 20%, at least about 30%, at least about 40%, at least about 50%, at least about 60%, at least about 70%, at least about 80%, at least about 90%, or at least about 95%. In some embodiments, the reduction in endothelial cell permeability is detected using a Miles assay.

In certain embodiments of the methods provided herein, upon administering the antibody that binds to Sdc2, the vascular permeability in or surrounding the brain lesion area in the subject is reduced. In specific embodiments, upon administering the antibody that binds to Sdc2, the vascular permeability is reduced at least about 10%, at least about 20%, at least about 30%, at least about 40%, at least about 50%, at least about 60%, at least about 70%, at least about 80%, at least about 90%, or at least about 95%. In some embodiments, the endothelial cell permeability is VEGFA-induced endothelial cell permeability. In certain embodiments, at least some of the endothelial cells expresses Sdc2. In some embodiments, the reduction in vascular permeability is detected using a Miles assay.

In certain embodiments, the brain lesion area comprises a penumbra. In some embodiments, upon administering the antibody that binds to Sdc2, the penumbra is reduced. In specific embodiments, upon administering the antibody that binds to Sdc2, the penumbra is reduced at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100% in size. In some embodiments, the reduction in size is measured in volume or area. In some embodiments, the reduction is size is detected by MRI.

In certain embodiments, the brain lesion area comprises an edema. In some embodiments, upon administering the antibody that binds to Sdc2, the edema is reduced. In specific embodiments, upon administering the antibody that binds to Sdc2, the edema is reduced at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100% in size. In some embodiments, the reduction in size is measured in volume or area. In some embodiments, the reduction is size is detected by MRI.

In certain embodiments, the brain lesion area comprises an infarct. In some embodiments, upon administering the antibody that binds to Sdc2, the infarct is reduced. In specific embodiments, upon administering the antibody that binds to Sdc2, the infarct is reduced at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100% in size. In some embodiments, the reduction in size is measured in volume or area. In some embodiments, the reduction is size is detected by MRI.

In certain embodiments of the methods provided herein, the subject is a human suffering or at risk of suffering from an ischemic stroke.

### 6.7.2 Methods of Managing, Preventing or Treating Age-Related Macular Degeneration and Other Neovascular Eye Diseases

In one embodiment, provided is a method of treating a neovascular eye disease in a subject, comprising administering to the subject an effective amount of a Sdc2 antibody provided herein. In one embodiment, provided is a method of treating diabetic retinopathy in a subject, comprising administering to the subject an effective amount of a Sdc2 antibody provided herein. In one embodiment, provided is a method of treating AMD in a subject, comprising administering to the subject an effective amount of a Sdc2 antibody provided herein. In a specific embodiment, the AMD is wet AMD. In certain embodiments, the antibody is administered directly to the eye of the subject. In some embodiments, the antibody is intraocularly administered. In certain embodiments, the antibody is intravitreally administered.

In one aspect, provided are methods of managing, preventing, or treating AMD in a subject, comprising administering to a subject a therapeutically effective amount of an antibody that binds to a Sdc2 polypeptide, a Sdc2 polypeptide fragment, a Sdc2 peptide, or a Sdc2 epitope.

In certain embodiments of the methods, the antibodies provided herein bind to human and/or cyno Sdc2. In one embodiment, the Sdc2 antibodies bind to human Sdc2. In one embodiment, the Sdc2 antibodies bind to cyno Sdc2. In other embodiments, the antibodies bind to rodent Sdc2 (e.g., a mouse Sdc2). In other embodiments, the antibodies bind to porcine Sdc2 (e.g., a pig Sdc2). In another embodiment, the antibody binds to human Sdc2 and cyno Sdc2. In some embodiments, the antibody binds to a rodent Sdc2 (e.g., a mouse Sdc2). In some embodiments, the antibody binds to a porcine Sdc2 (e.g., a pig Sdc2). In certain embodiments, the antibodies bind to human and/or cyno Sdc2 but does not bind to rodent Sdc2. In some embodiments, the antibody or antigen-binding fragment binds specifically to an epitope which is shared by human, mouse, and porcine Sdc2 proteins or any combination thereof (i.e., human and mouse, mouse and porcine, or porcine and human).

In some embodiments, the anti-Sdc2 antibodies inhibits the binding of Dep-1 to a Sdc2 polypeptide. In some embodiments, the anti-Sdc2 antibodies competes with Dep-1 for binding to a Sdc2 polypeptide. In some embodiments, the anti-Sdc2 antibody or antigen binding fragment thereof binds to a region in the extracellular domain of Sdc2 that correspond to amino acids 123 to 140 of human Sdc2 (SEQ ID NO:25).

In some embodiments of the methods, the anti-Sdc2 antibodies bind to the extracellular domain (ECD) of Sdc2. In certain embodiments, the anti-Sdc2 antibodies bind to an epitope in the ECD of Sdc2. In certain embodiments, the anti-Sdc2 antibodies bind to an epitope in the ECD of Sdc2 to which Dep-1 binds. In some embodiments, the anti-Sdc2 antibodies compete with Dep-1 for binding to a Sdc2 polypeptide. In some embodiments, the anti-Sdc2 antibodies inhibits binding of Dep-1 to a Sdc2 polypeptide. In some embodiments, the anti-Sdc2 antibody blocks binding of Dep-1 to a Sdc2 polypeptide. In certain embodiments, the anti-Sdc2 antibody inhibit Sdc2 activity. In some embodiments, the anti-Sdc2 antibody or antigen binding fragment thereof inhibits binding of Dep-1 to Sdc2.

In some embodiments, the antibody or antigen-binding fragment thereof result in reduced Dep-1 internalization. In some embodiments, the antibody or antigen-binding fragment thereof result in enhanced cell membrane level of Dep-1. In some embodiments, the antibody or antigen-binding fragment thereof result in enhance cell membrane level of Dep-1, without binding directly to the Dep-1 binding site of Sdc2. In some embodiments, the antibody or antigen-binding fragment thereof result in enhanced dephosphorylation of vascular endothelial growth factor receptor 2 (VEGFR2) protein by Dep-1. In specific embodiments, the antibody or antigen-binding fragment thereof result in enhanced dephosphorylation of VEGFR2 at residue Y951. In specific embodiments, the antibody or antigen-binding fragment thereof inhibits VEGFR2 signaling. In some embodiments, the anti-Sdc2 antibody binds Sdc2, leading to modulation of VEGFR2 or DEP1 signaling and/or molecular activities that result in inhibition of VEGFA-induced vascular permeability.

In some embodiments, the antibody or antigen-binding fragment thereof bind to the Sdc2 extracellular domain and promote stabilization of the endothelial cell junctions, thereby resulting in inhibition of vascular permeability. The invention is also based in part on the discovery that modulation of Sdc2 can be used to treat diseases whose pathogenesis is determined by vessel leakage or edema formation, including AMD among others.

In still other embodiments of the methods, the antibodies competitively block an anti-Sdc2 antibody provided herein from binding to a Sdc2 polypeptide.

In another embodiment of the methods, the antibodies compete for binding to a Sdc2 polypeptide with an anti-Sdc2 antibody provided herein. In some embodiments, the antibodies compete for binding to a Sdc2 polypeptide with a polyclonal anti-Sdc2 antibody Ab3 that binds to Sdc2 at the Dep-1 binding region in Sdc2.

The anti-Sdc2 antibodies provided herein can also be conjugated or recombinantly fused, e.g., to a diagnostic agent or detectable agent. Further provided are compositions comprising an anti-Sdc2 antibody.

In certain aspects, provided herein are methods of managing, preventing, or treating AMD in a subject, comprising administering to a subject an effective amount of binding proteins, such as antibodies that bind to Sdc2. Exemplary Sdc2 antibodies are provided herein. In one embodiment, provided herein is a method of managing AMD in a subject, comprising administering to a subject an effective amount of a Sdc2 binding protein. In one embodiment, provided herein is a method of preventing AMD in a subject, comprising administering to a subject an effective amount of a Sdc2 binding protein. In one embodiment, provided herein is a method of treating AMD in a subject, comprising administering to a subject an effective amount of a Sdc2 binding protein. In a specific embodiment, the Sdc2 binding protein is an antibody that binds to Sdc2. Exemplary Sdc2 antibodies useful in these methods are provided herein.

In certain embodiments, the Sdc2 binding proteins, including antibodies disclosed herein, are antagonist (e.g., inhibiting Sdc2 activity or signaling). In other embodiments, the Sdc2 antibodies inhibit Sdc2 activity. In some embodiments, the binding, competition, and/or signaling is assayed in vitro, e.g., in a cell-based assay. In other embodiments, the binding, competition, and/or signaling is assayed ex vivo, e.g., in an endothelial permeability assay. In other embodiments, the binding and/or signaling is assayed using a sample from a subject (e.g., a human subject). In other embodiments, the binding and/or signaling is assayed in vivo, e.g., in a mouse model of AMD (see, e.g., Example 8). In some embodiments, the activities are exhibited in vitro. In other embodiments, the activities are exhibited in vivo.

The discovery that such binding proteins, including anti-Sdc2 antibodies, inhibiting Sdc2 activity make them viable therapeutics for the treatment of AMD.

In specific embodiments of the various methods provided herein, the binding proteins, such as antibodies that bind to Sdc2, described herein share the common feature of competing with each other for the binding of Sdc2. This competitive inhibition can indicate that each antibody binds to the same region of Sdc2 (e.g., the same epitope), thereby asserting similar effects. In certain embodiments, anti-Sdc2 antibodies provided herein include humanized or fully human anti-Sdc2 antibodies, such as those derived from or based on antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, and/or R4M-P1-A10. In other embodiments, anti-Sdc2 antibodies provided herein compete for binding with an antibody derived from or based on 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, and/or R4M-P1-A10. In some embodiments, the anti-Sdc2 antibodies have CDR sequences as described in Tables 4-8. In certain embodiments, the anti-Sdc2 antibodies bind to the Dep-1 binding region of Sdc2 (e.g., residues 123-140 in the extracellular domain of human Sdc2; see Example 1). Taken together, the results described herein demonstrate that the effects observed for an anti-Sdc2 antibody that is derived from or based on 20-H19-AB, including an antibody having one or more CDRs described in Tables 4-8, can be extrapolated to other anti-Sdc2 antibodies provided herein having the same or similar epitope specificity (e.g., the same or similar CDRs). For example, the activities of antibodies as shown in Examples 4 and 5, for an exemplary humanized anti-Sdc2 antibody, are representative of the activities and effects of the anti-Sdc2 antibodies provided herein.

In some embodiments of the various methods provided herein, the binding proteins such as anti-Sdc2 antibodies may comprise immunoglobulin variable regions which comprise one or more CDRs as described in Tables 4-8. In such binding proteins (e.g., anti-Sdc2 antibodies), the CDRs may be joined with one or more scaffold regions or framework regions (FRs), which orient(s) the CDR(s) such that the proper antigen binding properties of the CDR(s) is achieved.

In certain embodiments, the method comprises administering a Sdc2 binding protein provided herein to the subject in an amount effective for treating the AMD. Also provided herein are methods of managing AMD in a subject. In certain embodiments, the method comprises administering a Sdc2 binding protein provided herein to the subject in an amount effective for managing the AMD. Also provided herein are methods of preventing AMD in a subject. In certain embodiments, the method comprises administering a Sdc2 binding protein provided herein to the subject in an amount effective for preventing the AMD. In some embodiments, the subject has AMD. In some embodiments, the subject has dry AMD. In some embodiments, the subject has wet AMD. In other embodiments, the subject is at risk of having AMD. In other embodiments, the subject is at risk of having dry AMD. In other embodiments, the subject is at risk of having wet AMD. In one embodiment, the subject is a subject in need thereof. In a specific embodiment, the Sdc2 binding protein is a Sdc2 antibody provided herein. In certain embodiments, the Sdc2 binding protein is an antigen binding fragment of a Sdc2 antibody provided herein.

In other embodiments, the anti-Sdc2 antibodies are fully human or humanized antibodies (e.g., comprising human constant regions) that bind Sdc2, including a Sdc2 polypeptide, a Sdc2 polypeptide fragment, a Sdc2 peptide, or a Sdc2 epitope. In certain embodiments, the anti-Sdc2 antibody comprises a VH region, VL region, VH CDR1, VH CDR2, VH CDR3, VL CDR1, VL CDR2, and/or VL CDR3 of any one of the monoclonal antibodies provided herein, such as an amino acid sequence depicted in Tables 1 and 3-8. Accordingly, in some embodiments, the isolated antibody or functional fragment thereof provided herein comprises one, two, and/or three heavy chain CDRs and/or one, two, and/or three light chain CDRs from: (a) the antibody 20-H19-AB, (b) the antibody TP-43327F, (c) the antibody TP-43329F, (d) the antibody 8-G17-A, (e) the antibody 6-N03-A, (f) the antibody R3-P3-C11, (g) the antibody R4M-P3-E06, (h) the antibody R3-P3-E09, (i) the antibody R3-P1-C02, (j) the antibody R3-P3-A12, (k) the antibody R4M-P3-A12, and (l) the antibody R4M-P1-A10, as shown in Tables 1 and 3-8.

In some embodiments, provided herein are methods for preventing, managing or treating AMD is a subject in need thereof, the method comprising administering to the subject a therapeutic effective amount of an antibody that binds to Sdc2 or an antigen binding fragment thereof. In some embodiments, the anti-Sdc2 antibody or an antigen binding fragment thereof is administered systemically to the subject. In some embodiments, the anti-Sdc2 antibody or an antigen binding fragment is administered intravenously to the subject. In some embodiments, the anti-Sdc2 antibody or an antigen binding fragment is administered intraocularly into the eye of the subject. In some embodiments, the anti-Sdc2 antibody or an antigen binding fragment is administered intravitreally into the eye of the subject. In some embodiments, the Sdc2 antibody is administered to the subject via gene therapy.

Any of the Sdc2 antibodies provided herein are contemplated for use in the methods provided herein. In one embodiment of the methods provided herein, the Sdc2 antibody is 20-H19-AB. In one embodiment, the Sdc2 antibody is derived from 20-H19-AB. In one embodiment, the Sdc2 antibody is an antibody fragment of 20-H19-AB. In one embodiment, the Sdc2 antibody comprises a VH amino acid sequence of 20-H19-AB. In one embodiment, the Sdc2 antibody comprises a VL amino acid sequence of 20-H19-AB. In one embodiment, the Sdc2 antibody comprises a VH amino acid sequence and VL amino acid sequence of 20-H19-AB. In one embodiment, the Sdc2 antibody comprises a VH CDR1, VH CDR2 and VH CDR3 amino acid sequence of 20-H19-AB. In one embodiment, the Sdc2 antibody comprises a VL CDR1, VL CDR2 and VL CDR3 amino acid sequence of 20-H19-AB. In one embodiment, the Sdc2 antibody comprises a VH CDR1, VH CDR2, VH CDR3, VL CDR1, VLCDR2 and VL CDR3 amino acid sequence of 20-H19-AB. In some embodiments, the antibody is an IgG antibody. In some embodiments, the antibody is an IgG1 antibody. In a specific embodiment, the antibody is an IgG4 antibody. Other suitable Sdc2 antibodies are provided in Section 6.7.4 below and elsewhere herein.

In some embodiments, upon administration of the anti-Sdc2 antibody or antigen binding fragment thereof, endothelial permeability in the eye tissue of the subject is reduced. In some embodiments, upon administration of the anti-Sdc2 antibody or antigen binding fragment thereof to the subject, endothelia permeability in the eye tissue is reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100%. In some embodiments, the eye tissue is in the fundus of the eye (eye fundus tissue). In some embodiments, the eye tissue comprises the retina of the eye. In some embodiments, the eye tissue comprises the macula of the eye. In some embodiments, the eye tissue comprises the choroid of the eye. In some embodiments, the endothelial permeability is VEGFA-induced endothelial permeability. In some embodiments, the endothelial permeability is measured by fundus fluorescein angiography (FFA). In some embodiments, at least some of the endothelial cells expresses Sdc2.

In some embodiments, upon administering of the antibody or antigen binding fragment thereof, Dep-1 expression on the surface of cells in an eye fundus tissue is upregulated. In some embodiments, upon administering of the antibody or antigen binding fragment thereof, dephosphorylation of vascular endothelial growth factor receptor 2 (VEGFR2) protein at residue Y951 is enhanced in cells in an eye fundus tissue. In some embodiments, upon administering of the antibody or antigen binding fragment thereof, inflammation in an eye fundus tissue is reduced. In some embodiments, expression of one or more inflammatory marker in the eye fundus tissue is reduced. In one embodiment, the inflammatory marker is a pro-inflammatory cytokine or an immune cell surface protein. In one embodiment, the inflammatory marker is F4/80. In some embodiments, upon administering of the antibody or antigen binding fragment thereof, does not impact angiogenesis in an eye fundus tissue. In some embodiments, the angiogenesis is choroidal neovascularization (CNV). In some embodiments, expression of one or more endothelial marker remains the same in the eye fundus.

In some embodiments, upon administration of the anti-Sdc2 antibody or antigen binding fragment thereof, vascular permeability in the eye tissue of the subject is reduced. In some embodiments, upon administration of the anti-Sdc2 antibody or antigen binding fragment thereof to the subject, vascular permeability in the eye tissue is reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100%. In some embodiments, the eye tissue is in the fundus of the eye. In some embodiments, the eye tissue comprises the retina of the eye. In some embodiments, the eye tissue comprises the macula of the eye. In some embodiments, the eye tissue comprises the choroid of the eye. In some embodiments, the eye tissue is in the fundus of the eye. In some embodiments, the eye tissue comprises the retina of the eye. In some embodiments, the eye tissue comprises the macula of the eye. In some embodiments, the eye tissue comprises the choroid of the eye. In some embodiments, the vascular permeability is measured by fundus fluorescin angiography (FFA).

In some embodiments, upon administration of the anti-Sdc2 antibody or antigen binding fragment thereof, the central retinal thickness in the eye of the subject is reduced. In some embodiments, upon administration of the anti-Sdc2 antibody or antigen binding fragment thereof, the central retinal thickness is reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100%. In some embodiments, the central retinal thickness is measured by optical coherence tomography (OCT).

In some embodiments, expression of one or more endothelial marker remains the same in the eye fundus tissue. In one embodiment, the endothelial marker is an ETS-related gene (ERG). In some embodiments, the eye fundus tissue is the retina, macula or choroid of the eye.

In some embodiments, upon administration of the anti-Sdc2 antibody or antigen binding fragment thereof, does not impact angiogenesis in the eye tissue of the subject. In some embodiments, the eye tissue comprises the choroid of the eye. In some embodiments, the reduced angiogenesis is choroidal neovascularization (CNV).

In some embodiments, upon administration of the anti-Sdc2 antibody or antigen binding fragment thereof, expression of one or more endothelial marker protein in an eye tissue remains substantially the same in the subject. In some embodiments, the eye tissue is in the fundus of the eye. In some embodiments, the eye tissue comprises the retina of the eye. In some embodiments, the eye tissue comprises the macula of the eye. In some embodiments, the eye tissue comprises the choroid of the eye. In some embodiments, the one or more endothelial marker protein is selected from are selected from CD31 (PECAM1), VWF, CD93, EGFL7, ID3, FLT1, GNG11, MCAM, FLT4, PLVAP, ADGRF5, ABCG2, ACVRL1, NRP2, FHL2, ARHGEF15, EMCN, ADGRL4, PTPRB, CLDN5, ELK3, CDH5, ENG, KDR, EPAS1, ETS1, CD34, CLIC4, LY6A, IGFBP7, ID1, ICAM1, ITGB3, SELE, VCAM1, PROCR, TEK, APLN, NOS3, THBD, ACKR1, SLCO1C1, TMEM100, ABCB1A, PODXL, NOSTRIN, MFSD2A, AQP1, MYLK, RASIP1, FLI1, TIE1, APLNR, ADAMTS1, RPRM, FABP4, LOX, CARD10, CLEC14A, DLL4, ESM1, EXOC3L, GIMAP5, GJA4, MMRN2, NOTCH4, NPR1, PRKCH, RASGRP3, ROBO4, SCARF1, SOX18, SOX7, SPNS2, THSD1, APOLD1, EMP1, CD36, RNASE1, CTGF, HYAL2, CLEC4G, GPR182, F8, RBP7, CALCRL, FOXF1, CASZ1, AQP7, TCF15, CD300LG, BTNL9, MEOX2, ERG, HEXIM1, GLYCAM1, CD55, MMRN1, C7, RAMP3, VEGFC, GJA5, HEY1, RND1, BDP1, CD46, MEOX1, CCL19, CLCA3A1, MADCAM1, CYP1B1, IRX3, BIRC2, LYVE1, SEMA3D, SLCO1A4, WFDC1, VWA1, ECE1, SDPR, CAR4, TBX1, SEMA7A, FOXF2, PDGFB, ECSCR, PLEC, STAB1, TGFBR2, CXCL1, RGS5, SLC7A5, SLC2A1, EDNRB, KCNJ8, CD82, CHST1, PLAC8, TSPAN8, PDPN, PROX1, EHD3, SRGN, S100A10, USHBP1, MYF6, OIT3, IL1A, BMP2, C1QTNF1, PCDH12, DPP4, PALMD, POSTN, BMX, SLC38A5, XDH, SPARC, MGLL, SLC9A3R2, RGCC, ICAM2, MGP, SPARCL1, TM4SF1, ADIRF, CD9, SRPX, CAV1, HSPG2, CCL14, CLEC1B, FCN2, S100A13, FCN3, CRHBP, IFI27, CCL23, SGK1, DNASE1L3, LIFR, PCAT19, CDKN1C, INMT, PTGDS, TIMP3, GPM6A, FAM167B, LTC4S, STAB2, GPIHBP1, KLK1, ADORA2A, ARAP3, and LY6C1. In particular embodiments, upon administration of the anti-Sdc2 antibody or antigen binding fragment thereof, expression of CD31 remains substantially the same in the eye tissue. In particular embodiments, upon administration of the anti-Sdc2 antibody or antigen binding fragment thereof, expression of ERG remains substantially the same in the eye tissue. In some embodiments, the eye tissue is in the fundus of the eye. In some embodiments, the eye tissue comprises the retina of the eye. In some embodiments, the eye tissue comprises the macula of the eye. In some embodiments, the eye tissue comprises the choroid of the eye.

In one aspect, provided here is also a method for reducing eye inflammation in a subject in need thereof. In some embodiments, the method comprises administering to the subject a therapeutic effective amount of an antibody that binds Sdc2 or an antigen binding fragment thereof, wherein upon administering of the antibody or antigen binding fragment thereof, one or more inflammation marker is reduced in the choroid of an eye of the subject. In some embodiments, the inflammation marker is CD31 or F4/80. In some embodiments, upon administering of the antibody or antigen binding fragment thereof, expression of one or more endothelial marker in the choroid of the eye remains substantially the same. In some embodiments, the inflammatory marker is ERG.

In some embodiments, upon administration of the anti-Sdc2 antibody or antigen binding fragment thereof, inflammation in an eye tissue of the subject is reduced. In some embodiments, upon administration of the anti-Sdc2 antibody or antigen binding fragment thereof to the subject, expression of one or more pro-inflammatory cytokines in the eye tissue is reduced. In some embodiments, expression of one or more pro-inflammatory cytokines in the eye tissue is reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100%. In some embodiments, the cytokine is IL-1, IL-2, IL-6, IL-12, IL-17, IL-22, IL-23, GM-CSF, TNF-α, IFN-γ, or a combination thereof. In some embodiments, the eye tissue is in the fundus of the eye. In some embodiments, the eye tissue comprises the retina of the eye. In some embodiments, the eye tissue comprises the macula of the eye. In some embodiments, the eye tissue comprises the choroid of the eye.

In some embodiments, upon administration of the anti-Sdc2 antibody or antigen binding fragment thereof to the subject, recruitment of immune cells to the eye tissue is reduced in the subject. In some embodiments, recruitment of immune cells to the eye tissue is reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100%. In some embodiments, the immune cell is selected from T cells, B cells, natural killer cells, neutrophils, mast cells, macrophages, antigen-presenting cells (APC), basophils, and eosinophils. In some embodiments, the eye tissue is in the fundus of the eye. In some embodiments, the eye tissue comprises the retina of the eye. In some embodiments, the eye tissue comprises the macula of the eye. In some embodiments, the eye tissue comprises the choroid of the eye.

In some embodiments, upon administration of the anti-Sdc2 antibody or antigen binding fragment thereof, expression of one or more T cell markers in the eye tissue is reduced. In some embodiments, expression of one or more T cell markers in the eye tissue is reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100%. In some embodiments, the T cell marker is selected from TRBC2, CD3D, CD3G, CD3E, LTB, IL7R, LEF1, GZMK, TRAC, HOPX, IFIT3, CERK, GIMAP3, CXCR6, SATB1, PTPRCAP, CD69, TRBC1, CD2, GDPD3, IL2RA, CCL5, MYB, GZMA, CD52, CCL4, GIMAP2, SYT3, NOTCH3, SEMA6D, DKK3, PMCH, ITK, GEM, MAFF, TGIF1, RORA, TNFAIP3, CREM, PXDC1, NABP1, FAM110A, EEF1B2P3, PFN1P1, IL32, CXCR4, SEPT1, BCL2, CYTL1, CTSW, PTPN22, TXK, TRAF1, CD8B, BATF3, GZMH, LAG3, CD8B1, GZMB, SH2D1A, MYO1G, FMNL1, S1PR4, CD247, MS4A4C, GIMAP5, CD28, CD160, TCRG-C2, TCRG-C1, TCRG-C4, TRDC, H2-T3, H2-T10, RHOH, KLRB1, CCR2, IL2RB, CD163L1, TRDV4, MBD2, ICOS, IL18R1, TNFRSF4, CCL20, CLEC2D, CD8A, CD6, S100A4, LCK, CD81, THY1, LAT, SKAP1, TCF7, CCL4L2, PYHIN1, JUNB, DUSP2, IFNG, BRAF, CCL3, CCL6, CD4, CD7, H2-Q7, and CCR7.

In some embodiments, upon administration of the anti-Sdc2 antibody or antigen binding fragment thereof, expression of one or more B cell markers in the eye tissue is reduced. In some embodiments, expression of one or more B cell markers in the eye tissue is reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100%. In some embodiments, the B cell marker is selected from PXK, CD19, MS4A1, CD74, CD79A, PTPRC, IGHD, IGHM, HLA-DRA, PAX5, BANK1, CR2, CD22, FCER2, CD79B, IGLL1, SPN, B3GAT1, CD72, LY6D, IGLC1, CD5, CD40, CD69, CD70, CD86, TNFRSF9, SDC1, TNFSF4, TNFRSF13B, TNFRSF13C, PDCD1, RASGRP3, HLA-DQA1, FLI1, CD14, SEMA6D, LAIR1, IFIT3, DNTT, CD24, CD27, MUM1, JCHAIN, MZB1, H2-DMB2, FCMR, EDEM1, VPREB3, H2-OB, POU2AF1, CRELD2, DERL3, SIGLECG, RALGPS2, FCHSD2, POLD4, CMAH, TNFRSF17, HVCN1, TRP53INP1, FCRLA, EDEM2, BLNK, H2-OA, TXNDC11, BTLA, SMAP2, SCD1, FAM46C, FKBP11, SEC61A1, SPCS3, SPIB, EAF2, CXCR4, BIRC3, IGLC2, IGLC3, IL21R, IGKC, IGLV1, VPREB1, VPREB2, LRMP, KLHL6, SLAMF6, FAM129C, BST1, MSH5, DOK3, BACH2, FCER2A, IGHG3, IGHG4, IGHA, CD38, EBF1, BCL11A, CCR7, CD55, CD52, TLR9, SWAP70, HMGA1, CD2, CD80, LTB, MME, and IGHG1.

In some embodiments, upon administration of the anti-Sdc2 antibody or antigen binding fragment thereof, expression of one or more natural killer cell markers in the eye tissue is reduced. In some embodiments, expression of one or more natural killer cell markers in the eye tissue is reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100%. In some embodiments, the natural killer cell marker is selected from NCAM1, IL2RB, CD44, IL12RB2, CXCR4, ZFP683, SLAMF7, TCF7, STYK1, S1PR1, GATA3, TBX21, ZBTB16, PRF1, TNFRSF8, GZMB, NR4A1, MAP3K8, EGR1, ITGB2, RORA, KLRB1, IL12RB1, and IL17RA.

In some embodiments, upon administration of the anti-Sdc2 antibody or antigen binding fragment thereof, expression of one or more macrophage markers in the eye tissue is reduced. In some embodiments, expression of one or more macrophage markers in the eye tissue is reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100%. In some embodiments, the macrophage marker is selected from CD68, FCGR1, NAAA, JAML, TYROBP, TREM2, H2-DMA, CXCL16, MGL2, CLEC4A2, CCL12, PARP14, SEPP1, MARCH1, FGL2, LYZ2, WFDC17, CPM, CHIT1, TGFBR1, SLAMF9, SCIMP, LILRA5, C3AR1, FGD2, RAB7B, RBPJ, SLCO2B1, EGLN3, CLEC4D, ADAM8, ARL11, MMP12, VSIG4, RETNLA, RAB20, SNX20, FMNL1, GPR132, NCEH1, CCL24, CD300A, CCL7, IRF5, MYO1G, DUSP5, GPR171, SAMSN1, NR4A3, HILPDA, SLC37A2, LPCAT2, IFNAR2, MS4A7, HPGD, CD5L, LILR4B, CYTH4, CLEC4N, CD209F, CSF1R, FGR, CYBB, CD200, CD200R1, GATA6, H2-AB1, H2-EB1, MAF, AIF1, ADGRE5, CLEC4A3, CLEC4A1, SLC15A3, AKR1B3, CYP27A1, ITGAL, ITGAM, CD14, FUT4, FCGR3A, CD33, FCGR1A, CD80, LILRB4, CD86, CD163, CCR5, TLR2, TLR4, ADGRE1 (f4/80), GPR34, CCL9, FABP4, S100A8, F13A1, CD83, STAB1, MRC1, CCL6, GPNMB, LYVE1, PLTP, MS4A4A, MS4A6A, FPR1, CD180, GDF15, HFE, TNF, CCR2, CD209A, C5AR1, CXCL2, CCL2, IL1B, AHR, CCR7, DNASE1L3, CXCL1, CCL22, S100A4, MMP9, NRP2, CTSK, CD36, HPGDS, SLC11A1, CCL3, CLEC7A, CCL5, CD3E, CD19, CD74, LYZ1, LGALS3, UCP2, TREML4, IL4RA, ITGAX, LY6C1, PPARG, SIGLECF, RGS1, DAB2, P2RY6, CLEC10A, ADGRE4, RUNX3, SYK, CX3CR1, and SLAMF7, and CD31 (PECAM1).

In specific embodiments, upon administration of the anti-Sdc2 antibody or antigen binding fragment thereof, ADGRE1 expression level in the eye tissue is reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100%. In specific embodiments, upon administration of the anti-Sdc2 antibody or antigen binding fragment thereof, CD31 expression level in the eye tissue is reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100%.

In some embodiments, upon administration of the anti-Sdc2 antibody or antigen binding fragment thereof to the subject, vision acuity of the subject is enhanced.

In some embodiments, the subject is a human suffering from or at risk of developing AMD.

Moreover, an anti-Sdc2 antibody or fragment thereof can be administered to a non-human mammal expressing Sdc2 with which the antibody cross-reacts (e.g., a primate) for veterinary purposes or as an animal model of human disease. Regarding the latter, such animal models may be useful for evaluating the therapeutic efficacy of antibodies of the present disclosure (e.g., testing of dosages and time courses of administration).

In some embodiments of the various methods provided herein, the method further comprises administering a therapeutically effective amount of a second active agent or a support care therapy. Second active agents can be large molecules (e.g., proteins) or small molecules (e.g., synthetic inorganic, organometallic, or organic molecules). In some embodiments, the second active agents are small molecules that can alleviate adverse effects associated with the administration of an antibody provided herein. However, like some large molecules, many are believed to be capable of providing a synergistic effect when administered with (e.g., before, after or simultaneously) an antibody provided herein. Examples of small molecule second active agents include, but are not limited to, anti-cancer agents, anti-inflammatory agents, immunosuppressive agents, and steroids.

### 6.7.3 Methods of Managing, Preventing or Treating Myocardial Infarction and Other Cardiovascular Diseases

In one embodiment, provided is a method of treating a cardiovascular disease in a subject, comprising administering to the subject an effective amount of a Sdc2 antibody provided herein. In one embodiment, provided is a method of treating a myocardial infarction in a subject, comprising administering to the subject an effective amount of a Sdc2 antibody provided herein. In one embodiment, provided is a method of treating congestive heart failure in a subject, comprising administering to the subject an effective amount of a Sdc2 antibody provided herein.

Provided herein are methods of managing, preventing, or treating AMI in a subject, comprising administering to a subject a therapeutically effective amount of an antibody that binds to a Sdc2 polypeptide, a Sdc2 polypeptide fragment, a Sdc2 peptide, or a Sdc2 epitope.

In certain embodiments of the methods, the antibodies provided herein bind to human and/or cyno Sdc2. In one embodiment, the Sdc2 antibodies bind to human Sdc2. In one embodiment, the Sdc2 antibodies bind to cyno Sdc2. In other embodiments, the antibodies bind to rodent Sdc2 (e.g., a mouse Sdc2). In other embodiments, the antibodies bind to porcine Sdc2 (e.g., a pig Sdc2). In another embodiment, the antibody binds to human Sdc2 and cyno Sdc2. In some embodiments, the antibody binds to a rodent Sdc2 (e.g., a mouse Sdc2). In some embodiments, the antibody binds to a porcine Sdc2 (e.g., a pig Sdc2). In certain embodiments, the antibodies bind to human and/or cyno Sdc2 but does not bind to rodent Sdc2. In some embodiments, the antibody or antigen-binding fragment binds specifically to an epitope which is shared by human, mouse, and porcine Sdc2 proteins or any combination thereof (i.e., human and mouse, mouse and porcine, or porcine and human).

In some embodiments, the anti-Sdc2 antibodies inhibits the binding of Dep-1 to a Sdc2 polypeptide. In some embodiments, the anti-Sdc2 antibodies competes with Dep-1 for binding to a Sdc2 polypeptide. In some embodiments, the anti-Sdc2 antibody binds to a region of human SDC (SEQ ID NO: 25) comprising amino acid residues 123 to 140 of human Sdc2.

In some embodiments of the methods, the anti-Sdc2 antibodies bind to the extracellular domain (ECD) of Sdc2. In certain embodiments, the anti-Sdc2 antibodies bind to an epitope in the ECD of Sdc2. In certain embodiments, the anti-Sdc2 antibodies bind to an epitope in the ECD of Sdc2 to which Dep-1 binds. In some embodiments, the anti-Sdc2 antibodies compete with Dep-1 for binding to a Sdc2 polypeptide. In some embodiments, the anti-Sdc2 antibodies inhibits binding of Dep-1 to a Sdc2 polypeptide. In some embodiments, the anti-Sdc2 antibody blocks binding of Dep-1 to a Sdc2 polypeptide. In certain embodiments, the anti-Sdc2 antibody inhibit Sdc2 activity. In some embodiments, the anti-Sdc2 antibody or antigen binding fragment thereof inhibits binding of Dep-1 to Sdc2.

In some embodiments, the antibody or antigen-binding fragment thereof result in reduced Dep-1 internalization. In some embodiments, the antibody or antigen-binding fragment thereof result in enhanced cell membrane level of Dep-1. In some embodiments, the antibody or antigen-binding fragment thereof result in enhance cell membrane level of Dep-1, without binding directly to the Dep-1 binding site of Sdc2. In some embodiments, the antibody or antigen-binding fragment thereof result in enhanced dephosphorylation of Vascular endothelial growth factor receptor 2 (VEGFR2) protein by Dep-1. In specific embodiments, the antibody or antigen-binding fragment thereof result in enhanced dephosphorylation of VEGFR2 at residue Y951. In specific embodiments, the antibody or antigen-binding fragment thereof inhibits VEGFR2 signaling. In some embodiments, the anti-Sdc2 antibody binds Sdc2, leading to modulation of VEGFR2 or DEP1 signaling and/or molecular activities that result in inhibition of VEGFA-induced vascular permeability.

In some embodiments, the antibody or antigen-binding fragment thereof bind to the Sdc2 extracellular domain and promote stabilization of the endothelial cell junctions, thereby resulting in inhibition of vascular permeability. The invention is also based in part on the discovery that modulation of Sdc2 can be used to treat diseases whose pathogenesis is determined by vessel leakage or edema formation, including AMI among others.

In still other embodiments of the methods, the antibodies competitively block an anti-Sdc2 antibody provided herein from binding to a Sdc2 polypeptide.

In another embodiment of the methods, the antibodies compete for binding to a Sdc2 polypeptide with an anti-Sdc2 antibody provided herein. In some embodiments, the antibodies compete for binding to a Sdc2 polypeptide with a polyclonal anti-Sdc2 antibody Ab3 that binds to Sdc2 at the Dep-1 binding region in Sdc2.

The anti-Sdc2 antibodies provided herein can also be conjugated or recombinantly fused, e.g., to a diagnostic agent or detectable agent. Further provided are compositions comprising an anti-Sdc2 antibody.

In certain aspects, provided herein are methods of managing, preventing, or treating AMI in a subject, comprising administering to a subject an effective amount of binding proteins, such as antibodies that bind to Sdc2. In one embodiment, provided herein is a method of managing AMI in a subject, comprising administering to a subject an effective amount of a Sdc2 binding protein. In one embodiment, provided herein is a method of preventing AMI in a subject, comprising administering to a subject an effective amount of a Sdc2 binding protein. In one embodiment, provided herein is a method of treating AMI in a subject, comprising administering to a subject an effective amount of a Sdc2 binding protein. In a specific embodiment, the Sdc2 binding protein is an antibody that binds to Sdc2. Exemplary Sdc2 antibodies useful in these methods are provided herein.

In certain embodiments, the Sdc2 binding proteins, including antibodies disclosed herein, are antagonist (e.g., inhibiting Sdc2 activity or signaling). In other embodiments, the Sdc2 antibodies inhibit Sdc2 activity. In some embodiments, the binding, competition, and/or signaling is assayed in vitro, e.g., in a cell-based assay. In other embodiments, the binding, competition, and/or signaling is assayed ex vivo, e.g., in an endothelial permeability assay. In other embodiments, the binding and/or signaling is assayed using a sample from a subject (e.g., a human subject). In other embodiments, the binding and/or signaling is assayed in vivo, e.g., in a mouse model of AMI (see, e.g., Example 12). In some embodiments, the activities are exhibited in vitro. In other embodiments, the activities are exhibited in vivo.

The discovery that such binding proteins, including anti-Sdc2 antibodies, inhibiting Sdc2 activity make them viable therapeutics for the treatment of AMI.

In specific embodiments of the various methods provided herein, the binding proteins, such as antibodies that bind to Sdc2, described herein share the common feature of competing with each other for the binding of Sdc2. This competitive inhibition can indicate that each antibody binds to the same region of Sdc2 (e.g., the same epitope), thereby asserting similar effects. In certain embodiments, anti-Sdc2 antibodies provided herein include humanized or fully human anti-Sdc2 antibodies, such as those derived from or based on antibodies 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, and/or R4M-P1-A10. In other embodiments, anti-Sdc2 antibodies provided herein compete for binding with an antibody derived from or based on 20-H19-AB, TP-43327F, TP-43329F, 8-G17-A, 6-N03-A, R3-P3-C11, R4M-P3-E06, R3-P3-E09, R3-P1-C02, R3-P3-A12, R4M-P3-A12, and/or R4M-P1-A10. In some embodiments, the anti-Sdc2 antibodies have CDR sequences as described in Tables 4-8. In certain embodiments, the anti-Sdc2 antibodies bind to the Dep-1 binding region of Sdc2 (e.g., residues 123-140 in the extracellular domain of human Sdc2; see Example 1). Taken together, the results described herein demonstrate that the effects observed for an anti-Sdc2 antibody that is derived from or based on 20-H19-AB, including an antibody having one or more CDRs described in Tables 4-8, can be extrapolated to other anti-Sdc2 antibodies provided herein having the same or similar epitope specificity (e.g., the same or similar CDRs). For example, the activities of antibodies as shown in Examples 4 and 5, for an exemplary humanized anti-Sdc2 antibody, are representative of the activities and effects of the anti-Sdc2 antibodies provided herein.

In some embodiments of the various methods provided herein, the binding proteins such as anti-Sdc2 antibodies may comprise immunoglobulin variable regions which comprise one or more CDRs as described in Tables 4-8. In such binding proteins (e.g., anti-Sdc2 antibodies), the CDRs may be joined with one or more scaffold regions or framework regions (FRs), which orient(s) the CDR(s) such that the proper antigen binding properties of the CDR(s) is achieved.

In certain embodiments, the method comprises administering a Sdc2 binding protein provided herein to the subject in an amount effective for treating the AMI. Also provided herein are methods of managing AMI in a subject. In certain embodiments, the method comprises administering a Sdc2 binding protein provided herein to the subject in an amount effective for managing the AMI. Also provided herein are methods of preventing AMI in a subject. In certain embodiments, the method comprises administering a Sdc2 binding protein provided herein to the subject in an amount effective for preventing the AMI. In some embodiments, the subject has AMI. In other embodiments, the subject is at risk of having AMI. In one embodiment, the subject is a subject in need thereof. In a specific embodiment, the Sdc2 binding protein is a Sdc2 antibody provided herein. In certain embodiments, the Sdc2 binding protein is an antigen binding fragment of a Sdc2 antibody provided herein.

In other embodiments, the anti-Sdc2 antibodies are fully human or humanized antibodies (e.g., comprising human constant regions) that bind Sdc2, including a Sdc2 polypeptide, a Sdc2 polypeptide fragment, a Sdc2 peptide, or a Sdc2 epitope. In certain embodiments, the anti-Sdc2 antibody comprises a VH region, VL region, VH CDR1, VH CDR2, VH CDR3, VL CDR1, VL CDR2, and/or VL CDR3 of any one of the monoclonal antibodies provided herein, such as an amino acid sequence depicted in Tables 1 and 3-8. Accordingly, in some embodiments, the isolated antibody or functional fragment thereof provided herein comprises one, two, and/or three heavy chain CDRs and/or one, two, and/or three light chain CDRs from: (a) the antibody 20-H19-AB, (b) the antibody TP-43327F, (c) the antibody TP-43329F, (d) the antibody 8-G17-A, (e) the antibody 6-N03-A, (f) the antibody R3-P3-C11, (g) the antibody R4M-P3-E06, (h) the antibody R3-P3-E09, (i) the antibody R3-P1-C02, (j) the antibody R3-P3-A12, (k) the antibody R4M-P3-A12, and (l) the antibody R4M-P1-A10, as shown in Tables 1 and 3-8.

In some embodiments, provided herein are methods for preventing, managing or treating AMI is a subject in need thereof, the method comprising administering to the subject a therapeutic effective amount of an antibody that binds to Sdc2 or an antigen binding fragment thereof. In some embodiments, the anti-Sdc2 antibody or an antigen binding fragment thereof is administered systemically to the subject. In some embodiments, the anti-Sdc2 antibody or an antigen binding fragment is administered intravenously to the subject. In some embodiments, the anti-Sdc2 antibody or an antigen binding fragment is administered to the subject by intravenous (IV) bolus. In some embodiments, the Sdc2 antibody is administered to the subject via gene therapy.

Any of the Sdc2 antibodies provided herein are contemplated for use in the methods provided herein. In one embodiment of the methods provided herein, the Sdc2 antibody is 20-H19-AB. In one embodiment, the Sdc2 antibody is derived from 20-H19-AB. In one embodiment, the Sdc2 antibody is an antibody fragment of 20-H19-AB. In one embodiment, the Sdc2 antibody comprises a VH amino acid sequence of 20-H19-AB. In one embodiment, the Sdc2 antibody comprises a VL amino acid sequence of 20-H19-AB. In one embodiment, the Sdc2 antibody comprises a VH amino acid sequence and VL amino acid sequence of 20-H19-AB. In one embodiment, the Sdc2 antibody comprises a VH CDR1, VH CDR2 and VH CDR3 amino acid sequence of 20-H19-AB. In one embodiment, the Sdc2 antibody comprises a VL CDR1, VL CDR2 and VL CDR3 amino acid sequence of 20-H19-AB. In one embodiment, the Sdc2 antibody comprises a VH CDR1, VH CDR2, VH CDR3, VL CDR1, VLCDR2 and VL CDR3 amino acid sequence of 20-H19-AB. In some embodiments, the antibody is an IgG antibody. In some embodiments, the antibody is an IgG1 antibody. In a specific embodiment, the antibody is an IgG4 antibody. Other suitable Sdc2 antibodies are provided in Section 6.7.4 below and elsewhere herein.

In some embodiments, upon administration of the anti-Sdc2 antibody or antigen binding fragment thereof, endothelial permeability in the heart tissue of the subject is reduced. In some embodiments, upon administration of the anti-Sdc2 antibody or antigen binding fragment thereof to the subject, endothelia permeability in the heart tissue is reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100%. In some embodiments, the endothelial cell is on a blood vessel. In some embodiments, the endothelial cell is in a heart tissue. In some embodiments, the endothelial cell is on a blood vessel in the heart. In some embodiments, the blood vessel is in a heart. In some embodiments, the blood vessel is a heart vein. In some embodiments, the blood vessel is a heart artery. In some embodiments, the blood vessel is part of the left anterior descending coronary artery (LAD). In some embodiments, the endothelial permeability is VEGFA-induced endothelial permeability. In some embodiments, the endothelial permeability is measured by an Evans Blue assay. In some embodiments, the endothelial permeability is measured by a Dextran perfusion assay. In some embodiments, the endothelial cell expresses Sdc2. In some embodiments, reduction of endothelial permeability occurs within about 12 hours, within about 24 hours, within about 36 hours, or within about 48 hours after administration of the anti-Sdc2 antibody to the subject. In some embodiments, reduction of endothelial permeability occurs within about 12 hours, within about 24 hours, within about 36 hours, or within about 48 hours after the subject suffered from an episode of AMI and is treated with the anti-Sdc2 antibody. In some embodiments, reduction of endothelial permeability lasts for at least about 72 hours, at least about 1 week, at least about 2 weeks, at least about 3 weeks, at least about 4 weeks, at least about 5 weeks, at least about 6 weeks after administration of the anti-Sdc2 antibody to the subject. In some embodiments, reduction of endothelial permeability lasts for at least about 72 hours, at least about 1 week, at least about 2 weeks, at least about 3 weeks, at least about 4 weeks, at least about 5 weeks, at least about 6 weeks after the subject suffered from an episode of AMI and is treated with anti-Sdc2 antibody.

In some embodiments, upon administering of the antibody or antigen binding fragment thereof, Dep-1 expression on surface of cells in a heart tissue is upregulated. In some embodiments, upon administering of the antibody or antigen binding fragment thereof, dephosphorylation of vascular endothelial growth factor receptor 2 (VEGFR2) protein at residue Y951 is enhanced in cells in a heart tissue.

In some embodiments, upon administration of the anti-Sdc2 antibody or antigen binding fragment thereof, vascular permeability in the heart tissue of the subject is reduced. In some embodiments, upon administration of the anti-Sdc2 antibody or antigen binding fragment thereof to the subject, vascular permeability in the heart tissue is reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100%. In some embodiments, the endothelial cell is on a blood vessel. In some embodiments, the blood vessel is a heart vein. In some embodiments, the blood vessel is a heart artery. In some embodiments, the blood vessel is part of the left anterior descending coronary artery (LAD). In some embodiments, the vascular permeability is measured by an Evans Blue assay. In some embodiments, the vascular permeability is measured by a Dextran perfusion assay. In some embodiments, the endothelial cell expresses Sdc2. In some embodiments, reduction of vascular permeability occurs within about 12 hours, within about 24 hours, within about 36 hours, or within about 48 hours after administration of the anti-Sdc2 antibody to the subject. In some embodiments, reduction of vascular permeability occurs within about 12 hours, within about 24 hours, within about 36 hours, or within about 48 hours after the subject suffered from an episode of AMI and is treated with the anti-Sdc2 antibody. In some embodiments, reduction of vascular permeability lasts for at least about 72 hours, at least about 1 week, at least about 2 weeks, at least about 3 weeks, at least about 4 weeks, at least about 5 weeks, at least about 6 weeks after administration of the anti-Sdc2 antibody to the subject. In some embodiments, reduction of vascular permeability lasts for at least about 72 hours, at least about 1 week, at least about 2 weeks, at least about 3 weeks, at least about 4 weeks, at least about 5 weeks, at least about 6 weeks after the subject suffered from an episode of AMI and is treated with anti-Sdc2 antibody.

In some embodiments, the subject has an infarct in the heart associated with the AMI. In some embodiments, upon administration of the anti-Sdc2 antibody or antigen binding fragment thereof, the infarct size in the heart of the subject is reduced. In some embodiments, upon administration of the anti-Sdc2 antibody or antigen binding fragment thereof to the subject, the infarct size in the heart of the subject is reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100%. In some embodiments, the reduction in the infarct size occurs within about 12 hours after the subject suffered from an episode of AMI and treated with the anti-Sdc2 antibody. In some embodiments, the reduction in the infarct size occurs within about 24 hours after the subject suffered from an episode of AMI and treated with the anti-Sdc2 antibody. In some embodiments, the reduction in the infarct size occurs within about 36 hours after the subject suffered from an episode of AMI and treated with the anti-Sdc2 antibody. In some embodiments, the reduction in the infarct size occurs within about 48 hours after the subject suffered from an episode of AMI and treated with the anti-Sdc2 antibody. In some embodiments, the reduction in the infarct size lasts for at least about 1 week, at least about 2 weeks, at least about 3 weeks, at least about 4 weeks, at least about 5 weeks, or at least about 6 weeks after the subject suffered from an episode of AMI and treated with the anti-Sdc2 antibody.

In some embodiments, the subject has an edema in the heart associated with the AMI. In some embodiments, upon administration of the anti-Sdc2 antibody or antigen binding fragment thereof, the edema volume is reduced. In some embodiments, the edema volume is reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100%. In some embodiments, the reduction in the edema volume occurs within about 12 hours after the subject suffered from an episode of AMI and treated with the anti-Sdc2 antibody. In some embodiments, the reduction in the edema volume occurs within about 24 hours after the subject suffered from an episode of AMI and treated with the anti-Sdc2 antibody. In some embodiments, the reduction in the edema volume occurs within about 36 hours after the subject suffered from an episode of AMI and treated with the anti-Sdc2 antibody. In some embodiments, the reduction in the edema volume occurs within about 48 hours after the subject suffered from an episode of AMI and treated with the anti-Sdc2 antibody. In some embodiments, the reduction in the edema volume lasts for at least about 1 week, at least about 2 weeks, at least about 3 weeks, at least about 4 weeks, at least about 5 weeks, or at least about 6 weeks after the subject suffered from an episode of AMI and treated with the anti-Sdc2 antibody.

In some embodiments, upon administering of the antibody or antigen binding fragment thereof, inflammation in a heart tissue of the subject is reduced. In some embodiments, expression of one or more inflammatory marker in the heart tissue is reduced; optionally wherein the inflammatory marker is selected from pro-inflammatory cytokines and immune cell surface proteins; optionally wherein the inflammatory marker is selected from CD11b, GM-CSF, MIG, CCL11, IL-3, IL-6, TNF-α, and MCP1 (CCL2). In some embodiments, reduction of expression of the one or more inflammatory marker occurred within about 24 hours, within about 36 hours, or within about 72 hours after administration of the antibody or antigen binding fragment thereof.

In some embodiments, the subject has infection in the heart tissue associated with the AMI. In some embodiments, upon administration of the anti-Sdc2 antibody or antigen binding fragment thereof, the infection in the heart tissue of the subject is reduced. In some embodiments, upon administration of the anti-Sdc2 antibody or antigen binding fragment thereof to the subject, expression of one or more pro-inflammatory cytokines in the heart tissue is reduced. In some embodiments, expression of one or more pro-inflammatory cytokines in the heart tissue is reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100%. In some embodiments, the cytokine is IL-1, IL-2, IL-3, IL-6, IL-12, IL-17, IL-22, IL-23, GM-CSF, MIG, CCL11, IFN-γ, or a combination thereof. In some embodiments, the heart tissue is in the left ventricular of the heart. In some embodiments, the heart tissue is in the right atrium of the heart. In some embodiments, the reduction in the expression of one or more pro-inflammatory cytokines occurs within about 12 hours, within about 24 hours, about 36 hours, or about 48 hours after administration of the anti-Sdc2 antibody. In some embodiments, the reduction in the expression of one or more pro-inflammatory cytokines occurs within about 12 hours, within about 24 hours, about 36 hours, or about 48 hours after the subject suffers an episode of AMI and is treated with the anti-Sdc2 antibody. In some embodiments, the reduction in the expression of one or more pro-inflammatory cytokines last for at least about 1 week, at least about 2 weeks, at least about 3 weeks, at least about 4 weeks, at least about 5 weeks, or at least about 6 weeks after administration of the anti-Sdc2 antibody. In some embodiments, the reduction in the expression of one or more pro-inflammatory cytokines last for at least about 1 week, at least about 2 weeks, at least about 3 weeks, at least about 4 weeks, at least about 5 weeks, or at least about 6 weeks after the subject suffers an episode of AMI and is treated with the anti-Sdc2 antibody.

In some embodiments, upon administration of the anti-Sdc2 antibody or antigen binding fragment thereof to the subject, recruitment of immune cells to the heart tissue is reduced in the subject. In some embodiments, recruitment of immune cells to the heart tissue is reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100%. In some embodiments, the immune cell is selected from T cells, B cells, natural killer cells, neutrophils, mast cells, macrophages, antigen-presenting cells (APC), basophils, and eosinophils. In some embodiments, the heart tissue is in the left ventricular. In some embodiments, the reduction in the recruitment of one or more immune cells last for at least about 1 week, at least about 2 weeks, at least about 3 weeks, or at least about 4 weeks, at least about 5 weeks, or at least about 6 weeks after the subject suffers an episode of AMI and treated with the anti-Sdc2 antibody.

In some embodiments, upon administration of the anti-Sdc2 antibody or antigen binding fragment thereof, expression of one or more T cell markers in the heart tissue is reduced. In some embodiments, expression of one or more T cell markers in the heart tissue is reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100%. In some embodiments, the T cell marker is selected from TRBC2, CD3D, CD3G, CD3E, LTB, IL7R, LEF1, GZMK, TRAC, HOPX, IFIT3, CERK, GIMAP3, CXCR6, SATB1, PTPRCAP, CD69, TRBC1, CD2, GDPD3, IL2RA, CCL5, MYB, GZMA, CD52, CCL4, GIMAP2, SYT3, NOTCH3, SEMA6D, DKK3, PMCH, ITK, GEM, MAFF, TGIF1, RORA, TNFAIP3, CREM, PXDC1, NABP1, FAM110A, EEF1B2P3, PFN1P1, IL32, CXCR4, SEPT1, BCL2, CYTL1, CTSW, PTPN22, TXK, TRAF1, CD8B, BATF3, GZMH, LAG3, CD8B1, GZMB, SH2D1A, MYO1G, FMNL1, S1PR4, CD247, MS4A4C, GIMAP5, CD28, CD160, TCRG-C2, TCRG-C1, TCRG-C4, TRDC, H2-T3, H2-T10, RHOH, KLRB1, CCR2, IL2RB, CD163L1, TRDV4, MBD2, ICOS, IL18R1, TNFRSF4, CCL20, CLEC2D, CD8A, CD6, S100A4, LCK, CD81, THY1, LAT, SKAP1, TCF7, CCL4L2, PYHIN1, JUNB, DUSP2, IFNG, BRAF, CCL3, CCL6, CD4, CD7, H2-Q7, and CCR7.

In some embodiments, upon administration of the anti-Sdc2 antibody or antigen binding fragment thereof, expression of one or more B cell markers in the heart tissue is reduced. In some embodiments, expression of one or more B cell markers in the heart tissue is reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100%. In some embodiments, the B cell marker is selected from PXK, CD19, MS4A1, CD74, CD79A, PTPRC, IGHD, IGHM, HLA-DRA, PAX5, BANK1, CR2, CD22, FCER2, CD79B, IGLL1, SPN, B3GAT1, CD72, LY6D, IGLC1, CD5, CD40, CD69, CD70, CD86, TNFRSF9, SDC1, TNFSF4, TNFRSF13B, TNFRSF13C, PDCD1, RASGRP3, HLA-DQA1, FLI1, CD14, SEMA6D, LAIR1, IFIT3, DNTT, CD24, CD27, MUM1, JCHAIN, MZB1, H2-DMB2, FCMR, EDEM1, VPREB3, H2-OB, POU2AF1, CRELD2, DERL3, SIGLECG, RALGPS2, FCHSD2, POLD4, CMAH, TNFRSF17, HVCN1, TRP53INP1, FCRLA, EDEM2, BLNK, H2-OA, TXNDC11, BTLA, SMAP2, SCD1, FAM46C, FKBP11, SEC61A1, SPCS3, SPIB, EAF2, CXCR4, BIRC3, IGLC2, IGLC3, IL21R, IGKC, IGLV1, VPREB1, VPREB2, LRMP, KLHL6, SLAMF6, FAM129C, BST1, MSH5, DOK3, BACH2, FCER2A, IGHG3, IGHG4, IGHA, CD38, EBF1, BCL11A, CCR7, CD55, CD52, TLR9, SWAP70, HMGA1, CD2, CD80, LTB, MME, and IGHG1.

In some embodiments, upon administration of the anti-Sdc2 antibody or antigen binding fragment thereof, expression of one or more natural killer cell markers in the heart tissue is reduced. In some embodiments, expression of one or more natural killer cell markers in the heart tissue is reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100%. In some embodiments, the natural killer cell marker is selected from NCAM1, IL2RB, CD44, IL12RB2, CXCR4, ZFP683, SLAMF7, TCF7, STYK1, S1PR1, GATA3, TBX21, ZBTB16, PRF1, TNFRSF8, GZMB, NR4A1, MAP3K8, EGR1, ITGB2, RORA, KLRB1, IL12RB1, and IL17RA.

In some embodiments, upon administration of the anti-Sdc2 antibody or antigen binding fragment thereof, expression of one or more macrophage markers in the heart tissue is reduced. In some embodiments, expression of one or more macrophage markers in the heart tissue is reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100%. In some embodiments, the macrophage marker is selected from CD11b, CD68, FCGR1, NAAA, JAML, TYROBP, TREM2, H2-DMA, CXCL16, MGL2, CLEC4A2, CCL12, PARP14, SEPP1, MARCH1, FGL2, LYZ2, WFDC17, CPM, CHIT1, TGFBR1, SLAMF9, SCIMP, LILRA5, C3AR1, FGD2, RAB7B, RBPJ, SLCO2B1, EGLN3, CLEC4D, ADAM8, ARL11, MMP12, VSIG4, RETNLA, RAB20, SNX20, FMNL1, GPR132, NCEH1, CCL24, CD300A, CCL7, IRF5, MYO1G, DUSP5, GPR171, SAMSN1, NR4A3, HILPDA, SLC37A2, LPCAT2, IFNAR2, MS4A7, HPGD, CD5L, LILR4B, CYTH4, CLEC4N, CD209F, CSF1R, FGR, CYBB, CD200, CD200R1, GATA6, H2-AB1, H2-EB1, MAF, AIF1, ADGRE5, CLEC4A3, CLEC4A1, SLC15A3, AKR1B3, CYP27A1, ITGAL, ITGAM, CD14, FUT4, FCGR3A, CD33, FCGR1A, CD80, LILRB4, CD86, CD163, CCR5, TLR2, TLR4, ADGRE1 (f4/80), GPR34, CCL9, FABP4, S100A8, F13A1, CD83, STAB1, MRC1, CCL6, GPNMB, LYVE1, PLTP, MS4A4A, MS4A6A, FPR1, CD180, GDF15, HFE, TNF, CCR2, CD209A, C5ARI, CXCL2, CCL2, IL1B, AHR, CCR7, DNASE1L3, CXCL1, CCL22, S100A4, MMP9, NRP2, CTSK, CD36, HPGDS, SLC11A1, CCL3, CLEC7A, CCL5, CD3E, CD19, CD74, LYZ1, LGALS3, UCP2, TREML4, IL4RA, ITGAX, LY6C1, PPARG, SIGLECF, RGS1, DAB2, P2RY6, CLEC10A, ADGRE4, RUNX3, SYK, CX3CR1, and SLAMF7. In specific embodiments, upon administration of the anti-Sdc2 antibody or antigen binding fragment thereof, CD11b expression level in the heart tissue is reduced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100%. In some embodiments, the reduction of CD11b expression level last for at least about 1 week, at least about 2 weeks, at least about 3 weeks, at least about 4 weeks, at least about 5 weeks, or at least about 6 weeks after the subject suffered an episode of AMI and treated with the anti-Sdc2 antibody.

In some embodiments, upon administration of the anti-Sdc2 antibody or antigen binding fragment thereof to the subject suffered from AMI, post-infarction LV function of the heart of the subject is enhanced. In some embodiments, upon administration of the anti-Sdc2 antibody or antigen binding fragment thereof to the subject, post-infarction LV function of the subject's heart is enhanced by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or at least about 100%. In some embodiments, the enhancement of the subject's post-infarction LV function occurs within about 1 week, within about 2 weeks, within about 3 weeks, without about 4 weeks, within about 5 weeks, or within about 6 weeks after the subject suffered an episode of AMI and treated with the anti-Sdc2 antibody.

In some embodiments, upon administering of the antibody or antigen binding fragment thereof, the LVEF of the heart of the subject is enhanced. In some embodiments, upon administering of the antibody or antigen binding fragment thereof, the cardiac output of the heart of the subject is enhanced. In some embodiments, upon administering of the antibody or antigen binding fragment thereof, the LV end diastolic diameter (LVEDD) of the heart of the subject is reduced. In some embodiments, upon administering of the antibody or antigen binding fragment thereof, the LV end systolic diameter (LVESD) of the heart of the subject is reduced. In some embodiments, upon administering of the antibody or antigen binding fragment thereof, the LV end diastolic volume of the heart of the subject is reduced. In some embodiments, upon administering of the antibody or antigen binding fragment thereof, the LV end systolic volume of the heart of the subject is reduced. In some embodiments, upon administering of the antibody or antigen binding fragment thereof, the LV mass of the heart of the subject is reduced. In some embodiments, upon administering of the antibody or antigen binding fragment thereof, the fractional shortening of the heart of the subject is enhanced. In some embodiments, upon administering of the antibody or antigen binding fragment thereof, the ejection fraction of the heart of the subject is enhanced. In some embodiments, upon administering of the antibody or antigen binding fragment thereof, the risk or duration of post-infarct ventricular tachycardia (VT) of the subject is reduced. In some embodiments, upon administering of the antibody or antigen binding fragment thereof, the risk for the subject to have a heart failure is reduced; optionally where the risk is having the heart failure within 1 month, 2 months, or three months following an episode of AMI. In some embodiments, post-infarct VT of the subject is measured by an electrocardiogram with programmed stimulation of the subject's heart. In one embodiment, an increase in the number of stimuli for inducing the VT indicates a reduced risk of post-infarct VT in the subject. In one embodiment, a reduced duration of induced VT under a hypokalemic condition indicates a reduced risk of post-infarct VT in the subject. In one embodiment, a longer cycle length of induced VT indicates a reduced risk of post-infarct VT in the subject.

In some embodiments, upon administration of the anti-Sdc2 antibody or antigen binding fragment thereof to the subject suffered from AMI, cardiac output is enhanced in the subject. In some embodiments, upon administration of the anti-Sdc2 antibody or antigen binding fragment thereof to the subject suffered from AMI, end diastolic LV internal diameter (LVIDd) or LV end diastolic diameter (LVEDD) is reduced in the subject. In some embodiments, upon administration of the anti-Sdc2 antibody or antigen binding fragment thereof to the subject suffered from AMI, end systolic LV internal diameter (LVIDs) or LV end systolic diameter (LVESD) is reduced in the subject. In some embodiments, upon administration of the anti-Sdc2 antibody or antigen binding fragment thereof to the subject suffered from AMI, the ejection fraction of the heart of the subject is enhanced. In some embodiments, upon administration of the anti-Sdc2 antibody or antigen binding fragment thereof to the subject suffered from AMI, fractional shortening (FS) of the heart of the subject is enhanced. In some embodiments, upon administration of the anti-Sdc2 antibody or antigen binding fragment thereof to the subject suffered from AMI, the LV mass of the heart of a subject is reduced. In some embodiments, upon administration of the anti-Sdc2 antibody or antigen binding fragment thereof to the subject suffered from AMI, the LV end diastolic volume of the heart is reduced. In some embodiments, upon administration of the anti-Sdc2 antibody or antigen binding fragment thereof to the subject suffered from AMI, the LV end systolic volume of the heart of the subject is reduced. In some embodiments, upon administration of the anti-Sdc2 antibody or antigen binding fragment thereof to the subject suffered from AMI, the risk of the subject to suffer post-infarction ventricular tachycardia (VT) is reduced.

In some embodiments, upon administration of the anti-Sdc2 antibody or antigen binding fragment thereof to the subject suffered from an episode of AMI, the risk of the subject to suffer from heart failure after the episode of AMI is reduced. In some embodiments, the risk of the subject to suffer from heart failure within 1 month, within 2 months, with 3 months, within 6 months, within 1 year or after 1 year following the episode of AMI is reduced.

In some embodiments of the methods provided herein, the subject is a human suffering from or at risk of developing AMI.

Moreover, an anti-Sdc2 antibody or fragment thereof can be administered to a non-human mammal expressing Sdc2 with which the antibody cross-reacts (e.g., a primate) for veterinary purposes or as an animal model of human disease. Regarding the latter, such animal models may be useful for evaluating the therapeutic efficacy of antibodies of the present disclosure (e.g., testing of dosages and time courses of administration).

In some embodiments of the various methods provided herein, the method further comprises administering a therapeutically effective amount of a second active agent or a support care therapy. Second active agents can be large molecules (e.g., proteins) or small molecules (e.g., synthetic inorganic, organometallic, or organic molecules). In some embodiments, the second active agents are small molecules that can alleviate adverse effects associated with the administration of an antibody provided herein. However, like some large molecules, many are believed to be capable of providing a synergistic effect when administered with (e.g., before, after or simultaneously) an antibody provided herein. Examples of small molecule second active agents include, but are not limited to, anti-cancer agents, anti-inflammatory agents, immunosuppressive agents, and steroids.

### 6.7.4 Exemplary Antibodies Useful in the Methods Provided Herein

Exemplary Sdc2 antibodies are provided herein, and any of the exemplified Sdc2 antibodies can be used in a method provided herein. In one embodiment of the methods provided herein, the Sdc2 antibody is 20-H19-AB. In one embodiment, the Sdc2 antibody is derived from 20-H19-AB. In one embodiment, the Sdc2 antibody is an antibody fragment of 20-H19-AB. In one embodiment, the Sdc2 antibody comprises a VH amino acid sequence of 20-H19-AB. In one embodiment, the Sdc2 antibody comprises a VL amino acid sequence of 20-H19-AB. In one embodiment, the Sdc2 antibody comprises a VH amino acid sequence and VL amino acid sequence of 20-H19-AB. In one embodiment, the Sdc2 antibody comprises a VH CDR1, VH CDR2 and VH CDR3 amino acid sequence of 20-H19-AB. In one embodiment, the Sdc2 antibody comprises a VL CDR1, VL CDR2 and VL CDR3 amino acid sequence of 20-H19-AB. In one embodiment, the Sdc2 antibody comprises a VH CDR1, VH CDR2, VH CDR3, VL CDR1, VLCDR2 and VL CDR3 amino acid sequence of 20-H19-AB. In one embodiment, the Sdc2 antibody is used for the treatment of a stroke in a subject, as provided herein. In one embodiment, the Sdc2 antibody is used for the treatment of ischemic stroke in a subject, as provided herein. In one embodiment, the Sdc2 antibody is used for the treatment of a neovascular eye disease in a subject, as provided herein. In one embodiment, the Sdc2 antibody is used for the treatment of AMD in a subject, as provided herein. In one embodiment, the Sdc2 antibody is used for the treatment of a cardiovascular disease in a subject, as provided herein. In one embodiment, the Sdc2 antibody is used for the treatment of acute myocardial infarction in a subject, as provided herein.

In one embodiment of the methods provided herein, the Sdc2 antibody is TP-43327F. In one embodiment, the Sdc2 antibody is derived from TP-43327F. In one embodiment, the Sdc2 antibody is an antibody fragment of TP-43327F. In one embodiment, the Sdc2 antibody comprises a VH amino acid sequence of TP-43327F. In one embodiment, the Sdc2 antibody comprises a VL amino acid sequence of TP-43327F. In one embodiment, the Sdc2 antibody comprises a VH amino acid sequence and VL amino acid sequence of TP-43327F. In one embodiment, the Sdc2 antibody comprises a VH CDR1, VH CDR2 and VH CDR3 amino acid sequence of TP-43327F. In one embodiment, the Sdc2 antibody comprises a VL CDR1, VL CDR2 and VL CDR3 amino acid sequence of TP-43327F. In one embodiment, the Sdc2 antibody comprises a VH CDR1, VH CDR2, VH CDR3, VL CDR1, VLCDR2 and VL CDR3 amino acid sequence of TP-43327F. In one embodiment, the Sdc2 antibody is used for the treatment of a stroke in a subject, as provided herein. In one embodiment, the Sdc2 antibody is used for the treatment of ischemic stroke in a subject, as provided herein. In one embodiment, the Sdc2 antibody is used for the treatment of a neovascular eye disease in a subject, as provided herein. In one embodiment, the Sdc2 antibody is used for the treatment of AMD in a subject, as provided herein. In one embodiment, the Sdc2 antibody is used for the treatment of a cardiovascular disease in a subject, as provided herein. In one embodiment, the Sdc2 antibody is used for the treatment of acute myocardial infarction in a subject, as provided herein.

In one embodiment of the methods provided herein, the Sdc2 antibody is TP-43329F. In one embodiment, the Sdc2 antibody is derived from TP-43329F. In one embodiment, the Sdc2 antibody is an antibody fragment of TP-43329F. In one embodiment, the Sdc2 antibody comprises a VH amino acid sequence of TP-43329F. In one embodiment, the Sdc2 antibody comprises a VL amino acid sequence of TP-43329F. In one embodiment, the Sdc2 antibody comprises a VH amino acid sequence and VL amino acid sequence of TP-43329F. In one embodiment, the Sdc2 antibody comprises a VH CDR1, VH CDR2 and VH CDR3 amino acid sequence of TP-43329F. In one embodiment, the Sdc2 antibody comprises a VL CDR1, VL CDR2 and VL CDR3 amino acid sequence of TP-43329F. In one embodiment, the Sdc2 antibody comprises a VH CDR1, VH CDR2, VH CDR3, VL CDR1, VLCDR2 and VL CDR3 amino acid sequence of TP-43329F. In one embodiment, the Sdc2 antibody is used for the treatment of a stroke in a subject, as provided herein. In one embodiment, the Sdc2 antibody is used for the treatment of ischemic stroke in a subject, as provided herein. In one embodiment, the Sdc2 antibody is used for the treatment of a neovascular eye disease in a subject, as provided herein. In one embodiment, the Sdc2 antibody is used for the treatment of AMD in a subject, as provided herein. In one embodiment, the Sdc2 antibody is used for the treatment of a cardiovascular disease in a subject, as provided herein. In one embodiment, the Sdc2 antibody is used for the treatment of acute myocardial infarction in a subject, as provided herein.

In one embodiment of the methods provided herein, the Sdc2 antibody is 8-G17A. In one embodiment, the Sdc2 antibody is derived from 8-G17A. In one embodiment, the Sdc2 antibody is an antibody fragment of 8-G17A. In one embodiment, the Sdc2 antibody comprises a VH amino acid sequence of 8-G17A. In one embodiment, the Sdc2 antibody comprises a VL amino acid sequence of 8-G17A. In one embodiment, the Sdc2 antibody comprises a VH amino acid sequence and VL amino acid sequence of 8-G17A. In one embodiment, the Sdc2 antibody comprises a VH CDR1, VH CDR2 and VH CDR3 amino acid sequence of 8-G17A. In one embodiment, the Sdc2 antibody comprises a VL CDR1, VL CDR2 and VL CDR3 amino acid sequence of 8-G17A. In one embodiment, the Sdc2 antibody comprises a VH CDR1, VH CDR2, VH CDR3, VL CDR1, VLCDR2 and VL CDR3 amino acid sequence of 8-G17A. In one embodiment, the Sdc2 antibody is used for the treatment of a stroke in a subject, as provided herein. In one embodiment, the Sdc2 antibody is used for the treatment of ischemic stroke in a subject, as provided herein. In one embodiment, the Sdc2 antibody is used for the treatment of a neovascular eye disease in a subject, as provided herein. In one embodiment, the Sdc2 antibody is used for the treatment of AMD in a subject, as provided herein. In one embodiment, the Sdc2 antibody is used for the treatment of a cardiovascular disease in a subject, as provided herein. In one embodiment, the Sdc2 antibody is used for the treatment of acute myocardial infarction in a subject, as provided herein.

In one embodiment of the methods provided herein, the Sdc2 antibody is 6-N03-A. In one embodiment, the Sdc2 antibody is derived from 6-N03-A. In one embodiment, the Sdc2 antibody is an antibody fragment of 6-N03-A. In one embodiment, the Sdc2 antibody comprises a VH amino acid sequence of 6-N03-A. In one embodiment, the Sdc2 antibody comprises a VL amino acid sequence of 6-N03-A. In one embodiment, the Sdc2 antibody comprises a VH amino acid sequence and VL amino acid sequence of 6-N03-A. In one embodiment, the Sdc2 antibody comprises a VH CDR1, VH CDR2 and VH CDR3 amino acid sequence of 6-N03-A. In one embodiment, the Sdc2 antibody comprises a VL CDR1, VL CDR2 and VL CDR3 amino acid sequence of 6-N03-A. In one embodiment, the Sdc2 antibody comprises a VH CDR1, VH CDR2, VH CDR3, VL CDR1, VLCDR2 and VL CDR3 amino acid sequence of 6-N03-A. In one embodiment, the Sdc2 antibody is used for the treatment of a stroke in a subject, as provided herein. In one embodiment, the Sdc2 antibody is used for the treatment of ischemic stroke in a subject, as provided herein. In one embodiment, the Sdc2 antibody is used for the treatment of a neovascular eye disease in a subject, as provided herein. In one embodiment, the Sdc2 antibody is used for the treatment of AMD in a subject, as provided herein. In one embodiment, the Sdc2 antibody is used for the treatment of a cardiovascular disease in a subject, as provided herein. In one embodiment, the Sdc2 antibody is used for the treatment of acute myocardial infarction in a subject, as provided herein.

In one embodiment of the methods provided herein, the Sdc2 antibody is R3-P3-C11. In one embodiment, the Sdc2 antibody is derived from R3-P3-C11. In one embodiment, the Sdc2 antibody is an antibody fragment of R3-P3-C11. In one embodiment, the Sdc2 antibody comprises a VH amino acid sequence of R3-P3-C11. In one embodiment, the Sdc2 antibody comprises a VL amino acid sequence of R3-P3-C11. In one embodiment, the Sdc2 antibody comprises a VH amino acid sequence and VL amino acid sequence of R3-P3-C11. In one embodiment, the Sdc2 antibody comprises a VH CDR1, VH CDR2 and VH CDR3 amino acid sequence of R3-P3-C11. In one embodiment, the Sdc2 antibody comprises a VL CDR1, VL CDR2 and VL CDR3 amino acid sequence of R3-P3-C11. In one embodiment, the Sdc2 antibody comprises a VH CDR1, VH CDR2, VH CDR3, VL CDR1, VLCDR2 and VL CDR3 amino acid sequence of R3-P3-C11. In one embodiment, the Sdc2 antibody is used for the treatment of a stroke in a subject, as provided herein. In one embodiment, the Sdc2 antibody is used for the treatment of ischemic stroke in a subject, as provided herein. In one embodiment, the Sdc2 antibody is used for the treatment of a neovascular eye disease in a subject, as provided herein. In one embodiment, the Sdc2 antibody is used for the treatment of AMD in a subject, as provided herein. In one embodiment, the Sdc2 antibody is used for the treatment of a cardiovascular disease in a subject, as provided herein. In one embodiment, the Sdc2 antibody is used for the treatment of acute myocardial infarction in a subject, as provided herein.

In one embodiment of the methods provided herein, the Sdc2 antibody is R4M-P3-E06. In one embodiment, the Sdc2 antibody is derived from R4M-P3-E06. In one embodiment, the Sdc2 antibody is an antibody fragment of R4M-P3-E06. In one embodiment, the Sdc2 antibody comprises a VH amino acid sequence of R4M-P3-E06. In one embodiment, the Sdc2 antibody comprises a VL amino acid sequence of R4M-P3-E06. In one embodiment, the Sdc2 antibody comprises a VH amino acid sequence and VL amino acid sequence of R4M-P3-E06. In one embodiment, the Sdc2 antibody comprises a VH CDR1, VH CDR2 and VH CDR3 amino acid sequence of R4M-P3-E06. In one embodiment, the Sdc2 antibody comprises a VL CDR1, VL CDR2 and VL CDR3 amino acid sequence of R4M-P3-E06. In one embodiment, the Sdc2 antibody comprises a VH CDR1, VH CDR2, VH CDR3, VL CDR1, VLCDR2 and VL CDR3 amino acid sequence of R4M-P3-E06. In one embodiment, the Sdc2 antibody is used for the treatment of a stroke in a subject, as provided herein. In one embodiment, the Sdc2 antibody is used for the treatment of ischemic stroke in a subject, as provided herein. In one embodiment, the Sdc2 antibody is used for the treatment of a neovascular eye disease in a subject, as provided herein. In one embodiment, the Sdc2 antibody is used for the treatment of AMD in a subject, as provided herein. In one embodiment, the Sdc2 antibody is used for the treatment of a cardiovascular disease in a subject, as provided herein. In one embodiment, the Sdc2 antibody is used for the treatment of acute myocardial infarction in a subject, as provided herein.

In one embodiment of the methods provided herein, the Sdc2 antibody is R3-P3-E09. In one embodiment, the Sdc2 antibody is derived from R3-P3-E09. In one embodiment, the Sdc2 antibody is an antibody fragment of R3-P3-E09. In one embodiment, the Sdc2 antibody comprises a VH amino acid sequence of R3-P3-E09. In one embodiment, the Sdc2 antibody comprises a VL amino acid sequence of R3-P3-E09. In one embodiment, the Sdc2 antibody comprises a VH amino acid sequence and VL amino acid sequence of R3-P3-E09. In one embodiment, the Sdc2 antibody comprises a VH CDR1, VH CDR2 and VH CDR3 amino acid sequence of R3-P3-E09. In one embodiment, the Sdc2 antibody comprises a VL CDR1, VL CDR2 and VL CDR3 amino acid sequence of R3-P3-E09. In one embodiment, the Sdc2 antibody comprises a VH CDR1, VH CDR2, VH CDR3, VL CDR1, VLCDR2 and VL CDR3 amino acid sequence of R3-P3-E09. In one embodiment, the Sdc2 antibody is used for the treatment of a stroke in a subject, as provided herein. In one embodiment, the Sdc2 antibody is used for the treatment of ischemic stroke in a subject, as provided herein. In one embodiment, the Sdc2 antibody is used for the treatment of a neovascular eye disease in a subject, as provided herein. In one embodiment, the Sdc2 antibody is used for the treatment of AMD in a subject, as provided herein. In one embodiment, the Sdc2 antibody is used for the treatment of a cardiovascular disease in a subject, as provided herein. In one embodiment, the Sdc2 antibody is used for the treatment of acute myocardial infarction in a subject, as provided herein.

In one embodiment of the methods provided herein, the Sdc2 antibody is R3-P1-C02. In one embodiment, the Sdc2 antibody is derived from R3-P1-C02. In one embodiment, the Sdc2 antibody is an antibody fragment of R3-P1-C02. In one embodiment, the Sdc2 antibody comprises a VH amino acid sequence of R3-P1-C02. In one embodiment, the Sdc2 antibody comprises a VL amino acid sequence of R3-P1-C02. In one embodiment, the Sdc2 antibody comprises a VH amino acid sequence and VL amino acid sequence of R3-P1-C02. In one embodiment, the Sdc2 antibody comprises a VH CDR1, VH CDR2 and VH CDR3 amino acid sequence of R3-P1-C02. In one embodiment, the Sdc2 antibody comprises a VL CDR1, VL CDR2 and VL CDR3 amino acid sequence of R3-P1-C02. In one embodiment, the Sdc2 antibody comprises a VH CDR1, VH CDR2, VH CDR3, VL CDR1, VLCDR2 and VL CDR3 amino acid sequence of R3-P1-C02. In one embodiment, the Sdc2 antibody is used for the treatment of a stroke in a subject, as provided herein. In one embodiment, the Sdc2 antibody is used for the treatment of ischemic stroke in a subject, as provided herein. In one embodiment, the Sdc2 antibody is used for the treatment of a neovascular eye disease in a subject, as provided herein. In one embodiment, the Sdc2 antibody is used for the treatment of AMD in a subject, as provided herein. In one embodiment, the Sdc2 antibody is used for the treatment of a cardiovascular disease in a subject, as provided herein. In one embodiment, the Sdc2 antibody is used for the treatment of acute myocardial infarction in a subject, as provided herein.

In one embodiment of the methods provided herein, the Sdc2 antibody is R3-P3-A12. In one embodiment, the Sdc2 antibody is derived from R3-P3-A12. In one embodiment, the Sdc2 antibody is an antibody fragment of R3-P3-A12. In one embodiment, the Sdc2 antibody comprises a VH amino acid sequence of R3-P3-A12. In one embodiment, the Sdc2 antibody comprises a VL amino acid sequence of R3-P3-A12. In one embodiment, the Sdc2 antibody comprises a VH amino acid sequence and VL amino acid sequence of R3-P3-A12. In one embodiment, the Sdc2 antibody comprises a VH CDR1, VH CDR2 and VH CDR3 amino acid sequence of R3-P3-A12. In one embodiment, the Sdc2 antibody comprises a VL CDR1, VL CDR2 and VL CDR3 amino acid sequence of R3-P3-A12. In one embodiment, the Sdc2 antibody comprises a VH CDR1, VH CDR2, VH CDR3, VL CDR1, VLCDR2 and VL CDR3 amino acid sequence of R3-P3-A12. In one embodiment, the Sdc2 antibody is used for the treatment of a stroke in a subject, as provided herein. In one embodiment, the Sdc2 antibody is used for the treatment of ischemic stroke in a subject, as provided herein. In one embodiment, the Sdc2 antibody is used for the treatment of a neovascular eye disease in a subject, as provided herein. In one embodiment, the Sdc2 antibody is used for the treatment of AMD in a subject, as provided herein. In one embodiment, the Sdc2 antibody is used for the treatment of a cardiovascular disease in a subject, as provided herein. In one embodiment, the Sdc2 antibody is used for the treatment of acute myocardial infarction in a subject, as provided herein.

In one embodiment of the methods provided herein, the Sdc2 antibody is R4M-P3-A12. In one embodiment, the Sdc2 antibody is derived from R4M-P3-A12. In one embodiment, the Sdc2 antibody is an antibody fragment of R4M-P3-A12. In one embodiment, the Sdc2 antibody comprises a VH amino acid sequence of R4M-P3-A12. In one embodiment, the Sdc2 antibody comprises a VL amino acid sequence of R4M-P3-A12. In one embodiment, the Sdc2 antibody comprises a VH amino acid sequence and VL amino acid sequence of R4M-P3-A12. In one embodiment, the Sdc2 antibody comprises a VH CDR1, VH CDR2 and VH CDR3 amino acid sequence of R4M-P3-A12. In one embodiment, the Sdc2 antibody comprises a VL CDR1, VL CDR2 and VL CDR3 amino acid sequence of R4M-P3-A12. In one embodiment, the Sdc2 antibody comprises a VH CDR1, VH CDR2, VH CDR3, VL CDR1, VLCDR2 and VL CDR3 amino acid sequence of R4M-P3-A12. In one embodiment, the Sdc2 antibody is used for the treatment of a stroke in a subject, as provided herein. In one embodiment, the Sdc2 antibody is used for the treatment of ischemic stroke in a subject, as provided herein. In one embodiment, the Sdc2 antibody is used for the treatment of a neovascular eye disease in a subject, as provided herein. In one embodiment, the Sdc2 antibody is used for the treatment of AMD in a subject, as provided herein. In one embodiment, the Sdc2 antibody is used for the treatment of a cardiovascular disease in a subject, as provided herein. In one embodiment, the Sdc2 antibody is used for the treatment of acute myocardial infarction in a subject, as provided herein.

In one embodiment of the methods provided herein, the Sdc2 antibody is R4M-P1-A10. In one embodiment, the Sdc2 antibody is derived from R4M-P1-A10. In one embodiment, the Sdc2 antibody is an antibody fragment of R4M-P1-A10. In one embodiment, the Sdc2 antibody comprises a VH amino acid sequence of R4M-P1-A10. In one embodiment, the Sdc2 antibody comprises a VL amino acid sequence of R4M-P1-A10. In one embodiment, the Sdc2 antibody comprises a VH amino acid sequence and VL amino acid sequence of R4M-P1-A10. In one embodiment, the Sdc2 antibody comprises a VH CDR1, VH CDR2 and VH CDR3 amino acid sequence of R4M-P1-A10. In one embodiment, the Sdc2 antibody comprises a VL CDR1, VL CDR2 and VL CDR3 amino acid sequence of R4M-P1-A10. In one embodiment, the Sdc2 antibody comprises a VH CDR1, VH CDR2, VH CDR3, VL CDR1, VLCDR2 and VL CDR3 amino acid sequence of R4M-P1-A10. In one embodiment, the Sdc2 antibody is used for the treatment of a stroke in a subject, as provided herein. In one embodiment, the Sdc2 antibody is used for the treatment of ischemic stroke in a subject, as provided herein. In one embodiment, the Sdc2 antibody is used for the treatment of a neovascular eye disease in a subject, as provided herein. In one embodiment, the Sdc2 antibody is used for the treatment of AMD in a subject, as provided herein. In one embodiment, the Sdc2 antibody is used for the treatment of a cardiovascular disease in a subject, as provided herein. In one embodiment, the Sdc2 antibody is used for the treatment of acute myocardial infarction in a subject, as provided herein.

Exemplary binding agents that bind to Sdc2 are provided herein, for example in the Examples, as well as Tables 1, and 2-7.

Any of the Sdc2 antibodies provided herein are contemplated for use in any of the individual methods provided herein.

In some embodiments of the methods provided herein, the antibody that binds Sdc2 or the antigen binding fragment thereof comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:61; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:62. In some embodiments of the methods provided herein, the antibody that binds Sdc2 or the antigen binding fragment thereof comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 95; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:96. In some embodiments of the methods provided herein, the antibody that binds Sdc2 or the antigen binding fragment thereof comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 129; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 130. In some embodiments of the methods provided herein, the antibody that binds Sdc2 or the antigen binding fragment thereof comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 163; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 164. In some embodiments of the methods provided herein, the antibody that binds Sdc2 or the antigen binding fragment thereof comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 197; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 198. In some embodiments of the methods provided herein, the antibody that binds Sdc2 or the antigen binding fragment thereof comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:231; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:232. In some embodiments of the methods provided herein, the antibody that binds Sdc2 or the antigen binding fragment thereof comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 265; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:266. In some embodiments of the methods provided herein, the antibody that binds Sdc2 or the antigen binding fragment thereof comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:299; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:300. In some embodiments of the methods provided herein, the antibody that binds Sdc2 or the antigen binding fragment thereof comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:333; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:334. In some embodiments of the methods provided herein, the antibody that binds Sdc2 or the antigen binding fragment thereof comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:367; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 368. In some embodiments of the methods provided herein, the antibody that binds Sdc2 or the antigen binding fragment thereof comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:401; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:402. In some embodiments of the methods provided herein, the antibody that binds Sdc2 or the antigen binding fragment thereof comprises: (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 435; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:436.

In some embodiments of the methods provided herein, the antibody that binds Sdc2 or the antigen binding fragment thereof comprises: the VH CDR1, VH CDR2, VH CDR3, VL CDR1, VL CDR2, and VL CDR3 amino acid sequences are according to the Kabat numbering system; the VH CDR1, VH CDR2, VH CDR3, VL CDR1, VL CDR2, and VL CDR3 amino acid sequences are according to the Chothia numbering system; the VH CDR1, VH CDR2, VH CDR3, VL CDR1, VL CDR2, and VL CDR3 amino acid sequences are according to the AbM numbering system; the VH CDR1, VH CDR2, VH CDR3, VL CDR1, VL CDR2, and VL CDR3 amino acid sequences are according to the Contact numbering system; or the VH CDR1, VH CDR2, VH CDR3, VL CDR1, VL CDR2, and VL CDR3 amino acid sequences are according to the IMGT numbering system.

In some embodiments of the methods provided herein, the antibody that binds Sdc2 or the antigen binding fragment thereof comprises: (A) (i) a VH comprising a VH CDR1, a VH CDR2, and a VH CDR3 having an amino acid sequence of SEQ ID NOs: 31, 32 and 33, respectively; and (ii) a VL comprising a VL CDR1, a VL CDR2, and a VL CDR3 having an amino acid sequence of SEQ ID NOs: 34, 35 and 36, respectively; (B) (i) a VH comprising a VH CDR1, a VH CDR2, and a VH CDR3 having an amino acid sequence of SEQ ID NOs: 37, 38 and 39, respectively; and (ii) a VL comprising a VL CDR1, a VL CDR2, and a VL CDR3 having an amino acid sequence of SEQ ID NOs: 40, 41 and 42, respectively; (C) (i) a VH comprising a VH CDR1, a VH CDR2, and a VH CDR3 having an amino acid sequence of SEQ ID NOs: 43, 44 and 45, respectively; and (ii) a VL comprising a VL CDR1, a VL CDR2, and a VL CDR3 having an amino acid sequence of SEQ ID NOs: 46, 47 and 48, respectively; (D) (i) a VH comprising a VH CDR1, a VH CDR2, and a VH CDR3 having an amino acid sequence of SEQ ID NOs: 49, 50 and 51, respectively; and (ii) a VL comprising a VL CDR1, a VL CDR2, and a VL CDR3 having an amino acid sequence of SEQ ID NOs: 52, 53 and 54, respectively; or (E) (i) a VH comprising a VH CDR1, a VH CDR2, and a VH CDR3 having an amino acid sequence of SEQ ID NOs: 55, 56 and 57, respectively; and (ii) a VL comprising a VL CDR1, a VL CDR2, and a VL CDR3 having an amino acid sequence of SEQ ID NOs: 58, 59 and 60, respectively.

In some embodiments of the methods provided herein, the antibody that binds Sdc2 or the antigen binding fragment thereof comprises: (A) (i) a VH comprising a VH CDR1, a VH CDR2, and a VH CDR3 having an amino acid sequence of SEQ ID NOs: 65, 66 and 67, respectively; and (ii) a VL comprising a VL CDR1, a VL CDR2, and a VL CDR3 having an amino acid sequence of SEQ ID NOs: 68, 89 and 70, respectively; (B) (i) a VH comprising a VH CDR1, a VH CDR2, and a VH CDR3 having an amino acid sequence of SEQ ID NOs: 71, 72 and 73, respectively; and (ii) a VL comprising a VL CDR1, a VL CDR2, and a VL CDR3 having an amino acid sequence of SEQ ID NOs: 74, 75 and 76, respectively; (C) (i) a VH comprising a VH CDR1, a VH CDR2, and a VH CDR3 having an amino acid sequence of SEQ ID NOs: 77, 78 and 79, respectively; and (ii) a VL comprising a VL CDR1, a VL CDR2, and a VL CDR3 having an amino acid sequence of SEQ ID NOs: 80, 81 and 82, respectively; (D) (i) a VH comprising a VH CDR1, a VH CDR2, and a VH CDR3 having an amino acid sequence of SEQ ID NOs: 83, 84 and 85, respectively; and (ii) a VL comprising a VL CDR1, a VL CDR2, and a VL CDR3 having an amino acid sequence of SEQ ID NOs: 86, 87 and 88, respectively; or (E) (i) a VH comprising a VH CDR1, a VH CDR2, and a VH CDR3 having an amino acid sequence of SEQ ID NOs: 89, 90 and 91, respectively; and (ii) a VL comprising a VL CDR1, a VL CDR2, and a VL CDR3 having an amino acid sequence of SEQ ID NOs: 92, 93 and 94, respectively.

In some embodiments of the methods provided herein, the antibody that binds Sdc2 or the antigen binding fragment thereof comprises: (A) (i) a VH comprising a VH CDR1, a VH CDR2, and a VH CDR3 having an amino acid sequence of SEQ ID NOs: 99, 100 and 101, respectively; and (ii) a VL comprising a VL CDR1, a VL CDR2, and a VL CDR3 having an amino acid sequence of SEQ ID NOs: 102,103 and 104, respectively; (B) (i) a VH comprising a VH CDR1, a VH CDR2, and a VH CDR3 having an amino acid sequence of SEQ ID NOs: 105, 106 and 107, respectively; and (ii) a VL comprising a VL CDR1, a VL CDR2, and a VL CDR3 having an amino acid sequence of SEQ ID NOs: 108, 109 and 110, respectively; (C) (i) a VH comprising a VH CDR1, a VH CDR2, and a VH CDR3 having an amino acid sequence of SEQ ID NOs: 111, 112 and 113, respectively; and (ii) a VL comprising a VL CDR1, a VL CDR2, and a VL CDR3 having an amino acid sequence of SEQ ID NOs: 114, 115 and 116, respectively; (D) (i) a VH comprising a VH CDR1, a VH CDR2, and a VH CDR3 having an amino acid sequence of SEQ ID NOs: 117, 118 and 119, respectively; and (ii) a VL comprising a VL CDR1, a VL CDR2, and a VL CDR3 having an amino acid sequence of SEQ ID NOs: 120, 121 and 122, respectively; or (E) (i) a VH comprising a VH CDR1, a VH CDR2, and a VH CDR3 having an amino acid sequence of SEQ ID NOs: 123, 124 and 125, respectively; and (ii) a VL comprising a VL CDR1, a VL CDR2, and a VL CDR3 having an amino acid sequence of SEQ ID NOs: 126, 127 and 128, respectively.

In some embodiments of the methods provided herein, the antibody that binds Sdc2 or the antigen binding fragment thereof comprises: (A) (i) a VH comprising a VH CDR1, a VH CDR2, and a VH CDR3 having an amino acid sequence of SEQ ID NOs: 133, 134 and 135, respectively; and (ii) a VL comprising a VL CDR1, a VL CDR2, and a VL CDR3 having an amino acid sequence of SEQ ID NOs: 136, 137 and 138, respectively; (B) (i) a VH comprising a VH CDR1, a VH CDR2, and a VH CDR3 having an amino acid sequence of SEQ ID NOs: 139, 140 and 141, respectively; and (ii) a VL comprising a VL CDR1, a VL CDR2, and a VL CDR3 having an amino acid sequence of SEQ ID NOs: 142, 143 and 144, respectively; (C) (i) a VH comprising a VH CDR1, a VH CDR2, and a VH CDR3 having an amino acid sequence of SEQ ID NOs: 145, 146 and 147, respectively; and (ii) a VL comprising a VL CDR1, a VL CDR2, and a VL CDR3 having an amino acid sequence of SEQ ID NOs: 148, 149 and 150, respectively; (D) (i) a VH comprising a VH CDR1, a VH CDR2, and a VH CDR3 having an amino acid sequence of SEQ ID NOs: 151, 152 and 153, respectively; and (ii) a VL comprising a VL CDR1, a VL CDR2, and a VL CDR3 having an amino acid sequence of SEQ ID NOs: 154, 155 and 156, respectively; or (E) (i) a VH comprising a VH CDR1, a VH CDR2, and a VH CDR3 having an amino acid sequence of SEQ ID NOs: 157, 158 and 159, respectively; and (ii) a VL comprising a VL CDR1, a VL CDR2, and a VL CDR3 having an amino acid sequence of SEQ ID NOs: 160, 161 and 162, respectively.

In some embodiments of the methods provided herein, the antibody that binds Sdc2 or the antigen binding fragment thereof comprises: (A) (i) a VH comprising a VH CDR1, a VH CDR2, and a VH CDR3 having an amino acid sequence of SEQ ID NOs: 167, 168 and 169, respectively; and (ii) a VL comprising a VL CDR1, a VL CDR2, and a VL CDR3 having an amino acid sequence of SEQ ID NOs: 170, 171 and 172, respectively; (B) (i) a VH comprising a VH CDR1, a VH CDR2, and a VH CDR3 having an amino acid sequence of SEQ ID NOs: 173, 174 and 175, respectively; and (ii) a VL comprising a VL CDR1, a VL CDR2, and a VL CDR3 having an amino acid sequence of SEQ ID NOs: 176, 177 and 178, respectively; (C) (i) a VH comprising a VH CDR1, a VH CDR2, and a VH CDR3 having an amino acid sequence of SEQ ID NOs: 179, 180 and 181, respectively; and (ii) a VL comprising a VL CDR1, a VL CDR2, and a VL CDR3 having an amino acid sequence of SEQ ID NOs: 182, 183 and 184, respectively; (D) (i) a VH comprising a VH CDR1, a VH CDR2, and a VH CDR3 having an amino acid sequence of SEQ ID NOs: 185, 186 and 187, respectively; and (ii) a VL comprising a VL CDR1, a VL CDR2, and a VL CDR3 having an amino acid sequence of SEQ ID NOs: 188, 189 and 190, respectively; or (E) (i) a VH comprising a VH CDR1, a VH CDR2, and a VH CDR3 having an amino acid sequence of SEQ ID NOs: 191, 192 and 193, respectively; and (ii) a VL comprising a VL CDR1, a VL CDR2, and a VL CDR3 having an amino acid sequence of SEQ ID NOs: 194, 195 and 196, respectively.

In some embodiments of the methods provided herein, the antibody that binds Sdc2 or the antigen binding fragment thereof comprises: (A) (i) a VH comprising a VH CDR1, a VH CDR2, and a VH CDR3 having an amino acid sequence of SEQ ID NOs: 201, 202 and 203, respectively; and (ii) a VL comprising a VL CDR1, a VL CDR2, and a VL CDR3 having an amino acid sequence of SEQ ID NOs: 204, 205 and 206, respectively; (B) (i) a VH comprising a VH CDR1, a VH CDR2, and a VH CDR3 having an amino acid sequence of SEQ ID NOs: 207, 208 and 209, respectively; and (ii) a VL comprising a VL CDR1, a VL CDR2, and a VL CDR3 having an amino acid sequence of SEQ ID NOs: 210, 211 and 212, respectively; (C) (i) a VH comprising a VH CDR1, a VH CDR2, and a VH CDR3 having an amino acid sequence of SEQ ID NOs: 213, 214 and 215, respectively; and (ii) a VL comprising a VL CDR1, a VL CDR2, and a VL CDR3 having an amino acid sequence of SEQ ID NOs: 216, 217 and 218, respectively; (D) (i) a VH comprising a VH CDR1, a VH CDR2, and a VH CDR3 having an amino acid sequence of SEQ ID NOs: 219, 220 and 221, respectively; and (ii) a VL comprising a VL CDR1, a VL CDR2, and a VL CDR3 having an amino acid sequence of SEQ ID NOs: 222, 223 and 224, respectively; or (E) (i) a VH comprising a VH CDR1, a VH CDR2, and a VH CDR3 having an amino acid sequence of SEQ ID NOs: 225, 226 and 227, respectively; and (ii) a VL comprising a VL CDR1, a VL CDR2, and a VL CDR3 having an amino acid sequence of SEQ ID NOs: 228, 229 and 230, respectively.

In some embodiments of the methods provided herein, the antibody that binds Sdc2 or the antigen binding fragment thereof comprises: (A) (i) a VH comprising a VH CDR1, a VH CDR2, and a VH CDR3 having an amino acid sequence of SEQ ID NOs: 235, 236 and 237, respectively; and (ii) a VL comprising a VL CDR1, a VL CDR2, and a VL CDR3 having an amino acid sequence of SEQ ID NOs: 238, 239 and 240, respectively; (B) (i) a VH comprising a VH CDR1, a VH CDR2, and a VH CDR3 having an amino acid sequence of SEQ ID NOs: 241, 242 and 243, respectively; and (ii) a VL comprising a VL CDR1, a VL CDR2, and a VL CDR3 having an amino acid sequence of SEQ ID NOs: 244, 245 and 246, respectively; (C) (i) a VH comprising a VH CDR1, a VH CDR2, and a VH CDR3 having an amino acid sequence of SEQ ID NOs: 247, 248 and 249, respectively; and (ii) a VL comprising a VL CDR1, a VL CDR2, and a VL CDR3 having an amino acid sequence of SEQ ID NOs: 250, 251 and 252, respectively; (D) (i) a VH comprising a VH CDR1, a VH CDR2, and a VH CDR3 having an amino acid sequence of SEQ ID NOs: 253, 254 and 255, respectively; and (ii) a VL comprising a VL CDR1, a VL CDR2, and a VL CDR3 having an amino acid sequence of SEQ ID NOs: 256, 257 and 258, respectively; or (E) (i) a VH comprising a VH CDR1, a VH CDR2, and a VH CDR3 having an amino acid sequence of SEQ ID NOs: 259, 260 and 261, respectively; and (ii) a VL comprising a VL CDR1, a VL CDR2, and a VL CDR3 having an amino acid sequence of SEQ ID NOs: 262, 263 and 264, respectively.

In some embodiments of the methods provided herein, the antibody that binds Sdc2 or the antigen binding fragment thereof comprises: (A) (i) a VH comprising a VH CDR1, a VH CDR2, and a VH CDR3 having an amino acid sequence of SEQ ID NOs: 269, 270 and 271, respectively; and (ii) a VL comprising a VL CDR1, a VL CDR2, and a VL CDR3 having an amino acid sequence of SEQ ID NOs: 272, 273 and 274, respectively; (B) (i) a VH comprising a VH CDR1, a VH CDR2, and a VH CDR3 having an amino acid sequence of SEQ ID NOs: 275, 276 and 277, respectively; and (ii) a VL comprising a VL CDR1, a VL CDR2, and a VL CDR3 having an amino acid sequence of SEQ ID NOs: 278, 279 and 280, respectively; (C) (i) a VH comprising a VH CDR1, a VH CDR2, and a VH CDR3 having an amino acid sequence of SEQ ID NOs: 281, 282 and 283, respectively; and (ii) a VL comprising a VL CDR1, a VL CDR2, and a VL CDR3 having an amino acid sequence of SEQ ID NOs: 284, 285 and 286, respectively; (D) (i) a VH comprising a VH CDR1, a VH CDR2, and a VH CDR3 having an amino acid sequence of SEQ ID NOs: 287, 288 and 289, respectively; and (ii) a VL comprising a VL CDR1, a VL CDR2, and a VL CDR3 having an amino acid sequence of SEQ ID NOs: 290, 292 and 292, respectively; or (E) (i) a VH comprising a VH CDR1, a VH CDR2, and a VH CDR3 having an amino acid sequence of SEQ ID NOs: 293, 294 and 295, respectively; and (ii) a VL comprising a VL CDR1, a VL CDR2, and a VL CDR3 having an amino acid sequence of SEQ ID NOs: 296, 297 and 298, respectively.

In some embodiments of the methods provided herein, the antibody that binds Sdc2 or the antigen binding fragment thereof comprises: (A) (i) a VH comprising a VH CDR1, a VH CDR2, and a VH CDR3 having an amino acid sequence of SEQ ID NOs: 303, 304 and 305, respectively; and (ii) a VL comprising a VL CDR1, a VL CDR2, and a VL CDR3 having an amino acid sequence of SEQ ID NOs: 306, 307 and 308, respectively; (B) (i) a VH comprising a VH CDR1, a VH CDR2, and a VH CDR3 having an amino acid sequence of SEQ ID NOs: 309, 310 and 311, respectively; and (ii) a VL comprising a VL CDR1, a VL CDR2, and a VL CDR3 having an amino acid sequence of SEQ ID NOs: 312, 313 and 314, respectively; (C) (i) a VH comprising a VH CDR1, a VH CDR2, and a VH CDR3 having an amino acid sequence of SEQ ID NOs: 315, 316 and 317, respectively; and (ii) a VL comprising a VL CDR1, a VL CDR2, and a VL CDR3 having an amino acid sequence of SEQ ID NOs: 318, 319 and 320, respectively; (D) (i) a VH comprising a VH CDR1, a VH CDR2, and a VH CDR3 having an amino acid sequence of SEQ ID NOs: 321, 322 and 323, respectively; and (ii) a VL comprising a VL CDR1, a VL CDR2, and a VL CDR3 having an amino acid sequence of SEQ ID NOs: 324, 325 and 326, respectively; or (E) (i) a VH comprising a VH CDR1, a VH CDR2, and a VH CDR3 having an amino acid sequence of SEQ ID NOs: 327, 328 and 329, respectively; and (ii) a VL comprising a VL CDR1, a VL CDR2, and a VL CDR3 having an amino acid sequence of SEQ ID NOs: 330, 331 and 332, respectively.

In some embodiments of the methods provided herein, the antibody that binds Sdc2 or the antigen binding fragment thereof comprises: (A) (i) a VH comprising a VH CDR1, a VH CDR2, and a VH CDR3 having an amino acid sequence of SEQ ID NOs: 337, 338 and 339, respectively; and (ii) a VL comprising a VL CDR1, a VL CDR2, and a VL CDR3 having an amino acid sequence of SEQ ID NOs: 340, 341 and 342, respectively; (B) (i) a VH comprising a VH CDR1, a VH CDR2, and a VH CDR3 having an amino acid sequence of SEQ ID NOs: 343, 344 and 345, respectively; and (ii) a VL comprising a VL CDR1, a VL CDR2, and a VL CDR3 having an amino acid sequence of SEQ ID NOs: 346, 347 and 348, respectively; (C) (i) a VH comprising a VH CDR1, a VH CDR2, and a VH CDR3 having an amino acid sequence of SEQ ID NOs: 349, 350 and 351, respectively; and (ii) a VL comprising a VL CDR1, a VL CDR2, and a VL CDR3 having an amino acid sequence of SEQ ID NOs: 352, 353 and 354, respectively; (D) (i) a VH comprising a VH CDR1, a VH CDR2, and a VH CDR3 having an amino acid sequence of SEQ ID NOs: 355, 356 and 357, respectively; and (ii) a VL comprising a VL CDR1, a VL CDR2, and a VL CDR3 having an amino acid sequence of SEQ ID NOs: 358, 359 and 360, respectively; or (E) (i) a VH comprising a VH CDR1, a VH CDR2, and a VH CDR3 having an amino acid sequence of SEQ ID NOs: 361, 362 and 363, respectively; and (ii) a VL comprising a VL CDR1, a VL CDR2, and a VL CDR3 having an amino acid sequence of SEQ ID NOs: 364, 365 and 366, respectively.

In some embodiments of the methods provided herein, the antibody that binds Sdc2 or the antigen binding fragment thereof comprises: (A) (i) a VH comprising a VH CDR1, a VH CDR2, and a VH CDR3 having an amino acid sequence of SEQ ID NOs: 371, 372 and 373, respectively; and (ii) a VL comprising a VL CDR1, a VL CDR2, and a VL CDR3 having an amino acid sequence of SEQ ID NOs: 374, 375 and 376, respectively; (B) (i) a VH comprising a VH CDR1, a VH CDR2, and a VH CDR3 having an amino acid sequence of SEQ ID NOs: 377, 378 and 379, respectively; and (ii) a VL comprising a VL CDR1, a VL CDR2, and a VL CDR3 having an amino acid sequence of SEQ ID NOs: 380, 381 and 382, respectively; (C) (i) a VH comprising a VH CDR1, a VH CDR2, and a VH CDR3 having an amino acid sequence of SEQ ID NOs: 383, 384 and 385, respectively; and (ii) a VL comprising a VL CDR1, a VL CDR2, and a VL CDR3 having an amino acid sequence of SEQ ID NOs: 386, 387 and 388, respectively; (D) (i) a VH comprising a VH CDR1, a VH CDR2, and a VH CDR3 having an amino acid sequence of SEQ ID NOs: 389, 390 and 391, respectively; and (ii) a VL comprising a VL CDR1, a VL CDR2, and a VL CDR3 having an amino acid sequence of SEQ ID NOs: 392, 393 and 394, respectively; or (E) (i) a VH comprising a VH CDR1, a VH CDR2, and a VH CDR3 having an amino acid sequence of SEQ ID NOs: 395, 396 and 397, respectively; and (ii) a VL comprising a VL CDR1, a VL CDR2, and a VL CDR3 having an amino acid sequence of SEQ ID NOs: 398, 399 and 400, respectively. In some embodiments of the methods provided herein, the antibody that binds Sdc2 or the antigen binding fragment thereof comprises: (A) (i) a VH comprising a VH CDR1, a VH CDR2, and a VH CDR3 having an amino acid sequence of SEQ ID NOs: 405, 406 and 407, respectively; and (ii) a VL comprising a VL CDR1, a VL CDR2, and a VL CDR3 having an amino acid sequence of SEQ ID NOs: 408, 409 and 410, respectively; (B) (i) a VH comprising a VH CDR1, a VH CDR2, and a VH CDR3 having an amino acid sequence of SEQ ID NOs: 411, 412 and 413, respectively; and (ii) a VL comprising a VL CDR1, a VL CDR2, and a VL CDR3 having an amino acid sequence of SEQ ID NOs: 414, 415 and 416, respectively; (C) (i) a VH comprising a VH CDR1, a VH CDR2, and a VH CDR3 having an amino acid sequence of SEQ ID NOs: 417, 418 and 419, respectively; and (ii) a VL comprising a VL CDR1, a VL CDR2, and a VL CDR3 having an amino acid sequence of SEQ ID NOs: 420, 421 and 422, respectively; (D) (i) a VH comprising a VH CDR1, a VH CDR2, and a VH CDR3 having an amino acid sequence of SEQ ID NOs: 423, 424 and 425, respectively; and (ii) a VL comprising a VL CDR1, a VL CDR2, and a VL CDR3 having an amino acid sequence of SEQ ID NOs: 426, 427 and 428, respectively; or (E) (i) a VH comprising a VH CDR1, a VH CDR2, and a VH CDR3 having an amino acid sequence of SEQ ID NOs: 429, 430 and 431, respectively; and (ii) a VL comprising a VL CDR1, a VL CDR2, and a VL CDR3 having an amino acid sequence of SEQ ID NOs: 432, 433 and 434, respectively.

In some embodiments of the methods provided herein, the antibody that binds Sdc2 or the antigen binding fragment thereof comprises: (i) a VH having an amino acid sequence having at least 95% identity to an amino acid sequence of SEQ ID NO:61; and (ii) a VL having an amino acid sequence having at least 95% identity to an amino acid sequence of SEQ ID NO:62. In some embodiments of the methods provided herein, the antibody that binds Sdc2 or the antigen binding fragment thereof comprises: (i) a VH having an amino acid sequence having at least 95% identity to an amino acid sequence of SEQ ID NO:95; and (ii) a VL having an amino acid sequence having at least 95% identity to an amino acid sequence of SEQ ID NO:96. In some embodiments of the methods provided herein, the antibody that binds Sdc2 or the antigen binding fragment thereof comprises: (i) a VH having an amino acid sequence having at least 95% identity to an amino acid sequence of SEQ ID NO:129; and (ii) a VL having an amino acid sequence having at least 95% identity to an amino acid sequence of SEQ ID NO: 130. In some embodiments of the methods provided herein, the antibody that binds Sdc2 or the antigen binding fragment thereof comprises: (i) a VH having an amino acid sequence having at least 95% identity to an amino acid sequence of SEQ ID NO: 163; and (ii) a VL having an amino acid sequence having at least 95% identity to an amino acid sequence of SEQ ID NO: 164. In some embodiments of the methods provided herein, the antibody that binds Sdc2 or the antigen binding fragment thereof comprises: (i) a VH having an amino acid sequence having at least 95% identity to an amino acid sequence of SEQ ID NO: 197; and (ii) a VL having an amino acid sequence having at least 95% identity to an amino acid sequence of SEQ ID NO: 198. In some embodiments of the methods provided herein, the antibody that binds Sdc2 or the antigen binding fragment thereof comprises: (i) a VH having an amino acid sequence having at least 95% identity to an amino acid sequence of SEQ ID NO:231; and (ii) a VL having an amino acid sequence having at least 95% identity to an amino acid sequence of SEQ ID NO:232. In some embodiments of the methods provided herein, the antibody that binds Sdc2 or the antigen binding fragment thereof comprises: (i) a VH having an amino acid sequence having at least 95% identity to an amino acid sequence of SEQ ID NO: 265; and (ii) a VL having an amino acid sequence having at least 95% identity to an amino acid sequence of SEQ ID NO:266. In some embodiments of the methods provided herein, the antibody that binds Sdc2 or the antigen binding fragment thereof comprises: (i) a VH having an amino acid sequence having at least 95% identity to an amino acid sequence of SEQ ID NO:299; and (ii) a VL having an amino acid sequence having at least 95% identity to an amino acid sequence of SEQ ID NO:300. In some embodiments of the methods provided herein, the antibody that binds Sdc2 or the antigen binding fragment thereof comprises: (i) a VH having an amino acid sequence having at least 95% identity to an amino acid sequence of SEQ ID NO:333; and (ii) a VL having an amino acid sequence having at least 95% identity to an amino acid sequence of SEQ ID NO:334. In some embodiments of the methods provided herein, the antibody that binds Sdc2 or the antigen binding fragment thereof comprises: (i) a VH having an amino acid sequence having at least 95% identity to an amino acid sequence of SEQ ID NO: 367; and (ii) a VL having an amino acid sequence having at least 95% identity to an amino acid sequence of SEQ ID NO:368. In some embodiments of the methods provided herein, the antibody that binds Sdc2 or the antigen binding fragment thereof comprises: (i) a VH having an amino acid sequence having at least 95% identity to an amino acid sequence of SEQ ID NO:401; and (ii) a VL having an amino acid sequence having at least 95% identity to an amino acid sequence of SEQ ID NO:402. In some embodiments of the methods provided herein, the antibody that binds Sdc2 or the antigen binding fragment thereof comprises: (i) a VH having an amino acid sequence having at least 95% identity to an amino acid sequence of SEQ ID NO:435; and (ii) a VL having an amino acid sequence having at least 95% identity to an amino acid sequence of SEQ ID NO:436.

In some embodiments of the methods provided herein, the antibody that binds Sdc2 or the antigen binding fragment thereof comprises: (i) a VH having an amino acid sequence SEQ ID NO:61; and (ii) a VL having an amino acid sequence SEQ ID NO:62. In some embodiments of the methods provided herein, the antibody that binds Sdc2 or the antigen binding fragment thereof comprises: (i) a VH having an amino acid sequence SEQ ID NO:95; and (ii) a VL having an amino acid sequence SEQ ID NO:96. In some embodiments of the methods provided herein, the antibody that binds Sdc2 or the antigen binding fragment thereof comprises: (i) a VH having an amino acid sequence SEQ ID NO:129; and (ii) a VL having an amino acid sequence SEQ ID NO:130. In some embodiments of the methods provided herein, the antibody that binds Sdc2 or the antigen binding fragment thereof comprises: (i) a VH having an amino acid sequence SEQ ID NO: 163; and (ii) a VL having an amino acid sequence SEQ ID NO: 164. In some embodiments of the methods provided herein, the antibody that binds Sdc2 or the antigen binding fragment thereof comprises: (i) a VH having an amino acid sequence SEQ ID NO:197; and (ii) a VL having an amino acid sequence SEQ ID NO:198. In some embodiments of the methods provided herein, the antibody that binds Sdc2 or the antigen binding fragment thereof comprises: (i) a VH having an amino acid sequence SEQ ID NO:231; and (ii) a VL having an amino acid sequence SEQ ID NO:232. In some embodiments of the methods provided herein, the antibody that binds Sdc2 or the antigen binding fragment thereof comprises: (i) a VH having an amino acid sequence SEQ ID NO:265; and (ii) a VL having an amino acid sequence SEQ ID NO:266. In some embodiments of the methods provided herein, the antibody that binds Sdc2 or the antigen binding fragment thereof comprises: (i) a VH having an amino acid sequence SEQ ID NO:299; and (ii) a VL having an amino acid sequence SEQ ID NO:300. In some embodiments of the methods provided herein, the antibody that binds Sdc2 or the antigen binding fragment thereof comprises: (i) a VH having an amino acid sequence SEQ ID NO:333; and (ii) a VL having an amino acid sequence SEQ ID NO:334. In some embodiments of the methods provided herein, the antibody that binds Sdc2 or the antigen binding fragment thereof comprises: (i) a VH having an amino acid sequence SEQ ID NO:367; and (ii) a VL having an amino acid sequence SEQ ID NO:368. In some embodiments of the methods provided herein, the antibody that binds Sdc2 or the antigen binding fragment thereof comprises: (i) a VH having an amino acid sequence SEQ ID NO:401; and (ii) a VL having an amino acid sequence SEQ ID NO:402. In some embodiments of the methods provided herein, the antibody that binds Sdc2 or the antigen binding fragment thereof comprises: (i) a VH having an amino acid sequence SEQ ID NO:435; and (ii) a VL having an amino acid sequence SEQ ID NO:436.

In some embodiments of the methods provided herein, the antibody that binds Sdc2 or the antigen binding fragment thereof comprises: (i) a heavy chain having an amino acid sequence having at least 95% identity to an amino acid sequence of SEQ ID NO:63; and (ii) a light chain having an amino acid sequence having at least 95% identity to an amino acid sequence of SEQ ID NO:64. In some embodiments of the methods provided herein, the antibody that binds Sdc2 or the antigen binding fragment thereof comprises: (i) a heavy chain having an amino acid sequence having at least 95% identity to an amino acid sequence of SEQ ID NO:97; and (ii) a light chain having an amino acid sequence having at least 95% identity to an amino acid sequence of SEQ ID NO:98. In some embodiments of the methods provided herein, the antibody that binds Sdc2 or the antigen binding fragment thereof comprises: (i) a heavy chain having an amino acid sequence having at least 95% identity to an amino acid sequence of SEQ ID NO:131; and (ii) a light chain having an amino acid sequence having at least 95% identity to an amino acid sequence of SEQ ID NO: 132. In some embodiments of the methods provided herein, the antibody that binds Sdc2 or the antigen binding fragment thereof comprises: (i) a heavy chain having an amino acid sequence having at least 95% identity to an amino acid sequence of SEQ ID NO: 165; and (ii) a light chain having an amino acid sequence having at least 95% identity to an amino acid sequence of SEQ ID NO:166. In some embodiments of the methods provided herein, the antibody that binds Sdc2 or the antigen binding fragment thereof comprises: (i) a heavy chain having an amino acid sequence having at least 95% identity to an amino acid sequence of SEQ ID NO:199; and (ii) a light chain having an amino acid sequence having at least 95% identity to an amino acid sequence of SEQ ID NO:200. In some embodiments of the methods provided herein, the antibody that binds Sdc2 or the antigen binding fragment thereof comprises: (i) a heavy chain having an amino acid sequence having at least 95% identity to an amino acid sequence of SEQ ID NO:233; and (ii) a light chain having an amino acid sequence having at least 95% identity to an amino acid sequence of SEQ ID NO:234. In some embodiments of the methods provided herein, the antibody that binds Sdc2 or the antigen binding fragment thereof comprises: (i) a heavy chain having an amino acid sequence having at least 95% identity to an amino acid sequence of SEQ ID NO:267; and (ii) a light chain having an amino acid sequence having at least 95% identity to an amino acid sequence of SEQ ID NO:268. In some embodiments of the methods provided herein, the antibody that binds Sdc2 or the antigen binding fragment thereof comprises: (i) a heavy chain having an amino acid sequence having at least 95% identity to an amino acid sequence of SEQ ID NO:301; and (ii) a light chain having an amino acid sequence having at least 95% identity to an amino acid sequence of SEQ ID NO:302. In some embodiments of the methods provided herein, the antibody that binds Sdc2 or the antigen binding fragment thereof comprises: (i) a heavy chain having an amino acid sequence having at least 95% identity to an amino acid sequence of SEQ ID NO:335; and (ii) a light chain having an amino acid sequence having at least 95% identity to an amino acid sequence of SEQ ID NO:336. In some embodiments of the methods provided herein, the antibody that binds Sdc2 or the antigen binding fragment thereof comprises: (i) a heavy chain having an amino acid sequence having at least 95% identity to an amino acid sequence of SEQ ID NO:369; and (ii) a light chain having an amino acid sequence having at least 95% identity to an amino acid sequence of SEQ ID NO:370. In some embodiments of the methods provided herein, the antibody that binds Sdc2 or the antigen binding fragment thereof comprises: (i) a heavy chain having an amino acid sequence having at least 95% identity to an amino acid sequence of SEQ ID NO:403; and (ii) a light chain having an amino acid sequence having at least 95% identity to an amino acid sequence of SEQ ID NO:404. In some embodiments of the methods provided herein, the antibody that binds Sdc2 or the antigen binding fragment thereof comprises: (i) a heavy chain having an amino acid sequence having at least 95% identity to an amino acid sequence of SEQ ID NO:437; and (ii) a light chain having an amino acid sequence having at least 95% identity to an amino acid sequence of SEQ ID NO:438.

In some embodiments of the methods provided herein, the antibody that binds Sdc2 or the antigen binding fragment thereof comprises: (i) a heavy chain having an amino acid sequence SEQ ID NO:63; and (ii) a light chain having an amino acid sequence SEQ ID NO:64. In some embodiments of the methods provided herein, the antibody that binds Sdc2 or the antigen binding fragment thereof comprises: (i) a heavy chain having an amino acid sequence SEQ ID NO:97; and (ii) a light chain having an amino acid sequence SEQ ID NO:98. In some embodiments of the methods provided herein, the antibody that binds Sdc2 or the antigen binding fragment thereof comprises: (i) a heavy chain having an amino acid sequence SEQ ID NO: 131; and (ii) a light chain having an amino acid sequence SEQ ID NO:132. In some embodiments of the methods provided herein, the antibody that binds Sdc2 or the antigen binding fragment thereof comprises: (i) a heavy chain having an amino acid sequence SEQ ID NO: 165; and (ii) a light chain having an amino acid sequence SEQ ID NO:166. In some embodiments of the methods provided herein, the antibody that binds Sdc2 or the antigen binding fragment thereof comprises: (i) a heavy chain having an amino acid sequence SEQ ID NO: 199; and (ii) a light chain having an amino acid sequence SEQ ID NO:200. In some embodiments of the methods provided herein, the antibody that binds Sdc2 or the antigen binding fragment thereof comprises: (i) a heavy chain having an amino acid sequence SEQ ID NO:233; and (ii) a light chain having an amino acid sequence SEQ ID NO:234. In some embodiments of the methods provided herein, the antibody that binds Sdc2 or the antigen binding fragment thereof comprises: (i) a heavy chain having an amino acid sequence SEQ ID NO:267; and (ii) a light chain having an amino acid sequence SEQ ID NO:268. In some embodiments of the methods provided herein, the antibody that binds Sdc2 or the antigen binding fragment thereof comprises: (i) a heavy chain having an amino acid sequence SEQ ID NO:301; and (ii) a light chain having an amino acid sequence SEQ ID NO:302. In some embodiments of the methods provided herein, the antibody that binds Sdc2 or the antigen binding fragment thereof comprises: (i) a heavy chain having an amino acid sequence SEQ ID NO:335; and (ii) a light chain having an amino acid sequence SEQ ID NO:336. In some embodiments of the methods provided herein, the antibody that binds Sdc2 or the antigen binding fragment thereof comprises: (i) a heavy chain having an amino acid sequence SEQ ID NO:369; and (ii) a light chain having an amino acid sequence SEQ ID NO:370. In some embodiments of the methods provided herein, the antibody that binds Sdc2 or the antigen binding fragment thereof comprises: (i) a heavy chain having an amino acid sequence SEQ ID NO:403; and (ii) a light chain having an amino acid sequence SEQ ID NO:404. In some embodiments of the methods provided herein, the antibody that binds Sdc2 or the antigen binding fragment thereof comprises: (i) a heavy chain having an amino acid sequence SEQ ID NO:437; and (ii) a light chain having an amino acid sequence SEQ ID NO:438.

In some embodiments of the methods provided herein, the antibody is a humanized antibody or a fully human antibody. In some embodiments, the antibody is an IgG antibody. In some embodiments, the IgG antibody is an IgG1, IgG2, IgG3, or IgG4 antibody. In some embodiments, the antibody comprises a kappa light chain. In some embodiments, the antibody comprises a lambda light chain. In some embodiments, the antibody is a monoclonal antibody. Any of the Sdc2 antibodies provided herein are contemplated for use in any of the individual methods provided herein.

### 6.8 Pharmaceutical Compositions, Dosage Forms, and Administration

In another aspect, provided is a pharmaceutical composition comprising an anti-Sdc2 antibody provided herein, and a pharmaceutically acceptable carrier. In one aspect, provided is a method of producing the pharmaceutical composition, comprising combining the anti-Sdc2 antibody with a pharmaceutically acceptable carrier to obtain the pharmaceutical composition.

In another general aspect, provided is a pharmaceutical composition comprising an anti-Sdc2 antibody provided herein and a pharmaceutically acceptable carrier. In certain embodiments, the antibody is isolated. Also provided is a method of producing the pharmaceutical composition, comprising combining the antibody with a pharmaceutically acceptable carrier to obtain the pharmaceutical composition. Any of the antibodies provided herein are contemplated in the pharmaceutical compositions.

The term “pharmaceutical composition” as used herein means a product comprising an antibody provided herein together with a pharmaceutically acceptable carrier. Antibodies of provided herein and compositions comprising them are also useful in the manufacture of a medicament for therapeutic applications.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. Pharmaceutically acceptable carriers are a non-toxic material that does not interfere with the effectiveness of a composition provided herein the biological activity of a composition provided herein. According to particular embodiments, in view of the present disclosure, any pharmaceutically acceptable carrier suitable for use in an antibody pharmaceutical composition can be used herein.

The formulation of pharmaceutically active ingredients with pharmaceutically acceptable carriers is known in the art, e.g., Remington: The Science and Practice of Pharmacy (e.g., 21st edition (2005), and any later editions). Non-limiting examples of additional ingredients include buffers, diluents, solvents, tonicity regulating agents, preservatives, stabilizers, and chelating agents. One or more pharmaceutically acceptable carriers can be used in formulating the pharmaceutical compositions provided herein.

Pharmaceutical compositions comprising an antibody are prepared for storage by mixing the antibody having the desired degree of purity with optional physiologically acceptable carriers, excipients, or stabilizers (see, e.g., Remington, Remington's Pharmaceutical Sciences (18th ed. 1980)) in the form of aqueous solutions or lyophilized or other dried forms.

The antibodies of the present disclosure may be formulated in any suitable form for delivery to a target cell/tissue, e.g., as microcapsules or macroemulsions (Remington, supra; Park et al., 2005, Molecules 10:146-61; Malik et al., 2007, Curr. Drug. Deliv. 4:141-51), as sustained release formulations (Putney and Burke, 1998, Nature Biotechnol. 16:153-57), or in liposomes (Maclean et al., 1997, Int. J. Oncol. 11:325-32; Kontermann, 2006, Curr. Opin. Mol. Ther. 8:39-45).

An antibody provided herein can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nanoparticles, and nanocapsules) or in macroemulsions. Such techniques are disclosed, for example, in Remington, supra.

Various compositions and delivery systems are known and can be used with an antibody that binds to Sdc2 as described herein, including, but not limited to, encapsulation in liposomes, microparticles, microcapsules, recombinant cells capable of expressing the antibody, receptor-mediated endocytosis (see, e.g., Wu and Wu, 1987, J. Biol. Chem. 262:4429-32), construction of a nucleic acid as part of a retroviral or other vector, etc. In another embodiment, a composition can be provided as a controlled release or sustained release system. In one embodiment, a pump may be used to achieve controlled or sustained release (see, e.g., Langer, supra; Sefton, 1987, Crit. Ref. Biomed. Eng. 14:201-40; Buchwald et al., 1980, Surgery 88:507-16; and Saudek et al., 1989, N. Engl. J. Med. 321:569-74). In another embodiment, polymeric materials can be used to achieve controlled or sustained release of a prophylactic or therapeutic agent (e.g., an antibody that binds to Sdc2 as described herein) or a composition provided herein (see, e.g., Medical Applications of Controlled Release (Langer and Wise eds., 1974); Controlled Drug Bioavailability, Drug Product Design and Performance (Smolen and Ball eds., 1984); Ranger and Peppas, 1983, J. Macromol. Sci. Rev. Macromol. Chem. 23:61-126; Levy et al., 1985, Science 228:190-92; During et al., 1989, Ann. Neurol. 25:351-56; Howard et al., 1989, J. Neurosurg. 71:105-12; U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable.

In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, 1990, Science 249:1527-33. Any technique known to one of skill in the art can be used to produce sustained release formulations comprising one or more antibodies that bind to Sdc2 as described herein (see, e.g., U.S. Pat. No. 4,526,938, PCT publication Nos. WO 91/05548 and WO 96/20698, Ning et al., 1996, Radiotherapy & Oncology 39:179-89; Song et al., 1995, PDA J. of Pharma. Sci. & Tech. 50:372-97; Cleek et al., 1997, Pro. Int'l. Symp. Control. Rel. Bioact. Mater. 24:853-54; and Lam et al., 1997, Proc. Int'l. Symp. Control Rel. Bioact. Mater. 24:759-60).

Appropriate pharmaceutical dosage forms are discussed herein. The regimen of administration may affect what constitutes an effective amount. The therapeutic formulations may be administered to the subject either prior to or after the onset of ARDS. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions comprising the syndecan-2 antibody or antigen-binding fragment thereof to a subject, preferably a mammal, more preferably a human, may be carried out using known procedures, at dosages and for periods of time effective to treat ARDS in the subject. An effective amount of the therapeutic compound necessary to achieve a therapeutic effect may vary according to factors such as the state of the disease or disorder in the subject; the age, sex, and weight of the subject; and the ability of the therapeutic compound to treat a disease in the subject. Dosage regimens may be adjusted to provide the optimum therapeutic response. For example, several divided doses may be administered daily, or the dose may be proportionally reduced as indicated by the exigencies of the therapeutic situation. In another non-limiting example, an effective dose of the therapeutic compound is administered as a single-dose within a certain time of an acute injury event such as a myocardial infarction or stroke A non-limiting example of an effective dose range for a composition comprising a syndecan-2 antibody or antigen-binding fragment thereof is from about 0.5 and 5,000 mg/kg of body weight/per day. One of ordinary skill in the art would be able to study the relevant factors and make the determination regarding the effective amount of the composition comprising a syndecan-2 disrupting agent without undue experimentation. For example, an effective dose in humans could be established via the measurement or determination of a biomarker present in a bodily fluid, such as blood, that correlates with vascular leak in vivo.

Actual dosage levels of the active ingredients in the pharmaceutical composition comprising a syndecan-2 antibody or antigen-binding fragment thereof may be varied so as to obtain an amount of the active ingredient (including but not limited to the syndecan-2 antibody or antigen-binding fragment thereof) that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a patient.

In certain embodiments, the compositions comprising the syndecan-2 antibody or antigen-binding fragment thereof are administered to the patient in dosages that range from one to five times per day or more. In other embodiments, the compositions comprising the syndecan-2 antibody or antigen-binding fragment thereof are administered to the patient in range of dosages that include, but are not limited to, once every day, every two, days, every three days to once a week, and once every two weeks. It is readily apparent to one skilled in the art that the frequency of administration of the various combination compositions comprising the syndecan-2 antibody or antigen-binding fragment thereof varies from individual to individual depending on many factors including, but not limited to, age, disease or disorder to be treated, gender, overall health, and other factors. Thus, the methods disclosed herein should not be construed to be limited to any particular dosage regime and the precise dosage and composition to be administered to any patient is determined by the attending physical taking all other factors about the patient into account.

A syndecan-2 antibody or antigen-binding fragment thereof may be administered to the subject in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a syndecan-2 antibody or antigen-binding fragment thereof is from about 1 mg and about 2,500 mg. In some embodiments, a dose of a syndecan-2 antibody or antigen-binding fragment thereof used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, also provided is a packaged pharmaceutical composition comprising a container holding a therapeutically effective amount of a syndecan-2 antibody or antigen-binding fragment thereof, including an anti-syndecan-2 antibody or a syndecan-2 disrupting peptide, alone or in combination with a second pharmaceutical agent; and instructions for using the compound to treat, prevent, or reduce one or more symptoms of a disease in a patient.

Formulations may be employed in admixtures with conventional excipients, i.e., pharmaceutically acceptable organic or inorganic carrier substances suitable for oral, parenteral, nasal, intravenous, subcutaneous, central, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the syndecan-2 antibody or antigen-binding fragment thereof, including the anti-syndecan-2 antibody or the syndecan-2 disrupting peptide, or compositions thereof include intravenous, intra-arterial, intra-muscular, intravitreal, subcutaneous, oral, nasal, rectal, intravaginal, parenteral, buccal, sublingual, intra-tumoral, or topical. The compositions comprising a syndecan-2 antibody or antigen-binding fragment thereof may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful herein are not limited to the particular formulations and compositions that are described herein.

### 6.8.1 Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated, or they may be coated by known techniques for elegance or to delay the release of the active ingredients. Formulations for oral use may also be presented as hard gelatin capsules wherein the active ingredient is mixed with an inert diluent.

Compositions comprising a syndecan-2 antibody or antigen-binding fragment thereof also includes a multi-layer tablet comprising a layer providing for the delayed release of one or more syndecan-2 antibody or antigen-binding fragment thereof, and a further layer providing for the immediate release of a medication for treatment of certain diseases or disorders. Using a wax/pH-sensitive polymer mix, a gastric insoluble composition may be obtained in which the active ingredient is entrapped, ensuring its delayed release.

### 6.8.2 Parenteral Administration

For parenteral administration, compositions comprising the syndecan-2 antibody or antigen-binding fragment thereof, including the anti-syndecan-2 antibody or the syndecan-2 disrupting peptide, may be formulated for injection or infusion, for example, intravenous, intravitreal, intra-arterial, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### 6.8.3 Additional Administration Forms

Additional dosage forms of compositions comprising the syndecan-2 antibody or antigen-binding fragment thereof, including the anti-syndecan-2 antibody or the syndecan-2 disrupting peptide, include dosage forms as described in U.S. Pat. Nos. 6,340,475; 6,488,962; 6,451,808; 5,972,389; 5,582,837; and 5,007,790. Additional dosage forms of compositions comprising the syndecan-2 antibody or antigen-binding fragment thereof also include dosage forms as described in U.S. Patents Applications Nos. 20030147952; 20030104062; 20030104053; 20030044466; 20030039688; and 20020051820. Additional dosage forms of compositions comprising the syndecan-2 antibody or antigen-binding fragment thereof also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of compositions comprising the syndecan-2 antibody or antigen-binding fragment thereof, including the anti-syndecan-2 antibody or the syndecan-2 disrupting peptide, may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the methods disclosed herein may be administered in the form of microparticles, for example, by injection or in the form of wafers or discs by implantation.

In one embodiment, the compounds are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### 6.8.4 Dosing

The therapeutically effective amount or dose of a composition comprising the syndecan-2 antibody or antigen-binding fragment thereof depends on the age, sex and weight of the patient, the current medical condition of the patient and the progression of a disease in the patient being treated. By way of a non-binding example, the dose of syndecan-2 antibody may be delivered as a single dose in response to an acute indication such as a stroke or myocardial infarction or the like. The skilled artisan is able to determine appropriate dosages depending on these and other factors.

A suitable dose of a syndecan-2 antibody or antigen-binding fragment thereof may be in the range of from about 0.01 mg to about 5,000 mg, such as from about 0.1 mg to about 1,000 mg, for example, from about 1 mg to about 500 mg, such as about 5 mg to about 250 mg. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the syndecan-2 antibody or antigen-binding fragment thereof is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the viral load, to a level at which the improved disease is retained. In certain embodiments, patients require intermittent treatment on a long-term basis upon any recurrence of symptoms and/or infection.

The composition comprising the syndecan-2 antibody or antigen-binding fragment thereof may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

### 6.9 Gene Therapy

In a specific embodiment, nucleic acids comprising sequences encoding Sdc2 antibodies provided herein, are administered to treat, prevent or ameliorate one or more symptoms associated with a disease or disorder provided herein by way of gene therapy. Gene therapy refers to therapy performed by the administration to a subject of an expressed or expressible nucleic acid. In this embodiment, the nucleic acids produce their encoded antibody that mediates a therapeutic or prophylactic effect.

Any of the methods for gene therapy available in the art can be used. Exemplary methods are described below and otherwise known in the art.

In one embodiment, a composition comprises nucleic acids encoding a Sdc2 antibody provided herein, said nucleic acids being part of an expression vector that expresses the antibody in a suitable host. In particular, such nucleic acids can have promoters, preferably heterologous promoters, operably linked to the antibody coding region, wherein the promoter can be inducible or constitutive, and, optionally, tissue-specific. In another particular embodiment, nucleic acid molecules are used in which the antibody coding sequences, and any other desired sequences are flanked by regions that promote homologous recombination at a desired site in the genome, thus providing for intrachromosomal expression of the antibody encoding nucleic acids.

Delivery of the nucleic acids into a subject may be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retroviral or other viral vectors, or by direct injection of naked DNA, or by use of microparticle bombardment (e.g., a gene gun), or coating with lipids (e.g., lipid nanoparticles (LNPs) or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor. Alternatively, the nucleic acid can be introduced intracellularly and incorporated within host cell DNA for expression, by homologous recombination.

In a specific embodiment, viral vectors that contain nucleic acid sequences encoding an Sdc2 antibody provided herein are used. For example, a retroviral vector can be used. These retroviral vectors can contain the components necessary for the correct packaging of the viral genome and integration into the host cell DNA. The nucleic acid sequences encoding the antibody to be used in gene therapy can be cloned into one or more vectors, which facilitates delivery of the nucleotide sequence into a subject.

Adenoviruses are other viral vectors that can be used in the gene therapy methods provided herein. Adenoviruses are especially attractive vehicles for delivering genes to respiratory epithelia. Other targets for adenovirus-based delivery systems are liver, the central nervous system, endothelial cells, and muscle. In other embodiments, an adeno-associated virus (AAV) is used for the gene therapy methods provided herein.

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject. In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell mediated gene transfer, spheroplast fusion, etc. Numerous techniques are known in the art for the introduction of foreign genes into cells and may be used in accordance with the methods provided herein, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny. The resulting recombinant cells can be delivered to a subject by various methods known in the art. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc. In certain embodiments, the cell used for gene therapy is autologous to the subject. In an embodiment in which recombinant cells are used in gene therapy, nucleic acid sequences encoding an antibody are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the gene therapy methods provided herein.

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

### 6.10 Methods of Detection and Kits

Anti-Sdc2 antibodies provided herein may also be used as agents to detect Sdc2-expressing cells. Thus, in another methods, provided is a method of detecting a cell expressing Sdc2, comprising contacting a cell with an anti-Sdc2 antibody provided herein. In certain embodiments, the detecting is by ELISA. In some embodiments, the detecting is by FACS analysis.

Also provided are kits comprising an anti-Sdc2 antibody provided herein, and instructions for use.

Exemplary binding agents that bind to Sdc2 are provided herein, for example in the Examples, as well as Tables 1, and 2-7.

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); and Antibody Engineering Vols 1 and 2 (Kontermann and Dübel eds., 2d ed. 2010).

Those skilled in the art will recognize or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

Particular embodiments of this invention are described herein. Upon reading the foregoing description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

As used herein, numerical values are often presented in a range format throughout this document. The use of a range format is merely for convenience and brevity and should not be construed as an inflexible limitation on the scope of the invention unless the context clearly indicates otherwise. Accordingly, the use of a range expressly includes all possible subranges, all individual numerical values within that range, and all numerical values or numerical ranges including integers within such ranges and fractions of the values or the integers within ranges unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. This applies regardless of the breadth of the range.

In addition, reference to a range of 1-3, 3-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-225, 225-250 includes 1, 2, 3, 4, 5, 6, 7, 8, 9, 10, 11, 12, 13, 14, 15, 16, 17, 18, 19, 20, etc. In a further example, reference to a range of 25-250, 250-500, 500-1,000, 1,000-2,500, 2,500-5,000, 5,000-25,000, 25,000-50,000 includes any numerical value or range within or encompassing such values, e.g., 25, 26, 27, 28, 29 . . . 250, 251, 252, 253, 254 . . . 500, 501, 502, 503, 504 . . . , etc.

As also used herein a series of ranges are disclosed throughout this document. The use of a series of ranges includes combinations of the upper and lower ranges to provide another range. This construction applies regardless of the breadth of the range and in all contexts throughout this patent document. Thus, for example, reference to a series of ranges such as 5-10, 10-20, 20-30, 30-40, 40-50, 50-75, 75-100, 100-150, includes ranges such as 5-20, 5-30, 5-40, 5-50, 5-75, 5-100, 5-150, and 10-30, 10-40, 10-50, 10-75, 10-100, 10-150, and 20-40, 20-50, 20-75, 20-100, 20-150, and so forth.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

A number of embodiments of the invention have been described. Nevertheless, it will be understood that various modifications may be made without departing from the spirit and scope of the invention. Accordingly, the descriptions in the Examples section are intended to illustrate but not limit the scope of invention described in the claims.

### 7. EXAMPLES

Certain aspects and embodiments provided herein are now described with reference to the following Examples. These Examples are provided for the purpose of illustration only, and the invention is not limited to these Examples, but rather encompasses all variations that are evident as a result of the teachings provided herein.

The materials and methods employed in the experimental examples disclosed herein are now provided.

PentaMice hybridoma generation. Immunization of mice was performed by HT-Hock™ immunization of 2 cohorts of PentaMice™ (5 strains, 10 mice, 2 mice per strain). The first cohort were mice immunized with huSdc2 (extracellular domain, (ECD)). An optional final boost with human and pigAG3-KLH (peptide AG3 conjugated with KLH) was performed depending on measured titers. The second cohort were mice immunized with KLH-conjugated AG3 (huAG3 and pAG3). An optional final boost with either KLH-conjugated AG3 mixture or huSdc2 (ECD) was performed based on the observed titer. Plasma titers were tested by ELISA against huSdc2(ECD)-His, irrelevant control protein-His (to evaluate anti-His response), and BSA-conjugated AG3 peptides. The composition of the final boost (3 days before harvest and fusion) will be dependent on titers. Lymph nodes and spleens will be collected based on titer. For hybridoma generation, lymphocytes and/or splenocytes were pooled (as appropriate based on titer) and subjected to 1-2 electro fusions. The resulting hybridomas were utilized as described below in “Antibody Recovery”.

Antibody Recovery. Fused hybridomas were plated in 10×384-well plates at a target density of 1 to 3 hybridomas per well. Unfused immunized material, as well as unplated fusion material, were cryopreserved. The hybridomas were first subjected to a primary screen consisting of enzyme-linked immunoassay (ELISA) against huSdc2-ECD-His followed by selection and expansion of ELISA-positive hybridomas. A secondary screen was then performed using ELISA against huSdc2-ECD-His followed by another round of expansion and cryopreservation of ELISA-positive hybridomas. Cultures were then used to generate and harvest non-clonal saturated supernatants (2 mL). Lastly a tertiary screen was performed with an ELISA against huSdc2-ECD, an ELISA against huSdc2-Fc, an ELISA against BSA-conjugated huAG3 peptide, an ELISA against BSA-conjugated pAG3 peptide, an ELISA against other BSA-conjugated Sdc2 peptides, and an ELISA against a counter-screen antigen (Sdc4), and an ELISA against a counter-screen irrelevant protein-His.

Humanization of mAbs (monoclonal antibodies) from PentaMice. Humanization of mouse antibody sequences was performed by complementarity-determining regions (CDR) grafting, in which CDRs were transferred into human antibody framework regions while retaining original affinity and specificity. The LakePharma T20 Score Analyzer tool was then used to calculate a monoclonal antibody humanness score to distinguish between human and non-human antibodies (Gao, et al. (2013) BMC Biotechnology, 13:55).

Functional validation of mAbs. The evaluation of mAbs biological activity was assessed by an in vitro permeability assay. The assay was performed with the xCELLigence RTCA System (Agilent) which allows measurement of endothelial layer integrity (expressed as electrical impedance) in response to VEGFA. Briefly, 20000 Human Umbilical Vein Endothelial Cells (HUVEC) cells/well were seeded onto E-Plate 16 (Agilent #5469830001) in 200 μl of EGM2-MV medium for 48 hrs. Cells were then starved 8 hrs. in 5% FBS growth factor-free medium (EBM2) and then stimulated with VEGFA165 (100 ng/ml) for up to 72 hrs. Each tested mAb was added 1 hr. before (prevention) or 3 hrs. after (therapy) VEGFA stimulation to evaluate inhibition of VEGFA-induced drop in electrical impedance (Delta cell index), which is an indicator of ‘vascular leakage’ and/or ‘increase in endothelial permeability’).

### 7.1 Example 1: Development of Humanized Anti-Sdc2 Antibodies

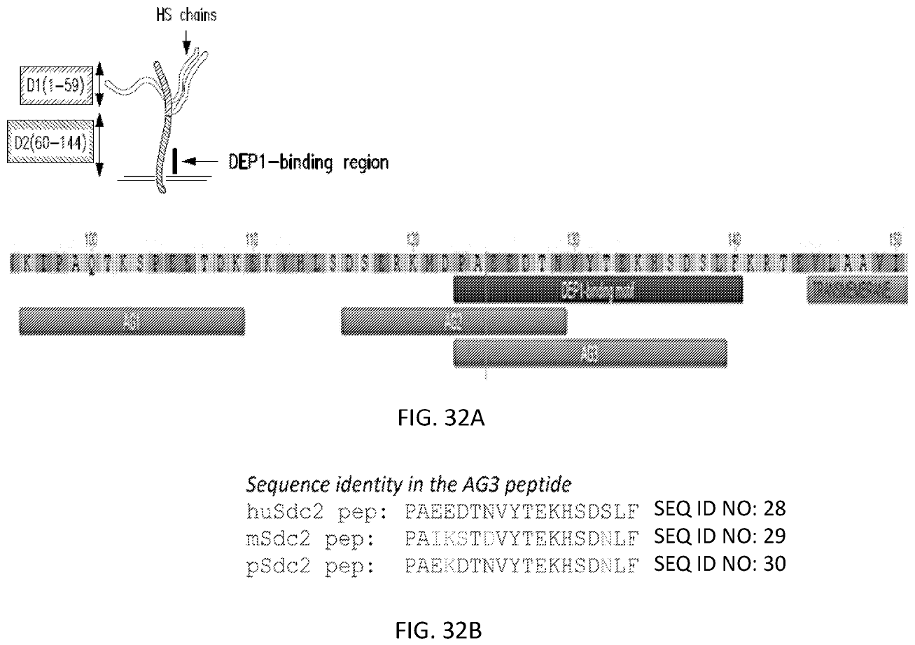

The studies presented in the current disclosure were undertaken in order to generated fully human antibodies specific for human Sdc2 (Syndecan-2, CD362, NCBI accession AAH49836). Sdc2 protein is a transmembrane (type I) heparan sulfate proteoglycan and is a member of the Syndecan proteoglycan family1. Human Sdc2 is expressed as a 201 amino acid (aa) core protein with an 18 aa signal sequence, a 126 aa extracellular domain, a 25 aa transmembrane region and a 32 aa cytoplasmic tail. Sdc2 carries three long negatively charged heparan sulfate chains. Within the ECD is the DEP1-binding domain, which can be represented by “AG3 peptide” (PAEEDTNVYTEKHSDSLF) (SEQ ID NO: 28). Human (huSdc2) and murine (mSdc2) Sdc2 share 77.6% sequence identity between their extracellular domains and 72.2% between their AG3 peptide sequences (FIG. 32). Human and porcine (pSdc2) Sdc2 share 92.86% sequence identity between their extracellular domains and 88.9% identity between their AG3 peptide sequences. The syndecan-2 protein functions as an integral membrane protein and participates in cell proliferation, cell migration and cell-matrix interactions. Previous studies have demonstrated that its expression is significantly upregulated in several cancer cell lines and in some human cancers.

Studies of the present disclosure were undertaken in order to develop humanized antibodies with strong functional binding to hSdc2 and cross-reactive binding to pSdc2 and mSdc2. Candidate clones were also selected to have no cross-reactivity to Sdc4. The PentaMice™ platform from LakePharma (now Curia) was utilized for these studies. This platform comprises a set of mice which includes five wildtype mouse strains that cover nine distinct MHC haplotypes. The combination of several genetically wildtype mice enables the generation of a wider repertoire of possible antibodies following immunization with an antigen of interest (e.g., Sdc2), as opposed to traditional murine systems that use a single inbred strain.

One cohort of PentaMice™ (10) each were immunized with huSdc2(ECD)-His (Cohort-1; M1-10) and KLH-conjugated AG3 peptide mix of huAG3 and pAG3 (Cohort-2; M11-20) via HT-Hock method. Immunizations were continued for both the Cohorts for one more week to improve the Day 24 titers. No final boost was performed for mice M5 and 6 as the titers were low and they were euthanized. Mice M1, 2, 3, 4, 7, 8, 9, and 10 were boosted with a mixture of huAG3 and pAG3, and mice M11, 13, 14, 15, 16, 17, 19, and 20 were boosted with hSdc2(ECD)-His, 3 days prior to harvesting splenocytes and lymphocytes (LN) for fusion. Mice M12 and M18 died before boosting, probably due to cytokine storm (very high titers).

Two separate electrofusions were performed from the mice showing the best titers. Up to 35M Lymphocytes pooled from Mice M4 and M9 (Cohort-1) and from Mice M13, 14, 15, 19, and 20 (Cohort-2) were used for fusion-1 and fusion-2, respectively, and frozen before thawing and plating in 10×384 well plates (1-5: Cohort-1; and 6-10: Cohort-2). The fusion efficiency was less than expected, and the plates were screened against hSdc2(ECD)-His. From ELISA primary screen, 142 hits were picked (>20,000 RLU; 117 from Cohort-1 and 25 from Cohort-2). 39 out of these 142 hits were confirmed (>50,000 RLU; 29 from Cohort 1 and 10 from Cohort 2).

Another round of fusions (with splenocytes) was performed and plated in 10×384 well plates (Mouse 11-15: Cohort-1 and Mouse 16-20: Cohort-2). Fusion efficiency was good and within our expected range, with observed efficiency of 3.5 and 2.7, for Cohort-1 and Cohort-2, respectively. The plates were screened against hSdc2(ECD)-His as well as huAG3-BSA and huSdc2(ECD)-hFc according to the following workflow.

### Primary Screen:

ELISA against huSdc2-ECD-His (*included ELISA against huAG3 peptide as well as huSdc2(ECD)-hFc)
Select and expand ELISA-positive hybridomas

### Secondary Screen:

ELISA against huSdc2-ECD-His (*include ELISA against huAG3 peptide as well as huSdc2(ECD)-hFc).
Expand and cryopreserve ELISA-positive hybridomas
Generate and harvest non-clonal saturated supernatants

### Tertiary Screen:

ELISA against huSdc2-ECD
ELISA against huSdc2-Fc.
ELISA against BSA-conjugated huAG3 peptide
ELISA against BSA-conjugated pAG3 peptide
ELISA against other BSA-conjugated Sdc2 peptides. * Only huAG3 and pAG3 peptides would be tested.
ELISA against counter-screen antigen (Sdc4).
ELISA against counter-screen irrelevant protein-His

From the ELISA primary screen, 82 hits were picked (>20,000 RLU on huSdc2(ECD)-His, huAG3-BSA and huc2-Fc). 19 out of these 82 hits were confirmed (>50,000 RLU).

All the 59 confirmed clones from both the fusions were cryopreserved as well as expanded to collect 2 ml of non-clonal saturated supernatant for Tertiary screening. Tertiary screen was performed for 55/58 Hybridomas by ELISA against huSdc2-ECD, huSdc2-Fc, huAG3-BSA, pAG3-BSA, Sdc4 counter and irrelevant His counter. huSdc2-ECD, huSdc2-Fc, Sdc4 counter and irrelevant His counter were coated at 1 ug/ml; huAG3-BSA and pAG3-BSA were coated at 5 ug/ml. 19838-8-K02, 19838-18-F21 and 19838-18-G02 failed to expand. 44/55 hybridomas showed binding against huSdc2-ECD, huSdc2-Fc, huAG3-BSA or pAG3-BSA with different profiles (cutoff: ≥50,000RLU)·
10/55 are non-specific binders or negative hybridomas.
12/44 bind to huSdc2-ECD, huSdc2-Fc and huAG3-BSA, and cross reactive pAG3-BSA
1/44 bind to huSdc2-ECD, huSdc2-Fc and huAG3-BSA with no cross reactivity to pAG3-BSA
24/44 only bind to huSdc2-ECD and huSdc2-Fc proteins
6/44 bind to both huAG3-BSA and pAG3-BSA peptides with no binding to huSdc2 proteins
1/44 only bind to huAG3-BSA

Each clone of these 55 non-clonal saturated supernatants were further tested using functional assays, and based on these results, only 5 out 55 of the non-clonal hybridomas (clones id #20-H19-A, 6-N03-A, 8-G17-A, 6-E17-A and 10-I12-A) were chosen for sub-cloning, variable region sequencing and recombinant production.

Selected hybridomas were then subjected to variable region sequencing. Antibody sequence analysis identified single heavy and light chains for four of the five submitted sequencing samples, and one hybridoma has a single heavy chain and two light chains. A diverse set of mAbs was discovered comprising one unique antibody in two sibling groups. In terms of overall diversity, three types of anti-Sdc2-binding antibodies were identified based on sequence analysis. For 20-H19-A, a single heavy chain and 2 light chains were obtained (recombinant production of each clone (20-H19-AA and 20-H19-AB) is made to evaluate functional activity). Sib1 group comprises of two members (6-E17-A and 10-I12-A) with identical V and J usage for both heavy and light chains and with a single amino acid difference in the CDR3 region of both heavy and light chains. Sib2-Id1 group comprises of two members (6-N03-A and 8G17-A) with identical V, J and CDR3 domains for both heavy and light chains. Only one amino acid difference in the CDR1 domain of light chain was observed.

Selected mAbs were then purified and characterized. The selected clones (20-H19-AA and 20-H1-AB, 6-N03-A, 8-G17-A, 6-E17-A and 10-I12-A) were recombinantly produced in CHO cells. Purified monoclonal antibodies (mAbs) were then evaluated with epitope binning analysis alongside with other candidates from XOMA library panning (see Example 3). Following in-house functional analysis, significant biological activity was confirmed for the following clones: 20-H1-AB, 8-G17-A, 6-N03-A.

Hybridomas expressing antibody clones which passed screening were then humanized by complementarity-determining regions (CDR) grafting, in which CDRs were transferred into human antibody framework regions while retaining original affinity and specificity. The LakePharma T20 Score Analyzer tool was then used to calculate a monoclonal antibody humanness score to distinguish between human and non-human antibodies (Gao, et al. (2013) BMC Biotechnology, 13:55).

The VH and VL sequences of certain Sdc2 antibodies are provided in Table 1 and Table 3. The CDRs sequences of certain Sdc2 antibodies are provided in Table 4 (Kabat), Table 5 (Chothia), Table 6 (AbM), Table 7 (Contact), and Table 8 (IMGT). Respective SEQ ID NOs are provided below each sequence.

TABLE 1
Amino Acid Sequences of Sdc2 Antibody Heavy Chain and
Light Chain Variable Regions

| | SEQ | | |
|---|---|---|---|
| | ID | | |
| # | NO: | Name: | Sequence: |
| 1 | 1 | 20-H19-AB | EVQLVESGGGLVQPKGSLKLSCAASGFTFNIYAMHWVRQAPGKGLEWIARIGSKS |
| | | Heavy Chain | SYYATYYADSVKDRFTISRDDSQSMLYLQTNNLKTEDTAMYYCVSGKGDWGQGTT |
| | | | LTVSS |
| | 2 | 20-H19-AB | DVVMTQTPLTLSVIIGQPASISCKSSQSLLAGDGKTYLNWLLQRPGQSPKRLIYL |
| | | Light Chain | VSKLDSGVPDRFTGSGSGTDFTLKISRVEAEDLGVYYCWQGTHFPWTFGAGTRLE |
| | | | IK |
| 2 | 3 | TP-43327F- | QVQLVESGGGVVQPGGSLRLSCAASGFTFNIYAMHWVRQAPGKGLEWVARIGSKS |
| | | Heavy Chain | SYYATYYADSVKGRFTISRDNSKNTLYLQMNSLRTEDTAVYYCVSGKGDWGQGTL |
| | | | VTVSS |
| | 4 | TP-43327F- | DVVMTQSPLSLPVTLGQPASISCRSSQSLLAGDGKTYLNWFLQRPGQSPRRLIYL |
| | | Light Chain | VSKLDSGVPDRFSGSGSGTDFTLKISRVEAEDVGVYYCWQGTHFPWTFGAGTRLE |
| | | | IK |
| 3 | 5 | TP-43329F- | QVQLVESGGGVVQPGGSLRLSCAASGFTFNIYAMHWVRQAPGKGLEWVARIGSKS |
| | | Heavy Chain | SYYATYYADSVKGRFTISRDNSKNTLYLQMNSLRTEDTAVYYCVSGKGDWGQGTL |
| | | | VTVSS |
| | 6 | TP-43329F- | DVVMTQSPLSLPVTLGQPASISCRSSQSLLAGDGKTYLNWFQQRPGQSPRRLIYL |
| | | Light Chain | VSKLDSGVPDRFSGSGSGTDFTLKISRVEAEDVGVYYCWQGTHFPWTFGAGTRLE |
| | | | IK |
| 4 | 7 | 8-G17-A- | EVQLVESGGGLVKPGGSLKLSCEASGITFSNYVMSWVRQTPEKRLEWVATISDGG |
| | | Heavy Chain | SYTYYPDNLKGRFTISRDNAKNNLYLQMSHLKSEDTAMYYCVRDRYDYDGRVYAM |
| | | | DYWGQGTSVTVSS |
| | 8 | 8-G17-A | DVVMTQTPLSLPVSLGDQASISCRSSQSLVHSNGNTYLHWYLQKPGQSPKLLIYK |
| | | Light Chain | VSNRFSGVPDRFSGSGSGTDFTLKITRVEAEDLGVYFCSQSTHVPWTFGGGTKLE |
| | | | IK |
| 5 | 9 | 6-N03-A- | EVQLVESGGGLVKPGGSLKLSCEASGITFSNYVMSWVRQTPEKRLEWVATISDGG |
| | | Heavy Chain | SYTYYPDNVKGRFTISRDNAKNNLYLQMSHLKSEDTAMYYCVRDRYDYDGRVYAM |
| | | | DYWGQGTSVTVSS |
| | 10 | 6-N03-A- | DVVMTQTPLSLPVSLGDQASISCRSSQSLVHSNGDTYLHWYLQKPGQSPKLLIYK |
| | | Light Chain | VSNRFSGVPDRFSGSGSGTDFTLKITRVEAEDLGVYFCSQSTHVPWTFGGGTKLE |
| | | | IK |
| 6 | 11 | R3-P3-C11- | EVQLVESGGGLVQPGRSLRLACEASGFTFNNYAVHWVRQAPGKGLEWVAVISYDG |
| | | Heavy Chain | TNKYYSDSMKGRFTISRDNSKNTLYLQINSLRVEDTAVYFCARGNYVRRDSFDIW |
| | | | GQGTMVTVSS |
| | 12 | R3-P3-C11- | DIQMTQSPPSLSASVGDRVTITCRASQSISSYLNWYQQKPGKAPKLLIYGASSLQ |
| | | Light Chain | SGVPLRFSGSGSGADFTLTISSLQPEDFATYYCQQSYISPITFGQGTRLEIK |
| 7 | 13 | R4M-P3-E06- | EVQLVESGGGVVQPGGSLRLSCAASGFTFSNYGMHWVRQAPGKGLEWVATISYDG |
| | | Heavy Chain | TKKYYPDSVRGRFTISRDNSENMLYLQMDSLRLEDTAVIYCAKDLVSGSRYGFAF DSWGPGTLVTVSS |
| | 14 | R4M-P3-E06- | DIVMTQSPLSLPVTLGQPASISCRSSQSLVHSDGNTYLNWFQQRPGQSPRRLIYK |
| | | Light Chain | VSNRDSGVPDRFSGTGSGTDFTLKISRVEADDVGVYYCMQGTHWPPIFGQGTRLE IK |
| 8 | 15 | R3-P3-E09 | EVQLVETGGGVVQPGRSLTLSCGASEFTFSNYAMHWVRQAPGKGLEWVAVISYDG |
| | | Heavy Chain | TKKYYGDSVKGRFTISRENIKNTLYLQMNSLRSEDTAVYYCARDVGKGVTATGTF |
| | | | DIWGQGTMVTVSS |
| | 16 | R3-P3-E09- | AIRMTQSPSSLSASVGDRVTITCRASQGIGSYLAWFQQKPGEAPKSLIIDATRLK |
| | | Light Chain | SGVPSRFSGSGSGTEFALTISSLRPEDFATYYCQQYNVSPLTFGGGTKVEIK |
| 9 | 17 | R3-P1-C02- | EVQLVESGGGVVQPGGSLRISCVASEFPFSDFGMHWVRQAPGKGLEWVAFVRSHG |
| | | Heavy Chain | SGKYYADSVKGRFTISGDKSKSTLYLQMESLRPDDAAVYYCTTLADVWGQGTTVT |
| | | | VSS |
| | 18 | R3-P1-C02 | DVVMTQSPLSLPVTLGQPASISCRSSQSLVYSDGNTYLNWFQQRPGQSPRRLIYK |
| | | Light Chain | VSNRDSGVPDRFSGSGSGTDFTLKISRVEAEDVGVYYCMQGTHWPWTFGQGTKVE |
| | | | IK |
| 10 | 19 | R3-P3-A12- | EVQLVESGGGLVQPGRSLRLSCGVSGFTFSSFGMHWVRQAPGKGLEWVAVISNDA |
| | | Heavy Chain | LHKNYGDSVKGRFTVTRDNSRNTLFLQMTSLRPEDTAVYYCAKDVVRGHTSGSLG |
| | | | SWGQGTLVTVSS |
| | 20 | R3-P3-A12- | DVVMTQSPLSLPVTPGEPASISCRSSQSLLYSNGYNYLDWYLQKPGQSPQLLIYL |
| | | Light Chain | GSNRASGVPDRFSGSGSGTDFTLKISRVEAEDVGVYYCMQALQTPLTFGGGTKLE |
| | | | IK |
| 11 | 21 | R4M-P3-A12 | EVQLVESGGGVVQPGGSLRLSCAASGFTFSDHYMSWIRQAPGKGLEWVSYLSGSG |
| | | Heavy Chain | TSIYYADSVKGRFTISRDNAKKSLYLQMNSLRAEDTAVYYCARVGPAAGNAFDIW |
| | | | GQGTMVTVSS |
| | 22 | R4M-P3-A12- | QSVLTQPPSASGTPGQRVTISCSGSSSNIGSNYVYWYQQLPGTAPKLLIYRNNQR |
| | | Light Chain | PSGVPDRFSGSKSGTSASLAITGLQAEDEADYYCQSYDSSLSGVVFGGGTKLTVL |
| 12 | 23 | R4M-P1-A10- | EVQLVETGGGVVQPGRSLRLSCAASGFTFSSYAMHWVRQAPGKGLEWVAVISYDG |
| | | Heavy Chain | SNKYYADSVKGRFTISRDNSKNTLYLQMNSLRAEDTAVYYCARDRGWADAFDIWG QGTMVTVSS |
| | 24 | R4M-P1-A10- | QAGLTQPPSVSKGLRQTATLTCTGNSNNVGYQGAVWLLQYQGHPPKVLSNRNNDR |
| | | Light Chain | PSGISERLSASRSGNTASLTITGLQPEDEGDYFCAAWDGSLRGWVFGGGTKLTVL |

### 7.2 Example 2: Development of Fully Human Sdc2 Antibodies

In addition to humanized mouse antibodies, studies disclosed herein were also undertaken to generate and identify fully human anti-Sdc2 antibodies using an scFv human naïve XOMA phage display library. High affinity scFv antibodies that bind huSdc2 and recognize the DEP1-binding domain were generated.

Candidate clones for further development displayed cross-reactivity to mouse Sdc2 (Uniprot P43407) and pig Sdc2 (NP_001302683.1). A panel of Sdc2 extracellular binding domain (ECD) binders were generated and subjected to further characterization of binding affinity and functional ability (e.g., blocking Sdc2 function). Of particular interest were those clones able to bind AG3 peptide (FIG. 32B).

The panning strategy (Table 2) focused on identifying huSdc2 ECD binders using the human naïve scFv XOMA library that are also able to recognize the AG3 peptide (Arm A) and cross-reactive to mSdc2 (Arm B). Briefly, the XOMA naïve scFv library was allowed to bind to huSdc2 coated on Maxisorp ELISA wells. This selection step was allowed to proceed for 1 h at RT. Phage-bound to huSdc2 was then washed in PBST, eluted and used for the 2nd round of panning also performed with huSdc2. A 3rd round of panning was then performed using the output phage from the 2nd Rd vs huSdc2. To allow the discovery of hSdc2-specific clones that are also able to recognize the AG3 peptide, under Arm A, a 4th round of panning was performed using BSA-AG3 immobilized on a Maxisorp plate, while deselecting against potential non-specific binders using BSA alone. To also allow for the discovery of mSdc2 cross-reactive binders, under Arm B, a 4th round of panning was performed using mSdc2 immobilized on a Maxisorp plate.

TABLE 2
XOMA Panning Strategy

| Panning | | | | |
|---|---|---|---|---|
| Rd | Deselection | Arm A | Arm B | Library |
| 1 | N/A | huSdc2 | huSdc2 | XOMA scFv |
| 2 | N/A | huSdc2 | huSdc2 | R1 output |
| 3 | N/A | huSdc2 | huSdc2 | R2 output |
| 4 | BSA | BSA-hAG3 | mSdc2 | R3 output |
| | | peptide | | |
| Goal | | Selection of | Selection of | |
| | | hAG3 specific | hu/m Sdc2 | |
| | | binders | binders (pSdc2 | |
| | | | cross-reactivity | |
| | | | will be | |
| | | | assessed in | |
| | | | tertiary screens | |

Pools of phage were then screened by ELISA against huSdc2 and possibly BSA-AG3 to assess enrichment between successive rounds of panning. Panned outputs from library and round showing are moved forward for screening.

After 4 rounds of panning, phage pool ELISA results showed good enrichment for human and mouse Sdc2 in rounds 3 and 4. No enrichment for human Ag3 peptide was observed. HuAg3 peptide panning was revisited and huAg3 peptide was introduced sooner for one additional round of panning. The round 2 output was used as input to pan against huAg3. Output titer was low and phage pool ELISA results did not show enrichment. 1×96 well scouting plate of PPE was screened from R4M against huSdc2, muSdc2 and huAg3 peptides, which showed twelve clones to have good binding (>3× over background) and were thus submitted for sequencing. Overall, six sequence unique binders were identified with one clone, E08, appearing to bind the huAg3 peptide.

For screening and sequencing studies that followed panning, 8×96-well plates of output clones were setup as master plates. All ELISA screens utilized Maxisorp plates, and bacterial periplasmic extracts (PPEs) or phage of output clones were utilized for screening. First, an ELISA primary screen comprising eight plates was conducted against hSdc2. This was followed by a series of secondary screens in which the top hSdc2-positive hits from both Arms A and B of panning were screened by ELISA against BSA-hAG3, mSdc2 and BSA alone (counter-screen). All mSdc2 and BSA-hAG3 cross-reactive clones were then re-arrayed and subjected to a tertiary round of ELISA screens. Up to 4×96 positive clones were sequenced and analyzed to identify sequence-unique clones. Sequence liability analysis was performed on the top hits which comprised identification of amino acid motifs that are sensitive to post-translational modifications (e.g., deamination, glycosylation, free cysteines). As a result, up to 2×96 target-binding, sequence unique clones were re-arrayed. New PPEs were generated and assayed as follows: ELISA against pSdc2, ELISA against BSA-mAG3, and kinetic analysis by arrayed SPR using Carterra LSA.

In total, 8×96-well plates of PPEs (752 individual clones) were screened against HuSdc2, MoSdc2, huAg3, and BSA. Thirty-six unique clones were identified after sequence analysis of which eleven clones were moSdc2 cross reactive. The 36 unique clones were re-arrayed and additional PPE were generated for tertiary screens and kinetic analysis. PPE of these 36 unique clones were also selected for further functional assays. Counter-screening of unique clones was completed against all antigens: HuSdc2, MoSdc2, huAg3, moAg3, PigSdc2, HuSdc2-Fc. HuSdc2-Fc was added to the counter-screen to verify clone binding to two antigen formats. Of these 31 or 36 clones were reconfirmed to bind HuSdc2-His by ELISA, and 17 of 36 clones were found to bind both HuSdc2 antigen formats. Of these 17 clones, all bound pigSdc2 and 10 of 17 bound moSdc2.

Ten lead candidates from the phage campaign were reformatted as fully human antibodies (hIgG1) and recombinantly expressed using the 0.01 L TunaCHO 14-day process and purified using Protein A resin affinity purification. Purified monoclonal antibodies (mAbs) were then evaluated with epitope binning analysis alongside with other mouse hybridoma candidates from Example 1 (see Example 3).

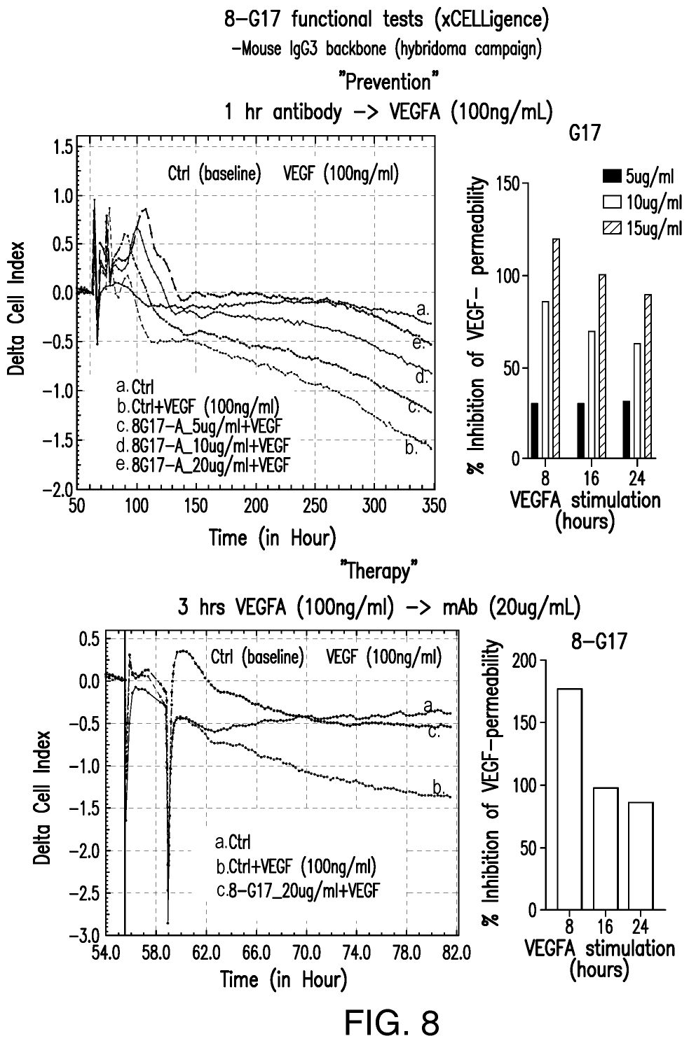

Following functional analysis, significant biological activity was confirmed for the following clones: R3-P3-C11, R4M-P3-E06, R3-P3-E09, R3-P1-C02, R3-P3-A12, R4M-P3-A12, R4M-P1-A10 (see FIGS. 8-26 for details about binding affinity and functional activity

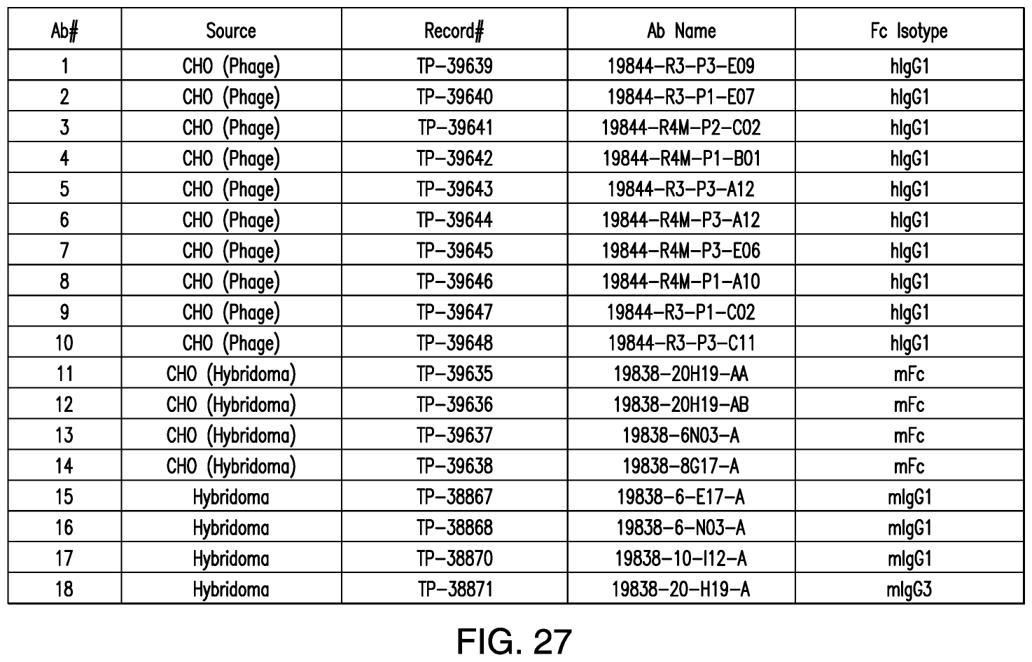

The antibodies created and selected for further development and their source (mouse hybridoma or phage library) are listed in FIG. 27 and the sequences of their respective heavy and light chains are disclosed in Table 1. The VH and VL sequences of certain Sdc2 antibodies are provided Table 3. The CDRs sequences of certain Sdc2 antibodies are provided in Table 4 (Kabat), Table 5 (Chothia), Table 6 (AbM), Table 7 (Contact), and Table 8 (IMGT). Respective SEQ ID NOs are provided below each sequence.

TABLE 3
Anti-Sdc2 Antibody VH and VL Amino Acid Sequences

| | | HC | LC | | | | |
|---|---|---|---|---|---|---|---|
| | Protein | Iso- | Iso- | VH AA | VL AA | | Light Chain AA |
| # | Name | type | type | sequence | sequence | Heavy Chain AA sequence | sequence |
| 1 | 20-H19- | IgG1 | Kappa | EVQLVESGGG | DVVMTQTPL | MDPKGSLSWRILLFLSLAFELSYGEVQLVESGG | METDTLLLWVLLLWVP |
| | AB | | | LVQPKGSLKL | TLSVIIGQP | GLVQPKGSLKLSCAASGFTFNIYAMHWVRQAPG | GSTGDVVMTQTPLTLS |
| | | | | SCAASGFTFN | ASISCKSSQ | KGLEWIARIGSKSSYYATYYADSVKDRFTISRD | VIIGQPASISCKSSQS |
| | | | | IYAMHWVRQA | SLLAGDGKT | DSQSMLYLQTNNLKTEDTAMYYCVSGKGDWGQG | LLAGDGKTYLNWLLQR |
| | | | | PGKGLEWIAR | YLNWLLQRP | TTLTVSSAKTTPPSVYPLAPGSAAQTNSMVTLG | PGQSPKRLIYLVSKLD |
| | | | | IGSKSSYYAT | GQSPKRLIY | CLVKGYFPEPVTVTWNSGSLSSGVHTFPAVLQS | SGVPDRFTGSGSGTDF |
| | | | | YYADSVKDRF | LVSKLDSGV | DLYTLSSSVTVPSSTWPSETVTCNVAHPASSTK | TLKISRVEAEDLGVYY |
| | | | | TISRDDSQSM | PDRFTGSGS | VDKKIVPRDCGCKPCICTVPEVSSVFIFPPKPK | CWQGTHFPWTFGAGTR |
| | | | | LYLQTNNLKT | GTDFTLKIS | DVLTITLTPKVTCVVVDISKDDPEVQFSWFVDD | LEIKRADAAPTVSIFP |
| | | | | EDTAMYYCVS | RVEAEDLGV | VEVHTAQTQPREEQFNSTFRSVSELPIMHQDWL | PSSEQLTSGGASVVCF |
| | | | | GKGDWGQGTT | YYCWQGTHF | NGKEFKCRVNSAAFPAPIEKTISKTKGRPKAPQ | LNNFYPKDINVKWKID |
| | | | | LTVSS | PWTFGAGTR | VYTIPPPKEQMAKDKVSLTCMITDFFPEDITVE | GSERQNGVLNSWTDQD |
| | | | | | LEIK | WQWNGQPAENYKNTQPIMDTDGSYFVYSKLNVQ | SKDSTYSMSSTLTLTK |
| | | | | | | KSNWEAGNTFTCSVLHEGLHNHHTEKSLSHSPG | DEYERHNSYTCEATHK |
| | | | | | | | TSTSPIVKSFNRNEC |
| | (SEQ ID | | | 61 | 62 | 63 | 64 |
| | NOs:) | | | | | | |
| 2 | TP- | IgG1 | Kappa | QVQLVESGGG | DVVMTQSPL | MDPKGSLSWRILLFLSLAFELSYGQVQLVESGG | METDTLLLWVLLLWVP |
| | 43327F | | | VVQPGGSLRL | SLPVTLGQP | GVVQPGGSLRLSCAASGFTFNIYAMHWVRQAPG | GSTGDVVMTQSPLSLP |
| | | | | SCAASGFTFN | ASISCRSSQ | KGLEWVARIGSKSSYYATYYADSVKGRFTISRD | VTLGQPASISCRSSQS |
| | | | | IYAMHWVRQA | SLLAGDGKT | NSKNTLYLQMNSLRTEDTAVYYCVSGKGDWGQG | LLAGDGKTYLNWFLQR |
| | | | | PGKGLEWVAR | YLNWFLQRP | TLVTVSSASTKGPSVFPLAPSSKSTSGGTAALG | PGQSPRRLIYLVSKLD |
| | | | | IGSKSSYYAT | GQSPRRLIY | CLVKDYFPEPVTVSWNSGALTSGVHTFPAVLQS | SGVPDRFSGSGSGTDF |
| | | | | YYADSVKGRF | LVSKLDSGV | SGLYSLSSVVTVPSSSLGTQTYICNVNHKPSNT | TLKISRVEAEDVGVYY |
| | | | | TISRDNSKNT | PDRFSGSGS | KVDKKVEPKSCDKTHTCPPCPAPELLGGPSVFL | CWQGTHFPWTFGAGTR |
| | | | | LYLQMNSLRT | GTDFTLKIS | FPPKPKDTLMISRTPEVTCVVVDVSHEDPEVKF | LEIKRTVAAPSVFIFP |
| | | | | EDTAVYYCVS | RVEAEDVGV | NWYVDGVEVHNAKTKPREEQYNSTYRVVSVLTV | PSDEQLKSGTASVVCL |
| | | | | GKGDWGQGTL | YYCWQGTHF | LHQDWLNGKEYKCKVSNKALPAPIEKTISKAKG | LNNFYPREAKVQWKVD |
| | | | | VTVSS | PWTFGAGTR | QPREPQVYTLPPSREEMTKNQVSLTCLVKGFYP | NALQSGNSQESVTEQD |
| | | | | | LEIK | SDIAVEWESNGQPENNYKTTPPVLDSDGSFFLY | SKDSTYSLSSTLTLSK |
| | | | | | | SKLTVDKSRWQQGNVFSCSVMHEALHNHYTQKS | ADYEKHKVYACEVTHQ |
| | | | | | | LSLSPG | GLSSPVTKSFNRGEC |
| | (SEQ ID | | | 95 | 96 | 97 | 98 |
| | NOs:) | | | | | | |
| 3 | TP- | IgG1 | Kappa | QVQLVESGGG | DVVMTQSPL | MDPKGSLSWRILLFLSLAFELSYGQVQLVESGG | METDTLLLWVLLLWVP |
| | 43329F | | | VVQPGGSLRL | SLPVTLGQP | GVVQPGGSLRLSCAASGFTFNIYAMHWVRQAPG | GSTGDVVMTQSPLSLP |
| | | | | SCAASGFTFN | ASISCRSSQ | KGLEWVARIGSKSSYYATYYADSVKGRFTISRD | VTLGQPASISCRSSQS |
| | | | | IYAMHWVRQA | SLLAGDGKT | NSKNTLYLQMNSLRTEDTAVYYCVSGKGDWGQG | LLAGDGKTYLNWFQQR |
| | | | | PGKGLEWVAR | YLNWFQQRP | TLVTVSSASTKGPSVFPLAPSSKSTSGGTAALG | PGQSPRRLIYLVSKLD |
| | | | | IGSKSSYYAT | GQSPRRLIY | CLVKDYFPEPVTVSWNSGALTSGVHTFPAVLQS | SGVPDRFSGSGSGTDF |
| | | | | YYADSVKGRF | LVSKLDSGV | SGLYSLSSVVTVPSSSLGTQTYICNVNHKPSNT | TLKISRVEAEDVGVYY |
| | | | | TISRDNSKNT | PDRFSGSGS | KVDKKVEPKSCDKTHTCPPCPAPELLGGPSVFL | CWQGTHFPWTFGAGTR |
| | | | | LYLQMNSLRT | GTDFTLKIS | FPPKPKDTLMISRTPEVTCVVVDVSHEDPEVKF | LEIKRTVAAPSVFIFP |
| | | | | EDTAVYYCVS | RVEAEDVGV | NWYVDGVEVHNAKTKPREEQYNSTYRVVSVLTV | PSDEQLKSGTASVVCL |
| | | | | GKGDWGQGTL | YYCWQGTHF | LHQDWLNGKEYKCKVSNKALPAPIEKTISKAKG | LNNFYPREAKVQWKVD |
| | | | | VTVSS | PWTFGAGTR | QPREPQVYTLPPSREEMTKNQVSLTCLVKGFYP | NALQSGNSQESVTEQD |
| | | | | | LEIK | SDIAVEWESNGQPENNYKTTPPVLDSDGSFFLY | SKDSTYSLSSTLTLSK |
| | | | | | | SKLTVDKSRWQQGNVFSCSVMHEALHNHYTQKS | ADYEKHKVYACEVTHQ |
| | | | | | | LSLSPG | GLSSPVTKSFNRGEC |
| | (SEQ ID | | | 129 | 130 | 131 | 132 |
| | NOs:) | | | | | | |
| 4 | 8-G17-A | IgG3 | Kappa | EVQLVESGGG | DVVMTQTPL | MDPKGSLSWRILLFLSLAFELSYGEVQLVESGG | METDTLLLWVLLLWVP |
| | | | | LVKPGGSLKL | SLPVSLGDQ | GLVKPGGSLKLSCEASGITFSNYVMSWVRQTPE | GSTGDVVMTQTPLSLP |
| | | | | SCEASGITFS | ASISCRSSQ | KRLEWVATISDGGSYTYYPDNLKGRFTISRDNA | VSLGDQASISCRSSQS |
| | | | | NYVMSWVRQT | SLVHSNGNT | KNNLYLQMSHLKSEDTAMYYCVRDRYDYDGRVY | LVHSNGNTYLHWYLQK |
| | | | | PEKRLEWVAT | YLHWYLQKP | AMDYWGQGTSVTVSSATTTAPSVYPLVPGCSDT | PGQSPKLLIYKVSNRF |
| | | | | ISDGGSYTYY | GQSPKLLIY | SGSSVTLGCLVKGYFPEPVTVKWNYGALSSGVR | SGVPDRFSGSGSGTDF |
| | | | | PDNLKGRFTI | KVSNRFSGV | TVSSVLQSGFYSLSSLVTVPSSTWPSQTVICNV | TLKITRVEAEDLGVYF |
| | | | | SRDNAKNNLY | PDRFSGSGS | AHPASKTELIKRIEPRIPKPSTPPGSSCPPGNI | CSQSTHVPWTFGGGTK |
| | | | | LQMSHLKSED | GTDFTLKIT | LGGPSVFIFPPKPKDALMISLTPKVTCVVVDVS | LEIKRADAAPTVSIFP |
| | | | | TAMYYCVRDR | RVEAEDLGV | EDDPDVHVSWFVDNKEVHTAWTQPREAQYNSTF | PSSEQLTSGGASVVCF |
| | | | | YDYDGRVYAM | YFCSQSTHV | RVVSALPIQHQDWMRGKEFKCKVNNKALPAPIE | LNNFYPKDINVKWKID |
| | | | | DYWGQGTSVT | PWTFGGGTK | RTISKPKGRAQTPQVYTIPPPREQMSKKKVSLT | GSERQNGVLNSWTDQD |
| | | | | VSS | LEIK | CLVTNFFSEAISVEWERNGELEQDYKNTPPILD | SKDSTYSMSSTLTLTK |
| | | | | | | SDGTYFLYSKLTVDTDSWLQGEIFTCSVVHEAL | DEYERHNSYTCEATHK |
| | | | | | | HNHHTQKNLSRSPG | TSTSPIVKSFNRNEC |
| | (SEQ ID | | | 163 | 164 | 165 | 166 |
| | NOs:) | | | | | | |
| 5 | 6-N03-A | IgG1 | Kappa | EVQLVESGGG | DVVMTQTPL | MDPKGSLSWRILLFLSLAFELSYGEVQLVESGG | METDTLLLWVLLLWVP |
| | | | | LVKPGGSLKL | SLPVSLGDQ | GLVKPGGSLKLSCEASGITFSNYVMSWVRQTPE | GSTGDVVMTQTPLSLP |
| | | | | SCEASGITFS | ASISCRSSQ | KRLEWVATISDGGSYTYYPDNVKGRFTISRDNA | VSLGDQASISCRSSQS |
| | | | | NYVMSWVRQT | SLVHSNGDT | KNNLYLQMSHLKSEDTAMYYCVRDRYDYDGRVY | LVHSNGDTYLHWYLQK |
| | | | | PEKRLEWVAT | YLHWYLQKP | AMDYWGQGTSVTVSSAKTTPPSVYPLAPGSAAQ | PGQSPKLLIYKVSNRF |
| | | | | ISDGGSYTYY | GQSPKLLIY | TNSMVTLGCLVKGYFPEPVTVTWNSGSLSSGVH | SGVPDRFSGSGSGTDF |
| | | | | PDNVKGRFTI | KVSNRFSGV | TFPAVLQSDLYTLSSSVTVPSSTWPSETVTCNV | TLKITRVEAEDLGVYF |
| | | | | SRDNAKNNLY | PDRFSGSGS | AHPASSTKVDKKIVPRDCGCKPCICTVPEVSSV | CSQSTHVPWTFGGGTK |
| | | | | LQMSHLKSED | GTDFTLKIT | FIFPPKPKDVLTITLTPKVTCVVVDISKDDPEV | LEIKRADAAPTVSIFP |
| | | | | TAMYYCVRDR | RVEAEDLGV | QFSWFVDDVEVHTAQTQPREEQFNSTFRSVSEL | PSSEQLTSGGASVVCF |
| | | | | YDYDGRVYAM | YFCSQSTHV | PIMHQDWLNGKEFKCRVNSAAFPAPIEKTISKT | LNNFYPKDINVKWKID |
| | | | | DYWGQGTSVT | PWTFGGGTK | KGRPKAPQVYTIPPPKEQMAKDKVSLTCMITDF | GSERQNGVLNSWTDQD |
| | | | | VSS | LEIK | FPEDITVEWQWNGQPAENYKNTQPIMDTDGSYF | SKDSTYSMSSTLTLTK |
| | | | | | | VYSKLNVQKSNWEAGNTFTCSVLHEGLHNHHTE | DEYERHNSYTCEATHK |
| | | | | | | KSLSHSPG | TSTSPIVKSFNRNEC |
| | (SEQ ID | | | 197 | 198 | 199 | 200 |
| | NOs:) | | | | | | |
| 6 | R3- | IgG1 | Kappa | EVQLVESGGG | DIQMTQSPP | MDPKGSLSWRILLFLSLAFELSYGEVQLVESGG | METDTLLLWVLLLWVP |
| | P3-C11 | | | LVQPGRSLRL | SLSASVGDR | GLVQPGRSLRLACEASGFTFNNYAVHWVRQAPG | GSTGDIQMTQSPPSLS |
| | | | | ACEASGFTFN | VTITCRASQ | KGLEWVAVISYDGTNKYYSDSMKGRFTISRDNS | ASVGDRVTITCRASQS |
| | | | | NYAVHWVRQA | SISSYLNWY | KNTLYLQINSLRVEDTAVYFCARGNYVRRDSFD | ISSYLNWYQQKPGKAP |
| | | | | PGKGLEWVAV | QQKPGKAPK | IWGQGTMVTVSSASTKGPSVFPLAPSSKSTSGG | KLLIYGASSLQSGVPL |
| | | | | ISYDGTNKYY | LLIYGASSL | TAALGCLVKDYFPEPVTVSWNSGALTSGVHTFP | RFSGSGSGADFTLTIS |
| | | | | SDSMKGRFTI | QSGVPLRFS | AVLQSSGLYSLSSVVTVPSSSLGTQTYICNVNH | SLQPEDFATYYCQQSY |
| | | | | SRDNSKNTLY | GSGSGADFT | KPSNTKVDKKVEPKSCDKTHTCPPCPAPELLGG | ISPITFGQGTRLEIKR |
| | | | | LQINSLRVED | LTISSLQPE | PSVFLFPPKPKDTLMISRTPEVTCVVVDVSHED | TVAAPSVFIFPPSDEQ |
| | | | | TAVYFCARGN | DFATYYCQQ | PEVKFNWYVDGVEVHNAKTKPREEQYNSTYRVV | LKSGTASVVCLLNNFY |
| | | | | YVRRDSFDIW | SYISPITFG | SVLTVLHQDWLNGKEYKCKVSNKALPAPIEKTI | PREAKVQWKVDNALQS |
| | | | | GQGTMVTVSS | QGTRLEIK | SKAKGQPREPQVYTLPPSREEMTKNQVSLTCLV | GNSQESVTEQDSKDST |
| | | | | | | KGFYPSDIAVEWESNGQPENNYKTTPPVLDSDG | YSLSSTLTLSKADYEK |
| | | | | | | SFFLYSKLTVDKSRWQQGNVFSCSVMHEALHNH | HKVYACEVTHQGLSSP |
| | | | | | | YTQKSLSLSPG | VTKSFNRGEC |
| | (SEQ ID | | | 231 | 232 | 233 | 234 |
| | NOS:) | | | | | | |
| 7 | R4M- | IgG1 | Kappa | EVQLVESGGG | DIVMTQSPL | MDPKGSLSWRILLFLSLAFELSYGEVQLVESGG | METDTLLLWVLLLWVP |
| | P3-E06 | | | VVQPGGSLRL | SLPVTLGQP | GVVQPGGSLRLSCAASGFTFSNYGMHWVRQAPG | GSTGDIVMTQSPLSLP |
| | | | | SCAASGFTFS | ASISCRSSQ | KGLEWVATISYDGTKKYYPDSVRGRFTISRDNS | VTLGQPASISCRSSQS |
| | | | | NYGMHWVRQA | SLVHSDGNT | ENMLYLQMDSLRLEDTAVIYCAKDLVSGSRYGF | LVHSDGNTYLNWFQQR |
| | | | | PGKGLEWVAT | YLNWFQQRP | AFDSWGPGTLVTVSSASTKGPSVFPLAPSSKST | PGQSPRRLIYKVSNRD |
| | | | | ISYDGTKKYY | GQSPRRLIY | SGGTAALGCLVKDYFPEPVTVSWNSGALTSGVH | SGVPDRFSGTGSGTDF |
| | | | | PDSVRGRFTI | KVSNRDSGV | TFPAVLQSSGLYSLSSVVTVPSSSLGTQTYICN | TLKISRVEADDVGVYY |
| | | | | SRDNSENMLY | PDRFSGTGS | VNHKPSNTKVDKKVEPKSCDKTHTCPPCPAPEL | CMQGTHWPPIFGQGTR |
| | | | | LQMDSLRLED | GTDFTLKIS | LGGPSVFLFPPKPKDTLMISRTPEVTCVVVDVS | LEIKRTVAAPSVFIFP |
| | | | | TAVIYCAKDL | RVEADDVGV | HEDPEVKFNWYVDGVEVHNAKTKPREEQYNSTY | PSDEQLKSGTASVVCL |
| | | | | VSGSRYGFAF | YYCMQGTHW | RVVSVLTVLHQDWLNGKEYKCKVSNKALPAPIE | LNNFYPREAKVQWKVD |
| | | | | DSWGPGTLVT | PPIFGQGTR | KTISKAKGQPREPQVYTLPPSREEMTKNQVSLT | NALQSGNSQESVTEQD |
| | | | | VSS | LEIK | CLVKGFYPSDIAVEWESNGQPENNYKTTPPVLD | SKDSTYSLSSTLTLSK |
| | | | | | | SDGSFFLYSKLTVDKSRWQQGNVFSCSVMHEAL | ADYEKHKVYACEVTHQ |
| | | | | | | HNHYTQKSLSLSPG | GLSSPVTKSFNRGEC |
| | (SEQ ID | | | 265 | 266 | 267 | 268 |
| | NOs:) | | | | | | |
| 8 | R3- | IgG1 | Kappa | EVQLVETGGG | AIRMTQSPS | MDPKGSLSWRILLFLSLAFELSYGEVQLVETGG | METDTLLLWVLLLWVP |
| | P3-E09 | | | VVQPGRSLTL | SLSASVGDR | GVVQPGRSLTLSCGASEFTFSNYAMHWVRQAPG | GSTGAIRMTQSPSSLS |
| | | | | SCGASEFTFS | VTITCRASQ | KGLEWVAVISYDGTKKYYGDSVKGRFTISRENI | ASVGDRVTITCRASQG |
| | | | | NYAMHWVRQA | GIGSYLAWF | KNTLYLQMNSLRSEDTAVYYCARDVGKGVTATG | IGSYLAWFQQKPGEAP |
| | | | | PGKGLEWVAV | QQKPGEAPK | TFDIWGQGTMVTVSSASTKGPSVFPLAPSSKST | KSLIIDATRLKSGVPS |
| | | | | ISYDGTKKYY | SLIIDATRL | SGGTAALGCLVKDYFPEPVTVSWNSGALTSGVH | RFSGSGSGTEFALTIS |
| | | | | GDSVKGRFTI | KSGVPSRFS | TFPAVLQSSGLYSLSSVVTVPSSSLGTQTYICN | SLRPEDFATYYCQQYN |
| | | | | SRENIKNTLY | GSGSGTEFA | VNHKPSNTKVDKKVEPKSCDKTHTCPPCPAPEL | VSPLTFGGGTKVEIKR |
| | | | | LQMNSLRSED | LTISSLRPE | LGGPSVFLFPPKPKDTLMISRTPEVTCVVVDVS | TVAAPSVFIFPPSDEQ |
| | | | | TAVYYCARDV | DFATYYCQQ | HEDPEVKFNWYVDGVEVHNAKTKPREEQYNSTY | LKSGTASVVCLLNNFY |
| | | | | GKGVTATGTF | YNVSPLTFG | RVVSVLTVLHQDWLNGKEYKCKVSNKALPAPIE | PREAKVQWKVDNALQS |
| | | | | DIWGQGTMVT | GGTKVEIK | KTISKAKGQPREPQVYTLPPSREEMTKNQVSLT | GNSQESVTEQDSKDST |
| | | | | VSS | | CLVKGFYPSDIAVEWESNGQPENNYKTTPPVLD | YSLSSTLTLSKADYEK |
| | | | | | | SDGSFFLYSKLTVDKSRWQQGNVFSCSVMHEAL | HKVYACEVTHQGLSSP |
| | | | | | | HNHYTQKSLSLSPG | VTKSFNRGEC |
| | (SEQ ID | | | 299 | 300 | 301 | 302 |
| | NOs:) | | | | | | |
| 9 | R3- | IgG1 | Kappa | EVQLVESGGG | DVVMTQSPL | MDPKGSLSWRILLFLSLAFELSYGEVQLVESGG | METDTLLLWVLLLWVP |
| | P1-C02 | | | VVQPGGSLRI | SLPVTLGQP | GVVQPGGSLRISCVASEFPFSDFGMHWVRQAPG | GSTGDVVMTQSPLSLP |
| | | | | SCVASEFPFS | ASISCRSSQ | KGLEWVAFVRSHGSGKYYADSVKGRFTISGDKS | VTLGQPASISCRSSQS |
| | | | | DFGMHWVRQA | SLVYSDGNT | KSTLYLQMESLRPDDAAVYYCTTLADVWGQGTT | LVYSDGNTYLNWFQQR |
| | | | | PGKGLEWVAF | YLNWFQQRP | VTVSSASTKGPSVFPLAPSSKSTSGGTAALGCL | PGQSPRRLIYKVSNRD |
| | | | | VRSHGSGKYY | GQSPRRLIY | VKDYFPEPVTVSWNSGALTSGVHTFPAVLQSSG | SGVPDRFSGSGSGTDF |
| | | | | ADSVKGRFTI | KVSNRDSGV | LYSLSSVVTVPSSSLGTQTYICNVNHKPSNTKV | TLKISRVEAEDVGVYY |
| | | | | SGDKSKSTLY | PDRFSGSGS | DKKVEPKSCDKTHTCPPCPAPELLGGPSVFLFP | CMQGTHWPWTFGQGTK |
| | | | | LQMESLRPDD | GTDFTLKIS | PKPKDTLMISRTPEVTCVVVDVSHEDPEVKFNW | VEIKRTVAAPSVFIFP |
| | | | | AAVYYCTTLA | RVEAEDVGV | YVDGVEVHNAKTKPREEQYNSTYRVVSVLTVLH | PSDEQLKSGTASVVCL |
| | | | | DVWGQGTTVT | YYCMQGTHW | QDWLNGKEYKCKVSNKALPAPIEKTISKAKGQP | LNNFYPREAKVQWKVD |
| | | | | VSS | PWTFGQGTK | REPQVYTLPPSREEMTKNQVSLTCLVKGFYPSD | NALQSGNSQESVTEQD |
| | | | | | VEIK | IAVEWESNGQPENNYKTTPPVLDSDGSFFLYSK | SKDSTYSLSSTLTLSK |
| | | | | | | LTVDKSRWQQGNVFSCSVMHEALHNHYTQKSLS | ADYEKHKVYACEVTHQ |
| | | | | | | LSPG | GLSSPVTKSFNRGEC |
| | (SEQ ID | | | 333 | 334 | 335 | 336 |
| | NOs:) | | | | | | |
| 10 | R3- | IgG1 | Kappa | EVQLVESGGG | DVVMTQSPL | MDPKGSLSWRILLFLSLAFELSYGEVQLVESGG | METDTLLLWVLLLWVP |
| | P3-A12 | | | LVQPGRSLRL | SLPVTPGEP | GLVQPGRSLRLSCGVSGFTFSSFGMHWVRQAPG | GSTGDVVMTQSPLSLP |
| | | | | SCGVSGFTFS | ASISCRSSQ | KGLEWVAVISNDALHKNYGDSVKGRFTVTRDNS | VTPGEPASISCRSSQS |
| | | | | SFGMHWVRQA | SLLYSNGYN | RNTLFLQMTSLRPEDTAVYYCAKDVVRGHTSGS | LLYSNGYNYLDWYLQK |
| | | | | PGKGLEWVAV | YLDWYLQKP | LGSWGQGTLVTVSSASTKGPSVFPLAPSSKSTS | PGQSPQLLIYLGSNRA |
| | | | | ISNDALHKNY | GQSPQLLIY | GGTAALGCLVKDYFPEPVTVSWNSGALTSGVHT | SGVPDRFSGSGSGTDF |
| | | | | GDSVKGRFTV | LGSNRASGV | FPAVLQSSGLYSLSSVVTVPSSSLGTQTYICNV | TLKISRVEAEDVGVYY |
| | | | | TRDNSRNTLF | PDRFSGSGS | NHKPSNTKVDKKVEPKSCDKTHTCPPCPAPELL | CMQALQTPLTFGGGTK |
| | | | | LQMTSLRPED | GTDFTLKIS | GGPSVFLFPPKPKDTLMISRTPEVTCVVVDVSH | LEIKRTVAAPSVFIFP |
| | | | | TAVYYCAKDV | RVEAEDVGV | EDPEVKFNWYVDGVEVHNAKTKPREEQYNSTYR | PSDEQLKSGTASVVCL |
| | | | | VRGHTSGSLG | YYCMQALQT | VVSVLTVLHQDWLNGKEYKCKVSNKALPAPIEK | LNNFYPREAKVQWKVD |
| | | | | SWGQGTLVTV | PLTFGGGTK | TISKAKGQPREPQVYTLPPSREEMTKNQVSLTC | NALQSGNSQESVTEQD |
| | | | | SS | LEIK | LVKGFYPSDIAVEWESNGQPENNYKTTPPVLDS | SKDSTYSLSSTLTLSK |
| | | | | | | DGSFFLYSKLTVDKSRWQQGNVFSCSVMHEALH | ADYEKHKVYACEVTHQ |
| | | | | | | NHYTQKSLSLSPG | GLSSPVTKSFNRGEC |
| | (SEQ ID | | | 367 | 368 | 369 | 370 |
| | NOs:) | | | | | | |
| 11 | R4M- | IgG1 | Lamba | EVQLVESGGG | QSVLTQPPS | MDPKGSLSWRILLFLSLAFELSYGEVQLVESGG | METDTLLLWVLLLWVP |
| | P3-A12 | | | VVQPGGSLRL | ASGTPGQRV | GVVQPGGSLRLSCAASGFTFSDHYMSWIRQAPG | GSTGQSVLTQPPSASG |
| | | | | SCAASGFTFS | TISCSGSSS | KGLEWVSYLSGSGTSIYYADSVKGRFTISRDNA | TPGQRVTISCSGSSSN |
| | | | | DHYMSWIRQA | NIGSNYVYW | KKSLYLQMNSLRAEDTAVYYCARVGPAAGNAFD | IGSNYVYWYQQLPGTA |
| | | | | PGKGLEWVSY | YQQLPGTAP | IWGQGTMVTVSSASTKGPSVFPLAPSSKSTSGG | PKLLIYRNNQRPSGVP |
| | | | 3 | LSGSGTSIYY | KLLIYRNNQ | TAALGCLVKDYFPEPVTVSWNSGALTSGVHTFP | DRFSGSKSGTSASLAI |
| | | | | ADSVKGRFTI | RPSGVPDRF | AVLQSSGLYSLSSVVTVPSSSLGTQTYICNVNH | TGLQAEDEADYYCQSY |
| | | | | SRDNAKKSLY | SGSKSGTSA | KPSNTKVDKKVEPKSCDKTHTCPPCPAPELLGG | DSSLSGVVFGGGTKLT |
| | | | | LQMNSLRAED | SLAITGLQA | PSVFLFPPKPKDTLMISRTPEVTCVVVDVSHED | VLRTVAAPSVFIFPPS |
| | | | | TAVYYCARVG | EDEADYYCQ | PEVKFNWYVDGVEVHNAKTKPREEQYNSTYRVV | DEQLKSGTASVVCLLN |
| | | | | PAAGNAFDIW | SYDSSLSGV | SVLTVLHQDWLNGKEYKCKVSNKALPAPIEKTI | NFYPREAKVQWKVDNA |
| | | | | GQGTMVTVSS | VFGGGTKLT | SKAKGQPREPQVYTLPPSREEMTKNQVSLTCLV | LQSGNSQESVTEQDSK |
| | | | | | VL | KGFYPSDIAVEWESNGQPENNYKTTPPVLDSDG | DSTYSLSSTLTLSKAD |
| | | | | | | SFFLYSKLTVDKSRWQQGNVFSCSVMHEALHNH | YEKHKVYACEVTHQGL |
| | | | | | | YTQKSLSLSPG | SSPVTKSFNRGEC |
| | (SEQ ID | | | 401 | 402 | 403 | 404 |
| | NOs:) | | | | | | |
| 12 | R4M- | IgG1 | Lamba | EVQLVETGGG | QAGLTQPPS | MDPKGSLSWRILLFLSLAFELSYGEVQLVETGG | METDTLLLWVLLLWVP |
| | P1-A10 | | | VVQPGRSLRL | VSKGLRQTA | GVVQPGRSLRLSCAASGFTFSSYAMHWVRQAPG | GSTGQAGLTQPPSVSK |
| | | | | SCAASGFTFS | TLTCTGNSN | KGLEWVAVISYDGSNKYYADSVKGRFTISRDNS | GLRQTATLTCTGNSNN |
| | | | 3 | SYAMHWVRQA | NVGYQGAVW | KNTLYLQMNSLRAEDTAVYYCARDRGWADAFDI | VGYQGAVWLLQYQGHP |
| | | | | PGKGLEWVAV | LLQYQGHPP | WGQGTMVTVSSASTKGPSVFPLAPSSKSTSGGT | PKVLSNRNNDRPSGIS |
| | | | | ISYDGSNKYY | KVLSNRNND | AALGCLVKDYFPEPVTVSWNSGALTSGVHTFPA | ERLSASRSGNTASLTI |
| | | | | ADSVKGRFTI | RPSGISERL | VLQSSGLYSLSSVVTVPSSSLGTQTYICNVNHK | TGLQPEDEGDYFCAAW |
| | | | | SRDNSKNTLY | SASRSGNTA | PSNTKVDKKVEPKSCDKTHTCPPCPAPELLGGP | DGSLRGWVFGGGTKLT |
| | | | | LQMNSLRAED | SLTITGLQP | SVFLFPPKPKDTLMISRTPEVTCVVVDVSHEDP | VLRTVAAPSVFIFPPS |
| | | | | TAVYYCARDR | EDEGDYFCA | EVKFNWYVDGVEVHNAKTKPREEQYNSTYRVVS | DEQLKSGTASVVCLLN |
| | | | | GWADAFDIWG | AWDGSLRGW | VLTVLHQDWLNGKEYKCKVSNKALPAPIEKTIS | NFYPREAKVQWKVDNA |
| | | | | QGTMVTVSS | VFGGGTKLT | KAKGQPREPQVYTLPPSREEMTKNQVSLTCLVK | LQSGNSQESVTEQDSK |
| | | | | | VL | GFYPSDIAVEWESNGQPENNYKTTPPVLDSDGS | DSTYSLSSTLTLSKAD |
| | | | | | | FFLYSKLTVDKSRWQQGNVFSCSVMHEALHNHY | YEKHKVYACEVTHQGL |
| | | | | | | TQKSLSLSPG | SSPVTKSFNRGEC |
| | (SEQ ID | | | 435 | 436 | 437 | 438 |
| | NOs:) | | | | | | |

TABLE 4
Anti-Sdc2 Antibody Kabat CDR Amino Acid Sequences

| | Protein | HC Kabat | | HC Kabat | | LC Kabat | |
|---|---|---|---|---|---|---|---|
| # | Name | CDR1 | HC Kabat CDR2 | CDR3 | LC Kabat CDR1 | CDR2 | LC Kabat CDR3 |
| 1 | 20-H19-AB | IYAMH | RIGSKSSYYATYYADSVKD | GKGD | KSSQSLLAGDGKTYLN | LVSKLDS | WQGTHFPWT |
| | (SEQ ID NOS:) | 31 | 32 | 33 | 34 | 35 | 36 |
| 2 | TP-43327F | IYAMH | RIGSKSSYYATYYADSVKG | GKGD | RSSQSLLAGDGKTYLN | LVSKLDS | WOGTHFPWT |
| | (SEQ ID NOs:) | 65 | 66 | 67 | 68 | 69 | 70 |
| 3 | TP-43329F | IYAMH | RIGSKSSYYATYYADSVKG | GKGD | RSSQSLLAGDGKTYLN | LVSKLDS | WQGTHFPWT |
| | (SEQ ID NOs:) | 99 | 100 | 101 | 102 | 103 | 104 |
| 4 | 8-G17-A | NYVMS | TISDGGSYTYYPDNLKG | DRYDYDGRV | RSSQSLVHSNGNTYLH | KVSNRFS | SQSTHVPWT |
| | | | | YAMDY | | | |
| | (SEQ ID NOs:) | 133 | 134 | 135 | 136 | 137 | 138 |
| 5 | 6-N03-A | NYVMS | TISDGGSYTYYPDNVKG | DRYDYDGRV | RSSQSLVHSNGDTYLH | KVSNRFS | SQSTHVPWT |
| | | | | YAMDY | | | |
| | (SEQ ID NOS:) | 167 | 168 | 169 | 170 | 171 | 172 |
| 6 | R3-P3-C11 | NYAVH | VISYDGTNKYYSDSMKG | GNYVRRDSF | RASQSISSYLN | GASSLOS | QQSYISPIT |
| | | | | DI | | | |
| | (SEQ ID NOs:) | 201 | 202 | 203 | 204 | 205 | 206 |
| 7 | R4M-P3-E06 | NYGMH | TISYDGTKKYYPDSVRG | DLVSGSRYG | RSSQSLVHSDGNTYLN | KVSNRDS | MQGTHWPPI |
| | | | | FAFDS | | | |
| | (SEQ ID NOS:) | 235 | 236 | 237 | 238 | 239 | 240 |
| 8 | R3-P3-E09 | NYAMH | VISYDGTKKYYGDSVKG | DVGKGVTAT | RASQGIGSYLA | DATRLKS | QQYNVSPLT |
| | | | | GTFDI | | | |
| | (SEQ ID NOs:) | 269 | 270 | 271 | 272 | 273 | 274 |
| 9 | R3-P1-C02 | DFGMH | FVRSHGSGKYYADSVKG | LADV | RSSQSLVYSDGNTYLN | KVSNRDS | MQGTHWPWT |
| | (SEQ ID NOs:) | 303 | 304 | 305 | 306 | 307 | 308 |
| 10 | R3-P3-A12 | SFGMH | VISNDALHKNYGDSVKG | DVVRGHTSG | RSSQSLLYSNGYNYLD | LGSNRAS | MQALQTPLT |
| | | | | SLGS | | | |
| | (SEQ ID NOS:) | 337 | 338 | 339 | 340 | 341 | 342 |
| 11 | R4M-P3-A12 | DHYMS | YLSGSGTSIYYADSVKG | VGPAAGNAF | SGSSSNIGSNYVY | RNNQRPS | QSYDSSLSGVV |
| | | | | DI | | | |
| | (SEQ ID NOS:) | 371 | 372 | 373 | 374 | 375 | 376 |
| 12 | R4M-P1-A10 | SYAMH | VISYDGSNKYYADSVKG | DRGWADAFD | N/A | N/A | N/A |
| | | | | I | | | |
| | (SEQ ID NOs:) | 405 | 406 | 407 | 408 | 409 | 410 |

TABLE 5
Anti-Sdc2 Antibody Chothia CDR Amino Acid Sequences

| | | | | | | LC | |
|---|---|---|---|---|---|---|---|
| | Protein | HC Chothia | HC Chothia | HC Chothia | LC Chothia | Chothia | LC Chothia |
| # | Name | CDR1 | CDR2 | CDR3 | CDR1 | CDR2 | CDR3 |
| 1 | 20-H19-AB | GFTFNIY | GSKSSYYA | GKGD | KSSQSLLAGDGKTYLN | LVSKLDS | WQGTHFPWT |
| | (SEQ ID NOs:) | 37 | 38 | 39 | 40 | 41 | 42 |
| 2 | TP-43327F | GFTFNIY | GSKSSYYA | GKGD | RSSQSLLAGDGKTYLN | LVSKLDS | WQGTHFPWT |
| | (SEQ ID NOS:) | 71 | 72 | 73 | 74 | 75 | 76 |
| 3 | TP-43329F | GFTFNIY | GSKSSYYA | GKGD | RSSQSLLAGDGKTYLN | LVSKLDS | WOGTHFPWT |
| | (SEQ ID NOs:) | 105 | 106 | 107 | 108 | 109 | 110 |
| 4 | 8-G17-A | GITFSNY | SDGGSY | DRYDYDGRVYAMDY | RSSQSLVHSNGNTYLH | KVSNRFS | SQSTHVPWT |
| | (SEQ ID NOs:) | 139 | 140 | 141 | 142 | 143 | 144 |
| 5 | 6-N03-A | GITFSNY | SDGGSY | DRYDYDGRVYAMDY | RSSQSLVHSNGDTYLH | KVSNRFS | SQSTHVPWT |
| | (SEQ ID NOs:) | 173 | 174 | 175 | 176 | 177 | 178 |
| 6 | R3-P3-C11 | GFTFNNY | SYDGTN | GNYVRRDSFDI | RASQSISSYLN | GASSLOS | QQSYISPIT |
| | (SEQ ID NOS:) | 207 | 208 | 209 | 210 | 211 | 212 |
| 7 | R4M-P3-E06 | GFTFSNY | SYDGTK | DLVSGSRYGFAFDS | RSSQSLVHSDGNTYLN | KVSNRDS | MQGTHWPPI |
| | (SEQ ID NOs:) | 241 | 242 | 243 | 244 | 245 | 246 |
| 8 | R3-P3-E09 | EFTFSNY | SYDGTK | DVGKGVTATGTFDI | RASQGIGSYLA | DATRLKS | QQYNVSPLT |
| | (SEQ ID NOs:) | 275 | 276 | 277 | 278 | 279 | 280 |
| 9 | R3-P1-C02 | EFPFSDF | RSHGSG | LADV | RSSQSLVYSDGNTYLN | KVSNRDS | MQGTHWPWT |
| | (SEQ ID NOs:) | 309 | 310 | 311 | 312 | 313 | 314 |
| 10 | R3-P3-A12 | GFTFSSF | SNDALH | DVVRGHTSGSLGS | RSSQSLLYSNGYNYLD | LGSNRAS | MQALQTPLT |
| | (SEQ ID NOs:) | 343 | 344 | 345 | 346 | 347 | 348 |
| 11 | R4M-P3-A12 | GFTFSDH | SGSGTS | VGPAAGNAFDI | SGSSSNIGSNYVY | RNNQRPS | QSYDSSLSGVV |
| | (SEQ ID NOs:) | 377 | 378 | 379 | 380 | 381 | 382 |
| 12 | R4M-P1-A10 | GFTFSSY | SYDGSN | DRGWADAFDI | SNNVGYQG | RNN | AAWDGSLRGWV |
| | (SEQ ID NOS:) | 411 | 412 | 413 | 414 | 415 | 416 |

TABLE 6
Anti-Sdc2 Antibody AbM CDR Amino Acid Sequences

| | | HC AbM | HC AbM | HC AbM | LC AbM | LC AbM | LC AbM |
|---|---|---|---|---|---|---|---|
| # | Protein Name | CDR1 | CDR2 | CDR3 | CDR1 | CDR2 | CDR3 |
| 1 | 20-H19-AB | GFTFNIYAMH | RIGSKSSYYATY | GKGD | KSSQSLLAGDGKTYLN | LVSKLDS | WQGTHFPWT |
| | (SEQ ID NOS:) | 43 | 44 | 45 | 46 | 47 | 48 |
| 2 | TP-43327F | GFTFNIYAMH | RIGSKSSYYATY | GKGD | RSSQSLLAGDGKTYLN | LVSKLDS | WQGTHFPWT |
| | (SEQ ID NOs:) | 77 | 78 | 79 | 80 | 81 | 82 |
| 3 | TP-43329F | GFTFNIYAMH | RIGSKSSYYATY | GKGD | RSSQSLLAGDGKTYLN | LVSKLDS | WQGTHFPWT |
| | (SEQ ID NOS:) | 111 | 112 | 113 | 114 | 115 | 116 |
| 4 | 8-G17-A | GITFSNYVMS | TISDGGSYTY | DRYDYDGRVYAMDY | RSSQSLVHSNGNTYLH | KVSNRFS | SQSTHVPWT |
| | (SEQ ID NOs:) | 145 | 146 | 147 | 148 | 149 | 150 |
| 5 | 6-NO3-A | GITFSNYVMS | TISDGGSYTY | DRYDYDGRVYAMDY | RSSQSLVHSNGDTYLH | KVSNRFS | SQSTHVPWT |
| | (SEQ ID NOS:) | 179 | 180 | 181 | 182 | 183 | 184 |
| 6 | R3-P3-C11 | GFTFNNYAVH | VISYDGTNKY | GNYVRRDSFDI | RASQSISSYLN | GASSLOS | QQSYISPIT |
| | (SEQ ID NOs:) | 213 | 214 | 215 | 216 | 217 | 218 |
| 7 | R4M-P3-E06 | GFTFSNYGMH | TISYDGTKKY | DLVSGSRYGFAFDS | RSSQSLVHSDGNTYLN | KVSNRDS | MQGTHWPPI |
| | (SEQ ID NOs:) | 247 | 248 | 249 | 250 | 251 | 252 |
| 8 | R3-P3-E09 | EFTFSNYAMH | VISYDGTKKY | DVGKGVTATGTFDI | RASQGIGSYLA | DATRLKS | QQYNVSPLT |
| | (SEQ ID NOs:) | 281 | 282 | 283 | 284 | 285 | 286 |
| 9 | R3-P1-C02 | EFPFSDFGMH | FVRSHGSGKY | LADV | RSSQSLVYS | MQGTHWPW | N/A |
| | | | | | DGNTYLN | T | |
| | (SEQ ID NOs:) | 315 | 316 | 317 | 318 | 319 | 320 |
| 10 | R3-P3-A12 | GFTFSSFGMH | VISNDALHKN | DVVRGHTSGSLGS | RSSQSLLYSNGYNYLD | LGSNRAS | MQALQTPLT |
| | (SEQ ID NOS:) | 349 | 350 | 351 | 352 | 353 | 354 |
| 11 | R4M-P3-A12 | GFTFSDHYMS | YLSGSGTSIY | VGPAAGNAFDI | SGSSSNIGSNYVY | RNNQRPS | QSYDSSLSGVV |
| | (SEQ ID NOs:) | 383 | 384 | 385 | 386 | 387 | 388 |
| 12 | R4M-P1-A10 | GFTFSSYAMH | VISYDGSNKY | DRGWADAFDI | SNNVGYQG | RNN | AAWDGSLRGWV |
| | (SEQ ID NOs:) | 417 | 418 | 419 | 420 | 421 | 422 |

TABLE 7
Anti-Sdc2 Antibody Contact CDR Amino Acid Sequences

| | | HC | | | | | |
|---|---|---|---|---|---|---|---|
| | Protein | Contact | HC Contact | HC Contact | LC Contact | LC Contact | LC Contact |
| # | Name | CDR1 | CDR2 | CDR3 | CDR1 | CDR2 | CDR3 |
| 1 | 20-H19-AB | NIYAMH | WIARIGSKSSYYATY | VSGKG | LAGDGKTYLNWL | RLIYLVSKLD | WQGTHFPW |
| | (SEQ ID NOs:) | 49 | 50 | 51 | 52 | 53 | 54 |
| 2 | TP-43327F | NIYAMH | WVARIGSKSSYYATY | VSGKG | LAGDGKTYLNWF | RLIYLVSKLD | WQGTHFPW |
| | (SEQ ID NOS:) | 83 | 84 | 85 | 86 | 87 | 88 |
| 3 | TP-43329F | NIYAMH | WVARIGSKSSYYATY | VSGKG | LAGDGKTYLNWF | RLIYLVSKLD | WQGTHFPW |
| | (SEQ ID NOs:) | 117 | 118 | 119 | 120 | 121 | 122 |
| 4 | 8-G17-A | SNYVMS | WVATISDGGSYTY | VRDRYDYDGRVYAMD | VHSNGNTYLHWY | LLIYKVSNRF | SQSTHVPW |
| | (SEQ ID NOs:) | 151 | 152 | 153 | 154 | 155 | 156 |
| 5 | 6-N03-A | SNYVMS | WVATISDGGSYTY | VRDRYDYDGRVYAMD | VHSNGDTYLHWY | LLIYKVSNRF | SQSTHVPW |
| | (SEQ ID NOs:) | 185 | 186 | 187 | 188 | 189 | 190 |
| 6 | R3-P3-C11 | NNYAVH | WVAVISYDGTNKY | ARGNYVRRDSFD | SSYLNWY | LLIYGASSLQ | QQSYISPI |
| | (SEQ ID NOs:) | 219 | 220 | 221 | 222 | 223 | 224 |
| 7 | R4M-P3-E06 | SNYGMH | WVATISYDGTKKY | AKDLVSGSRYGFAFD | VHSDGNTYLNWF | RLIYKVSNRD | MQGTHWPP |
| | (SEQ ID NOs:) | 253 | 254 | 255 | 256 | 257 | 258 |
| 8 | R3-P3-E09 | SNYAMH | WVAVISYDGTKKY | ARDVGKGVTATGTFD | GSYLAWF | SLIIDATRLK | QQYNVSPL |
| | (SEQ ID NOs:) | 287 | 288 | 289 | 290 | 291 | 292 |
| 9 | R3-P1-C02 | SDFGMH | WVAFVRSHGSGKY | TTLAD | VYSDGNTYLNWF | RLIYKVSNRD | MQGTHWPW |
| | (SEQ ID NOS:) | 321 | 322 | 323 | 324 | 325 | 326 |
| 10 | R3-P3-A12 | SSFGMH | WVAVISNDALHKN | AKDVVRGHTSGSLG | LYSNGYNYLDWY | LLIYLGSNRA | MQALQTPL |
| | (SEQ ID NOs:) | 355 | 356 | 357 | 358 | 359 | 360 |
| 11 | R4M-P3-A12 | SDHYMS | WVSYLSGSGTSIY | ARVGPAAGNAFD | IGSNYVYWY | LLIYRNNQRP | QSYDSSLSGV |
| | (SEQ ID NOs:) | 389 | 390 | 391 | 392 | 393 | 394 |
| 12 | R4M-P1-A10 | SSYAMH | WVAVISYDGSNKY | ARDRGWADAFD | SNNVGYQG | RNN | AAWDGSLRGWV |
| | (SEQ ID NOs:) | 423 | 424 | 425 | 426 | 427 | 428 |

TABLE 8
Anti-Sdc2 Antibody IMGT CDR Amino Acid Sequences

| | | HC IMGT | HC IMGT | | | LC IMGT | |
|---|---|---|---|---|---|---|---|
| # | Protein Name | CDR1 | CDR2 | HC IMGT CDR3 | LC IMGT CDR1 | CDR2 | LC IMGT CDR3 |
| 1 | 20-H19-AB | GFTFNIYA | IGSKSSYYAT | VSGKGD | QSLLAGDGKTY | LV | WQGTHFPWT |
| | (SEQ ID NOS:) | 55 | 56 | 57 | 58 | 59 | 60 |
| 2 | TP-43327F | GFTFNIYA | IGSKSSYYAT | VSGKGD | QSLLAGDGKTY | LV | WQGTHFPWT |
| | (SEQ ID NOs:) | 89 | 90 | 91 | 92 | 93 | 94 |
| 3 | TP-43329F | GFTFNIYA | IGSKSSYYAT | VSGKGD | QSLLAGDGKTY | LV | WQGTHFPWT |
| | (SEQ ID NOs:) | 123 | 124 | 125 | 126 | 127 | 128 |
| 4 | 8-G17-A | GITFSNYV | ISDGGSYT | VRDRYDYDGRVYAMDY | QSLVHSNGNTY | KV | SQSTHVPWT |
| | (SEQ ID NOS:) | 157 | 158 | 159 | 160 | 161 | 162 |
| 5 | 6-N03-A | GITFSNYV | ISDGGSYT | VRDRYDYDGRVYAMDY | QSLVHSNGDTY | KV | SQSTHVPWT |
| | (SEQ ID NOS:) | 191 | 192 | 193 | 194 | 195 | 196 |
| 6 | R3-P3-C11 | GFTFNNYA | ISYDGTNK | ARGNYVRRDSFDI | QSISSY | GA | QQSYISPIT |
| | (SEQ ID NOs:) | 225 | 226 | 227 | 228 | 229 | 230 |
| 7 | R4M-P3-E06 | GFTFSNYG | ISYDGTKK | AKDLVSGSRYGFAFDS | QSLVHSDGNTY | KV | MQGTHWPPI |
| | (SEQ ID NOS:) | 259 | 260 | 261 | 262 | 263 | 264 |
| 8 | R3-P3-E09 | EFTFSNYA | ISYDGTKK | ARDVGKGVTATGTFDI | QGIGSY | DA | QQYNVSPLT |
| | (SEQ ID NOS:) | 293 | 294 | 295 | 296 | 297 | 298 |
| 9 | R3-P1-C02 | EFPFSDFG | VRSHGSGK | TTLADV | QSLVYSDGNTY | KV | MQGTHWPWT |
| | (SEQ ID NOs:) | 327 | 328 | 329 | 330 | 331 | 332 |
| 10 | R3-P3-A12 | GFTFSSFG | ISNDALHK | AKDVVRGHTSGSLGS | QSLLYSNGYNY | LG | MQALQTPLT |
| | (SEQ ID NOs:) | 361 | 362 | 363 | 364 | 365 | 366 |
| 11 | R4M-P3-A12 | GFTFSDHY | LSGSGTSI | ARVGPAAGNAFDI | SSNIGSNY | RN | QSYDSSLSGVV |
| | (SEQ ID NOS:) | 395 | 396 | 397 | 398 | 399 | 400 |
| 12 | R4M-P1-A10 | GFTFSSYA | ISYDGSNK | ARDRGWADAFDI | SNNVGYQG | RNN | AAWDGSLRGWV |
| | (SEQ ID NOs:) | 429 | 430 | 431 | 432 | 433 | 434 |

### 7.3 Example 3: Characterization of Human and Humanized Anti-Sdc2 Antibodies

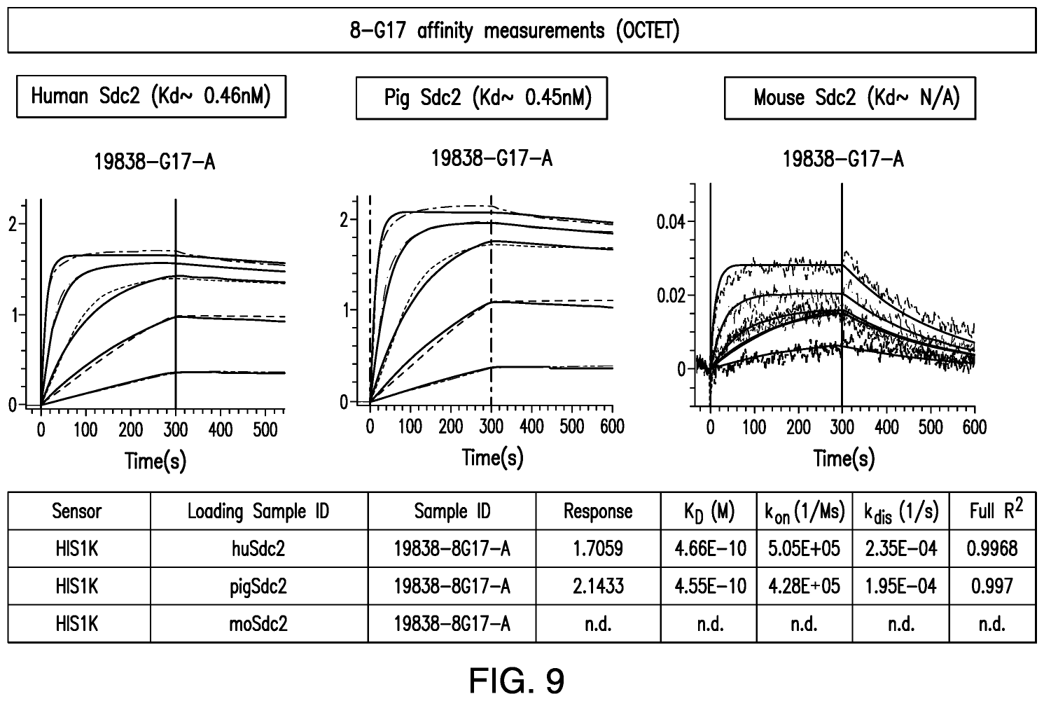

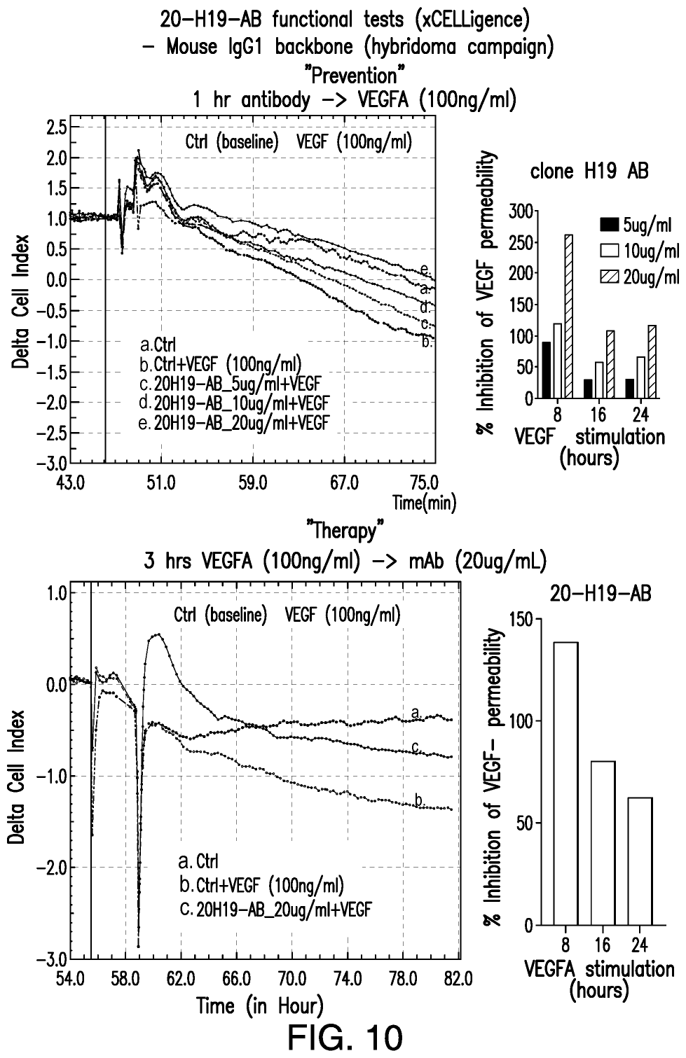

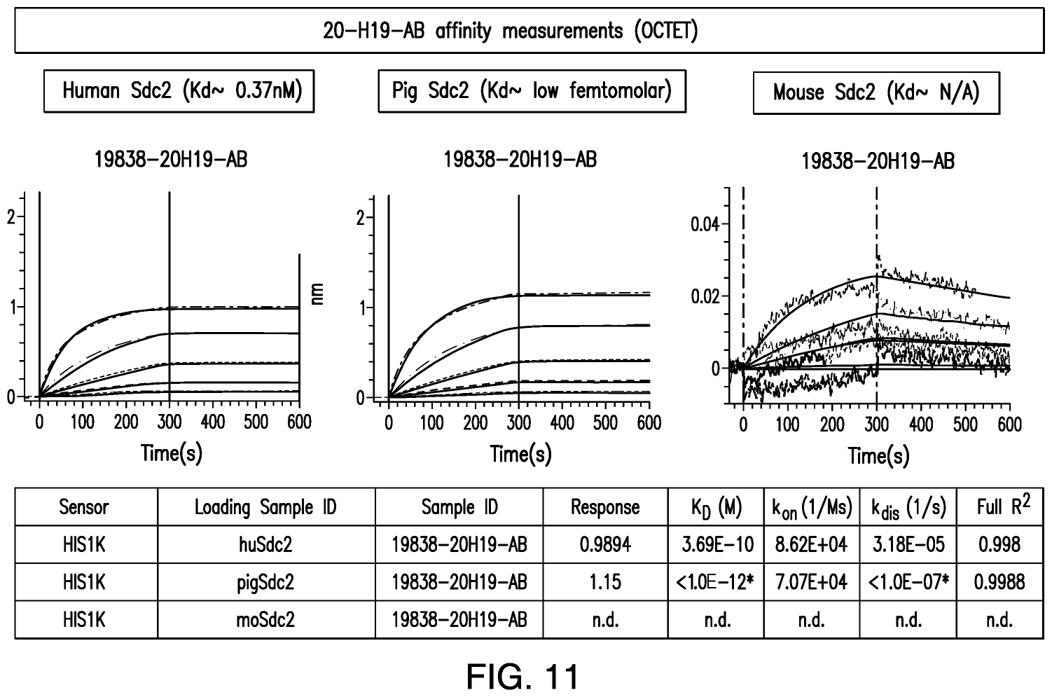

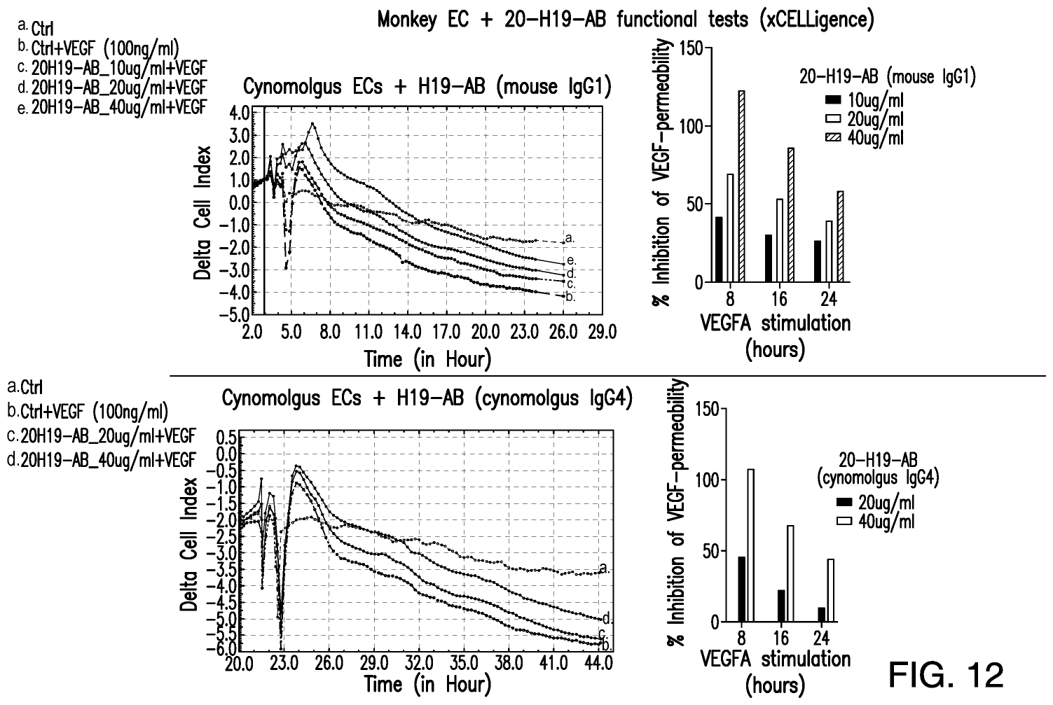

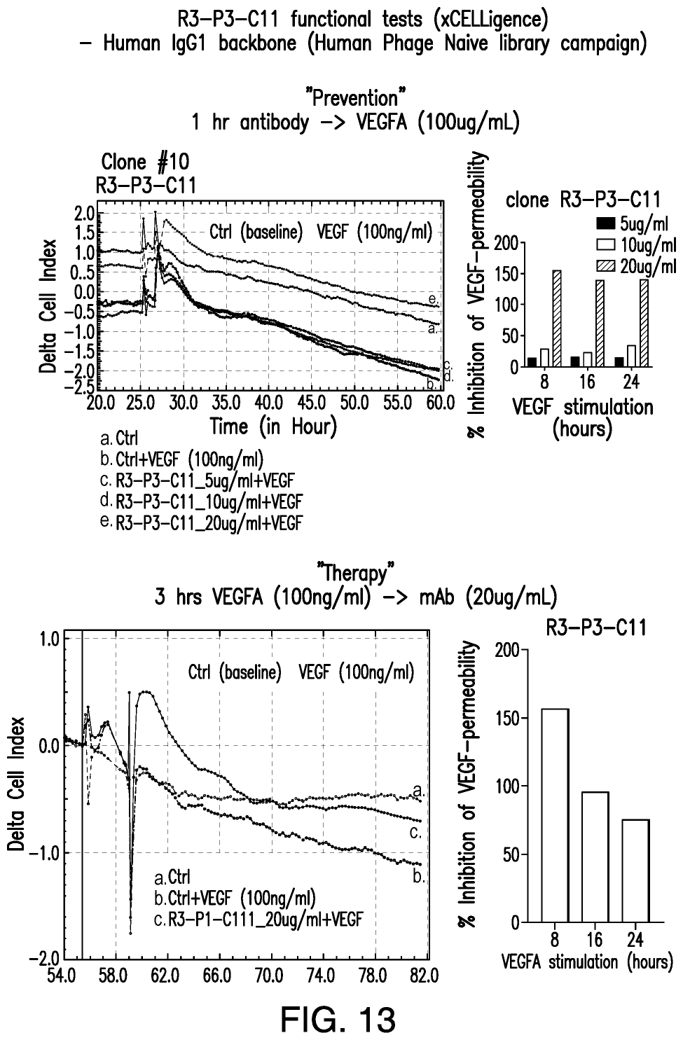

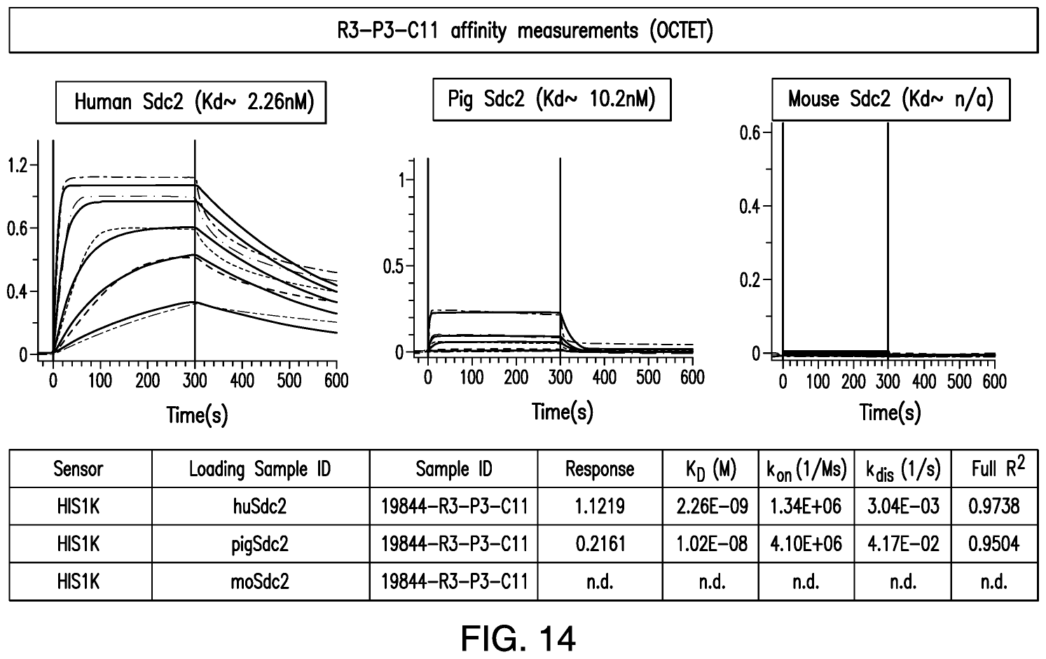

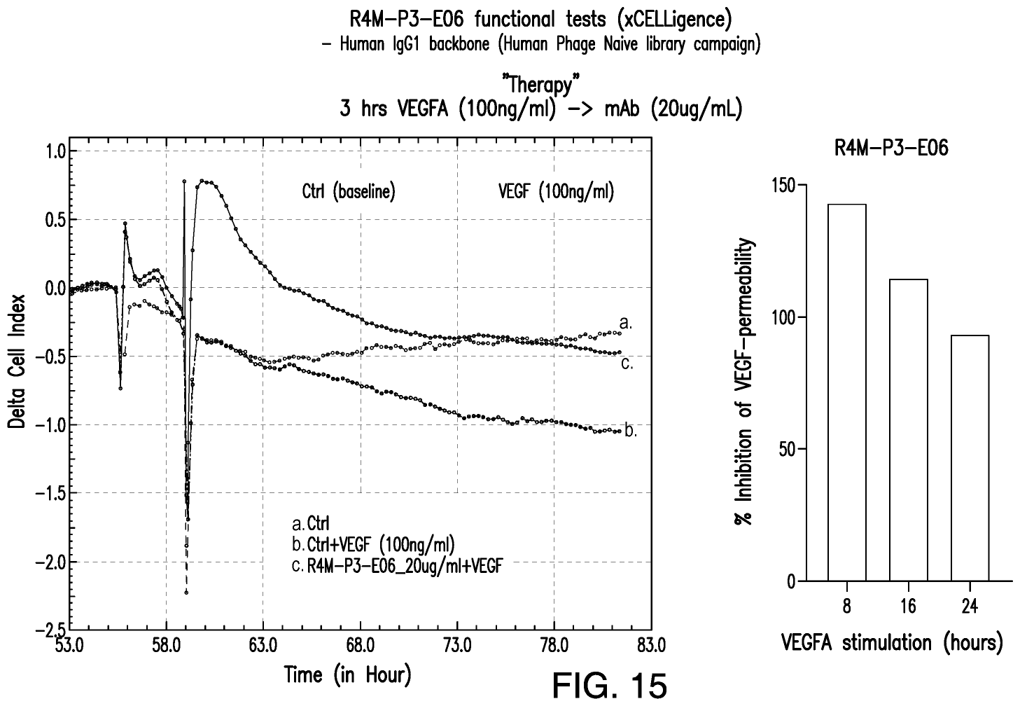

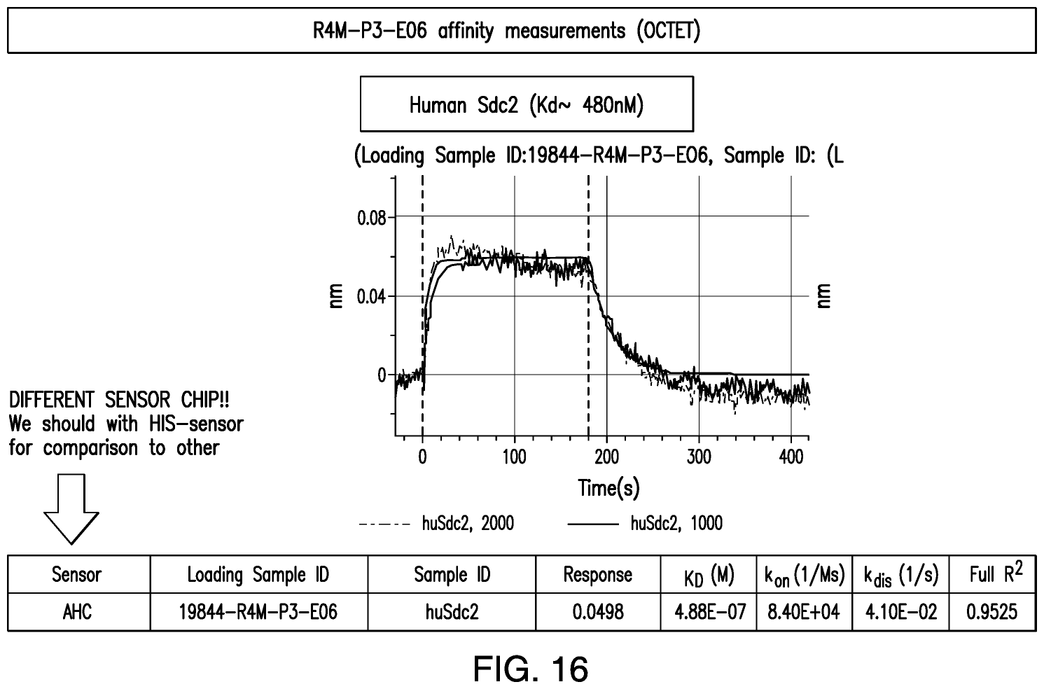

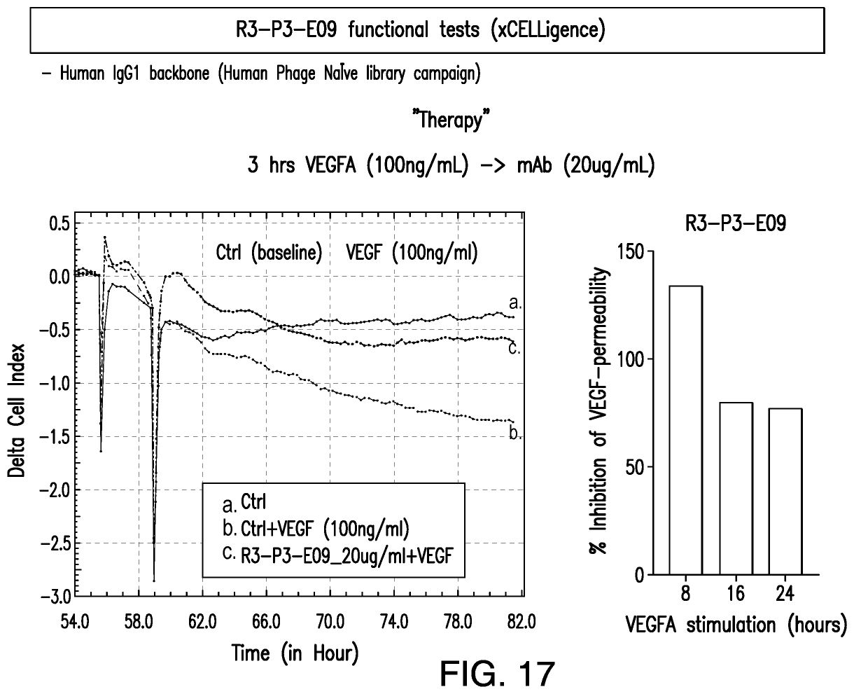

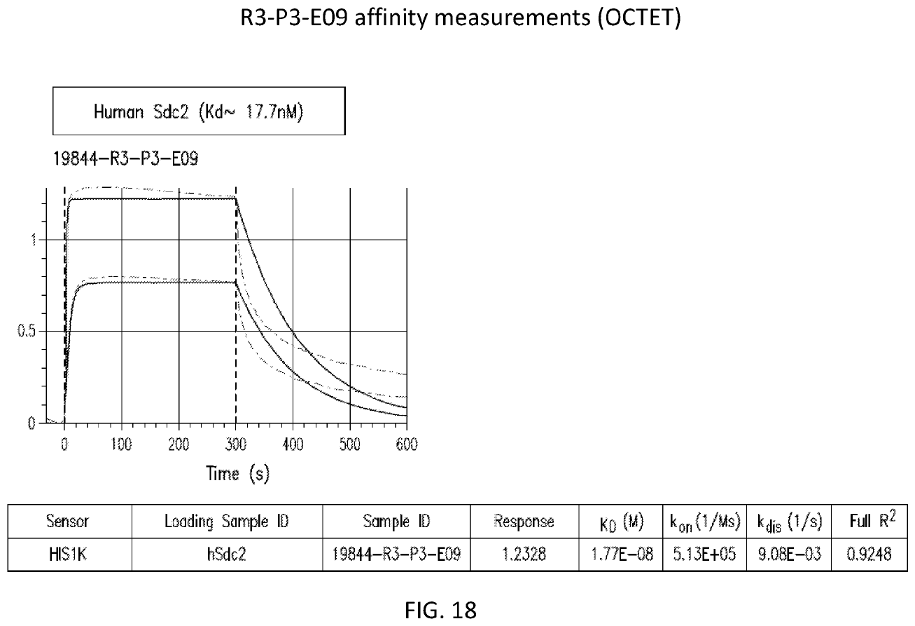

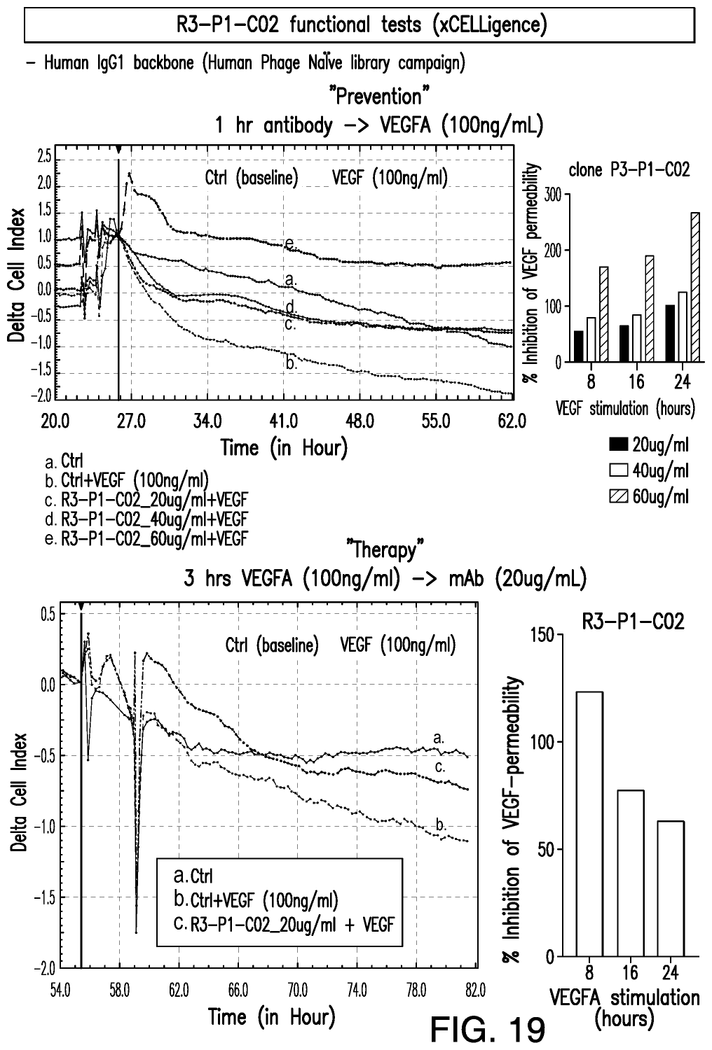

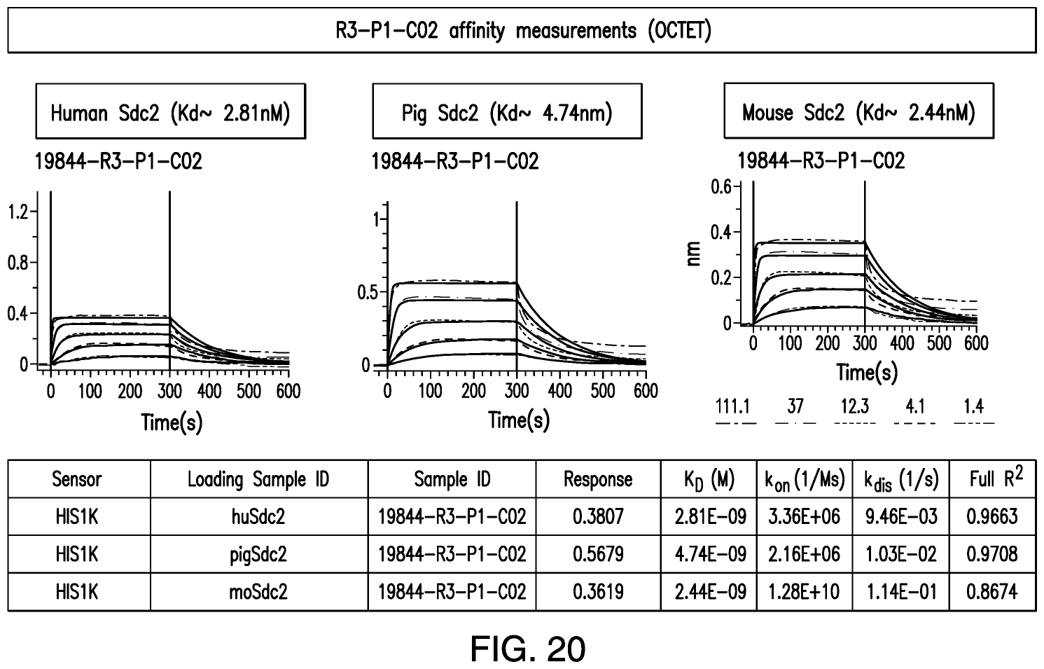

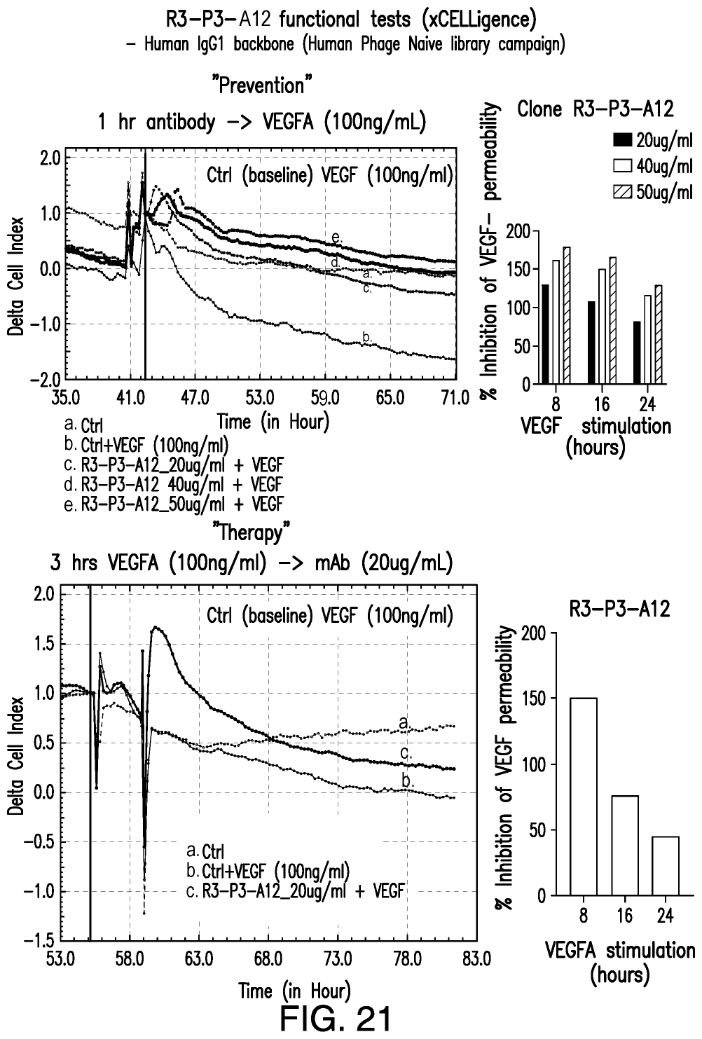

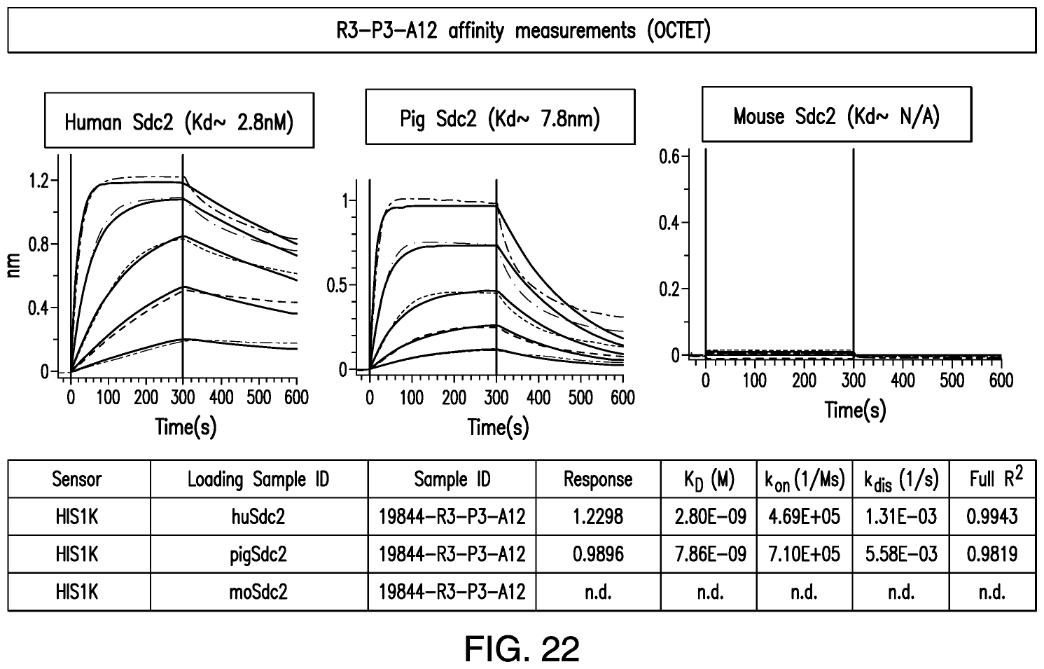

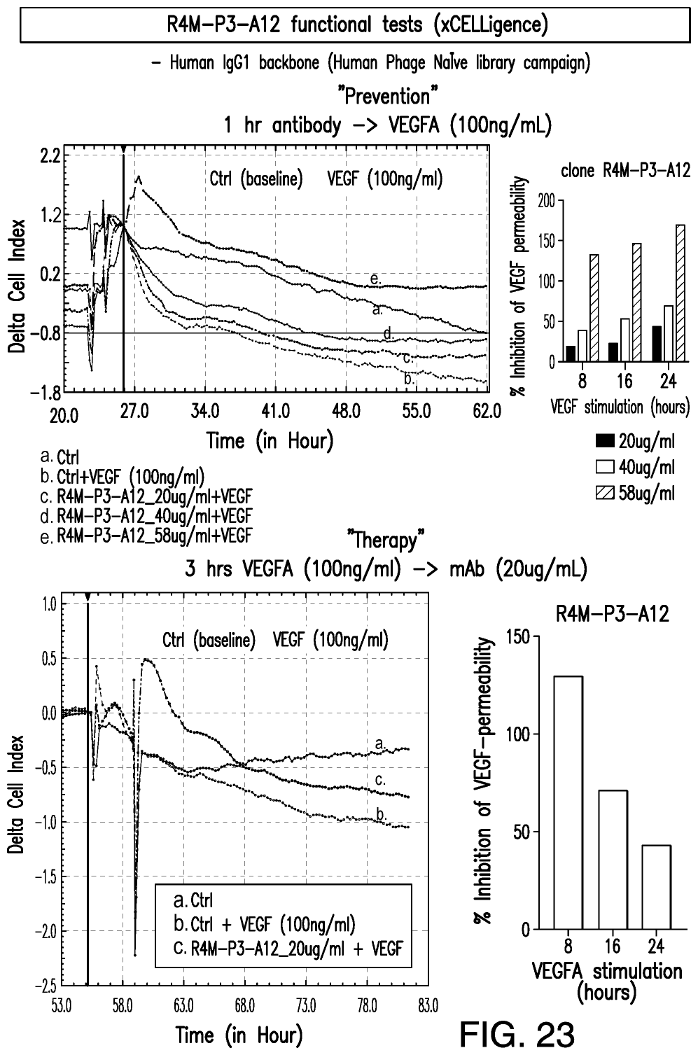

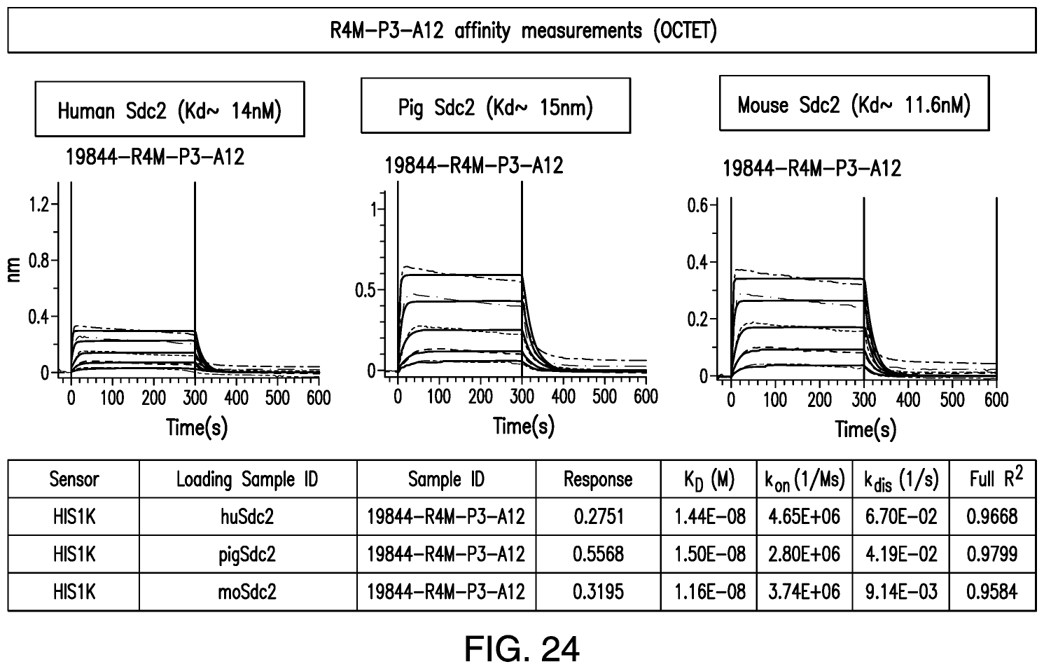

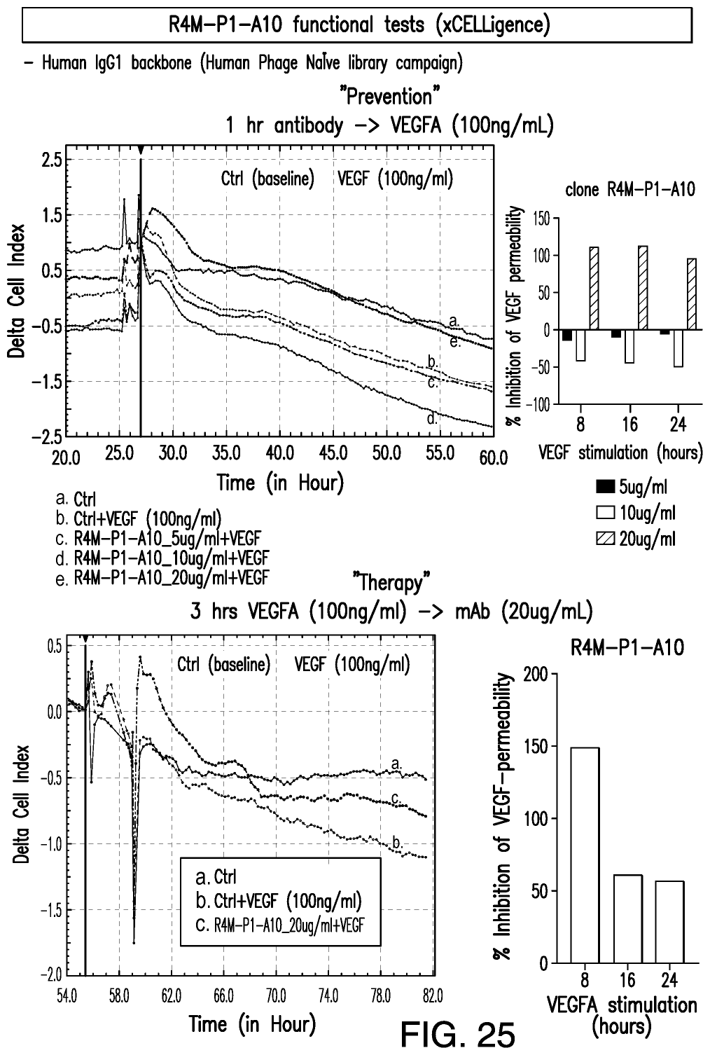

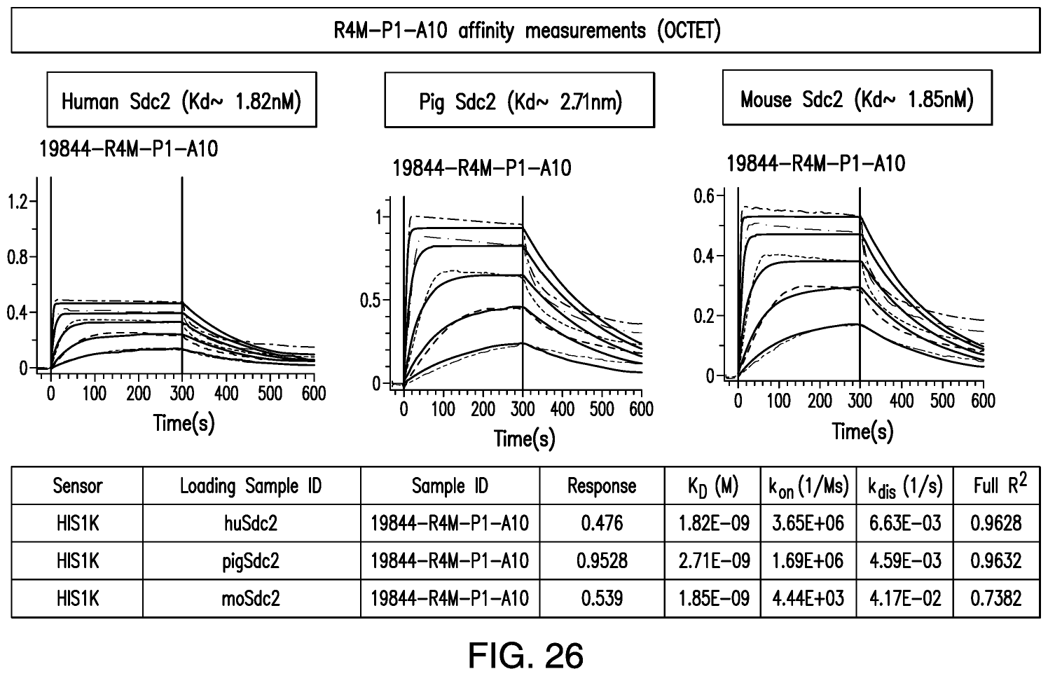

In order to characterize the binding affinity and in vivo/in vitro functional ability of the twelve clones generated by the previous studies and disclosed in Table 1, a series of assays were performed on each clone. These assays measured the affinity of each antibody clone for human, pig, and mouse Sdc2 using an Octet® binding assay system. Additionally, the functional ability of each clone to inhibit VEGFA-driven permeability (see Santiago, et al. (2021) J Am Heart Assoc. 10(18):e020521) was assessed in two therapeutic settings: a “prevention” study in which three concentrations of antibody (5 μg/ml, 10 μg/ml, 15 μg/ml, 20 μg/ml, and/or 40 μg/ml depending on the study) were pre-incubated for an hour before administration of 100 ng/ml of VEGFA, and a “treatment” study in which 3 hours of pre-treatment with 100 ng/ml VEGFA were followed by treatment with antibody at 20 μg/ml or 40 μg/ml. Results are shown for 8-G17 in FIG. 8 and FIG. 9, 20-H19-AB in FIG. 10 and FIG. 11 (FIG. 12 for monkey ECs), R3-P3-C11 in FIG. 13 and FIG. 14, R4M-P3-E06 in FIG. 15 and FIG. 16, R3-P3-E09 in FIG. 17 and FIG. 18, R3-P1-C02 in FIG. 19 and FIG. 20, R3-P3-A12 in FIG. 21 and FIG. 22, R4M-P3-A12 in FIG. 23 and FIG. 24, and R4M-P1-A10 in FIG. 25 and FIG. 26. In summary, and without wishing to be bound by theory, in these particular in vitro studies, clones 8-G17 and H19 were able to reduce VEGFA-induced endothelial permeability by 95% and 65%, respectively.

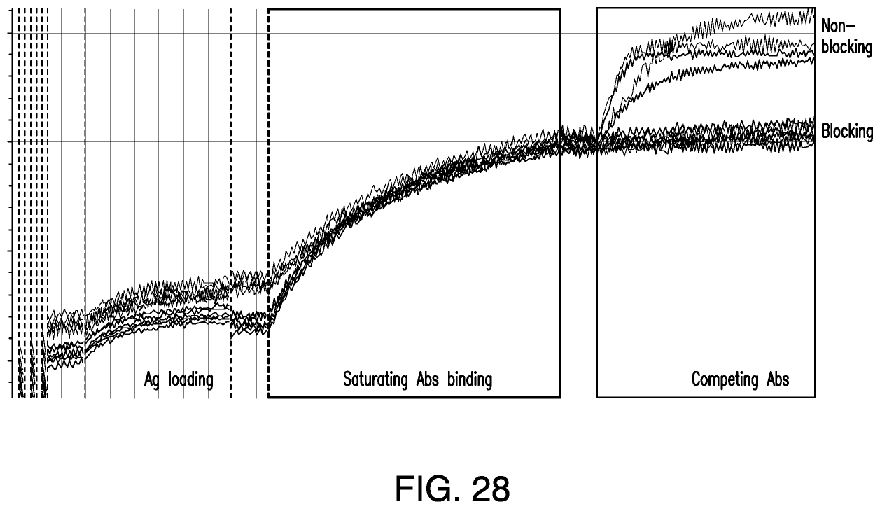

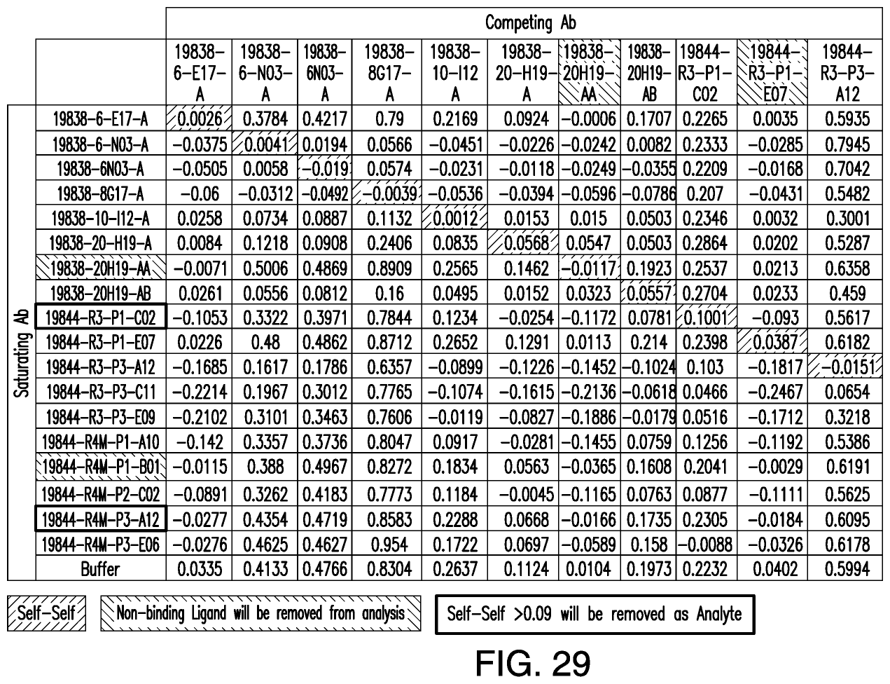

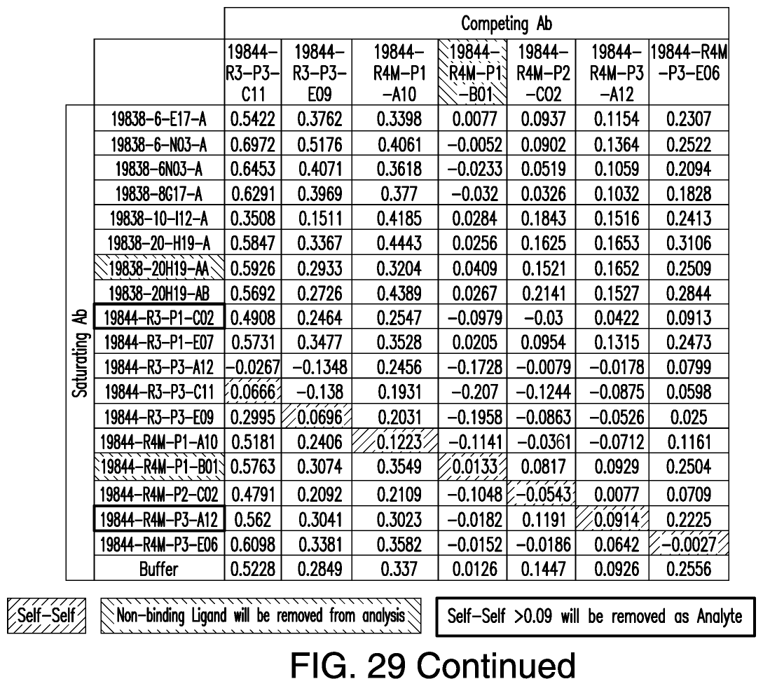

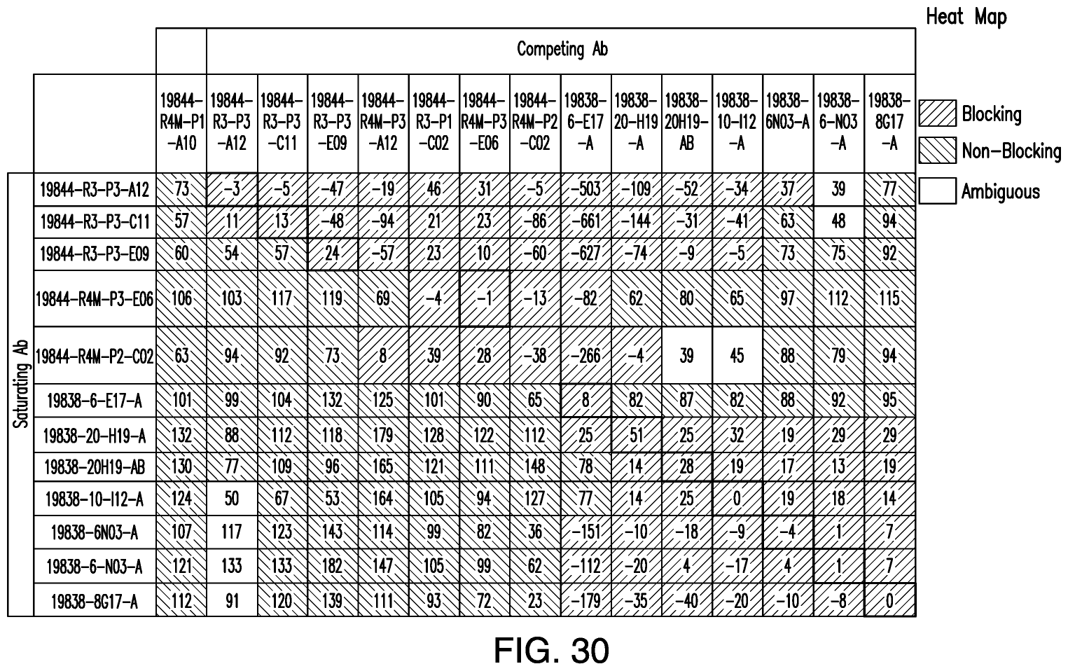

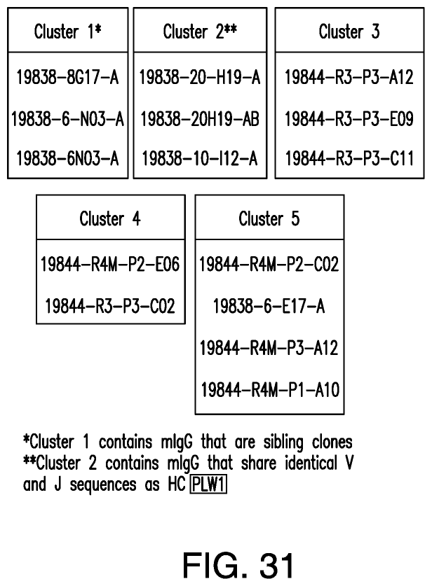

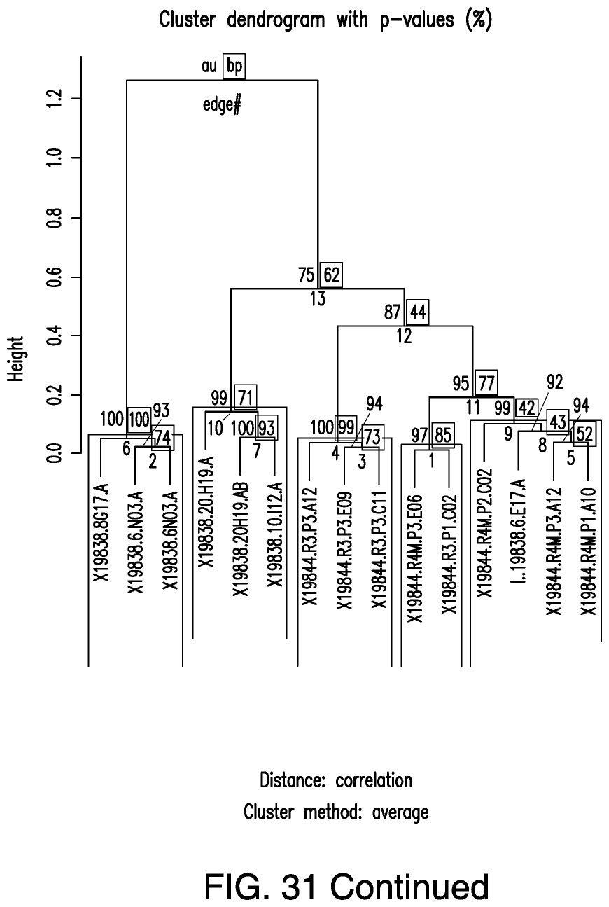

The Octet® platform was also used to perform epitope binning studies using the 18 clones listed in FIG. 27. These studies were performed in a tandem binning assay format with human Sdc2, used as the antigen (concentration of 100 nM). Antigen loaded biosensors were dipped in a saturating solution of antibody (25 μg/ml) for 240 seconds followed by a competing antibody at 5 μg/ml for 180 seconds. An example of the raw sensorgram is shown in FIG. 28. Here, the saturating antibodies were 19838-10-112-A and 1983-20-H19-A. A matrix of raw blocking data is illustrated in FIG. 29. Clones that did not show binding to the antigen in the saturation step are highlighted in grey (19838 20H19 AA, 19844 R3 P1 E07, 19844 R4M P1 B01). Since these clones did not bind to antigen significantly in this assay, they were removed in both orientations. Clones with extremely fast off rates (19844 R3 P1 C02, 19844 R4M P1 A10, 19844 R4M P3 A12) are highlighted in blue. Since saturation is not possible to achieve saturation with these clones, they are removed in as ligands, but retained in the analyte orientation in the analysis. A table summarizing the results of epitope binning of the 18 antibodies against huSdc2 is illustrated in FIG. 30. Data is normalized, where the raw binding signal from Competing Ab for a given Saturating Ab is divided by binding in the absence a Saturating Ag (“buffer”). The threshold determining the matrix color is adjustable and dependent upon Ag-Ab binding. The majority of the blocking interactions have normalized values of less than 51, but sensorgrams for individual interactions were individually examined and may have been recolored to “Ambiguous” if the sensorgram did not appear to match the blocking or non-blocking assignment based on the normalized value threshold. Table rows are based on similar looking binding profiles. Note that the coloration/cutoff is subjective, yellow color added to represent injections with ambiguous or very weak sandwiching signals. FIG. 31 illustrates a clustergram of the epitope binning studies, which was generated from the normalized binning data by importation into the statistical computing program R (The R Project for Statistical Computing: at r-project.org) and generation of a clustergram with the pvclust package.

The y-axis of the clustergram, referred to as Height, is a measure of dissimilarity between antibodies. AU (Approximate Unbiased) p-values were computed using multi-scale bootstrap resampling and confirmed bin assignments (shown in unboxed numerical values). As result, five distinct clusters emerged at an AU p-value cut-off at 95 (shown as the five boxed groups). Each AB cluster share similar but not necessarily identical competition profiles.

### 7.4 Example 4: In Vitro Characterization of 20H19 Antibody

Vascular endothelial (VE)-cadherin is a strictly endothelial specific adhesion molecule located at junctions between endothelial cells. Regulation of its activity or presence at cell contacts is an important step that controls the permeability of the blood vessel wall for cells and substances. For a review, see e.g., Simons et al., Nature Reviews Molecular Cell Biology, 17:611-625 (2016); and Lange et al. Nature Reviews Neurology, 12:439-454 (2016).

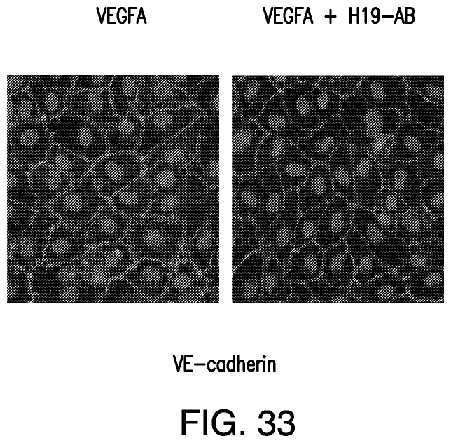

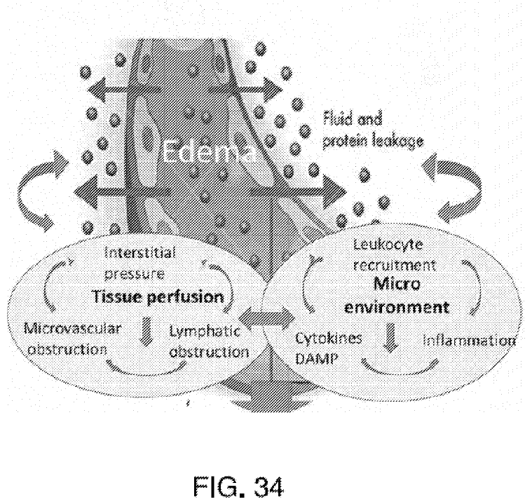

To further characterize the activity of the anti-Sdc2 antibody (20-H19-AB) in inhibiting VEGFA-induced vascular leakage, imaging studies were performed visualizing VE-cadherin as a determinant of endothelial cell contact integrity. FIG. 33 shows the VE-cadherin staining pattern in cell cultures treated with VEGFA in the absence (left panel) or presence (right panel) of the anti-Sdc2 antibody. The nucleus was visualized with DAPI staining and the VE-cadherin complex located on the membranes and cell-cell contacts were visualized with anti-VE Cadherin staining. As shown, integrity of the cell-cell contacts was compromised in cells treated with VEGFA alone, which was restored by treatment with the anti-Sdc2 antibody. This study demonstrated the ability of the anti-Sdc2 antibody in preserving endothelial monolayer integrity when cells are stimulated with VEGFA.

### 7.5 Example 5: In Vivo Characterization of 20H19 Antibody in Non-Human Primates

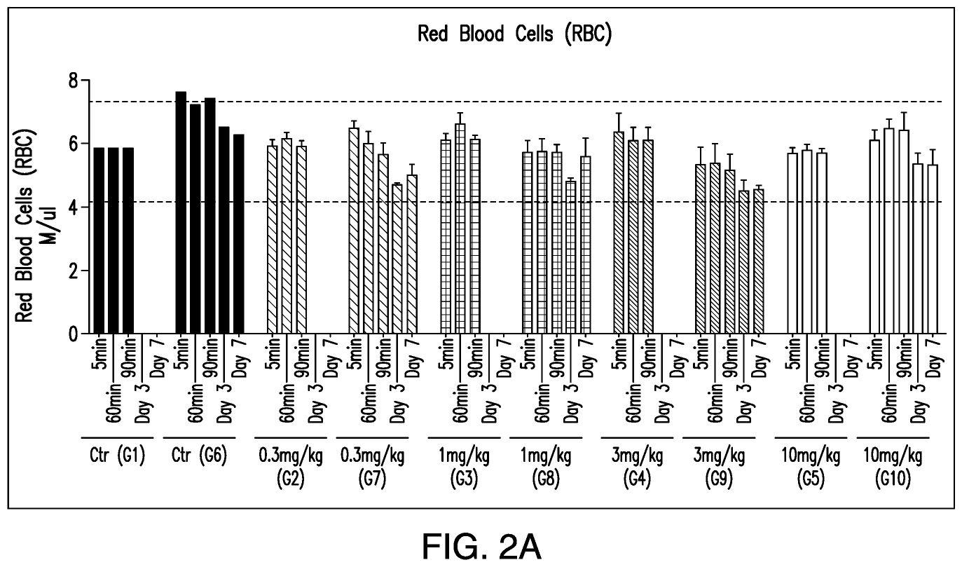

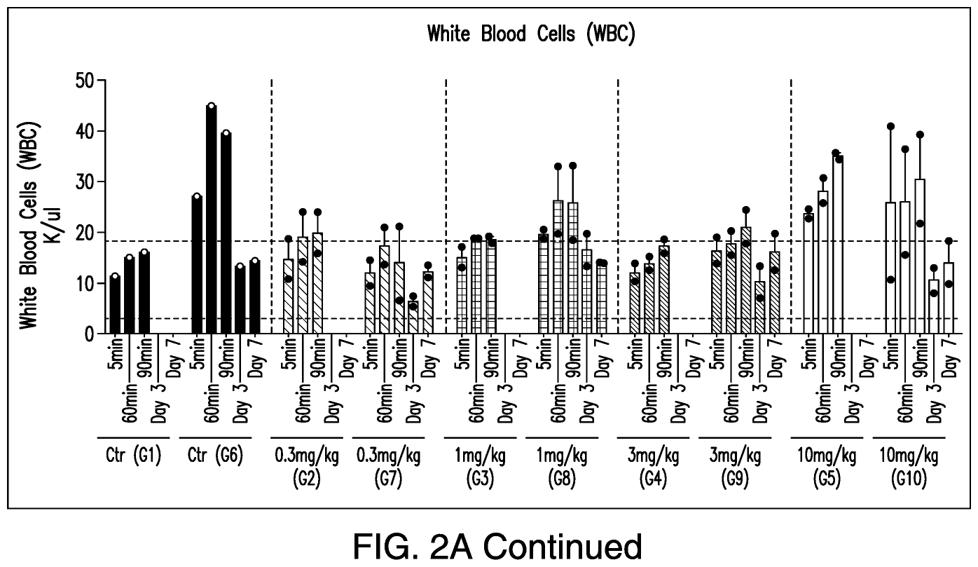

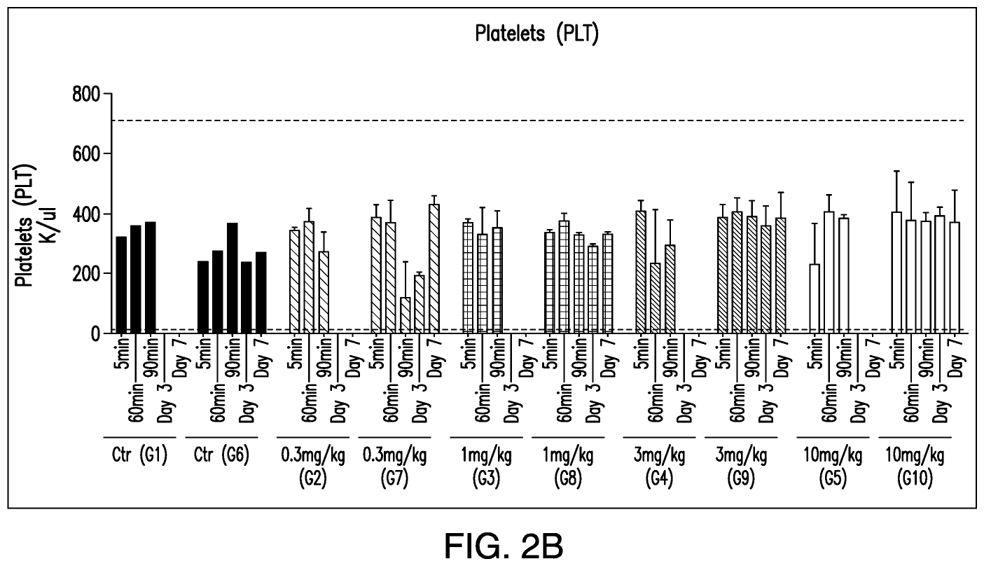

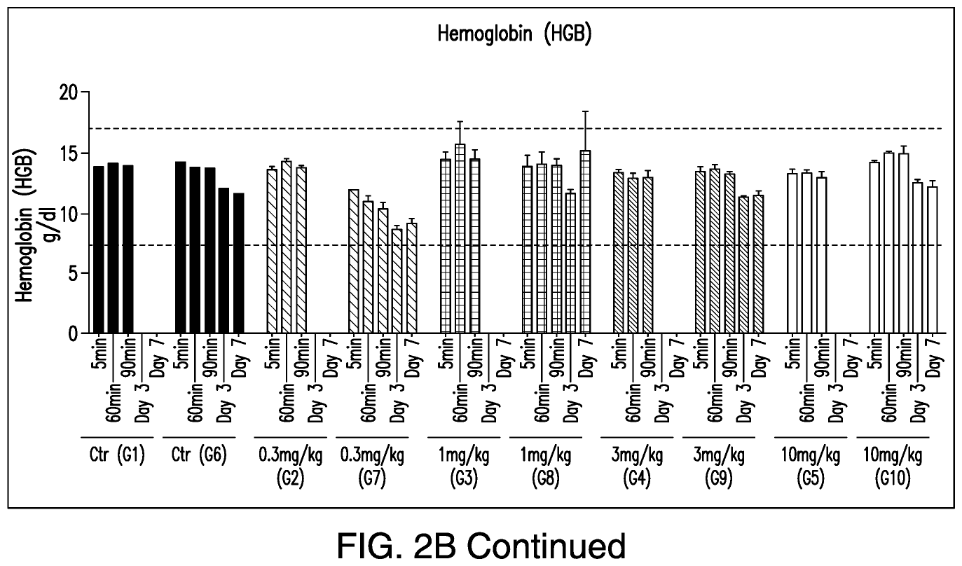

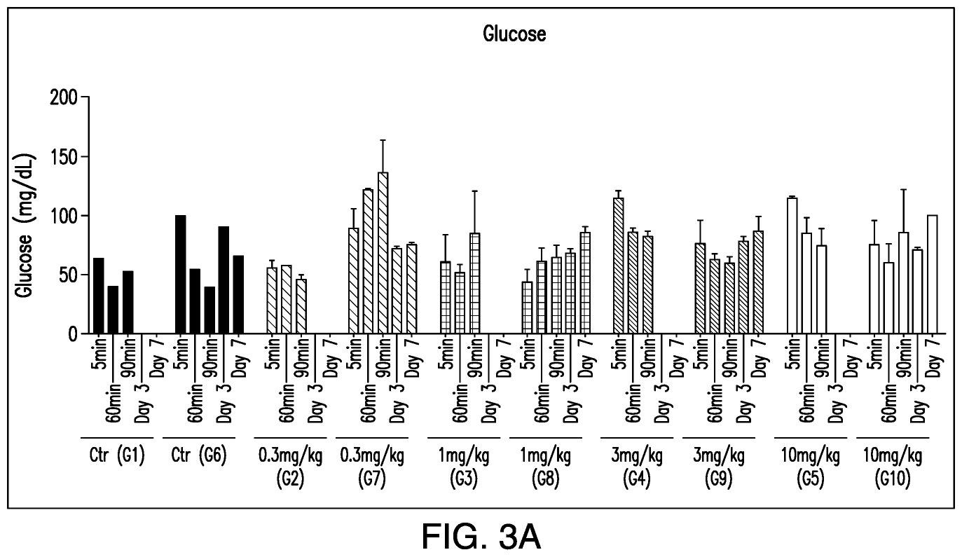

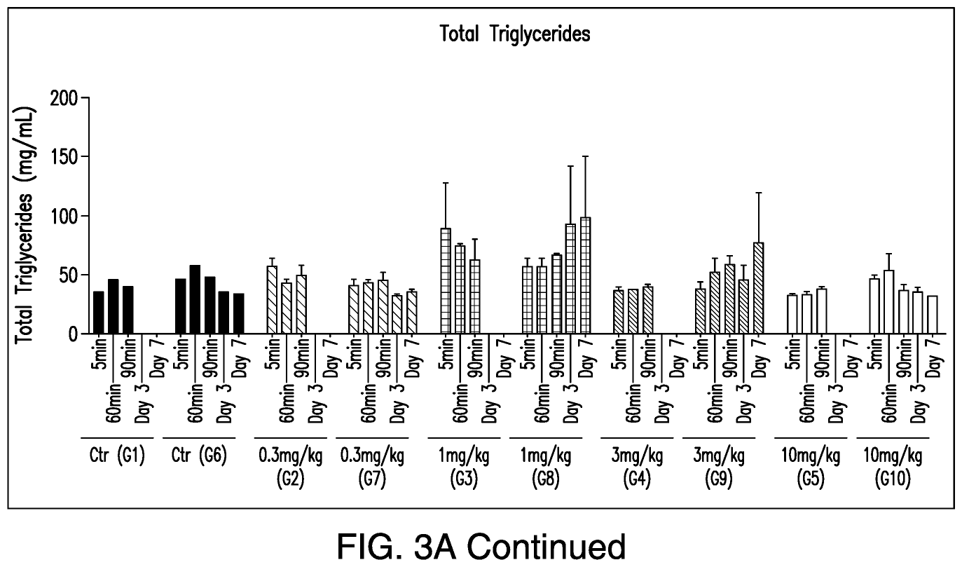

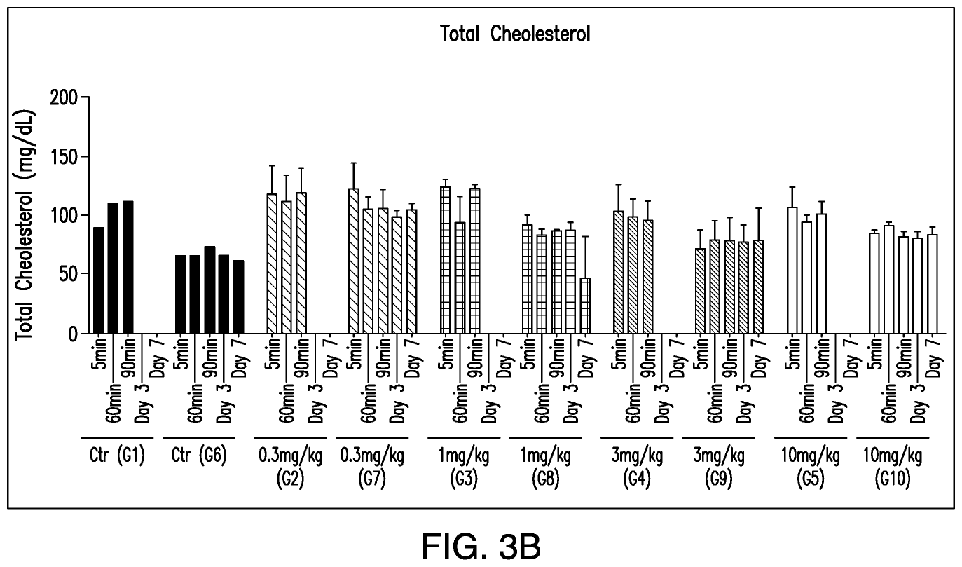

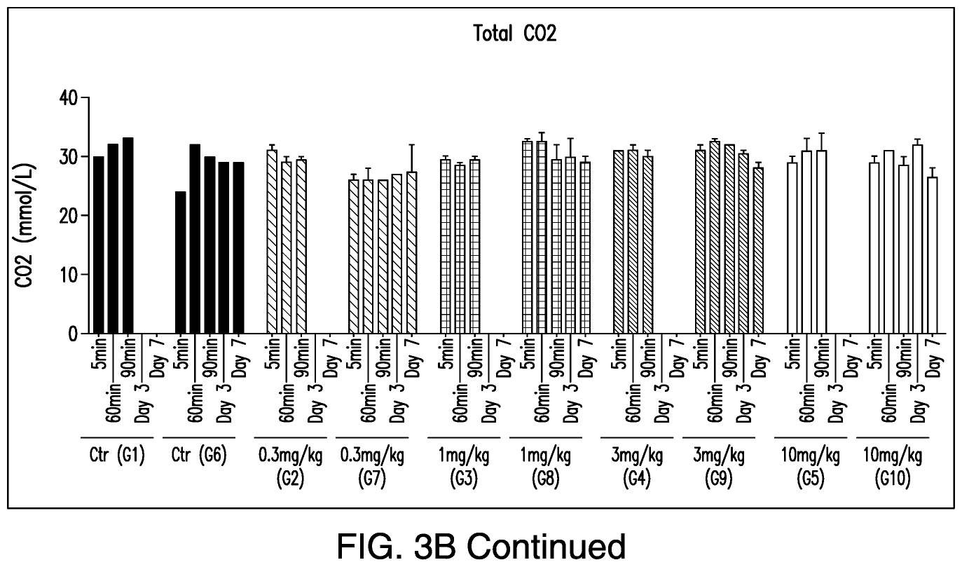

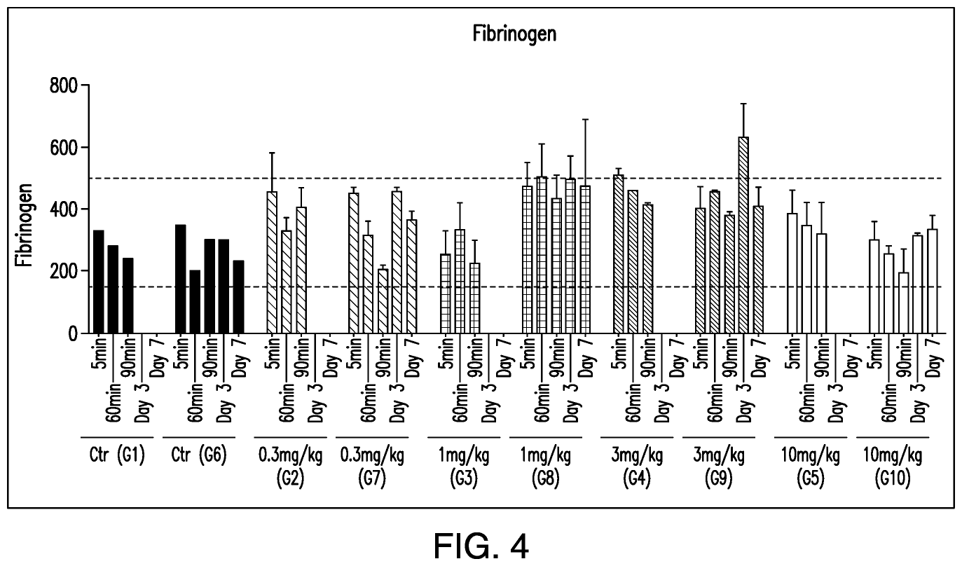

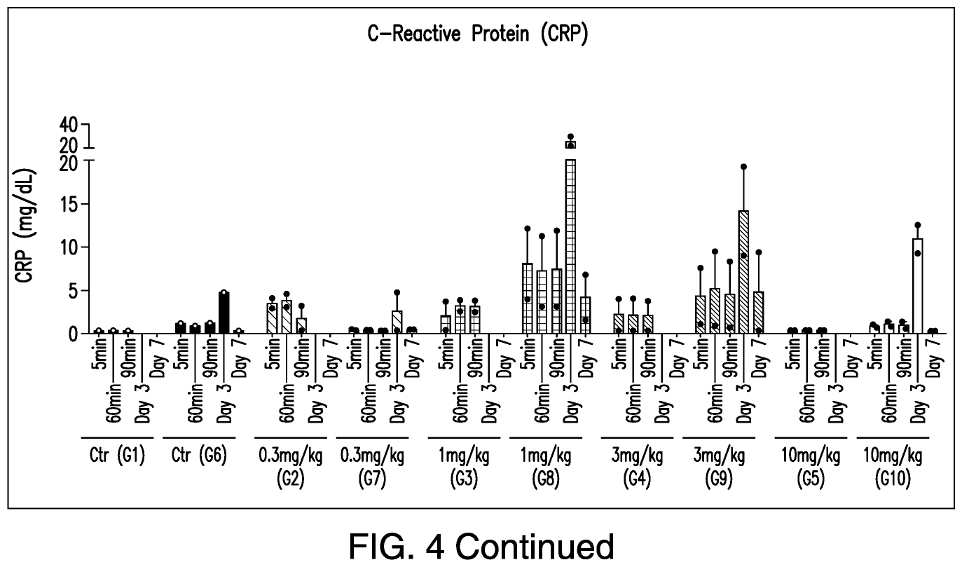

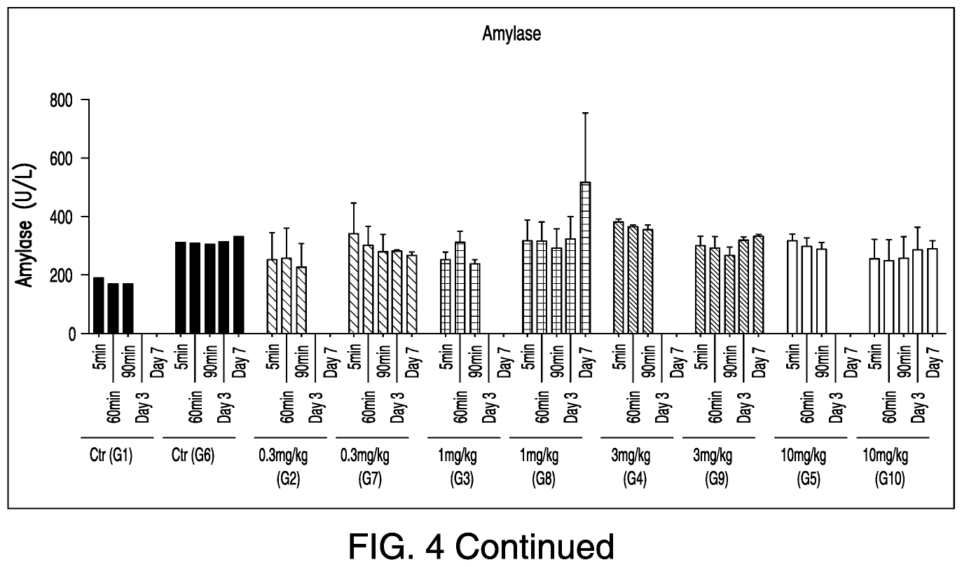

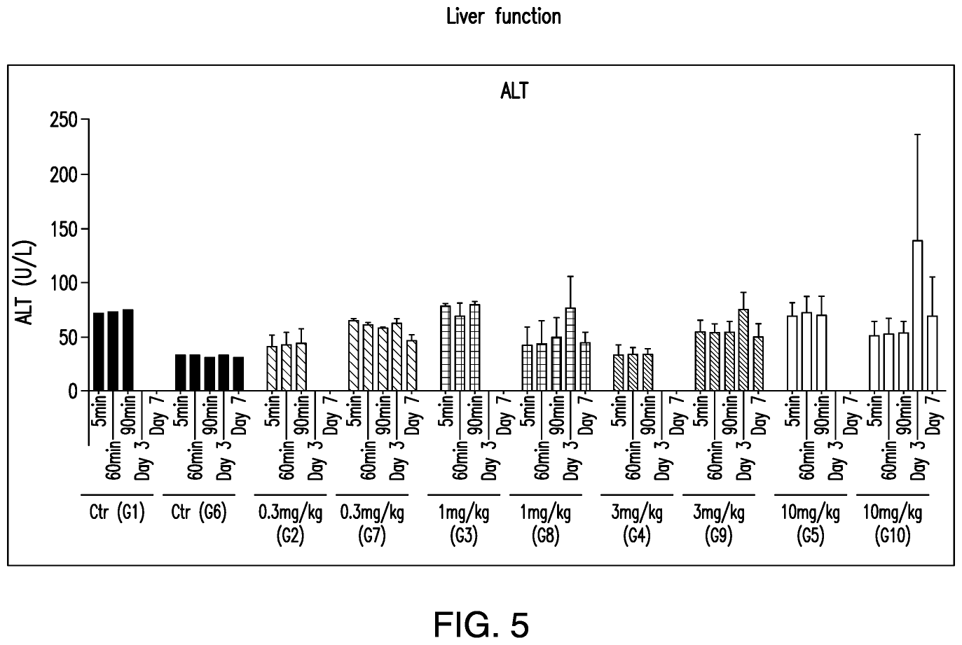

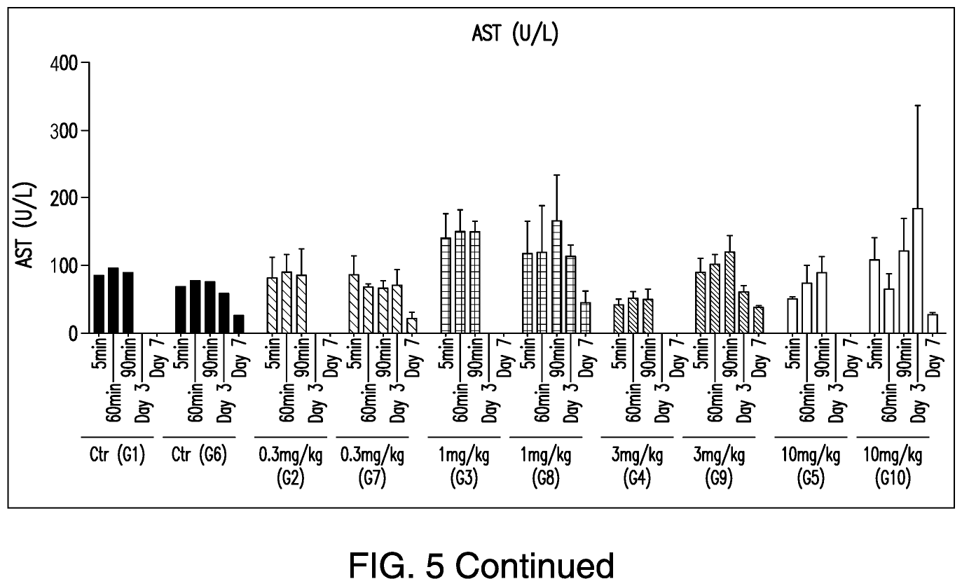

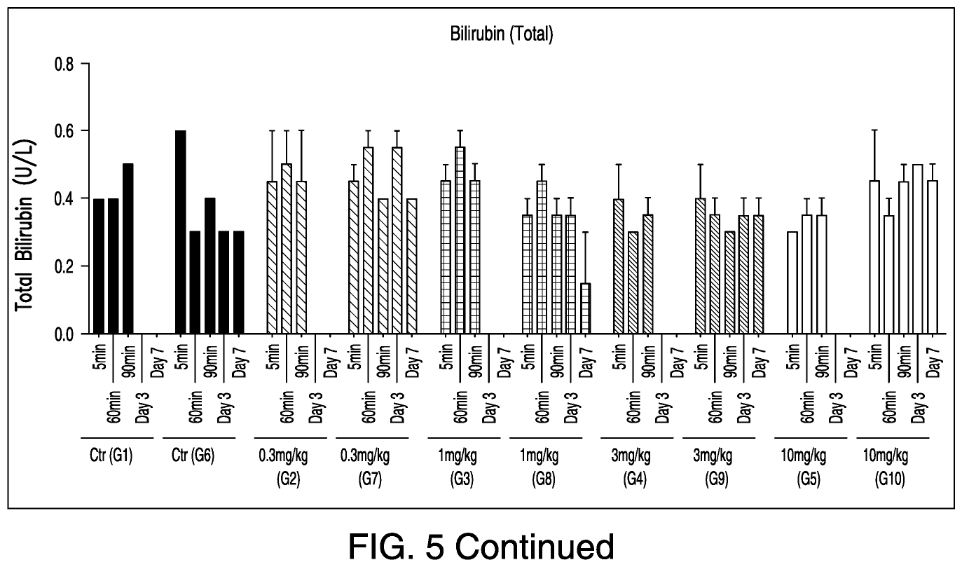

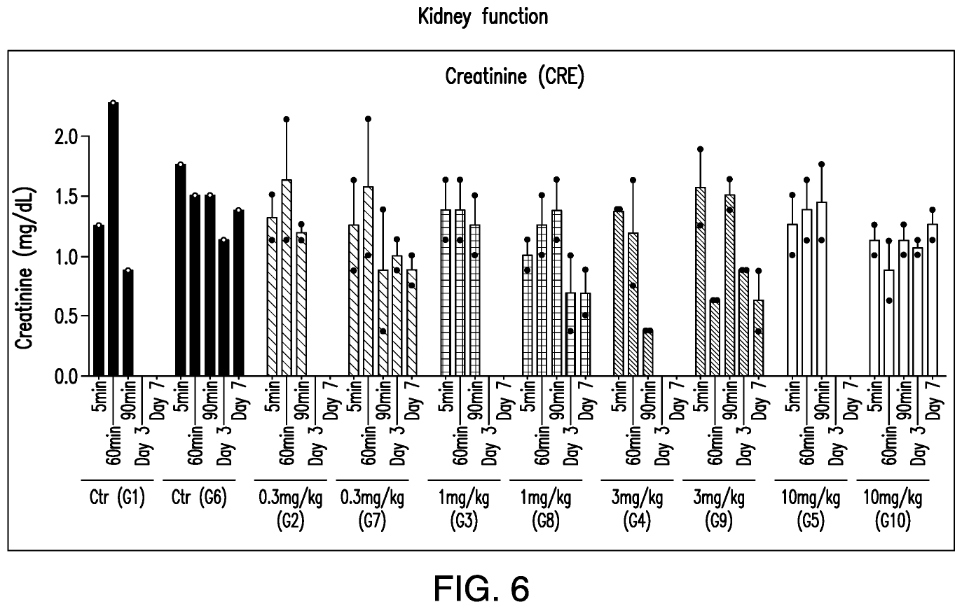

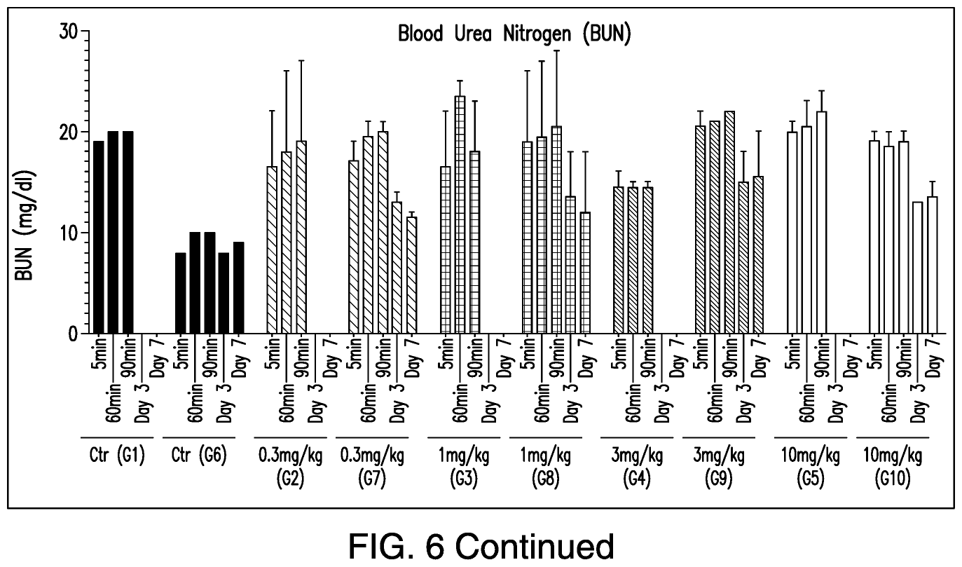

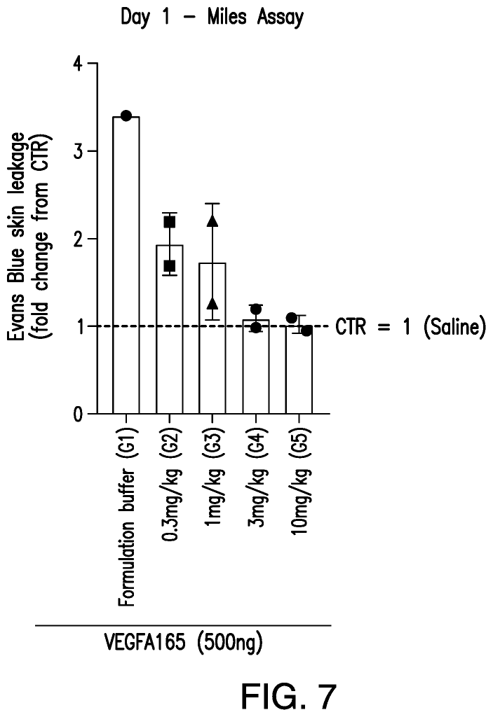

As further development of the anti-Sdc2 antibodies of the current disclosure, a series of single-dose safety and biokinetics studies were undertaken in which the 20-H19-AB antibody was administered to cynomolgus monkeys (Macaca fascicularis) at dosages ranging from 0.3 mg/kg, 1.0 mg/kg, 3.0 mg/kg, and 10.0 mg/kg. Animals were monitored over the course of seven days for several biomedical parameters including hematology and coagulation, clinical chemistry, vital signs and hemodynamic markers, body and organ weights, and any abnormal gross or macroscopic findings. Animals were then sacrificed, and histopathology of the heart, brain, kidney, liver, lung, gonad, and spleen was performed. Additionally, a Miles assay for vascular permeability was performed. FIG. 2A and FIG. 2B show changes in red and white blood cell counts and platelet and hemoglobin levels at various timepoints for each treatment group. FIGS. 3 and 4 show clinical chemistry parameters at various timepoints for the treatment groups, including glucose and triglycerides (FIG. 3A), total cholesterol and CO₂ (FIG. 3B), fibrinogen, CRP, and amylase (FIG. 4). Liver (FIG. 5) and kidney (FIG. 6) function was also assessed. Thus far, these data demonstrated that antibody administration was well tolerated with minimal toxicity, as suggested by the (lack of) behavioral changes and unchanged hematological and biochemical blood parameters. Lastly, a Miles assay was performed at 24 hrs. following mAb injection, to observe the effect on vascular leakage in treated animals. As shown in FIG. 7, vascular leakage decreased with increased amounts of antibody, with 3 mg/kg and 10 mg/kg being statistically identical to a no-dye vehicle control (dotted line). Without wishing to be bound by theory, these data suggested that anti-Sdc2 antibodies retain their functional capability when administered in vivo and can be used to inhibit or otherwise regulate vascular permeability in a clinical setting.

### 7.6 Example 6: Evidence of Safety of Anti-Sdc2 Immunotherapy

Both Sdc2 knockout mice and Y951F transgenic mice which produce an anti-Sdc2 antibody mimetic were observed/reported to develop normally, healthy, and have a long-life span that is comparable to wild type mice. Furthermore, systemic injection of anti-Sdc2 antibody in mice did not cause observable side effects in both short and long terms. See e.g., Claesson-Welsh et al. Trends in Molecular Medicine, 27:314-331 (2021) (ePub 2020), and Corti et al., Nat Cardiovasc Res., 1:518-528 (2022).

Additionally, systemic administration of anti-human Sdc2 antibody at a dosage of up to 10 mg/kg intravenous bolus was well-tolerated and safe in non-human primate model animals, with no morality, no behavioral changes, no apparent biochemical or hematological changes (CBC, liver and kidney function, lipid panel, inflammation markers), and no histological changes observed in brain, liver, kidney, lung, heart and spleen observed in both short and long terms (see e.g., Examples 5 and 7 herein).

These data demonstrate anti-Sdc2 immunotherapy to be safe and well-tolerated for treating Sdc2 associated conditions or disease in a subject in need thereof.

### 7.7 Example 7: Efficacy of 20H19 Antibody in Treating Ischemic Stroke

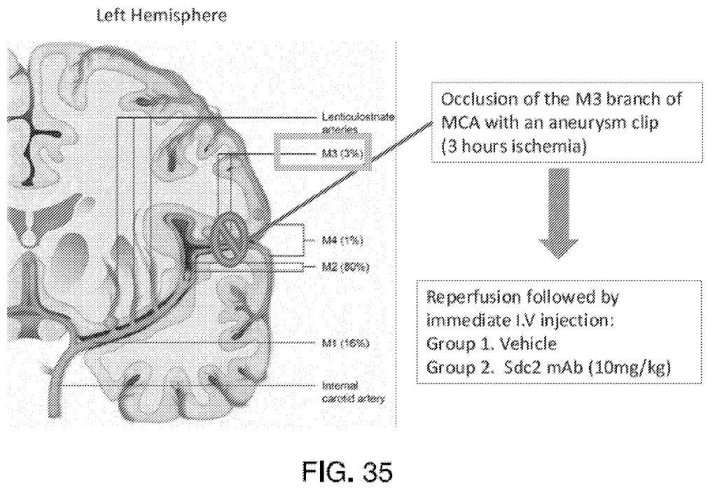

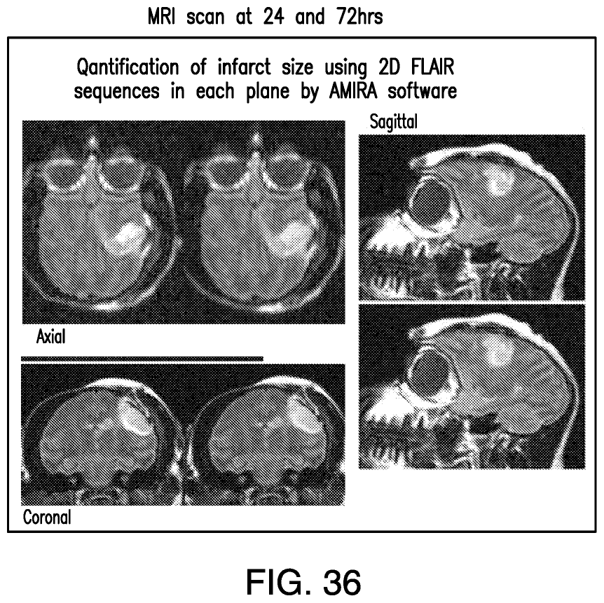

Previously, it has been shown that rabbit anti-Sdc2 polyclonal antibodies was effective in ameliorating brain lesions in a stroke mouse model. See, e.g., Corti et al., Nat Cardiovasc Res., 1:518-528 (2022). To further evaluate the use of anti-Sdc2 antibody therapy for treating ischemic stroke, a non-human primate ischemic stroke model was created using monkeys. Particularly, as shown in FIG. 35, a craniotomy over the left temporal bone was performed on adult male monkeys to expose the brain surface, and the M3 segment was located approximately 5 mm within the Sylvian fissure. The M3 branch of the middle cerebral artery (MCA) was surgically isolated and occluded with an aneurysm clip for 3 hours. At the end of the occlusion, the clip was removed, and reperfusion was visually confirmed. Brasiliense et al., Contemporary Neurosurgery 39(2):1-7 (2017). MRI scans were performed 24 hours and 72 hours following the surgery to confirm and quantify the infarct size using 2D FLAIR sequences by AMIRA software (FIG. 36)

An intravenous infusion of either test article, which was an IgG4 version of the humanized anti-hSdc2 antibody (20-H19-AB-cyIgG4, 10 mg/kg) or vehicle began immediately after the onset of reperfusion.

To control for effects of the surgical procedures, a sham surgery was performed where the M3 branch was isolated for 3 hours but not clamped. The sham animals did not receive an infusion. Additionally, two untreated controls were included in the vehicle group. The untreated controls had the same surgery without any infusion after occlusion.

MRI sessions were performed at 1 and 3 days following the MCA occlusion procedure. As shown in FIG. 38, regions of interest (ROI) were constructed using 2D FLAIR sequences in each axis (Axial, Coronal, Sagittal) to score hyperintensities associated with the infarct. A single analyst blinded to treatment rated the 2D FLAIR images with QC and data analysis were performed using the ANOVA test followed by the NP Holm-Sidak tests performed by the Study Director.

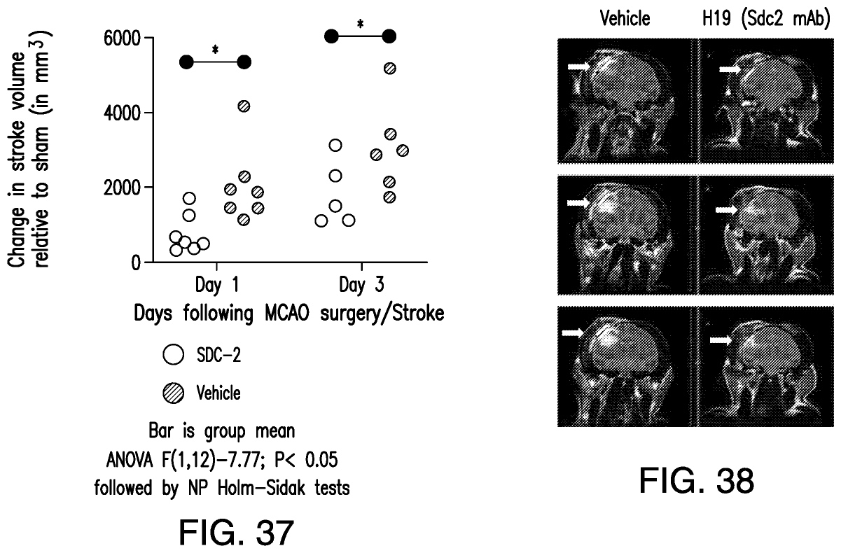

FIG. 37 shows quantitation of the change in the stoke volume in animals received the anti-Sdc2 antibody treatment or vehicle 1 day or 3 days after the MCA occlusion procedure. The change in the stoke volume was measured relative to animals receiving the sham surgery. Each group contained at least five duplicated experiments. FIG. 38 shows exemplary reconstructed images of ROI of a monkey received IgG vehicle (left) in comparison to another monkey received the anti-Sdc2 antibody treatment (right) 1 day after the MCA occlusion procedure. As shown, a single i.v. injection led to 50-60% stroke size reduction and edema formation at day 1 and day 3 post-MCA occlusion, indicating the therapeutic window was longer than 24 hours. These results demonstrate in vivo efficacy of anti-Sdc2 therapy in treating ischemic stroke.

During these studies, no safety issues or adverse side-effect were observed in extensive use in the non-human primate model.

### 7.8 Example 8: Anti-Sdc2 Antibody can Effectively Reduce Vascular Leakage in Mice

A mouse model of wet AMD was generated by laser-induced choroidal neo-vascularization (CNV). Particularly, mice treated with laser photocoagulation would replicate the main features of wet AMD pathology in humans, demonstrating pathogenic phenotypes including high VEGF expression, inflammation, neovascularization, and responsive to anti-VEGFA therapy. For a review, see e.g., Liu et al. FASEB J. 2017 November; 31(11):4665-4681. A schematic illustration of the experimental paradigm is shown in FIG. 33.

In this study, photocoagulation was performed on Day 0 where a laser beam was used to induce lesion on the animal's retinal pigment epithelium (RPE). Then a rabbit anti-mouse Sdc2 polyclonal antibody (Ab3) that binds to the Dep-1 binding region in Sdc2 was injected either intravitreally or systemically (through tail vein) to the animals on Day 3 and Day 6 after the photocoagulation procedure. In a positive control group, an anti-VEGF antibody was administered instead of the anti-Sdc2 antibody. In a negative control group, empty vehicles were administered. On Day 7, fundus fluorescein angiography (FFA), optical coherence tomography (OCT) and electroretinogram (EGR) were performed to check the treatment effects of anti-Sdc2 antibody for AMD.

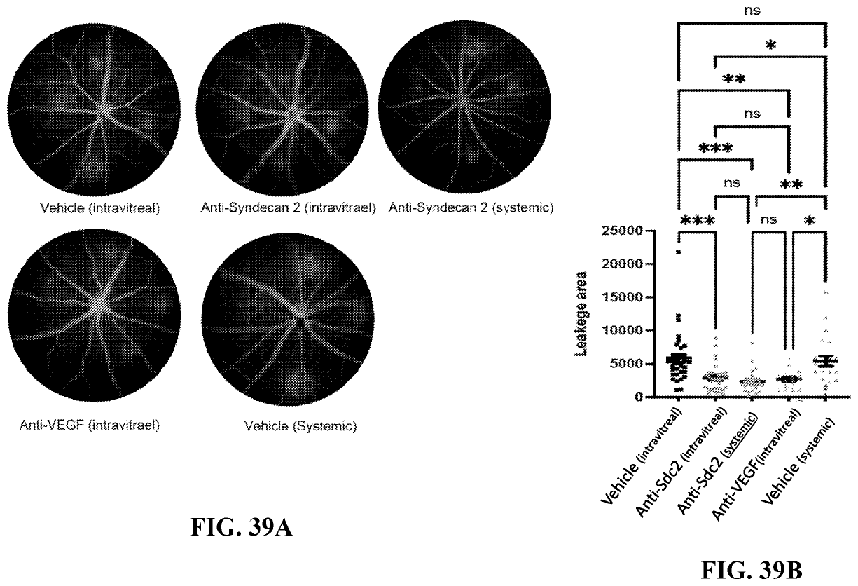

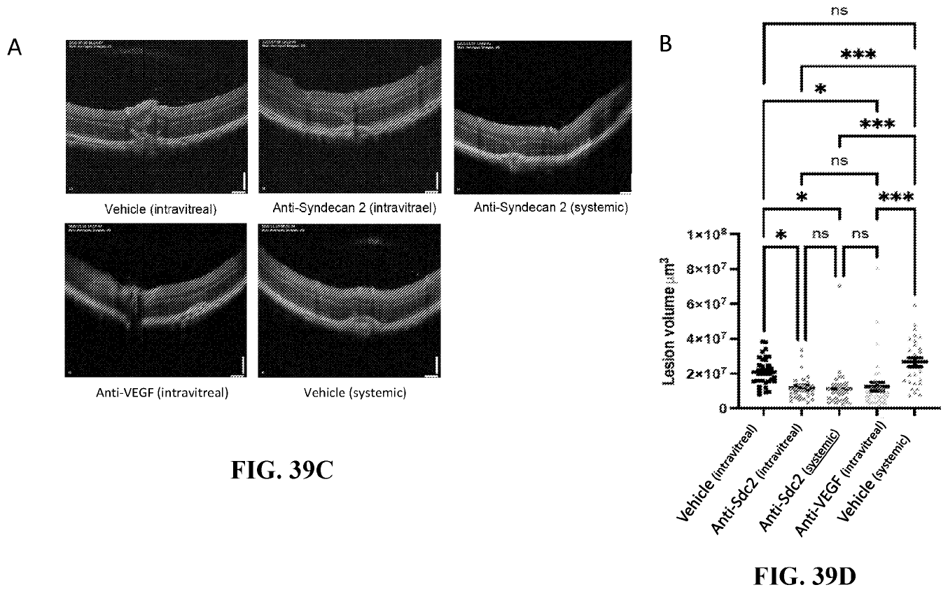

FIG. 39A shows images of FFA from mice in the different groups on Day 7 after laser burns, showing formation of lesions (dots) beneath the retinal vasculature (lines), and the lesion areas were quantified in FIG. 39B. As shown, hyper-fluorescent areas which correlate with lesion leakage can be readily detected in the vehicle groups. As shown, the sizes of leakage area in mice treated with anti-Sdc2 antibody were significantly smaller than those treated with vehicle, and the reduction in lesion size in anti-Sdc2 antibody treatment group was comparable to the group treated with the clinical standard anti-VEGF antibody. No significant differences between mice treated with anti-Sdc2 antibody delivered by intravitreal injection and systemic injection (about 50% versus its respective route of administration).

FIG. 39C shows images OCT from mice in the different groups on Day 7 after laser burns, showing formation of lesions on the retinal pigment epithelium, and the lesion volumes was quantified in FIG. 39D. As shown, mice treated with the anti-Sdc2 antibody delivered either by intravitreal injection or systemic injection had similar CNV lesion volumes compared to each other, and to mice treated with the anti-VEGF antibody, all of which were significantly smaller (about 45% reduction) than mice treated with vehicle. In both routes of administration, Sdc2 blockage demonstrated efficacy in reducing CNV lesion leakage and volume equivalent to intravitreal anti-VEGF inhibition. These data demonstrate that treatment with the anti-Sdc2 antibody effectively reduced the volume and leakage area of the laser-induced lesion in the AMD model, similar to the treatment using an anti-VEGF antibody. These data demonstrate that anti-Sdc2 therapy is as effective as anti-VEGF treatment which is current gold-standard for AMD therapy. This study also demonstrates the potential to eliminate intravitreal injections (and ensuing side effects) for the treatment of AMD.

### 7.9 Example 9. Anti-Sdc2 Antibody has No Significant Effect on Retina Function

This study used electroretinography (ERG) to examine the effects of anti-Sdc2 antibody on retinal function of the AMD disease model mice. The AMD model mice were generated by photocoagulation as described in Example 7 on Day 0. Then a rabbit anti-mouse Sdc2 polyclonal antibody (Ab3) was delivered intravitreally to the animals on Day 3 and Day 6 after the laser burns. ERG was performed on Day 7 to check the treatment effects.

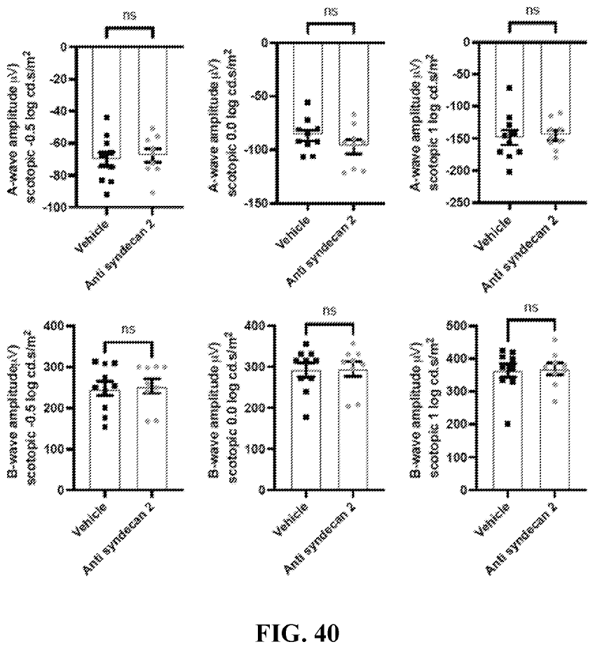

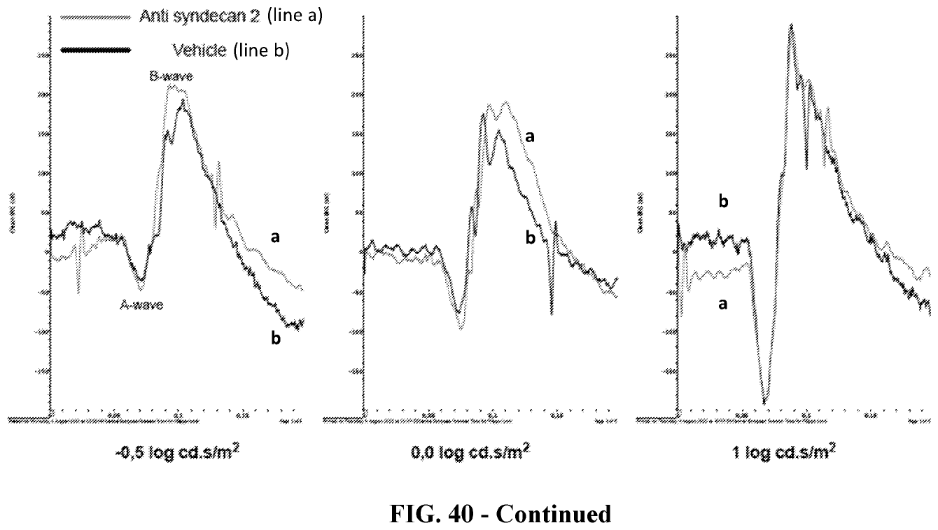

FIG. 40 shows the ERG results in mice treated with anti-Sdc2 antibody or empty vector vehicle. A-wave and B-wave amplitudes at three different light intensities were detected. No significant differences were observed between the mice treated with anti-Sdc2 antibody and mice received the empty vehicle. These data demonstrate that treatment with the anti-Sdc2 antibody has no obvious effects on retinal function of the AMD model mice.

### 7.10 Example 10: Anti-Sdc2 Antibody Inhibited Inflammatory Infiltration

AMD could cause retinal neoangiogenesis and inflammatory infiltration in retinal or choroidal tissues, which would lead to significant visual impairment including loss of visual acuity. To detect the therapeutic effects of anti-Sdc2 antibodies in treating AMD, this study checked the expression of markers for retinal neoangiogenesis and inflammatory infiltration in mice treated with a polyclonal anti-Sdc2 antibody (Ab3), an anti-VEGF antibody, or empty vehicle (control). Immunohistochemistry was used to visualize CD31 and ETS-related gene (ERG). CD31 is known to mainly express by endothelial cells but it is also reported to be expressed in macrophages in the context of inflammation. ERG is known to be a highly specific endothelial nuclear marker and can be used as a neoangiogenesis marker. F4/80 was visualized as a marker for murine macrophages, were used to indicate inflammatory infiltration in mice.

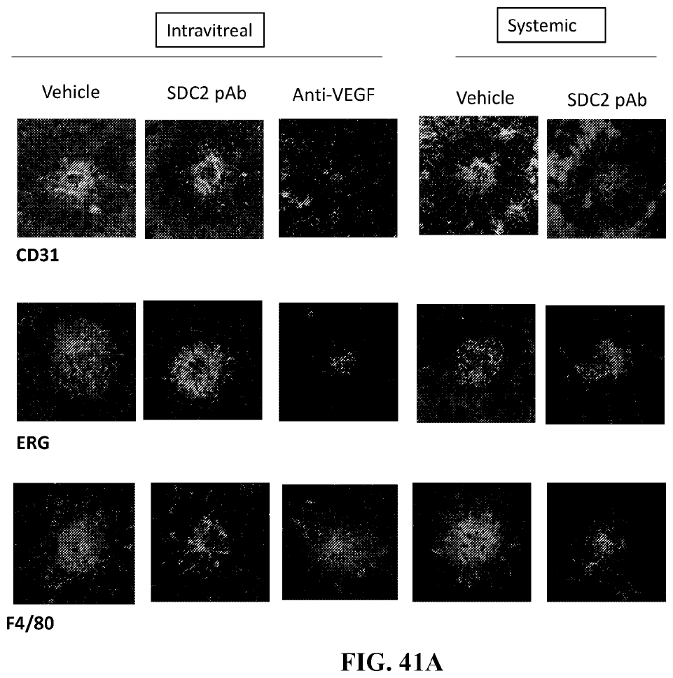

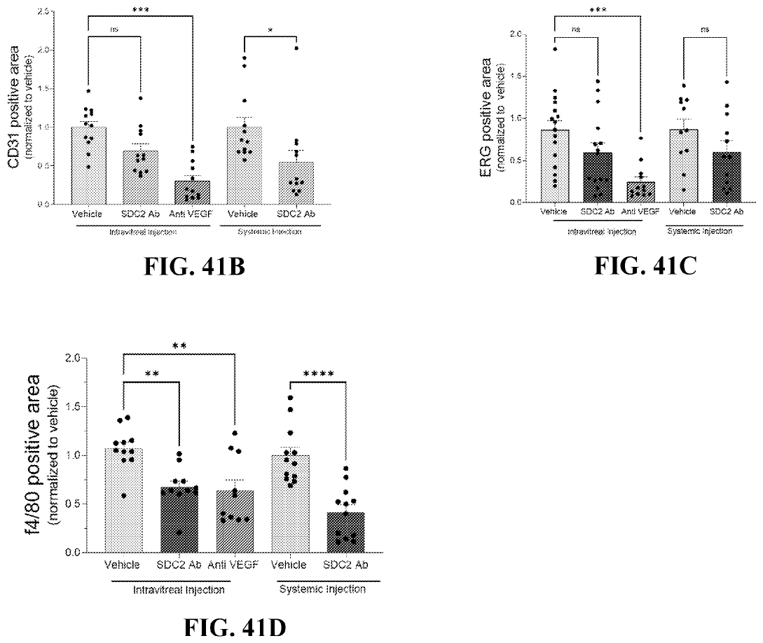

FIG. 41A shows the immunofluorescence staining visualizing CD31, ERG and f4/80, respectively, in choroid/retinal pigment epithelium (RPE) from mice treated with an anti-Sdc2 antibody, an anti-VEGF antibody or vehicle either through intravitreal injection or systemic injection, and the positive areas of those markers were quantified in FIGS. 41B to 41D to present the expression levels of these proteins. As shown in FIG. 41B, in the intravitreal injection group, no significant difference was observed for CD31 expression in mice treated with anti-Sdc2 antibody and mice treated with the vehicle, while CD31 expression was significantly lower in mice treated with the anti-VEGF antibody. In the systemic injection group, CD31 expression was significantly lower in mice treated with the anti-Sdc2 antibody as compared to mice treated with the vehicle. As shown in FIG. 41C, ERG expression was lower in mice treated with the anti-Sdc2 antibody (delivered either by intravitreal injection or systemic injection) than mice treated with empty vehicle, although the differences between the two groups were not statistically significant. In the intravitreal injection group, treatment with anti-VEGF antibody significantly reduced ERG expression as compared to the vehicle or anti-Sdc2 antibody treatment groups.

As shown, intravitreal injection of anti-Sdc2 antibody led to a trend in reduction of CD31 positive area, however, this effect was not significantly different from intravitreal vehicle injection. As expected, intravitreal injection of anti-VEGF antibody led to a strong reduction (70%) of positive CD31 area. Systemic administration of anti-Sdc2 antibody led to a significant reduction (45%) of CD31 positive area compared to control group that received systemic administration of vehicle, but not ERG, a highly specific endothelial nuclear marker. There were no significant differences between ERG positive cells after intravitreal administration of anti-Sdc2 antibody as compared to vehicle. Anti-VEGFA treatment led to strong reduction (75%) in endothelial cell number, confirming its potent anti-angiogenic effect. Overall, these results were in line with previous observation that anti-Sdc2 antibody does not affect angiogenesis but act selectively to block VEGFA-induced permeability.

As shown in FIG. 41D, expression of f4/80 in mice treated with anti-Sdc2 antibody (delivered either by intravitreal injection or systemic injection) were significantly lower than mice treated with the vehicle, and the reduction in f4/80 expression in anti-Sdc2 antibody treatment group was comparable to the anti-VEGF antibody treatment group. These data demonstrate that systemic injected anti-Sdc2 antibody can inhibit inflammatory infiltration in retinal and choroidal tissues. Particularly, similar reduction of F4/80 positive area (35%) in animals that received intravitreal injection of either anti-Sdc2 antibody or anti-VEGF antibody. More interestingly, injection of systemic anti-Sdc2 antibody led to an even more marked decrease of macrophages presence (60%).

### 7.11 Example 11: Anti-Sdc2 Antibody Inhibited VEGF-Induced Permeability in Mouse Endothelial Cells (Ecs)

VEGF is known to be a potent angiogenic and vascular permeability factor in the pathogenesis of retinal diseases, including AMD (see Miller, et al. Ophthalmology 2013 January; 120(1):106-14). In this study, transepithelial/transendothelial electrical resistance (TEER), which was the measurement of electrical resistance across a cellular monolayer, was used as an indicator for the integrity and permeability of the monolayer. Particularly, an endothelial cell (EC) culture was treated with VEGFA, and permeability were monitor for 12 hours. Then, an anti-Sdc2 antibody (Ab3) was added to the cell culture at 10 μg/ml, 20 μg/ml or 40 μg/ml, and impact of the antibody on the permeability was observed. A control group receiving no VEGFA treatment (baseline control) and a control group receiving only VEGFA and no Ab3 (negative control) were included.

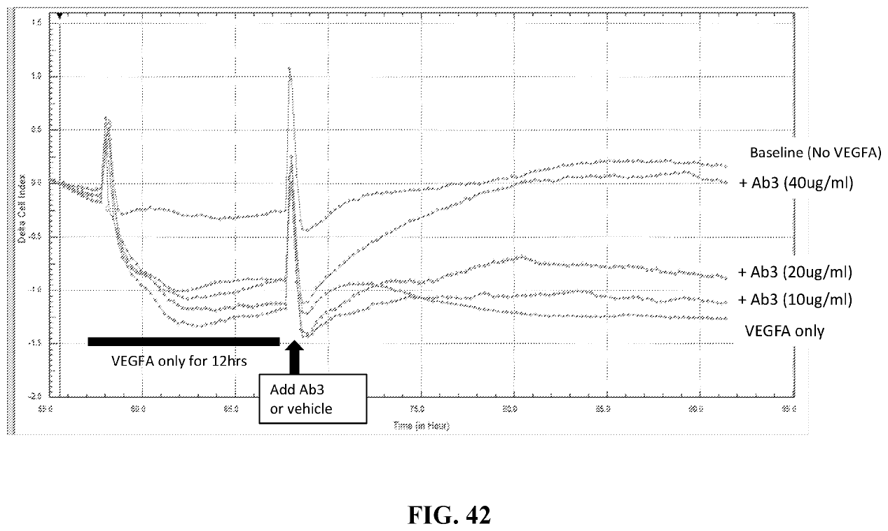

As shown in FIG. 42, anti-Sdc2 Ab3 treatment exhibited a dose-dependent effect on reversing VEGFA-induced permeability in cultured ECs as compared to the negative control group. Permeability of cultured ECs treated with VEGFA plus 40 μg/ml anti-Sdc2 antibody was reverted to a level comparable to the baseline control. These data confirm the effect of anti-Sdc2 antibody in reversing VEGFA-induced endothelial permeability.

### 7.12 Example 12: Mouse Model for Induced Myocardial Infarction (MI)

A mouse model of myocardial infarction (MI) was generated by surgically occluding the left anterior descending coronary artery (LAD) with a suture for a period of time, followed by removing the suture to restore blood flow and tissue reperfusion, sometimes referred to as the ischemia-reperfusion (IR) surgery. To induce AMI with IR surgery in mice, a left-sided thoracotomy was performed on an anesthetized mouse, and a temporary ligation was made to the left ventricle, occluding the left anterior descending coronary artery (LAD). The ligation was temporary mediated by a small piece of tubing. Once the chosen time (1 hour) of ischemia passed, the suture was cut, and tubing removed for reperfusion. See Villiers, et al, Disease Models and Mechanisms v. 13(11); 2020 Nov. 1.

In the following studies, the MI model mice were treated with a single dose of 4 mg/kg body weight anti-Sdc2 antibody Ab3 or equivalent amount of IgG (as a control) administered intravenously through the tail veins immediately following the reperfusion. The Ab3 antibody is a rabbit anti-mouse polyclonal antibody that binds to the Dep-1 binding region in Sdc2.

### 7.13 Example 13: Treatment with an Anti-Sdc2 Antibody Prevented Post-Myocardial Infarction Heart Failure in a Myocardial Infarction (MI) Mouse Model

The MI model mice were treated with a single dose of anti-Sdc2 antibody administered intravenously through the tail veins immediately following the IR surgery. A control group of mice received IgG instead of anti-Sdc2 antibody. Cardiac functions of mice thus treated were measured 1 day, 7 days, 14 days, and 1 month after the IR surgery.

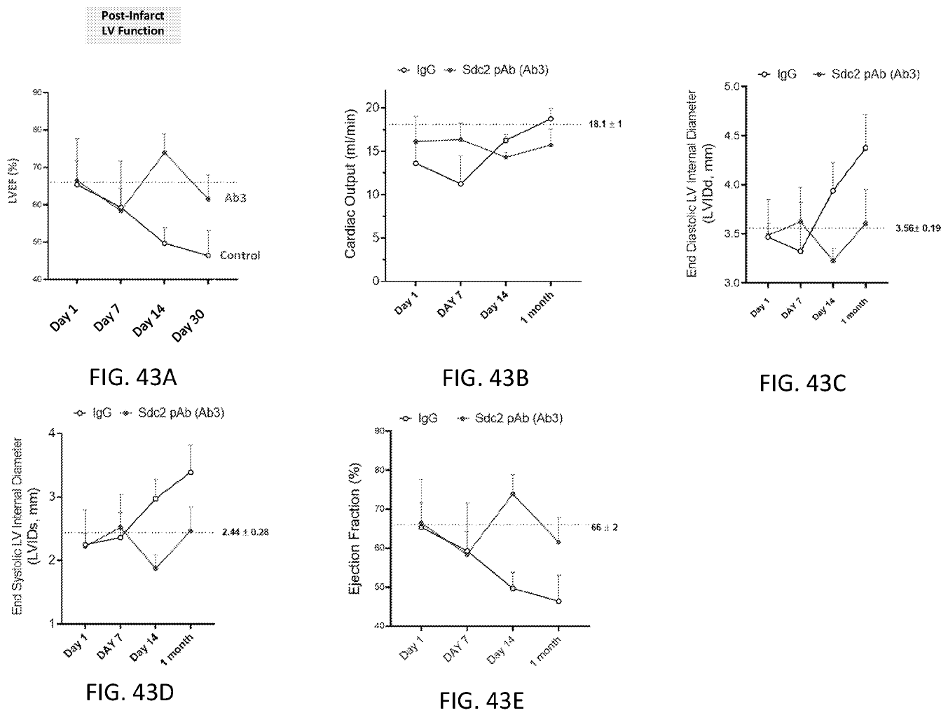

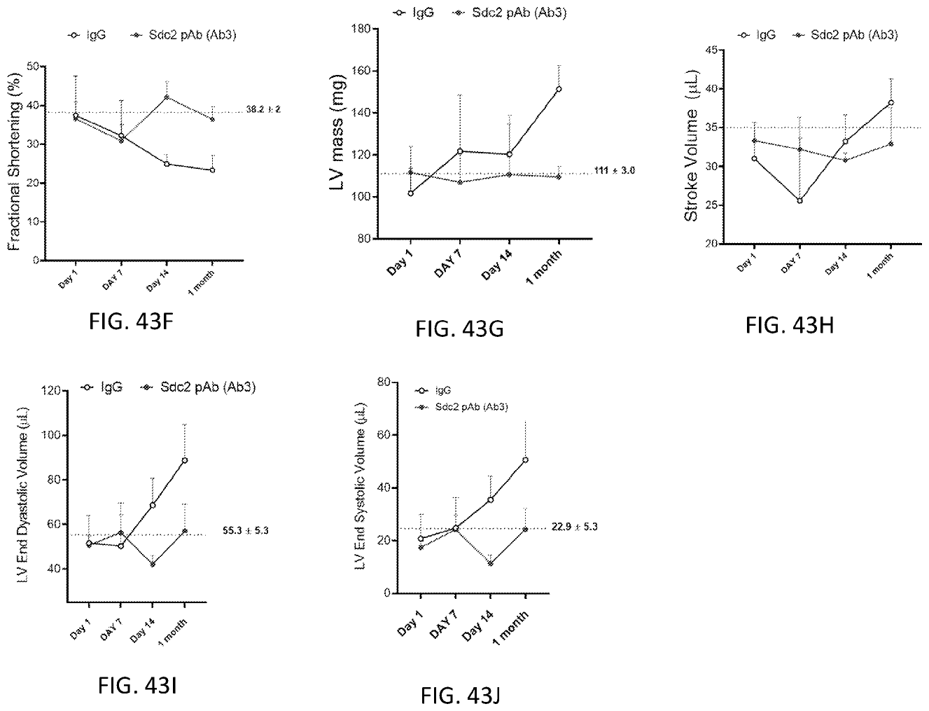

Left ventricular ejection fraction (LVEF), which measures the fraction of chamber volume ejected in systole (stroke volume) in relation to the volume of the blood in the ventricle at the end of diastole (end-diastolic volume), was used to indicate the left ventricular systolic function after the induced MI. As shown in FIG. 43A, mice treated with the polyclonal anti-Sdc2 antibody (Ab3) had LVEF comparable to normal mice without the IR surgery, which were significantly higher than the control group of mice received the IgG.

Cardiac output (CO) is the amount of blood pumped by the heart per minute, and a low CO value is typically associated with and indicates heart failure. As shown in FIG. 43B, MI mice treated with the Ab3 antibody had consistent CO values throughout the one-month observation period after the IR surgery, which values were comparable to that of the normal mice. In contrast, the control group of mice received the IgG had significantly lower CO values on Day 7 after the IR surgery.

LV end-diastolic dimension and LV end-systolic diameter are parameters associated with cardiovascular death, both of which are correlated with LV mass. Typically, subjects having a higher LV mass, LV end-diastolic diameter or LV end-systolic diameter were more prone to sever heart failure or sudden cardiac death. As shown in FIGS. 43C, 43D and 43G, respectively, those three parameters in MI mice treated with Ab3 were comparable to normal mouse during the one-month observation period after the IR surgery, and the measurements were significantly lower than the corresponding values measured from the control group of mice received the IgG.

Stroke volume is the volume of blood pumped out of the LV during each systolic cardiac contraction, which is calculated as the end-diastolic volume minus the end-systolic volume. Ejection fraction is the amount of blood that the heart pumps each time it beats, which is calculated as the stoke volume divided by the end-diastolic volume. As shown in FIGS. 43H-43J, the values of stroke volume, LV end-diastolic volume and LV end-systolic volume in MI mice treated with Ab3 were comparable to the control group of mice that received the IgG. As shown in FIGS. 43I and 43J, both the values of LV end-diastolic volume and LV end-systolic volume in MI mice treated with Ab3 were significantly lower than the control group of mice received the IgG. As shown in FIG. 43H, the stroke volumes of Ab3 group were more consistent than the control group of mice received the IgG. As shown in FIG. 43E, the values of ejection fraction in mice treated with Ab3 were comparable to the value in normal mice and were significantly higher than the control group of mice received the IgG starting Day 14 in the one-month observation period after the IR surgery.

Fractional shortening is calculated by measuring the percentage change in left ventricular diameter during systole. As shown in FIG. 43F, the values of fraction shortening in mice treated with Ab3 were comparable to normal mice and were significantly higher than mice treated with IgG starting on Day 14 in the one-month observation period after the IR surgery.

These data demonstrate that the important measurements of heart failure in Ab3 treated mice were comparable to normal mice without AMI induced by the IR surgery, and Ab3 treatment can prevent the progression of post-myocardial infarction heart failure (as seen in the IgG treated animals in the control group subjected to the IR surgery). The treatment with the Ab3 anti-Sdc2 antibody at the time of AMI reduced significantly the infarct size and resulted in normal cardiac contractile function, prevented post-infarct cardiac remodeling (as indicated by increase of LVEDV and LVESV, increase in LV mass in weeks after AMI), suggesting the prevention into progress into post-AMI heart failure in anti-Sdc2 mAb3-treated animals that were subjected to an AMI.

### 7.14 Example 14. Treatment with an Anti-Sdc2 Antibody Decreased the Infarct Size and Tissue Damage and Inflammation in Heart Tissues of MI Model Mice

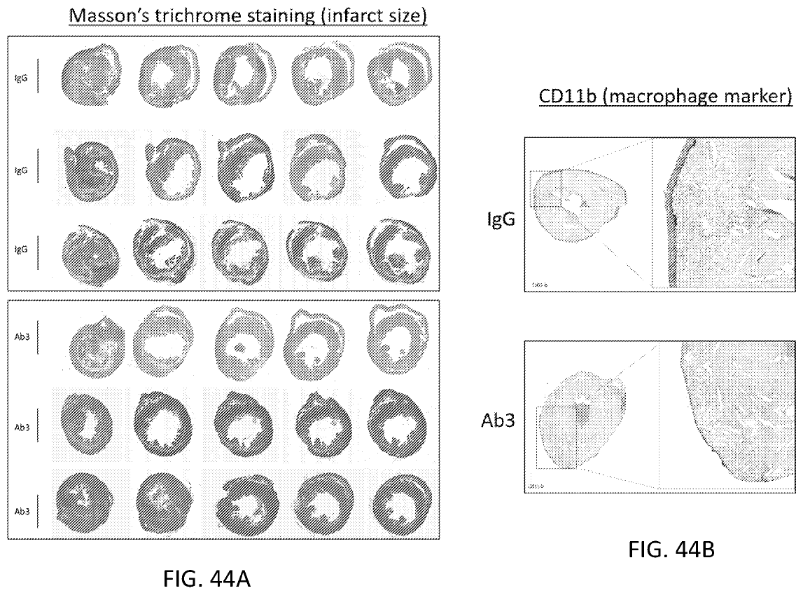

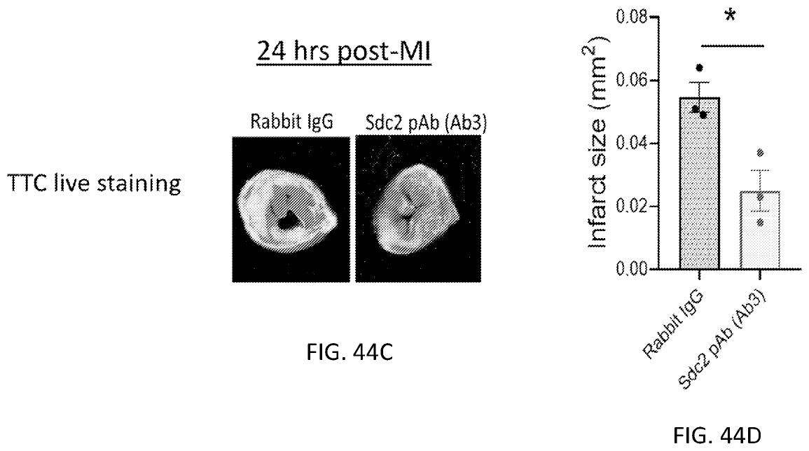

Efficacy of potential treatments for acute myocardial infarction (AMI) is commonly assessed by histological measurement of the infarct size in rodent models. In this study, the MI model were treated with a single dose of anti-Sdc2 antibody (Ab3) administered intravenously through the tail veins immediately following the IR surgery. A control group of mice received IgG instead of anti-Sdc2 antibody. The infarct size and the level of inflammation in the mice were measured at 24 hours (FIGS. 44C and 44D) or one-month (FIGS. 44A and 44B) after the IR surgery.

FIG. 44A shows Masson's trichrome staining images of hearts of MI model mice treated with Ab3 or IgG control, which images were taken one month after the IR surgery. Each row shows a serial of cross-section images of a mouse's heart at different sectional depths. As shown, the heart infarct sizes were visually smaller in the group received Ab3 treatment as compared to the group received the IgG control. The number of macrophages increased in infarcted region after MI due to tissue damage and inflammation. FIG. 44B shows the immunohistochemistry staining visualizing CD11b, a macrophage marker, in the heart tissues of MI mice treated with Ab3 or IgG, which images were taken one month after the IR surgery. As shown, macrophage infiltration in the heart tissues was reduced in the group received Ab3 treatment as compared to the group received the IgG control, indicating the levels of tissue damage and inflammation in the heart tissues were reduced following the anti-Sdc2 treatment.

FIG. 44C shows 2,3,5-Triphenyltetrazolium chloride (TTC) staining images of hearts of MI model mice treated with Ab3 or IgG control, which images were taken 24 hours after the IR surgery and FIG. 44D shows the quantification of the infarct size from this study. As shown, the heart infarct size was significantly smaller (reduced about 56% percent) in the group received Ab3 treatment as compared to the control group of mice received the IgG.

These data demonstrate that beneficial effects of anti-Sdc2 antibody treatment was observable immediately following MI (e.g., within a 24-hour treatment window) and the beneficial effect can be observed after a prolonged period of at least one month.

### 7.15 Example 15: Anti-Sdc2 Antibody Reduced Vascular Permeability and Edema in MI Model Mice

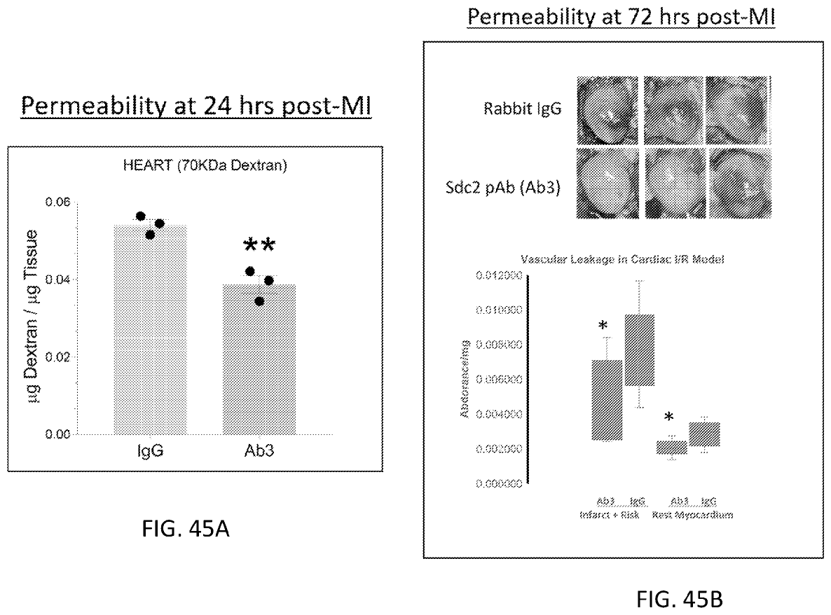

Vascular permeability and leakage in MI model mice treated with a polyclonal anti-Sdc2 antibody (Ab3) were measured using the FITC-dextran permeability assay and Miles assay. Particularly, endothelial permeability was first measured using the FITC-dextran permeability assay 24 hours after the PI surgery. Mice were given an oral gavage of a fluorescently labelled small molecule (FITC-dextran), and after 24 hours, the fluorescence was measured from mice heart tissues. As shown in FIG. 45A, endothelial permeability in mice heart tissues was significantly reduced in the group received Ab3 treatment as compared to the group received the IgG control.

Vascular leakage was further measured using the Miles assay. MI mice were given intravenous injections through tail veins Evans Blue that binds albumin at 70 hours after the IR surgery. Mice were sacrificed at 72 hours after the IR surgery, and immediately perfused with PBS via left ventricle of the heart to remove dye in the blood vessels. The amounts of Evans Blue in heart were calculated according to a standard curve and the weight of the tissue used for extraction. As shown in FIG. 45B, the vascular leakage of mice treated with Ab3 in areas with infarct risk and rest myocardium were significantly lower than those in mice received IgG, measured at 72 hours after the IR surgery.

Vascular permeability is often associated with formation of edema in heart tissues after MI. Moreover, the LV mass was also an indicator of edema formation. As shown in FIG. 43G, the LV mass was significantly lower in MI mice treated with Ab3 as compared to the control group receiving IgG. These data indicate that Ab3 treatment reduced vascular permeability and edema formation at 24 hours and 72 hours after MI. These data also demonstrate that Ab3 treatment prevented compensatory post-infarct cardiomegaly (congestive heart failure, hypertrophy of the remaining functional and viable cardiac muscle to compensate for failing cardiac function), which is a sign of post-infarct cardiac remodeling and is considered a bad prognostic sign.

### 7.16 Example 16: Anti-Sdc2 Antibody Significantly Prevented Infarct Ventricular Tachycardia (VT) Induction Following Induced MI

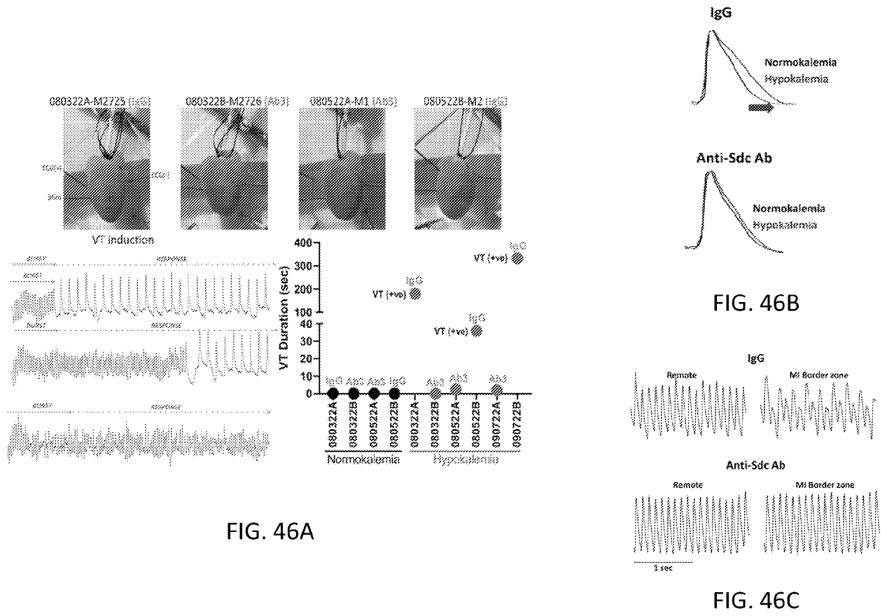

Ventricular tachycardia occurs in 10% to 40% of patients experiencing an episode of AMI. Without being bound by any theory, it is contemplated that infarct scar tissue sets the stage for a reentry circuit that can lead to ventricular tachycardia. To examine the treatment effect of anti-Sdc2 antibody of AMI, in this study, an electrocardiogram (ECG) was performed with programed hypokalemia stimulation on hearts from MI mice treated with Ab3 or received the IgG control. For the group of mice treated with Ab3, longer cycle length of VT was observed following programmed stimulation (see FIG. 46A lower left panel and FIG. 46C), and an increased number of stimuli was needed to induce ventricular tachycardia (VT) or fibrillation (VF) (see FIG. 46A lower left panel), as compared to the control group received IgG.

In this study, duration of the VT under hypokalemic conditions was significantly reduced in Ab3 treated animals, whereas the VT cycle length of the induced VT was longer after programmed stimulation in animals that were treated with the Ab3. (See FIG. 46A lower left and right panel). Slower VT rate was regarded to be more benign and was hemodynamically better tolerated.

AP duration under hypokalemic conditions was normalized in animals treated with mAb3, leading to a more organized slower (monomorphic) VT. The animals in the control group developed a polymorphic VT at a higher frequency (bpm). The induction of VT was more difficult to induce in animals that were treated with mAb (threshold for VT induction was higher) as compared to animals in the control group (see FIGS. 46A, 46B, and 46C).

Furthermore, in both MI border zone and remote region, the mice in Ab3 treated group were more likely to have benign arrhythmias, and less post-infarct ventricular arrhythmias (FIG. 46C). These data demonstrate that anti-Sdc2 antibody treatment can prevent infarct ventricular tachycardia (VT) induction in a subject following an episode of MI.

### 7.17 Example 17: Anti-Sdc2 Antibody could Significantly Prevent Infarct Ventricular Tachycardia (VT) Induction

Following AMI, various inflammatory cytokines are secreted by circulating inflammatory cells and cardiac resident cells, amplifying the pro-inflammatory response to AMI induced by the ischemia-reperfusion injury by moderating the local recruitment of inflammatory cells. Therefore, without being bound by any theory, it is contemplated that the amount of secretion of inflammatory cytokines in hearts can be used as indicators of the severity of the acute ischemia-reperfusion injury.

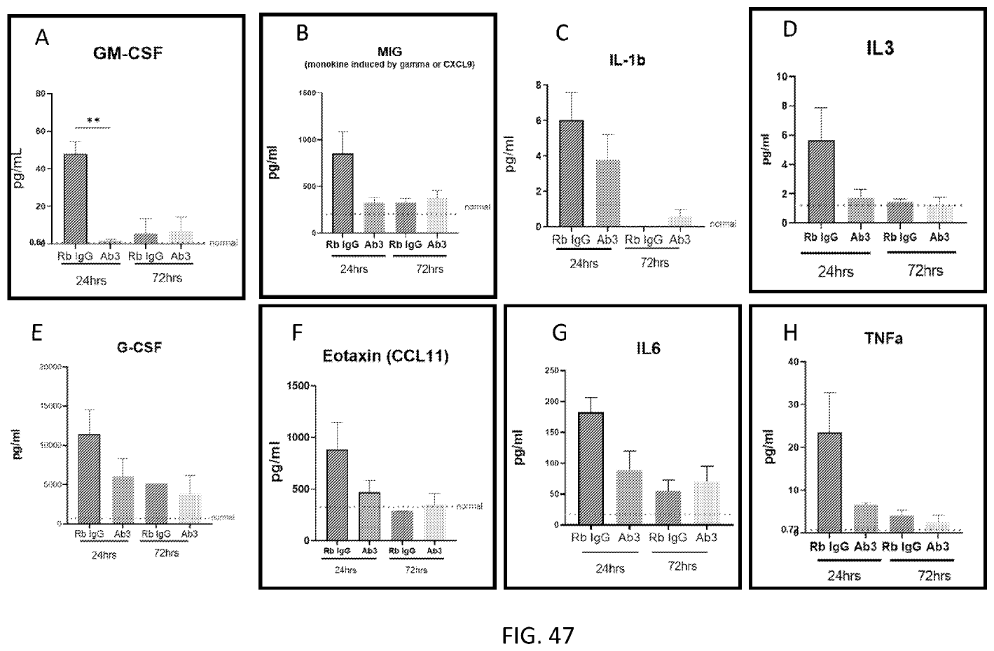

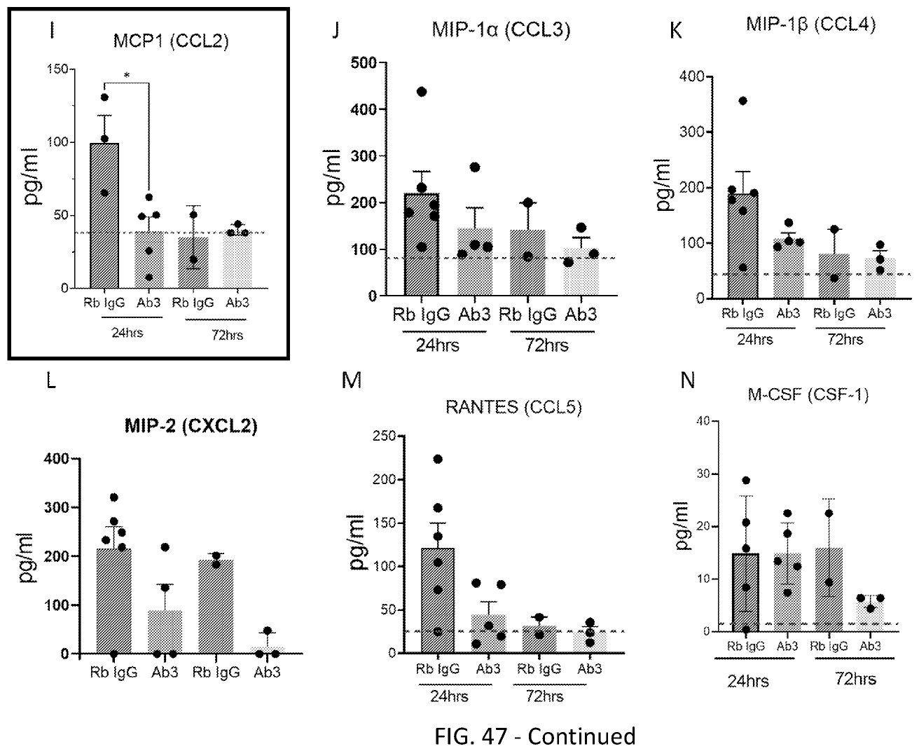

In this study, the plasma concentration of various biomarkers was measured by ELISA 24 hours and 72 hours after the IR surgery. It was observed that, at the 24-hour time point, for 7 of the measured biomarkers, namely GM-CSF, MIG, Eotaxin (CCL11), IL-3, IL-6, TNFα, and MCP1 (CCL2), the concentrations in IgG control group were significantly higher than those in the Ab3 treatment group, and the biomarker levels in the Ab3 treatment group were comparable to the normal level in healthy subjects (FIG. 47). Subsequently, 72 hours after the surgery, cytokines levels in both the Ab3 treatment group and the IgG control group were comparable to the normal level. (FIG. 47). These data indicate that anti-Sdc2 antibody treatment could prevent severe tissue damage resulted from AMI.

### 8. ENUMERATED EMBODIMENTS

The following enumerated, non-limiting embodiments are provided, the numbering of which should not be construed as designating levels of importance.

In a first set of embodiments, provided are the following:

Embodiment 1 provides an antibody or antigen-binding fragment thereof comprising an antigen-binding domain that specifically binds to an epitope of syndecan-2 (Sdc2) protein, wherein the antigen binding domain comprises a heavy chain variable region comprising an amino acid sequence having at least 80% sequence identity to at least one amino acid sequence selected from the group consisting of SEQ ID NOs: 1, 3, 5, 7, 9, 11, 13, 15, 17, 19, 21, and 23 and a light chain variable region comprising an amino acid sequence having at least 80% sequence identity to at least one amino acid selected from the group consisting of SEQ ID NOs: 2, 4, 6, 8, 10, 12, 14, 16, 18, 20, 22, and 24.

Embodiment 2 provides the antibody or antigen-binding fragment thereof of embodiment 1, wherein the heavy chain variable region amino acid sequence is selected from the group consisting of SEQ ID NOs: 1, 3, 5, 7, 9, 11, 13, 15, 17, 19, 21, and 23 and the light chain variable region amino acid sequence is selected from the group consisting of SEQ ID NOs: 2, 4, 6, 8, 10, 12, 14, 16, 18, 20, 22, and 24.

Embodiment 3 provides an antibody or antigen-binding fragment thereof comprising an antigen-binding domain that specifically binds to an epitope of syndecan-2 (Sdc2) protein, wherein the antibody was isolated from a mammal using a strategy of alternating immunization with the extracellular domain of human syndecan-2 (SEQ ID NO: 27) and at least one AG3 (DEP-1 binding) peptide selected from the group consisting of SEQ ID NOs: 28-30 or any combination thereof.

Embodiment 4 provides an antibody or antigen-binding fragment thereof comprising an antigen-binding domain that specifically binds to an epitope of syndecan-2 (Sdc2) protein, wherein the antibody was isolated from a human antibody library using phage display, wherein the antibody was identified after panning with alternating proteins including the extracellular domain of human syndecan-2 is SEQ ID NO: 27 and at least one AG3 (DEP-1 binding) peptide selected from the group consisting of SEQ ID NOs: 28-30 or any combination thereof.

Embodiment 5 provides the antibody or antigen-binding fragment thereof of embodiment 1, wherein the isotype of the antibody or antigen-binding fragment thereof is selected from the group consisting of IgA, IgD, IgE, IgG, and IgM.

Embodiment 6 provides the antibody of antigen-binding fragment thereof of embodiment 1, wherein the isotype of the antibody or antigen-binding fragment thereof is selected from the group consisting of IgG1 and IgG4.

Embodiment 7 provides the antibody or antigen-binding fragment thereof of any one of embodiments 1-4, wherein the antibody or antigen-binding fragment thereof is a selected from the group consisting of a full-length antibody, a Fab, and a single-chain variable fragment (scFv).

Embodiment 8 provides the antibody or antigen-binding fragment thereof of embodiment 6, wherein the antibody or antigen-binding fragment thereof is a full-length antibody.

Embodiment 9 provides the antibody or antigen-binding fragment thereof of embodiment 2, wherein the heavy chain variable region amino acid sequence and the light chain variable region amino acid sequence are respectively selected from the group consisting of SEQ ID NOs: 1 and 2, 7 and 8, 11 and 12, 13 and 14, 15 and 16, and 17 and 18.

Embodiment 10 provides the antibody or antigen-binding fragment thereof of any one of embodiments 1-8, wherein the antibody or antigen-binding fragment thereof is humanized.

Embodiment 11 provides the antibody or antigen-binding fragment thereof of any one of embodiments 1-8, wherein the antibody or antigen-binding fragment thereof is fully-human.

Embodiment 12 provides the antibody or antigen-binding fragment thereof of any one of embodiments 1-10, wherein the Sdc2 protein is selected from the group consisting of human Sdc2, mouse Sdc2, porcine Sdc2, or any combination thereof.

Embodiment 13 provides a single-chain variable fragment (scFv) comprising an antigen-binding domain that specifically binds to an epitope of syndecan-2 (Sdc2) protein, wherein the antigen-binding domain comprises: (i) a heavy chain variable region comprising an amino acid sequence having at least 80% sequence identity to at least one amino acid sequence selected from the group consisting of SEQ ID NOs: 1, 3, 5, 7, 9, 11, 13, 15, 17, 19, 21, and 23; and (ii) a light chain variable region comprising an amino acid sequence having at least 80% sequence identity to at least one amino acid selected from the group consisting of SEQ ID NOs: 2, 4, 6, 8, 10, 12, 14, 16, 18, 20, 22, and 24.

Embodiment 14 provides the scFv of embodiment 12, wherein the Sdc2 protein is selected from the group consisting of human Sdc2, mouse Sdc2, porcine Sdc2, or any combination thereof.

Embodiment 15 provides a full-length antibody comprising an antigen-binding domain that specifically binds to an epitope of syndecan-2 (Sdc2) protein, wherein the antigen-binding domain comprises:
i. a heavy chain variable region comprising an amino acid sequence having at least 80% sequence identity to at least one amino acid sequence selected from the group consisting of SEQ ID NOs: 1, 3, 5, 7, 9, 11, 13, 15, 17, 19, 21, and 23; and
ii. a light chain variable region comprising an amino acid sequence having at least 80% sequence identity to at least one amino acid selected from the group consisting of SEQ ID NOs: 2, 4, 6, 8, 10, 12, 14, 16, 18, 20, 22, and 24.

Embodiment 16 provides the full-length antibody of embodiment 15, wherein the Sdc2 protein is selected from the group consisting of human Sdc2, mouse Sdc2, porcine Sdc2, or any combination thereof.

Embodiment 17 provides an isolated nucleic acid encoding the scFv or full-length antibody or antigen-binding fragment thereof of any preceding embodiment.

Embodiment 18 provides an isolated nucleic acid encoding an antibody or antigen-binding fragment thereof comprising an antigen-binding domain that specifically binds an epitope of syndecan-2 (Sdc2) protein, wherein the antigen-binding domain comprises: (i) a heavy chain variable region comprising an amino acid sequence having at least 80% sequence identity to at least one amino acid sequence selected from the group consisting of SEQ ID NOs: 1, 3, 5, 7, 9, 11, 13, 15, 17, 19, 21, and 23; and (ii) a light chain variable region comprising an amino acid sequence having at least 80% sequence identity to at least one amino acid selected from the group consisting of SEQ ID NOs: 2, 4, 6, 8, 10, 12, 14, 16, 18, 20, 22, and 24.

Embodiment 19 provides the isolated nucleic acid of embodiment 18, wherein the antibody or antigen-binding fragment thereof is a selected from the group consisting of a full-length antibody, a Fab, and a single-chain variable fragment (scFv).

Embodiment 20 provides the isolated nucleic acid of embodiment 19, wherein the antibody or antigen-binding fragment thereof is a full-length antibody.

Embodiment 21 provides the isolated nucleic acid of embodiment 18, wherein the antibody or antigen-binding fragment thereof is humanized.

Embodiment 22 provides the isolated nucleic acid of embodiment 18, wherein the antibody or antigen-binding fragment thereof is fully-human.

Embodiment 23 provides the isolated nucleic acid of embodiment 18, wherein the Sdc2 protein is selected from the group consisting of human Sdc2, mouse Sdc2, porcine Sdc2, or any combination thereof.

Embodiment 24 provides an isolated nucleic acid encoding a single-chain variable fragment (scFv) comprising an antigen-binding domain that specifically binds an epitope of syndecan-2 (Sdc2) protein, wherein the antigen-binding domain comprises: (i) a heavy chain variable region comprising an amino acid sequence having at least 80% sequence identity to at least one amino acid sequence selected from the group consisting of SEQ ID NOs: 1, 3, 5, 7, 9, 11, 13, 15, 17, 19, 21, and 23; and (ii) a light chain variable region comprising an amino acid sequence having at least 80% sequence identity to at least one amino acid selected from the group consisting of SEQ ID NOs: 2, 4, 6, 8, 10, 12, 14, 16, 18, 20, 22, and 24.

Embodiment 25 provides a vector comprising the isolated nucleic acid of any one of embodiments 17-24.

Embodiment 26 provides the vector of embodiment 25, wherein the vector is an expression vector.

Embodiment 27 provides the vector of any one of embodiments 25-26, wherein the vector is selected from the group consisting of a DNA vector, an RNA vector, a plasmid, a lentiviral vector, an adenoviral vector, an adeno-associated vector, and a retroviral vector.

Embodiment 28 provides a host cell comprising the vector of any one of embodiments 23-25.

Embodiment 29 provides a pharmacological composition comprising the full-length antibody or scFv of any one of embodiments 1-16 and a pharmaceutically acceptable carrier.

Embodiment 30 provides a method of treating a syndecan-2 associated disease in a subject in need thereof, the method comprising administering to the subject an effective amount of an anti-syndecan-2 antibody, wherein the antigen binding domain comprises a heavy chain variable region comprising an amino acid sequence having at least 80% sequence identity to at least one amino acid sequence selected from the group consisting of SEQ ID NOs: 1, 3, 5, 7, 9, 11, 13, 15, 17, 19, 21, and 23 and a light chain variable region comprising an amino acid sequence having at least 80% sequence identity to at least one amino acid selected from the group consisting of SEQ ID NOs: 2, 4, 6, 8, 10, 12, 14, 16, 18, 20, 22, and 24.

Embodiment 31 provides the method of embodiment 30, wherein the syndecan-2 associated disease is acute respiratory distress syndrome (ARDS), hemorrhagic stroke, ischemic stroke, a neurological disease in which the blood brain barrier (BBB) is altered or disrupted, neovascular eye disease, cardiovascular disease with a component of vascular hyperpermeability, recovery after blunt trauma injuries, peripheral vascular disease, lymphedema and inflammation-associated edema, inflammatory disease, or cancer.

Embodiment 32 provides the method of embodiment 30, wherein the syndecan-2 associated disease is coronavirus disease 2019-induced ARDS (COVID-19-induced ARDS).

Embodiment 33 provides the method of embodiment 30, wherein the subject is a mammal.

Embodiment 34 provides the method of embodiment 30, wherein the subject is a human.

Embodiment 35 provides the method of embodiment 30, wherein the anti-syndecan-2 antibody is administered in a dosage form further comprising at least one pharmaceutically acceptable carrier.

Embodiment 36 provides a method of treating a vascular leak-associated disease in a subject in need thereof, comprising administering to the subject an effective amount of an anti-syndecan-2 antibody, wherein the antigen binding domain comprises a heavy chain variable region comprising an amino acid sequence having at least 80% sequence identity to at least one amino acid sequence selected from the group consisting of SEQ ID NOs: 1, 3, 5, 7, 9, 11, 13, 15, 17, 19, 21, and 23 and a light chain variable region comprising an amino acid sequence having at least 80% sequence identity to at least one amino acid selected from the group consisting of SEQ ID NOs: 2, 4, 6, 8, 10, 12, 14, 16, 18, 20, 22, and 24.

Embodiment 37 provides the method of embodiment 36, wherein the anti-syndecan-2 antibody inhibits syndecan-2 signaling.

Embodiment 38 provides the method of embodiment 36, wherein the anti-syndecan-2 antibody inhibits Dep-1 internalization.

Embodiment 39 provides the method of embodiment 36, wherein the anti-syndecan-2 antibody enhances Dep-1 internalization.

Embodiment 40 provides the method of embodiment 36, wherein the vascular leak-associated disease is a cancer.

Embodiment 41 provides the method of embodiment 36, wherein the vascular leak-associated disease is a cardiovascular disease.

Embodiment 42 provides the method of embodiment 41, wherein the cardiovascular disease is myocardial infarction or congestive heart failure.

Embodiment 43 provides the method of embodiment 36, wherein the vascular leak-associated disease is a neovascular eye disease.

Embodiment 44 provides the method of embodiment 43, wherein the neovascular eye disease is selected from the group consisting of AMD and diabetic retinopathy.

Embodiment 45 provides the method of embodiment 35, wherein the vascular leak-associated disease is acute respiratory distress syndrome (ARDS).

Embodiment 46 provides the method of embodiment 45, wherein the ARDS is associated with a viral infection.

Embodiment 47 provides the method of embodiment 46, wherein the viral infection is caused by SARS-CoV-2.

Embodiment 48 provides the method of embodiment 36, wherein the vascular leak-associated disease is an inflammatory disease.

Embodiment 49 provides the method of embodiment 48, wherein the inflammatory disease is selected from the group consisting of systemic lupus erythematosus and rheumatoid arthritis.

Embodiment 50 provides the method of embodiment 36, wherein the treatment accelerates the natural healing response.

Embodiment 51 provides the method of embodiment 36, wherein the vascular leak-associated disease is stroke.

Embodiment 52 provides the method of embodiment 51, wherein the stroke is ischemic stroke.

Embodiment 53 provides the method of embodiment 51, wherein the stroke is hemorrhagic stroke.

Embodiment 54 provides the method of embodiment 36, wherein the vascular leak-associated disease is disease that are accompanied by an alteration and/or disruption of the blood brain barrier (BBB).

Embodiment 55 provides the method of embodiment 51, wherein the disruption of the blood brain barrier is associated with a disease selected from the group consisting of Parkinson's Diseases, Alzheimer's disease, Huntington's Disease, peripheral neuropathies, traumatic brain injury, epilepsy and multiple sclerosis

Embodiment 56 provides the method of embodiment 36, wherein the vascular leak-associated disease is complications due to blunt trauma injuries.

Embodiment 57 provides the method of embodiment 55, wherein the blunt trauma injury is a traumatic brain injury.

Embodiment 58 provides the method of embodiment 55, wherein the blunt trauma injury is a battlefield injury.

Embodiment 59 provides the method of embodiment 36, wherein the vascular leak-associated disease is peripheral vascular disease.

Embodiment 60 provides the method of embodiment 59, wherein the peripheral vascular disease is associated with a disease selected from the group consisting of lymphedema, POEMS Syndrome, pediatric capillary leak syndrome, adult capillary leak syndrome, and hydrocephalus.

Embodiment 61 provides a human or humanized antibody or antigen-binding fragment thereof comprising an antigen-binding domain that specifically binds to an epitope of syndecan-2 (Sdc2) protein, wherein the antibody or antigen-binding fragment thereof binds to the extracellular domain of Sdc2, and wherein the binding of the antibody or antigen-binding fragment thereof alters Sdc2 signaling, and wherein the binding of the Sdc2 extracellular domain inhibits vascular permeability.

Embodiment 62 provides the antibody or antigen binding fragment thereof of embodiment 59, wherein the antibody or antigen-binding fragment thereof binds to the Dep-1 binding site of the extracellular domain of Sdc2.

Embodiment 63 provides the antibody or antigen binding fragment thereof of embodiment 59, wherein the antibody or antigen-binding fragment thereof binds outside of the Dep-1 binding site of the extracellular domain of Sdc2.

Embodiment 64 provides the antibody or antigen binding fragment thereof of embodiment 61, wherein the binding of the antibody or antigen-binding fragment thereof to Sdc2 results in increased Dep-1 internalization.

Embodiment 65 provides the antibody or antigen binding fragment thereof of embodiment 61, wherein the binding of the antibody or antigen-binding fragment thereof to Sdc2 blocks Dep-1 internalization.

Embodiment 66 provides a method of treating a syndecan-2 associated disease in a subject in need thereof, the method comprising administering to the subject an effective amount of a human or humanized anti-syndecan-2 antibody, wherein the antibody or antigen-binding fragment thereof binds to the extracellular domain of Sdc2, and wherein the binding of the antibody or antigen-binding fragment thereof alters syndecan-2 signaling, and wherein the binding of the Sdc2 extracellular domain inhibits vascular permeability.

Embodiment 67 provides the method of embodiment 66, wherein the syndecan-2 associated disease is acute respiratory distress syndrome (ARDS), hemorrhagic stroke, ischemic stroke, a neurological disease in which the blood brain barrier (BBB) is altered or disrupted, neovascular eye disease, cardiovascular disease with a component of vascular hyperpermeability, recovery after blunt trauma injuries, peripheral vascular disease, lymphedema and inflammation-associated edema, inflammatory disease, or cancer.

Embodiment 68 provides the method of embodiment 66, wherein the syndecan-2 associated disease is coronavirus disease 2019-induced ARDS (COVID-19-induced ARDS).

Embodiment 69 provides the method of embodiment 66, wherein the subject is a mammal.

Embodiment 70 provides the method of embodiment 66, wherein the subject is a human.

Embodiment 71 provides the method of embodiment 66, wherein the anti-syndecan-2 antibody is administered in a dosage form further comprising at least one pharmaceutically acceptable carrier.

Embodiment 72 provides a method of treating a vascular leak-associated disease in a subject in need thereof, comprising administering to the subject an effective amount of a human or humanized anti-syndecan-2 antibody, wherein the antibody or antigen-binding fragment thereof binds to the extracellular domain of Sdc2, and wherein the binding of the antibody or antigen-binding fragment thereof alters syndecan-2 signaling, and wherein the binding of the Sdc2 extracellular domain inhibits vascular permeability.

Embodiment 73 provides the method of embodiment 72, wherein the vascular leak-associated disease is a cancer.

Embodiment 74 provides the method of embodiment 72, wherein the vascular leak-associated disease is a cardiovascular disease.

Embodiment 75 provides the method of embodiment 74, wherein the cardiovascular disease is myocardial infarction or congestive heart failure.

Embodiment 76 provides the method of embodiment 72, wherein the vascular leak-associated disease is a neovascular eye disease.

Embodiment 77 provides the method of embodiment 76, wherein the neovascular eye disease is selected from the group consisting of AMD and diabetic retinopathy.

Embodiment 78 provides the method of embodiment 72, wherein the vascular leak-associated disease is acute respiratory distress syndrome (ARDS).

Embodiment 79 provides the method of embodiment 78, wherein the ARDS is associated with a viral infection.

Embodiment 80 provides the method of embodiment 79, wherein the viral infection is caused by SARS-CoV-2.

Embodiment 81 provides the method of embodiment 72, wherein the vascular leak-associated disease is an inflammatory disease.

Embodiment 82 provides the method of embodiment 81, wherein the inflammatory disease is selected from the group consisting of systemic lupus erythematosus and rheumatoid arthritis.

Embodiment 83 provides the method of embodiment 72, wherein the treatment accelerates the natural healing response.

Embodiment 84 provides the method of embodiment 72, wherein the vascular leak-associated disease is stroke.

Embodiment 85 provides the method of embodiment 84, wherein the stroke is ischemic stroke.

Embodiment 86 provides the method of embodiment 84, wherein the stroke is hemorrhagic stroke.

Embodiment 87 provides the method of embodiment 72, wherein the vascular leak-associated disease is disease that are accompanied by an alteration and/or disruption of the blood brain barrier (BBB).

Embodiment 88 provides the method of embodiment 87, wherein the disruption of the blood brain barrier is associated with a disease selected from the group consisting of Parkinson's Diseases, Alzheimer's disease, Huntington's Disease, peripheral neuropathies, traumatic brain injury, epilepsy and multiple sclerosis.

Embodiment 89 provides the method of embodiment 72, wherein the vascular leak-associated disease is complications due to blunt trauma injuries.

Embodiment 90 provides the method of embodiment 89, wherein the blunt trauma injury is a traumatic brain injury.

Embodiment 91 provides the method of embodiment 89, wherein the blunt trauma injury is a battlefield injury.

Embodiment 92 provide the method of embodiment 72, wherein the vascular leak-associated disease is peripheral vascular disease.

Embodiment 93 provides the method of embodiment 92, wherein the peripheral vascular disease is associated with a disease selected from the group consisting of lymphedema, POEMS Syndrome, pediatric capillary leak syndrome, adult capillary leak syndrome, and hydrocephalus.

Embodiment 94 provides an antibody that binds Sdc2 comprising: (a) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:61; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:62; (b) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 95; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:96; (c) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 129; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 130; (d) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 163; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 164; (e) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:197; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 198; (f) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:231; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:232; (g) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:265; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:266; (h) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:299; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:300; (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 333; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:334; (j) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:367; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:368; (k) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:401; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:402; or (l) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:435; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 436.

Embodiment 95 provides the antibody of embodiment 93, wherein (i) the VH CDR1, VH CDR2, VH CDR3, VL CDR1, VL CDR2, and VL CDR3 amino acid sequences are according to the Kabat numbering system; (ii) the VH CDR1, VH CDR2, VH CDR3, VL CDR1, VL CDR2, and VL CDR3 amino acid sequences are according to the Chothia numbering system; (iii) the VH CDR1, VH CDR2, VH CDR3, VL CDR1, VL CDR2, and VL CDR3 amino acid sequences are according to the AbM numbering system; (iv) the VH CDR1, VH CDR2, VH CDR3, VL CDR1, VL CDR2, and VL CDR3 amino acid sequences are according to the Contact numbering system; or (v) the VH CDR1, VH CDR2, VH CDR3, VL CDR1, VL CDR2, and VL CDR3 amino acid sequences are according to the IMGT numbering system.

Embodiment 96 provides the antibody of embodiment 94 or 95, comprising: (I) (A) a VH comprising a VH CDR1, a VH CDR2, and a VH CDR3 having an amino acid sequence of SEQ ID NOs: 31, 32 and 33, respectively; and (ii) a VL comprising a VL CDR1, a VL CDR2, and a VL CDR3 having an amino acid sequence of SEQ ID NOs: 34, 35 and 36, respectively; (B) a VH comprising a VH CDR1, a VH CDR2, and a VH CDR3 having an amino acid sequence of SEQ ID NOs: 37, 38 and 39, respectively; and (ii) a VL comprising a VL CDR1, a VL CDR2, and a VL CDR3 having an amino acid sequence of SEQ ID NOs: 40, 41 and 42, respectively; (C) a VH comprising a VH CDR1, a VH CDR2, and a VH CDR3 having an amino acid sequence of SEQ ID NOs: 43, 44 and 45, respectively; and (ii) a VL comprising a VL CDR1, a VL CDR2, and a VL CDR3 having an amino acid sequence of SEQ ID NOs: 46, 47 and 48, respectively; (D) a VH comprising a VH CDR1, a VH CDR2, and a VH CDR3 having an amino acid sequence of SEQ ID NOs: 49, 50 and 51, respectively; and (ii) a VL comprising a VL CDR1, a VL CDR2, and a VL CDR3 having an amino acid sequence of SEQ ID NOs: 52, 53 and 54, respectively; or (E) a VH comprising a VH CDR1, a VH CDR2, and a VH CDR3 having an amino acid sequence of SEQ ID NOs: 55, 56 and 57, respectively; and (ii) a VL comprising a VL CDR1, a VL CDR2, and a VL CDR3 having an amino acid sequence of SEQ ID NOs: 58, 59 and 60, respectively; (II) (A) a VH comprising a VH CDR1, a VH CDR2, and a VH CDR3 having an amino acid sequence of SEQ ID NOs: 65, 66 and 67, respectively; and (ii) a VL comprising a VL CDR1, a VL CDR2, and a VL CDR3 having an amino acid sequence of SEQ ID NOs: 68, 89 and 70, respectively; (B) a VH comprising a VH CDR1, a VH CDR2, and a VH CDR3 having an amino acid sequence of SEQ ID NOs: 71, 72 and 73, respectively; and (ii) a VL comprising a VL CDR1, a VL CDR2, and a VL CDR3 having an amino acid sequence of SEQ ID NOs: 74, 75 and 76, respectively; (C) a VH comprising a VH CDR1, a VH CDR2, and a VH CDR3 having an amino acid sequence of SEQ ID NOs: 77, 78 and 79, respectively; and (ii) a VL comprising a VL CDR1, a VL CDR2, and a VL CDR3 having an amino acid sequence of SEQ ID NOs: 80, 81 and 82, respectively; (D) a VH comprising a VH CDR1, a VH CDR2, and a VH CDR3 having an amino acid sequence of SEQ ID NOs: 83, 84 and 85, respectively; and (ii) a VL comprising a VL CDR1, a VL CDR2, and a VL CDR3 having an amino acid sequence of SEQ ID NOs: 86, 87 and 88, respectively; or (E) a VH comprising a VH CDR1, a VH CDR2, and a VH CDR3 having an amino acid sequence of SEQ ID NOs: 89, 90 and 91, respectively; and (ii) a VL comprising a VL CDR1, a VL CDR2, and a VL CDR3 having an amino acid sequence of SEQ ID NOs: 92, 93 and 94, respectively; (III.) (A) a VH comprising a VH CDR1, a VH CDR2, and a VH CDR3 having an amino acid sequence of SEQ ID NOs: 99, 100 and 101, respectively; and (ii) a VL comprising a VL CDR1, a VL CDR2, and a VL CDR3 having an amino acid sequence of SEQ ID NOs: 102,103 and 104, respectively; (B) a VH comprising a VH CDR1, a VH CDR2, and a VH CDR3 having an amino acid sequence of SEQ ID NOs: 105, 106 and 107, respectively; and (ii) a VL comprising a VL CDR1, a VL CDR2, and a VL CDR3 having an amino acid sequence of SEQ ID NOs: 108, 109 and 110, respectively; (C) a VH comprising a VH CDR1, a VH CDR2, and a VH CDR3 having an amino acid sequence of SEQ ID NOs: 111, 112 and 113, respectively; and (ii) a VL comprising a VL CDR1, a VL CDR2, and a VL CDR3 having an amino acid sequence of SEQ ID NOs: 114, 115 and 116, respectively; (D) a VH comprising a VH CDR1, a VH CDR2, and a VH CDR3 having an amino acid sequence of SEQ ID NOs: 117, 118 and 119, respectively; and (ii) a VL comprising a VL CDR1, a VL CDR2, and a VL CDR3 having an amino acid sequence of SEQ ID NOs: 120, 121 and 122, respectively; or (E) a VH comprising a VH CDR1, a VH CDR2, and a VH CDR3 having an amino acid sequence of SEQ ID NOs: 123, 124 and 125, respectively; and (ii) a VL comprising a VL CDR1, a VL CDR2, and a VL CDR3 having an amino acid sequence of SEQ ID NOs: 126, 127 and 128, respectively; (IV.) (A) a VH comprising a VH CDR1, a VH CDR2, and a VH CDR3 having an amino acid sequence of SEQ ID NOs: 133, 134 and 135, respectively; and (ii) a VL comprising a VL CDR1, a VL CDR2, and a VL CDR3 having an amino acid sequence of SEQ ID NOs: 136, 137 and 138, respectively; (B) a VH comprising a VH CDR1, a VH CDR2, and a VH CDR3 having an amino acid sequence of SEQ ID NOs: 139, 140 and 141, respectively; and (ii) a VL comprising a VL CDR1, a VL CDR2, and a VL CDR3 having an amino acid sequence of SEQ ID NOs: 142, 143 and 144, respectively; (C) a VH comprising a VH CDR1, a VH CDR2, and a VH CDR3 having an amino acid sequence of SEQ ID NOs: 145, 146 and 147, respectively; and (ii) a VL comprising a VL CDR1, a VL CDR2, and a VL CDR3 having an amino acid sequence of SEQ ID NOs: 148, 149 and 150, respectively; (D) a VH comprising a VH CDR1, a VH CDR2, and a VH CDR3 having an amino acid sequence of SEQ ID NOs: 151, 152 and 153, respectively; and (ii) a VL comprising a VL CDR1, a VL CDR2, and a VL CDR3 having an amino acid sequence of SEQ ID NOs: 154, 155 and 156, respectively; or (E) a VH comprising a VH CDR1, a VH CDR2, and a VH CDR3 having an amino acid sequence of SEQ ID NOs: 157, 158 and 159, respectively; and (ii) a VL comprising a VL CDR1, a VL CDR2, and a VL CDR3 having an amino acid sequence of SEQ ID NOs: 160, 161 and 162, respectively; (V.) (A) a VH comprising a VH CDR1, a VH CDR2, and a VH CDR3 having an amino acid sequence of SEQ ID NOs: 167, 168 and 169, respectively; and (ii) a VL comprising a VL CDR1, a VL CDR2, and a VL CDR3 having an amino acid sequence of SEQ ID NOs: 170, 171 and 172, respectively; (B) a VH comprising a VH CDR1, a VH CDR2, and a VH CDR3 having an amino acid sequence of SEQ ID NOs: 173, 174 and 175, respectively; and (ii) a VL comprising a VL CDR1, a VL CDR2, and a VL CDR3 having an amino acid sequence of SEQ ID NOs: 176, 177 and 178, respectively; (C) a VH comprising a VH CDR1, a VH CDR2, and a VH CDR3 having an amino acid sequence of SEQ ID NOs: 179, 180 and 181, respectively; and (ii) a VL comprising a VL CDR1, a VL CDR2, and a VL CDR3 having an amino acid sequence of SEQ ID NOs: 182, 183 and 184, respectively; (D) a VH comprising a VH CDR1, a VH CDR2, and a VH CDR3 having an amino acid sequence of SEQ ID NOs: 185, 186 and 187, respectively; and (ii) a VL comprising a VL CDR1, a VL CDR2, and a VL CDR3 having an amino acid sequence of SEQ ID NOs: 188, 189 and 190, respectively; or (E) a VH comprising a VH CDR1, a VH CDR2, and a VH CDR3 having an amino acid sequence of SEQ ID NOs: 191, 192 and 193, respectively; and (ii) a VL comprising a VL CDR1, a VL CDR2, and a VL CDR3 having an amino acid sequence of SEQ ID NOs: 194, 195 and 196, respectively; (VI.) (A) a VH comprising a VH CDR1, a VH CDR2, and a VH CDR3 having an amino acid sequence of SEQ ID NOs: 201, 202 and 203, respectively; and (ii) a VL comprising a VL CDR1, a VL CDR2, and a VL CDR3 having an amino acid sequence of SEQ ID NOs: 204, 205 and 206, respectively; (B) a VH comprising a VH CDR1, a VH CDR2, and a VH CDR3 having an amino acid sequence of SEQ ID NOs: 207, 208 and 209, respectively; and (ii) a VL comprising a VL CDR1, a VL CDR2, and a VL CDR3 having an amino acid sequence of SEQ ID NOs: 210, 211 and 212, respectively; (C) a VH comprising a VH CDR1, a VH CDR2, and a VH CDR3 having an amino acid sequence of SEQ ID NOs: 213, 214 and 215, respectively; and (ii) a VL comprising a VL CDR1, a VL CDR2, and a VL CDR3 having an amino acid sequence of SEQ ID NOs: 216, 217 and 218, respectively; (D) a VH comprising a VH CDR1, a VH CDR2, and a VH CDR3 having an amino acid sequence of SEQ ID NOs: 219, 220 and 221, respectively; and (ii) a VL comprising a VL CDR1, a VL CDR2, and a VL CDR3 having an amino acid sequence of SEQ ID NOs: 222, 223 and 224, respectively; or (E) a VH comprising a VH CDR1, a VH CDR2, and a VH CDR3 having an amino acid sequence of SEQ ID NOs: 225, 226 and 227, respectively; and (ii) a VL comprising a VL CDR1, a VL CDR2, and a VL CDR3 having an amino acid sequence of SEQ ID NOs: 228, 229 and 230, respectively; (VII.) (A) a VH comprising a VH CDR1, a VH CDR2, and a VH CDR3 having an amino acid sequence of SEQ ID NOs: 235, 236 and 237, respectively; and (ii) a VL comprising a VL CDR1, a VL CDR2, and a VL CDR3 having an amino acid sequence of SEQ ID NOs: 238, 239 and 240, respectively; (B) a VH comprising a VH CDR1, a VH CDR2, and a VH CDR3 having an amino acid sequence of SEQ ID NOs: 241, 242 and 243, respectively; and (ii) a VL comprising a VL CDR1, a VL CDR2, and a VL CDR3 having an amino acid sequence of SEQ ID NOs: 244, 245 and 246, respectively; (C) a VH comprising a VH CDR1, a VH CDR2, and a VH CDR3 having an amino acid sequence of SEQ ID NOs: 247, 248 and 249, respectively; and (ii) a VL comprising a VL CDR1, a VL CDR2, and a VL CDR3 having an amino acid sequence of SEQ ID NOs: 250, 251 and 252, respectively; (D) a VH comprising a VH CDR1, a VH CDR2, and a VH CDR3 having an amino acid sequence of SEQ ID NOs: 253, 254 and 255, respectively; and (ii) a VL comprising a VL CDR1, a VL CDR2, and a VL CDR3 having an amino acid sequence of SEQ ID NOs: 256, 257 and 258, respectively; or (E) a VH comprising a VH CDR1, a VH CDR2, and a VH CDR3 having an amino acid sequence of SEQ ID NOs: 259, 260 and 261, respectively; and (ii) a VL comprising a VL CDR1, a VL CDR2, and a VL CDR3 having an amino acid sequence of SEQ ID NOs: 262, 263 and 264, respectively; (VIII.) (A) a VH comprising a VH CDR1, a VH CDR2, and a VH CDR3 having an amino acid sequence of SEQ ID NOs: 269, 270 and 271, respectively; and (ii) a VL comprising a VL CDR1, a VL CDR2, and a VL CDR3 having an amino acid sequence of SEQ ID NOs: 272, 273 and 274, respectively; (B) a VH comprising a VH CDR1, a VH CDR2, and a VH CDR3 having an amino acid sequence of SEQ ID NOs: 275, 276 and 277, respectively; and (ii) a VL comprising a VL CDR1, a VL CDR2, and a VL CDR3 having an amino acid sequence of SEQ ID NOs: 278, 279 and 280, respectively; (C) a VH comprising a VH CDR1, a VH CDR2, and a VH CDR3 having an amino acid sequence of SEQ ID NOs: 281, 282 and 283, respectively; and (ii) a VL comprising a VL CDR1, a VL CDR2, and a VL CDR3 having an amino acid sequence of SEQ ID NOs: 284, 285 and 286, respectively; (D) a VH comprising a VH CDR1, a VH CDR2, and a VH CDR3 having an amino acid sequence of SEQ ID NOs: 287, 288 and 289, respectively; and (ii) a VL comprising a VL CDR1, a VL CDR2, and a VL CDR3 having an amino acid sequence of SEQ ID NOs: 290, 292 and 292, respectively; or (E) a VH comprising a VH CDR1, a VH CDR2, and a VH CDR3 having an amino acid sequence of SEQ ID NOs: 293, 294 and 295, respectively; and (ii) a VL comprising a VL CDR1, a VL CDR2, and a VL CDR3 having an amino acid sequence of SEQ ID NOs: 296, 297 and 298, respectively; (IX). (A) a VH comprising a VH CDR1, a VH CDR2, and a VH CDR3 having an amino acid sequence of SEQ ID NOs: 303, 304 and 305, respectively; and (ii) a VL comprising a VL CDR1, a VL CDR2, and a VL CDR3 having an amino acid sequence of SEQ ID NOs: 306, 307 and 308, respectively; (B) a VH comprising a VH CDR1, a VH CDR2, and a VH CDR3 having an amino acid sequence of SEQ ID NOs: 309, 310 and 311, respectively; and (ii) a VL comprising a VL CDR1, a VL CDR2, and a VL CDR3 having an amino acid sequence of CDR3 having an amino acid sequence of SEQ ID NOs: 315, 316 and 317, respectively; and (ii) a VL comprising a VL CDR1, a VL CDR2, and a VL CDR3 having an amino acid sequence of SEQ ID NOs: 318, 319 and 320, respectively; (D) a VH comprising a VH CDR1, a VH CDR2, and a VH CDR3 having an amino acid sequence of SEQ ID NOs: 321, 322 and 323, respectively; and (ii) a VL comprising a VL CDR1, a VL CDR2, and a VL CDR3 having an amino acid sequence of SEQ ID NOs: 324, 325 and 326, respectively; or (E) a VH comprising a VH CDR1, a VH CDR2, and a VH CDR3 having an amino acid sequence of SEQ ID NOs: 327, 328 and 329, respectively; and (ii) a VL comprising a VL CDR1, a VL CDR2, and a VL CDR3 having an amino acid sequence of SEQ ID NOs: 330, 331 and 332, respectively; (X.) (A) a VH comprising a VH CDR1, a VH CDR2, and a VH CDR3 having an amino acid sequence of SEQ ID NOs: 337, 338 and 339, respectively; and (ii) a VL comprising a VL CDR1, a VL CDR2, and a VL CDR3 having an amino acid sequence of SEQ ID NOs: 340, 341 and 342, respectively; (B) a VH comprising a VH CDR1, a VH CDR2, and a VH CDR3 having an amino acid sequence of SEQ ID NOs: 343, 344 and 345, respectively; and (ii) a VL comprising a VL CDR1, a VL CDR2, and a VL CDR3 having an amino acid sequence of SEQ ID NOs: 346, 347 and 348, respectively; (C) a VH comprising a VH CDR1, a VH CDR2, and a VH CDR3 having an amino acid sequence of SEQ ID NOs: 349, 350 and 351, respectively; and (ii) a VL comprising a VL CDR1, a VL CDR2, and a VL CDR3 having an amino acid sequence of SEQ ID NOs: 352, 353 and 354, respectively; (D) a VH comprising a VH CDR1, a VH CDR2, and a VH CDR3 having an amino acid sequence of SEQ ID NOs: 355, 356 and 357, respectively; and (ii) a VL comprising a VL CDR1, a VL CDR2, and a VL CDR3 having an amino acid sequence of SEQ ID NOs: 358, 359 and 360, respectively; or (E) a VH comprising a VH CDR1, a VH CDR2, and a VH CDR3 having an amino acid sequence of SEQ ID NOs: 361, 362 and 363, respectively; and (ii) a VL comprising a VL CDR1, a VL CDR2, and a VL CDR3 having an amino acid sequence of SEQ ID NOs: 364, 365 and 366, respectively; (XI.) (A) a VH comprising a VH CDR1, a VH CDR2, and a VH CDR3 having an amino acid sequence of SEQ ID NOs: 371, 372 and 373, respectively; and (ii) a VL comprising a VL CDR1, a VL CDR2, and a VL CDR3 having an amino acid sequence of SEQ ID NOs: 374, 375 and 376, respectively; (B) a VH comprising a VH CDR1, a VH CDR2, and a VH CDR3 having an amino acid sequence of SEQ ID NOs: 377, 378 and 379, respectively; and (ii) a VL comprising a VL CDR1, a VL CDR2, and a VL CDR3 having an amino acid sequence of SEQ ID NOs: 380, 381 and 382, respectively; (C) a VH comprising a VH CDR1, a VH CDR2, and a VH CDR3 having an amino acid sequence of SEQ ID NOs: 383, 384 and 385, respectively; and (ii) a VL comprising a VL CDR1, a VL CDR2, and a VL CDR3 having an amino acid sequence of SEQ ID NOs: 386, 387 and 388, respectively; (D) a VH comprising a VH CDR1, a VH CDR2, and a VH CDR3 having an amino acid sequence of SEQ ID NOs: 389, 390 and 391, respectively; and (ii) a VL comprising a VL CDR1, a VL CDR2, and a VL CDR3 having an amino acid sequence of SEQ ID NOs: 392, 393 and 394, respectively; or (E) a VH comprising a VH CDR1, a VH CDR2, and a VH CDR3 having an amino acid sequence of SEQ ID NOs: 395, 396 and 397, respectively; and (ii) a VL comprising a VL CDR1, a VL CDR2, and a VL CDR3 having an amino acid sequence of SEQ ID NOs: 398, 399 and 400, respectively; or (XII.) (A) a VH comprising a VH CDR1, a VH CDR2, and a VH CDR3 having an amino acid sequence of SEQ ID NOs: 405, 406 and 407, respectively; and (ii) a VL comprising a VL CDR1, a VL CDR2, and a VL CDR3 having an amino acid sequence of SEQ ID NOs: 408, 409 and 410, respectively; (B) a VH comprising a VH CDR1, a VH CDR2, and a VH CDR3 having an amino acid sequence of SEQ ID NOs: 411, 412 and 413, respectively; and (ii) a VL comprising a VL CDR1, a VL CDR2, and a VL CDR3 having an amino acid sequence of SEQ ID NOs: 414, 415 and 416, respectively; (C) a VH comprising a VH CDR1, a VH CDR2, and a VH CDR3 having an amino acid sequence of SEQ ID NOs: 417, 418 and 419, respectively; and (ii) a VL comprising a VL CDR1, a VL CDR2, and a VL CDR3 having an amino acid sequence of SEQ ID NOs: 420, 421 and 422, respectively; (D) a VH comprising a VH CDR1, a VH CDR2, and a VH CDR3 having an amino acid sequence of SEQ ID NOs: 423, 424 and 425, respectively; and (ii) a VL comprising a VL CDR1, a VL CDR2, and a VL CDR3 having an amino acid sequence of SEQ ID NOs: 426, 427 and 428, respectively; or (E) a VH comprising a VH CDR1, a VH CDR2, and a VH CDR3 having an amino acid sequence of SEQ ID NOs: 429, 430 and 431, respectively; and (ii) a VL comprising a VL CDR1, a VL CDR2, and a VL CDR3 having an amino acid sequence of SEQ ID NOs: 432, 433 and 434, respectively.

Embodiment 97 provides the antibody of any one of embodiments 94 to 96, comprising: (a) (i) a VH having an amino acid sequence having at least 95% identity to an amino acid sequence of SEQ ID NO: 61; and (ii) a VL having an amino acid sequence having at least 95% identity to an amino acid sequence of SEQ ID NO:62; (b) (i) a VH having an amino acid sequence having at least 95% identity to an amino acid sequence of SEQ ID NO:95; and (ii) a VL having an amino acid sequence having at least 95% identity to an amino acid sequence of SEQ ID NO:96; (c) (i) a VH having an amino acid sequence having at least 95% identity to an amino acid sequence of SEQ ID NO: 129; and (ii) a VL having an amino acid sequence having at least 95% identity to an amino acid sequence of SEQ ID NO: 130; (d) (i) a VH having an amino acid sequence having at least 95% identity to an amino acid sequence of SEQ ID NO: 163; and (ii) a VL having an amino acid sequence having at least 95% identity to an amino acid sequence of SEQ ID NO: 164; (e) (i) a VH having an amino acid sequence having at least 95% identity to an amino acid sequence of SEQ ID NO:197; and (ii) a VL having an amino acid sequence having at least 95% identity to an amino acid sequence of SEQ ID NO:198; (f) (i) a VH having an amino acid sequence having at least 95% identity to an amino acid sequence of SEQ ID NO:231; and (ii) a VL having an amino acid sequence having at least 95% identity to an amino acid sequence of SEQ ID NO:232; (g) (i) a VH having an amino acid sequence having at least 95% identity to an amino acid sequence of SEQ ID NO: 265; and (ii) a VL having an amino acid sequence having at least 95% identity to an amino acid sequence of SEQ ID NO:266; (h) (i) a VH having an amino acid sequence having at least 95% identity to an amino acid sequence of SEQ ID NO:299; and (ii) a VL having an amino acid sequence having at least 95% identity to an amino acid sequence of SEQ ID NO:300; (i) (i) a VH having an amino acid sequence having at least 95% identity to an amino acid sequence of SEQ ID NO:333; and (ii) a VL having an amino acid sequence having at least 95% identity to an amino acid sequence of SEQ ID NO:334; (j) (i) a VH having an amino acid sequence having at least 95% identity to an amino acid sequence of SEQ ID NO: 367; and (ii) a VL having an amino acid sequence having at least 95% identity to an amino acid sequence of SEQ ID NO:368; (k) (i) a VH having an amino acid sequence having at least 95% identity to an amino acid sequence of SEQ ID NO:401; and (ii) a VL having an amino acid sequence having at least 95% identity to an amino acid sequence of SEQ ID NO:402; or (l) (i) a VH having an amino acid sequence having at least 95% identity to an amino acid sequence of SEQ ID NO:435; and (ii) a VL having an amino acid sequence having at least 95% identity to an amino acid sequence of SEQ ID NO: 436.

Embodiment 98 provides the antibody of embodiment 97, comprising: (a) (i) a VH having an amino acid sequence SEQ ID NO:61; and (ii) a VL having an amino acid sequence SEQ ID NO:62; (b) (i) a VH having an amino acid sequence SEQ ID NO:95; and (ii) a VL having an amino acid sequence SEQ ID NO: 96; (c) (i) a VH having an amino acid sequence SEQ ID NO:129; and (ii) a VL having an amino acid sequence SEQ ID NO: 130; (d) (i) a VH having an amino acid sequence SEQ ID NO:163; and (ii) a VL having an amino acid sequence SEQ ID NO: 164; (e) (i) a VH having an amino acid sequence SEQ ID NO: 197; and (ii) a VL having an amino acid sequence SEQ ID NO:198; (f) (i) a VH having an amino acid sequence SEQ ID NO:231; and (ii) a VL having an amino acid sequence SEQ ID NO:232; (g) (i) a VH having an amino acid sequence SEQ ID NO:265; and (ii) a VL having an amino acid sequence SEQ ID NO: 266; (h) (i) a VH having an amino acid sequence SEQ ID NO:299; and (ii) a VL having an amino acid sequence SEQ ID NO:300; (i) (i) a VH having an amino acid sequence SEQ ID NO:333; and (ii) a VL having an amino acid sequence SEQ ID NO:334; (j) (i) a VH having an amino acid sequence SEQ ID NO: 367; and (ii) a VL having an amino acid sequence SEQ ID NO:368; (k) (i) a VH having an amino acid sequence SEQ ID NO:401; and (ii) a VL having an amino acid sequence SEQ ID NO:402; or (l) (i) a VH having an amino acid sequence SEQ ID NO:435; and (ii) a VL having an amino acid sequence SEQ ID NO: 436.

Embodiment 99 provides the antibody of any one of embodiments 94 to 97, comprising: (a) (i) a heavy chain having an amino acid sequence having at least 95% identity to an amino acid sequence of SEQ ID NO:63; and (ii) a light chain having an amino acid sequence having at least 95% identity to an amino acid sequence of SEQ ID NO:64; (b) (i) a heavy chain having an amino acid sequence having at least 95% identity to an amino acid sequence of SEQ ID NO:97; and (ii) a light chain having an amino acid sequence having at least 95% identity to an amino acid sequence of SEQ ID NO:98; (c) (i) a heavy chain having an amino acid sequence having at least 95% identity to an amino acid sequence of SEQ ID NO: 131; and (ii) a light chain having an amino acid sequence having at least 95% identity to an amino acid sequence of SEQ ID NO:132; (d) (i) a heavy chain having an amino acid sequence having at least 95% identity to an amino acid sequence of SEQ ID NO:165; and (ii) a light chain having an amino acid sequence having at least 95% identity to an amino acid sequence of SEQ ID NO:166; (e) (i) a heavy chain having an amino acid sequence having at least 95% identity to an amino acid sequence of SEQ ID NO: 199; and (ii) a light chain having an amino acid sequence having at least 95% identity to an amino acid sequence of SEQ ID NO:200; (f) (i) a heavy chain having an amino acid sequence having at least 95% identity to an amino acid sequence of SEQ ID NO:233; and (ii) a light chain having an amino acid sequence having at least 95% identity to an amino acid sequence of SEQ ID NO:234; (g) (i) a heavy chain having an amino acid sequence having at least 95% identity to an amino acid sequence of SEQ ID NO: 267; and (ii) a light chain having an amino acid sequence having at least 95% identity to an amino acid sequence of SEQ ID NO:268; (h) (i) a heavy chain having an amino acid sequence having at least 95% identity to an amino acid sequence of SEQ ID NO:301; and (ii) a light chain having an amino acid sequence having at least 95% identity to an amino acid sequence of SEQ ID NO:302; (i) (i) a heavy chain having an amino acid sequence having at least 95% identity to an amino acid sequence of SEQ ID NO: 335; and (ii) a light chain having an amino acid sequence having at least 95% identity to an amino acid sequence of SEQ ID NO:336; (j) (i) a heavy chain having an amino acid sequence having at least 95% identity to an amino acid sequence of SEQ ID NO:369; and (ii) a light chain having an amino acid sequence having at least 95% identity to an amino acid sequence of SEQ ID NO:370; (k) (i) a heavy chain having an amino acid sequence having at least 95% identity to an amino acid sequence of SEQ ID NO: 403; and (ii) a light chain having an amino acid sequence having at least 95% identity to an amino acid sequence of SEQ ID NO:404; or (l) (i) a heavy chain having an amino acid sequence having at least 95% identity to an amino acid sequence of SEQ ID NO:437; and (ii) a light chain having an amino acid sequence having at least 95% identity to an amino acid sequence of SEQ ID NO:438.

Embodiment 100 provides the antibody of embodiment 99, comprising: (a) (i) a heavy chain having an amino acid sequence SEQ ID NO:63; and (ii) a light chain having an amino acid sequence SEQ ID NO: 64; (b) (i) a heavy chain having an amino acid sequence SEQ ID NO:97; and (ii) a light chain having an amino acid sequence SEQ ID NO:98; (c) (i) a heavy chain having an amino acid sequence SEQ ID NO: 131; and (ii) a light chain having an amino acid sequence SEQ ID NO:132; (d) (i) a heavy chain having an amino acid sequence SEQ ID NO:165; and (ii) a light chain having an amino acid sequence SEQ ID NO:166; (e) (i) a heavy chain having an amino acid sequence SEQ ID NO:199; and (ii) a light chain having an amino acid sequence SEQ ID NO:200; (f) (i) a heavy chain having an amino acid sequence SEQ ID NO:233; and (ii) a light chain having an amino acid sequence SEQ ID NO:234; (g) (i) a heavy chain having an amino acid sequence SEQ ID NO:267; and (ii) a light chain having an amino acid sequence SEQ ID NO:268; (h) (i) a heavy chain having an amino acid sequence SEQ ID NO:301; and (ii) a light chain having an amino acid sequence SEQ ID NO:302; (i) (i) a heavy chain having an amino acid sequence SEQ ID NO:335; and (ii) a light chain having an amino acid sequence SEQ ID NO:336; (j) (i) a heavy chain having an amino acid sequence SEQ ID NO:369; and (ii) a light chain having an amino acid sequence SEQ ID NO:370; (k) (i) a heavy chain having an amino acid sequence SEQ ID NO:403; and (ii) a light chain having an amino acid sequence SEQ ID NO:404; or (l) (i) a heavy chain having an amino acid sequence SEQ ID NO:437; and (ii) a light chain having an amino acid sequence SEQ ID NO:438.

Embodiment 101 provides the antibody of embodiment 94 to 98, wherein the antibody is a humanized antibody or a fully human antibody.

Embodiment 102 provides the antibody of any one of embodiments 94 to 101, wherein the antibody is an IgG antibody.

Embodiment 103 provides the antibody of embodiment 102, wherein the IgG antibody is an IgG1, IgG2, IgG3, or IgG4 antibody.

Embodiment 104 provides the antibody of any one of embodiments 94 to 98, 102 or 103, wherein the antibody comprises a kappa light chain.

Embodiment 105 provides the antibody of any one of embodiments 94 to 98, 102 or 103, wherein the antibody comprises a lambda light chain.

Embodiment 106 provides the antibody of any one of embodiments 94 to 105, wherein the antibody is a monoclonal antibody.

Embodiment 107 provides the antibody of any one of embodiments 94 to 106, wherein the antibody specifically binds to Sdc2.

Embodiment 108 provides the antibody of any one of embodiments 94 to 107, wherein the Sdc2 is present on the surface of an endothelial cell.

Embodiment 109 provides the antibody of any one of embodiments 94 to 107, wherein the Sdc2 is present on the surface of a neural cell.

Embodiment 110 provides the antibody of any one of embodiments 94 to 109, wherein the antibody is multivalent.

Embodiment 111 provides the antibody of any one of embodiments 94 to 110, wherein the antibody is a multispecific antibody.

Embodiment 112 provides a nucleic acid encoding the antibody of any one of embodiments 94 to 111.

Embodiment 113 provides a vector comprising the nucleic acid of embodiment 112.

Embodiment 114 provides a host cell comprising the vector of embodiment 113.

Embodiment 115 provides a kit comprising the vector of embodiment 113 and packaging for the same.

Embodiment 116 provides a kit comprising the antibody of any one of embodiments 94 to 111 and packaging for the same.

Embodiment 117 provides a pharmaceutical composition comprising the antibody of any one of embodiments 94 to 111, and a pharmaceutically acceptable carrier.

Embodiment 118 provides a method of producing the pharmaceutical composition of embodiment 117, comprising combining the antibody with a pharmaceutically acceptable carrier to obtain the pharmaceutical composition.

Embodiment 119 provides a method of reducing vascular cell permeability, comprising contacting the vascular cells with the antibody of any one of embodiments 94 to 111.

Embodiment 120 provides a method of reducing endothelial cell permeability, comprising contacting the endothelial cells with the antibody of any one of embodiments 94 to 111.

Embodiment 121 provides a method of reducing VEGFA-induced endothelial cell permeability, comprising contacting the endothelial cells with the antibody of any one of embodiments 94 to 111, either before, during or after the endothelial cells are contacted with the VEGFA.

Embodiment 122 provides a method of reducing vascular permeability in a subject, comprising administering to the subject an effective amount of the antibody of any one of embodiments 94 to 111.

Embodiment 123 provides a method of reducing vascular leakage in a subject, comprising administering to the subject an effective amount of the antibody of any one of embodiments 94 to 111.

Embodiment 124 provides a method of reducing endothelial permeability in a subject, comprising administering to the subject an effective amount of the antibody of any one of embodiments 94 to 111.

Embodiment 125 provides the method of any one of embodiments 122 to 124, wherein the subject has a disease caused all or in part by cells expressing Sdc2.

Embodiment 126 provides a method of preventing, treating, or modulating a disease caused all or in part by cells expressing Sdc2, comprising administering to the subject an effective amount of the antibody of any one of embodiments 94 to 111.

Embodiment 127 provides the method of embodiment 126, wherein the cells are endothelial cells.

Embodiment 128 provides the method of embodiment 122 to 127, wherein the disease is associated with vascular permeability or vascular leakage.

Embodiment 129 provides the method of any one of embodiments 122 to 128, wherein the disease is an ARDS, a COVID-19-induced ARDS, a hemorrhagic stroke, an ischemic stroke, a neurological disease in which the BBB is altered or disrupted, Parkinson's Disease, Alzheimer's disease, Huntington's Disease, a peripheral neuropathy, a traumatic brain injury, epilepsy, multiple sclerosis, a neovascular eye disease, a cardiovascular disease, a myocardial infarction, congestive heart failure, a blunt trauma injury, a peripheral vascular disease, a lymphedema, POEMS Syndrome, a pediatric capillary leak syndrome, an adult capillary leak syndrome, a hydrocephalus, a lymphedema, an inflammation-associated edema, an inflammatory disease, systemic lupus erythematosus; a rheumatoid arthritis cardiovascular disease, a neovascular eye disease, AMD, diabetic retinopathy, a stroke, an ischemic stroke, a hemorrhagic stroke, or a cancer.

Embodiment 130 provides the method of any one of embodiments 122 to 129, wherein the subject is a human.

Embodiment 131 provides a method for treating ischemic stroke in a subject, wherein the method comprises administering to the subject an antibody that binds Sdc2 or an antigen binding fragment thereof, wherein the antibody or antigen binding fragment comprises: (a) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:61; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:62; (b) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 95; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:96; (c) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 129; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 130; (d) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 163; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 164; (e) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 197; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 198; (f) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:231; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:232; (g) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:265; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:266; (h) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:299; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:300; (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 333; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:334; (j) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:367; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:368; (k) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:401; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:402; or (l) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:435; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 436.

Embodiment 132 provides the method of embodiment 131, wherein (i) the VH CDR1, VH CDR2, VH CDR3, VL CDR1, VL CDR2, and VL CDR3 amino acid sequences are according to the Kabat numbering system; (ii) the VH CDR1, VH CDR2, VH CDR3, VL CDR1, VL CDR2, and VL CDR3 amino acid sequences are according to the Chothia numbering system; (iii) the VH CDR1, VH CDR2, VH CDR3, VL CDR1, VL CDR2, and VL CDR3 amino acid sequences are according to the AbM numbering system; (iv) the VH CDR1, VH CDR2, VH CDR3, VL CDR1, VL CDR2, and VL CDR3 amino acid sequences are according to the Contact numbering system; or (v) the VH CDR1, VH CDR2, VH CDR3, VL CDR1, VL CDR2, and VL CDR3 amino acid sequences are according to the IMGT numbering system.

Embodiment 133 provides the method of embodiment 131 or 132, wherein the antibody that binds Sdc2 or the antigen binding fragment thereof comprises: (I) (A) a VH comprising a VH CDR1, a VH CDR2, and a VH CDR3 having an amino acid sequence of SEQ ID NOs: 31, 32 and 33, respectively; and (ii) a VL comprising a VL CDR1, a VL CDR2, and a VL CDR3 having an amino acid sequence of SEQ ID NOs: 34, 35 and 36, respectively; (B) a VH comprising a VH CDR1, a VH CDR2, and a VH CDR3 having an amino acid sequence of SEQ ID NOs: 37, 38 and 39, respectively; and (ii) a VL comprising a VL CDR1, a VL CDR2, and a VL CDR3 having an amino acid sequence of SEQ ID NOs: 40, 41 and 42, respectively; (C) a VH comprising a VH CDR1, a VH CDR2, and a VH CDR3 having an amino acid sequence of SEQ ID NOs: 43, 44 and 45, respectively; and (ii) a VL comprising a VL CDR1, a VL CDR2, and a VL CDR3 having an amino acid sequence of SEQ ID NOs: 46, 47 and 48, respectively; (D) a VH comprising a VH CDR1, a VH CDR2, and a VH CDR3 having an amino acid sequence of SEQ ID NOs: 49, 50 and 51, respectively; and (ii) a VL comprising a VL CDR1, a VL CDR2, and a VL CDR3 having an amino acid sequence of SEQ ID NOs: 52, 53 and 54, respectively; or (E) a VH comprising a VH CDR1, a VH CDR2, and a VH CDR3 having an amino acid sequence of SEQ ID NOs: 55, 56 and 57, respectively; and (ii) a VL comprising a VL CDR1, a VL CDR2, and a VL CDR3 having an amino acid sequence of SEQ ID NOs: 58, 59 and 60, respectively; (II) (A) a VH comprising a VH CDR1, a VH CDR2, and a VH CDR3 having an amino acid sequence of SEQ ID NOs: 65, 66 and 67, respectively; and (ii) a VL comprising a VL CDR1, a VL CDR2, and a VL CDR3 having an amino acid sequence of SEQ ID NOs: 68, 89 and 70, respectively; (B) a VH comprising a VH CDR1, a VH CDR2, and a VH CDR3 having an amino acid sequence of SEQ ID NOs: 71, 72 and 73, respectively; and (ii) a VL comprising a VL CDR1, a VL CDR2, and a VL CDR3 having an amino acid sequence of SEQ ID NOs: 74, 75 and 76, respectively; (C) a VH comprising a VH CDR1, a VH CDR2, and a VH CDR3 having an amino acid sequence of SEQ ID NOs: 77, 78 and 79, respectively; and (ii) a VL comprising a VL CDR1, a VL CDR2, and a VL CDR3 having an amino acid sequence of SEQ ID NOs: 80, 81 and 82, respectively; (D) a VH comprising a VH CDR1, a VH CDR2, and a VH CDR3 having an amino acid sequence of SEQ ID NOs: 83, 84 and 85, respectively; and (ii) a VL comprising a VL CDR1, a VL CDR2, and a VL CDR3 having an amino acid sequence of SEQ ID NOs: 86, 87 and 88, respectively; or (E) a VH comprising a VH CDR1, a VH CDR2, and a VH CDR3 having an amino acid sequence of SEQ ID NOs: 89, 90 and 91, respectively; and (ii) a VL comprising a VL CDR1, a VL CDR2, and a VL CDR3 having an amino acid sequence of SEQ ID NOs: 92, 93 and 94, respectively; (III.) (A) a VH comprising a VH CDR1, a VH CDR2, and a VH CDR3 having an amino acid sequence of SEQ ID NOs: 99, 100 and 101, respectively; and (ii) a VL comprising a VL CDR1, a VL CDR2, and a VL CDR3 having an amino acid sequence of SEQ ID NOs: 102,103 and 104, respectively; (B) a VH comprising a VH CDR1, a VH CDR2, and a VH CDR3 having an amino acid sequence of SEQ ID NOs: 105, 106 and 107, respectively; and (ii) a VL comprising a VL CDR1, a VL CDR2, and a VL CDR3 having an amino acid sequence of SEQ ID NOs: 108, 109 and 110, respectively; (C) a VH comprising a VH CDR1, a VH CDR2, and a VH CDR3 having an amino acid sequence of SEQ ID NOs: 111, 112 and 113, respectively; and (ii) a VL comprising a VL CDR1, a VL CDR2, and a VL CDR3 having an amino acid sequence of SEQ ID NOs: 114, 115 and 116, respectively; (D) a VH comprising a VH CDR1, a VH CDR2, and a VH CDR3 having an amino acid sequence of SEQ ID NOs: 117, 118 and 119, respectively; and (ii) a VL comprising a VL CDR1, a VL CDR2, and a VL CDR3 having an amino acid sequence of SEQ ID NOs: 120, 121 and 122, respectively; or (E) a VH comprising a VH CDR1, a VH CDR2, and a VH CDR3 having an amino acid sequence of SEQ ID NOs: 123, 124 and 125, respectively; and (ii) a VL comprising a VL CDR1, a VL CDR2, and a VL CDR3 having an amino acid sequence of SEQ ID NOs: 126, 127 and 128, respectively; (IV.) (A) a VH comprising a VH CDR1, a VH CDR2, and a VH CDR3 having an amino acid sequence of SEQ ID NOs: 133, 134 and 135, respectively; and (ii) a VL comprising a VL CDR1, a VL CDR2, and a VL CDR3 having an amino acid sequence of SEQ ID NOs: 136, 137 and 138, respectively; (B) a VH comprising a VH CDR1, a VH CDR2, and a VH CDR3 having an amino acid sequence of SEQ ID NOs: 139, 140 and 141, respectively; and (ii) a VL comprising a VL CDR1, a VL CDR2, and a VL CDR3 having an amino acid sequence of SEQ ID NOs: 142, 143 and 144, respectively; (C) a VH comprising a VH CDR1, a VH CDR2, and a VH CDR3 having an amino acid sequence of SEQ ID NOs: 145, 146 and 147, respectively; and (ii) a VL comprising a VL CDR1, a VL CDR2, and a VL CDR3 having an amino acid sequence of SEQ ID NOs: 148, 149 and 150, respectively; (D) a VH comprising a VH CDR1, a VH CDR2, and a VH CDR3 having an amino acid sequence of SEQ ID NOs: 151, 152 and 153, respectively; and (ii) a VL comprising a VL CDR1, a VL CDR2, and a VL CDR3 having an amino acid sequence of SEQ ID NOs: 154, 155 and 156, respectively; or (E) a VH comprising a VH CDR1, a VH CDR2, and a VH CDR3 having an amino acid sequence of SEQ ID NOs: 157, 158 and 159, respectively; and (ii) a VL comprising a VL CDR1, a VL CDR2, and a VL CDR3 having an amino acid sequence of SEQ ID NOs: 160, 161 and 162, respectively; (V.) (A) a VH comprising a VH CDR1, a VH CDR2, and a VH CDR3 having an amino acid sequence of SEQ ID NOs: 167, 168 and 169, respectively; and (ii) a VL comprising a VL CDR1, a VL CDR2, and a VL CDR3 having an amino acid sequence of SEQ ID NOs: 170, 171 and 172, respectively; (B) a VH comprising a VH CDR1, a VH CDR2, and a VH CDR3 having an amino acid sequence of SEQ ID NOs: 173, 174 and 175, respectively; and (ii) a VL comprising a VL CDR1, a VL CDR2, and a VL CDR3 having an amino acid sequence of SEQ ID NOs: 176, 177 and 178, respectively; (C) a VH comprising a VH CDR1, a VH CDR2, and a VH CDR3 having an amino acid sequence of SEQ ID NOs: 179, 180 and 181, respectively; and (ii) a VL comprising a VL CDR1, a VL CDR2, and a VL CDR3 having an amino acid sequence of SEQ ID NOs: 182, 183 and 184, respectively; (D) a VH comprising a VH CDR1, a VH CDR2, and a VH CDR3 having an amino acid sequence of SEQ ID NOs: 185, 186 and 187, respectively; and (ii) a VL comprising a VL CDR1, a VL CDR2, and a VL CDR3 having an amino acid sequence of SEQ ID NOs: 188, 189 and 190, respectively; or (E) a VH comprising a VH CDR1, a VH CDR2, and a VH CDR3 having an amino acid sequence of SEQ ID NOs: 191, 192 and 193, respectively; and (ii) a VL comprising a VL CDR1, a VL CDR2, and a VL CDR3 having an amino acid sequence of SEQ ID NOs: 194, 195 and 196, respectively; (VI.) (A) a VH comprising a VH CDR1, a VH CDR2, and a VH CDR3 having an amino acid sequence of SEQ ID NOs: 201, 202 and 203, respectively; and (ii) a VL comprising a VL CDR1, a VL CDR2, and a VL CDR3 having an amino acid sequence of SEQ ID NOs: 204, 205 and 206, respectively; (B) a VH comprising a VH CDR1, a VH CDR2, and a VH CDR3 having an amino acid sequence of SEQ ID NOs: 207, 208 and 209, respectively; and (ii) a VL comprising a VL CDR1, a VL CDR2, and a VL CDR3 having an amino acid sequence of SEQ ID NOs: 210, 211 and 212, respectively; (C) a VH comprising a VH CDR1, a VH CDR2, and a VH CDR3 having an amino acid sequence of SEQ ID NOs: 213, 214 and 215, respectively; and (ii) a VL comprising a VL CDR1, a VL CDR2, and a VL CDR3 having an amino acid sequence of SEQ ID NOs: 216, 217 and 218, respectively; (D) a VH comprising a VH CDR1, a VH CDR2, and a VH CDR3 having an amino acid sequence of SEQ ID NOs: 219, 220 and 221, respectively; and (ii) a VL comprising a VL CDR1, a VL CDR2, and a VL CDR3 having an amino acid sequence of SEQ ID NOs: 222, 223 and 224, respectively; or (E) a VH comprising a VH CDR1, a VH CDR2, and a VH CDR3 having an amino acid sequence of SEQ ID NOs: 225, 226 and 227, respectively; and (ii) a VL comprising a VL CDR1, a VL CDR2, and a VL CDR3 having an amino acid sequence of SEQ ID NOs: 228, 229 and 230, respectively; (VII.) (A) a VH comprising a VH CDR1, a VH CDR2, and a VH CDR3 having an amino acid sequence of SEQ ID NOs: 235, 236 and 237, respectively; and (ii) a VL comprising a VL CDR1, a VL CDR2, and a VL CDR3 having an amino acid sequence of SEQ ID NOs: 238, 239 and 240, respectively; (B) a VH comprising a VH CDR1, a VH CDR2, and a VH CDR3 having an amino acid sequence of SEQ ID NOs: 241, 242 and 243, respectively; and (ii) a VL comprising a VL CDR1, a VL CDR2, and a VL CDR3 having an amino acid sequence of SEQ ID NOs: 244, 245 and 246, respectively; (C) a VH comprising a VH CDR1, a VH CDR2, and a VH CDR3 having an amino acid sequence of SEQ ID NOs: 247, 248 and 249, respectively; and (ii) a VL comprising a VL CDR1, a VL CDR2, and a VL CDR3 having an amino acid sequence of SEQ ID NOs: 250, 251 and 252, respectively; (D) a VH comprising a VH CDR1, a VH CDR2, and a VH CDR3 having an amino acid sequence of SEQ ID NOs: 253, 254 and 255, respectively; and (ii) a VL comprising a VL CDR1, a VL CDR2, and a VL CDR3 having an amino acid sequence of SEQ ID NOs: 256, 257 and 258, respectively; or (E) a VH comprising a VH CDR1, a VH CDR2, and a VH CDR3 having an amino acid sequence of SEQ ID NOs: 259, 260 and 261, respectively; and (ii) a VL comprising a VL CDR1, a VL CDR2, and a VL CDR3 having an amino acid sequence of SEQ ID NOs: 262, 263 and 264, respectively; (VIII.) (A) a VH comprising a VH CDR1, a VH CDR2, and a VH CDR3 having an amino acid sequence of SEQ ID NOs: 269, 270 and 271, respectively; and (ii) a VL comprising a VL CDR1, a VL CDR2, and a VL CDR3 having an amino acid sequence of SEQ ID NOs: 272, 273 and 274, respectively; (B) a VH comprising a VH CDR1, a VH CDR2, and a VH CDR3 having an amino acid sequence of SEQ ID NOs: 275, 276 and 277, respectively; and (ii) a VL comprising a VL CDR1, a VL CDR2, and a VL CDR3 having an amino acid sequence of SEQ ID NOs: 278, 279 and 280, respectively; (C) a VH comprising a VH CDR1, a VH CDR2, and a VH CDR3 having an amino acid sequence of SEQ ID NOs: 281, 282 and 283, respectively; and (ii) a VL comprising a VL CDR1, a VL CDR2, and a VL CDR3 having an amino acid sequence of SEQ ID NOs: 284, 285 and 286, respectively; (D) a VH comprising a VH CDR1, a VH CDR2, and a VH CDR3 having an amino acid sequence of SEQ ID NOs: 287, 288 and 289, respectively; and (ii) a VL comprising a VL CDR1, a VL CDR2, and a VL CDR3 having an amino acid sequence of SEQ ID NOs: 290, 292 and 292, respectively; or (E) a VH comprising a VH CDR1, a VH CDR2, and a VH CDR3 having an amino acid sequence of SEQ ID NOs: 293, 294 and 295, respectively; and (ii) a VL comprising a VL CDR1, a VL CDR2, and a VL CDR3 having an amino acid sequence of SEQ ID NOs: 296, 297 and 298, respectively; (IX). (A) a VH comprising a VH CDR1, a VH CDR2, and a VH CDR3 having an amino acid sequence of SEQ ID NOs: 303, 304 and 305, respectively; and (ii) a VL comprising a VL CDR1, a VL CDR2, and a VL CDR3 having an amino acid sequence of SEQ ID NOs: 306, 307 and 308, respectively; (B) a VH comprising a VH CDR1, a VH CDR2, and a VH CDR3 having an amino acid sequence of SEQ ID NOs: 309, 310 and 311, respectively; and (ii) a VL comprising a VL CDR1, a VL CDR2, and a VL CDR3 having an amino acid sequence of SEQ ID NOs: 312, 313 and 314, respectively; (C) a VH comprising a VH CDR1, a VH CDR2, and a VH CDR3 having an amino acid sequence of SEQ ID NOs: 315, 316 and 317, respectively; and (ii) a VL comprising a VL CDR1, a VL CDR2, and a VL CDR3 having an amino acid sequence of SEQ ID NOs: 318, 319 and 320, respectively; (D) a VH comprising a VH CDR1, a VH CDR2, and a VH CDR3 having an amino acid sequence of SEQ ID NOs: 321, 322 and 323, respectively; and (ii) a VL comprising a VL CDR1, a VL CDR2, and a VL CDR3 having an amino acid sequence of SEQ ID NOs: 324, 325 and 326, respectively; or (E) a VH comprising a VH CDR1, a VH CDR2, and a VH CDR3 having an amino acid sequence of SEQ ID NOs: 327, 328 and 329, respectively; and (ii) a VL comprising a VL CDR1, a VL CDR2, and a VL CDR3 having an amino acid sequence of SEQ ID NOs: 330, 331 and 332, respectively; (X.) (A) a VH comprising a VH CDR1, a VH CDR2, and a VH CDR3 having an amino acid sequence of SEQ ID NOs: 337, 338 and 339, respectively; and (ii) a VL comprising a VL CDR1, a VL CDR2, and a VL CDR3 having an amino acid sequence of SEQ ID NOs: 340, 341 and 342, respectively; (B) a VH comprising a VH CDR1, a VH CDR2, and a VH CDR3 having an amino acid sequence of SEQ ID NOs: 343, 344 and 345, respectively; and (ii) a VL comprising a VL CDR1, a VL CDR2, and a VL CDR3 having an amino acid sequence of SEQ ID NOs: 346, 347 and 348, respectively; (C) a VH comprising a VH CDR1, a VH CDR2, and a VH CDR3 having an amino acid sequence of SEQ ID NOs: 349, 350 and 351, respectively; and (ii) a VL comprising a VL CDR1, a VL CDR2, and a VL CDR3 having an amino acid sequence of SEQ ID NOs: 352, 353 and 354, respectively; (D) a VH comprising a VH CDR1, a VH CDR2, and a VH CDR3 having an amino acid sequence of SEQ ID NOs: 355, 356 and 357, respectively; and (ii) a VL comprising a VL CDR1, a VL CDR2, and a VL CDR3 having an amino acid sequence of SEQ ID NOs: 358, 359 and 360, respectively; or (E) a VH comprising a VH CDR1, a VH CDR2, and a VH CDR3 having an amino acid sequence of SEQ ID NOs: 361, 362 and 363, respectively; and (ii) a VL comprising a VL CDR1, a VL CDR2, and a VL CDR3 having an amino acid sequence of SEQ ID NOs: 364, 365 and 366, respectively; (XI.) (A) a VH comprising a VH CDR1, a VH CDR2, and a VH CDR3 having an amino acid sequence of SEQ ID NOs: 371, 372 and 373, respectively; and (ii) a VL comprising a VL CDR1, a VL CDR2, and a VL CDR3 having an amino acid sequence of SEQ ID NOs: 374, 375 and 376, respectively; (B) a VH comprising a VH CDR1, a VH CDR2, and a VH CDR3 having an amino acid sequence of SEQ ID NOs: 377, 378 and 379, respectively; and (ii) a VL comprising a VL CDR1, a VL CDR2, and a VL CDR3 having an amino acid sequence of SEQ ID NOs: 380, 381 and 382, respectively; (C) a VH comprising a VH CDR1, a VH CDR2, and a VH CDR3 having an amino acid sequence of SEQ ID NOs: 383, 384 and 385, respectively; and (ii) a VL comprising a VL CDR1, a VL CDR2, and a VL CDR3 having an amino acid sequence of SEQ ID NOs: 386, 387 and 388, respectively; (D) a VH comprising a VH CDR1, a VH CDR2, and a VH CDR3 having an amino acid sequence of SEQ ID NOs: 389, 390 and 391, respectively; and (ii) a VL comprising a VL CDR1, a VL CDR2, and a VL CDR3 having an amino acid sequence of SEQ ID NOs: 392, 393 and 394, respectively; or (E) a VH comprising a VH CDR1, a VH CDR2, and a VH CDR3 having an amino acid sequence of SEQ ID NOs: 395, 396 and 397, respectively; and (ii) a VL comprising a VL CDR1, a VL CDR2, and a VL CDR3 having an amino acid sequence of SEQ ID NOs: 398, 399 and 400, respectively; or (XII.) (A) a VH comprising a VH CDR1, a VH CDR2, and a VH CDR3 having an amino acid sequence of SEQ ID NOs: 405, 406 and 407, respectively; and (ii) a VL comprising a VL CDR1, a VL CDR2, and a VL CDR3 having an amino acid sequence of SEQ ID NOs: 408, 409 and 410, respectively; (B) a VH comprising a VH CDR1, a VH CDR2, and a VH CDR3 having an amino acid sequence of SEQ ID NOs: 411, 412 and 413, respectively; and (ii) a VL comprising a VL CDR1, a VL CDR2, and a VL CDR3 having an amino acid sequence of SEQ ID NOs: 414, 415 and 416, respectively; (C) a VH comprising a VH CDR1, a VH CDR2, and a VH CDR3 having an amino acid sequence of SEQ ID NOs: 417, 418 and 419, respectively; and (ii) a VL comprising a VL CDR1, a VL CDR2, and a VL CDR3 having an amino acid sequence of SEQ ID NOs: 420, 421 and 422, respectively; (D) a VH comprising a VH CDR1, a VH CDR2, and a VH CDR3 having an amino acid sequence of SEQ ID NOs: 423, 424 and 425, respectively; and (ii) a VL comprising a VL CDR1, a VL CDR2, and a VL CDR3 having an amino acid sequence of SEQ ID NOs: 426, 427 and 428, respectively; or (E) a VH comprising a VH CDR1, a VH CDR2, and a VH CDR3 having an amino acid sequence of SEQ ID NOs: 429, 430 and 431, respectively; and (ii) a VL comprising a VL CDR1, a VL CDR2, and a VL CDR3 having an amino acid sequence of SEQ ID NOs: 432, 433 and 434, respectively.

Embodiment 134 provides the method of any one of embodiments 131 to 133, wherein the antibody that binds Sdc2 or the antigen binding fragment thereof comprises: (a) (i) a VH having an amino acid sequence having at least 95% identity to an amino acid sequence of SEQ ID NO:61; and (ii) a VL having an amino acid sequence having at least 95% identity to an amino acid sequence of SEQ ID NO:62; (b) (i) a VH having an amino acid sequence having at least 95% identity to an amino acid sequence of SEQ ID NO:95; and (ii) a VL having an amino acid sequence having at least 95% identity to an amino acid sequence of SEQ ID NO:96; (c) (i) a VH having an amino acid sequence having at least 95% identity to an amino acid sequence of SEQ ID NO:129; and (ii) a VL having an amino acid sequence having at least 95% identity to an amino acid sequence of SEQ ID NO: 130; (d) (i) a VH having an amino acid sequence having at least 95% identity to an amino acid sequence of SEQ ID NO: 163; and (ii) a VL having an amino acid sequence having at least 95% identity to an amino acid sequence of SEQ ID NO: 164; (e) (i) a VH having an amino acid sequence having at least 95% identity to an amino acid sequence of SEQ ID NO: 197; and (ii) a VL having an amino acid sequence having at least 95% identity to an amino acid sequence of SEQ ID NO: 198; (f) (i) a VH having an amino acid sequence having at least 95% identity to an amino acid sequence of SEQ ID NO:231; and (ii) a VL having an amino acid sequence having at least 95% identity to an amino acid sequence of SEQ ID NO:232; (g) (i) a VH having an amino acid sequence having at least 95% identity to an amino acid sequence of SEQ ID NO:265; and (ii) a VL having an amino acid sequence having at least 95% identity to an amino acid sequence of SEQ ID NO: 266; (h) (i) a VH having an amino acid sequence having at least 95% identity to an amino acid sequence of SEQ ID NO:299; and (ii) a VL having an amino acid sequence having at least 95% identity to an amino acid sequence of SEQ ID NO:300; (i) (i) a VH having an amino acid sequence having at least 95% identity to an amino acid sequence of SEQ ID NO:333; and (ii) a VL having an amino acid sequence having at least 95% identity to an amino acid sequence of SEQ ID NO:334; (j) (i) a VH having an amino acid sequence having at least 95% identity to an amino acid sequence of SEQ ID NO:367; and (ii) a VL having an amino acid sequence having at least 95% identity to an amino acid sequence of SEQ ID NO: 368; (k) (i) a VH having an amino acid sequence having at least 95% identity to an amino acid sequence of SEQ ID NO:401; and (ii) a VL having an amino acid sequence having at least 95% identity to an amino acid sequence of SEQ ID NO:402; or (l) (i) a VH having an amino acid sequence having at least 95% identity to an amino acid sequence of SEQ ID NO:435; and (ii) a VL having an amino acid sequence having at least 95% identity to an amino acid sequence of SEQ ID NO:436.

Embodiment 135 provides the method of embodiment 134, wherein the antibody that binds Sdc2 or the antigen binding fragment thereof comprises: (a) (i) a VH having an amino acid sequence SEQ ID NO: 61; and (ii) a VL having an amino acid sequence SEQ ID NO:62; (b) (i) a VH having an amino acid sequence SEQ ID NO:95; and (ii) a VL having an amino acid sequence SEQ ID NO:96; (c) (i) a VH having an amino acid sequence SEQ ID NO: 129; and (ii) a VL having an amino acid sequence SEQ ID NO: 130; (d) (i) a VH having an amino acid sequence SEQ ID NO:163; and (ii) a VL having an amino acid sequence SEQ ID NO: 164; (e) (i) a VH having an amino acid sequence SEQ ID NO:197; and (ii) a VL having an amino acid sequence SEQ ID NO: 198; (f) (i) a VH having an amino acid sequence SEQ ID NO: 231; and (ii) a VL having an amino acid sequence SEQ ID NO:232; (g) (i) a VH having an amino acid sequence SEQ ID NO:265; and (ii) a VL having an amino acid sequence SEQ ID NO:266; (h) (i) a VH having an amino acid sequence SEQ ID NO:299; and (ii) a VL having an amino acid sequence SEQ ID NO: 300; (i) (i) a VH having an amino acid sequence SEQ ID NO:333; and (ii) a VL having an amino acid sequence SEQ ID NO:334; (j) (i) a VH having an amino acid sequence SEQ ID NO:367; and (ii) a VL having an amino acid sequence SEQ ID NO:368; (k) (i) a VH having an amino acid sequence SEQ ID NO: 401; and (ii) a VL having an amino acid sequence SEQ ID NO:402; or (l) (i) a VH having an amino acid sequence SEQ ID NO:435; and (ii) a VL having an amino acid sequence SEQ ID NO:436.

Embodiment 136 provides the method of any one of embodiments 131 to 134, wherein the antibody that binds Sdc2 or the antigen binding fragment thereof comprises: (a) (i) a heavy chain having an amino acid sequence having at least 95% identity to an amino acid sequence of SEQ ID NO:63; and (ii) a light chain having an amino acid sequence having at least 95% identity to an amino acid sequence of SEQ ID NO:64; (b) (i) a heavy chain having an amino acid sequence having at least 95% identity to an amino acid sequence of SEQ ID NO:97; and (ii) a light chain having an amino acid sequence having at least 95% identity to an amino acid sequence of SEQ ID NO:98; (c) (i) a heavy chain having an amino acid sequence having at least 95% identity to an amino acid sequence of SEQ ID NO:131; and (ii) a light chain having an amino acid sequence having at least 95% identity to an amino acid sequence of SEQ ID NO: 132; (d) (i) a heavy chain having an amino acid sequence having at least 95% identity to an amino acid sequence of SEQ ID NO:165; and (ii) a light chain having an amino acid sequence having at least 95% identity to an amino acid sequence of SEQ ID NO:166; (e) (i) a heavy chain having an amino acid sequence having at least 95% identity to an amino acid sequence of SEQ ID NO: 199; and (ii) a light chain having an amino acid sequence having at least 95% identity to an amino acid sequence of SEQ ID NO: 200; (f) (i) a heavy chain having an amino acid sequence having at least 95% identity to an amino acid sequence of SEQ ID NO:233; and (ii) a light chain having an amino acid sequence having at least 95% identity to an amino acid sequence of SEQ ID NO:234; (g) (i) a heavy chain having an amino acid sequence having at least 95% identity to an amino acid sequence of SEQ ID NO:267; and (ii) a light chain having an amino acid sequence having at least 95% identity to an amino acid sequence of SEQ ID NO: 268; (h) (i) a heavy chain having an amino acid sequence having at least 95% identity to an amino acid sequence of SEQ ID NO:301; and (ii) a light chain having an amino acid sequence having at least 95% identity to an amino acid sequence of SEQ ID NO:302; (i) (i) a heavy chain having an amino acid sequence having at least 95% identity to an amino acid sequence of SEQ ID NO:335; and (ii) a light chain having an amino acid sequence having at least 95% identity to an amino acid sequence of SEQ ID NO: 336; (j) (i) a heavy chain having an amino acid sequence having at least 95% identity to an amino acid sequence of SEQ ID NO:369; and (ii) a light chain having an amino acid sequence having at least 95% identity to an amino acid sequence of SEQ ID NO:370; (k) (i) a heavy chain having an amino acid sequence having at least 95% identity to an amino acid sequence of SEQ ID NO:403; and (ii) a light chain having an amino acid sequence having at least 95% identity to an amino acid sequence of SEQ ID NO: 404; or (l) (i) a heavy chain having an amino acid sequence having at least 95% identity to an amino acid sequence of SEQ ID NO:437; and (ii) a light chain having an amino acid sequence having at least 95% identity to an amino acid sequence of SEQ ID NO:438.

Embodiment 137 provides the method of embodiment 136, wherein the antibody that binds Sdc2 or the antigen binding fragment thereof comprises: (a) (i) a heavy chain having an amino acid sequence SEQ ID NO:63; and (ii) a light chain having an amino acid sequence SEQ ID NO:64; (b) (i) a heavy chain having an amino acid sequence SEQ ID NO:97; and (ii) a light chain having an amino acid sequence SEQ ID NO:98; (c) (i) a heavy chain having an amino acid sequence SEQ ID NO:131; and (ii) a light chain having an amino acid sequence SEQ ID NO:132; (d) (i) a heavy chain having an amino acid sequence SEQ ID NO: 165; and (ii) a light chain having an amino acid sequence SEQ ID NO:166; (e) (i) a heavy chain having an amino acid sequence SEQ ID NO: 199; and (ii) a light chain having an amino acid sequence SEQ ID NO:200; (f) (i) a heavy chain having an amino acid sequence SEQ ID NO:233; and (ii) a light chain having an amino acid sequence SEQ ID NO:234; (g) (i) a heavy chain having an amino acid sequence SEQ ID NO:267; and (ii) a light chain having an amino acid sequence SEQ ID NO:268; (h) (i) a heavy chain having an amino acid sequence SEQ ID NO:301; and (ii) a light chain having an amino acid sequence SEQ ID NO:302; (i) (i) a heavy chain having an amino acid sequence SEQ ID NO:335; and (ii) a light chain having an amino acid sequence SEQ ID NO:336; (j) (i) a heavy chain having an amino acid sequence SEQ ID NO:369; and (ii) a light chain having an amino acid sequence SEQ ID NO:370; (k) (i) a heavy chain having an amino acid sequence SEQ ID NO:403; and (ii) a light chain having an amino acid sequence SEQ ID NO:404; or (l) (i) a heavy chain having an amino acid sequence SEQ ID NO:437; and (ii) a light chain having an amino acid sequence SEQ ID NO:438.

Embodiment 138 provides the method of any one of embodiments 131 to 135, wherein the antibody is a humanized antibody or a fully human antibody.

Embodiment 139 provides the method of any one of embodiments 131 to 138, wherein the antibody is an IgG antibody.

Embodiment 140 provides the method of embodiment 139, wherein the IgG antibody is an IgG1, IgG2, IgG3, or IgG4 antibody.

Embodiment 141 provides the method of any one of embodiments 131 to 135, 139 or 140, wherein the antibody comprises a kappa light chain.

Embodiment 142 provides the method of any one of embodiments 131 to 135, 139 or 140, wherein the antibody comprises a lambda light chain.

Embodiment 143 provides the method of any one of embodiments 131 to 142, wherein the antibody is a monoclonal antibody.

Embodiment 144 provides the method of any one of embodiments 131 to 143, wherein the antibody specifically binds to Sdc2.

Embodiment 145 provides the method of any one of embodiments 131 to 144, wherein the Sdc2 is present on the surface of an endothelial cell.

Embodiment 146 provides the method of any one of embodiments 131 to 144, wherein the Sdc2 is present on the surface of a neural cell.

Embodiment 147 provides the method of any one of embodiments 131 to 146, wherein the antibody is multivalent.

Embodiment 148 provides the method of any one of embodiments 131 to 147, wherein the antibody is a multispecific antibody.

Embodiment 149 provides the method of any one of embodiments 131 to 148, wherein the subject is a human suffering from or at risk of developing ischemic stroke.

Embodiment 150 provides the method of any one of embodiments 131 to 149, wherein the subject has a brain lesion area associated with the ischemic stroke.

Embodiment 151 provides the method of embodiment 150, wherein upon the administering endothelial cell permeability in or surrounding the brain lesion area of the subject is reduced; optionally wherein the endothelial cell permeability is reduced at least about 10%, at least about 20%, at least about 30%, at least about 40%, at least about 50%, at least about 60%, at least about 70%, at least about 80%, at least about 90%, or at least about 95%.

Embodiment 152 provides the method of embodiment 151, wherein the endothelial cell permeability is VEGFA-induced endothelial cell permeability.

Embodiment 153 provides the method of embodiment 151, wherein at least some of the endothelial cells expresses Sdc2.

Embodiment 154 provides the method of embodiment 150, wherein upon the administering the vascular permeability or vascular leakage in or surrounding the brain lesion area of the subject is reduced; optionally wherein the vascular permeability is reduced at least about 10%, at least about 20%, at least about 30%, at least about 40%, at least about 50%, at least about 60%, at least about 70%, at least about 80%, at least about 90%, or at least about 95%.

Embodiment 155 provides the method of embodiment 150, wherein the brain lesion area comprises a penumbra, and wherein upon the administering, the penumbra is reduced; optionally wherein the penumbra area is reduced at least about 10%, at least about 20%, at least about 30%, at least about 40%, at least about 50%, at least about 60%, at least about 70%, at least about 80%, at least about 90%, or at least about 95% in size.

Embodiment 156 provides the method of embodiment 150 or 555, wherein the brain lesion area comprises an edema, and wherein upon the administering, the edema is reduced; optionally wherein the edema area is reduced at least about 10%, at least about 20%, at least about 30%, at least about 40%, at least about 50%, at least about 60%, at least about 70%, at least about 80%, at least about 90%, or at least about 95% in size.

Embodiment 157 provides the method of embodiment 150, 155, or 156, wherein the brain lesion area comprises an infarct, and wherein upon the administering, the infarct is reduced; optionally wherein the stoke area is reduced at least about 10%, at least about 20%, at least about 30%, at least about 40%, at least about 50%, at least about 60%, at least about 70%, at least about 80%, at least about 90%, or at least about 95% in size.

Embodiment 158 provides the method of any one of embodiments 155 to 157, wherein the reduction in size is measured in area or volume of the brain lesion.

Embodiment 159 provides the method of any one of embodiments 155 to 158, wherein the reduction in size is detected by MRI.

In another set of embodiments, provided are the following:

Embodiment B1 provides a method for preventing, managing, and treating AMD in a subject in need thereof, wherein the method comprises administering to the subject a therapeutic effective amount of an antibody that binds Sdc2 or an antigen binding fragment thereof.

Embodiment B2 provides the method of embodiment B1, wherein the anti-Sdc2 antibody or antigen binding fragment thereof binds to a region in the extracellular domain of Sdc2 that correspond to amino acids 123 to 140 of human Sdc2 (SEQ ID NO:25).

Embodiment B3 provides the method of embodiment B1 or B2, wherein the anti-Sdc2 antibody or antigen binding fragment thereof inhibits binding of Dep-1 to Sdc2.

Embodiment B4 provides the method of any one of embodiments B1 to B3, wherein upon administering of the antibody or antigen binding fragment thereof, endothelial permeability in an eye fundus tissue of the subject is reduced; optionally wherein the endothelial permeability is reduced at least about 10%, at least about 20%, at least about 30%, at least about 40%, at least about 50%, at least about 60%, at least about 70%, at least about 80%, at least about 90%, or at least about 95%.

Embodiment B5 provides the method of embodiment B4, wherein the endothelial permeability is VEGFA-induced endothelial permeability.

Embodiment B6 provides the method of embodiment B4 or B5, wherein the endothelial permeability is measured by fundus fluorescein angiography (FFA).

Embodiment B7 provides the method of embodiment B6, wherein at least some of the endothelial cells expresses Sdc2.

Embodiment B8 provides the method of any one of embodiments B1 to B3, wherein upon administering of the antibody or antigen binding fragment thereof, the vascular permeability in an eye fundus tissue the subject is reduced; optionally wherein the vascular permeability is reduced at least about 10%, at least about 20%, at least about 30%, at least about 40%, at least about 50%, at least about 60%, at least about 70%, at least about 80%, at least about 90%, or at least about 95%.

Embodiment B9 provides the method of embodiment B8, wherein the vascular permeability is measured by FFA.

Embodiment B10 provides the method of any one of embodiments B1 to B3, wherein upon administering of the antibody or antigen binding fragment thereof, Dep-1 expression on surface of cells in an eye fundus tissue is upregulated.

Embodiment B11 provides the method of any one of embodiments B1 to B3, wherein upon administering of the antibody or antigen binding fragment thereof, dephosphorylation of vascular endothelial growth factor receptor 2 (VEGFR2) protein at residue Y951 is enhanced in cells in an eye fundus tissue.

Embodiment B12 provides the method of any one of embodiments B1 to B3, wherein upon administering of the antibody or antigen binding fragment thereof, inflammation in an eye fundus tissue is reduced.

Embodiment B13 provides the method of embodiment B12, wherein expression of one or more inflammatory marker in the eye fundus tissue is reduced; optionally wherein the inflammatory marker is selected from pro-inflammatory cytokines and immune cell surface proteins; optionally wherein the inflammatory marker is F4/80.

Embodiment B14 provides the method of any one of embodiments B1 to B3, wherein upon administering of the antibody or antigen binding fragment thereof, does not impact angiogenesis in an eye fundus tissue.

Embodiment B15 provides the method of embodiment B14, wherein the angiogenesis is choroidal neovascularization (CNV).

Embodiment B16 provides the method of embodiment B14 or B15, wherein expression of one or more endothelial marker remains substantially the same in the eye fundus tissue; optionally wherein the endothelial marker is ERG; optionally wherein the endothelial marker is CD31.

Embodiment B17 provides the method of any one of embodiments B4 to B16, wherein the eye fundus tissue is selected from retina, macula and choroid of the eye.

Embodiment B18 provides the method of any one of embodiments BI to B3, wherein upon administering of the antibody or antigen binding fragment thereof, the central retinal thickness of an eye of the subject is reduced; optionally wherein the central retinal thickness is reduced at least about 10%, at least about 20%, at least about 30%, at least about 40%, at least about 50%, at least about 60%, at least about 70%, at least about 80%, at least about 90%, or at least about 95%.

Embodiment B19 provides the method of embodiment B18, wherein the reduction in central retinal thickness is measured by optical coherence tomography (OCT).

Embodiment B20 provides a method for reducing eye inflammation in a subject in need thereof, wherein the method comprises administering to the subject a therapeutic effective amount of an antibody that binds Sdc2 or an antigen binding fragment thereof, wherein upon administering of the antibody or antigen binding fragment thereof, expression of one or more inflammation marker is reduced in the choroid of an eye of the subject; optionally wherein the inflammation marker is CD31 or F4/80.

Embodiment B21 provides a method of embodiment B20, wherein upon administering of the antibody or antigen binding fragment thereof, expression of one or more endothelial marker in the choroid of the eye remains substantially the same; optionally wherein the inflammatory marker is ERG.

Embodiment B22 provides the method of any one of embodiments B1 to B21, wherein upon administering of the antibody or antigen binding fragment thereof, the vision acuity of the subject is enhanced.

Embodiment B23 provides the method of any one of embodiments B1 to B22, wherein the subject is a human suffering from or at risk of developing AMD.

Embodiment B24 provides the method of any one of embodiments B1 to B23, wherein the antibody or antigen binding fragment comprises: (a) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:61; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:62; (b) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:95; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:96; (c) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 129; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 130; (d) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 163; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 164; (e) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 197; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 198; (f) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:231; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:232; (g) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 265; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:266; (h) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:299; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:300; (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:333; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:334; (j) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:367; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:368; (k) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 401; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:402; or (l) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:435; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:436.

Embodiment B25 provides the method of embodiment B24, wherein (i) the VH CDR1, VH CDR2, VH CDR3, VL CDR1, VL CDR2, and VL CDR3 amino acid sequences are according to the Kabat numbering system; (ii) the VH CDR1, VH CDR2, VH CDR3, VL CDR1, VL CDR2, and VL CDR3 amino acid sequences are according to the Chothia numbering system; (iii) the VH CDR1, VH CDR2, VH CDR3, VL CDR1, VL CDR2, and VL CDR3 amino acid sequences are according to the AbM numbering system; (iv) the VH CDR1, VH CDR2, VH CDR3, VL CDR1, VL CDR2, and VL CDR3 amino acid sequences are according to the Contact numbering system; or (v) the VH CDR1, VH CDR2, VH CDR3, VL CDR1, VL CDR2, and VL CDR3 amino acid sequences are according to the IMGT numbering system.

Embodiment B26 provides the method of embodiment B24 or B25, wherein the antibody that binds Sdc2 or the antigen binding fragment thereof comprises: (I) (A) a VH comprising a VH CDR1, a VH CDR2, and a VH CDR3 having an amino acid sequence of SEQ ID NOs: 31, 32 and 33, respectively; and (ii) a VL comprising a VL CDR1, a VL CDR2, and a VL CDR3 having an amino acid sequence of SEQ ID NOs: 34, 35 and 36, respectively; (B) a VH comprising a VH CDR1, a VH CDR2, and a VH CDR3 having an amino acid sequence of SEQ ID NOs: 37, 38 and 39, respectively; and (ii) a VL comprising a VL CDR1, a VL CDR2, and a VL CDR3 having an amino acid sequence of SEQ ID NOs: 40, 41 and 42, respectively; (C) a VH comprising a VH CDR1, a VH CDR2, and a VH CDR3 having an amino acid sequence of SEQ ID NOs: 43, 44 and 45, respectively; and (ii) a VL comprising a VL CDR1, a VL CDR2, and a VL CDR3 having an amino acid sequence of SEQ ID NOs: 46, 47 and 48, respectively; (D) a VH comprising a VH CDR1, a VH CDR2, and a VH CDR3 having an amino acid sequence of SEQ ID NOs: 49, 50 and 51, respectively; and (ii) a VL comprising a VL CDR1, a VL CDR2, and a VL CDR3 having an amino acid sequence of SEQ ID NOs: 52, 53 and 54, respectively; or (E) a VH comprising a VH CDR1, a VH CDR2, and a VH CDR3 having an amino acid sequence of SEQ ID NOs: 55, 56 and 57, respectively; and (ii) a VL comprising a VL CDR1, a VL CDR2, and a VL CDR3 having an amino acid sequence of SEQ ID NOs: 58, 59 and 60, respectively; (II) (A) a VH comprising a VH CDR1, a VH CDR2, and a VH CDR3 having an amino acid sequence of SEQ ID NOs: 65, 66 and 67, respectively; and (ii) a VL comprising a VL CDR1, a VL CDR2, and a VL CDR3 having an amino acid sequence of SEQ ID NOs: 68, 89 and 70, respectively; (B) a VH comprising a VH CDR1, a VH CDR2, and a VH CDR3 having an amino acid sequence of SEQ ID NOs: 71, 72 and 73, respectively; and (ii) a VL comprising a VL CDR1, a VL CDR2, and a VL CDR3 having an amino acid sequence of SEQ ID NOs: 74, 75 and 76, respectively; (C) a VH comprising a VH CDR1, a VH CDR2, and a VH CDR3 having an amino acid sequence of SEQ ID NOs: 77, 78 and 79, respectively; and (ii) a VL comprising a VL CDR1, a VL CDR2, and a VL CDR3 having an amino acid sequence of SEQ ID NOs: 80, 81 and 82, respectively; (D) a VH comprising a VH CDR1, a VH CDR2, and a VH CDR3 having an amino acid sequence of SEQ ID NOs: 83, 84 and 85, respectively; and (ii) a VL comprising a VL CDR1, a VL CDR2, and a VL CDR3 having an amino acid sequence of SEQ ID NOs: 86, 87 and 88, respectively; or (E) a VH comprising a VH CDR1, a VH CDR2, and a VH CDR3 having an amino acid sequence of SEQ ID NOs: 89, 90 and 91, respectively; and (ii) a VL comprising a VL CDR1, a VL CDR2, and a VL CDR3 having an amino acid sequence of SEQ ID NOs: 92, 93 and 94, respectively; (III.) (A) a VH comprising a VH CDR1, a VH CDR2, and a VH CDR3 having an amino acid sequence of SEQ ID NOs: 99, 100 and 101, respectively; and (ii) a VL comprising a VL CDR1, a VL CDR2, and a VL CDR3 having an amino acid sequence of SEQ ID NOs: 102,103 and 104, respectively; (B) a VH comprising a VH CDR1, a VH CDR2, and a VH CDR3 having an amino acid sequence of SEQ ID NOs: 105, 106 and 107, respectively; and (ii) a VL comprising a VL CDR1, a VL CDR2, and a VL CDR3 having an amino acid sequence of SEQ ID NOs: 108, 109 and 110, respectively; (C) a VH comprising a VH CDR1, a VH CDR2, and a VH CDR3 having an amino acid sequence of SEQ ID NOs: 111, 112 and 113, respectively; and (ii) a VL comprising a VL CDR1, a VL CDR2, and a VL CDR3 having an amino acid sequence of SEQ ID NOs: 114, 115 and 116, respectively; (D) a VH comprising a VH CDR1, a VH CDR2, and a VH CDR3 having an amino acid sequence of SEQ ID NOs: 117, 118 and 119, respectively; and (ii) a VL comprising a VL CDR1, a VL CDR2, and a VL CDR3 having an amino acid sequence of SEQ ID NOs: 120, 121 and 122, respectively; or (E) a VH comprising a VH CDR1, a VH CDR2, and a VH CDR3 having an amino acid sequence of SEQ ID NOs: 123, 124 and 125, respectively; and (ii) a VL comprising a VL CDR1, a VL CDR2, and a VL CDR3 having an amino acid sequence of SEQ ID NOs: 126, 127 and 128, respectively; (IV.) (A) a VH comprising a VH CDR1, a VH CDR2, and a VH CDR3 having an amino acid sequence of SEQ ID NOs: 133, 134 and 135, respectively; and (ii) a VL comprising a VL CDR1, a VL CDR2, and a VL CDR3 having an amino acid sequence of SEQ ID NOs: 136, 137 and 138, respectively; (B) a VH comprising a VH CDR1, a VH CDR2, and a VH CDR3 having an amino acid sequence of SEQ ID NOs: 139, 140 and 141, respectively; and (ii) a VL comprising a VL CDR1, a VL CDR2, and a VL CDR3 having an amino acid sequence of SEQ ID NOs: 142, 143 and 144, respectively; (C) a VH comprising a VH CDR1, a VH CDR2, and a VH CDR3 having an amino acid sequence of SEQ ID NOs: 145, 146 and 147, respectively; and (ii) a VL comprising a VL CDR1, a VL CDR2, and a VL CDR3 having an amino acid sequence of SEQ ID NOs: 148, 149 and 150, respectively; (D) a VH comprising a VH CDR1, a VH CDR2, and a VH CDR3 having an amino acid sequence of SEQ ID NOs: 151, 152 and 153, respectively; and (ii) a VL comprising a VL CDR1, a VL CDR2, and a VL CDR3 having an amino acid sequence of SEQ ID NOs: 154, 155 and 156, respectively; or (E) a VH comprising a VH CDR1, a VH CDR2, and a VH CDR3 having an amino acid sequence of SEQ ID NOs: 157, 158 and 159, respectively; and (ii) a VL comprising a VL CDR1, a VL CDR2, and a VL CDR3 having an amino acid sequence of SEQ ID NOs: 160, 161 and 162, respectively; (V.) (A) a VH comprising a VH CDR1, a VH CDR2, and a VH CDR3 having an amino acid sequence of SEQ ID NOs: 167, 168 and 169, respectively; and (ii) a VL comprising a VL CDR1, a VL CDR2, and a VL CDR3 having an amino acid sequence of SEQ ID NOs: 170, 171 and 172, respectively; (B) a VH comprising a VH CDR1, a VH CDR2, and a VH CDR3 having an amino acid sequence of SEQ ID NOs: 173, 174 and 175, respectively; and (ii) a VL comprising a VL CDR1, a VL CDR2, and a VL CDR3 having an amino acid sequence of SEQ ID NOs: 176, 177 and 178, respectively; (C) a VH comprising a VH CDR1, a VH CDR2, and a VH CDR3 having an amino acid sequence of SEQ ID NOs: 179, 180 and 181, respectively; and (ii) a VL comprising a VL CDR1, a VL CDR2, and a VL CDR3 having an amino acid sequence of SEQ ID NOs: 182, 183 and 184, respectively; (D) a VH comprising a VH CDR1, a VH CDR2, and a VH CDR3 having an amino acid sequence of SEQ ID NOs: 185, 186 and 187, respectively; and (ii) a VL comprising a VL CDR1, a VL CDR2, and a VL CDR3 having an amino acid sequence of SEQ ID NOs: 188, 189 and 190, respectively; or (E) a VH comprising a VH CDR1, a VH CDR2, and a VH CDR3 having an amino acid sequence of SEQ ID NOs: 191, 192 and 193, respectively; and (ii) a VL comprising a VL CDR1, a VL CDR2, and a VL CDR3 having an amino acid sequence of SEQ ID NOs: 194, 195 and 196, respectively; (VI.) (A) a VH comprising a VH CDR1, a VH CDR2, and a VH CDR3 having an amino acid sequence of SEQ ID NOs: 201, 202 and 203, respectively; and (ii) a VL comprising a VL CDR1, a VL CDR2, and a VL CDR3 having an amino acid sequence of SEQ ID NOs: 204, 205 and 206, respectively; (B) a VH comprising a VH CDR1, a VH CDR2, and a VH CDR3 having an amino acid sequence of SEQ ID NOs: 207, 208 and 209, respectively; and (ii) a VL comprising a VL CDR1, a VL CDR2, and a VL CDR3 having an amino acid sequence of SEQ ID NOs: 210, 211 and 212, respectively; (C) a VH comprising a VH CDR1, a VH CDR2, and a VH CDR3 having an amino acid sequence of SEQ ID NOs: 213, 214 and 215, respectively; and (ii) a VL comprising a VL CDR1, a VL CDR2, and a VL CDR3 having an amino acid sequence of SEQ ID NOs: 216, 217 and 218, respectively; (D) a VH comprising a VH CDR1, a VH CDR2, and a VH CDR3 having an amino acid sequence of SEQ ID NOs: 219, 220 and 221, respectively; and (ii) a VL comprising a VL CDR1, a VL CDR2, and a VL CDR3 having an amino acid sequence of SEQ ID NOs: 222, 223 and 224, respectively; or (E) a VH comprising a VH CDR1, a VH CDR2, and a VH CDR3 having an amino acid sequence of SEQ ID NOs: 225, 226 and 227, respectively; and (ii) a VL comprising a VL CDR1, a VL CDR2, and a VL CDR3 having an amino acid sequence of SEQ ID NOs: 228, 229 and 230, respectively; (VII.) (A) a VH comprising a VH CDR1, a VH CDR2, and a VH CDR3 having an amino acid sequence of SEQ ID NOs: 235, 236 and 237, respectively; and (ii) a VL comprising a VL CDR1, a VL CDR2, and a VL CDR3 having an amino acid sequence of SEQ ID NOs: 238, 239 and 240, respectively; (B) a VH comprising a VH CDR1, a VH CDR2, and a VH CDR3 having an amino acid sequence of SEQ ID NOs: 241, 242 and 243, respectively; and (ii) a VL comprising a VL CDR1, a VL CDR2, and a VL CDR3 having an amino acid sequence of SEQ ID NOs: 244, 245 and 246, respectively; (C) a VH comprising a VH CDR1, a VH CDR2, and a VH CDR3 having an amino acid sequence of SEQ ID NOs: 247, 248 and 249, respectively; and (ii) a VL comprising a VL CDR1, a VL CDR2, and a VL CDR3 having an amino acid sequence of SEQ ID NOs: 250, 251 and 252, respectively; (D) a VH comprising a VH CDR1, a VH CDR2, and a VH CDR3 having an amino acid sequence of SEQ ID NOs: 253, 254 and 255, respectively; and (ii) a VL comprising a VL CDR1, a VL CDR2, and a VL CDR3 having an amino acid sequence of SEQ ID NOs: 256, 257 and 258, respectively; or (E) a VH comprising a VH CDR1, a VH CDR2, and a VH CDR3 having an amino acid sequence of SEQ ID NOs: 259, 260 and 261, respectively; and (ii) a VL comprising a VL CDR1, a VL CDR2, and a VL CDR3 having an amino acid sequence of SEQ ID NOs: 262, 263 and 264, respectively; (VIII.) (A) a VH comprising a VH CDR1, a VH CDR2, and a VH CDR3 having an amino acid sequence of SEQ ID NOs: 269, 270 and 271, respectively; and (ii) a VL comprising a VL CDR1, a VL CDR2, and a VL CDR3 having an amino acid sequence of SEQ ID NOs: 272, 273 and 274, respectively; (B) a VH comprising a VH CDR1, a VH CDR2, and a VH CDR3 having an amino acid sequence of SEQ ID NOs: 275, 276 and 277, respectively; and (ii) a VL comprising a VL CDR1, a VL CDR2, and a VL CDR3 having an amino acid sequence of SEQ ID NOs: 278, 279 and 280, respectively; (C) a VH comprising a VH CDR1, a VH CDR2, and a VH CDR3 having an amino acid sequence of SEQ ID NOs: 281, 282 and 283, respectively; and (ii) a VL comprising a VL CDR1, a VL CDR2, and a VL CDR3 having an amino acid sequence of SEQ ID NOs: 284, 285 and 286, respectively; (D) a VH comprising a VH CDR1, a VH CDR2, and a VH CDR3 having an amino acid sequence of SEQ ID NOs: 287, 288 and 289, respectively; and (ii) a VL comprising a VL CDR1, a VL CDR2, and a VL CDR3 having an amino acid sequence of SEQ ID NOs: 290, 292 and 292, respectively; or (E) a VH comprising a VH CDR1, a VH CDR2, and a VH CDR3 having an amino acid sequence of SEQ ID NOs: 293, 294 and 295, respectively; and (ii) a VL comprising a VL CDR1, a VL CDR2, and a VL CDR3 having an amino acid sequence of SEQ ID NOs: 296, 297 and 298, respectively; (IX). (A) a VH comprising a VH CDR1, a VH CDR2, and a VH CDR3 having an amino acid sequence of SEQ ID NOs: 303, 304 and 305, respectively; and (ii) a VL comprising a VL CDR1, a VL CDR2, and a VL CDR3 having an amino acid sequence of SEQ ID NOs: 306, 307 and 308, respectively; (B) a VH comprising a VH CDR1, a VH CDR2, and a VH CDR3 having an amino acid sequence of SEQ ID NOs: 309, 310 and 311, respectively; and (ii) a VL comprising a VL CDR1, a VL CDR2, and a VL CDR3 having an amino acid sequence of SEQ ID NOs: 312, 313 and 314, respectively; (C) a VH comprising a VH CDR1, a VH CDR2, and a VH CDR3 having an amino acid sequence of SEQ ID NOs: 315, 316 and 317, respectively; and (ii) a VL comprising a VL CDR1, a VL CDR2, and a VL CDR3 having an amino acid sequence of SEQ ID NOs: 318, 319 and 320, respectively; (D) a VH comprising a VH CDR1, a VH CDR2, and a VH CDR3 having an amino acid sequence of SEQ ID NOs: 321, 322 and 323, respectively; and (ii) a VL comprising a VL CDR1, a VL CDR2, and a VL CDR3 having an amino acid sequence of SEQ ID NOs: 324, 325 and 326, respectively; or (E) a VH comprising a VH CDR1, a VH CDR2, and a VH CDR3 having an amino acid sequence of SEQ ID NOs: 327, 328 and 329, respectively; and (ii) a VL comprising a VL CDR1, a VL CDR2, and a VL CDR3 having an amino acid sequence of SEQ ID NOs: 330, 331 and 332, respectively; (X.) (A) a VH comprising a VH CDR1, a VH CDR2, and a VH CDR3 having an amino acid sequence of SEQ ID NOs: 337, 338 and 339, respectively; and (ii) a VL comprising a VL CDR1, a VL CDR2, and a VL CDR3 having an amino acid sequence of SEQ ID NOs: 340, 341 and 342, respectively; (B) a VH comprising a VH CDR1, a VH CDR2, and a VH CDR3 having an amino acid sequence of SEQ ID NOs: 343, 344 and 345, respectively; and (ii) a VL comprising a VL CDR1, a VL CDR2, and a VL CDR3 having an amino acid sequence of SEQ ID NOs: 346, 347 and 348, respectively; (C) a VH comprising a VH CDR1, a VH CDR2, and a VH CDR3 having an amino acid sequence of SEQ ID NOs: 349, 350 and 351, respectively; and (ii) a VL comprising a VL CDR1, a VL CDR2, and a VL CDR3 having an amino acid sequence of SEQ ID NOs: 352, 353 and 354, respectively; (D) a VH comprising a VH CDR1, a VH CDR2, and a VH CDR3 having an amino acid sequence of SEQ ID NOs: 355, 356 and 357, respectively; and (ii) a VL comprising a VL CDR1, a VL CDR2, and a VL CDR3 having an amino acid sequence of SEQ ID NOs: 358, 359 and 360, respectively; or (E) a VH comprising a VH CDR1, a VH CDR2, and a VH CDR3 having an amino acid sequence of SEQ ID NOs: 361, 362 and 363, respectively; and (ii) a VL comprising a VL CDR1, a VL CDR2, and a VL CDR3 having an amino acid sequence of SEQ ID NOs: 364, 365 and 366, respectively; (XI.) (A) a VH comprising a VH CDR1, a VH CDR2, and a VH CDR3 having an amino acid sequence of SEQ ID NOs: 371, 372 and 373, respectively; and (ii) a VL comprising a VL CDR1, a VL CDR2, and a VL CDR3 having an amino acid sequence of SEQ ID NOs: 374, 375 and 376, respectively; (B) a VH comprising a VH CDR1, a VH CDR2, and a VH CDR3 having an amino acid sequence of SEQ ID NOs: 377, 378 and 379, respectively; and (ii) a VL comprising a VL CDR1, a VL CDR2, and a VL CDR3 having an amino acid sequence of SEQ ID NOs: 380, 381 and 382, respectively; (C) a VH comprising a VH CDR1, a VH CDR2, and a VH CDR3 having an amino acid sequence of SEQ ID NOs: 383, 384 and 385, respectively; and (ii) a VL comprising a VL CDR1, a VL CDR2, and a VL CDR3 having an amino acid sequence of SEQ ID NOs: 386, 387 and 388, respectively; (D) a VH comprising a VH CDR1, a VH CDR2, and a VH CDR3 having an amino acid sequence of SEQ ID NOs: 389, 390 and 391, respectively; and (ii) a VL comprising a VL CDR1, a VL CDR2, and a VL CDR3 having an amino acid sequence of SEQ ID NOs: 392, 393 and 394, respectively; or (E) a VH comprising a VH CDR1, a VH CDR2, and a VH CDR3 having an amino acid sequence of SEQ ID NOs: 395, 396 and 397, respectively; and (ii) a VL comprising a VL CDR1, a VL CDR2, and a VL CDR3 having an amino acid sequence of SEQ ID NOs: 398, 399 and 400, respectively; or (XII.) (A) a VH comprising a VH CDR1, a VH CDR2, and a VH CDR3 having an amino acid sequence of SEQ ID NOs: 405, 406 and 407, respectively; and (ii) a VL comprising a VL CDR1, a VL CDR2, and a VL CDR3 having an amino acid sequence of SEQ ID NOs: 408, 409 and 410, respectively; (B) a VH comprising a VH CDR1, a VH CDR2, and a VH CDR3 having an amino acid sequence of SEQ ID NOs: 411, 412 and 413, respectively; and (ii) a VL comprising a VL CDR1, a VL CDR2, and a VL CDR3 having an amino acid sequence of SEQ ID NOs: 414, 415 and 416, respectively; (C) a VH comprising a VH CDR1, a VH CDR2, and a VH CDR3 having an amino acid sequence of SEQ ID NOs: 417, 418 and 419, respectively; and (ii) a VL comprising a VL CDR1, a VL CDR2, and a VL CDR3 having an amino acid sequence of SEQ ID NOs: 420, 421 and 422, respectively; (D) a VH comprising a VH CDR1, a VH CDR2, and a VH CDR3 having an amino acid sequence of SEQ ID NOs: 423, 424 and 425, respectively; and (ii) a VL comprising a VL CDR1, a VL CDR2, and a VL CDR3 having an amino acid sequence of SEQ ID NOs: 426, 427 and 428, respectively; or (E) a VH comprising a VH CDR1, a VH CDR2, and a VH CDR3 having an amino acid sequence of SEQ ID NOs: 429, 430 and 431, respectively; and (ii) a VL comprising a VL CDR1, a VL CDR2, and a VL CDR3 having an amino acid sequence of SEQ ID NOs: 432, 433 and 434, respectively.

Embodiment B27 provides the method of any one of embodiments B24 to B26, wherein the antibody that binds Sdc2 or the antigen binding fragment thereof comprises: (a) (i) a VH having an amino acid sequence having at least 95% identity to an amino acid sequence of SEQ ID NO:61; and (ii) a VL having an amino acid sequence having at least 95% identity to an amino acid sequence of SEQ ID NO:62; (b) (i) a VH having an amino acid sequence having at least 95% identity to an amino acid sequence of SEQ ID NO:95; and (ii) a VL having an amino acid sequence having at least 95% identity to an amino acid sequence of SEQ ID NO:96; (c) (i) a VH having an amino acid sequence having at least 95% identity to an amino acid sequence of SEQ ID NO: 129; and (ii) a VL having an amino acid sequence having at least 95% identity to an amino acid sequence of SEQ ID NO: 130; (d) (i) a VH having an amino acid sequence having at least 95% identity to an amino acid sequence of SEQ ID NO: 163; and (ii) a VL having an amino acid sequence having at least 95% identity to an amino acid sequence of SEQ ID NO: 164; (e) (i) a VH having an amino acid sequence having at least 95% identity to an amino acid sequence of SEQ ID NO: 197; and (ii) a VL having an amino acid sequence having at least 95% identity to an amino acid sequence of SEQ ID NO: 198; (f) (i) a VH having an amino acid sequence having at least 95% identity to an amino acid sequence of SEQ ID NO:231; and (ii) a VL having an amino acid sequence having at least 95% identity to an amino acid sequence of SEQ ID NO:232; (g) (i) a VH having an amino acid sequence having at least 95% identity to an amino acid sequence of SEQ ID NO:265; and (ii) a VL having an amino acid sequence having at least 95% identity to an amino acid sequence of SEQ ID NO: 266; (h) (i) a VH having an amino acid sequence having at least 95% identity to an amino acid sequence of SEQ ID NO:299; and (ii) a VL having an amino acid sequence having at least 95% identity to an amino acid sequence of SEQ ID NO:300; (i) (i) a VH having an amino acid sequence having at least 95% identity to an amino acid sequence of SEQ ID NO:333; and (ii) a VL having an amino acid sequence having at least 95% identity to an amino acid sequence of SEQ ID NO:334; (j) (i) a VH having an amino acid sequence having at least 95% identity to an amino acid sequence of SEQ ID NO:367; and (ii) a VL having an amino acid sequence having at least 95% identity to an amino acid sequence of SEQ ID NO: 368; (k) (i) a VH having an amino acid sequence having at least 95% identity to an amino acid sequence of SEQ ID NO:401; and (ii) a VL having an amino acid sequence having at least 95% identity to an amino acid sequence of SEQ ID NO:402; or (l) (i) a VH having an amino acid sequence having at least 95% identity to an amino acid sequence of SEQ ID NO:435; and (ii) a VL having an amino acid sequence having at least 95% identity to an amino acid sequence of SEQ ID NO:436.

Embodiment B28 provides the method of embodiment B27, wherein the antibody that binds Sdc2 or the antigen binding fragment thereof comprises: (a) (i) a VH having an amino acid sequence SEQ ID NO: 61; and (ii) a VL having an amino acid sequence SEQ ID NO:62; (b) (i) a VH having an amino acid sequence SEQ ID NO:95; and (ii) a VL having an amino acid sequence SEQ ID NO:96; (c) (i) a VH having an amino acid sequence SEQ ID NO:129; and (ii) a VL having an amino acid sequence SEQ ID NO: 130; (d) (i) a VH having an amino acid sequence SEQ ID NO:163; and (ii) a VL having an amino acid sequence SEQ ID NO:164; (e) (i) a VH having an amino acid sequence SEQ ID NO:197; and (ii) a VL having an amino acid sequence SEQ ID NO: 198; (f) (i) a VH having an amino acid sequence SEQ ID NO: 231; and (ii) a VL having an amino acid sequence SEQ ID NO:232; (g) (i) a VH having an amino acid sequence SEQ ID NO:265; and (ii) a VL having an amino acid sequence SEQ ID NO:266; (h) (i) a VH having an amino acid sequence SEQ ID NO:299; and (ii) a VL having an amino acid sequence SEQ ID NO: 300; (i) (i) a VH having an amino acid sequence SEQ ID NO:333; and (ii) a VL having an amino acid sequence SEQ ID NO:334; (j) (i) a VH having an amino acid sequence SEQ ID NO:367; and (ii) a VL having an amino acid sequence SEQ ID NO:368; (k) (i) a VH having an amino acid sequence SEQ ID NO: 401; and (ii) a VL having an amino acid sequence SEQ ID NO:402; or (l) (i) a VH having an amino acid sequence SEQ ID NO:435; and (ii) a VL having an amino acid sequence SEQ ID NO:436.

Embodiment B29 provides the method of any one of embodiments B24 to B28, wherein the antibody that binds Sdc2 or the antigen binding fragment thereof comprises: (a) (i) a heavy chain having an amino acid sequence having at least 95% identity to an amino acid sequence of SEQ ID NO:63; and (ii) a light chain having an amino acid sequence having at least 95% identity to an amino acid sequence of SEQ ID NO:64; (b) (i) a heavy chain having an amino acid sequence having at least 95% identity to an amino acid sequence of SEQ ID NO:97; and (ii) a light chain having an amino acid sequence having at least 95% identity to an amino acid sequence of SEQ ID NO:98; (c) (i) a heavy chain having an amino acid sequence having at least 95% identity to an amino acid sequence of SEQ ID NO: 131; and (ii) a light chain having an amino acid sequence having at least 95% identity to an amino acid sequence of SEQ ID NO: 132; (d) (i) a heavy chain having an amino acid sequence having at least 95% identity to an amino acid sequence of SEQ ID NO:165; and (ii) a light chain having an amino acid sequence having at least 95% identity to an amino acid sequence of SEQ ID NO:166; (e) (i) a heavy chain having an amino acid sequence having at least 95% identity to an amino acid sequence of SEQ ID NO: 199; and (ii) a light chain having an amino acid sequence having at least 95% identity to an amino acid sequence of SEQ ID NO: 200; (f) (i) a heavy chain having an amino acid sequence having at least 95% identity to an amino acid sequence of SEQ ID NO:233; and (ii) a light chain having an amino acid sequence having at least 95% identity to an amino acid sequence of SEQ ID NO:234; (g) (i) a heavy chain having an amino acid sequence having at least 95% identity to an amino acid sequence of SEQ ID NO:267; and (ii) a light chain having an amino acid sequence having at least 95% identity to an amino acid sequence of SEQ ID NO: 268; (h) (i) a heavy chain having an amino acid sequence having at least 95% identity to an amino acid sequence of SEQ ID NO:301; and (ii) a light chain having an amino acid sequence having at least 95% identity to an amino acid sequence of SEQ ID NO:302; (i) (i) a heavy chain having an amino acid sequence having at least 95% identity to an amino acid sequence of SEQ ID NO:335; and (ii) a light chain having an amino acid sequence having at least 95% identity to an amino acid sequence of SEQ ID NO: 336; (j) (i) a heavy chain having an amino acid sequence having at least 95% identity to an amino acid sequence of SEQ ID NO:369; and (ii) a light chain having an amino acid sequence having at least 95% identity to an amino acid sequence of SEQ ID NO:370; (k) (i) a heavy chain having an amino acid sequence having at least 95% identity to an amino acid sequence of SEQ ID NO:403; and (ii) a light chain having an amino acid sequence having at least 95% identity to an amino acid sequence of SEQ ID NO: 404; or (l) (i) a heavy chain having an amino acid sequence having at least 95% identity to an amino acid sequence of SEQ ID NO:437; and (ii) a light chain having an amino acid sequence having at least 95% identity to an amino acid sequence of SEQ ID NO:438.

Embodiment B30 provides the method of embodiment B29, wherein the antibody that binds Sdc2 comprises: (a) (i) a heavy chain having an amino acid sequence SEQ ID NO:63; and (ii) a light chain having an amino acid sequence SEQ ID NO:64; (b) (i) a heavy chain having an amino acid sequence SEQ ID NO: 97; and (ii) a light chain having an amino acid sequence SEQ ID NO:98; (c) (i) a heavy chain having an amino acid sequence SEQ ID NO:131; and (ii) a light chain having an amino acid sequence SEQ ID NO:132; (d) (i) a heavy chain having an amino acid sequence SEQ ID NO:165; and (ii) a light chain having an amino acid sequence SEQ ID NO: 166; (e) (i) a heavy chain having an amino acid sequence SEQ ID NO:199; and (ii) a light chain having an amino acid sequence SEQ ID NO:200; (f) (i) a heavy chain having an amino acid sequence SEQ ID NO:233; and (ii) a light chain having an amino acid sequence SEQ ID NO:234; (g) (i) a heavy chain having an amino acid sequence SEQ ID NO:267; and (ii) a light chain having an amino acid sequence SEQ ID NO:268; (h) (i) a heavy chain having an amino acid sequence SEQ ID NO:301; and (ii) a light chain having an amino acid sequence SEQ ID NO:302; (i) (i) a heavy chain having an amino acid sequence SEQ ID NO:335; and (ii) a light chain having an amino acid sequence SEQ ID NO:336; (j) (i) a heavy chain having an amino acid sequence SEQ ID NO:369; and (ii) a light chain having an amino acid sequence SEQ ID NO:370; (k) (i) a heavy chain having an amino acid sequence SEQ ID NO:403; and (ii) a light chain having an amino acid sequence SEQ ID NO:404; or (l) (i) a heavy chain having an amino acid sequence SEQ ID NO:437; and (ii) a light chain having an amino acid sequence SEQ ID NO:438.

Embodiment B31 provides the method of any one of embodiments B24 to B28, wherein the antibody is a humanized antibody or a fully human antibody.

Embodiment B32 provides the method of any one of embodiments B24 to B31, wherein the antibody is an IgG antibody.

Embodiment B33 provides the method of embodiment B32, wherein the IgG antibody is an IgG1, IgG2, IgG3, or IgG4 antibody.

Embodiment B34 provides the method of any one of embodiments B24 to B28, B32 or B33, wherein the antibody comprises a kappa light chain.

Embodiment B35 provides the method of any one of embodiments B24 to B28, B32 or B33, wherein the antibody comprises a lambda light chain.

Embodiment B36 provides the method of any one of embodiments B24 to B35, wherein the antibody is a monoclonal antibody.

In yet another set of embodiments, provides are the following:

Embodiment C1 provides a method for preventing, managing and treating acute myocardial infarction (AMI) in a subject in need thereof, wherein the method comprises administering to the subject a therapeutic effective amount of an antibody that binds Sdc2 or an antigen binding fragment thereof.

Embodiment C2 provides the method of embodiment C1, wherein the anti-Sdc2 antibody or antigen binding fragment thereof binds to a region in the extracellular domain of Sdc2 that correspond to amino acids 123 to 140 of human Sdc2 (SEQ ID NO:25).

Embodiment C3 provides the method of embodiment C1 or C2, wherein the anti-Sdc2 antibody or antigen binding fragment thereof inhibits binding of Dep-1 to Sdc2.

Embodiment C4 provides the method of any one of embodiments C1 to C3, wherein upon administering of the antibody or antigen binding fragment thereof, endothelial permeability in a heart tissue of the subject is reduced; optionally wherein the endothelial permeability is reduced at least about 10%, at least about 20%, at least about 30%, at least about 40%, at least about 50%, at least about 60%, at least about 70%, at least about 80%, at least about 90%, or at least about 95%.

Embodiment C5 provides the method of embodiment C4, wherein the endothelial permeability is VEGFA-induced endothelial permeability.

Embodiment C6 provides the method of embodiment C4 or C5, wherein the endothelial permeability is measured by an Evans Blue assay.

Embodiment C7 provides the method of embodiment C4 or C5, wherein the endothelial permeability is measured by a Dextran perfusion assay.

Embodiment C8 provides the method of embodiment C4, wherein at least some of the endothelial cells expresses Sdc2.

Embodiment C9 provides the method of any one of embodiments C1 to C3, wherein upon administering of the antibody or antigen binding fragment thereof, the vascular permeability in the heart of the subject is reduced; optionally wherein the vascular permeability is reduced at least about 10%, at least about 20%, at least about 30%, at least about 40%, at least about 50%, at least about 60%, at least about 70%, at least about 80%, at least about 90%, or at least about 95%.

Embodiment C10 provides the method of embodiment C9, wherein the vascular permeability is measured by an Evans Blue assay.

Embodiment C11 provides the method of embodiment C9 or C10, wherein the vascular permeability is measured by a Dextran perfusion assay.

Embodiment C12 provides the method of any one of embodiments C1 to C3, wherein upon administering of the antibody or antigen binding fragment thereof, Dep-1 expression on surface of cells in a heart tissue is upregulated.

Embodiment C13 provides the method of any one of embodiments C1 to C3, wherein upon administering of the antibody or antigen binding fragment thereof, dephosphorylation of vascular endothelial growth factor receptor 2 (VEGFR2) protein at residue Y951 is enhanced in cells in a heart tissue.

Embodiment C14 provides the method of any one of embodiments C1 to C3, wherein upon administering of the antibody or antigen binding fragment thereof, inflammation in a heart tissue of the subject is reduced.

Embodiment C15 provides the method of embodiment C14, wherein expression of one or more inflammatory marker in the heart tissue is reduced; optionally wherein the inflammatory marker is selected from pro-inflammatory cytokines and immune cell surface proteins; optionally wherein the inflammatory marker is selected from CD11b, GM-CSF, MIG, CCL11, IL-3, IL-6, and TNF-α.

Embodiment C16 provides the method of embodiment C15, wherein reduction of expression of the one or more inflammatory marker occurred within about 24 hours, within about 36 hours, or within about 72 hours after administration of the antibody or antigen binding fragment thereof.

Embodiment C17 provides the method of any one of embodiments C1 to C3, wherein upon administering of the antibody or antigen binding fragment thereof, the infarct size in the heart of the subject is reduced; optionally wherein the infarct size is reduced at least about 10%, at least about 20%, at least about 30%, at least about 40%, at least about 50%, at least about 60%, at least about 70%, at least about 80%, at least about 90%, or at least about 95%.

Embodiment C18 provides the method of any one of embodiments C1 to C3, wherein upon administering of the antibody or antigen binding fragment thereof, (a) the LVEF of the heart of the subject is enhanced; (b) the cardiac output of the heart of the subject is enhanced; (c) the LV end diastolic diameter (LVEDD) of the heart of the subject is reduced; (d) the LV end systolic diameter (LVESD) of the heart of the subject is reduced; (e) the LV end diastolic volume of the heart of the subject is reduced; (f) the LV end systolic volume of the heart of the subject is reduced; (g) the LV mass of the heart of the subject is reduced; (h) the fractional shortening of the heart of the subject is enhanced; (i) the ejection fraction of the heart of the subject is enhanced; (j) the risk or duration of post-infarct ventricular tachycardia (VT) of the subject is reduced; and/or (k) the risk for the subject to have a heart failure is reduced; optionally where the risk is having the heart failure within 1 month, 2 months, or three months following an episode of AMI.

Embodiment C19 provides the method of embodiment C18, wherein post-infarct VT of the subject is measured by an electrocardiogram with programmed stimulation of the subject's heart; optionally wherein an increase in the number of stimuli for inducing the VT indicates a reduced risk of post-infarct VT in the subject; optionally wherein a reduced duration of induced VT under a hypokalemic condition indicates a reduced risk of post-infarct VT in the subject; optionally wherein a longer cycle length of induced VT indicates a reduced risk of post-infarct VT in the subject.

Embodiment C20 provides the method of any one of embodiments C1 to C19, wherein the subject is a human suffering from or at risk of developing AMI.

Embodiment C21 provides the method of any one of embodiments C1 to C20, wherein the antibody or antigen binding fragment comprises: (a) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:61; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:62; (b) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:95; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:96; (c) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 129; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 130; (d) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 163; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:164; (e) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 197; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 198; (f) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:231; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:232; (g) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 265; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:266; (h) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:299; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:300; (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:333; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:334; (j) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:367; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:368; (k) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO: 401; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:402; or (l) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:435; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:436.

Embodiment C22 provides the method of embodiment C21, wherein (i) the VH CDR1, VH CDR2, VH CDR3, VL CDR1, VL CDR2, and VL CDR3 amino acid sequences are according to the Kabat numbering system; (ii) the VH CDR1, VH CDR2, VH CDR3, VL CDR1, VL CDR2, and VL CDR3 amino acid sequences are according to the Chothia numbering system; (iii) the VH CDR1, VH CDR2, VH CDR3, VL CDR1, VL CDR2, and VL CDR3 amino acid sequences are according to the AbM numbering system; (iv) the VH CDR1, VH CDR2, VH CDR3, VL CDR1, VL CDR2, and VL CDR3 amino acid sequences are according to the Contact numbering system; or (v) the VH CDR1, VH CDR2, VH CDR3, VL CDR1, VL CDR2, and VL CDR3 amino acid sequences are according to the IMGT numbering system.

Embodiment C23 provides the method of embodiment C21 to C22, wherein the antibody that binds Sdc2 or the antigen binding fragment thereof comprises: (I) (A) a VH comprising a VH CDR1, a VH CDR2, and a VH CDR3 having an amino acid sequence of SEQ ID NOs: 31, 32 and 33, respectively; and (ii) a VL comprising a VL CDR1, a VL CDR2, and a VL CDR3 having an amino acid sequence of SEQ ID NOs: 34, 35 and 36, respectively; (B) a VH comprising a VH CDR1, a VH CDR2, and a VH CDR3 having an amino acid sequence of SEQ ID NOs: 37, 38 and 39, respectively; and (ii) a VL comprising a VL CDR1, a VL CDR2, and a VL CDR3 having an amino acid sequence of SEQ ID NOs: 40, 41 and 42, respectively; (C) a VH comprising a VH CDR1, a VH CDR2, and a VH CDR3 having an amino acid sequence of SEQ ID NOs: 43, 44 and 45, respectively; and (ii) a VL comprising a VL CDR1, a VL CDR2, and a VL CDR3 having an amino acid sequence of SEQ ID NOs: 46, 47 and 48, respectively; (D) a VH comprising a VH CDR1, a VH CDR2, and a VH CDR3 having an amino acid sequence of SEQ ID NOs: 49, 50 and 51, respectively; and (ii) a VL comprising a VL CDR1, a VL CDR2, and a VL CDR3 having an amino acid sequence of SEQ ID NOs: 52, 53 and 54, respectively; or (E) a VH comprising a VH CDR1, a VH CDR2, and a VH CDR3 having an amino acid sequence of SEQ ID NOs: 55, 56 and 57, respectively; and (ii) a VL comprising a VL CDR1, a VL CDR2, and a VL CDR3 having an amino acid sequence of SEQ ID NOs: 58, 59 and 60, respectively; (II) (A) a VH comprising a VH CDR1, a VH CDR2, and a VH CDR3 having an amino acid sequence of SEQ ID NOs: 65, 66 and 67, respectively; and (ii) a VL comprising a VL CDR1, a VL CDR2, and a VL CDR3 having an amino acid sequence of SEQ ID NOs: 68, 89 and 70, respectively; (B) a VH comprising a VH CDR1, a VH CDR2, and a VH CDR3 having an amino acid sequence of SEQ ID NOs: 71, 72 and 73, respectively; and (ii) a VL comprising a VL CDR1, a VL CDR2, and a VL CDR3 having an amino acid sequence of SEQ ID NOs: 74, 75 and 76, respectively; (C) a VH comprising a VH CDR1, a VH CDR2, and a VH CDR3 having an amino acid sequence of SEQ ID NOs: 77, 78 and 79, respectively; and (ii) a VL comprising a VL CDR1, a VL CDR2, and a VL CDR3 having an amino acid sequence of SEQ ID NOs: 80, 81 and 82, respectively; (D) a VH comprising a VH CDR1, a VH CDR2, and a VH CDR3 having an amino acid sequence of SEQ ID NOs: 83, 84 and 85, respectively; and (ii) a VL comprising a VL CDR1, a VL CDR2, and a VL CDR3 having an amino acid sequence of SEQ ID NOs: 86, 87 and 88, respectively; or (E) a VH comprising a VH CDR1, a VH CDR2, and a VH CDR3 having an amino acid sequence of SEQ ID NOs: 89, 90 and 91, respectively; and (ii) a VL comprising a VL CDR1, a VL CDR2, and a VL CDR3 having an amino acid sequence of SEQ ID NOs: 92, 93 and 94, respectively; (III.) (A) a VH comprising a VH CDR1, a VH CDR2, and a VH CDR3 having an amino acid sequence of SEQ ID NOs: 99, 100 and 101, respectively; and (ii) a VL comprising a VL CDR1, a VL CDR2, and a VL CDR3 having an amino acid sequence of SEQ ID NOs: 102,103 and 104, respectively; (B) a VH comprising a VH CDR1, a VH CDR2, and a VH CDR3 having an amino acid sequence of SEQ ID NOs: 105, 106 and 107, respectively; and (ii) a VL comprising a VL CDR1, a VL CDR2, and a VL CDR3 having an amino acid sequence of SEQ ID NOs: 108, 109 and 110, respectively; (C) a VH comprising a VH CDR1, a VH CDR2, and a VH CDR3 having an amino acid sequence of SEQ ID NOs: 111, 112 and 113, respectively; and (ii) a VL comprising a VL CDR1, a VL CDR2, and a VL CDR3 having an amino acid sequence of SEQ ID NOs: 114, 115 and 116, respectively; (D) a VH comprising a VH CDR1, a VH CDR2, and a VH CDR3 having an amino acid sequence of SEQ ID NOs: 117, 118 and 119, respectively; and (ii) a VL comprising a VL CDR1, a VL CDR2, and a VL CDR3 having an amino acid sequence of SEQ ID NOs: 120, 121 and 122, respectively; or (E) a VH comprising a VH CDR1, a VH CDR2, and a VH CDR3 having an amino acid sequence of SEQ ID NOs: 123, 124 and 125, respectively; and (ii) a VL comprising a VL CDR1, a VL CDR2, and a VL CDR3 having an amino acid sequence of SEQ ID NOs: 126, 127 and 128, respectively; (IV.) (A) a VH comprising a VH CDR1, a VH CDR2, and a VH CDR3 having an amino acid sequence of SEQ ID NOs: 133, 134 and 135, respectively; and (ii) a VL comprising a VL CDR1, a VL CDR2, and a VL CDR3 having an amino acid sequence of SEQ ID NOs: 136, 137 and 138, respectively; (B) a VH comprising a VH CDR1, a VH CDR2, and a VH CDR3 having an amino acid sequence of SEQ ID NOs: 139, 140 and 141, respectively; and (ii) a VL comprising a VL CDR1, a VL CDR2, and a VL CDR3 having an amino acid sequence of SEQ ID NOs: 142, 143 and 144, respectively; (C) a VH comprising a VH CDR1, a VH CDR2, and a VH CDR3 having an amino acid sequence of SEQ ID NOs: 145, 146 and 147, respectively; and (ii) a VL comprising a VL CDR1, a VL CDR2, and a VL CDR3 having an amino acid sequence of SEQ ID NOs: 148, 149 and 150, respectively; (D) a VH comprising a VH CDR1, a VH CDR2, and a VH CDR3 having an amino acid sequence of SEQ ID NOs: 151, 152 and 153, respectively; and (ii) a VL comprising a VL CDR1, a VL CDR2, and a VL CDR3 having an amino acid sequence of SEQ ID NOs: 154, 155 and 156, respectively; or (E) a VH comprising a VH CDR1, a VH CDR2, and a VH CDR3 having an amino acid sequence of SEQ ID NOs: 157, 158 and 159, respectively; and (ii) a VL comprising a VL CDR1, a VL CDR2, and a VL CDR3 having an amino acid sequence of SEQ ID NOs: 160, 161 and 162, respectively; (V.) (A) a VH comprising a VH CDR1, a VH CDR2, and a VH CDR3 having an amino acid sequence of SEQ ID NOs: 167, 168 and 169, respectively; and (ii) a VL comprising a VL CDR1, a VL CDR2, and a VL CDR3 having an amino acid sequence of SEQ ID NOs: 170, 171 and 172, respectively; (B) a VH comprising a VH CDR1, a VH CDR2, and a VH CDR3 having an amino acid sequence of SEQ ID NOs: 173, 174 and 175, respectively; and (ii) a VL comprising a VL CDR1, a VL CDR2, and a VL CDR3 having an amino acid sequence of SEQ ID NOs: 176, 177 and 178, respectively; (C) a VH comprising a VH CDR1, a VH CDR2, and a VH CDR3 having an amino acid sequence of SEQ ID NOs: 179, 180 and 181, respectively; and (ii) a VL comprising a VL CDR1, a VL CDR2, and a VL CDR3 having an amino acid sequence of SEQ ID NOs: 182, 183 and 184, respectively; (D) a VH comprising a VH CDR1, a VH CDR2, and a VH CDR3 having an amino acid sequence of SEQ ID NOs: 185, 186 and 187, respectively; and (ii) a VL comprising a VL CDR1, a VL CDR2, and a VL CDR3 having an amino acid sequence of SEQ ID NOs: 188, 189 and 190, respectively; or (E) a VH comprising a VH CDR1, a VH CDR2, and a VH CDR3 having an amino acid sequence of SEQ ID NOs: 191, 192 and 193, respectively; and (ii) a VL comprising a VL CDR1, a VL CDR2, and a VL CDR3 having an amino acid sequence of SEQ ID NOs: 194, 195 and 196, respectively; (VI.) (A) a VH comprising a VH CDR1, a VH CDR2, and a VH CDR3 having an amino acid sequence of SEQ ID NOs: 201, 202 and 203, respectively; and (ii) a VL comprising a VL CDR1, a VL CDR2, and a VL CDR3 having an amino acid sequence of SEQ ID NOs: 204, 205 and 206, respectively; (B) a VH comprising a VH CDR1, a VH CDR2, and a VH CDR3 having an amino acid sequence of SEQ ID NOs: 207, 208 and 209, respectively; and (ii) a VL comprising a VL CDR1, a VL CDR2, and a VL CDR3 having an amino acid sequence of SEQ ID NOs: 210, 211 and 212, respectively; (C) a VH comprising a VH CDR1, a VH CDR2, and a VH CDR3 having an amino acid sequence of SEQ ID NOs: 213, 214 and 215, respectively; and (ii) a VL comprising a VL CDR1, a VL CDR2, and a VL CDR3 having an amino acid sequence of SEQ ID NOs: 216, 217 and 218, respectively; (D) a VH comprising a VH CDR1, a VH CDR2, and a VH CDR3 having an amino acid sequence of SEQ ID NOs: 219, 220 and 221, respectively; and (ii) a VL comprising a VL CDR1, a VL CDR2, and a VL CDR3 having an amino acid sequence of SEQ ID NOs: 222, 223 and 224, respectively; or (E) a VH comprising a VH CDR1, a VH CDR2, and a VH CDR3 having an amino acid sequence of SEQ ID NOs: 225, 226 and 227, respectively; and (ii) a VL comprising a VL CDR1, a VL CDR2, and a VL CDR3 having an amino acid sequence of SEQ ID NOs: 228, 229 and 230, respectively; (VII.) (A) a VH comprising a VH CDR1, a VH CDR2, and a VH CDR3 having an amino acid sequence of SEQ ID NOs: 235, 236 and 237, respectively; and (ii) a VL comprising a VL CDR1, a VL CDR2, and a VL CDR3 having an amino acid sequence of SEQ ID NOs: 238, 239 and 240, respectively; (B) a VH comprising a VH CDR1, a VH CDR2, and a VH CDR3 having an amino acid sequence of SEQ ID NOs: 241, 242 and 243, respectively; and (ii) a VL comprising a VL CDR1, a VL CDR2, and a VL CDR3 having an amino acid sequence of SEQ ID NOs: 244, 245 and 246, respectively; (C) a VH comprising a VH CDR1, a VH CDR2, and a VH CDR3 having an amino acid sequence of SEQ ID NOs: 247, 248 and 249, respectively; and (ii) a VL comprising a VL CDR1, a VL CDR2, and a VL CDR3 having an amino acid sequence of SEQ ID NOs: 250, 251 and 252, respectively; (D) a VH comprising a VH CDR1, a VH CDR2, and a VH CDR3 having an amino acid sequence of SEQ ID NOs: 253, 254 and 255, respectively; and (ii) a VL comprising a VL CDR1, a VL CDR2, and a VL CDR3 having an amino acid sequence of SEQ ID NOs: 256, 257 and 258, respectively; or (E) a VH comprising a VH CDR1, a VH CDR2, and a VH CDR3 having an amino acid sequence of SEQ ID NOs: 259, 260 and 261, respectively; and (ii) a VL comprising a VL CDR1, a VL CDR2, and a VL CDR3 having an amino acid sequence of SEQ ID NOs: 262, 263 and 264, respectively; (VIII.) (A) a VH comprising a VH CDR1, a VH CDR2, and a VH CDR3 having an amino acid sequence of SEQ ID NOs: 269, 270 and 271, respectively; and (ii) a VL comprising a VL CDR1, a VL CDR2, and a VL CDR3 having an amino acid sequence of SEQ ID NOs: 272, 273 and 274, respectively; (B) a VH comprising a VH CDR1, a VH CDR2, and a VH CDR3 having an amino acid sequence of SEQ ID NOs: 275, 276 and 277, respectively; and (ii) a VL comprising a VL CDR1, a VL CDR2, and a VL CDR3 having an amino acid sequence of SEQ ID NOs: 278, 279 and 280, respectively; (C) a VH comprising a VH CDR1, a VH CDR2, and a VH CDR3 having an amino acid sequence of SEQ ID NOs: 281, 282 and 283, respectively; and (ii) a VL comprising a VL CDR1, a VL CDR2, and a VL CDR3 having an amino acid sequence of SEQ ID NOs: 284, 285 and 286, respectively; (D) a VH comprising a VH CDR1, a VH CDR2, and a VH CDR3 having an amino acid sequence of SEQ ID NOs: 287, 288 and 289, respectively; and (ii) a VL comprising a VL CDR1, a VL CDR2, and a VL CDR3 having an amino acid sequence of SEQ ID NOs: 290, 292 and 292, respectively; or (E) a VH comprising a VH CDR1, a VH CDR2, and a VH CDR3 having an amino acid sequence of SEQ ID NOs: 293, 294 and 295, respectively; and (ii) a VL comprising a VL CDR1, a VL CDR2, and a VL CDR3 having an amino acid sequence of SEQ ID NOs: 296, 297 and 298, respectively; (IX). (A) a VH comprising a VH CDR1, a VH CDR2, and a VH CDR3 having an amino acid sequence of SEQ ID NOs: 303, 304 and 305, respectively; and (ii) a VL comprising a VL CDR1, a VL CDR2, and a VL CDR3 having an amino acid sequence of SEQ ID NOs: 306, 307 and 308, respectively; (B) a VH comprising a VH CDR1, a VH CDR2, and a VH CDR3 having an amino acid sequence of SEQ ID NOs: 309, 310 and 311, respectively; and (ii) a VL comprising a VL CDR1, a VL CDR2, and a VL CDR3 having an amino acid sequence of CDR3 having an amino acid sequence of SEQ ID NOs: 315, 316 and 317, respectively; and (ii) a VL comprising a VL CDR1, a VL CDR2, and a VL CDR3 having an amino acid sequence of SEQ ID NOs: 318, 319 and 320, respectively; (D) a VH comprising a VH CDR1, a VH CDR2, and a VH CDR3 having an amino acid sequence of SEQ ID NOs: 321, 322 and 323, respectively; and (ii) a VL comprising a VL CDR1, a VL CDR2, and a VL CDR3 having an amino acid sequence of SEQ ID NOs: 324, 325 and 326, respectively; or (E) a VH comprising a VH CDR1, a VH CDR2, and a VH CDR3 having an amino acid sequence of SEQ ID NOs: 327, 328 and 329, respectively; and (ii) a VL comprising a VL CDR1, a VL CDR2, and a VL CDR3 having an amino acid sequence of SEQ ID NOs: 330, 331 and 332, respectively; (X.) (A) a VH comprising a VH CDR1, a VH CDR2, and a VH CDR3 having an amino acid sequence of SEQ ID NOs: 337, 338 and 339, respectively; and (ii) a VL comprising a VL CDR1, a VL CDR2, and a VL CDR3 having an amino acid sequence of SEQ ID NOs: 340, 341 and 342, respectively; (B) a VH comprising a VH CDR1, a VH CDR2, and a VH CDR3 having an amino acid sequence of SEQ ID NOs: 343, 344 and 345, respectively; and (ii) a VL comprising a VL CDR1, a VL CDR2, and a VL CDR3 having an amino acid sequence of SEQ ID NOs: 346, 347 and 348, respectively; (C) a VH comprising a VH CDR1, a VH CDR2, and a VH CDR3 having an amino acid sequence of SEQ ID NOs: 349, 350 and 351, respectively; and (ii) a VL comprising a VL CDR1, a VL CDR2, and a VL CDR3 having an amino acid sequence of SEQ ID NOs: 352, 353 and 354, respectively; (D) a VH comprising a VH CDR1, a VH CDR2, and a VH CDR3 having an amino acid sequence of SEQ ID NOs: 355, 356 and 357, respectively; and (ii) a VL comprising a VL CDR1, a VL CDR2, and a VL CDR3 having an amino acid sequence of SEQ ID NOs: 358, 359 and 360, respectively; or (E) a VH comprising a VH CDR1, a VH CDR2, and a VH CDR3 having an amino acid sequence of SEQ ID NOs: 361, 362 and 363, respectively; and (ii) a VL comprising a VL CDR1, a VL CDR2, and a VL CDR3 having an amino acid sequence of SEQ ID NOs: 364, 365 and 366, respectively; (XI.) (A) a VH comprising a VH CDR1, a VH CDR2, and a VH CDR3 having an amino acid sequence of SEQ ID NOs: 371, 372 and 373, respectively; and (ii) a VL comprising a VL CDR1, a VL CDR2, and a VL CDR3 having an amino acid sequence of SEQ ID NOs: 374, 375 and 376, respectively; (B) a VH comprising a VH CDR1, a VH CDR2, and a VH CDR3 having an amino acid sequence of SEQ ID NOs: 377, 378 and 379, respectively; and (ii) a VL comprising a VL CDR1, a VL CDR2, and a VL CDR3 having an amino acid sequence of SEQ ID NOs: 380, 381 and 382, respectively; (C) a VH comprising a VH CDR1, a VH CDR2, and a VH CDR3 having an amino acid sequence of SEQ ID NOs: 383, 384 and 385, respectively; and (ii) a VL comprising a VL CDR1, a VL CDR2, and a VL CDR3 having an amino acid sequence of SEQ ID NOs: 386, 387 and 388, respectively; (D) a VH comprising a VH CDR1, a VH CDR2, and a VH CDR3 having an amino acid sequence of SEQ ID NOs: 389, 390 and 391, respectively; and (ii) a VL comprising a VL CDR1, a VL CDR2, and a VL CDR3 having an amino acid sequence of SEQ ID NOs: 392, 393 and 394, respectively; or (E) a VH comprising a VH CDR1, a VH CDR2, and a VH CDR3 having an amino acid sequence of SEQ ID NOs: 395, 396 and 397, respectively; and (ii) a VL comprising a VL CDR1, a VL CDR2, and a VL CDR3 having an amino acid sequence of SEQ ID NOs: 398, 399 and 400, respectively; or (XII.) (A) a VH comprising a VH CDR1, a VH CDR2, and a VH CDR3 having an amino acid sequence of SEQ ID NOs: 405, 406 and 407, respectively; and (ii) a VL comprising a VL CDR1, a VL CDR2, and a VL CDR3 having an amino acid sequence of SEQ ID NOs: 408, 409 and 410, respectively; (B) a VH comprising a VH CDR1, a VH CDR2, and a VH CDR3 having an amino acid sequence of SEQ ID NOs: 411, 412 and 413, respectively; and (ii) a VL comprising a VL CDR1, a VL CDR2, and a VL CDR3 having an amino acid sequence of SEQ ID NOs: 414, 415 and 416, respectively; (C) a VH comprising a VH CDR1, a VH CDR2, and a VH CDR3 having an amino acid sequence of SEQ ID NOs: 417, 418 and 419, respectively; and (ii) a VL comprising a VL CDR1, a VL CDR2, and a VL CDR3 having an amino acid sequence of SEQ ID NOs: 420, 421 and 422, respectively; (D) a VH comprising a VH CDR1, a VH CDR2, and a VH CDR3 having an amino acid sequence of SEQ ID NOs: 423, 424 and 425, respectively; and (ii) a VL comprising a VL CDR1, a VL CDR2, and a VL CDR3 having an amino acid sequence of SEQ ID NOs: 426, 427 and 428, respectively; or (E) a VH comprising a VH CDR1, a VH CDR2, and a VH CDR3 having an amino acid sequence of SEQ ID NOs: 429, 430 and 431, respectively; and (ii) a VL comprising a VL CDR1, a VL CDR2, and a VL CDR3 having an amino acid sequence of SEQ ID NOs: 432, 433 and 434, respectively.

Embodiment C24 provides the method of any one of embodiments C21 to C23, wherein the antibody that binds Sdc2 or the antigen binding fragment thereof comprises: (a) (i) a VH having an amino acid sequence having at least 95% identity to an amino acid sequence of SEQ ID NO:61; and (ii) a VL having an amino acid sequence having at least 95% identity to an amino acid sequence of SEQ ID NO:62; (b) (i) a VH having an amino acid sequence having at least 95% identity to an amino acid sequence of SEQ ID NO:95; and (ii) a VL having an amino acid sequence having at least 95% identity to an amino acid sequence of SEQ ID NO:96; (c) (i) a VH having an amino acid sequence having at least 95% identity to an amino acid sequence of SEQ ID NO: 129; and (ii) a VL having an amino acid sequence having at least 95% identity to an amino acid sequence of SEQ ID NO:130; (d) (i) a VH having an amino acid sequence having at least 95% identity to an amino acid sequence of SEQ ID NO: 163; and (ii) a VL having an amino acid sequence having at least 95% identity to an amino acid sequence of SEQ ID NO: 164; (e) (i) a VH having an amino acid sequence having at least 95% identity to an amino acid sequence of SEQ ID NO: 197; and (ii) a VL having an amino acid sequence having at least 95% identity to an amino acid sequence of SEQ ID NO:198; (f) (i) a VH having an amino acid sequence having at least 95% identity to an amino acid sequence of SEQ ID NO:231; and (ii) a VL having an amino acid sequence having at least 95% identity to an amino acid sequence of SEQ ID NO:232; (g) (i) a VH having an amino acid sequence having at least 95% identity to an amino acid sequence of SEQ ID NO:265; and (ii) a VL having an amino acid sequence having at least 95% identity to an amino acid sequence of SEQ ID NO: 266; (h) (i) a VH having an amino acid sequence having at least 95% identity to an amino acid sequence of SEQ ID NO:299; and (ii) a VL having an amino acid sequence having at least 95% identity to an amino acid sequence of SEQ ID NO:300; (i) (i) a VH having an amino acid sequence having at least 95% identity to an amino acid sequence of SEQ ID NO:333; and (ii) a VL having an amino acid sequence having at least 95% identity to an amino acid sequence of SEQ ID NO:334; (j) (i) a VH having an amino acid sequence having at least 95% identity to an amino acid sequence of SEQ ID NO:367; and (ii) a VL having an amino acid sequence having at least 95% identity to an amino acid sequence of SEQ ID NO: 368; (k) (i) a VH having an amino acid sequence having at least 95% identity to an amino acid sequence of SEQ ID NO:401; and (ii) a VL having an amino acid sequence having at least 95% identity to an amino acid sequence of SEQ ID NO:402; or (l) (i) a VH having an amino acid sequence having at least 95% identity to an amino acid sequence of SEQ ID NO:435; and (ii) a VL having an amino acid sequence having at least 95% identity to an amino acid sequence of SEQ ID NO:436.

Embodiment C25 provides the method of embodiment C24, wherein the antibody that binds Sdc2 or the antigen binding fragment thereof comprises: (a) (i) a VH having an amino acid sequence SEQ ID NO: 61; and (ii) a VL having an amino acid sequence SEQ ID NO:62; (b) (i) a VH having an amino acid sequence SEQ ID NO:95; and (ii) a VL having an amino acid sequence SEQ ID NO:96; (c) (i) a VH having an amino acid sequence SEQ ID NO:129; and (ii) a VL having an amino acid sequence SEQ ID NO: 130; (d) (i) a VH having an amino acid sequence SEQ ID NO:163; and (ii) a VL having an amino acid sequence SEQ ID NO: 164; (e) (i) a VH having an amino acid sequence SEQ ID NO:197; and (ii) a VL having an amino acid sequence SEQ ID NO: 198; (f) (i) a VH having an amino acid sequence SEQ ID NO: 231; and (ii) a VL having an amino acid sequence SEQ ID NO:232; (g) (i) a VH having an amino acid sequence SEQ ID NO:265; and (ii) a VL having an amino acid sequence SEQ ID NO:266; (h) (i) a VH having an amino acid sequence SEQ ID NO:299; and (ii) a VL having an amino acid sequence SEQ ID NO: 300; (i) (i) a VH having an amino acid sequence SEQ ID NO:333; and (ii) a VL having an amino acid sequence SEQ ID NO:334; (j) (i) a VH having an amino acid sequence SEQ ID NO:367; and (ii) a VL having an amino acid sequence SEQ ID NO:368; (k) (i) a VH having an amino acid sequence SEQ ID NO: 401; and (ii) a VL having an amino acid sequence SEQ ID NO:402; or (l) (i) a VH having an amino acid sequence SEQ ID NO:435; and (ii) a VL having an amino acid sequence SEQ ID NO:436.

Embodiment C26 provides the method of any one of embodiments C21 to C25, wherein the antibody that binds Sdc2 or the antigen binding fragment thereof comprises: (a) (i) a heavy chain having an amino acid sequence having at least 95% identity to an amino acid sequence of SEQ ID NO:63; and (ii) a light chain having an amino acid sequence having at least 95% identity to an amino acid sequence of SEQ ID NO:64; (b) (i) a heavy chain having an amino acid sequence having at least 95% identity to an amino acid sequence of SEQ ID NO:97; and (ii) a light chain having an amino acid sequence having at least 95% identity to an amino acid sequence of SEQ ID NO:98; (c) (i) a heavy chain having an amino acid sequence having at least 95% identity to an amino acid sequence of SEQ ID NO: 131; and (ii) a light chain having an amino acid sequence having at least 95% identity to an amino acid sequence of SEQ ID NO: 132; (d) (i) a heavy chain having an amino acid sequence having at least 95% identity to an amino acid sequence of SEQ ID NO:165; and (ii) a light chain having an amino acid sequence having at least 95% identity to an amino acid sequence of SEQ ID NO:166; (e) (i) a heavy chain having an amino acid sequence having at least 95% identity to an amino acid sequence of SEQ ID NO: 199; and (ii) a light chain having an amino acid sequence having at least 95% identity to an amino acid sequence of SEQ ID NO: 200; (f) (i) a heavy chain having an amino acid sequence having at least 95% identity to an amino acid sequence of SEQ ID NO:233; and (ii) a light chain having an amino acid sequence having at least 95% identity to an amino acid sequence of SEQ ID NO:234; (g) (i) a heavy chain having an amino acid sequence having at least 95% identity to an amino acid sequence of SEQ ID NO:267; and (ii) a light chain having an amino acid sequence having at least 95% identity to an amino acid sequence of SEQ ID NO: 268; (h) (i) a heavy chain having an amino acid sequence having at least 95% identity to an amino acid sequence of SEQ ID NO:301; and (ii) a light chain having an amino acid sequence having at least 95% identity to an amino acid sequence of SEQ ID NO:302; (i) (i) a heavy chain having an amino acid sequence having at least 95% identity to an amino acid sequence of SEQ ID NO:335; and (ii) a light chain having an amino acid sequence having at least 95% identity to an amino acid sequence of SEQ ID NO: 336; (j) (i) a heavy chain having an amino acid sequence having at least 95% identity to an amino acid sequence of SEQ ID NO:369; and (ii) a light chain having an amino acid sequence having at least 95% identity to an amino acid sequence of SEQ ID NO:370; (k) (i) a heavy chain having an amino acid sequence having at least 95% identity to an amino acid sequence of SEQ ID NO:403; and (ii) a light chain having an amino acid sequence having at least 95% identity to an amino acid sequence of SEQ ID NO: 404; or (l) (i) a heavy chain having an amino acid sequence having at least 95% identity to an amino acid sequence of SEQ ID NO:437; and (ii) a light chain having an amino acid sequence having at least 95% identity to an amino acid sequence of SEQ ID NO:438.

Embodiment C27 provides the method of embodiment C26, wherein the antibody that binds Sdc2 comprises: (a) (i) a heavy chain having an amino acid sequence SEQ ID NO:63; and (ii) a light chain having an amino acid sequence SEQ ID NO:64; (b) (i) a heavy chain having an amino acid sequence SEQ ID NO: 97; and (ii) a light chain having an amino acid sequence SEQ ID NO:98; (c) (i) a heavy chain having an amino acid sequence SEQ ID NO:131; and (ii) a light chain having an amino acid sequence SEQ ID NO:132; (d) (i) a heavy chain having an amino acid sequence SEQ ID NO:165; and (ii) a light chain having an amino acid sequence SEQ ID NO:166; (e) (i) a heavy chain having an amino acid sequence SEQ ID NO:199; and (ii) a light chain having an amino acid sequence SEQ ID NO:200; (f) (i) a heavy chain having an amino acid sequence SEQ ID NO:233; and (ii) a light chain having an amino acid sequence SEQ ID NO:234; (g) (i) a heavy chain having an amino acid sequence SEQ ID NO:267; and (ii) a light chain having an amino acid sequence SEQ ID NO:268; (h) (i) a heavy chain having an amino acid sequence SEQ ID NO:301; and (ii) a light chain having an amino acid sequence SEQ ID NO:302; (i) (i) a heavy chain having an amino acid sequence SEQ ID NO:335; and (ii) a light chain having an amino acid sequence SEQ ID NO:336; (j) (i) a heavy chain having an amino acid sequence SEQ ID NO:369; and (ii) a light chain having an amino acid sequence SEQ ID NO:370; (k) (i) a heavy chain having an amino acid sequence SEQ ID NO:403; and (ii) a light chain having an amino acid sequence SEQ ID NO:404; or (l) (i) a heavy chain having an amino acid sequence SEQ ID NO:437; and (ii) a light chain having an amino acid sequence SEQ ID NO:438.

Embodiment C28 provides the method of any one of embodiments C21 to C25, wherein the antibody is a humanized antibody or a fully human antibody.

Embodiment C29 provides the method of any one of embodiments C21 to C28, wherein the antibody is an IgG antibody.

Embodiment C30 provides the method of embodiment C29, wherein the IgG antibody is an IgG1, IgG2, IgG3, or IgG4 antibody.

Embodiment C31 provides the method of any one of embodiments C21 to C25, C29 or C30, wherein the antibody comprises a kappa light chain.

Embodiment C32 provides the method of any one of embodiments C21 to C25, C29 or C30, wherein the antibody comprises a lambda light chain.

Embodiment C33 provides the method of any one of embodiments C21 to C32, wherein the antibody is a monoclonal antibody.

### 8.1 Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiment or portions thereof.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations. It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

What is claimed is:

1. An antibody that binds Sdc2 comprising:
(a.) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:61; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:62;
(b.) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:95; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:96;
(c.) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:129; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:130;
(d.) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:163; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 164;
(e.) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:197; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO: 198;
(f.) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:231; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:232;
(g.) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:265; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:266;
(h.) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:299; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:300;
(i.) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:333; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:334;
(j.) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:367; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:368;
(k.) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:401; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:402; or
(l.) (i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:435; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:436.

2. The antibody of claim 1, wherein
(i) the VH CDR1, VH CDR2, VH CDR3, VL CDR1, VL CDR2, and VL CDR3 amino acid sequences are according to the Kabat numbering system;
(ii) the VH CDR1, VH CDR2, VH CDR3, VL CDR1, VL CDR2, and VL CDR3 amino acid sequences are according to the Chothia numbering system;
(iii) the VH CDR1, VH CDR2, VH CDR3, VL CDR1, VL CDR2, and VL CDR3 amino acid sequences are according to the AbM numbering system;
(iv) the VH CDR1, VH CDR2, VH CDR3, VL CDR1, VL CDR2, and VL CDR3 amino acid sequences are according to the Contact numbering system; or
(v) the VH CDR1, VH CDR2, VH CDR3, VL CDR1, VL CDR2, and VL CDR3 amino acid sequences are according to the IMGT numbering system.

3. The antibody of claim 1 or 2, comprising:
(I.) (A) (i) a VH comprising a VH CDR1, a VH CDR2, and a VH CDR3 having an amino acid sequence of SEQ ID NOs: 31, 32 and 33, respectively; and (ii) a VL comprising a VL CDR1, a VL CDR2, and a VL CDR3 having an amino acid sequence of SEQ ID NOs: 34, 35 and 36, respectively;
(B) (i) a VH comprising a VH CDR1, a VH CDR2, and a VH CDR3 having an amino acid sequence of SEQ ID NOs: 37, 38 and 39, respectively; and (ii) a VL comprising a VL CDR1, a VL CDR2, and a VL CDR3 having an amino acid sequence of SEQ ID NOs: 40, 41 and 42, respectively;
(C) (i) a VH comprising a VH CDR1, a VH CDR2, and a VH CDR3 having an amino acid sequence of SEQ ID NOs: 43, 44 and 45, respectively; and (ii) a VL comprising a VL CDR1, a VL CDR2, and a VL CDR3 having an amino acid sequence of SEQ ID NOs: 46, 47 and 48, respectively;
(D) (i) a VH comprising a VH CDR1, a VH CDR2, and a VH CDR3 having an amino acid sequence of SEQ ID NOs: 49, 50 and 51, respectively; and (ii) a VL comprising a VL CDR1, a VL CDR2, and a VL CDR3 having an amino acid sequence of SEQ ID NOs: 52, 53 and 54, respectively; or
(E) (i) a VH comprising a VH CDR1, a VH CDR2, and a VH CDR3 having an amino acid sequence of SEQ ID NOs: 55, 56 and 57, respectively; and (ii) a VL comprising a VL CDR1, a VL CDR2, and a VL CDR3 having an amino acid sequence of SEQ ID NOs: 58, 59 and 60, respectively;
(II.) (A) (i) a VH comprising a VH CDR1, a VH CDR2, and a VH CDR3 having an amino acid sequence of SEQ ID NOs: 65, 66 and 67, respectively; and (ii) a VL comprising a VL CDR1, a VL CDR2, and a VL CDR3 having an amino acid sequence of SEQ ID NOs: 68, 89 and 70, respectively;
(B) (i) a VH comprising a VH CDR1, a VH CDR2, and a VH CDR3 having an amino acid sequence of SEQ ID NOs: 71, 72 and 73, respectively; and (ii) a VL comprising a VL CDR1, a VL CDR2, and a VL CDR3 having an amino acid sequence of SEQ ID NOs: 74, 75 and 76, respectively;
(C) (i) a VH comprising a VH CDR1, a VH CDR2, and a VH CDR3 having an amino acid sequence of SEQ ID NOs: 77, 78 and 79, respectively; and (ii) a VL comprising a VL CDR1, a VL CDR2, and a VL CDR3 having an amino acid sequence of SEQ ID NOs: 80, 81 and 82, respectively;
(D) (i) a VH comprising a VH CDR1, a VH CDR2, and a VH CDR3 having an amino acid sequence of SEQ ID NOs: 83, 84 and 85, respectively; and (ii) a VL comprising a VL CDR1, a VL CDR2, and a VL CDR3 having an amino acid sequence of SEQ ID NOs: 86, 87 and 88, respectively; or
(E) (i) a VH comprising a VH CDR1, a VH CDR2, and a VH CDR3 having an amino acid sequence of SEQ ID NOs: 89, 90 and 91, respectively; and (ii) a VL comprising a VL CDR1, a VL CDR2, and a VL CDR3 having an amino acid sequence of SEQ ID NOs: 92, 93 and 94, respectively;
(III.) (A) (i) a VH comprising a VH CDR1, a VH CDR2, and a VH CDR3 having an amino acid sequence of SEQ ID NOs: 99, 100 and 101, respectively; and (ii) a VL comprising a VL CDR1, a VL CDR2, and a VL CDR3 having an amino acid sequence of SEQ ID NOs: 102,103 and 104, respectively;
(B) (i) a VH comprising a VH CDR1, a VH CDR2, and a VH CDR3 having an amino acid sequence of SEQ ID NOs: 105, 106 and 107, respectively; and (ii) a VL comprising a VL CDR1, a VL CDR2, and a VL CDR3 having an amino acid sequence of SEQ ID NOs: 108, 109 and 110, respectively;
(C) (i) a VH comprising a VH CDR1, a VH CDR2, and a VH CDR3 having an amino acid sequence of SEQ ID NOs: 111, 112 and 113, respectively; and (ii) a VL comprising a VL CDR1, a VL CDR2, and a VL CDR3 having an amino acid sequence of SEQ ID NOs: 114, 115 and 116, respectively;
(D) (i) a VH comprising a VH CDR1, a VH CDR2, and a VH CDR3 having an amino acid sequence of SEQ ID NOs: 117, 118 and 119, respectively; and (ii) a sequence of SEQ ID NOs: 120, 121 and 122, respectively; or
(E) (i) a VH comprising a VH CDR1, a VH CDR2, and a VH CDR3 having an amino acid sequence of SEQ ID NOs: 123, 124 and 125, respectively; and (ii) a VL comprising a VL CDR1, a VL CDR2, and a VL CDR3 having an amino acid sequence of SEQ ID NOs: 126, 127 and 128, respectively;
(IV.) (A) (i) a VH comprising a VH CDR1, a VH CDR2, and a VH CDR3 having an amino acid sequence of SEQ ID NOs: 133, 134 and 135, respectively; and (ii) a VL comprising a VL CDR1, a VL CDR2, and a VL CDR3 having an amino acid sequence of SEQ ID NOs: 136, 137 and 138, respectively;
(B) (i) a VH comprising a VH CDR1, a VH CDR2, and a VH CDR3 having an amino acid sequence of SEQ ID NOs: 139, 140 and 141, respectively; and (ii) a VL comprising a VL CDR1, a VL CDR2, and a VL CDR3 having an amino acid sequence of SEQ ID NOs: 142, 143 and 144, respectively;
(C) (i) a VH comprising a VH CDR1, a VH CDR2, and a VH CDR3 having an amino acid sequence of SEQ ID NOs: 145, 146 and 147, respectively; and (ii) a VL comprising a VL CDR1, a VL CDR2, and a VL CDR3 having an amino acid sequence of SEQ ID NOs: 148, 149 and 150, respectively;
(D) (i) a VH comprising a VH CDR1, a VH CDR2, and a VH CDR3 having an amino acid sequence of SEQ ID NOs: 151, 152 and 153, respectively; and (ii) a VL comprising a VL CDR1, a VL CDR2, and a VL CDR3 having an amino acid sequence of SEQ ID NOs: 154, 155 and 156, respectively; or
(E) (i) a VH comprising a VH CDR1, a VH CDR2, and a VH CDR3 having an amino acid sequence of SEQ ID NOs: 157, 158 and 159, respectively; and (ii) a VL comprising a VL CDR1, a VL CDR2, and a VL CDR3 having an amino acid sequence of SEQ ID NOs: 160, 161 and 162, respectively;
(V.) (A) (i) a VH comprising a VH CDR1, a VH CDR2, and a VH CDR3 having an amino acid sequence of SEQ ID NOs: 167, 168 and 169, respectively; and (ii) a sequence of SEQ ID NOs: 170, 171 and 172, respectively;
(B) (i) a VH comprising a VH CDR1, a VH CDR2, and a VH CDR3 having an amino acid sequence of SEQ ID NOs: 173, 174 and 175, respectively; and (ii) a VL comprising a VL CDR1, a VL CDR2, and a VL CDR3 having an amino acid sequence of SEQ ID NOs: 176, 177 and 178, respectively;
(C) (i) a VH comprising a VH CDR1, a VH CDR2, and a VH CDR3 having an amino acid sequence of SEQ ID NOs: 179, 180 and 181, respectively; and (ii) a VL comprising a VL CDR1, a VL CDR2, and a VL CDR3 having an amino acid sequence of SEQ ID NOs: 182, 183 and 184, respectively;
(D) (i) a VH comprising a VH CDR1, a VH CDR2, and a VH CDR3 having an amino acid sequence of SEQ ID NOs: 185, 186 and 187, respectively; and (ii) a VL comprising a VL CDR1, a VL CDR2, and a VL CDR3 having an amino acid sequence of SEQ ID NOs: 188, 189 and 190, respectively; or
(E) (i) a VH comprising a VH CDR1, a VH CDR2, and a VH CDR3 having an amino acid sequence of SEQ ID NOs: 191, 192 and 193, respectively; and (ii) a VL comprising a VL CDR1, a VL CDR2, and a VL CDR3 having an amino acid sequence of SEQ ID NOs: 194, 195 and 196, respectively;
(VI.) (A) (i) a VH comprising a VH CDR1, a VH CDR2, and a VH CDR3 having an amino acid sequence of SEQ ID NOs: 201, 202 and 203, respectively; and (ii) a VL comprising a VL CDR1, a VL CDR2, and a VL CDR3 having an amino acid sequence of SEQ ID NOs: 204, 205 and 206, respectively;
(B) (i) a VH comprising a VH CDR1, a VH CDR2, and a VH CDR3 having an amino acid sequence of SEQ ID NOs: 207, 208 and 209, respectively; and (ii) a VL comprising a VL CDR1, a VL CDR2, and a VL CDR3 having an amino acid sequence of SEQ ID NOs: 210, 211 and 212, respectively;
(C) (i) a VH comprising a VH CDR1, a VH CDR2, and a VH CDR3 having an amino acid sequence of SEQ ID NOs: 213, 214 and 215, respectively; and (ii) a sequence of SEQ ID NOs: 216, 217 and 218, respectively;
(D) (i) a VH comprising a VH CDR1, a VH CDR2, and a VH CDR3 having an amino acid sequence of SEQ ID NOs: 219, 220 and 221, respectively; and (ii) a VL comprising a VL CDR1, a VL CDR2, and a VL CDR3 having an amino acid sequence of SEQ ID NOs: 222, 223 and 224, respectively; or
(E) (i) a VH comprising a VH CDR1, a VH CDR2, and a VH CDR3 having an amino acid sequence of SEQ ID NOs: 225, 226 and 227, respectively; and (ii) a VL comprising a VL CDR1, a VL CDR2, and a VL CDR3 having an amino acid sequence of SEQ ID NOs: 228, 229 and 230, respectively;
(VII.) (A) (i) a VH comprising a VH CDR1, a VH CDR2, and a VH CDR3 having an amino acid sequence of SEQ ID NOs: 235, 236 and 237, respectively; and (ii) a VL comprising a VL CDR1, a VL CDR2, and a VL CDR3 having an amino acid sequence of SEQ ID NOs: 238, 239 and 240, respectively;
(B) (i) a VH comprising a VH CDR1, a VH CDR2, and a VH CDR3 having an amino acid sequence of SEQ ID NOs: 241, 242 and 243, respectively; and (ii) a VL comprising a VL CDR1, a VL CDR2, and a VL CDR3 having an amino acid sequence of SEQ ID NOs: 244, 245 and 246, respectively;
(C) (i) a VH comprising a VH CDR1, a VH CDR2, and a VH CDR3 having an amino acid sequence of SEQ ID NOs: 247, 248 and 249, respectively; and (ii) a VL comprising a VL CDR1, a VL CDR2, and a VL CDR3 having an amino acid sequence of SEQ ID NOs: 250, 251 and 252, respectively;
(D) (i) a VH comprising a VH CDR1, a VH CDR2, and a VH CDR3 having an amino acid sequence of SEQ ID NOs: 253, 254 and 255, respectively; and (ii) a VL comprising a VL CDR1, a VL CDR2, and a VL CDR3 having an amino acid sequence of SEQ ID NOs: 256, 257 and 258, respectively; or
(E) (i) a VH comprising a VH CDR1, a VH CDR2, and a VH CDR3 having an amino acid sequence of SEQ ID NOs: 259, 260 and 261, respectively; and (ii) a sequence of SEQ ID NOs: 262, 263 and 264, respectively;
(VIII.) (A) (i) a VH comprising a VH CDR1, a VH CDR2, and a VH CDR3 having an amino acid sequence of SEQ ID NOs: 269, 270 and 271, respectively; and (ii) a VL comprising a VL CDR1, a VL CDR2, and a VL CDR3 having an amino acid sequence of SEQ ID NOs: 272, 273 and 274, respectively;
(B) (i) a VH comprising a VH CDR1, a VH CDR2, and a VH CDR3 having an amino acid sequence of SEQ ID NOs: 275, 276 and 277, respectively; and (ii) a VL comprising a VL CDR1, a VL CDR2, and a VL CDR3 having an amino acid sequence of SEQ ID NOs: 278, 279 and 280, respectively;
(C) (i) a VH comprising a VH CDR1, a VH CDR2, and a VH CDR3 having an amino acid sequence of SEQ ID NOs: 281, 282 and 283, respectively; and (ii) a VL comprising a VL CDR1, a VL CDR2, and a VL CDR3 having an amino acid sequence of SEQ ID NOs: 284, 285 and 286, respectively;
(D) (i) a VH comprising a VH CDR1, a VH CDR2, and a VH CDR3 having an amino acid sequence of SEQ ID NOs: 287, 288 and 289, respectively; and (ii) a VL comprising a VL CDR1, a VL CDR2, and a VL CDR3 having an amino acid sequence of SEQ ID NOs: 290, 292 and 292, respectively; or
(E) (i) a VH comprising a VH CDR1, a VH CDR2, and a VH CDR3 having an amino acid sequence of SEQ ID NOs: 293, 294 and 295, respectively; and (ii) a VL comprising a VL CDR1, a VL CDR2, and a VL CDR3 having an amino acid sequence of SEQ ID NOs: 296, 297 and 298, respectively;
(IX.) (A) (i) a VH comprising a VH CDR1, a VH CDR2, and a VH CDR3 having an amino acid sequence of SEQ ID NOs: 303, 304 and 305, respectively; and (ii) a VL comprising a VL CDR1, a VL CDR2, and a VL CDR3 having an amino acid sequence of SEQ ID NOs: 306, 307 and 308, respectively;
(B) (i) a VH comprising a VH CDR1, a VH CDR2, and a VH CDR3 having an amino acid sequence of SEQ ID NOs: 309, 310 and 311, respectively; and (ii) a sequence of SEQ ID NOs: 312, 313 and 314, respectively;
(C) (i) a VH comprising a VH CDR1, a VH CDR2, and a VH CDR3 having an amino acid sequence of SEQ ID NOs: 315, 316 and 317, respectively; and (ii) a VL comprising a VL CDR1, a VL CDR2, and a VL CDR3 having an amino acid sequence of SEQ ID NOs: 318, 319 and 320, respectively;
(D) (i) a VH comprising a VH CDR1, a VH CDR2, and a VH CDR3 having an amino acid sequence of SEQ ID NOs: 321, 322 and 323, respectively; and (ii) a VL comprising a VL CDR1, a VL CDR2, and a VL CDR3 having an amino acid sequence of SEQ ID NOs: 324, 325 and 326, respectively; or
(E) (i) a VH comprising a VH CDR1, a VH CDR2, and a VH CDR3 having an amino acid sequence of SEQ ID NOs: 327, 328 and 329, respectively; and (ii) a VL comprising a VL CDR1, a VL CDR2, and a VL CDR3 having an amino acid sequence of SEQ ID NOs: 330, 331 and 332, respectively;
(X.) (A) (i) a VH comprising a VH CDR1, a VH CDR2, and a VH CDR3 having an amino acid sequence of SEQ ID NOs: 337, 338 and 339, respectively; and (ii) a VL comprising a VL CDR1, a VL CDR2, and a VL CDR3 having an amino acid sequence of SEQ ID NOs: 340, 341 and 342, respectively;
(B) (i) a VH comprising a VH CDR1, a VH CDR2, and a VH CDR3 having an amino acid sequence of SEQ ID NOs: 343, 344 and 345, respectively; and (ii) a VL comprising a VL CDR1, a VL CDR2, and a VL CDR3 having an amino acid sequence of SEQ ID NOs: 346, 347 and 348, respectively;
(C) (i) a VH comprising a VH CDR1, a VH CDR2, and a VH CDR3 having an amino acid sequence of SEQ ID NOs: 349, 350 and 351, respectively; and (ii) a VL comprising a VL CDR1, a VL CDR2, and a VL CDR3 having an amino acid sequence of SEQ ID NOs: 352, 353 and 354, respectively;
(D) (i) a VH comprising a VH CDR1, a VH CDR2, and a VH CDR3 having an amino acid sequence of SEQ ID NOs: 355, 356 and 357, respectively; and (ii) a sequence of SEQ ID NOs: 358, 359 and 360, respectively; or
(E) (i) a VH comprising a VH CDR1, a VH CDR2, and a VH CDR3 having an amino acid sequence of SEQ ID NOs: 361, 362 and 363, respectively; and (ii) a VL comprising a VL CDR1, a VL CDR2, and a VL CDR3 having an amino acid sequence of SEQ ID NOs: 364, 365 and 366, respectively;
(XI.) (A) (i) a VH comprising a VH CDR1, a VH CDR2, and a VH CDR3 having an amino acid sequence of SEQ ID NOs: 371, 372 and 373, respectively; and (ii) a VL comprising a VL CDR1, a VL CDR2, and a VL CDR3 having an amino acid sequence of SEQ ID NOs: 374, 375 and 376, respectively;
(B) (i) a VH comprising a VH CDR1, a VH CDR2, and a VH CDR3 having an amino acid sequence of SEQ ID NOs: 377, 378 and 379, respectively; and (ii) a VL comprising a VL CDR1, a VL CDR2, and a VL CDR3 having an amino acid sequence of SEQ ID NOs: 380, 381 and 382, respectively;
(C) (i) a VH comprising a VH CDR1, a VH CDR2, and a VH CDR3 having an amino acid sequence of SEQ ID NOs: 383, 384 and 385, respectively; and (ii) a VL comprising a VL CDR1, a VL CDR2, and a VL CDR3 having an amino acid sequence of SEQ ID NOs: 386, 387 and 388, respectively;
(D) (i) a VH comprising a VH CDR1, a VH CDR2, and a VH CDR3 having an amino acid sequence of SEQ ID NOs: 389, 390 and 391, respectively; and (ii) a VL comprising a VL CDR1, a VL CDR2, and a VL CDR3 having an amino acid sequence of SEQ ID NOs: 392, 393 and 394, respectively; or
(E) (i) a VH comprising a VH CDR1, a VH CDR2, and a VH CDR3 having an amino acid sequence of SEQ ID NOs: 395, 396 and 397, respectively; and (ii) a VL comprising a VL CDR1, a VL CDR2, and a VL CDR3 having an amino acid sequence of SEQ ID NOs: 398, 399 and 400, respectively; or
(XII.) (A) (i) a VH comprising a VH CDR1, a VH CDR2, and a VH CDR3 having an amino acid sequence of SEQ ID NOs: 405, 406 and 407, respectively; and (ii) a sequence of SEQ ID NOs: 408, 409 and 410, respectively;
(B) (i) a VH comprising a VH CDR1, a VH CDR2, and a VH CDR3 having an amino acid sequence of SEQ ID NOs: 411, 412 and 413, respectively; and (ii) a VL comprising a VL CDR1, a VL CDR2, and a VL CDR3 having an amino acid sequence of SEQ ID NOs: 414, 415 and 416, respectively;
(C) (i) a VH comprising a VH CDR1, a VH CDR2, and a VH CDR3 having an amino acid sequence of SEQ ID NOs: 417, 418 and 419, respectively; and (ii) a VL comprising a VL CDR1, a VL CDR2, and a VL CDR3 having an amino acid sequence of SEQ ID NOs: 420, 421 and 422, respectively;
(D) (i) a VH comprising a VH CDR1, a VH CDR2, and a VH CDR3 having an amino acid sequence of SEQ ID NOs: 423, 424 and 425, respectively; and (ii) a VL comprising a VL CDR1, a VL CDR2, and a VL CDR3 having an amino acid sequence of SEQ ID NOs: 426, 427 and 428, respectively; or
(E) (i) a VH comprising a VH CDR1, a VH CDR2, and a VH CDR3 having an amino acid sequence of SEQ ID NOs: 429, 430 and 431, respectively; and (ii) a VL comprising a VL CDR1, a VL CDR2, and a VL CDR3 having an amino acid sequence of SEQ ID NOs: 432, 433 and 434, respectively.

4. The antibody of any one of claims 1 to 3, comprising:
(a.) (i) a VH having an amino acid sequence having at least 95% identity to an amino acid sequence of SEQ ID NO:61; and (ii) a VL having an amino acid sequence having at least 95% identity to an amino acid sequence of SEQ ID NO:62;
(b.) (i) a VH having an amino acid sequence having at least 95% identity to an amino acid sequence of SEQ ID NO:95; and (ii) a VL having an amino acid sequence having at least 95% identity to an amino acid sequence of SEQ ID NO:96;
(c.) (i) a VH having an amino acid sequence having at least 95% identity to an amino acid sequence of SEQ ID NO: 129; and (ii) a VL having an amino acid sequence having at least 95% identity to an amino acid sequence of SEQ ID NO:130;
(d.) (i) a VH having an amino acid sequence having at least 95% identity to an amino acid sequence of SEQ ID NO: 163; and (ii) a VL having an amino acid sequence having at least 95% identity to an amino acid sequence of SEQ ID NO:164;
(e.) (i) a VH having an amino acid sequence having at least 95% identity to an amino acid sequence of SEQ ID NO:197; and (ii) a VL having an amino acid sequence having at least 95% identity to an amino acid sequence of SEQ ID NO:198;
(f.) (i) a VH having an amino acid sequence having at least 95% identity to an amino acid sequence of SEQ ID NO:231; and (ii) a VL having an amino acid sequence having at least 95% identity to an amino acid sequence of SEQ ID NO:232;
(g.) (i) a VH having an amino acid sequence having at least 95% identity to an amino acid sequence of SEQ ID NO:265; and (ii) a VL having an amino acid sequence having at least 95% identity to an amino acid sequence of SEQ ID NO:266;
(h.) (i) a VH having an amino acid sequence having at least 95% identity to an amino acid sequence of SEQ ID NO:299; and (ii) a VL having an amino acid sequence having at least 95% identity to an amino acid sequence of SEQ ID NO:300;
(i.) (i) a VH having an amino acid sequence having at least 95% identity to an amino acid sequence of SEQ ID NO:333; and (ii) a VL having an amino acid sequence having at least 95% identity to an amino acid sequence of SEQ ID NO:334;
(j.) (i) a VH having an amino acid sequence having at least 95% identity to an amino acid sequence of SEQ ID NO:367; and (ii) a VL having an amino acid sequence having at least 95% identity to an amino acid sequence of SEQ ID NO:368;
(k.) (i) a VH having an amino acid sequence having at least 95% identity to an amino acid sequence of SEQ ID NO:401; and (ii) a VL having an amino acid sequence having at least 95% identity to an amino acid sequence of SEQ ID NO:402; or
(l.) (i) a VH having an amino acid sequence having at least 95% identity to an amino acid sequence of SEQ ID NO:435; and (ii) a VL having an amino acid sequence having at least 95% identity to an amino acid sequence of SEQ ID NO:436.

5. The antibody of claim 4, comprising:
(a.) (i) a VH having an amino acid sequence SEQ ID NO:61; and (ii) a VL having an amino acid sequence SEQ ID NO:62;
(b.) (i) a VH having an amino acid sequence SEQ ID NO:95; and (ii) a VL having an amino acid sequence SEQ ID NO:96;
(c.) (i) a VH having an amino acid sequence SEQ ID NO:129; and (ii) a VL having an amino acid sequence SEQ ID NO:130;
(d.) (i) a VH having an amino acid sequence SEQ ID NO: 163; and (ii) a VL having an amino acid sequence SEQ ID NO:164;
(e.) (i) a VH having an amino acid sequence SEQ ID NO:197; and (ii) a VL having an amino acid sequence SEQ ID NO:198;
(f.) (i) a VH having an amino acid sequence SEQ ID NO:231; and (ii) a VL having an amino acid sequence SEQ ID NO:232;
(g.) (i) a VH having an amino acid sequence SEQ ID NO:265; and (ii) a VL having an amino acid sequence SEQ ID NO:266;
(h.) (i) a VH having an amino acid sequence SEQ ID NO:299; and (ii) a VL having an amino acid sequence SEQ ID NO:300;
(i.) (i) a VH having an amino acid sequence SEQ ID NO:333; and (ii) a VL having an amino acid sequence SEQ ID NO:334;
(j.) (i) a VH having an amino acid sequence SEQ ID NO:367; and (ii) a VL having an amino acid sequence SEQ ID NO:368;
(k.) (i) a VH having an amino acid sequence SEQ ID NO:401; and (ii) a VL having an amino acid sequence SEQ ID NO:402; or
(l.) (i) a VH having an amino acid sequence SEQ ID NO:435; and (ii) a VL having an amino acid sequence SEQ ID NO:436.

6. The antibody of claims 1 to 4, comprising:
(a.) (i) a heavy chain having an amino acid sequence having at least 95% identity to an amino acid sequence of SEQ ID NO:63; and (ii) a light chain having an amino acid sequence having at least 95% identity to an amino acid sequence of SEQ ID NO: 64;
(b.) (i) a heavy chain having an amino acid sequence having at least 95% identity to an amino acid sequence of SEQ ID NO:97; and (ii) a light chain having an amino acid sequence having at least 95% identity to an amino acid sequence of SEQ ID NO: 98;
(c.) (i) a heavy chain having an amino acid sequence having at least 95% identity to an amino acid sequence of SEQ ID NO:131; and (ii) a light chain having an amino acid sequence having at least 95% identity to an amino acid sequence of SEQ ID NO:132;
(d.) (i) a heavy chain having an amino acid sequence having at least 95% identity to an amino acid sequence of SEQ ID NO:165; and (ii) a light chain having an amino acid sequence having at least 95% identity to an amino acid sequence of SEQ ID NO:166;
(e.) (i) a heavy chain having an amino acid sequence having at least 95% identity to an amino acid sequence of SEQ ID NO:199; and (ii) a light chain having an amino acid sequence having at least 95% identity to an amino acid sequence of SEQ ID NO:200;
(f.) (i) a heavy chain having an amino acid sequence having at least 95% identity to an amino acid sequence of SEQ ID NO:233; and (ii) a light chain having an amino acid sequence having at least 95% identity to an amino acid sequence of SEQ ID NO:234;
(g.) (i) a heavy chain having an amino acid sequence having at least 95% identity to an amino acid sequence of SEQ ID NO:267; and (ii) a light chain having an amino acid sequence having at least 95% identity to an amino acid sequence of SEQ ID NO:268;
(h.) (i) a heavy chain having an amino acid sequence having at least 95% identity to an amino acid sequence of SEQ ID NO:301; and (ii) a light chain having an amino acid sequence having at least 95% identity to an amino acid sequence of SEQ ID NO:302;
(i.) (i) a heavy chain having an amino acid sequence having at least 95% identity to an amino acid sequence of SEQ ID NO:335; and (ii) a light chain having an amino acid sequence having at least 95% identity to an amino acid sequence of SEQ ID NO:336;
(j.) (i) a heavy chain having an amino acid sequence having at least 95% identity to an amino acid sequence of SEQ ID NO:369; and (ii) a light chain having an amino acid sequence having at least 95% identity to an amino acid sequence of SEQ ID NO:370;
(k.) (i) a heavy chain having an amino acid sequence having at least 95% identity to an amino acid sequence of SEQ ID NO:403; and (ii) a light chain having an amino acid sequence having at least 95% identity to an amino acid sequence of SEQ ID NO:404; or
(l.) (i) a heavy chain having an amino acid sequence having at least 95% identity to an amino acid sequence of SEQ ID NO:437; and (ii) a light chain having an amino acid sequence having at least 95% identity to an amino acid sequence of SEQ ID NO:438.

7. The antibody of claim 6, comprising:
(a.) (i) a heavy chain having an amino acid sequence SEQ ID NO:63; and (ii) a light chain having an amino acid sequence SEQ ID NO:64;
(b.) (i) a heavy chain having an amino acid sequence SEQ ID NO:97; and (ii) a light chain having an amino acid sequence SEQ ID NO:98;
(c.) (i) a heavy chain having an amino acid sequence SEQ ID NO:131; and (ii) a light chain having an amino acid sequence SEQ ID NO:132;
(d.) (i) a heavy chain having an amino acid sequence SEQ ID NO:165; and (ii) a light chain having an amino acid sequence SEQ ID NO:166;
(e.) (i) a heavy chain having an amino acid sequence SEQ ID NO:199; and (ii) a light chain having an amino acid sequence SEQ ID NO:200;
(f.) (i) a heavy chain having an amino acid sequence SEQ ID NO:233; and (ii) a light chain having an amino acid sequence SEQ ID NO:234;
(g.) (i) a heavy chain having an amino acid sequence SEQ ID NO:267; and (ii) a light chain having an amino acid sequence SEQ ID NO:268;
(h.) (i) a heavy chain having an amino acid sequence SEQ ID NO:301; and (ii) a light chain having an amino acid sequence SEQ ID NO:302;
(i.) (i) a heavy chain having an amino acid sequence SEQ ID NO:335; and (ii) a light chain having an amino acid sequence SEQ ID NO:336;
(j.) (i) a heavy chain having an amino acid sequence SEQ ID NO:369; and (ii) a light chain having an amino acid sequence SEQ ID NO:370;
(k.) (i) a heavy chain having an amino acid sequence SEQ ID NO:403; and (ii) a light chain having an amino acid sequence SEQ ID NO:404; or
(l.) (i) a heavy chain having an amino acid sequence SEQ ID NO:437; and (ii) a light chain having an amino acid sequence SEQ ID NO:438.

8. The antibody of claims 1 to 5, wherein the antibody is a humanized antibody or a fully human antibody.

9. The antibody of any one of claims 1 to 8, wherein the antibody is an IgG antibody.

10. The antibody of claim 9, wherein the IgG antibody is an IgG1, IgG2, IgG3, or IgG4 antibody.

11. The antibody of any one of claim 1 to 5, 9 or 10, wherein the antibody comprises a kappa light chain.

12. The antibody of any one of claim 1 to 5, 9 or 10, wherein the antibody comprises a lambda light chain.

13. The antibody of any one of claims 1 to 12, wherein the antibody is a monoclonal antibody.

14. The antibody of any one of claims 1 to 13, wherein the antibody specifically binds to Sdc2.

15. The antibody of any one of claims 1 to 14, wherein the Sdc2 is present on the surface of an endothelial cell.

16. The antibody of any one of claims 1 to 14, wherein the Sdc2 is present on the surface of a neural cell.

17. The antibody of any one of claims 1 to 16, wherein the antibody is multivalent.

18. The antibody of any one of claims 1 to 17, wherein the antibody is a multispecific antibody.

19. A nucleic acid encoding the antibody of any one of claims 1 to 18.

20. A vector comprising the nucleic acid of claim 19.

21. A host cell comprising the vector of claim 20.

22. A kit comprising the vector of claim 20 and packaging for the same.

23. A kit comprising the antibody of any one of claims 1 to 18 and packaging for the same.

24. A pharmaceutical composition comprising the antibody of any one of claims 1 to 18, and a pharmaceutically acceptable carrier.

25. A method of producing the pharmaceutical composition of claim 24, comprising combining the antibody with a pharmaceutically acceptable carrier to obtain the pharmaceutical composition.

26. A method of reducing vascular cell permeability, comprising contacting the vascular cells with the antibody of any one of claims 1 to 18.

27. A method of reducing endothelial cell permeability, comprising contacting the endothelial cells with the antibody of any one of claims 1 to 18.

28. A method of reducing VEGFA-induced endothelial cell permeability, comprising contacting the endothelial cells with the antibody of any one of claims 1 to 18, either before, during or after the endothelial cells are contacted with the VEGFA.

29. A method of reducing vascular permeability in a subject, comprising administering to the subject an effective amount of the antibody of any one of claims 1 to 18.

30. A method of reducing vascular leakage in a subject, comprising administering to the subject an effective amount of the antibody of any one of claims 1 to 18.

31. A method of reducing endothelial permeability in a subject, comprising administering to the subject an effective amount of the antibody of any one of claims 94 to 111.

32. The method of any one of claims 29 to 31, wherein the subject has a disease caused all or in part by cells expressing Sdc2.

33. A method of preventing, treating, or modulating a disease caused all or in part by cells expressing Sdc2, comprising administering to the subject an effective amount of the antibody of any one of claims 1 to 18.

34. The method of claim 33, wherein the cells are endothelial cells.

35. The method of claims 32 to 34, wherein the disease is associated with vascular permeability or vascular leakage.

36. The method of any one of claims 32 to 35, wherein the disease is an acute respiratory distress syndrome (ARDS).

37. The method of claim 36, wherein the ARDS is a COVID-19-induced ARDS.

38. The method of any one of claims 32 to 35, wherein the disease is a neurological disease in which the blood brain barrier (BBB) is altered or disrupted.

39. The method of any one of claims 32 to 35, wherein the disease is Parkinson's Disease.

40. The method of any one of claims 32 to 35, wherein the disease is Alzheimer's disease.

41. The method of any one of claims 32 to 35, wherein the disease is Huntington's Disease.

42. The method of any one of claims 32 to 35, wherein the disease is a peripheral neuropathy.

43. The method of any one of claims 32 to 35, wherein the disease is a traumatic brain injury.

44. The method of any one of claims 32 to 35, wherein the disease is epilepsy.

45. The method of any one of claims 32 to 35, wherein the disease is multiple sclerosis.

46. The method of any one of claims 32 to 35, wherein the disease is a cardiovascular disease.

47. The method of any one of claims 32 to 35, wherein the disease is a myocardial infarction.

48. The method of any one of claims 32 to 35, wherein the disease is congestive heart failure.

49. The method of any one of claims 32 to 35, wherein the disease is a blunt trauma injury.

50. The method of any one of claims 32 to 35, wherein the disease is a peripheral vascular disease.

51. The method of any one of claims 32 to 35, wherein the disease is a lymphedema.

52. The method of any one of claims 32 to 35, wherein the disease is POEMS Syndrome.

53. The method of any one of claims 32 to 35, wherein the disease is a pediatric capillary leak syndrome.

54. The method of any one of claims 32 to 35, wherein the disease is an adult capillary leak syndrome.

55. The method of any one of claims 32 to 35, wherein the disease is a hydrocephalus.

56. The method of any one of claims 32 to 35, wherein the disease is an inflammation-associated edema.

57. The method of any one of claims 32 to 35, wherein the disease is an inflammatory disease.

58. The method of any one of claims 32 to 35, wherein the disease is systemic lupus erythematosus.

59. The method of any one of claims 32 to 35, wherein the disease is rheumatoid arthritis.

60. The method of any one of claims 32 to 35, wherein the disease is a cardiovascular disease.

61. The method of any one of claims 32 to 35, wherein the disease is a neovascular eye disease.

62. The method of any one of claims 32 to 35, wherein the disease is age-related macular degeneration (AMD).

63. The method of any one of claims 32 to 35, wherein the disease is diabetic retinopathy.

64. The method of any one of claims 32 to 35, wherein the disease is a stroke.

65. The method of any one of claims 32 to 35, wherein the disease is an ischemic stroke.

66. The method of any one of claims 32 to 35, wherein the disease is a hemorrhagic stroke.

67. The method of any one of claims 32 to 35, wherein the disease is a cancer.

68. A method of treating a stroke in a subject, comprising administering to the subject an effective amount of the antibody of any one of claims 1 to 18.

69. A method of treating an ischemic stroke in a subject, comprising administering to the subject an effective amount of the antibody of any one of claims 1 to 18.

70. The method of claim 69, wherein the subject has a brain lesion area associated with the ischemic stroke.

71. The method of claim 70, wherein the administration of the antibody results in:
(i) a reduction of endothelial cell permeability in or surrounding the brain lesion area,
wherein optionally the endothelial cell permeability is VEGFA-induced endothelial cell permeability;
(ii) a reduction of vascular permeability in or surrounding the brain lesion area; or
(iii) a reduction in the size of a penumbra, edema or infarct of the brain lesion area, wherein optionally the reduction in size is detected by MRI and/or measured in area or volume.

72. A method of treating a hemorrhagic stroke in a subject, comprising administering to the subject an effective amount of the antibody of any one of claims 1 to 18.

73. A method of reducing eye inflammation is a subject, comprising administering to the subject an effective amount of the antibody of any one of claims 1 to 18.

74. The method of claim 73, wherein the administration of the antibody results in a reduction of one or more inflammation marker in the choroid of an eye of the subject;
wherein optionally the inflammation marker is CD31 or F4/80.

75. The method of claim 74, wherein the administration of the antibody results in substantially the same expression of one or more endothelial marker in the choroid of an eye of the subject,
wherein optionally the endothelial marker is ERG.

76. A method of treating a neovascular eye disease in a subject, comprising administering to the subject an effective amount of the antibody of any one of claims 1 to 18.

77. A method of treating diabetic retinopathy in a subject, comprising administering to the subject an effective amount of the antibody of any one of claims 1 to 18.

78. A method of treating AMD in a subject, comprising administering to the subject an effective amount of the antibody of any one of claims 1 to 18.

79. The method of claim 78, wherein the administration of the antibody results in:
(i) a reduction of the central retinal thickness of an eye of the subject;
wherein optionally the reduction in central retinal thickness is measured by optical coherence tomography (OCT);
(ii) a reduction of endothelial permeability in an eye fundus tissue of the subject,
wherein optionally the endothelial permeability is VEGFA-induced endothelial cell permeability;
wherein optionally the endothelial permeability is measured by fundus fluorescein angiography (FFA);
(iii) a reduction of vascular permeability in an eye fundus tissue of the subject;
wherein optionally the vascular permeability is measured by FFA;
(iv) an upregulation of Dep-1 surface expression on cells in an eye fundus tissue of the subject;
(v) an enhancement of dephosphorylation of vascular endothelial growth factor receptor 2 (VEGFR2) protein at residue Y951 in cells in an eye fundus tissue of the subject;
(vi) a reduction of inflammation in an eye fundus tissue of the subject;
(vii) a reduction in the expression of one or more inflammatory marker in an eye fundus tissue of the subject;
wherein optionally the inflammatory marker is selected from pro-inflammatory cytokines and immune cell surface proteins;
wherein optionally the inflammatory marker is F4/80;
(viii) no change in angiogenesis in an eye fundus tissue of the subject;
wherein optionally the angiogenesis is choroidal neovascularization (CNV); or
(ix) substantially no change in expression of one or more endothelial marker in an eye fundus tissue of the subject;
wherein optionally the endothelial marker is ERG;
wherein optionally wherein the endothelial marker is CD31;
wherein optionally, the eye fundus tissue is selected from retina, macula and choroid of the eye.

80. The method of any one of claims 73 to 79, wherein upon administering of the antibody, the vision acuity of the subject is enhanced.

81. The method of any one of claims 78 to 80, wherein the subject is a human suffering from or at risk of developing AMD.

82. A method of treating a cardiovascular disease in a subject, comprising administering to the subject an effective amount of the antibody of any one of claims 1 to 18.

83. A method of treating congestive heart failure in a subject, comprising administering to the subject an effective amount of the antibody of any one of claims 1 to 18.

84. A method of treating a myocardial infarction in a subject, comprising administering to the subject an effective amount of the antibody of any one of claims 1 to 18.

85. The method of claim 84, wherein the administration of the antibody results in:
(i) a reduction of endothelial permeability in a heart tissue of the subject,
wherein optionally the endothelial permeability is VEGFA-induced endothelial cell permeability;
wherein optionally the endothelial permeability is measured by an Evans Blue assay or a Dextran perfusion assay;
(ii) a reduction of vascular permeability in a heard tissue of the subject;
wherein optionally the vascular permeability is measured by an Evans Blue assay or a Dextran perfusion assay;
(iii) an upregulation of Dep-1 surface expression on cells in a heart tissue of the subject;
(iv) an enhancement of dephosphorylation of vascular endothelial growth factor receptor 2 (VEGFR2) protein at residue Y951 in cells in a heart tissue of the subject;
(v) a reduction of inflammation in a heart tissue of the subject;
(vi) a reduction in the expression of one or more inflammatory marker in a heart tissue of the subject;
wherein optionally the inflammatory marker is selected from pro-inflammatory cytokines and immune cell surface proteins;
wherein optionally the inflammatory marker is CD11b, GM-CSF, MIG, CCL11, IL-3, IL-6, or TNF-α;
wherein optionally reduction of expression of the one or more inflammatory marker occurred within about 24 hours, within about 36 hours, or within about 72 hours after administration of the antibody;
(vii) an enhancement of left ventricular (LV) ejection fraction (LVEF) of the heart of the subject;
(viii) an enhancement of cardiac output of the heart of the subject;
(ix) a reduction of LV end diastolic diameter (LVEDD) of the heart of the subject;
(x) a reduction of LV end systolic diameter (LVESD) of the heart of the subject;
(xi) a reduction of LV end diastolic volume of the heart of the subject;
(xii) a reduction of LV end systolic volume of the heart of the subject;
(xiii) a reduction of LV mass of the heart of the subject;
(xiv) an enhancement of fractional shortening of the heart of the subject;
(xv) an enhancement of ejection fraction of the heart of the subject;
(xvi) a reduction of risk or duration of post-infarct ventricular tachycardia (VT) of the subject;
wherein optionally the post-infarct VT of the subject is measured by an electrocardiogram with programmed stimulation of the heart of the subject;
wherein optionally an increase in the number of stimuli for inducing the VT indicates a reduced risk of post-infarct VT in the subject;
wherein optionally a reduced duration of induced VT under a hypokalemic condition indicates a reduced risk of post-infarct VT in the subject; optionally
wherein a longer cycle length of induced VT indicates a reduced risk of post-infarct VT in the subject; or
(xvii) a reduction of risk for the subject to have a heart failure;
wherein optionally the risk is having the heart failure within 1-3 months following the myocardial infarction.

86. The method of claim 84 or 85, wherein the subject is a human suffering from or at risk of developing a myocardial infarction.

87. The method of any one of claims 29 to 86, wherein the subject is a human.

88. The method of claim 85, wherein the subject is a human subject in need thereof.
